(19) United States
(12) Patent Application Publication
(10) Pub. No.: US 2004/0107461 A1
(43) Pub. Date: Jun. 3, 2004

(54) Glucan chain length domains

(76) Inventors: Padma Commuri, Ankeny, IA (US); Peter L. Keeling, Ames, IA (US); Nona Ramirez, Ames, IA (US); Angela McKean, Ames, IA (US); Zhong Gao, Ames, IA (US); Hanping Guan, Ames, IA (US)

Correspondence Address: NIXON & VENDERHYE P.C., 8th Floor, 1100 North Glebe Road, Arlington, VA, 22201-4714, US

(21) Appl. No.: 10/109,048

(22) Filed: Mar. 29, 2002

Related U.S. Application Data
(60) Provisional application No. 60/279,720, filed on Mar. 30, 2001.

(51) Int. Cl. A01H 1/00; C12N 15/82; C12P 21/06; C07H 21/04; C12P 19/04; C08B 31/00; C12N 5/04

(52) U.S. Cl. 800/284; 435/69.1; 435/193; 435/101; 435/320.1; 435/419; 536/23.2; 536/102

(57) ABSTRACT

The invention relates to a method for changing the glucan chain lengths using fusion protein domains of various starch synthase enzymes in any starch or starch granule producing organism. The invention relates to identification of a GLucan ASSociation domain (herein after referred to as “GLASS” domain) of granule bound starch synthase (GBSS) used in combination with any other GLYcosyl TRransferase domain otherwise referred to as pfam00534-catalytic domain (herein after referred to as “GLYTR” domain) of one or more of any of the other starch synthase enzymes. The invention relates to identifying and using the new and surprising discovery that starch synthases are composed of at least two distinct functional domains herein after labeled as “GLASS” and “GLYTR”. More specifically, this invention relates to the genetic constructs that encode the fusions of the above domains and to the plants transformed with said constructs. The method of invention can thus be used in particular to provide a modified profile of starch granule associated starch synthase (SS) enzymes and by which modified glucan chain lengths of amylopectin and hence, modified starches and or complexes will be generated. This can be done in any organism and more particularly any plant that stores or synthesizes starch in any of its parts, such as potato, sweet potato, cassaya, pea, taro, banana, yam and cereal crops such as rice, maize, wheat, barley, oats, and sorghum.

This application claims priority to provisional patent application serial No. 60/279,720 filed Mar. 30, 2001, the entire contents of which is incorporated herein by reference.

The development of genetic engineering techniques has made it possible to transfer genes from various organisms and plants into other organisms or plants. Although starch has been altered by transformation and mutagenesis in the past, there is still a great need for further starch M modifications in order to meet the ever-increasing industrial need for variations of this natural polymer. Toward this end, there is a need to manipulate the chain lengths of glucan chains in amylopectin in order to provide novel starches with multitude of industrial uses. In order to achieve this goal the present invention is directed at introducing changes in the ratios, composition, and functionality of various enzymes in the starch synthesis pathway.

The present invention relates to novel plants expressing transgenic genes and having an altered ability to produce specialty starch traits with modified glucan chain lengths. Modification of starch biosynthesis pathways by changing the functionality of starch synthase enzymes has an enormous potential for production of new and improved starches. Maize starch is used to produce a wide range of food products (for human and animal consumption) and several industrial products. Several crop varieties are known which produce different types of starch. The type or quality of starch makes it suitable for certain purposes, including particular methods of processing or particular end-uses. For example, U.S. Pat. Nos. 4,789,557, 4,790,997, 4,774,328, 4,770,710, 4,798,735, 4,767,849, 4,801,470, 4,789,738, 4,792,458 and 5,009,911 describe naturally occurring maize mutants producing starches of differing fine structure suitable for use in various food products. Although known mutants produce altered starch, some of these lines are not suitable for crop breeding and/or for the farmers' purposes. By combining various mutants together, such as double, triple and quadruple mutants it is possible to create a variety of plant starches. One key element that is lacking in these starches is the ability to control glucan chain length in the amylopectin molecule. Moreover, such mutant plants often give relatively poor yields and also low gemination rates.

Glucan chain length and chain length distribution are the two key components that determine the functionality of any given starch. Glucan chain lengths can be modified by genetic manipulation of enzymes known to possess other favorable characteristics. For example, by manipulating the function and expression levels of one or more starch synthesizing enzyme genes in a plant, it is possible to significantly alter the type of starch produced. The present invention has led to a new undertstanding of how different starch synthase (SS) enzymes recognize and have specific catalytic capabilities to various lengths of glucan chains (See also, Commuri and Keeling, 2001, The Plant Journal, 25(5), 475-486; and Commuri et al., 2002, in review, The Plant Journal). Comparative analysis have recently been completed on the functionality and catalytic properties of various maize starch synthase enzymes, namely, SSI, SSIIa, SSIIb, granule bound starch synthase (GBSS), and Du1 (SSIII) using glucan substrates with varying average chain lengths (FIGS. 1, 2 &3). Each one of these enzymes possessed different glucan chain length specificities. In maize endosperm, not all the SS enzymes are expressed at the same levels nor localized uniformly in the amyloplast stroma, and/or starch granules. Thus, each form of SS enzyme must contribute in a unique and a specific way in setting the starch structure, and there exists an enormous potential to bring a modification to the structure of starch by alteration of their location of expression and manipulating the levels of activity of these enzymes in the endosperm. Of the five different SS enzymes known to date in maize, GBSS enzyme has the highest affinity to amylopectin followed by SSI (Table 1). It is because of this affinity for its glucan substrate that most of the protein entrapped in the starch granules is comprised of GBSS (˜60%) and SSI enzymes (FIG. 4). Enzymes like SSIIa or SSIIb are undetectable in the granule and are present in low amounts in the amyloplast stroma. The Du1 protein is barely detectable in the granules and is found in reasonable amounts in the amyloplast stroma. Different forms of starch synthases are broadly conserved in evolution and it is reasonable to propose that specific functions have been selected for each one of these isoforms (Myers et al., 2000). For example, in the dull1 (Wang et al., 1993) and sugary 2 (Takeda and Preiss, 1993) mutants that affect enzymes other than SSI (Gao et al., 1998, Harn et al., 1998), both mutations decrease the proportion of the intermediate and average length of long chains (B2 and B3) in the B amylopectin relative to the short (A and B1) chains. This suggests that the enzymes other than SSI are relatively less needed for synthesis of short chains, but are needed for the synthesis of intermediate and longer chains. Furthermore, dull 1 mutation in maize eliminates SS III and the amylopectin from this mutant endosperm is enriched in short chains. In maize, potato and pea, genes for all three forms of SS exist, however, the dominant activity in maize endosperm is SSI, whereas in pea embryos it is SSII, and in potato tubers it is SSIII. It is most interesting to discover in applicants' recent studies using maize that the different SS enzymes have different catalytic capabilities to synthesize various glucan chain lengths. For example, SSI synthesizes shorter chains whereas

TABLE 1
A Comparison of K_{d} values of Maize Granule
Bound Starch Synthase (GBSS) and SS1-2

| | Temperature (° C.) 4 | | Room Temperature (˜23.5) | |
|---|---|---|---|---|
| Type of | GBSS | SSI-2 | GBSS | SSI-2 |
| Glucan | mM | | | |
| Amylose | 0.14 ± 0.01 | 0.35 ± | 0.150 ± 0.01 | 1.06 ± 0.12 |
| | | 0.07^{a} | | |
| Amylo- | 0.015 ± 0.001 | 0.06 ± 0.00 | 0.054 ± 0.004 | 0.07 ± 0.03 |
| pectin | | | | |
| Glycogen | 1.50 ± 0.369 | 1.20 ± 0.03 | (no binding) | 3.38 ± 0.83 |

^{a}= The molar concentration is based on the average outer chain length (OCL) of the substrate molecule (For amylose, amylopectin, and glycogen the apparent average chain lengths are 8-9, 11-12, and 6-7, respectively).

TABLE II
Comparison of K-values of maize SSI and SSI-2,
with α-amylase (porcine pancreas) and
glucoamylase (Aspergillus niger)

| | Maize SSI | | Maize SSI-2 | | α-amylase | | glucoamylase | |
|---|---|---|---|---|---|---|---|---|
| Substrate | mg/mL | mM | mg/mL | mM | mg/mL | mM | mg/mL | mM |
| Starch | 0.261 | 0.069^{a} | 0.241 | 0.065^{a} | 0.539 | 0.144^{a} | 0.090 | 0.024^{a} |
| Amylopectin | 0.217 | 0.076 | 0.242 | 0.078 | 0.602 | 0.212 | 0.030 | 0.010 |

^{a}= Molar concentration is calculated based on the average chain length (C.L.) of the substrate molecule.

GBSS synthesizes very long chains. SSIIa and SSIIb synthesize shorter and more intermediate chains, and SSIII (DuI) synthesizes relatively long chains. Also, these enzymes differ in their glucan binding affinities. SSIIa does not bind to any given glucan of any particular chain lengths that we tested, where as SSIIb displayed partial or minor glucan affinity and SSI binds with greatest affinity to longer glucan chains in amylopectin. This observation explains why SSIIa or SSIIb enzymes are not entrapped in the starch granules and SSI does entrap during the course of starch synthesis. The present invention provides modified starch, and methods of making and using the same, by, for example, structural modification by genetic manipulation of SS enzymes, which is possible due to the presently disclosed discovery of the specificity displayed by the enzymes described herein to a given glucan chain length. This can be accomplished by several ways and listed below and described herein are a few examples: (a) regulating the expression levels of SS enzymes, (b) alteration of the starch biosynthetic pathway by incorporation of the genes encoding one or more enzymes involved in the starch or glycogen biosynthetic pathway, (c) increasing the association of SSIIa, SSIIb and Du1 with the starch granules especially, by engineering entrapment of their corresponding enzymes with the starch granules, and (d) entrapment of fusion proteins of SS enzymes, for example, catalytic domains of SSIIa, SSIIb and Du1 in association with glucan binding domains of GBSS or SSI in the starch granules to bring a change in the glucan chain lengths and distribution and thereby synthesize modified starch.

U.S. Pat. Nos. 5,824,790, 6,130,367, and 5,300,145 describe methods of (a) and (b) of the above. The international application WO 92/11376 describes a method for suppressing amylose formation in potato by transforming potato plant with a construct comprising antisense fragments designed to inhibit the expression of GBSS gene. The Canadian patent application 2,061,143 describes a similar technique for producing amylose free potato starch. The production of modified starches by plants transformed with genes encoding enzymes involved in starch synthesis is described for example in DE-A-19534759, WO 92/14827 in which branching enzyme derived from potato cDNA is used and WO 92/11376 describes an alternative method for antisense suppression of GBSS activity in plants.

However, none of these above mentioned patents describe the combination of different domains of SS enzymes, for example starch association domain (“GLASS” domain) of one enzyme, like GBSS to the catalytic domain (“GLYTR” domain) of another, for example SSIIa, in order to bring a modification to the structure of starch. Surprisingly, the applicants discovery of threading SS enzymes using 3D-PSSM (three-dimensional position-specific scoring matrix) program (Kelley et al., 2000, J. Mol. Boil. 299:499-520) to predict three dimensional structure of SS enzymes and the sequence comparisons revealed two distinct domains for each one of these enzymes (herein after referred to as “GLASS” and “GLYTR”). 3D-PSSM uses structural alignments of homologous proteins of similar three-dimensional structure in the structural classification of proteins (SCOP) database to obtain a structural equivalence of residues. These equivalences are used to extend multiply aligned sequences obtained by standard sequence searches. The resulting large-superfamily based multiple alignment is converted into a PSSM (position specific scoring matrix). Combined with secondary structure matching and solvation potentials, 3D-PSSM can recognize structural and functional relationships beyond state-of the-art sequence methods (Kelly et al., 2000, J. Mol. Biol. 299:499-520). Analysis through 3D-PSSM revealed a conserved two domain 3D structure for all maize starch synthases tested (FIG. 5). This is the first ever report in the scientific literature to model the 3D structure of any starch synthases. Using ProDom database of protein domain families available at the world widee web address Toulouse.inra.fr/prodom.html, Denyer et al., 2001; J. of Plant. Physiol. 158: 479-487, showed identity to three different domains in maize SS enzymes. However, they have not identified a function to Domain I and II and reported that Domain II is also found in Yeast α-amlyase. They reported Domain III as a putative glucosyl transferase domain, but neither provided detailed information as to which group in this family that maize SS enzymes fall under, nor sequence homology or models for 3D-structure of SS protein. Present invention provides the discovery of “GLYTR” domains for all these enzymes as a catalytic domain with significant alignments (using RPS-BLAST 2.2.2; oasis_sapv1.54 database and Domain architectural retrieval tool [DART]) to the glycosyl transferase group-1 domain otherwise referred to as the pfam00534 family (FIG. 6 & Table III). Members of this family are spread across about at least 20+ groups with different mechanism of glycosyl transferase function. Members of the pfam 00534 (PI00534) family transfer UDP, ADP, GDP or CMP linked sugars to a variety of substates including glycogen. The sequence in the catalytic or “GLYTR” domains is highly conserved in starch synthases (Table IV). Furthermore, the present invention relates to the identification of “GLucan ASSociation Domain (“GLASS” Domain), peptides and nucleic acids encoding the same. Glucan chain length specificity is conserved in “GLASS” domain of each form of starch synthase and glycosyl transferase function is conserved in “GLYTR” domain. In addition, starch entrapment function is also embedded in the “GLASS” domain of GBSS and SSI. Also, via genetic means, the present invention provides for generation of starch synthase(s) with novel functionalities by combining various domains from different synthases, i.e. by mixing and matiching functional “GLYTR” and “GLASS” domains from different organisms. Thus, the present invention in particular relates to modification to starch structure by increasing the association of SSIIa, SSIIb and Du1 with the starch granules especially, by engineering entrapment of their corresponding enzymes with the starch granules, and expression and entrapment of fusion proteins of SS enzymes, for example, catalytic domains of SSIIa, SSIIb and Du1 in association with glucan binding domains of GBSS or SSI in the starch granules to bring a change in the glucan chain lengths and distribution and thereby synthesize modified starch. The present invention provides modified plants which contain altered or modified starch synthase domains or polypeptide fusions expressed inside the amyloplast stroma and become associated with the starch granules of economically important crops like maize, potato, rice, oat, wheat, barley, sweet potato, cassaya, taro, sago, yam, banana, pea, etc. These SS enzyme fusions thus expressed will alter or influence the starch structure leading to plants with improved starch properties and modified starches with various industrial uses. Further applications and embodiments of this invention will be explained in detail herein below.

TARI F III

| | | | | | | % |
|---|---|---|---|---|---|---|
| | | | | | | align- |
| ENZYME | Seq. ID No. | Alignment with Pfam 00534 (Glycosyl transferase, Group 1 domain) | | | | ment |
| GBSS | SEQ ID NO:11 | query: | 378 | NKEALQAEVGLPVDRNIPLVAFIGRLEEQKGPDVMAAAIPQLME-MVEDVQIVLLGTGKK | 436 | 91.3 |
| | (query) | Sbjct: | 1 | DREEIRKKLGIKEEKKI--ILFVGRLLPEKGIDLLIEAFKKLKKQLNPNLKLVIVGDGG | 58 | |
| | SEQ ID NO:12 | Query: | 437 | KFERMLMSAEEKFPGKVRAVVKFNAALAHHIMAGADVLAVTSRFEPCGLIQLQGMRYGTP | 496 | |
| | (snjct) | Sbjct: | 59 | EDELKLLALKLGLEDNVIFLGFVPDEDLPELYKSADVFVLPSRYEGFGIVLLEAMACGLP | 118 | |
| | | Query: | 497 | CACASTGGLVDTIIEGKTGFHMGRLSVDCNVVEPADVKKVATTLQRAIK 545 | | |
| | | Sbjct: | 119 | VIATDVGGIPEIVKDGETGL----------LVEPGDVEALAEAIEKLLK 157 | | |
| SSI | SEQ ID NO:13 | Query: | 441 | LPIRPDVPLIGFIGRLDYQKGID-LIQLI--IPDLMREDVQFVMLGSGDPELEDWMRSTE | 497 | 75 |
| | (query) | Sbjct: | 9 | LGIKEEKKIILFVGRLLPEKGIDLLIEAFKKLKKQLNPNLKLVIVGDGEEEDELKLLALK | 68 | |
| | SEQ ID NO:14- | Query: | 498 | SIFKDKFRGWVGF-SVPVSHRITAGCDILLMPSRFEPCGLNQLYAMQYGTVPVVHATGGL | 556 | |
| | (snjct) | Sbjct: | 69 | LGLEDNVI-FLGFVPDEDLPELYKSADVFVLPSRYEGFGIVLLEAMACGLPVIATDVGGI | 127 | |
| | | Query: | 557 | RDTVENFNPFGENG 570 | | |
| | | Sbjct: | 128 | PEIVKD----GETG 137 | | |
| SSIIa | SEQ ID NO:15 | Query: | 540 | LEVRDDVPLLGFIGRLDGQKGVDIIGDAMPWIA---GQDVQLVMLGTGRADLERMLQHLE | 596 | 84.3 |
| | (query) | Sbjct: | 9 | LGIKEEKKIILFVGRLLPEKGIDLLIEAFKKLKKQLNPNLKLVIVGDGEEEDELKLLALK | 68 | |
| | SEQ ID NO:16 | Query: | 597 | REHPNKVRGWVGFSVPMAHRIT--AGADVLVMPSRFEPCGLNQLYAMAYGTVPVVHAVGG | 654 | |
| | (snjct) | Sbjct: | 69 | LGLEDNVI-FLGF-VPDEDLPELYKSADVFVLPSRYEGFGIVLLEAMACGLPVIATDVGG | 126 | |
| | | Query: | 655 | LRDTVAPFDPFGDAGLGWTFDRAEANKLIEALR 687 | | |
| | | Sbjct: | 127 | IPEIVKDGET------GLLVEPGDVEALAEAIE 153 | | |
| SSIIb | SEQ ID NO:17 | Query: | 506 | LQVRDDVPLIGFIGRLDHQKGVDIIADAIHWIA---GQDVQLVMLGTGRADLEDMLRRFE | 562 | 87.2 |
| | (query) | Sbjct: | 9 | LGIKEEKKIILFVGRLLPEKGIDLLIEAFKKLKKQLNPNLKLVIVGDGEEEDELKLLALK | 68 | |
| | SEQ ID NO:18 | Query: | 563 | SEHSDKVRAWVGFS----VPLAHRITAGADILLMPSRFEPCGLNQLYAMAYGTVPVVHAV | 618 | |
| | (snjct) | Sbjct: | 69 | LGLEDNVI-FLGFVPDEDLPELYKS---ADVFVLPSRYEGFGIVLLEAMACGLPVIATDV | 124 | |
| | | Query: | 619 | GGLRDTVAP------FDPFNDTGLGWTFDRAEAN 646 | | |
| | | Sbjct: | 125 | GGIPEIVKDGETGLLVEPGDVEALAEAIEKLLKD 158 | | |
| DuI | SEQ ID NO:19 | Query: | 1478 | PVVGIVTRLTAQKGIHLIKHAIHRTLERNGQVVLLGSAPDSRIQADFVNLANTLHGVNHG | 1537 | 70.3 |
| | (query) | Sbjct: | 16 | KIILFVGRLLPEKGIDLLIEAFKKLKKQLNPNLKLVIVGDGEEEDELKLLALKLGLEDNV | 75 | |
| | SEQ ID NO:20 | Query: | 1538 | QVRLSLTYDEPLSHLIYAGSDFILVPSIFEPCGLTQLVAMRYGTIPIVRKTGGLFDTVFD | 1597 | |
| | (snjct) | Sbjct: | 76 | IFLFGVPDEDLPEL--YKSADVFVLPSRYEGFGIVLLEAMACGLPVIATDVGGIPEIVKD | 133 | |
| | | Query: | 1598 | VDN 1600 | | |
| | | Sbjct: | 134 | GET 136 | | |

TABLE IVa
PIR Multiple Alignment for “GLYTR” Domain of Maize SS enzymes

| Enzyme | | |
|---|---|---|
| (res. # | | |
| of the | SEQ ID | |
| start) | NO:. | CLUSTAL W (1.8) multiple sequence alignment |
| SSIIa-540 | 21 | -----------EVRDDVPLLGFIGRLDGQKGVDIIGDAMPWIAG--QDVQLVMLGTG----- |
| | | RADLERMLQHLEREHPNKVRGWVGFSVPMAHRITAGADVLVMPSRFEPCGLNQLYAMAYGTVPVVHAVGGLRDTVAPFDP- |
| | | FGDAGLGWTFDRAEANKLIEALR--- |
| SSIIb-506 | 22 | ----------LQVRDDVPLIGFIGRLDHQKGVDIIADAIHWIAG--QDVQLVMLGTG----- |
| | | RADLEDMLRRFESEHSDKVRAWVGFSVPLAHRITAGADILLMPSRFEPCGLNQLYAMAYGTVPVVHAVGGLRDTVAPFDP- |
| | | FNDTGLGWTFDRAEAN---------- |
| SSI-441 | 23 | -------- LPIRPDVPLIGFIGRLDYQKGIDLIQLIIPDLMR--EDVQFVMLGSG----- |
| | | DPELEDWMRSTESIFKDKFRGWVGFSVPVSHRITAGCDILLMPSRFEPCGLNQLYAMQYGTVPVVHATGGLRDTVENFNP- |
| | | FGENG--------------------- |
| GBSS-378 | 24 | NKEALQAEVGLPVDRNIPLVAFIGRLEEQKGPDVMAAAIPQLMEMVEDVQIVLLGTG----- |
| | | KKKFERMLMSAEEKFPGKVRAVVKFNAALAHHIMAGADVLAVTSRFEPCGLIQLQGMRYGTPCACASTGGLVDTIIEGKTGFHMGR |
| | | LSVDCNVVEPADVKKVATTLQ |
| Du1-1478 | 25 | -----------------PVVGIVTRLTAQKGIHLIKHAIHRTLE--RNGQVVLLGSAPDS |
| | | RIQADFVNLANTLHGVNHGQVRLSLTYDEPLSHLIYAGSDFILVPSIFEPCGLTQLVAMR |
| | | YGTIPIVRKTGGLFDTVFDVDN-------------------------- |
| | | *::.:: ** *** .:: : .:*.*:**:. .: . .:.* : :. |
| | | .::* * **.*.: :.* ****** ** .* *** .*** **: . |

Footnote:
* = identical amino acid
:. = similar amino acids
- = residues left out

TABLE IVb
PIR Multiple Alignment of SSI and SSII enzymes

| ENZYME | CLUSTAL W (1.8) multiple sequence alignment | SEQ ID NO: |
|---|---|---|
| SSIIA. | MSSAAVSSSSSTFFLALASASPGGRRRARVGSSP---FHTGASLSFAFWAPPSPPRAPRD | 26 |
| SSIIB. | -MPGAISSSSSAFLLPVASSSPR-RRRGSVGAALRSYGYSGAELR-LHWARRGPPQD--G | 27 |
| SSI. | --MATPSAVGAACLLLARAAWP-----AAVG-----------DR-----ARPRRLQR--- | 29 |
| | .: *: .:: :* :: * , ** . * : | |
| SSIIA. | AALVRAEAEAGGKDAPPERSGDAARLPRARRNAVSKRRDPLQPVGRYGSATGNTARTGAA | 26 (cont.d) |
| SSIIB. | AASVRAAAAPAGGESEEAAKSSSSSQAGAVQGSTAKAVDSASPPNPLTSAP---KQSQSA | 27 |
| SSI. | -VLRRRCVAELSREGPAPRPLPPALLA------P-----PLVP----------------- | 29 |
| | . * . . :. .: . . * | |
| SSIIA. | SCQNAALADVEIKSIVAAPPTSIVKFPAPGYRMILPSGDIAPETVLPAPKPLHESPAVDG | 26 (cont.d) |
| SSIIB. | AMQNG------------------------------TSGGSSASTAAPVSGPKADHPSAPV | 27 |
| SSI. | -------------------------------------G-FLAPPAEPTGEPASTPPPVP- | 29 |
| | * . .. *. * *.. | |
| SSIIA. | DSNGIAPPTVEPLVQEATWDFKKYIGFDEPDEAKDDSRVGADDAGSFEHYG-DNDSGPLA | 26 (cont.d) |
| SSIIB. | TKREIDASAVKPEPAGDDARPVESIGIAEPVDAKADAAPATDAAASAPYDREDNEPGPLA | 27 |
| SSI. | -DAGLGDLGLEPE------------GIAEG--SIDNTVVVASEQDSEIVVGKEQARAKVT | 29 |
| | . : ::* *: * : :: :. * :: . :: | |
| SSIIA. | GENVMNVIVVAAECSPWCKTGGLGDVVGALPKALARRGHRVMVVVPRYG------DYVEA | 26 (cont.d) |
| SSIIB. | GPNVMNVVVVASECAPFCKTGGLGDVVGALPKALARRGHRVMVVIPRYG------EYAEA | 27 |
| SSI. | ----QSIVFVTGEASPYAKSGGLGDVCGSLPVALAARGHRVMVVMPRYLNGTSDKNYANA | 29 |
| | .::.*:.*.:*:.*:****** *:** *** ********:*** :*.:* | |
| SSIIA. | FDMGIRKYYKAAGQDLEVNYFHAFIDGVDFVFIDAPLFRHRQDDIYG---GSRQEIMKRM | |
| SSIIB. | RDLGVRRRYKVAGQDSEVTYFHSYIDGVDFVFVEAPPFRHRHNNIYG---GERLDILKRM | |
| SSI. | FYTEKHIRIPCFGGEHEVTFFHEYRDSVDWVFVDHPSY-HRPGNLYGDKFGAFGDNQFRY | |
| | : * : **.:** : *.**:**:: * : ** .::** * : * | |
| SSIIA. | ILFCKVAVEVPWHVPCGGVCYGDGNLVFIANDWHTALLPVYLKAYYRDHGLMQYTRSVLV | 26 (cont.d) |
| SSIIB. | ILFCKAAVEVPWYAPCGGTVYGDGNLVFIANDWHTALLPVYLKAYYRDNGLMQYARSVLV | 27 |
| SSI. | TLLCYAACEAPLILELGGYIYG-QNCMFVVNDWHASLVPVLLAAKYRPYGVYKDSRSILV | 29 |
| | *:* .* *.* ** ** * :*:.****::*:** * * ** *: : :**:** | |
| SSIIA. | IHNIAHQGRGPVDEFPYMDLP-------EHYLQHFELYDPVG-GEHANIFAAGLKMADRV | 26 (cont.d) |
| SSIIB. | IHNIABQGRGPVDDFVNFDLP-------EHYIDHFKLYDNIG-GDHSNVFAAGLKTADRV | 27 |
| SSI. | IHNLAHQGVEPASTYPDLGLPPEWYGALEWVFPEWARRHALDKGEAVNFLKGAVVTADRI | 29 |
| | ***:**** *.. : :.** * : .: . :. *: *.: ..: ***: | |
| SSIIA. | LETLDAGKRQCKAALQRELGLEVRDDVPLLGFIGRLDGQKGVDIIGDAMPWIAGQDVQLV | 26 (cont.d) |
| SSIIB. | FETLDTGKRQCKAALQRQLGLQVRDDVPLIGFIGRLDHQKGVDIIADAIHWIAGQDVQLV | 27 |
| SSI. | VDDLS-GKAKCKGALQKELGLPIRPDVPLIGFIGRLDYQKGIDLIQLIIPDLMREDVQFV | 29 |
| | : *. ** :**.***::*** .* ****:******* ***:*:* : : :***:* | |
| | MLGTGRADLERMLQHLEREHPNKVRGWVGFSVPMAHRITAGADVLVMPSRFEPCGLNQLY | 26 (cont.d) |
| SSIIA. | MLGTGRADLEDMLRRFESEHSDKVRAWVGFSVPLAHRITAGADILLMPSRFEPCGLNQLY | 27 |
| SSIIB. | MLGSGDPELEDWMRSTESIFKDKFRGWVGFSVPVSHRITAGCDILLMPSRFEPCGLNQLY | 29 |
| SSI. | ***:* .:** :: * . *.*. | |
| | *******::*******.*::************** | |
| SSIIA. | AMAYGTVPVVHAVGGLRDTVAPFDPFGDAG--- | 26 (cont.d) |
| SSIIB. | LGWTFDRAEANKLIEALRHCLDTYRKY | 27 |
| SSI. | AMAYGTVPVVHAVGGLRDTVAPFDPFNDTG--- | 29 |
| | LGWTFDRAEANRMIDALSHCLTTYRNY | |
| | AMQYGTVPVVHATGGLRDTVENFNPFGENGEQGTGWAFAPLTTENMFVDIANCN----- | |
| | - | |
| | ** *********.******* *:**.: * **:* ::.:: : :* | |
| SSIIA. | GESWKSLQARGMSQDLSWDHAAELYEDVLVKAKYQW | 26 (cont.d) |
| SSIIB. | KESWRACRARGMAEDLSWDHAAVLYEDVLVKAKYQW | 27 |
| SSI. | ------IYIQGTQVLLGRANEARHVKRLHVGPCR-- | 29 |

TABLE V
Possible, but not limited to Amino acid Sequences for Some of the
Proposed Fusion Proteins

| | |
|---|---|
| A GBSS(61-300) + Du1 (1201-1674) | |
| RRGGRFPSLV VCASAGMNVV FVGAEMAPWS KTGGLGDVLG GLPPAMAANG HRVMVVSPRY DQYKDAWDTS | SEQ ID NO:30 |
| VVSEIKMGDG YETVRFFHCY KRGVDRVFVD HPLFLERVWG KTEEKIYGPV AGTDYRDNQL RFSLLCQAAL | |
| EAPRILSLNN NPYFSGPYGE DVVFVCNDWH TGPLSCYLKS NYQSHGIYRD AKTAFCIHNI SYQGRFAFSD | |
| YPELNLPERF KSSFDFIDGY EKPVEGRKIN WMKAGILEAD RVLTVSPYYA EELISGIARG CELDNIMRLT | |
| GITGIVNGMD VSEWDPSRDK GGIYDNRNGL DYHIPVFGSI AKEPPMHIVH IAVEMAPIAK VGGLGDVVTS | |
| LSRAVQDLGH NVEVILPKYG CLNLSNVKNL QIHQSFSWGG SEINVWRGLV EGLCVYFLEP QNGMFGVGYV | |
| YGRDDDRRFG FFCRSALEFL LQSGSSPNII HCHDWSSAPV AWLHKENYAK SSLANARVVF TIHNLEFGAH | |
| HIGKAMRYCD KATTVSNTYS KEVSGHGAIV PHLGKFYGIL NGIDPDIWDP YNDNFIPVHY TCENVVEGKR | |
| AAKRALQQKF GLQQIDVPVV GIVTRLTAQK GIHLIKHAIH RTLERNGQVV LLGSAPDSRI QADFVNLANT | |
| LHGVNHGQVR LSLTYDEPLS HLIYAGSDFI LVPSIFEPCG LTQLVAMRYG TIPIVRKTGG LFDTVFDVDN | |
| DKERARDRGL EPNGFSFDGA DSNGVDYALN RAISAWFDAR SWFHSLCKRV MEQDWSWNRP ALDYIELYRS | |
| ASKL | |
| B. GBSS (61-300) + 551 (400-622) | |
| RRGGRFPSLV VCASAGMNVV FVGAEMAPWS KTGGLGDVLG GLPPAMAANG HRVMVVSPRY DQYKDAWDTS | SEQ ID NO:31 |
| VVSEIKMGDG YETVRFFHCY KRGVDRVFVD HPLFLERVWG KTEEKIYGPV AGTDYRDNQL RFSLLCQAAL | |
| EAPRILSLNN NPYFSGPYGE DVVFVCNDWH TGPLSCYLKS NYQSHGIYRD AKTAFCIHNI SYQGRFAFSD | |
| YPELNLPERF KSSFDFIDGY EKPVEGRKIN KATTVSNTYS KEVSGHGAIV PHLGKFYGIL NGIDPDIWDP | |
| NGIDINDWNP ATDKCIPCHY SVDDLSGKAK CKGALQKELG LPIRPDVPLI GFIGRLDYQK GIDLIQLIIP | |
| DLMREDVQFV MLGSGDPELE DWMRSTESIF KDKFRGWVGF SVPVSHRITA GCDILLMPSR FEPCGLNQLY | |
| AMQYGTVPVV HATGGLRDTV ENFNPFGENG EQGTGWAFAP LTTENMFVDI ANCNIYIQGT QVLLGRANEA | |
| RHVKRLHVGP CR | |
| C. GBSS (61-300) + SSIIa (481-732) | |
| RRGGRFPSLV VCASAGMNVV FVGAEMAPWS KTGGLGDVLG GLPPAMAANG HRVMVVSPRY DQYKDAWDTS | SEQ ID NO:32 |
| VVSEIKMGDG YETVRFFHCY KRGVDRVFVD HPLFLERVWG KTEEKIYGPV AGTDYRDNQL RFSLLCQAAL | |
| EAPRILSLNN NPYFSGPYGE DVVFVCNDWH TGPLSCYLKS NYQSHGIYRD AKTAFCIHNI SYQGRFAFSD | |
| YPELNLPERF KSSFDFIDGY EKPVEGRKIN DIIRSNDWKI NGIVNGIDHQ EWNPKVDVHL RSDGYTNYSL | |
| ETLDAGKRQC KAALQRELGL EVRDDVPLLG FIGRLDGQKG VDIIGDAMPW IAGQDVQLVM LGTGRADLER | |
| MLQHLEREHP NKVRGWVGFS VPMAHRITAG ADVLVMPSRF EPCGLNQLYA MAYGTVPVVH AVGGLRDTVA | |
| PFDPFGDAGL GWTFDRAEAN KLIEALRHCL DTYRKYGESW KSLQARGMSQ DLSWDHAAEL YEDVLVKAKY | |
| QW | |
| D. GBSS (61-300) + SSIIb (481-698) | |
| RRGGRFPSLV VCASAGMNVV FVGAEMAPWS KTGGLGDVLG GLPPAMAANG HRVMVVSPRY DQYKDAWDTS | SEQ ID NO:33 |
| VVSEIKMGDG YETVRFFHCY KRGVDRVFVD HPLFLERVWG KTEEKIYGPV AGTDYRDNQL RFSLLCQAAL | |
| EAPRILSLNN NPYFSGPYGE DVVFVCNDWH TGPLSCYLKS NYQSHGIYRD AKTAFCIHNI SYQGRFAFSD | |
| YPELNLPERF KSSFDFIDGY EKPVEGRKIN YTNYTFETLD TGKRQCKAAL QRQLGLQVRD DVPLIGFIGR | |
| LDHQKGVDII ADAIHWIAGQ DVQLVMLGTG RADLEDMLRR FESENSDKVR AWVGFSVPLA HRITAGADIL | |
| LMPSRFEPCG LNQLYAMAYG TVPVVHAVGG LRDTVAPFDP FNDTGLGWTF DRAEANRMID ALSHCLTTYR | |
| NYKESWRACR ARGMAEDLSW DHAAVLYEDV LVKAKYQW | |

### SUMMARY OF THE INVENTION

The invention provides the polypeptide sequence of GBSS enzymes (FIGS. 9A & 9B) that will enable the fused polypeptides of other starch synthases to become entrapped in the starch granules and be functional. The present invention provides modified starches with altered glucan chain lengths and a variety of starch synthase polypeptide domain fusions (TABLE V) to produce the same, as well as gene constructs that encode such fusions and in methods for the transformation of plants using such constructs as well as in the transformed plants thus obtained. The present invention also relates to the expression in plants of polypeptides-including SS enzymes as fusion proteins with improved affinity to starch and modified catalytic capabilities and to the in vivo and in vitro synthesis of glucan chains of modified lengths as compared to a plant producing native starch or starch produced with native starch synthases. In particular, the invention relates to the expression in plants of soluble starch synthase protein domains and/or polypeptide domains as fusion peptides with starch association domain of GBSS or SSI or any other SS enzyme. According to this invention, GBSS is any fusion protein thus generated using GBSS, for example, any SS or any other enzyme domain plus GLASS domains of GBSS and may include GLYTR domain as well. SS or Starch synthase means any starch synthesis enzyme that is present in soluble form, for eg. SSI, SSIIa, SSIIb, and SSIII.

The present invention provides a method for obtaining transformed plants that produce starches with modified glucan chain lengths. A farther object of this invention is to express the desired starch synthases or polypeptides in plants with modified functionalities in vivo and in association with starch or starch granules in order to introduce a desired modification in the average chain length of amylopectin.

The above objects are achieved by expressing a desired fusion protein of starch synthase or polypeptide that can interact with starch or starch granule in bringing a modification of glucan chain lengths.

By “interact with starch or starch granules” is generally meant that the fusion protein of starch synthases can modify, alter the chain length distribution of starch or modify the fine structure of starch. This interaction will result in starch or starch granules that differ from the naturally occurring plant starch in at least one property thereof, for example, glucan chain lengths, glucan composition, crystallinity, branching degree etc. Therefore, starch synthase fusion protein will influence at least one physical or chemical property of the starch.

Broadly, the invention relates to a method for expressing fusion proteins consisting of a desired one or more catalytic domains (“GLYTR” Domain) of one or more starch synthase or any other enzyme in association with glucan association domain (“GLASS” Domain) of GBSS or similar enzyme.

In addition, the invention also relates to a method for expressing fusion proteins consisting of a desired one or more catalytic domains (“GLYTR” Domain) of one or more starch synthase or any other enzyme in association with glucan association domain (“GLASS” Domain) of SSI or other similar enzymes.

The invention also relates to a method for expressing fusion proteins consisting of a desired domain (“GLASS” Domain) of any starch synthase enzyme from any organism fused with another desired domain of another starch synthase enzyme (“GLYTR” Domain) from the same or any other organism in any combination and vice versa.

The starch synthase protein domains or polypeptides expressed via the method of invention can be from any plant or from any plant part including seeds, leaves, roots, tubers, stems, stalks, fruits, and/or flowers. The starch synthase polypeptides thus expressed may or may not by themselves have natural affinity for starch or starch granules, and the method of the invention is used to provide a polypeptide of GBSS or SSI with such affinity.

Furthermore, the starch synthase polypeptides thus expressed may not by themselves have the natural affinity for starch or starch granules, and the method of invention is used to provide a polypeptide of SSI with such affinity.

The transformants of the invention expressing the starch synthase fusion proteins, may change the starch structure in different forms. For example, the starch synthases of the invention can change any one or the more of crystallinity of said starch, can change the glucan content, degree of branching, and especially the length of glucan chains in the amylopectin molecule.

The above modification in the glucan chain length distribution can bring changes in the affinity of the starch synthase enzymes that are intrinsic to the starch granule. And the change can increase or decrease the association of intrinsic starch synthase enzymes, like SSI and GBSS, to the starch granules.

Therefore, a further aspect of the invention relates to a method for providing a recombinant protein or polypeptide with affinity for starch granules and that has catalytic activity in order to bring changes in the structure of the starch.

The genes encoding the desired starch synthase polypeptide sequence may be derived from any source, including plants, animals, fungi, algae, yeasts, bacteria, and any other microorganisms. The expressed genes may be homologous or heterologous to the starch producing plant in which the fusion peptides of starch synthase are expressed.

A further aspect of the invention is that the genes encoding any of the starch synthase fusion polypeptides can be variants or mutants of such proteins, such as those known in the art and/or obtainable via genetic manipulations. This includes mutant enzymes with biological activity but, with altered properties in terms of altered substrate binding activity, altered substrate specificity, and finally altered kinetic properties.

In another aspect the present invention provides expression of fusion proteins with of the invention is that the expression of fusion proteins with the starch association domain of SSI and/or GBSS (“GLASS” Domain) which may include partial or full length catalytic domains of any starch synthases, starch branching enzymes, debranching enzymes, disproportionating enzymes, kinases, phosphorylases and any of the isoforms of above enzymes.

More in particular, the expression of these starch synthase fusion proteins along with the starch association domain of GBSS will lead to a “modified-starch”, the subject matter of invention.

The said modified starch may be further modified according to the techniques known to the skilled person. Whether in modified or unmodified form, the starch will be used for food and non-foodstuff.

The above “modified starch” resulting from the expression of fusion proteins of starch synthases will have at least one of the listed below altered or improved properties as compared to the natively produced starch by a plant. The modified starch will have an altered or improved morphology, retrogradation, waterbinding or swelling potential of the granules, gel strength, adhesiveness, cohesiveness, hardness, elasticity, increased or decreased granule size, degree of branching, crystallinity, degree of cross-linking, and increased or decreased glucan chain lengths.

The present invention further provides the following method of:

a) providing a genetic construct containing at least one or more nucleotide sequence encoding desired polypeptide sequence containing one or more catalytic domains (“GLYTR” Domain) of starch synthase fusion protein combined with at least one nucleotide sequence encoding starch association domain (“GLASS” Domain) of GBSS or SSI;

b) providing a genetic construct containing at least one or more nucleotide sequence encoding desired polypeptide sequence(s) containing at least one domain (“GLYTR”or “GLASS” Domain) of one starch synthase with at least one nucleotide sequence encoding desired polypeptide sequence of another domain (“GLYTR”or “GLASS” Domain) of another starch synthase and vice versa;

c) transforming a plant with any of the above construct(s); and

d) expressing the genetic construct in the plant in vivo.

The present invention provides an isolated DNA molecule encoding a fusion protein consisting of four different functional domains selected from the group consisting of GLASS, LINKR, GLYTR, and CTEND which are operably linked to one another. The isolated DNA molecule of the present invention may contain, for example, a GLASS domain which contains a GBSS GLASS. Further, the GBSS GLASS of the present invention may contain a GLASS of SEQ ID NO: 1. Alternatively, the isolated DNA molecule of the present invention may contain a GLASS domain which contains a SSI GLASS. The SSI GLASS of the present invention may contain, for example, a GLASS of SEQ ID NO: 2. Moreover, the isolated DNA molecule of the present invention may contain a GLASS domain which contains a SSII GLASS. For example, the SSII GLASS of the present invention may ontain a GLASS of SEQ ID NOs: 3 and/or 4. Furthermore, the isolated DNA molecule of the present invention may contain a GLASS domain which contains a SSIII GLASS. The SSIII GLASS of the present invention may further contain a GLASS of SEQ ID NO:5

In one embodiment, the isolated DNA molecule of the present invention contains at least one of a GBSS GLASS, a SSI-GLASS, a SSII-GLASS or a SSII-GLASS wherein the GLASS or GLASS domain are a GLASS or GLASS domain of a glucan producing organism or at least 80% (preferably at least 85%, more preferably at least 90%, alternatively at least 95%, or at least 98%) identical or similar to a GLASS or GLASS domain peptide of a glucan producing organism.

The present invention further provides an isolated DNA molecule, as described herein and above wherein the LINKR domain is a GBSS-LINKR, a SSI-LINKR, a SSII-LINKR and/or a SSIII-LINKR. The LINKR of the invention may contain a LINKR sequence containing any of SEQ ID NOs:121-171; 336-386;527-577; 733-783; and/or 983-1033.

The present invention further provides an isolated DNA molecule, as described herein wherein the GLYTR domain may contain a GBSS-GLYTR, a SSI-GLYTR, a SSII-GLYTR and/or a SSIII-GLYTR. More specifically, the GLYTR domain of the present invention may contain a GLYTR sequence containing at least one of SEQ ID NOs:1136, 1137, 1138, 1139, and/or 1140. Alternatively, the GLYTR of the present invention may contain a GLYTR sequence containing at least one of SEQ ID NOs: 172-222; 387-437; 578-628; 784-834; and/or 1034-1084.

The present invention further provides an isolated DNA molecule, as described herein and above wherein the CTEND domain is a GBSS-CTEND, a SSI-CTEND, a SSII-CTEND and/or a SSIII-CTEND. The CTEND of the invention may contain a CTEND sequence containing any of SEQ ID NOs: 1146, 1147, 1148, 1148, 1149 and/or 1150. Alternatively, the CTEND of the present invention may contain a CTEND sequence containing a CTEND sequence containing at least one of SEQ ID NOs:223-266; 438-461; 629-676; 835-882; and/or 1085-1135.

The present invention provides an isolated DNA molecule encoding a fusion peptide which contains a GBSS GLASS domain operably linked to a LINKR and a catalytic domain from a functional protein that synthesizes an ∀-1,4 glucan or an ∀-1,3 glucan, or an ∀-1,6 glucan, wherein the fusion peptide is capable of modifying the glucan structure of a starch producing organism when starch is produced by such an organism or part thereof in the presence of a fusion peptide of the present invention.

In a further embodiment, the present invention provides a DNA molecule which encodes a fusion peptide, and a fusion peptide coded for by the same, wherein the GLASS and/or LINKR sequence contained therein contains at least one of SEQ ID NOs:75-120; 284-335, 475-526; 682-732; 933-982 and/or 121-171, 336-386, 527-577, 733-783, and 983-1033.

In a further embodiment, the present invention provides an isolated DNA molecule encoding a polypeptide, and a polypeptide so encoded, with glucan association properties of a maize GBSS enzyme, and being capable of modification of starch metabolism in a plant or plant cell, the DNA containing a molecule of, for example, at least one of the following:

(a) a DNA molecule encoding a protein domain having the amino acid SEQ ID NO:1;

(b) a DNA molecule containing a corresponding nucleotide sequence from SEQ ID No:1141;

(c) a DNA molecule containing a nucleotide sequence differing from the sequence of the DNA molecules of (a) or (b) due to the degeneracy of the genetic code;

(d) a DNA molecule containing a DNA sequence which hybridizes to any one of the DNA molecules of (a), (b) or (c) or fragment thereof, and which is equal to or more than 80% homologous or identical or similar to the DNA molecule of (a), (b), or (c), or fragment thereof, wherein the DNA sequence encodes a polypeptide with Glucan Association Domain (Domain A) of a GBSS enzyme.

The present invention provides an isolated DNA molecule encoding a polypeptide with a glycosyl transferase function of a soluble or granule bound maize SS enzymes capable of modifying starch metabolism in a plant or plant cell, the DNA molecule containing, for example, at least one of the following:

(a) a DNA molecule encoding a protein domain containing an amino acid of SEQ ID NOs:1, 2, 3, 4, and 149;

(b) a DNA molecule containing the corresponding nucleotide sequence of SEQ ID NOs: 1141, 11142, 1143, 1144, and 1145;

(c) a DNA molecule containing a nucleotide sequence differing from the sequence of the DNA molecules of (a) or (b) due to the degeneracy of the genetic code,

(d) a DNA molecule containing a DNA sequence which hybridizes to any one of the DNA molecules of (a), (b) or (c), or fragment thereof, and which is equal to or more than 80% homologous or identical to the DNA molecule of (a), (b), or (c), or fragment thereof, wherein the DNA sequence encodes a polypeptide with a glycosyl transferase domain of a SS enzyme.

The present invention provides a recombinant or isolated DNA molecule, as described, containing a maize GBSS nucleotide coding region encoding for an amino acid sequence of SEQ ID NOs: 101-146 fused with a corresponding coding region of a maize SS enzyme that encode for an amino acid sequence containing any of SEQ ID NOs: 35-74; 121-171; 172-222; 223-266; 268-283; 284-335; 336-386; 387-437; 438-461; 463-474; 475-526; 527-577; 578-628; 629-676; 678-681; 682-732; 733-834; 835-882; 884-932; 933-982; 1034-1084; and/or 1085-1135.

The present invention provides a recombinant or isolated DNA molecule, as described, containing a GLYTR, LINKER or CTEND domain DNA sequence containing any of SEQ ID NOs: 172-222; 387-437; 578-628; 784-834; 1034-1084, 121-171; 336-386; 527-577; 733-783; 983-1033 or 223-266; 438-461; 629-676; 835-882; 1085-1135 operably linked in any order with a corresponding DNA sequence that encodes for a glucan association domain containing any of SEQ ID NOs: 75-120; 284-335; 475-526; 682-732; and/or 933-982.

The present invention provides a recombinant or isolated DNA molecule, as described, further containing a DNA sequence differing from the sequence of any of the DNA molecules of SEQ ID NOs: 34-1150 due to the degeneracy of the genetic code, and/or protein or polypeptide originating from a different source, such as a plant species other than plant species such as maize, bacteria (e.g. E. Coli), Yeast, algae (Chlamydomonas), or fungus.

The present invention provides a recombinant or isolated DNA molecule, as described herein, wherein the DNA sequence contains at least one coding region of a glucan association domain of SEQ ID NOs:75-120; 284-335; 475-526; 682-732; and/or 933-982 fused with a coding region of any glucan transferases listed in Table XXXVII.

The present invention further provides a method of expressing a starch synthase fusion proteins or polypeptides in a plant, in which the starch synthase protein or polypeptide domains are expressed as a fusion with a glucan association domain of granule bound starch synthase. Theprotein or polypeptide of the method of the invention may be heterologous with respect to the plant in which the fusion is expressed.

The present invention further provides a method, as described herein, wherein th method involves the steps of:

providing a genetic construct containing at least one nucleotide sequence encoding the desired protein domain or polypeptide domain combined with at least one nucleotide sequence encoding a glucan association domain of GBSS, so that the construct encodes a fusion of the desired protein/polypeptide and at least one glucan association domain;

transforming a plant with the genetic construct;

expressing the genetic construct in the plant.

The present invention further provides a method, as described herein, in which the protein or polypeptide or recombinant protein or recombinant polypeptide is an enzyme. Such an enzyme of the present invention may, for example, be an enzyme which is an enzyme that can interact and associate with starch or starch granules, or facilitate or be entrapped in starch or starch granules, and is capable of at least one of modifying, increasing, decreasing, altering or influencing starch structure or starch synthesis.

The present invention further provides a vector containing a DNA molecule as provided herein. The vectors of the present invention may contain, for example, a DNA molecule which is linked in the sense orientation to DNA elements ensuring transcription of a translatable RNA in a prokaryotic or an eukaryotic cell.

The present invention further provides a host cell containing a vector of the present invention.

In a further embodiment, the present invention provide a plant cell containing a DNA molecule of the present invention linked to a heterologous promoter.

The present invention further provides a plant containing a plant cell of the present invention. The plants according to the present invention may be, for example, a cereal, such as maize, rice, wheat, barley, oats, or a root crop, such as potato, sweet potato, cassaya, yam, taro, or other starch producing plant, such as peas or banana. Moreover, the plants of the present invention may contain or produce starch or starch granules in at least one of its parts, including its seeds, leaves, roots (tubers), tubers, stems, stalks, fruits, grains or flowers. Furthermore, the plants of the present invention include elements containing a homologous or heterologous promoter specific for expression of said DNA molecule in the at least one of its parts.

The present invention provides seeds from the plant of the present invention, which are preferably capable of expressing the recombinant molecule or DNA molecule of the present invention.

The present invention provides amodified starch derived from cells of a plant or plant part of the present invention.

In a further embodiment, the present invention provides a food or feed containing a modified starch of the present invention or plant or plant part of the present invention.

### BRIEF DESCRIPTION OF THE FIGURES

FIG. 1. Shows ¹⁴C-ADPG incorporation as dpms (disintegrations per minute) into different glucan chain lengths separated on Sepharose CL-6B Column by various starch synthase enzymes from maize. The glucan (potato amylopectin and glycogen) was debranched after SS enzyme reaction and prior to running on the column. Shown in the order (from top to bottom of the figure) are, GBSS (granule bound starch synthase), Du-1 (SSIII), soluble starch synthase IIa, soluble starch synthase IIb (IIb), and soluble starch synthase I (SSI). Dp=degree of polymerization or number of glucose units. There is a significant difference in the chain length specificity of various enzymes. For example, GBSS incorporated most of the ¹⁴C-ADPG into very long glucan chains that are more than 30 units long. Du-I or SSIII incorporated more than half the label into glucan chains that are in between dp 20 and 30. SSIIa and SSIIb incorporated most of the ¹⁴C-ADPG into glucan chains that are shorter than 20. Most of the ¹⁴C-ADPG incorporation by SSI was into the glucan chains that are shorter than dp 10. Therefore, there are four distinct classes of starch synthases with differences in chain length specificity that are detected in maize endosperm.

FIG. 2. Shows results from Sepharose CL-6B chromatography of debranched products of GBSS and SSI. The figure displays clear distinction in the chain length specificities of GBSS and SSI enzymes in that ¹⁴C-labeled products of GBSS elute much earlier than the ¹⁴C labeled products of SSI. This means that GBSS elongates longer glucan chains, where as SSI elongates shorter glucan chains.

FIG. 3. Shows results from thin layer chromatography of debranched glycogen after ¹⁴C-ADPG incorporation into various glucan chains by different starch synthase enzymes in maize. Panel on the left shows the carbohydrate staining and panel on the right shows ¹⁴C-label incorporation into different glucan chain lengths. Carbohydrate staining shows that there is equal amount of carbohydrate loaded in each well. Also, there is equal amount of carbohydrate visible in each glucan class. However, panel on the right shows that each enzyme picked a different glucan class for ¹⁴C-ADPG incorporation. The numbers on the left indicate the size of the glucan in each class. Maltooligosaccharide (MOS) ladder (of known sizes) as a marker was run in order to enable us to estimate the glucan chains in each lane. The numbers 1-7 on the left panel indicate the number of glucoses. The numbers on the far left indicate the glucan chain (8-13) lengths interpreted based on the MOS ladder. The right panel shows that GBSS and Du-1 incorporated ¹⁴C-ADPG mostly into glucan chains longer than dp 13. Contrarily, SSI incorporated most of the ¹⁴C-ADPG into glucan chains that are dp 8 or 9. SSIIa incorporated most of the ¹⁴C-ADPG into glucan chains that are dp 8. SSIIb incorporated most of the ¹⁴C-ADPG label into glucan chains that are dp 11 and 12. Therefore, there appears to be a chain length specificity for each SS enzyme.

FIG. 4. Shows SDS-Page of proteins associated with the starch granules of maize kernels. Proteins from starch granules were extracted by boiling and ran on 10% polyacrylamide gels. Proteins were stained with coomassie blue. The figure shows that majority of the protein entrapped in the starch granules is GBSS and there is some SSI and branching enzymes as well.

FIG. 5. Shows proposed model for starch synthases based on 3D-PSSM automated fold recognition technique (Kelley et al., 2000). All the five known starch synthases from maize have two distinct domains with a linker in between. The labels on the top show the corresponding names of these domains based on the functionality disclosed in the present application. “GLASS” stands for glucan association domain and “GLYTR” stands for glycosyl transferase domain. “GLASS” and “GLYTR” are linked to each other by “LINKR” sequence.

GBSS is shown in FIGS. 5A-1, upper left panels.

SS1 is shown in FIGS. 5A-2, upper right panels.

SSIIa is shown in FIGS. 5A-3, lower left panels.

SSIIb is shown in FIGS. 5A-4, lower right panels.

DU1 is shown in FIGS. 5B-1 (FIG. 5. Cont.d).

FIG. 6. Is a cartoon showing the location of Glycosyl transferase group 1(Pfam 00534) domain of maize starch synthases.

FIG. 7. Shows a picture of affinity gel electrophoresis to determine glucan association peptide of GBSS.

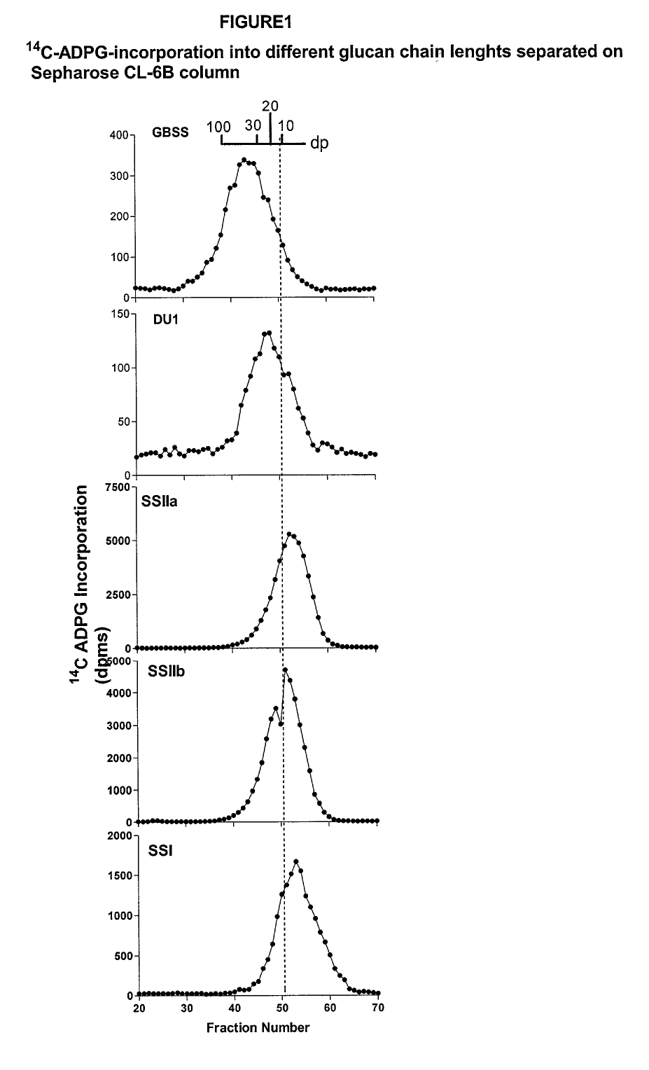

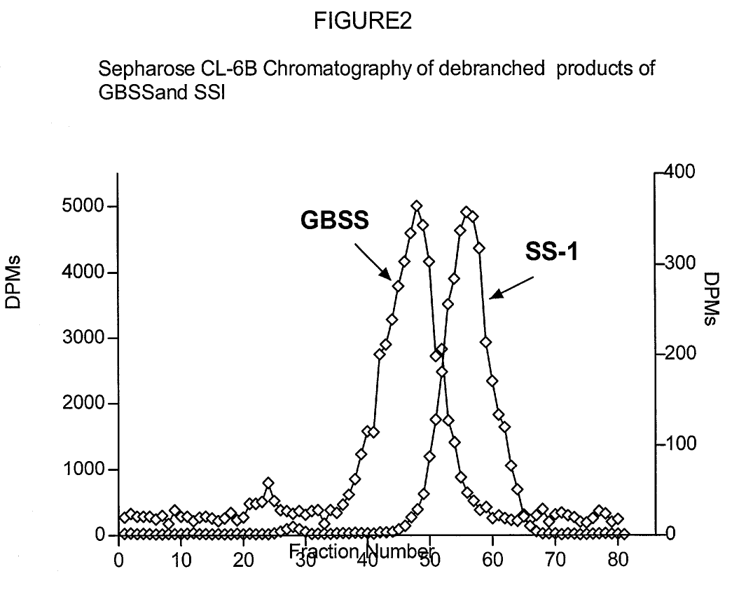

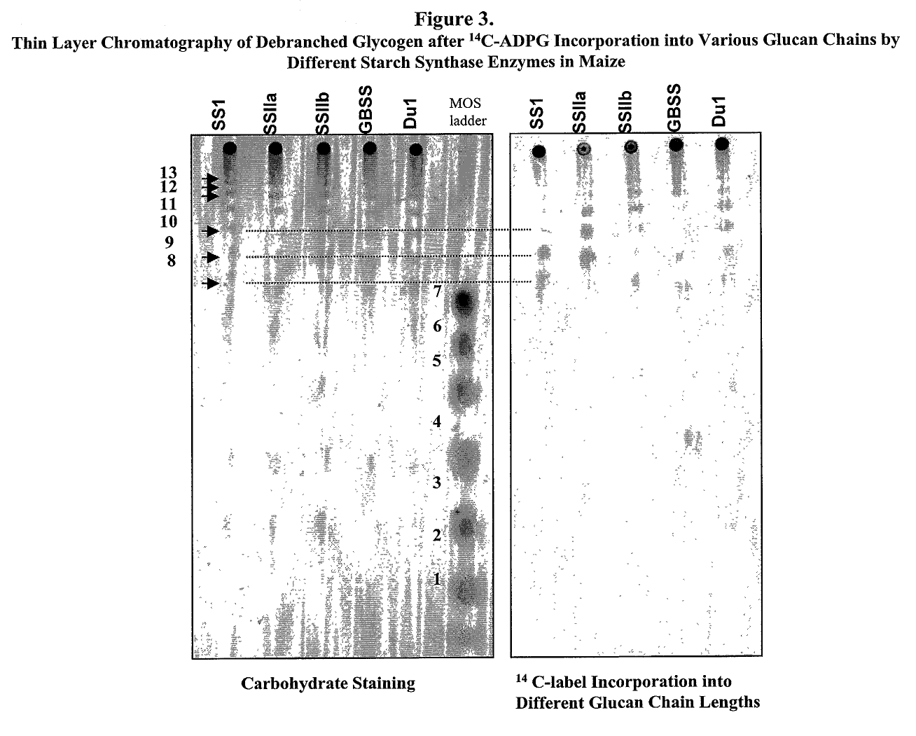

Panel 1=Native gel containing 0.2% potato amylopectin. It shows GBSS has strong affinity to amylopectin.

Panel 2=GBSS enzyme was digested into various peptides using Endo-Glu-C or V8 enzyme. The peptides were separated on 10% SDS-PAGE gels and visualized by using silver staining of peptides.

Panel 3=Purified GBSS enzyme on 10% SDS-PAGE gels.

Panel 4=V8 enzyme peptides that were bound to amylopectin in the native gels were excised and ran on 10% SDS-gels. The arrows indicate the peptides that had affinity to glucan.

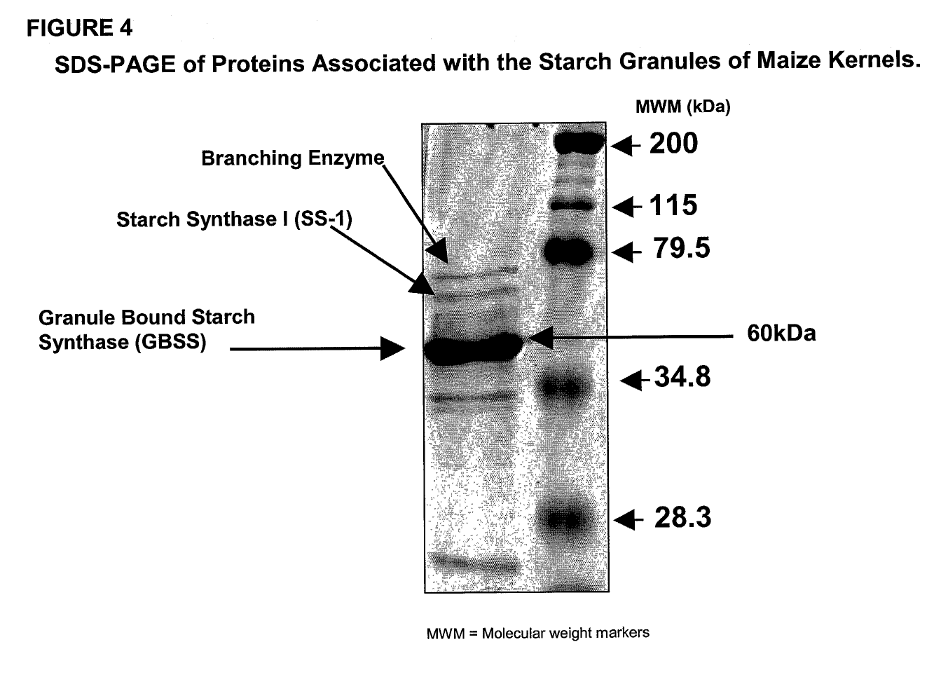

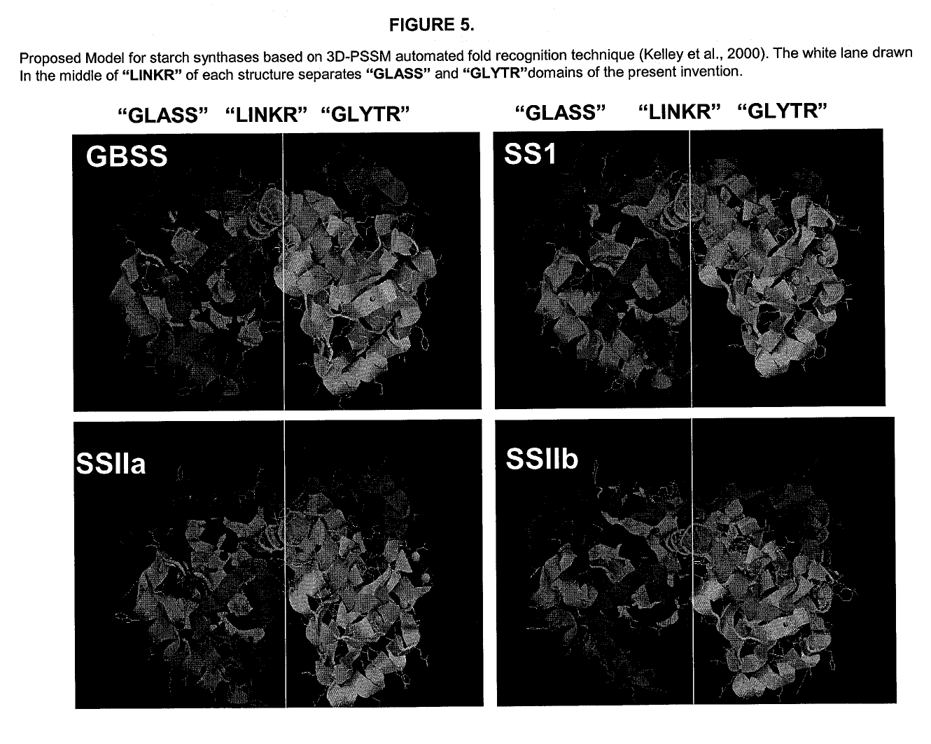

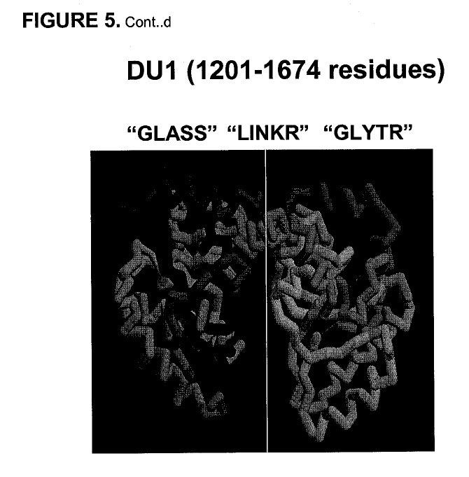

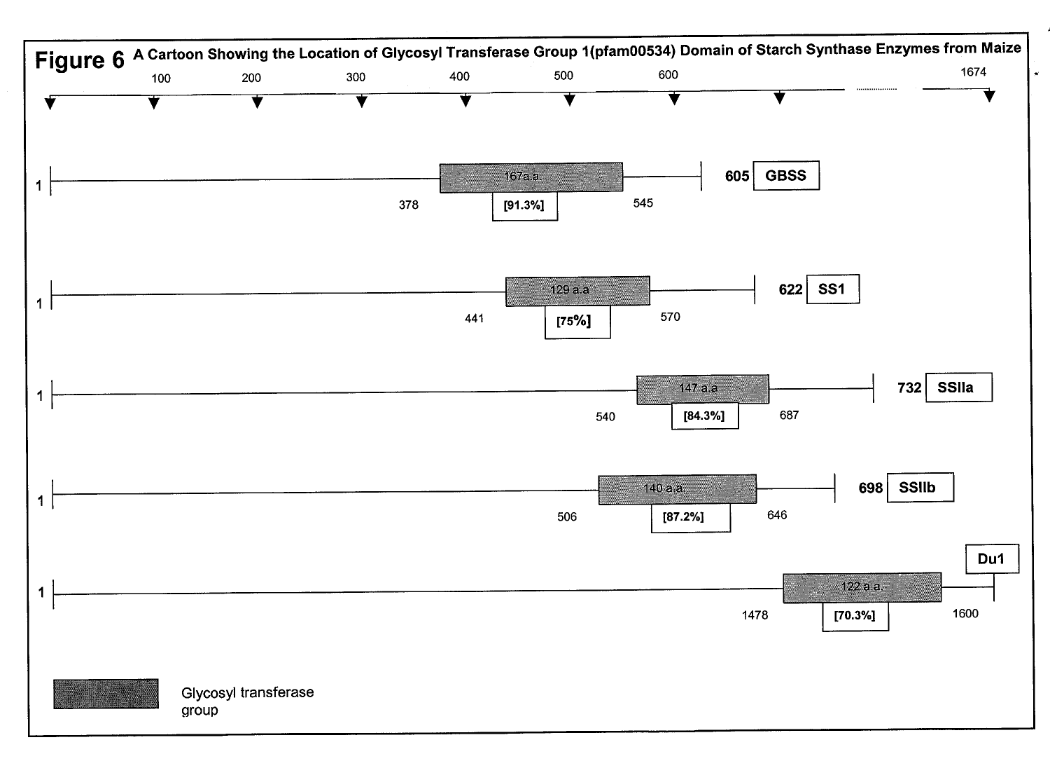

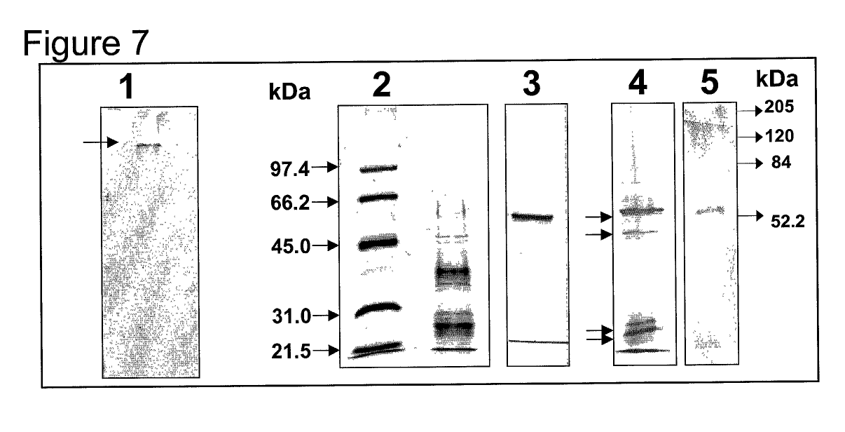

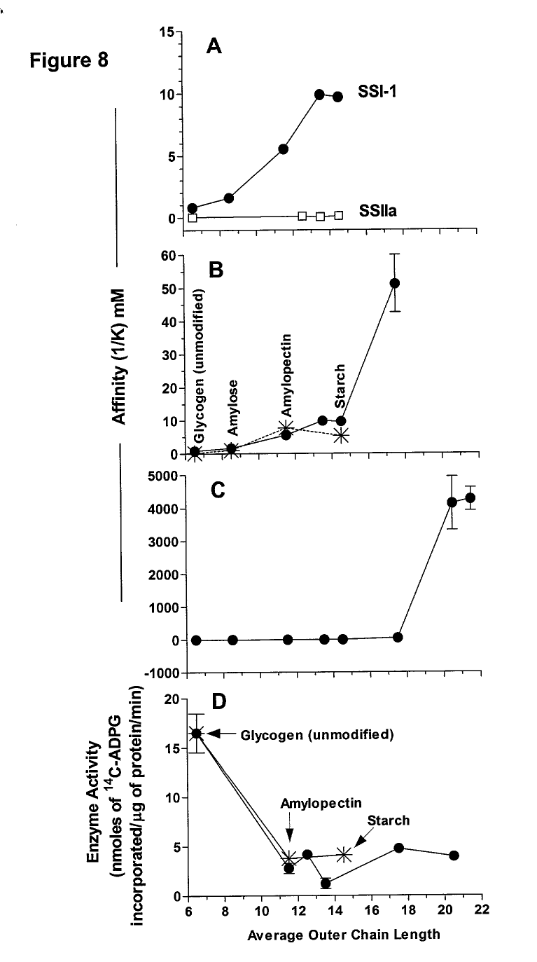

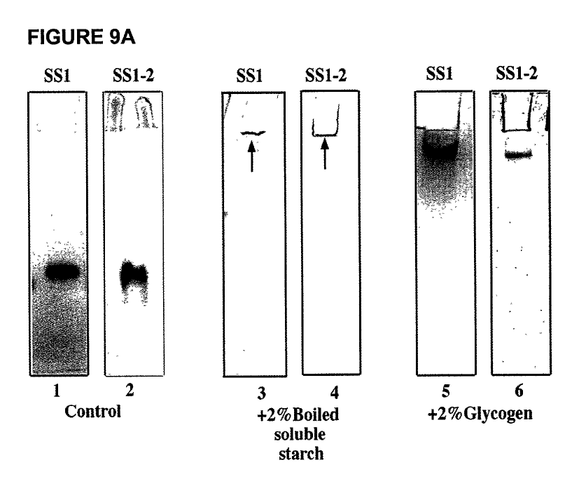

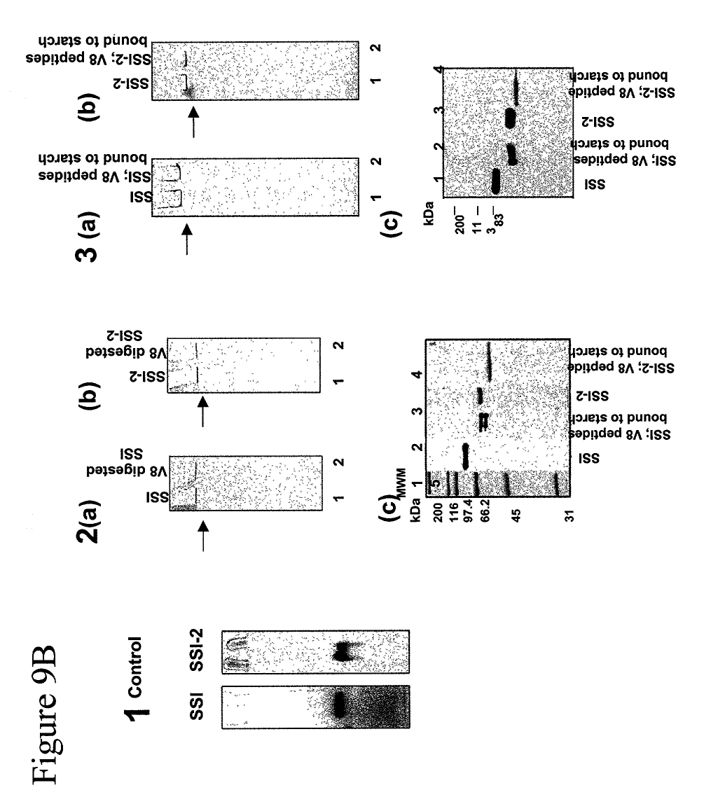

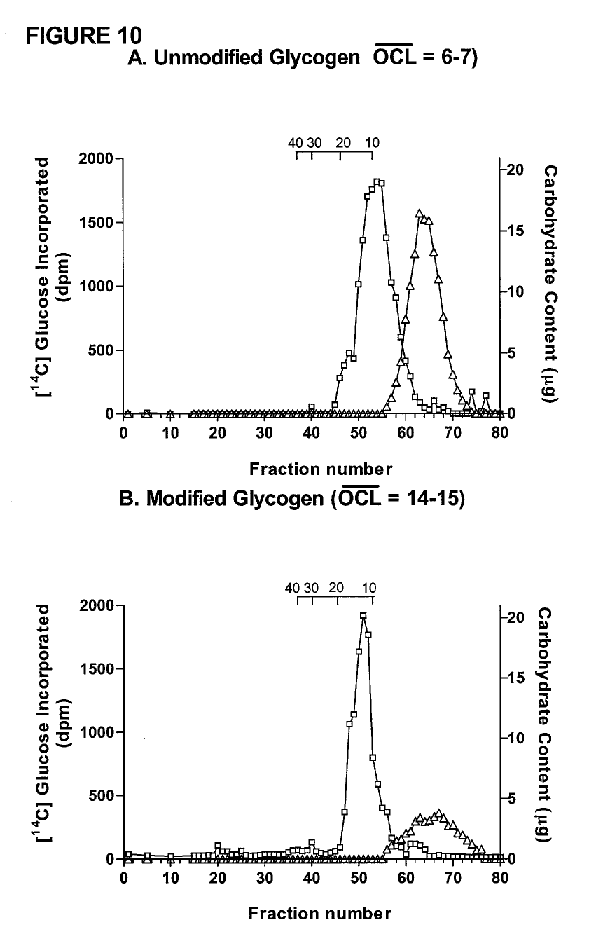

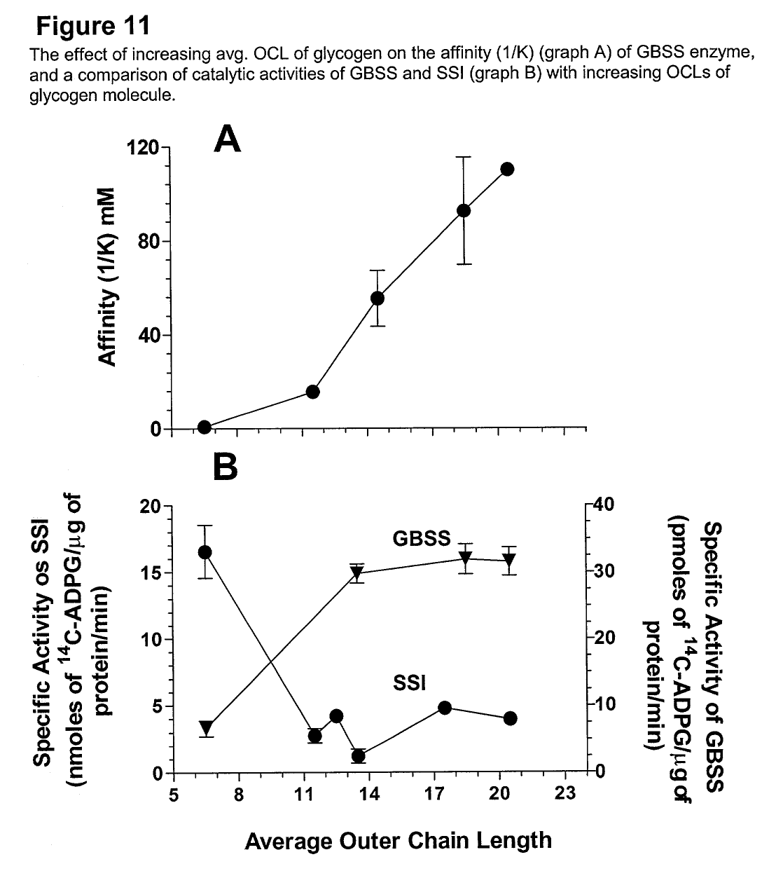

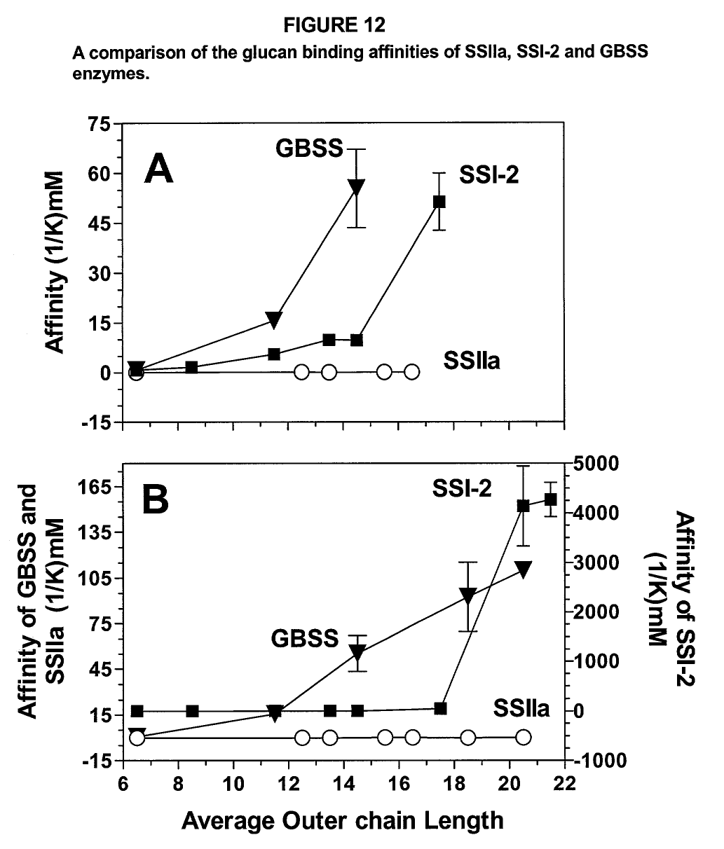

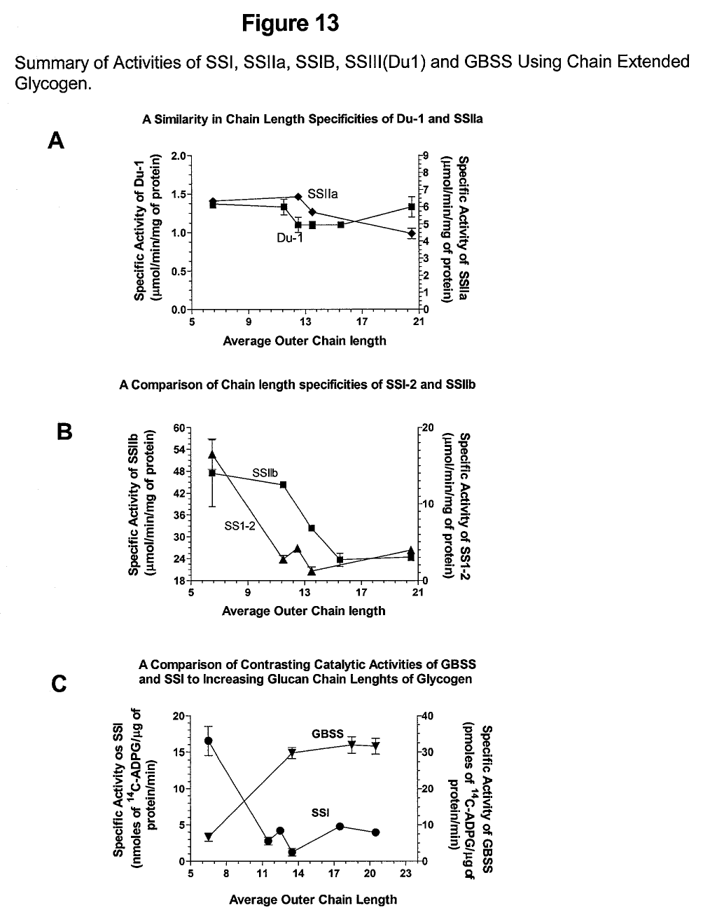

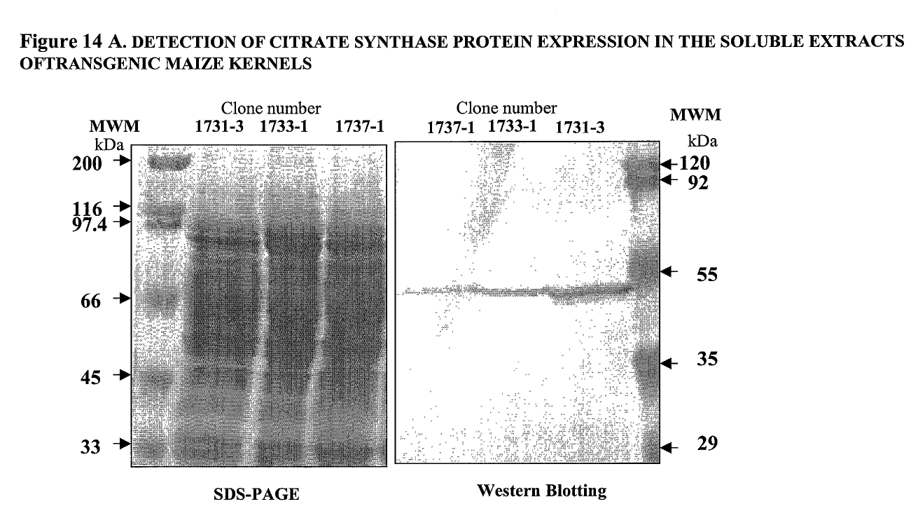

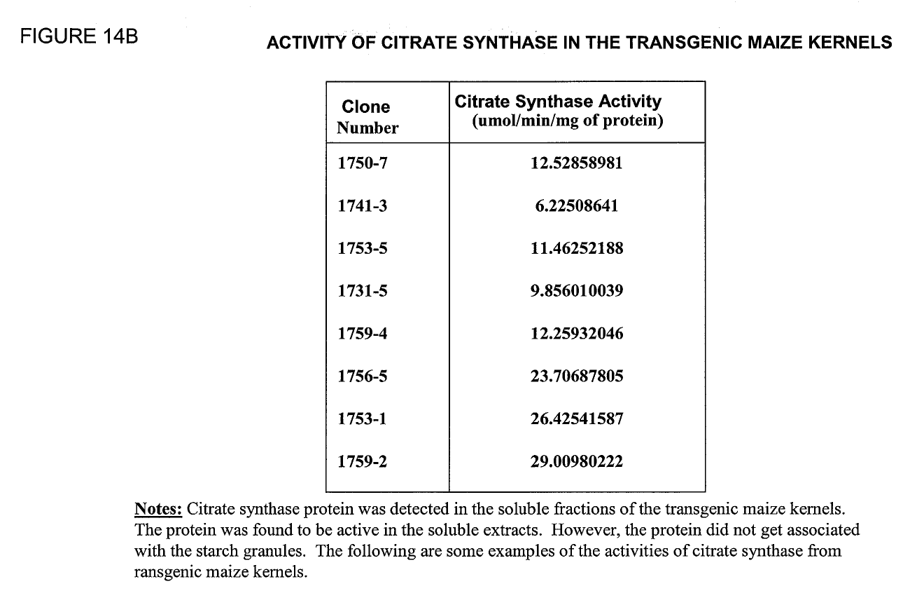

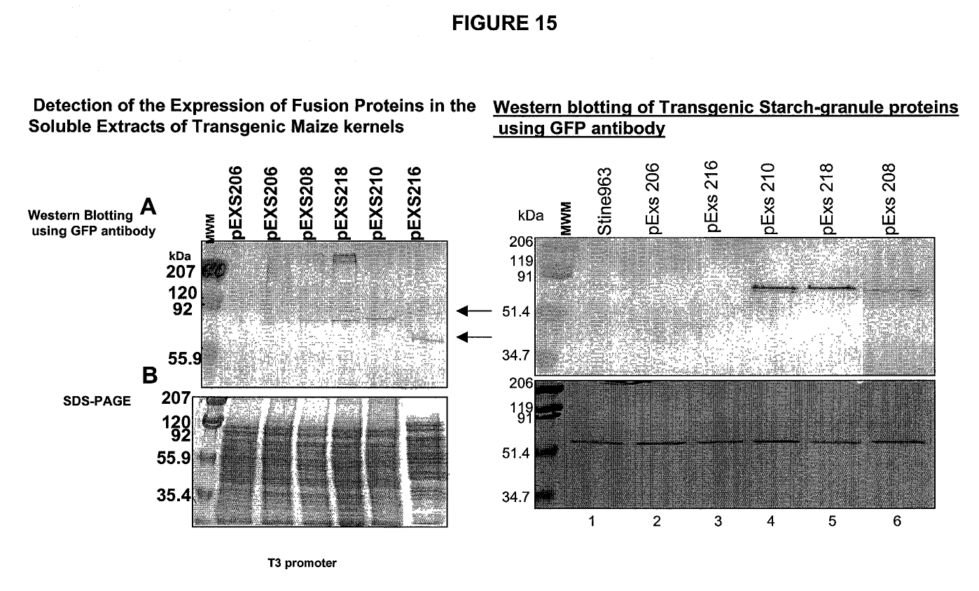

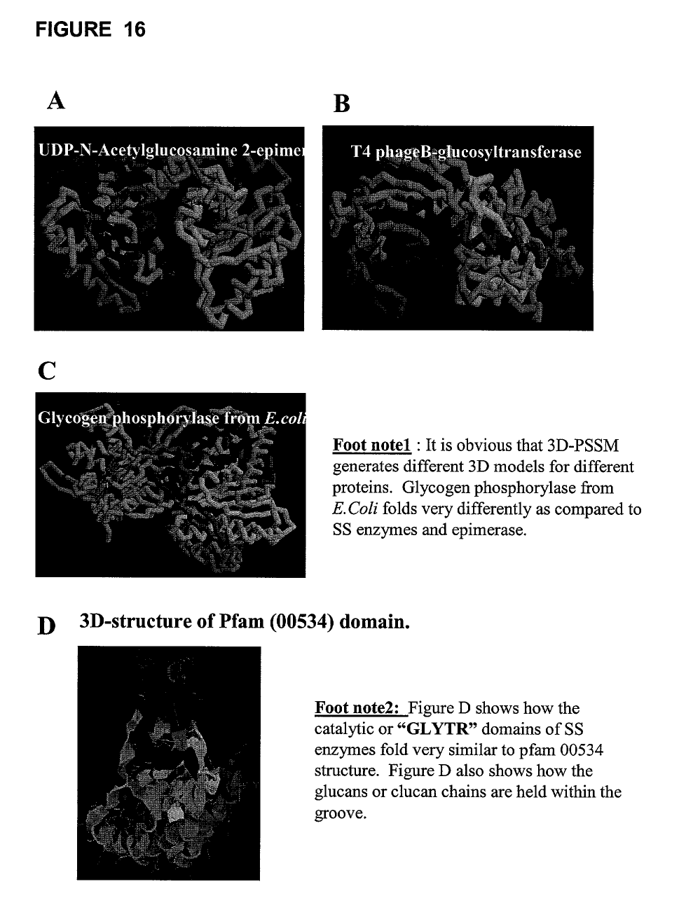

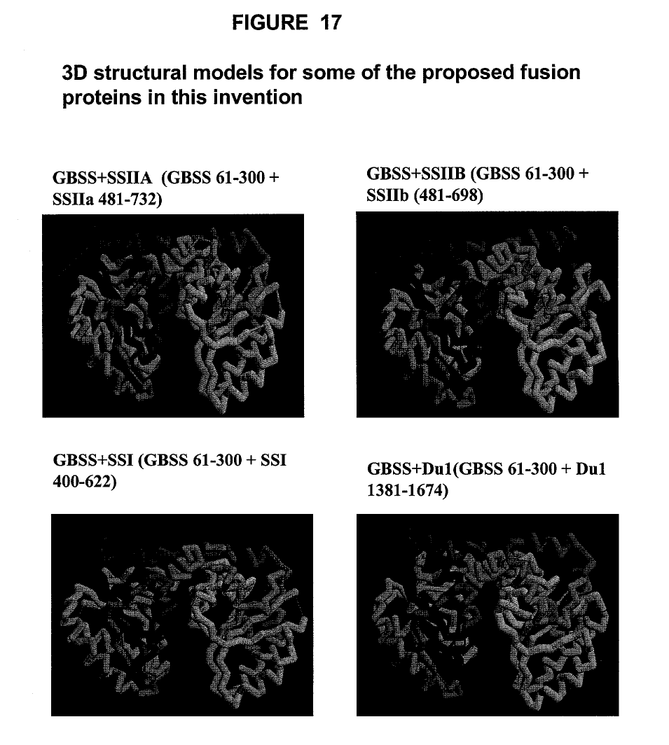

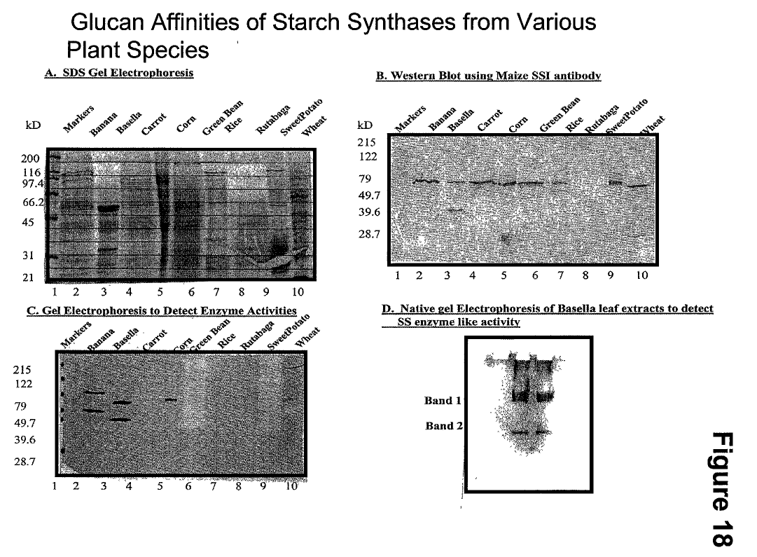

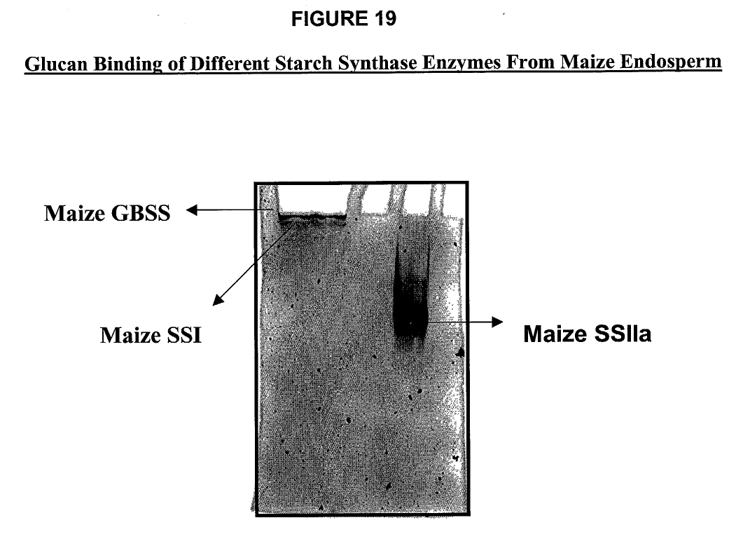

Panel 5=A renaturing gel for detecting the activity of SS enzymes. The smallest peptide from FIG. 4 of the above was blotted onto PVDF membrane. The amino acid sequence of the peptide is as follow.

KIYGPVAGTDYRDNQL RFSLLCQAAL EAPRILSLNN NPYFSGPYGE
DVVFVCNDWHTGPLSCYLKSNYQSHGIYRD AKTAFCIHNI
SYQGRFAFSD YPELNLPERF
KSSFDFIDGYEKPVEGRKINWMKAGILEAD RVLTVSPYYA EE

FIG. 8 shows the effect of increasing avg. OCL of glycogen on the affinity (1/Kd) (graphs, A, B, C) and catalytic activity (graph D) of the SSI-2 enzyme.

FIG. 8A shows the effect of increasing avg. OCL of glycogen on the affinity (1/Kd);

FIG. 8B shows the effect of increasing avg. OCL of glycogen on the affinity (1/Kd);

FIG. 8C shows the effect of increasing avg. OCL of glycogen on the affinity (l/Kd); and

FIG. 8D shows the catalytic activity of the SSI-2 enzyme. The graphs shows increased affinity and decreased enzyme activity with increase in the average outer chain length of the substrate molecule. Graph 3B (8B) shows how the affinities of amylose, amylopectin and starch fall within the same range of modified glycogens with extended OCL and also shows upward trend in affinity after dp of about 17. Graph 3D (8D) shows the contrasting results with the enzyme activity using modified glycogens with extended OCLs. Data are average of three separate replications±SE.

FIG. 9. Shows a comparison of mobility of SSI and SSI-2 proteins in the substrate containing native gels.

FIG. 9A.

SSI and SSI-2 proteins were run in the native gels containing either none (panels 1 and 2) or 2% starch (panels 3 and 4), or 2% glycogen (panels 5 and 6). The gels were stained for activity using I2 solution (for details, see materials and methods section). The arrows indicate the binding of protein to the substrate in the gels containing 2% starch.

FIG. 9B.

Panels 2A, 2B, and 2C are coomassie staining, and panels 3A, 3B, and 3C are activity staining of the above proteins. Panels 2A, 2B, 3A, and 3B are native gels containing 2% starch and panels 2C and 3C are renaturing gels (see materials and method section for details). The gels show V8 peptide(s) of SSI (2A, 3A, 2C, and 3C) and SSI-2 (2B, 3B, 2C and 3C) that were bound to the substrate in the native gels (2A, 3A, 2C), and were active (2B, 3B, 3C). The arrows indicate the protein or peptide(s) bound to the substrate in the gels right in the well itself. For panels, 2C and 3C, molecular weight markers were shown on the left in kD.

FIG. 10. Shows a comparison of the elution profile of ¹⁴C-labeled glucans on Sepharose CL-4B column.

FIG. 10A shows debranched products of SSI reaction using unmodified glycogen.

FIG. 10B shows the results with modified glycogen (OCL=14-15).

Both were run on a Sepharose CL-4B. At the end of the enzyme reaction, carbohydrate was subjected to Isoamylase digestion (as described in the materials and methods section). Open squares are elution of total carbohydrate as absorbance at 490 nm of fractions mixed with phenol and H₂SO₄; open triangles are the elution profile of ¹⁴C-labeled products. The scale bar for each graph shows profile of elution of corresponding chain lengths of debranched amylopectin ran on the same column. Each data point is an average of 3 separate runs on the columns.

FIG. 11.

FIG. 11A shows increased affinity and increased enzyme activity of GBSS with increase in the average outer chain length of the substrate molecule.

FIG. 11B shows the contrasting results with the enzyme activities of GBSS and SSI enzyme using modified glycogens with extended OCLs. Data are average of three separate replications±SE.

FIG. 12. Shows a comparison of the glucan binding affinities of SSIIa, SS1-2, and GBSS enzymes. Affinity is calculated based on the molar availability of outer chain lengths.

FIG. 12A shows increase in the affinity of GBSS and SSI-2 enzymes to increase in the outer chain length of modified glycogen up to dp ˜14 to 16. To the same increase in the outer chain lengths, SSIIa did not show any increase in the affinity.

FIG. 12B shows a linear increase in the affinity of GBSS to further increments in the chain length whereas, SSIIa did not show any increase in the affinity.

It is interesting to note that SSI-2 displayed more than 4000 fold increase in the affinity when the length of the outer chains was extended on an average up to 21glucose units.

FIG. 13. Shows summary of activities of SSI, SSIIa, SSIIb, SSIII (DuI) and GBSS using chain extended glycogen. Based on the observations in this figure, the present invention classifies maize a-1,4 glucan transferases or starch synthases based on their specificities to process various lengths of glucan chains in the amylopectin cluster. For example, according to the present invention, ‘Class I’ enzymes that include maize SSI and like enzymes, and preferentially elongate a-1,4 glucan chains to synthesize shorter A and BI chains; ‘Class II’ enzymes that include maize SSIIa and SSIIb and like enzymes, and preferentially add a glucose unit(s) to a-1,4 glucan chains to synthesize longer A and B 1 chains and intermediate B2 or B3 chains; ‘Class III’ enzymes that include maize SSIII and GBSS, and preferentially add a glucose unit(s) to a-1,4 glucan chains to synthesize longer A, B 1, B2 and B3 chains as well as longer B3 or C chains of amylopectin. In maize or any other crop when transformed to express or over express any one specific class of starch synthases described above will result in an increased number of glucan chains in that specific class.

FIG. 13A shown A similarity in Chain Length Specificities of Du-1 and SSIIa;

FIG. 13B shows A Comparison of Chain Length Specificities of SSI-2 and SSIIb;

FIG. 13C shows A Comparison of Contrasting Catalytic Activities of GBSS and SSI to Increasing Gluican Chain Lenghs of Glycogen.

FIG. 14.

FIG. 14A shows detection of the expression of fusion proteins in the soluble extracts of transgenic maize kernels. The transgenic proteins are expressed in the soluble extracts.

FIG. 14B shows detection of the transgenic fusion protein only in the 210 and 218 (See example number I for details).

FIG. 15.

FIG. 15A shows the detection of transgenic citrate synthase protein in the soluble extracts of maize kernels. However, the protein did not get associated with the starch granules.

FIG. 15B shows activities of citrate synthase from transgenic maize kernels.

FIG. 15C (right panel) shows Western blotting of Transgenic Starch-granule proteins using GFP antibody.

FIG. 16. Shows the differences in the models generated by 3D-PSSM for different proteins. Glycogen phosphorylase from E. Coli folds very differently as compared to SS enzymes and epimerase. It confirms that all SS enzymes have a similar 2 domain but functionally different 3D structures.

FIG. 16A shows UDP-N-Acetylglucosamine 2-epimer;

FIG. 16B shows T4 phage B-glycosyltransferase;

FIG. 16C shows Glycogen phosphorylase from E. coli.;

FIG. 16D shows how the catalytic or GLYTR domains of SS enzymes fold very similar to pfam 00534 structure. FIG. 16D also shows how the glucans or glucan chains are held within the groove.

FIG. 17. Shows 3D structures of some of the proposed fusion proteins.

FIG. 17A (upper left) shown GBSS+SSIIA;

FIG. 17B (upper right) shows GBSS+SSIIB;

FIG. 17C (lower left) shows GBSS+SSI; and

FIG. 17D (loer right) shows GBSS+DuI.

FIG. 18. Shows SDS-electrophoresis and coomassie staining of proteins from various plants, namely banana fruit, basella leaf, carrot root, maize endosperm, green bean pods, rice endosperm, rutabaga root, sweet potato root, and wheat endosperm. The proteins were run on native gel containing 2% boiled starch. The peptides or proteins that were bound to the glucan in the well were visualized by coomassie staining; were excised out of the native gel, and run on 10% SDS-gel. Very few peptides were bound to the glucan (data not shown). The proteins that were bound were transferred onto a nitrocellulose membrane for performing western blotting using maize SSI antibody. There was one protein in banana, two in basella, one in carrot, two or more in maize, one or two in green beans, two in rice, none in rutabaga and two in sweet potato, and two or three in wheat, were recognized by maize SSI antibody (Figure B). In order to confirm that these proteins that were bound to the glucan in the native gel and cross-reacted with maize SSI antibody posses starch synthase activity, a renaturing gel was run (see experimental procedures for details). These gels revealed both synthetic and degradative enzyme activity (Figure C). There were two proteins in banana, two in basella, one in maize, and two in wheat that possessed synthetic activity. Degradative enzyme activity was revealed in carrot, green bean, sweet potato and wheat (C). Figure D shows mobility of starch synthase enzymes of Basella alba in native gels containing no substrates (Controls).

FIG. 18A shows SDS Gel Electrophoresis;

FIG. 18B shows Western Blot Using Maize SSI antibody;

FIG. 18C shows Gel Electrophoresis to Detect Enzyme Activities; and

FIG. 18D shows Native gel Electrophoresis of Basella leaf extracts to detect SS enzyme like activity.

FIG. 19. Shows a native gel containing 0.05% potato amylopectin and displays the differences in the mobility revealed by activity staining of maize SSI, GBSS (purified from the granules) and SSIIa enzymes.

### DETAILED DESCRIPTION OF THE INVENTION

Starch is deposited in granular storage bodies in most higher plants and is composed of amylose and amylopectin. Amylose is a lightly branched glucan polymer without any specific higher order of complexity. Amylopectin is composed of glucan chains arranged in a repeating structure which is made up of a highly branched amylopectin backbone arranged with the branches primarily located in an amorphous region, followed by a highly ordered crystalline lamella region lacking in branches. This crystalline region has been represented in models as a “side chain liquid crystal”, where it's mobility state is determined by the degree of order amongst the liquid crystals. Changing the degree of order then has the effect of changing the cooking properties of the starch. In normal starches there is already known to be considerable order in these liquid crystalline lamella regions, but due to this invention this order can be increased further or even decreased. It is generally known that after degrading-away the highly branched region, the remaining glucans are found to vary in chain length quite considerably. This variation in chain length is one factor determining the degree of order in the lamella region. By increasing or reducing this variation in chain lengths, useful improvements in the properties of amylopectin inside the starch granule as well as its properties after being denatured. In another aspect of this invention is increasing the efficiency of synthesis of amylose. Thus it is possible to significantly change the properties of amylopectin and amylose with consequent changes in uses of different starches. These changes in starch properties can be characterised using various Theological instruments. A further embodiment of this invention is to increase the amount of starch formed within the developing granule as a result of a more tightly ordered array of liquid crystals. In this instance the amylopectin chains which vary in chain length are made more uniform and this has the effect in making the starch pack more densely into the same space in the liquid crystal lamellae region. This has the potential not only to change starch properties, but also to increase yield as well as to increase the density of individual starch granules. This is useful because it will increase yield and also facilitate easier isolation and purification of the new starch.

Some previously well characterized ways of altering amylose and amylopectin chain length distribution involves using mutants and/or biotechnology to alter the ratios of enzymes responsible for synthesizing starch. These enzymes include the various isoforms of starch synthases, branching enzymes and debranching enzymes. This patent envisions ways of further altering amylose and amylopectin structure by engineering changes in starch synthase proteins. In particular, specific regions of certain proteins will be linked to other regions from different proteins. This engineering is made possible by the present invention which provides the specific functions of certain domains within the starch synthase proteins. Using a combination of biochemical studies and molecular evaluation, four different regions were identified within the starch synthase class of proteins. Each domain has a different yet specific function and each function is different between the different starch synthase isoforms. First is a glucan association domain (GLASS) which is responsible for determining the chain length specificity of the enzymes and their ability to associate with starch. Second is a linker domain (LINKR) responsible for proper substrate processing and separate GLYTR and GLASS domains This domain also facilitates in setting the limits on the length of glucan chains being made. Third is a glucosyl transferase domain (GLYTR) responsible for the stepwise addition of a catalytically-activated glycosyl moiety to the non-reducing end of the amylose or amylopectin glucan chain. Fourth is the C-terminal end (CTEND),which is responsible for proper folding of the overall protein. The present invention provides, in certain embodiments, proteins, peptides and/or polypeptides which are a mix and/or match these four different domains selected from different starch synthase proteins. Since many of these proteins have been identified and cloned it is possible to envision many ways to bioengineer many different combinations of new enzymes. Such new combinations of enzymes will have significantly new properties such as increased enzyme catalytic efficiency as well as changed specificity with respect to glucan chain length. A further extension of this invention is to replace the alpha-1,4glycosyl transferase catalytic domain (GLYTR) with another glycosyl transferase domain having different properties such that the glucan addition would be in a different 3314 configuration in the amylopectin molecule. For example this enhancement could place alpha-1,3 glucans in amongst the alpha-1.4 glucans normally found in starch.

To achieve these changes in amylopectin structure and hence the properties of the starch, it is necessary to create new genes encoding these novel starch synthesizing enzymes. By bioengineering each domain from a specific target enzyme, it is possible to form a fusion protein containing each of these four domains. The domains have to placed in a specific order from N-terminus through to the C-terminal end (for example: GLASS, LINKR, GLYTR, CTEND). Next the new genes are engineered so that they are expressed in the plant. The enzymes are expressed during starch formation and have to be engineered to contain a transit peptide sequence. This will ensure correct targetting of the proteins to the amyloplast where starch is synthesised. Using biotechnological techniques well known in the art, the starch enhancement envisioned herein can be done in any organism and more particularly any organism that stores or synthesizes starch.

I. Fusion Peptides of Starch Synthases

Fusion proteins, also called “hybrid proteins” are polypeptide chains that contain of two or more proteins fused together into a single polypeptide. U.S. Pat. Nos. 5,202,247 and 5,137,819 describe hybrid proteins having polysaccharide binding domains and methods and compositions for preparation of hybrid proteins capable of binding to polysaccharide matrix. Also, U.S. Pat. No. 5,202,247 describes a hybrid protein linking a cellulase-binding region to a peptide of interest. A number of patents have outlined improvements in methods of making hybrid peptides or specific hybrid peptides targeted for specific uses. For example, U.S. Pat. No. 5,635,599 reports a circularly permuted ligand with high specificity and good binding affinity as part of the hybrid peptide. U.S. Pat. No. 5,648,244 describes a method for producing a hybrid peptide with a carrier peptide. This nucleic acid region when recognized by a restriction endonuclease creates a nonpalindromic 3-base over hang that allows the vector to be cleaved.

There are reports of vectors for engineering modification in the starch pathway of plants by use of a number of starch synthesis genes in various plants. Some of these polysaccharide enzymes bind to starch, glycogen or cellulose. The U.S. Pat. No. 5,349,123 described a vector containing DNA to form glycogen biosynthetic enzymes within plant cells to introduce changes in potato starch.

The present invention provides however fusion proteins made by combining or pairing various functional polypeptide domains of starch synthases to introduce a modification in the starch structure (Table V). In the present invention, the starch association domain of GBSS enzyme is fused with the functional or catalytic domains of other various SS enzymes with different and specific functionalities to introduce modifications to starch structure.

Preferred recombinant nucleic acid molecules of this invention comprise DNA encoding the above domains (“GLYTR” or “GLASS” Domains) from any organism and comprise gene sequences set forth in the tables hereof.

Preferred plasmids of this invention are adapted for use with specific hosts. Plasmids comprising a promoter, a plastid-targeting sequence, a nucleic acid sequence encoding the above domains and a terminator sequence are provided herein. Such plasmids are suitable for insertion of DNA sequences encoding the “GLYTR”or “GLASS”domains with a LINKR or space sequence in between for expression in selected hosts. The invention includes plasmids comprising promoters adapted for both prokaryotic and eukaryotic hosts. The said promoters may also be specifically adapted for expression in monocots or in dicots.

The said fusion polypeptide according to the present invention has five regions.

| | | | | |
|---|---|---|---|---|
| N-terminal | GLASS | LINKR | GLYTR | CTEND |
| ARM_{(transit peptide)} | Peptide | Peptide | Peptide | Peptide |

LINKR peptide is the region between the GLASS and GLYTR and can comprise any of the sequences listed in SEQ ID NO's 243-339.

CTEND is the C-terminal region of GBSS and similar proteins and can comprise 20 to 40 amino acid residues from the list provided in Seq ID NO.I

The DNA Construct for expressing the fusion protein domains within the host, broadly is as follows:

| | | Transit | | | | |
|---|---|---|---|---|---|---|
| | | Peptide/ | | | | |
| | | And/or | | | | |
| | | N-term | | | | |
| Promoter | | ARM | | | | |
| Termi- | | Coding | Coding Regions for fusion peptides | | | |
| nator | Intron* | region | GLASS | LINKR* | GLYTR | CTEND* |

*= optional components

As is known in the art, a promoter is a region of DNA controlling transcription. Different types of promoters will be selected for different hosts. Lac and T7 promoters work well in prokaryotes, the ³⁵S CaMV promoter works well in dicots. And the polyubiquitin promoter works well in many monocots. Other suitable promoters include maize 10 kDa Zein promoter, GBSS promoter, ST1 promoter, TR1 promoter, napin promoter etc. Any number of different promoters are known to the art can be used within the scope of this invention. It can be constitutive, inducible, tissue specific and may be homologous or heterologous to the said plant.

Also, as is known to the art, an intron is a nucleotide sequence in a gene that does not code for the gene product. One component of an intron that often increases expression in monocots is the Adh1 intron. This component of the construct is optional.

The transit peptide-coding region is a nucleotide sequence that encodes for the translocation of the protein into organelles such as plastids and mitochondria. It is preferred to choose a transit peptide that is recognized and compatible with the host in which the transit peptide is employed. In this invention the plastid of choice is the amyloplast. An example is Ferredoxin transit peptide that worked well for us in the past.

It is preferred that the hybrid polypeptide be located within the amyloplast in cells such as plant cells that synthesize and store starch in amyloplasts. If the host is a bacterial or other cell that does not contain an amyloplast, there need not be a transit peptide-coding region.

A terminator is a DNA sequence that terminates the transcription. The fusion polypeptides may also include post-translational modifications known to the art such as glycosylaiton, acylation, and other modifications not interfering with the desired activity of the polypeptide.

Brief Description of the Procedure for Developing Fusion Polypeptide

A genetic construct encoding a fusion of the invention may be obtained by “combining” the nucleotide sequences encoding at least one desired protein or polypeptide with at least one nucleotide sequence that codes for “GLYTR” or “GLASS” domains optionally with or via one or more sequences that encode a “LINKR” and “CTEND” sequences as described above, in such a way that expression of the combined sequences in the desired plant or any other organism leads to the formation of the fusion.

Genes can be cut and changed by ligation, mutation agents, digestion, restriction and other such procedures for example, as outlined in Sambrook et al., “Molecular Cloning: A Laboratory Manuel”, 2^{nd} edition, Vols1-3, Cold Spring Harbor Laboratory(1989).

In addition, the sequences encoding for the “GLYTR” or “GLASS” domains ,“LINKR” and “CTEND” regions can be provided synthetically using known DNA synthesis techniques or isolated from a suitable biological source.

In addition to the elements mentioned above, the genetic construct encoding the fusion proteins of the invention may further contain all other elements known per se for nucleic acid sequences or genetic constructs, such as other control elements, terminators, translation or transcription enhancers, integration factors, signal sequences, and selection markers etc., that are preferably suited for use in the transformation of the host plant. The sequences that encode these further elements of the construct may be isolated from a biological source or synthesized synthetically. The one or more nucleotide sequences encoding these elements of the construct again can be combined with the nucleotide sequence encoding the fusion in a manner described in in Sambrook et al., “Molecular Cloning: A Laboratory Manuel”, 2^{nd} edition, Vols.1-3, Cold Spring Harbor Laboratory(1989). The genetic construct encoding the fusion proteins may also include post-translational modifications known to the art such as glycosylation, acylation, and other modifications not interfering with the desired activity of the polypeptide.

Construct Development

According to one preferred embodiment, the genetic construct encoding the fusion is preferably in a form suitable for transformation of a plant, such as a vector or plasmid. The recombinant nucleic acid sequence of this invention is inserted into a convenient cloning vector or plasmid. For the present invention the preferred host is a starch granule-producing organism. However, bacterial hosts can be employed. In bacterial host, transcriptional regulatory promoters include lac, TAC, trp and the like. Additionally, DNA coding for transit peptide most likely would not be used and a secretory leader that is upstream from the structural gene may be used to get the polypeptide into the medium. Alternatively, the product is retained in the host and the host is lysed and the product isolated and purified by starch extraction methods or by binding the material to a starch like matrix such as amylose, or amylopectin, glycogen or the like to extract the product.

The cloning vector may contain coding sequences for a transit peptide to direct the plasmid into the correct location. Examples of transit peptide sequences are shown in

Coding sequences for other transit peptides can be used. Transit peptides naturally occurring in the host to be used are preferred.

Attached to the transit peptide coding sequence is the DNA sequence encoding the N-terminal end of the fusion protein domain. The direction of the sequence encoding the fusion protein is varied depending on whether sense or antisense transcription is desired. DNA constructs of this invention specifically described herein have the sequence encoding the “GLASS” domain at the N-terminus end but the “GLYTR” domain can also be at the N-terminus end and the “GLASS” sequence following. The same procedure applies to inserting “LINKR” and “CTEND” regions if needed. At the end of theDNA construct is the terminator sequence. Such sequences are well known in the art.

The cloning vector is transformed into a host. Introduction of the cloning vector, preferably a plasmid, into the host can be done by a number of transformation techniques known to the art. These techniques may vary by host but they include microparticle bombardment, micro-injection, Agrobacterium transformation, electroporation, and the like. If the host is a plant, the cells can be regenerated to form plants. Methods of regeneration of plants is known in the art. Once the host is transformed and the proteins expressed therein, the presence of the DNA encoding the fusion protein in the host is confirmed. Transcript levels can be measured and the presence of fusion proteins may b econfirmed by Western blotting or ELISA or as a result of change in the Theological properties of starch.

With regard to starch synthase fusion proteins, WO 98/14601 provides similar methods to generate naturally occuring starch that has been modified to comprise the payload peptide and not associated with bringing any structural changes to the starch or glucan chain lengths. The present invention is based, in part, on the further discoveries regarding SS enzymes and their constituent domains (detailed information provided herein below) and further evidence for the mechanism of protein entrapment in the starch granules. The present invention provides therefore methods for making and using ‘starch synthase fusion proteins’ and producing transgenic plants capable of producing “structurally modified starch” or starch granules as described herein below. Such “structurally modified starch” of the present invention differs from naturally occurring starch in the plant by at least one property thereof, such as crystallinity, branching degree, glucan composition and glucan chain length.

With regard to sequences of the starch association domain, WO 98/14601 described the idea of a hybrid polypeptide comprising: (a) a starch binding domain, and (b) payload polypeptide fused to said starch binding domain. Said starch binding domain is referred as “starch-encapsulating domain”. It may be any starch binding domain known per se, for instance derived from soluble starch synthase I, IIa, IIb, Du1, GBSS, branching enzyme I, IIa, IIb, and/or glucoamylase polypeptides. The present invention provides, in at least one embodiment, a polypeptide sequence of GBSS that will enable fusion proteins to be entrapped in the granular matrix.

With regard to structural modification of starch, WO 98/14601 provides a “peptide-modified starch” for nutritious feed. WO 98/14601 provides for encapsulation of desired amino acids or peptides within starch and specifically within starch granule to increase the plants capacity to produce a specific protein, peptide or provide an improved aminoacid balance. WO 98/14601 defined modified starch as the naturally occurring starch that has been modified to contain a payload polypeptide. Payload polypeptides are described therein as hormones or other medicaments, e.g. insulin in a starch encapsulating form to resist degradation by stomach acids for producing the payload polypeptides in easily purified form or to enhance the amino acid content of particular amino acids in the starch to provide grain feeds enriched in certain amino acids. The present invention provides, in some embodiments, methods of making and using “structure-modified starch”, such as may be used in various industrial applications.

WO 98/14601, provides for a payload polypeptide which is not endogenous to the starch encapsulating region whose expression is desired in association with this region to express a starch containing the payload polypeptide. Specific examples of payload polypeptides described therein are hormones, eg. Insulin, a growth factor like somatotropin, calcitonin, beta endorphin, urogastrone, beta globin, myoglobin, human growth hormone, angiotensin, proline, proteases, beta-galoctosidase, and cellulase, antibody, an enzyme, immunoglobulin, or dye, prolactin, and serum albumins etc.

The present invention provides polypeptides, in at least one embodiment, which are capable of interacting with starch or starch granules and show an affinity and/or enzymatic activity with starch, such that the polypeptides of the present invention may be associated with modifying glucan chain lengths of amylopectin. The present invention further provides for fusion proteins containing one starch association domain and one catalytic domain of SS enzyme that alters, converts and modifies starch structure. The present invention provides a means and methods therefore to modifying starch structure.

II. Domains of the Enzymes Involved in Starch Metabolism and their Potential Uses

Enzymes, particularly from microorganisms, are known that interact with starch. These enzymes generally contain one or more catalytic domain, and one or more regions that can bind to starch or starch granules and referred to as “starch binding domains” or “starch binding regions”. Starch association-domains for starch synthesis enzymes in higher plants however are not known or described in the literature.

Svensson et al., Biochem. J. (1989), 264, 309-311, described the sequence homology between putative starch binding domains from α-amylase from Streptomyces limosus, β-amylase from Clostridium thermosulfurogenes, glucoamylase from A.niger, maltogenic α-amylase from Bacillus stearothermophilus, malto-tetraose forming amylase from Pseudomonas slutzeri, CGTase from Bacillus, CGTase from Klebsiella pneumoniae and glucoamylase from Rhizopus oryzae. Various starch-binding domains were also compared by Janecek& Sivcek, 1999 (FEBS letters 456, 119-125). It has been suggested that some conserved tryptophan residues and the amino acids directly adjacent may play an important role in starch binding (vide Goto et al., Appl. Environ.Microbiol.,1994, p3926-3930, Chen et al., Protein engineering, 1995, Vol 8:1049-1055, Williamson et al., Biochemistry, 1997, 36:7535-7539). Chen et al., 1991, Gene 99, 121-126, Biotechnol. Prog. 1991, 7: 225-229 described a fusion of B3-galactosidase and the starch-binding domain from an Aspergillus glucoamylase, plasmids encoding such a fusion, and expression in E. coli. The starch-binding region is used to increase the affinity of β-galactosidase for starch granules, in particular as an affinity tail for recovery or enzymatic immobilization using native starch granules as an absorbant.

The use of starch binding domain fusions in oral care compositions that contain such fusions is described in the patent WO 98/16190. The fusions were prepared by expression of an appropriate expression vector in a suitable microorganism. WO 99/15636 describes starch-binding domains, and in particular the “D” and “E-domains” of the maltogenic amylase from Bacillus StearothermophilusC599, and expression thereof in a Bacillus host cell. This patent also described fusions of starch binding domain and a reporter gene such as GFP to monitor the expression of the starch binding domains in the Bacillus host. This patent only describes expression in Bacillus host and does not describe fusion of starch binding domain and an enzyme that can interact with starch or starch granule.

Dalmia et al., Biotechnology and Bioengineering, 1995, 47:575-584 described fusions of β-galoctosidase and the starch binding domains of glucoamylase I of Aspergillus awamori and of cyclodextrin glucanotransferase (domain E of CGTase) from Bacillus macerans, respectively, plasmids encoding such fusions, and expression of said fusions in E. coli. The fusion proteins thus obtained are said to bind specifically to potato starch, corn-starch, and cross-linked amylose. As a possible application, the use of the starch binding domains as an “affinity tag” is suggested. Similarly, Dalmia et al., 1994, Enzyme Microb.Technol, vol 1. describe fusions containing a starch binding domain from Aspergillus niger glucoamylase, which is again used as an affinity tail to facilitate the one step purification of the target β-galactosidase. The use of cellulose binding domains as an affinity tag for protein purification (i.e. a fusion of a cellulose binding domain from cellulase and a-galactosidase) has also been described in the art by vide Ong et al., 1989, Trends in Biotechnology, 7:239-243.

All the above references describe fusions of a starch binding domain and an enzyme, said fusions are only expressed in micro-organisms such as E. coli. The starch binding domain is included only as a “tail” or tag in order to facilitate the isolation and purification of the desired enzyme activity from the bacterial culture medium.

The use of fusion proteins in plants in situ, in particular entrapped in the starch granules has not been previously described in the literature. WO 98/14601 describes entrappment of a “payload polypeptide” in order to make a nutritionally enriched starch. The expression in plants of fusions containing enzyme doamins that can alter the length of glucan chains in amylopectin, and there by produce modified starch had not been described or suggested however in the literature.

The present application provides a means of altering starch structure and deposition in plants by using novel starch synthesizing enzymes whose catalytic properties have been found to be substantially different from known enzymes. Starches produced in plants expressing these enzymes, which are also provided by the present invention, are substantially new and novel.

The genetic constructs described in this patent may be of plant, fungal, bacterial or animal origin, and are generally incorporated into the plant genome by sexual crossing or by transformation. The enzyme gene products may be an additional copy of a wild-type gene or may encode a modified enzyme with improved properties. Incorporation of the enzyme gene construct(s) into crop plants may have varying effects depending on the amount and type of enzyme gene(s) introduced. It may also increase the plant's capacity to produce starch, in particular by altering the temperature optimum for enzyme activity, giving increased yield. It may also result in production of starch with an altered fine structure (or quality) as the exact structure depends on the novel enzyme introduced. In examples where starch structure has been altered there have generally been starch-synthesizing enzymes expressed in a wild-type background via sexual crossing. The following patent applications describe this concept in detail: PCT/GB92/01881; US application numbers 4,35,020 and 9,30,935, European publication number EPA 3,68,506 (published May 16, 1990); UK patent application number 9,218,185.8. The disclosures of these applications are hereby incorporated by reference.

III. Starch Synthases

Both prokaryotic and eukaryotic cells use polysaccharides as a storage reserve. In the prokaryotic cell the primary reserve polysaccharide is glycogen. Although glycogen is similar to the starch found in most vascular plants it exhibits different chain lengths and degrees of polymerization. In many plants, starch is used as the primary reserve polysaccharide. Starch is stored in the various tissues of the starch bearing plant. Starch is made of two components in most instances; one is amylose and the other amylopectin. Amylose is formed as essentially linear glucans and amylopectin is formed as a more highly-branched chains of glucans. Typical starch has a ratio of 25% amylose to 75% amylopectin. Starch synthases (EC 2.4.1.11) elongate starch molecules and act on both amylose and amylopectin. Starch synthase (SS) activity can be found associated both with the granule and in the stroma of the plastid. Variations in the amylose to amylopectin ratio in a plant can affect the properties of the starch. Additionally starches from different plants often have different properties. Maize starch and potato starch appear to differ due to the presence or absence of phosphate groups. Certain plants' starch properties differ because of mutations that have been introduced into the plant genome. Mutant starches are well known in maize, rice, and peas and the like.

The changes in starch branching or in the ratios of the starch components result in different starch characteristic. One characteristic of starch is the formation of starch granules that are formed particularly in leaves, roots, tubers and seeds. These granules are formed during the starch synthesis process. Certain synthases of starch, particularly granule-bound starch synthase, soluble starch synthases and branching enzymes are proteins that are “granule bound” within the starch granule when it is formed (Smith et al., 1997, Ann. Rev. Plant Physiol.Plant Mol. Biol. 48, 67-87).

Different isoforms of soluble starch synthase have been identified and cloned in pea (Denyer and Smith, 1992, Planta 186: 609-617; Dry et al., 1992, Plant Journal, 2: 193-202), potato (Edwards et al., 1995, Plant Physiol 112: 89-97; Marshall et al., 1996, Plant Cell 8: 1121-1135), wheat (Gao and Chibbar, 2000; Genome. Vol 43: 768-775), and in rice (Baba et al., 1993, Plant Physiol. 103: 565-573), while barley appears to contain multiple isoforms, some of which are associated with starch branching enzyme (Tyynela and Schulman, 1994, Physiol. Plantarum 89: 835-841).

The capacity for starch association of the bound starch synthase enzyme is well known. Various enzymes involved in starch biosynthesis are now known to have differing propensities for binding as described by Mu-Forster et al. (1996, Plant Phys. 111: 821-829). Granule-bound starch synthase (GBSS) activity is strongly correlated with the product of the waxy gene (Shure et al., 1983, Cell 35: 225-233). The synthesis of amylose in a number of species such as maize, rice and potato has been shown to depend on the expression of this gene (Tsai, 1974, Biochem Gen 11: 83-96; Hovenkamp-Hermelink et al., 1987, Theor. Appl. Gen. 75: 217-221). Visser et al. described the molecular cloning and partial characterization of the gene for granule-bound starch synthase from potato (1989, Plant Sci. 64(2):185-192).

In starch producing plants starch is usually synthesized in the form of starch granules. A number of enzymes in the plant especially the ones involved in the starch synthesis and degradation interact in vivo with these granules. These include the enzymes such as starch synthases, branching enzymes and debranching enzymes, and amylases etc. for which reference is made to Mu, C., Ham, C., Ko, Y. T., Singletary, G. W., Keeling, P. L. and Wasserman, B. P. Plant J., 1994, 6:151-159, Smith, A. M., Denyer, K., and Martin, C. Annu.Rev.Plant Physiol. Mol. Biol., 1997, 48:67-87 and Martin C., and Smith A.M. The Plant Cell, 1995, 7:971-985. However, compared to any other enzyme, granule bound starch synthase (GBSS) is the most abundant protein entrapped in the starch granules with highest affinity to amylopectin.

The present invention also classifies maize α-1,4 glucan transfereases or starch synthases based on their specificities to process various lengths of α-1,4 glucan chains in the amylopectin cluster. For example, according to the present invention, SS enzymes are defined in 4 classes. ‘Class I’ enzymes that include maize SSI and like enzymes, and preferentially elongate α-1,4 glucan chains to synthesize shorter A and B1 chains; ‘Class II’ enzymes that include maize SSIIa and SSIIb and like enzymes, and preferentially add a glucose unit(s) to α-1,4 glucan chains to synthesize longer A and B1 chains and intermediate B2 or B3 chains; ‘Class III’ enzymes that include maize SSIII and preferentially add a glucose unit(s) to α-1,4 glucan chains to synthesize longer A, B1, B2 and B3 chains as well as longer B3 or C chains of amylopectin. “Class IV” enzymes include GBSS and preferentially add a glucose unit(s) to α-1,4 glucan chains to synthesize longer B3 or C chains of amylopectin as well as amylose. In maize or any other crop when transformed to express or overexpress any one specific class of starch synthases described above will result in an increased number of glucan chains in that specific class. This patent application relates to modification of starch structure by introduction/entrapment of polypeptide domains of other soluble starch synthases (SSS) in addition to GBSS (in the form of GBSS+SSS enzyme fusion proteins) within the starch granule matrix. Therefore, the present invention provides new starch synthases other than GBSS or SSI within the starch granule matrix. These enzymes contain starch association domain of either GBSS or SSI as described above and herein which provides starch association properties similar to wild type GBSS or SSI while retaining the α-1,4 glucan transferase activity (catalytic activity) of either GBSS or soluble starch synthases such as GBSS, SSI, SSIIa, SSIIb, and SSIII and the like. Starches produced in plants expressing these enzymes are substantially new and novel.

The use of cDNA clones of animal and bacterial glycogen synthases are described in PCT/GB92/01881. The use of cDNA clones of plant soluble starch synthases has been reported. For example, the amino acid sequences of pea soluble starch synthase isoforms I, and II were published byDry et al. (1992, Plant Journal, 2:193-202) and SSIII (Gao et al., 1998). The amino acid sequence for rice soluble starch synthase was described by Baba et al.,(1993, Plant Physiology). This last sequence (rice SS) incorrectly cites the N-terminal sequence and hence is misleading. Presumably this is because of some extraction error involving a protease-degradation or other inherent instability in the extracted enzyme. The correct N-terminal sequence (starting with AELSR) is present in what they refer to as the transit peptide sequence of the rice SS.

Branching enzyme [α1,4Dglucan: α 1,4Dglucan 6D(α1,4Dglucano) transferase (E.C. 2.4.1.18)], some times called Q-enzyme, converts amylose to amylopectin. A segment of a α 1,4Dglucan chain is transferred to a primary hydroxyl group in a similar glucan chain. Bacterial branching enzyme genes and plant sequences have been reported (rice endosperm: Nakamura et al., 1992, Physiologia Plantarum, 84:329-335 and Nakamura and Yamanouchi, 1992, Plant Physiol., 99:1265-1266; pea: Smith, 1988, Planta, 175:270-279 and Bhattacharyya et al., 1990, J. Cell Biochem., Suppl. 13D:331; maize endosperm: Singh and Preiss, 1985, Plant Physiology, 79:34-40; VosScherperkeuter et al., 1989, Plant Physiology, 90:75-84; potato: Kossmann et al., 1991, Mol. Gen. Genet., 230(12):39-44; cassaya: Salehuzzaman and Visser, 1992, Plant Mol Biol, 20:809-819). The sequence of maize branching enzyme I was investigated by Baba et al., 1991, BBRC, 181:87-94. Starch branching enzyme II from maize endosperm was investigated by Fisher et al.(1993, Plant Physiol., 102:1045-1046). The use of cDNA clones of plant, bacterial and animal branching enzymes have been reported. The nucleotide and amino acid sequences for bacterial branching enzymes (BE) are known from the literature. For example, Kiel et al. cloned the branching enzyme gene glgB from Cyanobacterium synechococcussp PCC7942 (1989, Gene (Amst), 78(1): 918) and glycogen branching enzyme gene (glgB) from Bacillus stearothermophilus and expressed in Escherichia coli and Bacillus subtilis Kiel J A. Boels J M. Beldman G. Venema G. (1991, Molecular & General Genetics. 230(1-2):136-44). The genes glc3 and gha1 of S. cerevisiae are allelic and encode the glycogen branching enzyme (Rowen et al., 1992, Mol. Cell Biol., 12(1): 22-29). Matsumomoto et al. investigated glycogen branching enzyme from Neurospora crassa (1990, J. Biochem., 107:118-122). The GenBank/EMBL database also contains sequences for the E. coli glgB gene encoding branching enzyme.

A common characteristic of SS clones is the presence of a KXGGLGDV consensus sequence that is believed to be the ADP-Glc binding site of the enzyme (Furukawa et al., 1990, J Biol Chem 265: 2086-2090; Furukawa et al., 1993, J. Biol. Chem. 268: 23837-23842). See below for example, the SS enzymes from various organisms

Granule Bound Starch Synthases (GBSS)

Accession Numbers:

gi|2833387|sp|Q43654|;gi|2833377|sp|;gi|2833381|sp|Q42857|; gi|2833388|sp|Q43784|gi|2829792|sp|P93568|; gi|2833383|sp|Q43092|; gi|136757|sp|P04713|; gi|136755|sp|P09842|; gi|267196|sp|Q00775|gi|2833382|sp|Q42968|; gi|2833384|sp|Q43093|; gi|6136121|sp|082627|; gi|2833385|sp|Q43134|;gi|136765|sp|P27736|; gi|2833390|sp|Q438471; gi|136758|sp|P19395|; gi|2833389|sp|Q43846|;gi|2842612|sp|Q59001|; gi|2811062|sp|O08328|; gi|729578|sp|P39125|; gi|2829618|sp|P74521|; gi|1169908|sp|P08323|; gi|121295|sp|P05416|; gi|729577|sp|P39670|; gi|1169909|sp|P45179|;

Soluble Starch Synthases (SSS)

Accession Numbers

gi|2129898|pir||S61505;gi|7489826|pir||T01265;gi|9502143|gb|AAF87999.1|AF258608_{—}1 gi|2833390|sp|Q43847|;gi|7489274|pir||T07663;gi|4582789|emb|CAB40374.1|;gi|8573760|g b|AAC17969.2|;gi|8953573|emb|CAB96627.1|;gi|2833384|sp|Q43093 |;gi|12019656|gb|AAD 45815.2|gi|2833387|sp|Q43654|;gi|6467503|gb|AAF13168.1|AF173900_{—}1;gi|7433871 |pir||S7 4473gi|7188796|gb|AAF37876.1|AF234163_{—}1;gi|8708896|gb|AAC17970.2|;gi|8953571 |emb |CAB96626.1|;gi|2833389|sp|Q43846|;gi|10177090|dbj|BAB10396.1|;gi|9502145|gb|AAF88 000.1|;gi|7489695|pir||T06798;gi|5825480|gb|AAD53263.1|AF155217;gi|9369336|emb|CAB 99210.1|gi|8901183|gb|AAC17971.2|;gi|5880466|gb|AAD54661.1|;gi|2829792|sp|P93568|; gi|7488349|pir||T04926;gi|3192881|gb|AAC19119.1|;gi|7529653|emb|CAB86618.1|;gi|6103 327|gb|AAF03557.1|;gi|7489712|pir||T01414;gi|7489711|pir||T01209;gi|9369334|emb|CAB9 9209.1|gi|5295947|dbj|BAA81848.1|; gi|549232|dbj|BAA07396.1|;gi|7489710|pir||T01208;gi|2833377|sp|Q40739|; gi|729578|sp|P39125|; gi|3688125|emb|CAA06959.1|;gi|9587348|gb|AAF89274.1|; gi|9587352|gb|AAF89276.1|AF286003_{—}1|;gi|9587319|gb|AAF89261.1|AF285986_{—}1|;gi|9587 329|gb|AAF89266.1|AF285991_{—}1;gi|9587337|gb|AAF89270.1|AF285995_{—}1;gi|9587313|gb|AAF89258.1|AF285983_{—}1|gi|9587317|gb|AAF89260.1|AF285985_{—}1;gi|9587311|gb|AAF892 57.1|AF285982_{—}1

gi|9587295|gb|AAF89249.1|AF285974_{—}1;gi|9587307|gb|AAF89255.1|AF285980_{—}1;gi|9587 321|gb|AAF89262.1|AF285987_{—}1;gi|9587297|gb|AAF89250.1|AF285975_{—}1;gi|958730|gb|AAF89252.1|AF285977;1,gi|9587339|gb|AAF89271.1|AF285996_{—}1;gi|958733|gb|AAF892 67.1|AF285992_{—}1

gi|9587305|gb|AAF89254.1|AF285979_{—}1; gi|9587335|gb|AAF89269.1|AF285994_{—}1; gi|958 7341|b|AAF89272.1|AF285997_{—}1; |gi|9587343|gb|AAF89273.1|AF285998_{—}1; gi|9587325|g b|AAF89264.1|AF285989_{—}1; gi|9587293|gb|AAF89248.1|AF285973_{—}1; gi|95873231 |gb|AAF 89263.1|AF285988_{—}1gi|9587333|gb|AAF89268.1|AF285993_{—}1; gi|9587299|gb|AAF89251.1|AF285976_{—}1; gi|9587327|gb|AAF89265.1AF285990_{—}1; gi|9587309|gb|AAF89256.1|AF28 5981_{—}1; gi|9587303|gb|AAF89253.1|AF285978_{—}1

Hybrid proteins or fusion proteins are polypeptide or peptide chains that contain two or more proteins or peptides fused together into a single polypeptide or peptide. Any of the starch synthase protein domains from the above listed or unlisted may be recombined as an embodiment of the present invention so as to control the interaction between SS and its substrates amylose or amylopectin. Such a recombination will allow to control the glucan chain lengths synthesized in the starch granule and therefore, control the useful properties of the starch.

IV. Glucan Association and Chain Length Specificity Characteristics of SS Enzymes:

Glucan-affinity gel electrophoresisin was used, and is described herein, as a tool to discover the precision and mechanism of interaction between the starch synthase enzymes, SSI, and GBSS, and their glucan-substrates, with which the glucan chain-lengths are determined by various starch synthases. SSI was found to have a greatly elevated affinity for increasing chain lengths of α-1,4 glucans (FIG. 8, A, B, C). Contrarily, the activity of SSI enzyme was decreased with increase in the avg. OCL of a-1, 4 glucans (FIG. 8D). Deletion of the N-terminal arm of SSI protein did not affect any of the glucan-binding characteristics (FIG. 9). Moreover, SSI enzyme activity is proportional to the average outer chain lengths of a given glucan molecule, which explains why SSI enzyme rate of catalysis is higher using glycogen (with shorter outer chains of dp ˜6.5) than with starch (avg. OCL ˜14.5) or amylopectin (avg. OCL˜11.5) (Imparl-Radosevich et al., 1998a; and references there in). During enzyme catalysis using SSI, majority of the 14C-label was incorporated into glucan chains with average dp less than 10 (FIGS. 10A & B). In contrast, GBSS displayed least affinity to glycogen (Table 1), however, with increasing outer chain lengths of glycogen, GBSS was found to have both an elevated affinity (FIG. 11) and catalytic activity (FIG. 11). The enzyme had the highest affinity for amylopectin and it preferred longer chains (>dp 20) of this molecule for chain extension as well (FIG. 11). In order to validate these results using SSI or GBSS, results to SSIIa, which does not entrap in the starch during the synthesis of starch granule, were compared. Unlike GBSS or SSI enzymes, SSIIa did not have any preference either for binding or for catalytic activity (FIGS. 12 and 13). SSIIa did not have any increased glucan binding with increase in the outer chain lengths. The activity of SSIa and Du 1 did not increase or decrease by altering the average outer chain lengths of glucan (FIG. 13). SSIIb did not prefer longer chained glycogen, but unlike SSI, the activity did not sharply drop after average outer chain length of dp 9. However, this drop occurred at average outer dp of 14 (FIG. 13). These contrasting results among the different enzymes that are exemplified herein indicate that the starch synthase enzymes ability to associate with the carbohydrate chains enable the enzyme to be entrapped inside the starch granule. Moreover, each enzyme has it's own specificity for length of the glucan chains. These findings provide a basis for explaining why GBSS and SSI are more strongly associated with the starch granules whereas SSIIa and SSIIb are poorly or not associated with the granule.

V. GLucan ASSociation Domain (GLASS Domain) of GBSS

Using GBSS enzyme, it has been demonstrated that the glucan-binding domain is not discrete at N- or C-terminus, but may be located close to the amino acid number 103 of the protein. The smallest peptide that has affinity for glucan was found to be about 18 kDa. The C-terminal region of GBSS (˜20 amino acids long) proteins from a wide range of species is conserved, and is hydrophilic and carries a net negative charge. This C-terminal extension is absent from other starch synthase isoforms and bacterial glycogen synthases. Edwards et al. (1999) have shown that this C-terminal region of the enzyme in potato confers most of the specific properties of this isoform except its processive elongation of glucan chains. This C-terminus of maize GBSS has been shown herein not be involved in binding of the enzyme to amylopectin molecule. Sequence comparison of starch synthases from different plant species reveal that in the region of the protein following the amino acid residue number 103 is highly conserved especially for GBSS enzymes as compared to soluble starch synthases. Within this 18 kDa starch associating domain, amino acid sequence “PV(L)AGT” starting at residue number 107 is highly conserved among pea, potato and in maize GBSS. There is also another sequence starting at residue number 155, “NDWHT” which is highly conserved among all known SS enzymes in maize. Therefore, one or more of these conserved regions may confer glucan-binding properties to GBSS enzyme. Overall, it is clear that the starch-affinity domain of SS enzymes is structurally different from the starch-binding domain of the degradative enzymes and unlike these enzymes, is not a single discrete domain.

The present invention provides a glucan or starch association domain of a starch synthase, such as a granule bound starch synthase peptide or soluble starch synthase which is, in one embodiment, about 18 kDa molecular weight under reducing conditions. The starch association domain of the present invention is preferably a peptide or polypeptide fragment of granule bound starch synthase (GBSS), which has an N-terminal end which is within, at most, 50 amino acids of the amino acid corresponding to about amino acid 103 of maize GBSS enzyme. Preferably, the starch association domain of the present invention has an N-terminal end which is within, at most, 50 amino acids of the amino acid corresponding to amino acid 103 of maize GBSS and extends, at most, approximately a further 200 amino acids along toward the C-terminus of GBSS enzyme. Alternatively, the association domain of the present invention has an N-terminus as described above and a C-terminus which is within, at most, 52 amino acids of the amino acid corresponding to amino acid 148 of maize GBSS. Alternatively, the association domain of the present invention is a peptide or polypeptide of GBSS corresponding to an amino acid sequence spanning amino acid positions 103±50 amino acids to about amino acid position 251±50 amino acids of the maize GBSS enzyme. Alternatively the N- and C-termini of the association domain of the present invention may correspond to amino acid positions corresponding to amino acid positions which are, independently, plus or minus 40 amino acids from the amino acids corresponding to the amino acid positions 103 and 251, respectively, of maize GBSS enzyme; alternatively, the N- and C-termini of the association domain of the present invention may correspond to amino acid positions corresponding to amino acid positions which are, independently, plus or minus 30 amino acids from the amino acids corresponding to amino acid positions 103 and 251, respectively, of maize GBSS enzyme; alternatively, the N- and C-termini of the association domain of the present invention may correspond to amino acid positions corresponding to amino acid positions which are, independently, plus or minus 20 amino acids from the amino acids corresponding to amino acid positions 103 and 251, respectively, of maize GBSS enzyme; alternatively, the N- and C-termini of the association domain of the present invention may correspond to amino acid positions corresponding to amino acid positions which are, independently, plus or minus 10 amino acids from the amino acids corresponding to amino acid positions 103 and 251, respectively, of maize GBSS enzyme; alternatively, the N- and C-termini of the association domain of the present invention may correspond to amino acid positions corresponding to amino acid positions which are, independently, plus or minus 5 amino acids from the amino acids corresponding to amino acid positions 103 and 251, respectively, of maize GBSS enzyme; alternatively, the N- and C-termini of the association domain of the present invention may correspond to amino acid positions corresponding to amino acid positions which are, independently, plus or minus 4 amino acids from the amino acids corresponding to amino acid positions 103 and 251, respectively, of maize GBSS enzyme; alternatively, the N- and C-termini of the association domain of the present invention may correspond to amino acid positions corresponding to an amino acid position which are, independently, plus or minus 3 amino acids from the amino acids corresponding to amino acid positions 103 and 251, respectfully, of maize GBSS enzyme; alternatively, the N- and C-termini of the association domain of the present invention may correspond to amino acid positions corresponding to amino acid positions which are, independently, plus or minus 2 amino acids from the amino acids corresponding to amino acid positions 103 and 251, respectively, of maize GBSS enzyme; alternatively, the N- and C-termini of the association domain of the present invention may correspond to amino acid positions corresponding to amino acid positions which are, independently, plus or minus 1 amino acids from the amino acids corresponding to amino acid positions 103 and 251, respectively, of maize GBSS enzyme.

VI. Determination of the GLYcosyl TRansferase Domain (GLYTR Domain) of SS Enzymes

Using glucan affinity gel electrophoresis, and using SSI enzyme, the other smallest peptide that has affinity for glucan was found to be about 21 kDa. Within this 21 kDa starch associating domain, and with amino acid sequence starting the residue number 387 and with the following sequence LGLPIRPDVPLIGFIGRLD is highly conserved among all the starch synthases, and especially SSI, SSIIa, SSIIb and GBSS enzymes in maize. And, this region within or very close to the glycosyl transferase group I domain. Hence, it is highly likely that this region is involved in interaction with the glucan during the process of starch synthesis and glucan chain elongation. This is likely to be true for other SS enzymes as well due to their high sequence homology in this region. This observation also indicates that the glucosyl transferase function of SS enzymes invoves association or binding with the glucan polymer.

Therefore, the present invention provides a glycosyl transferase domain (Domain B) of a starch synthase that has affinity to glucan polymer, such as a soluble starch synthase I domain that is, in one embodiment, about 21 kDa molecular weight under reducing conditions. The glycosyl transferase domain (Domain B) of the present invention is preferably a peptide or polypeptide fragment of any starch synthase (SS), which has an N-terminal end which is within, at most, 50 amino acids of the amino acid corresponding to about amino acid 380 of maize SSI, SSIIa, SSIIb and GBSS enzymes and amino acid 1470 of maize SSIII or Du1 enzyme. Preferably, the glycosyl transferase domain of the present invention has an N-terminal end which is within 380 amino acids of maize SSI, SSIIa, SSIIb and GBSS enzymes and 1470 aminoacids of maize SSIII or Du1 enzyme, at most, 50 amino acids of the and extends, at most, approximately a further 200 amino acids along toward the C-terminus of each one of these enzymes. Alternatively, the association domain (Domain B) of the present invention has an N-terminus as described above and a C-terminus which is within, at most, 52 amino acids of the amino acid corresponding to amino acid 380 of maize SSI, SSIIa, SSIIb, and GBSS and amino acid 1470 of maize SSIII or Du1 enzyme. Alternatively, the glycosyl transferse domain (Domain B) of the present invention is a peptide or polypeptide of either SSI, SSIIa, SSIIb or GBSS corresponding to an amino acid sequence spanning amino acid positions 380±50 amino acids to about amino acid position 580±50 amino acids of the maize SS enzymes. Alternatively the N- and C-termini of the Glycosyl transferase domain of the present invention may correspond to amino acid positions corresponding to amino acid positions which are, independently, plus or minus 40 amino acids from the amino acids corresponding to the amino acid positions 380 and 580, respectively, of maize SSI, SSIIa, SSIIb, and GBSS enzyme; alternatively, the N- and C-termini of the association domain of the present invention may correspond to amino acid positions corresponding to amino acid positions which are, independently, plus or minus 30 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to amino acid positions which are, independently, plus or minus 20 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to amino acid positions which are, independently, plus or minus 10 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to amino acid positions which are, independently, plus or minus 5 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to amino acid positions which are, independently, plus or minus 4 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to an amino acid position which are, independently, plus or minus 3 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1);alternatively, the N- and C-termini of the association domain of the present invention may correspond to amino acid positions corresponding to amino acid positions which are, independently, plus or minus 2 amino acids from the amino acids corresponding to amino acid positions 380 and 580, respectively, of maize SSI, SSIIa, SSIIb and/or GBSS enzyme; and amino acid position 1470 and 1670, respectively of maize SSIII (Du-1); alternatively, the N- and C-termini of the association domain of the present invention may correspond to amino acid positions corresponding to amino acid positions which are, independently, plus or minus 1 amino acids from the amino acids corresponding to amino acid positions 378 and 545 for GBSS, 441 and 570 for SSI, 540-687 for SSIIa, 506 to 646 for SSIIb, and 1478 to 1600 for Du1 respectively.

The present invention preferably provides an isolated and/or purified domains, as described herein.

The above said “GLASS” and “GLYTR” domains of the present invention are alternatively defined as peptide or polypeptide amino acid sequences which are at least 80% identical or homologous with the above-described “GLASS” and “GLYTR”domains. Alternatively, the association domain of the present invention is more than 85% identical or homologous, or more than 90% identical or homologous or more than 95% identical or homologous, or more than 98% identical or homologous, or more than 99% identical or homologous, as compared with the above-described “GLASS” and “GLYTR”domains. One of ordinary skill in the art will readily be able to determine identical or homologous sequences by, for example, aligning sequences in question with the above-described sequence and calculating the percentage of amino acids which are different over the length of the above-described association domain. The identical or homologous peptide or polypeptide amino acid sequences of the present invention may also be identified, for example, by BLAST or Gapped BLAST search and/or comparisons, such as a comparison described or obtained by software obtainable from the NCBI website, such as through http://www.nih.gov, or http://www.ncbi.nlm.gov:80/BLAST/, or related site, or as described by Altschul, Stephen F. et al, 1997 “Gapped BLAST and PSI-BLAST: A new generation of protein data base search programs” Nucleic Acids Res. 25:3389-3402.

The above said “GLASS” and “GLYTR”domains of the present invention may also include conservative amino acid substitutions of the above-described association domain peptide or polypeptide. Such conservative amino acid substitutions will be recognized by one of ordinary skill in the art to include any of the following:

| Amino acids | Synonymous groups |
|---|---|
| Ser (S) | Ser, Thr, Gly, Asn |
| Arg (R) | Arg, His, Lys, Glu, Gln |
| Leu (L) | Leu, Ile, Met, Phe, Val, Tyr |
| Pro (P) | Pro, Ala, Thr, Gly |
| Thr (T) | Thr, Pro, Ser, Ala, Gly; His, Gln |
| Ala (A) | Ala, Pro, Gly, Thr |
| Val (V) | Val, Met, Ile, Tyr, Phe, Leu, Val |
| Gly (G) | Gly, Ala, Thr, Pro, Ser |
| Ile (I) | Ile, Met, Leu, Phe, Val, Ile, Tyr |
| Phe (F) | Phe, Met, Tyr, Ile, Leu, Trp, Val |
| Tyr (Y) | Tyr, Phe, Trp, Met, Ile, Val, Leu |
| Cys (C) | Cys, Ser, Thr, Met |
| His (H) | His, Gln, Arg, Lys, Glu, Thr |
| Gln (Q) | Gln, Glu, His, Lys, Asn, Thr, Arg |
| Asn (N) | Asn, Asp, Ser, Gln |
| Lys (K) | Lys, Arg, Glu, Gln, His |
| Asp (D) | Asp, Asn, Glu, Gln |
| Glu (E) | Glu, Gln, Asp, Lys, Asn, His, Arg |
| Met (M) | Met, Ile, Leu, Phe, Val |

Alternatively, such conservative amino acid substitutions may be any of those shown in the following:

Amino acids

| | |
|---|---|
| Ser (S) | Ser, Thr, Gln, Asn |
| Arg (R) | Arg, His, Lys |
| Leu (L) | Leu, Ile, Met, Phe, Val, Tyr, Ala, Trp |
| Pro (P) | Pro, Ala, Thr, Gly |
| Thr (T) | Thr, Ser, Ala, Trp, Gln |
| Ala (A) | Ala, Met, Ile, Leu, Phe, Val, Tyr, Trp |
| Val (V) | Val, Met, Ile, Tyr, Phe, Leu, Val, Ala |
| Gly (G) | Gly, Ala, Thr, Pro, Ser |
| Ile (I) | Ile, Met, Leu, Phe, Val, Ala, Tyr, Trp |
| Phe (F) | Phe, Met, Tyr, Ile, Leu, Trp, Val, Ala |
| Tyr (Y) | Tyr, Phe, Trp, Met, Ile, Val, Leu, Ala |
| Cys (C) | Cys, Ser, Thr, Met |
| His (H) | His, Arg, Lys |
| Gln (Q) | Gln, Gln, Asn, Thr, Ser |
| Asn (N) | Asn, Ser, Gln, Thr |
| Lys (K) | Lys, Arg, His |
| Asp (D) | Asp, Glu |
| Glu (E) | Glu, Asp |
| Met (M) | Met, Ile, Leu, Phe, Val, Ala, Tyr, Trp |
| Trp (W) | Trp, Met, Ile, Leu, Phe, Val, Ala, Tyr |

The above said “GLASS” and “GLYTR”domain polypeptide or peptide of the present invention may be a soluble starch synthase, or granule bound starch synthase, branching enzyme, and any debranching enzyme from any cereal, such as maize, wheat, rice, sorghum or barley; a fruit-producing species such as banana, apple, tomato or pear; a root crop such as cassaya, potato, yam or turnip; an oil seed crop such as rapeseed, sunflower, oil palm, coconut, linseed or groundnut; a meal crop, such as soya, bean or pea; or any other suitable species.

The above said “GLASS” domain peptide or polypeptides of the present invention include a soluble starch synthase or GBSS of any of the above cereal, fruit-producing species, root crop, oil seed crop or meal crop, for example, or fragment thereof which preferably has an N-terminus corresponding to about amino acid 103± at most 50 amino acids of maize GBSS enzyme; more preferably corresponding to amino acid 103± at most 50 amino acids of maize GBSS enzyme, and extending, at most, approximately a further 200 amino acids along toward the C-terminus of the GBSS enzyme. In this embodiment, the glucan association domain peptide or polypeptide of the present invention may extend between any amino acid position corresponding to amino acids in the range of 53-153 of maize GBSS to any amino acid position corresponding to amino acids in the range of 98-198 of maize GBSS.

The above said “GLYTR” domain peptide or polypeptides of the present invention include a soluble starch synthase or GBSS of any of the above cereal, fruit-producing species, root crop, oil seed crop or meal crop, for example, or fragment thereof which preferably has an N-terminus corresponding to about amino acid 378±at most 50 amino acids of maize GBSS enzyme; 441±at most 50 amino acids of maize SSI enzyme; 540±at most 50 amino acids of maize SSIIa enzyme; 506+at most 50 amino acids of maize SSIIb enzyme; and 1478±at most 50 amino acids of maize Du1 enzyme and extending, at most, approximately a further 200±at most 50 amino acids along toward the C-terminus of the GBSS enzyme. In this embodiment, the glucan association domain or Domain “GLASS” peptide or polypeptide of the present invention may extend between any amino acid position corresponding to amino acids in the range of 53-153 of maize GBSS to any amino acid position corresponding to amino acids in the range of 98-198 of maize GBSS.

The present invention further provides a polypeptide or peptide as described above which is more than 85% identical or homologous, or more than 90% identical or homologous or more than 95% identical or homologous, or more than 98% identical or homologous, or 99% identical or homologous, as compared with the above-described association domain peptides or polypeptides, as described above.

The present invention further provides a glucan association domain peptide or polypeptide containing the following amino acid sequence:

SEQ. ID. No. 1

“KIYGPVAGTDYRDNQLRFSLLCQAALEAPRILSLNNNPYFSGPYGEDV
VFVCNDWHTGPLSCYLKSNYQSHGIYRDAKTAFCIHNISYQGRFAFSDYP
ELNLPERFKSSFDFIDGYEKPVEGRKINWMKAGILEADRVLTVSPY
YAEE”

The present invention also provides starch association domain peptides and polypeptides which are more than 85% identical or homologous, or more than 90% identical or homologous or more than 95% identical or homologous, or more than 98% identical or homologous, or more than 99% identical or homologous, as compared with SEQ ID No. 1 Such sequences of the present invention may be obtained or derived, for example, from any of the noted crops or plants, or from any of the sequences of the NCBI or other similar database, such as for example any of gi 136757, 2833385, 136755, 136758, 2833382, 136765, 2833388, 267196, 6136121, 2833381, 2833383, 2833377, 2833387, 2829792, 2833390, 2833384, 729578, 2811062, 1169908, 1169909, 2829618, 729577, 2833389, 1174879, 140977 or 549804 or any present in SEQ ID No. 1 wherein a sequence similar or identical or homologous to any one of SEQ ID No. 1, within the embodiments of the presently described invention may be found.

The present invention further provides starch synthase enzymes, such as starch synthase I (SSI), starch synthase II (SSIIa or SSIIb) or starch synthase III (SSIII) wherein the region in the SSI, SSIIa, SSIIb or SSIII, corresponding to amino acids 103±at most 50 amino acids, to about amino acid 148±at most 50 amino acids of GBSS has been altered, modified or made to be more homologous or identical to the sequence spanning amino acids 103±at most 50 amino acids to about amino acid 148±at most 50 amino acids of GBSS. One of ordinary skill will appreciate that the homology or identity in his region between SSI, SSIIa, SSIIb or SSIII to GBSS is about 70-80% on average. By altering or modifying or engineering SSI, SSIIa, SSIIb or SSIII enzymes, for example, according to the present invention, to contain a starch association domain more similar to GBSS, starch synthases are provided which contain the advantageous glucan association properties of GBSS while retaining, at least substantially, the catalytic properties of the starch synthases, such as SSI, SSIIa, SSIIb or SSIII. These altered or modified or engineered peptides or polypeptides will be capable of producing or containing, for example, a greater percentage of continuous glucan sequences in an amylopectin cluster than produced by wild-type starch synthases, thus providing changes in the confirmational structure of the amylopectin clusters.

The present invention therefore provides starch synthase enzymes, other than GBSS, which contain a starch association domain as described above and herein which provides starch association properties similar to wild-type GBSS while preferably retaining the α-1,4 glucan transferase (i.e., catalytic properties) of soluble starch synthases, such as SSI, SSIIa, SSIIb and SSIII.

In a further embodiment, the present invention provides soluble starch synthase enzymes, such as SSI, SSIIa, SSIIb or SSIII, containing glucan association domain polypeptides or peptides which are more than 80% to 90% identical or homologous to the GBSS glucan association domain peptide or polypeptide, or homologous or identical, as defined above and herein, in the region of the starch synthase enzyme corresponding to the GBSS glucan association domain defined above and herein.

Such further glucan association domain peptides and polypeptides may be compared with GBSS starch association domains of the invention by means known in the art and described herein. Such soluble synthase glucan association domains include, for example, the sequences of gi 2833377, gi 2833387, gi 2829792, and gi 2833389 or those shown above, which were obtained from a BLAST search. Similar, homologous or identical polypeptide or peptide amino acid sequences are provided by the present invention.

In a manner similar to that described above wherein soluble starch synthase enzymes are provided which contain a glucan association domain similar to or the same as GBSS, the present invention also provides granule bound starch synthases which contain a soluble starch synthase or soluble starch synthase-like glucan association domain, which is preferably more than 80% to 90% identical or homologous to a soluble starch synthase glucan association domain. Such an altered or modified granule bound starch synthase will preferentially provide continuous glucan sequences in an amylopectin cluster, for example, which are, on average, shorter than provided with wild-type GBSS. The modified, altered or engineered GBSS of this embodiment of the present invention provides changes in confirmational structure of amylopectin structures and, likely, amylose structure.

The modified, altered or engineered granule bound starch synthases or soluble starch synthases may include glucan association domains of different species. That is, for example, the present invention provides maize granule bound starch synthases or maize soluble starch synthases which may contain a starch association domain region or sequence which is obtained or derived from, or at least 85% (or at least 90%, or at least 95%, or at least 98%, or at least 99%) homologous or identical to a starch association domain of Basella alba, for example. In this manner, the present invention provides granule bound starch synthases and soluble starch synthases wherein the starch association domain is obtained from, derived from or at least 85% (or at least 90%, or at least 95%, or at least 98%, or at least 99%) homologous or identical to starch association domain of any cereal, such as maize, wheat, rice, sorghum or barley; a fruit-producing species, such as banana, apple, tomato or pear; a root crop such as cassaya, potato, yam or turnip; an oilseed crop such as rapeseed, sunflower, oil palm, coconut, linseed or ground nut; a meal crop, such as soya, bean or pea; or any other suitable species.

The present invention also provides starch synthases, such as soluble starch synthases and granule bound starch synthases of Basella alba (Malabar spinach) that are found to have higher affinity to glucan substrates. Moreover, the present invention provides starch synthases, such as soluble starch synthases or granule bound starch synthases of species other than Basella alba, such as those described above, which have been engineered, modified or altered to contain at least one of the catalytic peptide or polypeptide sequence or the starch association domain peptide or polypeptide sequence of Basella alba or fragments, or homologous sequence, thereof, as described above.

### EXAMPLE I

### Expression of Fusion Proteins (Green Flourescent Protein (GFP), Metallothionein, and Citrate Synthase) Fused to Different GBSS Domains to Demonstrate that an Amino Acid Sequence of the Present Invention is Needed for Glucan Association of Expressed Recombinant Fusion Proteins

Affinity gel electrophoresis was used to demonstrate which one of the peptide domains of GBSS would associate with the glucan present in the native gels. For details on the Native gel electrophoresis, see below and the references listed herein as well as general knowledge in the art. The results of these experiments were compared with the genetic experiments by construction of plasmids carrying fusion proteins with different lengths of GBSS protein. Maize plants were transformed with the above said constructs. Transgenic plants containing the fusion protein were tested for both the levels of expression, and mainly the glucan (starch) association of the fusion protein. It was stunning that the peptide discovered from the biochemical experiments that had the glucan association properties was found to be the same that is required for glucan association of fusion proteins in transgenic maize plants.

The following constructs were made using fusion proteins of different lengths of GBSS protein and Green fluorescent protein (GFP), synthetic metallothinein and synthetic Pig heart citrate synthase. For the procedure on making constructs see below and herein as well as general knowldge in the art.

[image]

[image]

[image]

[image]

[image]

[image]

[image]

[image]

[image]

[image]

TABLE VI
Summary of the analysis of the Fusion Proteins made with different
domains of GBSS enzyme in maize kernels

| Plasmid | | Expressed | Is the enzyme | Entrapped in |
|---|---|---|---|---|
| Identification | Gene Construct | in soluble Fraction? | Active? | the granules? |
| I. | | | | |
| pEXS 206 | Transit peptide + GFP | Yes | NA | No |
| pEXS 208 | Transit peptide + GFP + (N−) truncated (−97bp) GBSS | Yes | NA | Yes |
| pEXS 210 | Transit peptide + GFP + full length GBSS | Yes | NA | Yes |
| pEXS 216 | Transit peptide + (N−)truncated (−702bp)GBSS + GFP | Yes | NA | No |
| pEXS 218 | Transit peptide + full length GBSS + GFP | Yes | NA | Yes |
| II. | | | | |
| pEXS 224 | Transit peptide + (N−)truncated (−300aa)GBSS + 1 × Metallothionein | N.D | NA | No |
| pEXS 228 | Transit peptide + (N−)truncated (−300aa)GBSS + 10 × Metallothionein | N.D | NA | No |
| III. | | | | |
| pEXS 233 | Transit peptide + (N−)truncated (−482aa)GBSS + Citrate synthase | Yes | YES | No |
| pEXS 234 | Transit peptide + (N−)truncated (−445aa)GBSS + Citrate synthase) | Yes | YES | No |
| pEXS 235 | Transit peptide + (N−)truncated (−395aa)GBSS + Citrate synthase | Yes | YES | No |

TABLE VI A.
Summary of Protein SEQ ID Nos:

| ENZYME | | | | | |
|---|---|---|---|---|---|
| and | | | | | |
| Protein | | | | | |
| Seq. | Transit | | | | |
| ID.No. | peptide | GLASS | LINKR | GLYTR | CTEND |
| GBSS-34 | 35-74 | 75-120 | 121-171 | 172-222 | 223-266 |
| SS1-267 | 268-283 | 284-335 | 336-386 | 387-437 | 438-461 |
| SSIIa-462 | 463-474 | 475-526 | 527-577 | 578-628 | 629-676 |
| SSIIb-677 | 678-681 | 682-732 | 733-783 | 784-834 | 835-882 |
| Du-I 883 | 884-932 | 933-982 | 983-1033 | 1034- | 1085- |
| | | | | 1084 | 1135 |

| | Seq.ID. | | Seq.ID. | | Seq.ID. |
|---|---|---|---|---|---|
| “GLASS” | No.s | “GLYTR” | No.s | “CTEND” | No.s |
| GBSS: | 1 | GBSS: | 1136 | GBSS: | 1146 |
| SSI: | 2 | SSl: | 1137 | SSI: | 1147 |
| SSIIa: | 3 | SSIIa: | 1138 | SSIIa: | 1148 |
| SSIIb: | 4 | SSIIb: | 1139 | SSIIb: | 1149 |
| SSIII (Dul): | 5 | SSIII (Du1): | 1140 | SSIII (Dul): | 1150 |

TABLE VI b
Summary of Nucleotide Seq. ID. NO. s.

| Nucleotide | sequence ID. No. s. |
|---|---|
| GBSS: | 1141 |
| SSI: | 1142 |
| SSIIa: | 1143 |
| SSIIb: | 1144 |
| SSIII (Dul): | 1145 |

TABLE VII
A Cartoon showing GBSS domain required for starch granular
entrapment (based on the transgenic analysis of various constructs
for entrapment of recombinant proteins)

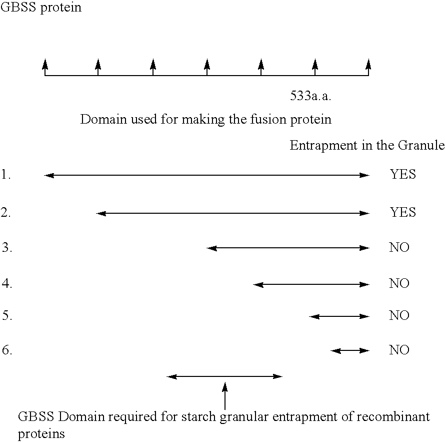

### EXAMPLES:

TABLE VIII
A Cartoon Showing the results from Biochemical evidence for
the GBSS peptide required for its association to a glucan
substrate in the gel.

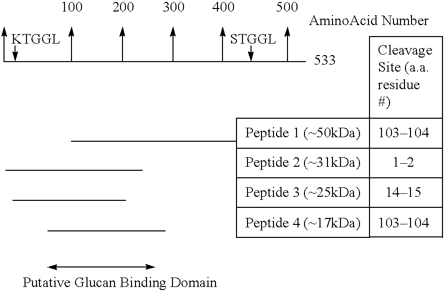

EXAMPLE I (See FIGS. 14 and 15) demonstrates the following:

1. Both biochemical and transgenic approaches identified the same peptide domain of GBSS as the Glucan Association Domain (Herein referred to as “GLASS” domain).Without presence of this particular domain, transgenic proteins did not associate with starch present in the endosperm of maize kernels. The “GLASS” domain is separate from glycosyl transferase domain (herein referred to as “GLYTR” domain in this patent). The order in which the proteins domains were fused did not matter for protein expression or glucan association as long as the “GLASS” domain was enclosed in the fusion protein. The present invention demonstrates that fusion protein technology of starch synthase enzymes may be applied in crop plants. Active fusion proteins were recovered with significant enzyme activity. The examples provided here demonstrate that the invention may be exemplified, without limitation, in maize crop.

### EXAMPLE II.

### Examples of Some Possible and Functional Fusion Proteins

The “GLASS” and GLYTR” domains of various SS and GBSS enzymes were fused and the 3D-models for the recombinant fusion proteins are provided. Using Protein threading onto 3D-PSSM, all the starch synthase enzymes from maize were very well comparable with highest confidence to bacterial UTDP-N-acetylglucosamine 2-epimerase. Campbell et al.2000,Biochemistry 39:14993-15001, determined the X-ray structure of UDP-N-acetylglucosamine 2-epimerase with bound UDP and identified a high degree of structural homology to glycogen phosphorylase, and T4-phage β-glycosyl transferases. The relatioship of epimerase to these glycosyl transferses is very intriguing and a similarity to starch synthases is proposed herein as the starch synthase enzymes have the same glycosyl transferase function. It is also very intriguing that, Pfam00534 (glycosyl transferse family, group 1) domain is universal across all the starch synthases tested. FIGS. 16 and 17 show structures of UDP-N-acetylglucosamine 2-epimerase and glycogen phosphorylase created using the same database (Kelley et al., 2000, J. of Mol. Biol.299: 499-520.

EXAMPLE II (See FIGS. 16 and 17) demonstrates the following:

Fusion proteins from examples provided above displayed 3D folding very similar to the native proteins in vivo. This was accomplished when proper peptide lengths of fusions were made from “GLASS” and “GLYTR” domains. The 3D-structure of starch synthases is more closely related to UDP-N-Acetylglucosamine 2-epimerase and T4 phageB-glucosyltransferase than to glycogen phosphorylase. Also, for any fusion protein, the presence of highly conserved “Pfam 00534” domain results in similar protein folding at 3D level. Glucan transfer takes place in the catalytic or “GLYTR” domain of the present invention. One of the functions of “GLASS” domain is glucan binding as in GBSS, but also the chain length specificity is within this domain as well.

### EXAMPLE III

Glucan Binding Properties of Starch Synthase Enzymes

Glucan affinity properties of various starch synthases (SS) enzymes from different plant species like banana fruit, basella leaf and carrot root, green bean pods, rice endosperm, rutabaga root, swetpotato root and wheat endosperm were examined and compared to maize endosperm SS forms. SSI enzyme from Basella alba displayed superior affinity to a given glucan (see table below) as compared to any of the maize enzymes studied so far. Therefore, the recombinant genes of SS enzymes from Basella and maize will enhance glucan-association properties of maize enzymes and thereby will result in better starch especially under adverse conditions. This transformation also results in altered amylopectin structure.

TABLE IX
A Comparison of K-values of Basella (B. alba L.) Starch
Synthase like Enzymes with Maize SSI enzyme

| Substrate | Enzyme | Temperature (4° C.) |
|---|---|---|
| Amylose | Basella-Band 1 | 0.035^{a} |
| | Basella-Band 2 | 0.284 |
| | Maize SSI-2 | 0.35 |
| Amylopectin | Basella-Band 1 | 0.002 |
| | Basella-Band 2 | 0.004 |
| | Maize SSI-2 | 0.06 |
| Glycogen | Basella-Band 1 | 0.0186 |
| | Basella-Band 2 | 0.112 |
| | Maize SSI-2 | 1.20 |
| Starch | Basella-Band 1 | 0.006 |
| | Basella-Band 2 | 0.036 |
| | Maize SSI-2 | 0.09 |

^{a}= Molar concentration is based on the average outer chain length (O.C.L.) of the substrate molecule (for amylose, amylopectin, and glycogen apparent average outer chain lengths are 8-9, 11-12, and 6-7, respectively).

A screen for starch synthase enzymes from different plant species and their affinities to glucan substrates was conducted. FIG. 18. shows SDS-electrophoresis and coomassie staining of proteins from various plants, namely banana fruit, basella leaf, carrot root, maize endosperm, green bean pods, rice endosperm, rutabaga root, sweetpotato root, and wheat endosperm. The proteins were run on native gel containing 2% boiled starch. The peptides or proteins that were bound to the glucan in the well were visualized by coomassie staining. And, were excised out of the native gel, and run on 10% SDS-gel. Very few peptides got bound to the glucan (data not shown). The proteins that were bound were transferred onto a nitrocellulose membrane for performing western blotting using maize SSI antibody. There was one protein in banana, two in basella, one in carrot, two or more in maize, one or two in green beans, two in ricen none in rutabaga and two in sweet potato, and two or three in wheat, were recognized by maize SSI antibody (Figure B). In order to confirm that these proteins that were bound to the glucan in the native gel and cross reacted with maize SSI antibody posses starch synthase activity, a renaturing gel was performed (see experimental procedures for details). These gels revealed both synthetic and degradative enzyme activity (Figure C) There were two proteins in banana, two in basella, one on corn, and two in wheat that possessed synthetic activity. Degradative enzyme activity was revealed in caroot, greenbean, sweetpotato and wheat (C). Figure D shows mobility of starch synthase enzymes of Basella alba in native gels containing no substrates (Controls). Also, starch synthase enzymes within maize endosperm have different affinities to glucans (See FIG. 19).

EXAMPLE III (See FIG. 19) demonstrates the following:

Various starch synthase enzymes have different affinities to a given glucan (FIG. 19). And, hence, it is possible to manipulate the functionality of native starch synthases and provide modifications to glucan chain lengths and starch structure.

The entire contents of the following references, along with the content of any references or documents or sequence deposit or other literature referred to and/or described herein or above or with the following are incorporated herein by reference in their entirety.

WO9720936 Starch Synthase Sequences

WO9844780 Starch Synthase Hosts

WO9814601 EnCapsulation

WO9924575 Dull1 Starch Synthase III

WO92/11376; 92/14827; 98/16190; 99/15636

PCT/GB92/01881

EPA 368506

U.S. Pat. Nos.: 5,792,920; 5,824,790; 5,859,333; 6,013,861; 6,107,060; 2,061,143; 4,789,557; 4,790,997; 4,774,328; 4,770,710; 4,798,735; 4,767,849; 4,801,470; 4,789,738; 9,30935;4,35,020; 4,792,458; 5,009,911; 5,300,145; 5,202,247; 5,137,819; 5,635,599; and 5,648,244.

UK Patent: 921818

Altschul, S. F., Madden, T. L., Schaffer, A. A., Zhang, J., Zhang, Z., Miller, W. and Lipmam, D. J. (1997) Gapped BLAST and PSI-BLAST: A new generation of protein data base search programs. Nucl. Acids Res. 25: 3389-3402.

Baba, T., Kimura, K., Mizuno, K., Etoh, H, Ishida, Y., Shida, O. and Arai, Y. 1991. Sequence conservation of the catalytic regions of amylolytic enzymes in maize branching enzyme I. Biochemical Biophysical Research Communication (BBRC) 181, 87-94

Baba, T., Nishihara, M., Mizuno, K., Kawasaki, T., Shimada, II., Kobayashi, E., Ohnishi, S., Tanaka, K. and Arai, Y. (1993) Identification, cDNA cloning, and gene expression of soluble starch synthase in rice (Oryza sativa L.) immature seeds. Plant Physiol 103, 565-573.

Baba, T., Yoshii, M. and Kainuma, K. (1987) Acceptor molecule of granule bound starch synthase from sweet-potato roots. Starch 39: 52-56.

Belshaw, N. J. and Williamson, G. (1993) Specificity of the binding domain of glucoamylase 1. Eur. J. Biochem. 211, 717-724.

Bhattacharyya, m. K., Smith, A. M., Ellis, T. H. N., Hedley, C., and Martin, C. (1990) The wrinkled seed character of pea described by Mendel is caused by transposan like insertion in a gene encoding starch branching enzyme. Cell, 60, 115-121.

Borovsky, D., Smith, E. E. and Whelan, W. J. (1976) On the mechanism of amylose branching by potato Q-enzyme. Eur. J. Biochem. 62, 307-312.

Boyer, C. D. and Preiss, J. (1979) Properties of citrate-stimulated starch synthesis catalyzed by starch synthase I of developing maize kernels. Plant Physiol. 64, 1039-1042.

Browner, M. F., Kakano, N., Bang, A. G., Fletterick, R. J. (1989) Human muscle glycogen synthase complementary DNA sequence: A negatively charged protein with an asymmetric charge distribution. Proc. Natnl.Acad. Sci (US) 86(5), 1443-1447.

Campbell R E. Mosimann S C. Tanner M E. Strynadka N C (2000). The structure of UDP-N-acetylglucosamine 2-epimerase reveals homology to phosphoglycosyl transferases. Biochemistry. 39(49):14993-15001.

Cao H., Imparl-Radosevich J., Guan H., Keeling, P. L., James, M. G. and Myers, A. M. (1999) Identification of the soluble starch synthase activities of maize endosperm. Plant Phys. 120, 205-215.

Cao, H., James, M. G., and Myers, A. M. (2000) Purification and characterization of soluble starch synthase from maize endosper. Arch. Biochem. BioPhys. 373(1), 135-146.

Chen, L, Ford, C., Nikolov, Z. (1991) Absorption to startch of β-galactosidase fusion protein containing the starch-binding region of aspergillus glucoamylase. Gene. 99: 121-126.

Chen, L., Coutinho, P. M., Nikolov, Z., Ford, C. (1995) Deletion analysis of the starch-binding domain of Aspergillus glucoamylase. Protein Engineering. 8: 1049-1055.

Commuri, P. D., and Keeling, Peter L. (2001) Chain-length specificities of maize starch synthase I enzyme: studies of glucan affinity and catalytic properties. The Plant Journal. 25: 475-486.

Commuri, P., et al (2002) Toward understanding the chain length specificities of starch synthase enzymes: Studies of glucan affinity and catalytic properties of maize granule bound starch synthase (GBSS). Will be Submitting to the Plant Journal.

Coutinho, P. M. and Reilly, P. J. (1994) Structure-function relationships in the catalytic and starch binding domains of glucoamylase. Protein Engineering. 7(3), 393-400.

Craig, J. Lloyd, J. R., Tomlinson, K., Barber, L., Edwards, A., Wang, T. L., Martin, C., Hedley, C. L. and Smith, A. M. (1998) Mutations in the gene encoding starch synthase II profoundly alter amylopectin structure in pea embryos. The Plant Cell. 10, 413-426.

Dalmia, B. K. and Nikolov, Z. L. (1994) Characterization of a β-galactosidase fusion protein containing the starch-binding domain of aspergillus glucoamylase. Enzyme Microbiology Technology. 16: 18-23.

Dalmia, Bipin K, Schuette, Kai, Nikolov, Sivko L., (1995) Domain E of Bacillus macerans cyclodextrin glucanotransferase: An independent starch-binding domain. Biotechnology and Bioengineering. 47(5): 575-584.

Dang,-P. L. and Boyer,-C. D. (1988) Maize leaf and kernel starch synthases and starch branching enzymes. Phytochemistry. 27 (5) p. 1255-1259.

Dang,-P. L. and Boyer,-C. D. (1989) Comparison of soluble starch synthases and branching enzymes from leaves and kernels of normal and amylose-extender maize. Biochem-Genet. 27 (9/10), 521-532.

Delrue, B., Fontaine, T., Routier, F., Decq, A., Wieruszeski, J. M., van der Koornhuyse, N., Maddelein, M. L., Fournet, B., and Ball, S. (1992) Waxy Chlamydonas reinhardtii: monocellular algal mutants defective in amylose biosynthesis and granule-bound starch synthase activity accumulate a structurally modified amylopectin. J. Bacteriol. 174, 3612-3620.

Denyer, K., Barber, L. M., Burton, R. et al. (1995) The isolation and characterization of novel low-amylose mutants of Pisum sativum L. Plant Cell Environ.18, 1019-1026.

Denyer, K., Clarke, B. and Smith, A. (1996) The elongation of amylose and amylopectin chains in isolated starch granules. Plant J. 10, 1135-1143.

Denyer, K., Johnson, P., Zeeman, S., Smith, A., M. (2001) The control of amylose synthesis. Journal of Plant Physiology. 158: 479-487.

Denyer, K., Sidebottom, C., Hylton, C. M. and Smith, A. M. (1993) Soluble isoforms of starch synthase and starch branching enzyme also occur within starch granules in developing pea embryos. Plant Journal. 4,191-198.

Denyer, K., Waite, D., Edwards, A., Martin, C. and Smith, A. M. (1999b) Interaction with amylopectin influences the ability of granule-bound starch synthase I to elongate malto-oligosaccharides. Biochem J. 342, 647-653.

Denyer, K., Waite, D., Motawia, S., Lindberg-Moller, B. and Smith, A. M. (1999a) Granule-bound starch synthase I in isolated starch granules elongates maltooligosaccharides processively. Biochem J. 340, 183-191.

Denyer,-K.; Smith,-A. M. (1992) The purification and characterization of the two forms of soluble starch synthase from developing pea embryos. Planta. 186 (4), 609-617.

Dry, I., Smith A., Edwards, A., Bhattacharyya, M., Dunn, P., and Martin, P. (1992) Characterization of cDNAs encoding two isoforms of granule-bound starch sytnhases which show differential expression in developing storage organ of pea and potato. Plant J.2, 193-202.

Dubois, M., Gilles, K. A., Hamilton, J. K., Rebers, P. A. and Smith, F. (1956) Colorimetric method for determination of sugars and related substances. Analytical chem. 28 (3), 350-356.

Edwards, A., Fulton, D.C., Hylton, C. M., Jobling, S. A., Gidley, M., Rossner, U., Martin, C., and Smith, A. M. (1999) A combined reduction in activity of starch synthases II and III of potato has novel effects on the starch of tubers. Plant Journal. 17: 251-261.

Edwards, A., Marshall, J., Denyer, K., Sidebottom, C., Visser, R. G. F., Martin, C. and Smith, A. M. (1996) Evidence that a 77-Kilodalton protein from the starch of pea embryos is an isoform of starch synthase that is both soluble and granule bound. Plant Physiol. 112, 89-97.

Edwards, A., Marshall, J., Sidebottom,C., Visser, R. G. F., Smith, A. M. and Martin, C. (1995) Biochemical and molecular characterization of a novel starch synthase from potato tubers. The Plant Journal. 8(2), 283-294.

Fisher D. K., Kim, K. N., Gao, M., Boyer, C. D. and Guiltinan, M. J. (11995) A cDNA encoding starch branching enzyme I from maize endosperm. Plant Physiol. 108, 1313-1314.

Fisher, D. K., Boyer, C. D. and Hannah, L. C. (1993) Starch branching enzyme 11 from maize endosperm. Plant Physiol. 102, 1045-1046.

Flipse, E., Keetels, C. J. M., Jacobsen, E., Visser, R. G. F. (1996) The dosage effect of the wild type GBSS allele is linear for GBSS activity but not for amylose content: Absence of amylose has a distict influence on the physico-chemical properties of starch. Theor. Appl. Genet. 92: 121-127.

Frydman, R. B. and Cardini C. E. (1967) Studies on the biosynthesis of starch II. Some properties of the adenosine diphosphate glucose. J. Biol. Chem. 242. 312-317.

Furukawa K. Tagaya M. Tanizawa K. Fukui T.(1993) Role of the conserved Lys-X-Gly-Gly sequence at the ADP-glucose-binding site in Escherichia coli glycogen synthase. Journal of Biological Chemistry. 268(32):23837-42.

Furukawa, K., Tagaya, M., Inouye, M., Preiss, J., and Fukui, T. (1990) Identification of lysine 15 at the active site in Escherichia coli glycogen synthase. J. Biol. Chem 265, 2086-2090.

Gao, M., Chibber, R. N., Isolation, and expression analysis of starch synthase IIa cDNA from wheat (Triticum aestivum L.)¹. NRC Canada. 43: 768-775.

Gao, M., Fisher, D. K., Kim, K. N., Shannon, J.C. and Guiltinan, M. J. (1996) Evolutionary conservation and expression patterns of maize starch branching enzyme I and II b genes suggests isoform specialization. Plant Mol. Biol. 30, 1223-1232.

Gao, M., Fisher, D. K., Kim, K. N., Shannon, J. C. and Guiltinan, M. J. (1997) Independent genetic control of maize starch-branching enzymes Ia and IIb. Plant Physiol. 114, 713-722.

Gao, M., Wanat, J., Stinard, P.S., James, M. G. and Myers, A. M. (1998) Characterization of dull1, a maize gene coding for a novel starch synthase. The Plant Cell. 10, 399-412.

Gao, Z. (2001) The structure-function relationships of maize starch synthase. Ph.D. Thesis. Iowa State University, Ames, Iowa.

Goto, Seminaru, Furukawa, Hayashida (1994) Analysis of the raw starch-binding domain by mutation of a glucoamylase from aspergillus awamori var. kawachi expressed in saccharomyces cerevisiae. Applied and Environmental Microbiology. 60: 3926-3930.

Guan, H. and Keeling, P. L. (1998) Understanding the functions and interactions of multiple isozymes of starch synthase and branching enzyme. Trends in Glycoscience. 10, 307-319.

Ham, C., Knight, M., Ramakrishnan, A., Guan H., Keeling, P. L. and Wasserman, B. P. (1998) Isolation and characterization of the zSSIIa and zSSIIb starch synthase cDNA clones from maize endosperm. Plant Molecular Biology. 37, 639-649.

Hawker, J. S. and Jenner, C. F. (1993) High temperature affects the activity of enzymes in the committed pathway of starch synthesis in developing wheat endosperm. Aust J. Plant Physiol. 20, 197-209.

Heijne, G. von. (1991) CHLPEP—a database of chloroplast transit peptides. 9(2): 104-126.

Hizukuri, S., Takeda, Y. and Yasuda, M. (1981) Multi-branched nature of amylose and the action of de-branching enzymes. Carbohydrate Research. 94, 205-213.

Hizukuri, S., Takeda, Y., Maruta, N. and Julian, B. O. (1989) Molecular structures of rice starch. Carbohydrate Research. 189, 227-235.

Hovenkamp-Hennelink,-J. H. M.; Jacobsen,-E.; Ponstein,-A. S.; Visser.-R. G. F.; Vos-Scheperkeuter,-G. H.; Biimolt,-E. W.; De-Vries,-J. N.; Witholt,-B.; Feenstra,-W. J. (1987) Isolation of an amylose-free starch mutant of the potato (Solanum tuberosum L.). Theor-Appl-Genet. 75 (1), 217-221.

Imparl-Radosevich, J. M., Li, P., Zhang, L., McKean, A. L., Keeling, P. L. and Guan, H. (1998a) Purification and characterization of maize starch synthase I and its truncated forms. Archives of Biochem and Biophysics. 353 (1), 64-72.

Imparl-Radosevich, J. M., Nichols, D. J., Li, P., McKean, A. L., Keeling, P. L. and Guan, H. (1998b) Analysis of purified maize starch synthases IIa and IIb: SS isoforms can be distinguished based on their kinetic properties. Archives of Biochem and Biophysics. 362 (1),131-138.

Jane, J. and Chen, J. (1992) Effect of amylose molecular size and amylopectin branch chain length on paste properties of starch. Cereal Chem. 69(1), 60-65.

Janecek S., and Sivcek, J. (1999) The evolution of starch-binding domain. FEBS Letters. 456: 119-125.

Jenner, C. F., Denyer, K. and Guerin, J. (1995) Thermal characteristics of soluble starch synthase from wheat endosperm. Aust. J. Plant Physiol. 22, 703-709.

Jenner, C. F., Siwek, K. and Hawker, J. S. (1993) The synthesis of 14C starch from 14C sucrose in isolated wheat grains is dependent upon the activity of soluble starch synthase. Aust. J. Plant Physiol. 20, 329-335.

Kainuma K. (1988) Structure and chemistry of the starch granule. In: Preiss, J. (Ed.), The Biochemistry of Plants, Vol 14. Academic Press, San Diego, pp. 141-180.

Keeling, P. L., Bacon, P. J. and Holt, D.C. (1993) Elevated temperature reduces starch deposition in wheat endosperm by reducing the activity of soluble starch synthase. Planta. 191, 342-348.

Keeling, P. L., Banisadr, R., Barone, L., Wasserman, B. P. and Singletary, G. W. (1994) Effect of temperature on enzymes in the pathway of starch biosynthesis in developing wheat and maize grain. Aust. J. Plant Physiol. 21, 807-827.

Kelley, L. A., MacCallum, R. M., Sternberg, M. J. E. (2000) Enhanced Genome Annotation Using Structural Profiles in the Program 3D-PSSM. Journal of Molecular Biology. 299: 499-520.

Kiel, J. A., Elgersma, H. S., Beldman, G., Vossen, J. P., Venema, G. (1989) Cloning and expression of the branching enzyme gene (glgB) from the cyanobacterium Synechococcus sp. PCC7942 in Escherichia coli. Gene. 78(1):9-17May 15

Kiel, J. A., Boels, J. M., Beldman, G., Venema, G. (1991) Molecular cloning and nucleotide sequence of the glycogen branching enzyme gene (glgB) from Bacillus stearothermophilus and expression in Escherichia coli and Bacillus subtilis. Molecular & General Genetics. 230(1−2):136-44. Kiel, J. A. K. W., Elergsma, H. S. A., Beldman, G, Vossen, J. P. M. J, and Venema, G (1991): Cloning and expression of the branching enzyme gene (glgB) from the cyanobacterium Synechococcus sp. PCC7942 in Escherichia coli. Gene (AMSTERDAM) 78(1), 9-18.

Knight, E., Ham, C., Lilley, C. E. R., Guan, H., Singletary, G. W., Mu-Forster, C., Wasserman, B. P. and Keeling, P. L. (1998) Molecular cloning of starch synthase from maize (W64) endosperm and expression in Escherichia coli. Plant J. 14, 613-622

Kossmann, J. (1991) Cloning and expression analysis of a potato cDNA that encodes branching enzyme: ecidence for co-expression of starch biosynthetic genes. Mol. Gen. Genet. 230(12): 39-44.

Kumar, A., Larson. C. E., and Preiss, J. (1986) Biosynthesis of α-1,4 glucan, 4-glucosyltransferase as deduced from the nucleotide sequence of the glgA gene. J. Biol. Chem. 261, 16256-16259.

Kusnadi, A. R., Ford, C. and Nikolov, Z. L. (1993) Functional starch-binding domain of Aspergillus glucoamylase I in Escherichia coli: Maltose-binding protein; fusion protein;

Laemmli, U.K. (1970) Cleavage of structural proteins during the assembly of the head of bacteriophage T4. Nature. 227, 680-685.

Leij, F. R. van der. (1991) Complementation of the amylose-free starch mutant of potato (Solanum tuberosum) by the gene encoding granule-bound starch synthase. 82(3): 289-295.

Leloir, L. F., De Feketa, M. A. R., and Cardini C. E. (1961) Starch and oligosaccharide synthesis from uridine diphosphate glucose. J. Biol. Chem. 236, 636-641.

Leung, P. S. C. and Preiss, J. (1987) Cloning of the ADP glucose pyrophosphorylase (glgC) and glycogen synthase (glgA) structural genes from Salmoniella typhimurium LT2. Jounal of Bacteriology 169(9), 4349-4354.

Li, Z., Rahmnan, S., Kosar-Haskemi, B., Mouille, G., Appels, R. and Morell, M. K. (11999) Cloning and characterization of a gene encoding wheat starch synthase I. Theor Appl Genet. 98, 1208-1216.

Lloyd, J. R., Landschutze, V, and Kossmann, J. (1999) Simultaneous antisense inhibition of two starch-synthase isoforms in potato tubers to accumulation of grossly modified amylopectin. Biochem J. 338, 515-521

Lu, T. J., Jane, J. L., Keeling, P. L., and Singletary, G. W. (1996) Maize starch fine structures affected by ear developmental temperature. Carbo.Res. 282, 157-170.

Macdonald,-F. D. and Preiss,-J. (1985) Partial purification and characterization of granule-bound starch synthases from normal and waxy maize. Plant-Physiol. 78 (4) p. 849-852.

Macdonald,-F. D. and Preiss,-J. (1986) The subcellular location and characteristics of pyrophosphate-fructose-6-phosphate 1-phosphotransferase from suspension-cultured cells of soybean. Planta. 167 (2), 240-245.

Maddelein, M. L., Libessart, N., Bellanger, F., Delrue, B., D'Hulst C. (1994) Towards an understanding of the biogenesis of the starch granule: determination of granule bound and soluble starch synthase functions in amylopectin synthesis. J. Biol. Chem. 269, 25150-25157.

Marshall, J., Sidebottom, C., Debet, M., Martin, C., Smith, A. M. and Edwards, A. (1996) Identification of the major starch synthase in the soluble fraction of potato tubers. The Plant Cell 8, 1121-1135

Martin, Smith (1995) Starch Biosynthesis. The Plant Cell. 7: 971-985.

Matsumoto, A., Nakajima, T. and Matsuda, K. (1990) A kinetic study between the interaction of glycogen and Neurospora crassa branching enzyme. J. Biochem. 107(1), 123-126

Mu, C., Harn, C., Ko, Y. T., Singletary, G. W., Keeling, P. L. and Wasserman, B. P. (1994) Association of a 76 kDa polypeptide with soluble starch synthase I activity in maize (cvB73) endosperm. Plant J. 6, 151-159

Mu-Forster, C., Huang, R., Powers, J. R., Harriman, R. W., Knight, M., Singletary, G. W., Keeling, P. L. and Wasserman, B. P. (1996) Physical association of starch biosythetic enzymes with starch granules of maize endosperm: Granule-associated forms of starch synthase I and starch branching enzyme II. Plant Physiol. 111, 821-829.

Myers, A. M., Morell, M. K., James, M. G. and Ball, S. G. (2000) Recent progress toward understanding biosynthesis of the amylopectin crystal. Plant Physiol. 122, 989-997.

Nakajima, R., Imanaka, T. and Aiba, S. (1986) Comparison of amino acid sequences of eleven different a-amylases. Appl. Microbiol. Biotechnol. 23, 355-360 Nakamura, Y., Takeichi, T., Kawaguchi, K., Yamanouchi, H. (1992) Purification of two forms of starch branching enzyme (Q-enzyme) from developing rice endosperm. Physiol. Plantarum 84(3), 329-335.

Nakamura,-Y. and Yamanouchi,-H. (1992) Nucleotide sequence of a cDNA encoding starch-branching enzyme, or Q-enzyme I from rice endosperm. Plant-Physiol. 99 (3) p. 1265-1266.

Namnori, T., Nagai, M., Shimizu, Y., Shinke, R. and Mikami, B. (1993) Cloning of the B-amylase Igene from Bacillus cereus and characteristics of the primary structure of the enzyme. Applied & Environmental Microbiology. 59(2), 623-627

Okita, T. W. and Preiss, J. (11981) Starch degradation in spinach leaves: Isolation and characterization of the amylases and R-enzyme of spinach leaves. Plant Physiol.66(5): 870-876.

Okita, T. W. and Preiss,J. (1980) Starch degradation in spinach leaves: isolation and characterization of the amylases and R-enzyme of spinach leaves. Plant-Physiol. 66 (5) 870-876.

Okita-T-W; Rodriguiez-R-L; Preiss-J (1982) Biosynthesis of bacterial glycogen: Cloning of the glycogen biosynthetic enzyme structural genes of Escherichia coli. J. Biol. Chem. 256 (13) 6944-6952.

Ong, E. (1993) Proteins designed for adherence to cellulose. American Chemical Society. 516: 185-194.

Pollock, C. and Preiss, J. (1980) The citrate-stimulated starch synthase of starchy maize kernels; purification and properties. Plant Physiol. 204(2), 578-588.

Preiss,-J.; MacDonald,-F. D.; Singh,-B. K.; Robinson,-N.; McNamara,-K. (1985) Various aspects in the regulation of starch biosynthesis. Prog-Biotechnol. 1, 1-17.

Reddy, K. R., Ali, S. Z. and Bhattacharya, K. R. (1993) The fine structure of rice starch amylopectin and its relation to the texture of cooked rice. Carbohydrate Polymers, 22, 267-275

Rowen, D. W., Meinke, M., Laporte, D. C. (1992) GLC3 and GHA1 of Saccharomyces cerevisiae are allelic and encode the glycogen branching enzyme. Molecular and Cell Biol. 12 (1): 22-29.

Salehuzzaman,-S. N. I. M.; Jacobsen,-E.; Visser,-R. G. F. (1993) Isolation and characterization of a cDNA encoding granule-bound starch synthase in cassaya (Manihot esculenta Crantz) and its antisense expression in potato. Plant-mol-biol.23 (5), 947-962.

Salehuzzaman,-S. N. L. M.; Jacobsen,-E.; Visser,-R. G. F. (1992) Cloning, partial sequencing and expression of a cDNA coding for branching enyzme in cassaya. Plant-Mol-Biol-Int-J-Mol-Biol-Biochem-Genet-Eng. 20 (5), 809-819.

Sambrook, J., Fritsch, E. F., Maniatis, T. (1989) Molecular Cloning: A Laboratory Manual. 2^{nd} edition. Vols 1-3. Cold Spring Harbor Laboratory Press.

Shimada, H. (1993) Antisense regulation of the rice waxy gene expression using a PCR-amplified fragment of the rice genome reduces the amylose content in grain starch. Theor. Appl. Genet. 86(6): 665-672.

Shure, M., Wessler, S. and Fedoroff, N. (1983) Molecular identification and isolation of the waxy locus in maize. Cell. 35, 225-233.

Siggens, K. (1987) Molecular cloning and characterization of the B-amylase gene from Bacillus circulans. Mol. Microbiology 1, 86-91.

Singh,-B. K. and Preiss, J. (1985) Starch branching enzymes from maize. Immunological characterization using polyclonal and monoclonal antibodies. Plant-Physiol. 79 (1), 34-40.

Smith, A. M. (1988) Major differences in isoforms of starch-branching enzyme between developing embryos of round- and wrinkeld-seeded peas [Pisum sativum L.] Planta. 175(2), 270-279.

Smith, A. M., Denyer, K. and Martin, C. (1997) The synthesis of the starch granule. Annu. Rev. Plant Physiol. Plant Mol. Biol. 48, 67-87.

Smith,-A. M.; Martin,-C.; Denver,-K. (1991) The pathway of starch synthesis in developing pea embryos. Trans-Biochem-Soc. 19 (3), 547-550.

Sôgaard, M., Kadziola, A., Haser, R. and Svensson, B. (1993) Site-directed mutagenesis of histidine 93, aspartic acid 180, glutamic acid 205, histidine 290, and aspartic acid 291 at the active site and trytophan 279 at the raw starch binding site in barley-amylase 1. J. of Biological Chemistry. 268 (30), 22480-22484.

Svensson, B. (1988) Regional distant sequence homology between amylases, a-glucosidases and transglucosylases. FEBS Lett. 230 (1,2), 72-76.

Svensson, B., Jespersen, H., Sierks, M. R., MacGregor, E. A. (1989) Sequence homology between raw-starch binding domains from different starch-degrading enzymes. Biochemical Journal. 264: 309-311.

Takeda, Y. and Preiss, J. (1993) Structures of B90 (sugary) and W64A (normal) maize starches. Carbohydrate Res. 240, 265-275.

Takeda, Y., Guan, H. and Preiss, J. (1993) Branching of amylose by the branching isoenzymres of maize endosperm. Carbohydrate Res. 240, 253-263.

Takeo, K. and Nakamura, S. (1972) Dissociation constants of glucan phosphorylases of rabbit tissues studied by polyacrylamide gel disc electrophoresis. Arch. Biochem. Biophys. 153, 1-7

Tsai,-C-Y. (1974) Sucrose UDP glucosyltransferase of Zea mays endosperm. [Corn] Phytochemistry 13 (6), 885-891.

Tsai,C-Y. (1974) The function of the waxy locus in starch synthesis in maize endosperm. Biochem-Genet, 11 (2), 83-96.

Tsai,C-Y. and Dalby,A. (1974) Comparison of the effect of shrunken-4, opaque-2, opaque-7, and floury-2 genes on the zein content of maize during endosperm development. Cereal-Chem, 51 (6): 707-717.

Tuomi,-T.; Tanskanen,-J.; Tyynela,-J.; Terri,T. H.; Schulman,-A. H. (1993) Towards in planta modification of starch synthesis. Asp-appl-biol. 36, 93-101.

Tyneela, J and Schulman, A. H. (1993) An analysis of soluble starch synthase isozymes from the developing grains of normal and shx cv. Bomi barley (Hordeum vulgare). Physiologia-Plantarum. 89 (4), 835-841.

Visser, R. G. F., Hergersberg, M., van der Leij, F. R., Jacobsen, E., Witholt, B., Feenstra, W. J. (1989) Molecular cloning and partial characterization of the gene for granule-bound starch synthase from a wild and an amylose-free potato. Plant Sci. 64: 185-192.

Visser,-R. G. F., Hesseling-Meinders.A., Jacobsen, E., Nijdam, H., Witholt,B. and Feenstra, W. J. (1989) Expression and inheritance of inserted markers in binary vector carrying Agrobacterium rhizogenes-transformed potato (Solanum tuberosum L.).Theor-Appl-Genet. 78 (5), 705-714.

Visser,R. G. F., Jacobsen,E. Witholt,B. and Feenstra, W. J. (1989) Efficient transformation of potato (Solanum tuberosum L.) using a binary vector in Agrobacterium rhizogenes. Theor-Appl-Genet. 78 (4), 594-600.

Visser, R. G. F., Somhorst, I., Kuipers, G. J., Ruys, N. J., Feenstra, W. J. and JacobsenE.(1991) Inhibition of the expression of the gene for granule-bound starch synthase in potato by antisense constructs. M-G-G-Mol-Gen-Genet. 225 (2), 289-296.

Visser,-R. G. F.; Hoekstra,-R.; Leij,-F. R.-van-der; Pijnacker,-L. P.; Witholt,-B.; Feenstra,-W. J. (1988) In situ hybridization to somatic metaphase chromosomes of potato. Theor-Appl-Genet. 76 (3) p. 420-424.

Vos-Scheperkeuter.-G. H.; Boer,-W.-de; Visser,-R. G. F.; Feenstra,-W. J.; Witholt,-B. (1986) Identification of granule-bound starch synthase in potato tubers. Plant-Physiol. 82 (2), 411-416.

Vos-Scheperkeuter,-G. H.; Wit,-J. G.-de; Ponstein,-A. S.; Feenstra,-W. J.; Witholt,-B. (1989) Immunological comparison of the starch branching enzymes from potato tubers and maize kernels. Plant-Physiol. 90 (1), 75-84.

SEQ. ID. No. 1
Glucan Association Domain (“GLASS) of GBSS
“K I Y G P V A G T D Y R D N Q L R F S L L C Q A A L E A P R I L S L N N N P
Y F S G P Y G E D V V F V C N D W H T G P L S C Y L K S N Y Q S H G I Y R D A
K T A F C I H N I S Y Q G R F A F S D Y P E L N L P E R F K S S F D F I D G Y
E K P V E G R K I N W M K A G I L E A D R V L T V S P Y Y A E E“
Seq ID. No. 2
Glucan Association domain (“GLASS”)domain (”GLASS”) of maize SSI
E G I A E G S I D N T V V V A S E Q D S E I V V G K E Q A R A K V T Q S I V F
V T G E A S P Y A K S G G L G D V C G S L P V A L A A R G H R V M V V M P R Y
L N G T S D K N Y A N A F Y T E K H I R I P C F G G E H E V T F F H E Y R D S
V D W V F V D H P S Y H R P G N L Y G D K F G A F G D N Q F R Y T L L C Y A A
C E A P L I L E L G G Y I Y G Q N C M F V V N D W H A S L V P V L L A A K Y R
P Y G V Y K D S R S I L V I H N L A H Q G V E P A S T Y P D L G L P P E W Y G
Seq ID. No. 3
Glucan Association domain (“GLASS”)domain (“GLASS”) of maize SSIIa
S K R R D P L Q P V G R Y G S A T G N T A R T G A A S C Q N A A L A D V E I K
S I V A A P P T S I V K F P A P G Y R M I L P S G D I A P E T V L P A P K P L
H E S P A V D G D S N G I A P P T V E P L V Q E A T W D F K K Y I G F D E P D
E A K D D S R V G A D D A G S F E H Y G D N D S G P L A G E N V M N V I V V A
A E C S P W C K T G G L G D V V G A L P K A L A R R G H R V M V V V P R Y G D
Y V E A F D M G I R K Y Y K A A G Q D L E V N Y F H A F I D G V D F V F I D A
P L F R H R Q D D I Y G G S R Q E I M K R M I L F C K V A V E V P W H V P C G
G V C Y G D G N L V F I A N D W H T A L L P V Y L K A Y Y R D H G L M Q Y T R
S V L V I H N I A H Q G R G P V D E F P Y M D L P E H Y L Q H F E L Y D P
Seq ID. No.4
GlucanAssociation domain (“GLASS”)domain (“GLASS”) of maize SSIIb
A D A A P A T D A A A S A P Y D R E D N E P G P L A G P N V M N V V V V A S E
C A P F C K T G G L G D V V G A L P K A L A R R G H R V M V V I P R Y G E Y A
E A R D L G V R R R Y K V A G Q D S E V T Y F H S Y I D G V D F V F V E A P P
F R H R H N N I Y G G E R L D I L K R M I L F C K A A V E V P W Y A P C G G T
V Y G D G N L V F I A N D W H T A L L P V Y L K A Y Y R D N G L M Q Y A R S V
L V I H N I A H Q G R G P V D D F V N F D L P E H Y I D H F K L Y D N I G
Seq ID. No. 5
GlucanAssociation domain (“GLASS”) of maize Du1
G G I Y D N R N G L D Y H I P V F G S I A K E P P M H I V H I A V E M A P I A
K V G G L G D V V T S L S R A V Q D L G H N V E V I L P K Y G C L N L S N V K
N L Q I H Q S F S W G G S E I N V W R G L V E G L C V Y F L E P Q N G M F G V
G Y V Y G R D D D R R F G F F C R S A L E F L L Q S G S S P N I I H C U D W S
S A P V A W L H K E N Y A K S S L A N A R V V F T I H N L E
SEQ. ID. No. 6
SSI
Sequence used by 3DPSSM for threading

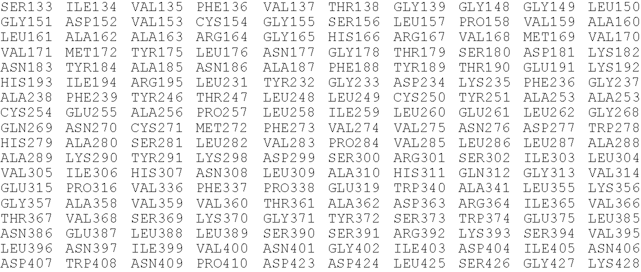

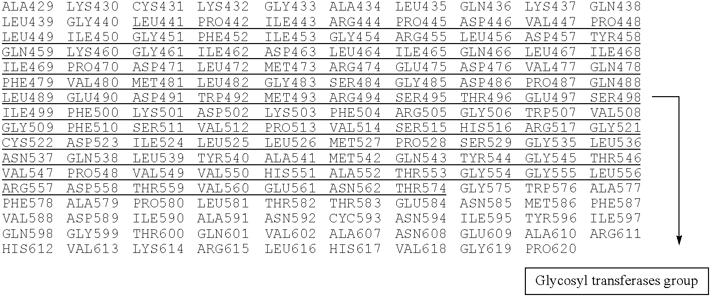

| Secondary Structure | Calculated |
|---|---|
| Number of Groups | 349 |
| Number of Atoms | 2708 |
| Number of Bonds | 2791 |
| Number of H-Bonds | 237 |
| Number of Helices | 20 |
| Number of Strands | 17 |
| Number of Turns | 30 |

SEQ. ID. No. 7
SSIIa
Sequence utilized by 3DPSSM

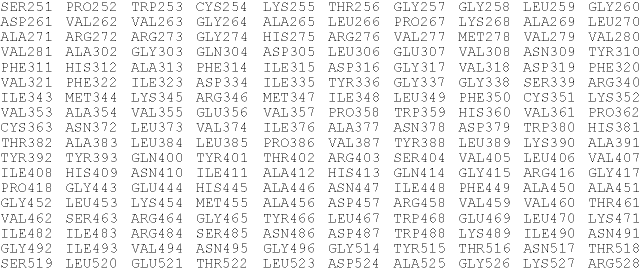

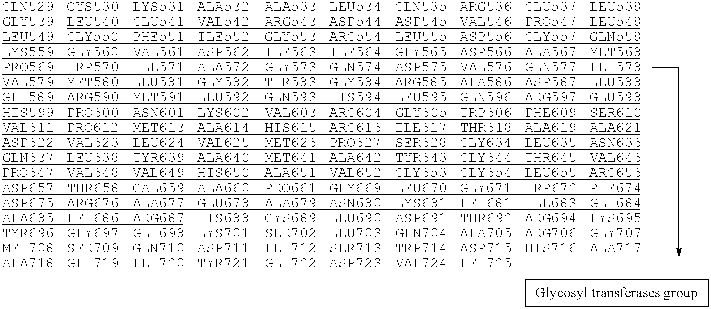

| Secondary Structure | Calculated |
|---|---|
| Number of Groups | 358 |
| Number of Atoms | 2802 |
| Number of Bonds | 2885 |
| Number of H-Bonds | 246 |
| Number of Helices | 22 |
| Number of Strands | 17 |
| Number of Turns | 30 |

SEQ. ID. No. 8
SSIIB-
Sequence used by 3DPSSM for threading

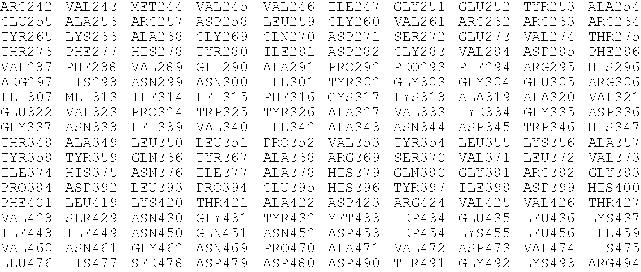

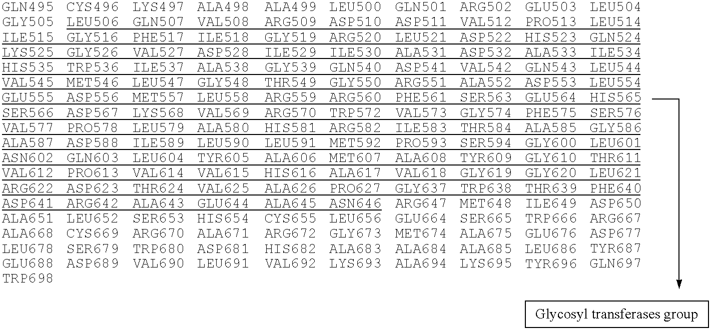

| SecondaryStructure | Calculated |
|---|---|
| Number of Groups | 361 |
| Number of Atoms | 2888 |
| Number of Bonds | 3012 |
| Number of H-Bonds | 252 |
| Number of Helices | 22 |
| Number of Strands | 17 |
| Number of Turns | 31 |

SEQ. ID. No. 9
GBSS
GBSS Sequence used by 3D-PSSM

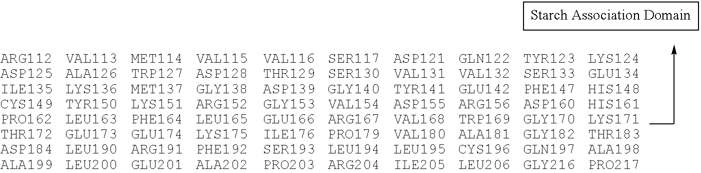

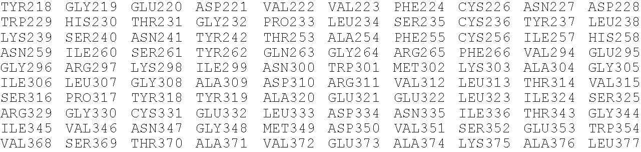

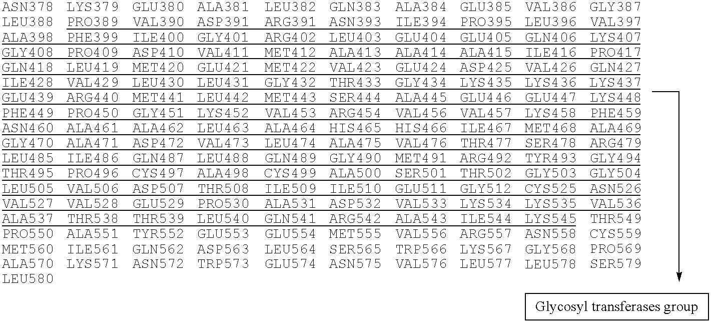

| SecondaryStructure | Calculated |
|---|---|
| Number of Groups | 361 |
| Number of Atoms | 2805 |
| Number of Bonds | 2877 |
| Number of H-Bonds | 252 |
| Number of Helices | 21 |
| Number of Strands | 17 |
| Number of Turns | 30 |

SEQ. ID. No. 10
Du I
DuI sequence used by 3D-PSSM
Note: Residue numbered as 37 (listed below) is actually amino acid residue
number 1238 in Du1 protein sequence

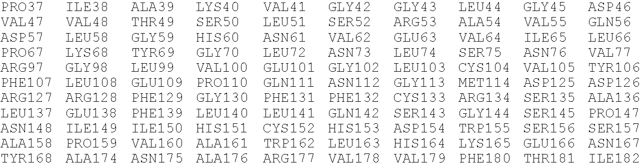

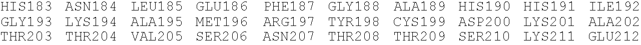

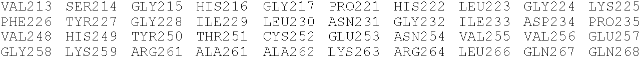

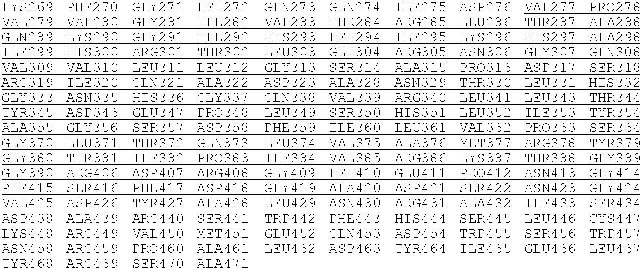

TABLE III

| | | | | | | % |
|---|---|---|---|---|---|---|
| | | | | | | align- |
| ENZYME | Seq. ID No. | Alignment with Pfam 00534 (Glycosyl transferase, Group 1 domain) | | | | ment |
| GBSS | Seq. Id. | query: | 378 | NKEALQAEVGLPVDRNIPLVAFIGRLEEQKGPDVMAAAIPQLME-MVEDVQIVLLGTGKK | 436 | 91.3 |
| | No.11 (query) | | | | | |
| | Seq. Id. | Sbjct: | 1 | DREEIRKKLGIKEEKKI--ILFVGRLLPEKGIDLLIEAFKKLKKQLNPNLKLVIVGDGEE | 58 | |
| | No.12 (snjct) | | | | | |
| | | Query: | 437 | KFERMLMSAEEKFPGKVRAVVKFNAALAHHIMAGADVLAVTSRFEPCGLIQLQGMRYGTP | 496 | |
| | | Sbjct: | 59 | EDELKLLALKLGLEDNVIFLGFVPDEDLPELYKSADVFVLPSRYEGFGIVLLEAMACGLP | 118 | |
| | | Query: | 497 | CACASTGGLVDTIIEGKTGFHMGRLSVDCNVVEPADVKKVATTLQRAIK 545 | | |
| | | Sbjct: | 119 | VIATDVGGIPEIVKDGETGL----------LVEPGDVEALAEAIEKLLK 157 | | |
| SSI | Seq. Id. | Query: | 441 | LPIRPDVPLIGFIGRLDYQKGID-LIQLI--IPDLMREDVQFVMLGSGDPELEDWMRSTE | 497 | 75 |
| | No.13 (query) | | | | | |
| | Seq. Id. | Sbjct: | 9 | LGIKEEKKIILFVGRLLPEKGIDLLIEAFKKLKKQLNPNLKLVIVGDGEEEDELKLLALK | 68 | |
| | No.14-(snjct) | | | | | |
| | | Query: | 498 | SIFKDKFRGWVGF-SVPVSHRITAGCDILLMPSRFEPCGLNQLYAMQYGTVPVVHATGGL | 556 | |
| | | Sbjct: | 69 | LGLEDNVI-FLGFVPDEDLPELYKSADVFVLPSRYEGFGIVLLEAMACGLPVIATDVGGI | 127 | |
| | | Query: | 557 | RDTVENFNPFGENG 570 | | |
| | | Sbjct: | 128 | PEIVKD----GETG 137 | | |
| SSIIa | Seq. Id. | Query: | 540 | LEVRDDVPLLGFIGRLDGQKGVDIIGDAMPWIA---GQDVQLVMLGTGRADLERMLQHLE | 596 | 84.3 |
| | No.15 (query) | | | | | |
| | Seq. Id. | Sbjct: | 9 | LGIKEEKKIILFVGRLLPEKGIDLLIEAFKKLKKQLNPNLKLVIVGDGEEEDELKLLALK | 68 | |
| | No.16 (snjct) | | | | | |
| Query: | 597 | REHPNKVRGWVGFSVPMAHRIT--AGADVLVMPSRFEPCGLNQLYAMAYGTVPVVHAVGG | 654 | | | |
| | | Sbjct: | 69 | LGLEDNVI-FLGF-VPDEDLPELYKSADVFVLPSRYEGFGIVLLEAMACGLPVIATDVGG | 126 | |
| | | Query: | 655 | LRDTVAPFDPFGDAGLGWTFDRAEANKLIEALR 687 | | |
| | | Sbjct: | 127 | IPEIVKDGET------GLLVEPGDVEALAEAIE 153 | | |
| SSIIb | Seq. Id. | Query: | 506 | LQVRDDVPLIGFIGRLDHQKGVDIIADAIHWIA---GQDVQLVMLGTGRADLEDMLRRFE | 562 | 87.2 |
| | No.17 (query) | | | | | |
| | Seq. Id. | Sbjct: | 9 | LGIKEEKKIILFVGRLLPEKGIDLLIEAFKKLKKQLNPNLKLVIVGDGEEEDELKLLALK | 68 | |
| | No.18 (snjct) | | | | | |
| | | Query: | 563 | SEHSDKVRAWVGFS----VPLAHRITAGADILLMPSRFEPCGLNQLYAMAYGTVPVVHAV | 618 | |
| | | Sbjct: | 69 | LGLEDNVI-FLGFVPDEDLPELYKS---ADVFVLPSRYEGFGIVLLEAMACGLPVIATDV | 124 | |
| | | Query: | 619 | GGLRDTVAP------FDPFNDTGLGWTFDRAEAN 646 | | |
| | | Sbjct: | 125 | GGIPEIVKDGETGLLVEPGDVEALAEAIEKLLKD 158 | | |
| DuI | Seq. Id. | Query: | 1478 | PVVGIVTRLTAQKGIHLIKHAIHRTLERNGQVVLLGSAPDSRIQADFVNLANTLHGVNHG | 1537 | 70.3 |
| | No.19 (query) | | | | | |
| | Seq. Id. | Sbjct: | 16 | KIILFVGRLLPEKGIDLLIEAFKKLKKQLNPNLKLVIVGDGEEEDELKLLALKLGLEDNV | 75 | |
| | No.20 (snjct) | | | | | |
| | | Query: | 1538 | QVRLSLTYDEPLSHLIYAGSDFILVPSIFEPCGLTQLVAMRYGTIPIVRKTGGLFDTVFD | 1597 | |
| | | Sbjct: | 76 | IFLGFVPDEDLPEL--YKSADVFVLPSRYEGFGIVLLEAMACGLPVIATDVGGIPEIVKD | 133 | |
| | | Query: | 1598 | VDN 1600 | | |
| | | Sbjct: | 134 | GET 136 | | |

TABLE IIIa
PIR Multiple Alignment for “GLYTR” Domain of Maize SS enzymes

| Enzyme | Seq | |
|---|---|---|
| (res. # of | Id | |
| the start) | No | CLUSTAL W (1.8) multiple sequence alignment |
| SSIIa-540 | 21 | -----------EVRDDVPLLGFIGRLDGQKGVDIIGDAMPWIAG--QDVQLVMLGTG-----RADLERMLQHLEREHPNKVRGWVGFS |
| | | VPMAHRITAGADVLVMPSFEPCGLNQLYAMAYGTVPVVHAVGGLRDTVAPFDP-FGDAGLGWTFDRAEANKLIEALR---- |
| SSIIb-506 | 22 | ----------LQVRDDVPLIGFIGRLDHQKGVDIIADAIHWIAG--QDVQLVMLGTG-----RADLEDMLRRFESEHSDKVRAWVGFS |
| | | VPLAHRITAGADILLMPSRFEPCGLNQLYAMAYGTVPVVHAVGGLRDTAPFDP-FNDTGLGWTFDRAEAN----------- |
| SSI-441 | 23 | ----------LPIRPDVPLIGFIGRLDYQKGIDLIQLIIPDLMR--EDVQFVMLGSG-----DPELEDWMRSTESIKDKFRGWVGFSV |
| | | PVSHRITAGCDILLMPSRFEPCGLNQLYAMQYGTVPVVHATGGLRDTVENFNP-FGENG---------------------- |
| GBSS-378 | 24 | NKEALQAEVGLPVDRNIPLVAFIGRLEEQKGPDVMAAAIPQLMEMVEDVQIVLLGTG-----KKKFERMLMSAEEKFPGKVRAVVKFN |
| | | AALANHIMAGADVLAVTSRFEPCGLIQLQGMRYGTPCACASTGGLVDTIIEGKTGFEMGRLSVDCNVVEPADVKKVATTLC |
| Dul-1478 | 25 | -----------------PVVGIVTRLTAQKGIHLIKHAIHRTLE--RNGQVVLLGSAPDS RIQADFVNLANTHGVNHGQVRLSLTYD |
| | | *::.::** *** .:: : .: *.*:**:. .: . .:.* :. . |
| | | EPLSHLIYAGSDFILVPSIFEPCGTQLVAMR YGTIPIVRKTGGLFDTVFDVDN--------------------------- |
| | | ::* * **.*.: :.* ****** ** .* *** .*** **: . |

Footnote for all the sequence alignments and comparisons in this document:
*= identical amino acid
:.= similar amino acids
-= gaps for accommodating residues from other sequences that are not aligned.

TABLE IIIb
PIR Multiple Alignment of SSI and SSII enzymes

| | | SEQ.ID. |
|---|---|---|
| ENZYME | CLUSTAL W (1.8) multiple sequence alignment | No |
| SSIIA. | MSSAAVSSSSSTFFLALASASPGGRRRARVGSSP---FHTGASLSFAFWAPPSPPRAPRD | 26 |
| SSIIB. | -MPGAISSSSSAFLLPVASSSPR-RRRGSVGAALRSYGYSGAELR-LHWARRGPPQD--G | 27 |
| SSI. | --MATPSAVGAACLLLARAAWP-----AAVG-----------DR-----ARPRRLQR--- | 29 |
| | .: *: .:: :* :: * . ** . * : | |
| SSIIA. | AALVRAEAEAGGKDAPPERSGDAARLPRARRNAVSKRRDPLQPVGRYGSATGNTARTGAA | 26 (cont.d) |
| SSIIB. | AASVRAAAAPAGGESEEAAKSSSSSQAGAVQGSTAKAVDSASPPNPLTSAP---KQSQSA | 27 |
| SSI. | -VLRRRCVAELSREGPAPRPLPPALLA------P-----PLVP--------------------- | 29 |
| | . * . . :. .: . . * | |
| SSIIA. | SCQNAALADVEIKSIVAAPPTSIVKFPAPGYRMILPSGDIAPETVLPAPKPLHESPAVDG | 26 (cont.d) |
| SSIB. | AMQNG------------------------------TSGGSSASTAAPVSGPKADHPSAPV | 27 |
| SSI. | ---------------------------------------G-FLAPPAEPTGEPASTPPPVP- | 29 |
| | * . .. *. * *.. | |
| SSIIA. | DSNGIAPPTVEPLVQEATWDFKKYIGFDEPDEAKDDSRVGADDAGSFEHYG-DNDSGPLA | 26 (cont.d) |
| SSIIB. | TKREIDASAVKPEPAGDDARPVESIGIAEPVDAKADAAPATDAAASAPYDREDNEPGPLA | 27 |
| SSI. | -DAGLGDLGLEPE------------GIAEG--SIDNTVVVASEQDSEIVVGKEQARAKVT | 29 |
| | . : ::* *: * : :: :. * :: . :: | |
| SSIIA. | GENVMNVIVVAAECSPWCKTGGLGDVVGALPKALARRGHRVMVVVPRYG------DYVEA | 26 (cont.d) |
| SSIIB. | GPNVMNVVVVASECAPFCKTGGLGDVVGALPKALARRGHRVMVVIPRYG------EYAEA | 27 |
| SSI. | ----QSIVFVTGEASPYAKSGGLGDVCGSLPVALAARGHRVMVVMPRYLNGTSDKNYANA | 29 |
| | .::.*:.*.:*:.*:****** *:** *** ********:*** :*.:* | |
| SSIIA. | FDMGIRKYYKAAGQDLEVNYFHAFIDGVDFVFIDAPLFRHRQDDIYG---GSRQEIMKRM | |
| SSIIB. | RDLGVRRRYKVAGQDSEVTYFHSYIDGVDFVFVEAPPFRHRHNNIYG---GERLDILKRM | |
| SSI. | FYTEKHIRIPCFGGEHEVTFFHEYRDSVDWVFVDHPSY-HRPGNLYGDKFGAFGDNQFRY | |
| | : * : **.:** : *.**:**:: * : ** .::** * : * | |
| SSIIA. | ILFCKVAVEVPWHVPCGGVCYGDGNLVFIANDWHTALLPVYLKAYYRDHGLMQYTRSVLV | 26 (cont.d) |
| SSIIB. | ILFCKAAVEVPWYAPCGGTVYGDGNLVFIANDWHTALLPVYLKAYYRDNGLMQYARSVLV | 27 |
| SSI. | TLLCYAACEAPLILELGGYIYG-QNCMFVVNDWHASLVPVLLAAKYRPYGVYKDSRSILV | 29 |
| | *:* .* *.* ** ** * :*:.****::*:** * * ** *: : :**:** | |
| SSIIA. | IHNIAHQGRGPVDEFPYMDLP------EHYLQHFELYDPVG-GEHANIFAAGLKMADRV | 26 (cont.d) |
| SSIIB. | IHNIAHQGRGPVDDFVNFDLP------EHYIDHFKLYDNIG-GDHSNVFAAGLKTADRV | 27 |
| SSI. | IHNLAHQGVEPASTYPDLGLPPEWYGALEWVFPEWARRHALDKGEAVNFLKGAVVTADRI | 29 |
| | ***:**** *.. : :.** * : .: . :. *: *.: ..: ***: | |
| SSIIA. | VTVSRGYLWELKTVEGGWGLHDIIRSNDWKINGIVNGIDHQEWNPKVDVHLRSDGYTNYS | 26 (cont.d) |
| SSIIB. | VTVSNGYMWELKTSEGGWGLHDIINQNDWKLQGIVNGIDMSEWNPAVDVHLHSDDYTNYT | 27 |
| SSI. | VTVSKGYSWEVTTAEGGQGLNELLSSRKSVLNGIVNGIDINDWNPATDKCIP----CHYS | 29 |
| | ****.** **:.* *** **:::: ... ::******* .:*** .* : :*: | |
| SIIA. | LETLDAGKRQCKAALQRELGLEVRDDVPLLGFIGRLDGQKGVDIIGDAMPWIAGQDVQLV | 26 (cont.d) |
| SSIIB. | FETLDTGKRQCKAALQRQLGLQVRDDVPLIGFIGRLDHQKGVDIIADAIHWIAGQDVQLV | 27 |
| SSI. | VDDLS-GKAKCKGALQKELGLPIRPDVPLIGFIGRLDYQKGIDLIQLIIPDLMREDVQFV | 29 |
| | : *. ** :**.***::*** :* ****:******* ***:*:* : : :***:* | |
| | MLGTGRADLERMLQHLEREHPNKVRGWVGFSVPMAHRITAGADVLVMPSRFEPCGLNQLY | 26 (cont.d) |
| SSIIA. | MLGTGRADLEDMLRRFESEHSDKVRAWVGFSVPLAHRITAGADILLMPSRFEPCGLNQLY | 27 |
| SSIIB. | MLGSGDPELEDWMRSTESIFKDKFRGWVGFSVPVSHRITAGCDILLMPSRFEPCGLNQLY | 29 |
| SSI. | ***:* .:** :: * . *.*. *******::******.*:*:************** | |
| SSIIA. | AMAYGTVPVVHAVGGLRDTVAPFDPFGDAG---LGWTFDRAEANKLIEALRHCLDTYRKY | 26 (cont.d) |
| SSIIB. | AMAYGTVPVVHAVGGLRDTVAPFDPFNDTG---LGWTFDRAEANRMIDALSHCLTTYRNY | 27 |
| SSI. | AMQYGTVPVVHATGGLRDTVENFNPFGENGEQGTGWAFAPLTTENMFVDIANCN------ | 29 |
| | ** *********.******* *:**.: * ::.:: : :* | |
| SSIIA. | GESWKSLQARGMSQDLSWDHAAELYEDVLVKAKYQW | 26 (cont.d) |
| SSIIB. | KESWRACRARGMAEDLSWDHAAVLYEDVLVKAKYQW | 27 |
| SSI. | ------IYIQGTQVLLGRANEARHVKRLHVGPCR-- | 29 |

TABLE IV
Possible, but not limited to Amino acid Sequences
for Some of the Proposed Fusion Proteins

A. GBSS (61-300) + Du1 (1201-1674)

SEQ.ID. No. 30

RRGGRFPSLV VCASAGMNVV FVGAEMAPWS KTGGLGDVLG GLPPAMAANG HRVMVVSPRY DQYKDAWDTS
VVSEIKMGDG YETVRFFRCY KRGVDRVFVD HPLFLERVWG KTEEKIYGPV AGTDYRDNQL RFSLLCQAAI
EAPRILSLNN NPYFSGPYGE DVVFVCNDWH TGPLSCYLKS NYQSHGIYRD AKTAFCIHNI SYQGRFAFSD
YPELNLPERF KSSFDFIDGY EKPVEGRKIN WMKAGILEAD RVLTVSPYYA EELISGIARG CELDNIMRLT
GITGIVNGMD VSEWDPSRDK GGIYDNRNGL DYHIPVFGSI AKEPPMHIVH IAVEMAPIAK VGGLGDVVTS
LSRAVQDLGH NVEVILPKYG CLNLSNVKNL QIHQSFSWGG SEINVWRGLV EGLCVYFLEP QNGMFGVGYV
YGRDDDRRFG FFCRSALEFL LQSGSSPNII HCHDWSSAPv AWLUKENYAK SSLANARVVF TIHNLEFGAH
HIGKAMRYCD KATTVSNTYS KEVSGHGAIV PHLGKFYGIL NGIDPDIWDP YNDNFIPVHY TCENVVEGKR
AAKRALQQKF GLQQIDVPVV GIVTRLTAQK GIHLIKHAIH RTLERNGQVV LLGSAPDSRI QADFVNLANT
LHGVNHGQVR LSLTYDEPLS HLIYAGSDFI LVPSIFEPCG LTQLVAMRYG TIPIVRKTGG LFDTVFDVDN
DKERARDRGL EPNGFSFDGA DSNGVDYALN RAISAWFDAR SWFHSLCKRV MEQDWSWNRP ALDYIELYRS
ASKL
B. GBSS (61-300) + SSI (400-622)

SEQ.ID. No. 31

RRGGRFPSLV VCASAGMNVV FVGAEMAPWS KTGGLGDVLG GLPPAMAANG HRVMVVSPRY DQYKDAWDTS
VVSEIKMGDG YETVRFFECY KRGVDRVFVD HPLFLERVWG KTEEKIYGPV AGTDYRDNQL RFSLLCQAAL
EAPRILSLNN NPYFSGPYGE DVVFVCNDWH TGPLSCYLKS NYQSHGIYRD AKTAFCIHNI SYQGRFAFSD
YPELNLPERF KSSFDFIDGY EKPVEGRKIN KATTVSNTYS KEVSGHGAIV PHLGKFYGIL NGIDPDIWDP
NGIDINDWNP ATDKCIPCHY SVDDLSGKAK CKGALQKELG LPIRPDVPLI GFIGRLDYQK GIDLIQLIIP
DLMREDVQFV MLGSGDPELE DWMRSTESIF KDKFRGWVGF SVPVSHRITA GCDILLMPSR FEPCGLNQLY
AMQYGTVPVV HATGGLRDTV ENFNPFGENG EQGTGWAFAp LTTENMFVDI ANCNIYIQGT QVLLGRANEA
RHVKRLHVGP CR
C. GBSS (61-300) + SSIIa (481-732)

SEQ.ID. No. 32

RRGGRFPSLV VCASAGMNVV FVGAEMAPWS KTGGLGDVLG GLPPAMAANG HRVMVVSPRY DQYKDAWDTs
VVSEIKMGDG YETVRFFHCY KRGVDRVFVD HPLFLERVWG KTEEKIYGPV AGTDYRDNQL RFSLLCQAAL
EAPRILSLNN NPYFSGPYGE DVVFVCNDWH TGPLSCYLKS NYQSHGIYRD AKTAFCIHNI SYQGRFAFSD
YPELNLPERF KSSFDFIDGY EKPVEGRKIN DIIRSNDWKI NGIVNGIDHQ EWNPKVDVHL RSDGYTNYSL
ETLDAGKRQC KAALQRELGL EVRDDVPLLG FIGRLDGQKG VDIIGDAMPW IAGQDVQLVM LGTGRADLER
MLQHLEREHP NKVRGWVGFS VPMAHRITAG ADVLVMPSRF EPCGLNQLYA MAYGTVPVVH AVGGLRDTVA
PFDPFGDAGL GWTFDRAEAN KLIEALRUCL DTYRKYGESW KSLQARGMSQ DLSWDHAAEL YEDVLVKAKY
QW
D. GBSS (61-300) + SSIIb (481-698)

SEQ.ID. No. 33

RRGGRFPSLV VCASAGMNVV FVGAEMAPWS KTGGLGDVLG GLPPAMAANG HRVMVVSPRY DQYKDAWDTS
VVSEIKMGDG YETVRFFHCY KRGVDRVFVD HPLFLERVWG KTEEKIYGPV AGTDYRDNQL RFSLLCQAAL
EAPRILSLNN NPYFSGPYGE DVVFVCNDWH TGPLSCYLKS NYQSHGIYRD AKTAFCIHNI SYQGRFAFSD
YPELNLPERF KSSFDFIDGY EKPVEGRKIN YTNYTFETLD TGKRQCKAAL QRQLGLQVPD DVPLIGFIGR
LDHQKGVDII ADAIHWIAGQ DVQLVMLGTG RADLEDMLRR FESEHSDKVR AWVGFSVPLA HRITAGADIL
LMPSRFEPCG LNQLYAMAYG TVPVVHAVGG LRDTVAPFDP FNDTGLGWTF DRAEANRMID ALSHCLTTYR
NYKESWRACR ARGMAEDLSW DHAAVLYEDV LVKAKYQW

Amino acid sequence
of maize granule bound starch synthase (GBSS)

```
Accession numbers: EMBL; X03935; E276624; -;
                                      PIR; S07314; S07314;
                                      MAIZEDB; 15806
```

SEQ.ID. No. 34

| | |
|---|---|
| 1 | M A A L A T S Q L V A T R A G L G V P D A S T F R R G A A Q |
| 31 | G L R G A R A S A A A D T L S M R T S A R A A P R H Q Q Q A |
| 61 | R R G G R F P S L V V C A S A G M N V V F V G A E M A P W S |
| 91 | K T G G L G D V L G G L P P A M A A N G H R V M V V S P R Y |
| 121 | D Q Y K D A W D T S V V S E I K M G D G Y E T V R F F H C Y |
| 151 | K R G V D R V F V D H P L F L E R V W G K T E E K I Y G P V |
| 181 | A G T D Y R D N Q L R F S L L C Q A A L E A P R I L S L N N |
| 211 | N P Y F S G P Y G E D V V F V C N D W H T G P L S C Y L K S |
| 241 | N Y Q S H G I Y R D A K T A F C I H N I S Y Q G R F A F S D |
| 271 | Y P E L N L P E R F K S S F D F I D G Y E K P V E G R K I N |
| 301 | W M K A G I L E A D R V L T V S P Y Y A E E L I S G I A R G |
| 331 | C E L D N I M R L T G I T G I V N G M D V S E W D P S R D K |
| 361 | Y I A V K Y D V S T A V E A K A L N K E A L Q A E V G L P V |
| 391 | D R N I P L V A F I G R L E E Q K G P D V M A A A I P Q L M |
| 421 | E M V E D V Q I V L L G T G K K K F E R M L M S A E E K F P |
| 451 | G K V R A V V K F N A A L A H H I M A G A D V L A V T S R F |
| 481 | E P C G L I Q L Q G M R Y G T P C A C A S T G G L V D T I I |
| 511 | E G K T G F H M G R L S V D C N V V E P A D V K K V A T T L |
| 541 | Q R A I K V V G T P A Y E E M V R N C M I Q D L S W K G P A |
| 571 | K N W E N V L L S L G V A G G E P G V E G E E I A P L A K E |
| 601 | N V A A P |

TABLE VII
Maize Granule bound starch synthase (GBSS)
Aligmnents with other similar proteins-Transit Peptide

| SEQ | Accession | a.a | | a.a. |
|---|---|---|---|---|
| Id.No. | Number | # | Sequence | # |
| 35 | maize GBSS | 1 | MAALATSQLVATRAGLGVPD----ASTF--XXXXXXXXXXXXXXXXXDTLSMRT---S-- | 49 |
| 36 | 136757 | 1 | MAALAT8QLVATRAGLGVPD----ASTF--RRGAAQGLRGARASAAADTLSMRT---S-- | 49 |
| 37 | 2833385 | 1 | MSTLATSQLVATHAGLGVPD----ASMFRRGGVQGLRAAARASAAAGDALSMRT---SAC | 53 |
| 38 | 136758 | 1 | MSALTTSQLATSATGFGIADRSAPSSLL--RHGFQGLKPRSPAGGDATSLSVTT---S-- | 53 |
| 39 | 2833382 | 1 | MSALTTSQLATSATGFGIADRSAPSSLL--REGFQGLKPRSPAGGDATSLSVTT---S-- | 53 |
| 40 | 297424 | 1 | MSALTTSQLATSATGFGIADRSAPSSLL--RHGFQGLKPRSPAGGDATSLSVTT---D-- | 53 |
| 41 | 7798551 | 1 | MSALTTSQLATSATGFGIADRSAPSSLL--RHGFQGLKPRSPAGGDATSLSVTT---S-- | 53 |
| 42 | 82478 | 1 | MSALTTSQLATSATGFGIADRSAPSSLL--RHGFQGLKPRSPAGGDATSLSVTT---S-- | 53 |
| 43 | 297422 | 1 | MSALTTSQLATSATGFGIADRSAPSSLL--RHGFQGLKPRSPAGGDASSLSVTT---S-- | 53 |
| 44 | 136755 | 1 | MAALATSQLATSGTVLGVTD----------RFRRPGFQGLRPRNPADAALGMRT---I-- | 45 |
| 45 | 18652407 | 1 | MAALATSQLATSGTVLGVTD----------RFRRPGFQGLRPRNPADAALGMRT---I-- | 45 |
| 46 | 4760582 | 1 | MAALVTSQLATSGTVLGITD----------RFRRAGFQGVRPRSPADAPLGMRTTGAS-- | 48 |
| 47 | 11037536 | 1 | MAALVTSQLATSGTVLGITD----------RFRRAGFQGVRPRSPADAPLGMRTTGAS-- | 48 |
| 48 | 6624287 | 1 | MAALVTSQLATSGTVLGITD----------RFRRAGFQGVRPRNPADAALVMRT---I-- | 45 |
| 49 | 6624283 | 1 | MAALVTSQLATSGTVLGITD----------RFRRAGFQGVRPRSPADAPLGMRTTGAS-- | 48 |
| 50 | 4760584 | 1 | MAALVTSQLATSGTVLGITD----------RFRRAGFQGVRPRSPADAALGMRT---V-- | 45 |
| 51 | 6318538 | 1 | MAALVTSQLATSGAVLGITD----------RFRRAGFQGVRPRSPADAPLGMRTVGAS-- | 48 |
| 52 | 6318540 | 1 | MAALVTSQLATSATVLGITD----------RFRRAGFQGVRPRSPADAPLGMRTVGAS-- | 48 |
| 53 | 6624285 | 1 | MAALVTSQLATSGTVLSVTD----------RFRRPGFQGLRPRNPADAALGMRT---V-- | 45 |
| 54 | 6624281 | 1 | MAALVTSQLATSGTVLSVTD----------RFRRPGFQGLRPRNPADAALGMRTVG-A-- | 47 |
| 55 | 4760580 | 1 | MAALVTSQLATSGTVLSVTD----------RFRRPGFQGLRPRNPADAALGMRTVG-A-- | 47 |
| 56 | 136765 | 1 | MAALVTSQLATSGTVLSVTD----------RFRRPGFQGLRPRNPADAALGMRTVG-A-- | 47 |
| 57 | 4588609 | 1 | MAALVTSQLATSGTVLGITD----------RFRRAGFQGVRPRSPADAALGMRT---V-- | 45 |
| 58 | 6136121 | 49 | DKLQMRN---N-- | 56 |
| 59 | 15637079 | 48 | DMLQLRT---S-- | 55 |
| 60 | 2833388 | 50 | DKLQMKT---Q-- | 57 |
| 35 (cont.d) | GBSS | 50 | -XXXXXXXXXXXXXGG--RFPSLVVCA--S-AGMNVVFVGAEMAPWSKTXXXXXXXXXXP | 103 |
| 36 (cont.d) | 136757 | 50 | -ARAAPRHQQQARRCG--RFPSLVVCA--S-AGMNVVFVGAEMAPWSKTGGLGDVLGGLP | 103 |
| 37 (cont.d) | 2833385 | 54 | PAPRQQPAARRGGRGG--RFPSLVVCA--T-AGMNVVFVGAEMAPWSKTGGLGDVLGGLP | 108 |
| 38 (cont.d) | 136758 | 54 | -APATPKQQRSVQRGSR-RFPSVVVYA--TGAGMNVVFVGAEMAPWSKTGGLGDVLGGLP | 109 |
| 39 (cont d) | 2833382 | 54 | -ARATPKQQRSVQRGSR-RFPSVVVYA--TGAGNNVVFVGAEMAPWSKTGGLGDVLGGLP | 109 |
| 40 (cont.d) | 297424 | 54 | -ARATPKQQRSVQRGSR-RFPSVVVYA--TGAGNNVVFVGAEMAPWSKTGGLGDVLGGLP | 109 |
| 41 (cont.d) | 7798551 | 54 | -APATPKQQRSVQRGSR-RFPSVVVYA--TGAGNNVVFVGAEMAPWSKTGGLGDVLGGLP | 109 |
| 42 (cont.d) | 82478 | 54 | -ARATPKQQRSVQRGSR-RFPSVVVYA--TGAGMNVVFVGAEMAPWSKTGGLGDVLGGLP | 109 |
| 43 (cont.d) | 297422 | 54 | -ARATPKQQRSVQRGSR-RFPSVVVYA--TGAGMNVVFVGAEMAPWSKTGGLGDVLGGLP | 109 |
| 44 (cont d) | 136755 | 46 | -GASAAPKQSRKAHRGSRRCLSVVVRA--TGSGMNLVFVGAEMAPWSKTGGLGDVLGGLP | 102 |
| 45 (cont.d) | 18652407 | 46 | -GASAAPKQSRKAHRGSRRCLSVVVRA--TGSGMNLVFVGAEMAPWSKTGGLGDVLGGLP | 102 |
| 46 (cont.d) | 4760582 | 49 | -AAPKQQSRKAHRGTR--RCLSMVVRATGS-AGMNLVFVGAEMAPWSKTGGLGDVLGGLP | 104 |
| 47 (cont.d) | 11037536 | 49 | -AAPKQQSRKAHRGTR--RCLSMVVRATGS-AGMNLVFVGAEMAPWSKTGGLGDVLGGLP | 104 |
| 48 (cont.d) | 6624287 | 46 | -GASAAPKQSRKAHRGSRRCLSMVVRATGS-GGMNLVFVGAEMAPWSKTGGLGDVLGGLP | 103 |
| 49 (cont.d) | 6624283 | 49 | -AAPKQQSRKAHRGTR--RCLSMVVRATGS-AGMNLVFVGAEMAPWSKTGGLGDVLGGLP | 104 |
| 50 (cont.d) | 4760584 | 46 | -GASAAPTQSRKAHRGTRRCLSMVVRATGS-GGMNLVFVGAEMAPWSKTGGLGDVLGGLP | 103 |
| 51 (cont.d) | 6318538 | 49 | -AAPKQQSRKAHRGTR--RCLSVVVRATGS-GGMNLVFVGAEMAPWSKTGGLGDVLGGLP | 104 |
| 52 (cont.d) | 6318540 | 49 | -AAPKQQSRKAHRGTR--RCLSMVVRATGS-GGMNLVFVGAEMAPWSKTGGLGDVLGGLP | 104 |
| 53 (cont.d) | 17736918 | 46 | --S-GGMNLVFVGAEMAPWSKTGGLGDVLGGLP | 33 |
| 54 (cont.d) | 6624285 | 46 | -GASAAPKQSRKPHRGNRRCLSMVVRATGS-GGMNLVFVCAEMAPW5KTGGLGDVLGGLP | 103 |
| 55 (cont.d) | 6624281 | 48 | -SAAPKQSRKPHRFDR--RCLSMVVRATGS-GGMNLVFVGAEMAPWSKTGGLGDVLGGLP | 103 |
| 56 (cont.d) | 4760580 | 48 | -SAAPKQSRKPHRFDR-RCLSMVVR ATGS-GGMNLVFVGAEMAPWSKTGGLGDVLGGLP | 103 |
| 59 (cont.d) | 136765 | 48 | -SAAPKQSRKPHRFDR--RCLSMVVRATGS-GGMNLVFVGAEMAPWSKTGGLGDVLGGLP | 103 |
| 57 (cont.d) | 4588609 | 46 | -GASAAPTQSRKAHRGTRRCLSMVVRATGS-GGMNLVFVGAEMAPWSKTGGLGDVLGGLP | 103 |
| 62 (cont.d) | 4588607 | 36 | A--D-AALGIRTVGASAAP--KQSRKPHRGNRRC | 64 |
| 58 (cont.d) | 6136121 | 57 | -AKQSRSLVKKTDNGS--PLGK-IICG--T--GMNLVFVLAEVGPWSKTGGLGDVVGGLP | 108 |
| 59 (cont.d) | 15637079 | 56 | --AKKPSKNGRENEGG--MAAGTIVCK--Q-QGMNLVFVGCEVGPWCKTGGLGDVLGGLP | 108 |
| 63 (cont.d) | 3832512 | 75 | IVCG--N--GMNLVFVGAEVGPWSKTGGLGDVLGGLP | 107 |
| 64 (cont.d) | 2833381 | 75 | IVCK--Q-QGMNLVFVGCEEGPWCKTGGLGDVLGGLP | 108 |
| 65 (cont.d) | 12003285 | 73 | CE--T-SGMTLIFVSAECGPWSKTGGLGDVVGGLP | 104 |
| 66 (cont.d) | 228210 | 75 | IVCG--K--GMNLIFVGTEVGPWSKTGGLGDVLGGLP | 107 |
| 67 (cont.d) | 15626365 | 82 | IIVC-----GMNLIFVGTEVAPWSKTGGLGDVLGGLP | 113 |
| 68 (cont.d) | 267196 | 75 | IVCG--K--GMNLIFVGTEVGPWSKTGGLGDVLGGLP | 107 |
| 60 (cont.d) | 2833388 | 58 | ---SKAVKKVSATGNG--RPAAKIICG--H--GMNLIFVGAEVGPWSKTGGLGDVLGGLP | 108 |
| 69 (cont.d) | 602594 | 75 | IVCG--K--GMNLIFVGTEVGPWSKTGGLGDVLGGLP | 107 |
| 70 (cont.d) | 15223331 | 78 | IVC---E-KGMSVIFIGAEVGPWSKTGGLGDVLGGLP | 110 |
| 71 (cont.d) | 5441242 | 79 | GMNLIFVGAEVAPWSKTGGLGDVLGGLP | 106 |
| 72 (cont.d) | 18139611 | 75 | IVC---S-AGMTIIFIATECHPWCKTGGLGDVLGGLP | 107 |
| 73 (cont.d) | 2833383 | 73 | IVC------GMSLVFVGAEVGPWSKTGGLGDVLGGLP | 103 |
| 74 (cont.d) | 6492245 | 61 | --RTPAPIVC---S-TGMPIIFVATEVHPWCKTGGLGDVVGGLP | 98 |

TABLE VIII
Maize (GBSS) “GLASS” Domain and it's Alignments with other similar proteins

| SEQ | Accession | a.a | | a.a. |
|---|---|---|---|---|
| Id.No. | Number | # | (start) “GLASS” Sequence (ending) | # |
| 75 | MAIZE-GESS | 104 | PAMAANGHRVMVVSPRYDQYKDAWDTSVVSEIKMGDGYETVRFFHCYKRGVDRVFVDHPL | 163 |
| 76 | 136757 | 104 | PAMAANGHRVMVVSPRYDQYKDAWDTSVVSEIKMGDGYETVRFFHCYKRGVDRVFVDHPL | 163 |
| 77 | 2833385 | 109 | PAMAANGHRVMVVSPRYDQYKDAWDTSVVSEIKMGDGYETVRFFHCYKRGVDRVFVDHPL | 168 |
| 78 | 136758 | 110 | PAMAANGHRVMVVSPRYDQYKDAWDTSVVSEIKMGDGYETVRFFHCYKRGVDRVFVDHPL | 169 |
| 79 | 2833382 | 110 | PAMAANGHRVMVVSPRYDQYKDAWDTSVVSEIKMGDGYETVRFFHCYKRGVDRVFVDHPL | 169 |
| 80 | 297424 | 110 | PAMAANGHRVMVVSPRYDQYKDAWDTSVVSEIKMGDGYETVRFFHCYKRGVDRVFVDHPL | 169 |
| 81 | 7798551 | 110 | PAMAANGHRVMVVSPRYDQYKDAWDTSVVSEIKMGDGYETVRFFHCYKRGVDRVFVDHPL | 169 |
| 82 | 82478 | 110 | PAMAANGHRVMVVSPRYDQYKDAWDTSVVSEIKMGDGYETVRFFHCYKRGVDRVFVDHPL | 169 |
| 83 | 297422 | 110 | PAMAANGHRVMVVSPRYDQYKDAWDTSVVSEIKMGDGYETVRFFHCYKRGVDRVFVDHPL | 169 |
| 84 | 136755 | 103 | PAMAANGHRVMVVSPRYDQYKDAWDTSVVSEIKMGDGYETVRFFHCYKRGVDRVFVDHPL | 162 |
| 85 | 18652407 | 103 | PAMAANGHRVMVVSPRYDQYKDAWDTSVVSEIKMGDGYETVRFFHCYKRGVDRVFVDHPL | 162 |
| 86 | 4760582 | 105 | PAMAANGHRVMVVSPRYDQYKDAWDTSVVSEIKMGDGYETVRFFHCYKRGVDRVFVDHPL | 164 |
| 87 | 11037536 | 105 | PAMAANGHRVMVVSPRYDQYKDAWDTSVVSEIKMGDGYETVRFFHCYKRGVDRVFVDHPL | 164 |
| 88 | 6624287 | 104 | PAMAANGHRVMVVSPRYDQYKDAWDTSVVSEIKMGDGYETVRFFHCYKRGVDRVFVDHPL | 163 |
| 89 | 6624283 | 105 | PAMAANGHRVMVVSPRYDQYKDAWDTSVVSEIKMGDGYETVRFFHCYKRGVDRVFVDHPL | 164 |
| 90 | 4760584 | 104 | PAMAANGHRVMVVSPRYDQYKDAWDTSVVSEIKMGDGYETVRFFHCYKRGVDRVFVDHPL | 163 |
| 91 | 6318538 | 105 | PAMAANGRRVMVISPRYDQYKDAWDTSVVSEIKVADEYERVRYFHCFKRGVDRVFVDHPC | 164 |
| 92 | 6318540 | 105 | PAMAANGHRVMVVSPRYDQYKDAWDTSVVSEIKMGDGYETVRFFHCYKRGVDRVFVDHPL | 164 |
| 93 | 17736918 | 34 | PAMAANGHRVMVVSPRYDQYKDAWDTSVVSEIKMGDGYETVRFFHCYKRGVDRVFVDHPL | 93 |
| 94 | 6624285 | 104 | AAMAANGHRVMVISPRYDQYKDAWDTSVISEIKVVDRYERVRYFHCYKRGVDRVFVDHPC | 163 |
| 95 | 6624281 | 104 | AAMAANGHRVMVISPRYDQYKDAWDTSVISEIKVVDRYERVRYFHCYKRGVDRVFVDHPC | 163 |
| 96 | 4760580 | 104 | AAMAANGHRVMVISPRYDQYKDAWDTSVISEIKVVDRYERVRYFHCYKRGVDRVFVDHPC | 163 |
| 97 | 136765 | 104 | AAMAANGHRVMVISPRYDQYKDAWDTSVISEIKVVDRYERVRYFHCYKRGVDRVFVDHPC | 163 |
| 98 | 4588609 | 104 | PAMAANGHRVMVVSPRYDQYKDAWDTSVVSEIKMGDGYETVRFFHCYKRGVDRVFVDHPL | 163 |
| 99 | 4588607 | 65 | LSMVANGHRVMVISPRYDQYKDAWDTSVISEIKVVDRYERVRYFHCYKRGVDRVFVDHPC | 124 |
| 100 | 6136121 | 109 | PAMAANGHRVMVVSPRYDQYKDAWDTSVVSEIKMGDGYETVRFFHCYKRGVDRVFVDHPL | 168 |
| 101 | 15637079 | 109 | PALAARGHRVMTVCPRYDQYKDAWDTCVWELQVGDRIEPVRFFHSYKRGVDRVFVDHPM | 168 |
| 102 | 3832512 | 108 | PALAARGHRVMTVCPRYDQYKDAWDTCVWELQVGDRIEPVRFFHSYKRGVDRVFVDHPM | 167 |
| 103 | 2833381 | 109 | PALAARGHRVMTVCPRYDQYKDAWDTCVWELQVGDRIEPVRFFHSYKRGVDRVFVDHPM | 168 |
| 104 | 12003285 | 105 | PALAARGHRVMTVCPRYDQYKDAWDTCVWELQVGDRIEPVRFFHSYKRGVDRVFVDHPM | 164 |
| 105 | 228210 | 108 | PALAARGHRVMTVCPRYDQYKDAWDTCVWELQVGDRIEPVRFFHSYKRGVDRVFVDHPM | 167 |
| 106 | 15626365 | 114 | PALAARGHRVMTVCPRYDQYKDAWDTCVWELQVGDRIEPVRFFHSYKRGVDRVFVDHPM | 173 |
| 107 | 267196 | 108 | PALAARGHRVMTVCPRYDQYKDAWDTCVWELQVGDRIEPVRFFHSYKRGVDRVFVDHPM | 167 |
| 108 | 2833388 | 109 | PAMAANGHRVMVVSPRYDQYKDAWDTSVVSEIKMGDGYETVRFFHCYKRGVDRVFVDHPL | 168 |
| 109 | 602594 | 108 | PALAARGHRVMTVCPRYDQYKDAWDTCVWELQVGDRIEPVRFFHSYKRGVDRVFVDHPM | 167 |
| 110 | 15223331 | 111 | PALAARGHRVMTVCPRYDQYKDAWDTCVWELQVGDRIEPVRFFHSYKRGVDRVFVDHPM | 170 |
| 111 | 5441242 | 107 | SALAEHGHRVMTVSPRYDQYKDAWDTNVTVEVKVADRIETVRFFHCYKQGVDRVFVDHPC | 166 |
| 112 | 18139611 | 108 | PALAAMGHRVMTIVPRYDQYKDAWDTNVLVEVNIGDRTETVRFFHCYKRGVDRVFVDHPM | 167 |
| 113 | 2833383 | 104 | PVLAGNGHRVMTVSPRYDQYKDAWDTNVLVEVKVGDKIETVRFFHCYKRGVDRVFVDHPL | 163 |
| 114 | 6492245 | 99 | PALAAMGHRVMTIAPRYDQYKDTWDTNVLVEVIVGDRTETVRFFHCYKRGVDRVFVDHPM | 158 |
| 115 | 9587307 | 12 | PAMAANGHRVMTISPRYDQYKDAWDTEVTVELKVGDKTETVRFFHCYKRGVDRVFVDHPM | 71 |
| 116 | 9587337 | 12 | PANAANGHRVMTISPRYDQYKDAWDTEVTVELKVGDKIETVRFFHCYKRGVDRVFVDHPL | 71 |
| 117 | 9587327 | 12 | PAMAANGHRVMTISPRYDQYKDAWDTEVTVELKVGEKIEFVRFFHCYKRGVDRVFVDHPL | 71 |
| 118 | 9587333 | 12 | PANAANGHRVMTVSPRYDQYKDAWDTEVTVELKVGQKIETVRFFHCHKRGVDRVFVDHPL | 71 |
| 119 | 9587329 | 12 | PALAANGHRVMTVSPRYDQYKDAWDTNVLVEIEVGGKIETVRFFHCYKRGVDRVFVDHPL | 71 |
| 120 | 9587325 | 12 | PAMAANGHRVMTISPRYDQYKDAWDTEVTVELNVGEKTETVRFFECYKRGVDRVFVDHPL | 71 |
| 75 | MAIZE-GESS | 164 | FLERVWGKTEEKIYGPVAGTDYRDNQLRFSLLCQAALEAPRILSLNNNPYFSGPY----- | 218 |
| 76 | 136757 | 164 | FLERVWGKTEEKIYGPVAGTDYRDNQLRFSLLCQAALEAPRILSLNNNPYFSGPY----- | 218 |
| 77 | 2833385 | 169 | FLERVWGKTEEKIYGPVAGTDYRDNQLRFSLLCQAALEAPRILSLNNNPYFSGPY----- | 223 |
| 78 | 136758 | 170 | FLERVWGKTEEKIYGPVAGTDYRDNQLRFSLLCQAALEAPRILSLNNNPYFSGPY----- | 224 |
| 79 | 2833382 | 170 | FLERVWGKTEEKIYGPVAGTDYRDNQLRFSLLCQAALEAPRILSLNNNPYFSGPY----- | 224 |
| 80 | 297424 | 170 | FLERVWGKTEEKIYGPVAGTDYRDNQLRFSLLCQAALEAPRILSLNNNPYFSGPY----- | 224 |
| 81 | 7798551 | 170 | FLERVWGKTEEKIYGPVAGTDYRDNQLRFSLLCQAALEAPRILSLNNNPYFSGPY----- | 224 |
| 82 | 82478 | 170 | FLERVWGKTEEKIYGPVAGTDYRDNQLRFSLLCQAALEAPRILSLNNNPYFSGPY----- | 224 |
| 83 | 297422 | 170 | FLERVWGKTEEKIYGPVAGTDYRDNQLRFSLLCQ---EAPRILSLNNNPYFSGPY----- | 221 |
| 84 | 136755 | 163 | FLERVWGKTEEKIYGPVAGTDYRDNQLRFSLLCQAALEAPRILSLNNNPYFSGPY----- | 217 |
| 85 | 18652407 | 163 | FLERVWGKTEEKIYGPVAGTDYRDNQLRFSLLCQAALEAPRILSLNNNPYFSGPY----- | 217 |
| 86 | 4760582 | 165 | FLERVWGKTEEKIYGPVAGTDYRDNQLRFSLLCQAALEAPRILSLNNNPYFSGPY----- | 219 |
| 87 | 11037536 | 165 | FLERVWGKTEEKIYGPVAGTDYRDNQLRFSLLCQAALEAPRILSLNNNPYFSGPY----- | 219 |
| 88 | 6624287 | 164 | FLERVWGKTEEKIYGPVAGTDYRDNQLRFSLLCQAALEAPRILSLNNNPYFSGPY----- | 218 |
| 89 | 6624283 | 165 | FLERVWGKTEEKIYGPVAGTDYRDNQLRFSLLCQAALEAPRILSLNNNPYFSGPY----- | 219 |
| 90 | 4760584 | 164 | FLERVWGKTEEKIYGPVAGTDYRDNQLRFSLLCQAALEAPRILSLNNNPYFSGPY----- | 218 |
| 91 | 6318538 | 165 | FLERVWGKTEEKIYGPVAGTDYRDNQLRFSLLCQAALEAPRILSLNNNPYFSGPY----- | 219 |
| 92 | 6318540 | 165 | FLERVWGKTEEKIYGPVAGTDYRDNQLRFSLLCQAALEAPRILSLNNNPYFSGPY----- | 219 |
| 93 | 17736918 | 94 | FLERVWGKTEEKIYGPVAGTDYRDNQLRFSLLCQAALEAPRILSLNNNPYFSGPY----- | 148 |
| 94 | 6624285 | 164 | FLERVWGKTEEKIYGPVAGTDYRDNQLRFSLLCQAALEAPRILSLNNNPYFSGPY----- | 218 |
| 95 | 6624281 | 164 | FLERVWGKTEEKIYGPVAGTDYRDNQLRFSLLCQAALEAPRILSLNNNPYFSGPY----- | 218 |
| 96 | 4760580 | 164 | FLERVWGKTEEKIYGPVAGTDYRDNQLRFSLLCQAALEAPRILSLNNNPYFSGPY----- | 218 |
| 97 | 136765 | 164 | FLERVWGKTEEKIYGPVAGTDYRDNQLRFSLLCQAALEAPRILSLNNNPYFSGPYAMLCR | 223 |
| 98 | 4588609 | 164 | FLERVWGKTEEKIYGPVAGTDYRDNQLRFSLLCQAALEAPRILSLNNNPYFSGPY----- | 218 |
| 99 | 4588607 | 125 | FLERVWGKTEEKIYGPVAGTDYRDNQLRFSLLCQAALEAPRILSLNNNPYFSGPY----- | 179 |
| 100 | 6136121 | 169 | FLERVWGKTEEKIYGPVAGTDYRDNQLRFSLLCQAALEAPRILSLNNNPYFSGPY----- | 223 |
| 101 | 15637079 | 169 | FLERVWGKTEEKIYGPVAGTDYRDNQLRFSLLCQAALEAPRILSLNNNPYFSGPY----- | 223 |
| 102 | 3832512 | 168 | FLERVWGKTEEKIYGPVAGTDYRDNQLRFSLLCQAALEAPRILSLNNNPYFSGPY----- | 222 |
| 103 | 2833381 | 169 | FLERVWGKTEEKIYGPVAGTDYRDNQLRFSLLCQAALEAPRILSLNNNPYFSGPY----- | 223 |
| 104 | 12003285 | 165 | FLERVWGKTEEKIYGPVAGTDYRDNQLRFSLLCQAALEAPRILSLNNNPYFSGPY----- | 219 |
| 105 | 228210 | 168 | FLERVWGKTEEKIYGPVAGTDYRDNQLRFSLLCQAALEAPRILSLNNNPYFSGPY----- | 222 |
| 106 | 15626365 | 174 | FLERVWGKTEEKIYGPVAGTDYRDNQLRFSLLCQAALEAPRILSLNNNPYFSGPY----- | 228 |
| 107 | 267196 | 168 | FLERVWGKTEEKIYGPVAGTDYRDNQLRFSLLCQAALEAPRILSLNNNPYFSGPY----- | 222 |
| 108 | 2833388 | 169 | FLERVWGKTEEKIYGPVAGTDYRDNQLRFSLLCQAALEAPRILSLNNNPYFSGPY----- | 223 |
| 109 | 602594 | 168 | FLERVWGKTEEKIYGPVAGTDYRDNQLRFSLLCQAALEAPRILSLNNNPYFSGPY----- | 222 |
| 110 | 15223331 | 171 | FLERVWGKTEEKIYGPVAGTDYRDNQLRFSLLCQAALEAPRILSLNNNPYFSGPY----- | 225 |
| 111 | 5441242 | 167 | FLERVWGKTEEKIYGPVAGTDYRDNQLRFSLLCQAALEAPRILSLNNNPYFSGPY----- | 221 |
| 112 | 18139611 | 168 | FLERVWGKTEEKIYGPVAGTDYRDNQLRFSLLCQAALEAPRILSLNNNPYFSGPY----- | 222 |
| 113 | 2833383 | 164 | FLERVWGKTEEKIYGPVAGTDYRDNQLRFSLLCQAALEAPRILSLNNNPYFSGPY----- | 218 |
| 114 | 6492245 | 159 | FLERVWGKTEEKIYGPVAGTDYRDNQLRFSLLCQAALEAPRILSLNNNPYFSGPY----- | 213 |
| 115 | 9587307 | 72 | FLERVWGKTEEKIYGPVAGTDYRDNQLRFSLLCQAALEAPRILSLNNNPYFSGPY----- | 126 |
| 116 | 9587337 | 72 | FLERVWGKTEEKIYGPVAGTDYRDNQLRFSLLCQAALEAPRILSLNNNPYFSGPY----- | 126 |
| 117 | 9587327 | 72 | FLERVWGKTEEKIYGPVAGTDYRDNQLRFSLLCQAALEAPRILSLNNNPYFSGPY----- | 126 |
| 118 | 9587333 | 72 | FLERVWGKTEEKIYGPVAGTDYRDNQLRFSLLCQAALEAPRILSLNNNPYFSGPY----- | 126 |
| 119 | 9587329 | 72 | FLERVWGKTEEKIYGPVAGTDYRDNQLRFSLLCQAALEAPRILSLNNNPYFSGPY----- | 126 |
| 120 | 9587325 | 72 | FLERVWGKTEEKIYGPVAGTDYRDNQLRFSLLCQAALEAPRILSLNNNPYFSGPY----- | 126 |
| 75 | MAIZE-GESS | 219 | ------GEDVVFVCNDWHTGPLSCYLKSNYQSHGIYRDAKTAFCIHNISYQGRFAFSDYP | 272 |
| 76 | 136757 | 219 | ------GEDVVFVCNDWHTGPLSCYLKSNYQSHGIYRDAKTAFCIHNISYQGRFAFSDYP | 272 |
| 77 | 2833385 | 224 | ------GEDVVFVCNDWHTGPLSCYLKSNYQSHGIYRDAKTAFCIHNISYQGRFAFSDYP | 277 |
| 78 | 136758 | 225 | ------GEDVVFVCNDWHTGPLSCYLKSNYQSHGIYRDAKTAFCIHNISYQGRFAFSDYP | 278 |
| 79 | 2833382 | 225 | ------GEDVVFVCNDWHTGPLSCYLKSNYQSHGIYRDAKTAFCIHNISYQGRFAFSDYP | 278 |
| 80 | 297424 | 225 | ------GEDVVFVCNDWHTGPLSCYLKSNYQSHGIYRDAKTAFCIHNISYQGRFAFSDYP | 278 |
| 81 | 7798551 | 225 | ------GEDVVFVCNDWHTGPLSCYLKSNYQSHGIYRDAKTAFCIHNISYQGRFAFSDYP | 278 |
| 82 | 82478 | 225 | ------GEDVVFVCNDWHTGPLSCYLKSNYQSHGIYRDAKTAFCIHNISYQGRFAFSDYP | 278 |
| 83 | 297422 | 222 | ------GEDVVFVCNDWHTGPLSCYLKSNYQSHGIYRDAKTAFCIHNISYQGRFAFSDYP | 275 |
| 84 | 136755 | 218 | ------GEDVVFVCNDWHTGPLSCYLKSNYQSHGIYRDAKTAFCIHNISYQGRFAFSDYP | 271 |
| 85 | 18652407 | 218 | ------GEDVVFVCNDWHTGPLSCYLKSNYQSHGIYRDAKTAFCIHNISYQGRFAFSDYP | 271 |
| 86 | 4760582 | 220 | ------GEDVVFVCNDWHTGPLSCYLKSNYQSHGIYRDAKTAFCIHNISYQGRFAFSDYP | 273 |
| 87 | 11037536 | 220 | ------GEDVVFVCNDWHTGPLSCYLKSNYQSHGIYRDAKTAFCIHNISYQGRFAFSDYP | 273 |
| 88 | 6624287 | 219 | ------GEDVVFVCNDWHTGPLSCYLKSNYQSHGIYRDAKTAFCIHNISYQGRFAFSDYP | 272 |
| 89 | 6624283 | 220 | ------GEDVVFVCNDWHTGPLSCYLKSNYQSHGIYRDAKTAFCIHNISYQGRFAFSDYP | 273 |
| 90 | 4760584 | 219 | ------GEDVVFVCNDWHTGPLSCYLKSNYQSHGIYRDAKTAFCIHNISYQGRFAFSDYP | 272 |
| 91 | 6318538 | 220 | ------GEDVVFVCNDWHTGPLSCYLKSNYQSHGIYRDAKTAFCIHNISYQGRFAFSDYP | 273 |
| 92 | 6318540 | 220 | ------GEDVVFVCNDWHTGPLSCYLKSNYQSHGIYRDAKTAFCIHNISYQGRFAFSDYP | 273 |
| 93 | 17736918 | 149 | ------GEDVVFVCNDWHTGPLSCYLKSNYQSHGIYRDAKTAFCIHNISYQGRFAFSDYP | 202 |
| 94 | 6624285 | 219 | ------GEDVVFVCNDWHTGPLSCYLKSNYQSHGIYRDAKTAFCIHNISYQGRFAFSDYP | 272 |
| 95 | 6624281 | 219 | ------GEDVVFVCNDWHTGPLSCYLKSNYQSHGIYRDAKTAFCIHNISYQGRFAFSDYP | 272 |
| 96 | 4760580 | 219 | ------GEDVVFVCNDWHTGPLSCYLKSNYQSHGIYRDAKTAFCIHNISYQGRFAFSDYP | 272 |
| 97 | 136765 | 224 | AVPRRAGEDVVFVCNDWHTGPLSCYLKSNYQSHGIYRDAKTAFCIHNISYQGRFAFSDYP | 283 |
| 98 | 4588609 | 219 | ------GEDVVFVCNDWHTGPLSCYLKSNYQSHGIYRDAKTAFCIHNISYQGRFAFSDYP | 272 |
| 99 | 4588607 | 180 | ------GEDVVFVCNDWHTGPLSCYLKSNYQSHGIYRDAKTAFCIHNISYQGRFAFSDYP | 233 |
| 100 | 6136121 | 224 | ------GEDVVFVCNDWHTGPLSCYLKSNYQSHGIYRDAKTAFCIHNISYQGRFAFSDYP | 277 |
| 101 | 15637079 | 224 | ------GEDVVFVCNDWHTGPLSCYLKSNYQSHGIYRDAKTAFCIHNISYQGRFAFSDYP | 277 |
| 102 | 3832512 | 223 | ------GEDVVFVCNDWHTGPLSCYLKSNYQSHGIYRDAKTAFCIHNISYQGRFAFSDYP | 276 |
| 103 | 2833381 | 224 | ------GEDVVFVCNDWHTGPLSCYLKSNYQSHGIYRDAKTAFCIHNISYQGRFAFSDYP | 277 |
| 104 | 12003285 | 220 | ------GEDVVFVCNDWHTGPLSCYLKSNYQSHGIYRDAKTAFCIHNISYQGRFAFSDYP | 273 |
| 105 | 228210 | 223 | ------GEDVVFVCNDWHTGPLSCYLKSNYQSHGIYRDAKTAFCIHNISYQGRFAFSDYP | 276 |
| 106 | 15626365 | 229 | ------GEDVVFVCNDWHTGPLSCYLKSNYQSHGIYRDAKTAFCIHNISYQGRFAFSDYP | 282 |
| 107 | 267196 | 223 | ------GEDVVFVCNDWHTGPLSCYLKSNYQSHGIYRDAKTAFCIHNISYQGRFAFSDYP | 276 |
| 108 | 2833388 | 224 | ------GEDVVFVCNDWHTGPLSCYLKSNYQSHGIYRDAKTAFCIHNISYQGRFAFSDYP | 277 |
| 109 | 602594 | 223 | ------GEDVVFVCNDWHTGPLSCYLKSNYQSHGIYRDAKTAFCIHNISYQGRFAFSDYP | 276 |
| 110 | 15223331 | 226 | ------GEDVVFVCNDWHTGPLSCYLKSNYQSHGIYRDAKTAFCIHNISYQGRFAFSDYP | 279 |
| 111 | 5441242 | 222 | ------GEDVVFVCNDWHTGPLSCYLKSNYQSHGIYRDAKTAFCIHNISYQGRFAFSDYP | 275 |
| 112 | 18139611 | 223 | ------GEDVVFVCNDWHTGPLSCYLKSNYQSHGIYRDAKTAFCIHNISYQGRFAFSDYP | 276 |
| 113 | 2833383 | 219 | ------GEDVVFVCNDWHTGPLSCYLKSNYQSHGIYRDAKTAFCIHNISYQGRFAFSDYP | 272 |
| 114 | 6492245 | 214 | ------GEDVVFVCNDWHTGPLSCYLKSNYQSHGIYRDAKTAFCIHNISYQGRFAFSDYP | 267 |
| 115 | 9587307 | 127 | ------GEDVVFVCNDWHTGPLSCYLKSNYQSHGIYRDAKTAFCIHNISYQGRFAFSDYP | 180 |
| 116 | 9587337 | 127 | ------GEDVVFVCNDWHTGPLSCYLKSNYQSHGIYRDAKTAFCIHNISYQGRFAFSDYP | 180 |
| 117 | 9587327 | 127 | ------GEDVVFVCNDWHTGPLSCYLKSNYQSHGIYRDAKTAFCIHNISYQGRFAFSDYP | 180 |
| 118 | 9587333 | 127 | ------GEDVVFVCNDWHTGPLSCYLKSNYQSHGIYRDAKTAFCIHNISYQGRFAFSDYP | 180 |
| 119 | 9587329 | 127 | ------GEDVVFVCNDWHTGPLSCYLKSNYQSHGIYRDAKTAFCIHNISYQGRFAFSDYP | 180 |
| 120 | 9587325 | 127 | ------GEDVVFVCNDWHTGPLSCYLKSNYQSHGIYRDAKTAFCIHNISYQGRFAFSDYP | 180 |

TABLE IX
Maize (GBSS) “LINKR” Domain and it's Alignments with other similar proteins

| SEQ | Accession | a.a | | a.a. |
|---|---|---|---|---|
| Id.No. | Number | # | (start) “LINKR” Sequence (ending) | # |
| 121 | MAIZE GESS | 273 | ELNLPERFKSSFDFIDGYEKPVEGRKINWMKAGILEADRVLTVSPYYAEELISGIARGCE | 332 |
| 122 | 136757 | 273 | ELNLPERFKSSFDFIDGYEKPVEGRKINWMKAGILEADRVLTVSPYYAEELISGIARGCE | 332 |
| 123 | 2833385 | 278 | ELNLPERFKSSFDFIDGYEKPVEGRKINWMKAGILEADRVLTVSPYYAEELISGIARGCE | 337 |
| 124 | 136758 | 279 | ELNLPERFKSSFDFIDGYEKPVEGRKINWMKAGILEADRVLTVSPYYAEELISGIARGCE | 338 |
| 125 | 2833382 | 279 | ELNLPERFKSSFDFIDGYEKPVEGRKINWMKAGILEADRVLTVSPYYAEELISGIARGCE | 338 |
| 126 | 297424 | 279 | ELNLPERFKSSFDFIDGYEKPVEGRKINWMKAGILEADRVLTVSPYYAEELISGIARGCE | 338 |
| 127 | 7798551 | 279 | ELNLPERFKSSFDFIDGYEKPVEGRKINWMKAGILEADRVLTVSPYYAEELISGIARGCE | 338 |
| 128 | 82478 | 279 | ELNLPERFKSSFDFIDGYEKPVEGRKINWMKAGILEADRVLTVSPYYAEELISGIARGCE | 338 |
| 129 | 297422 | 276 | ELNLPERFKSSFDFIDGYEKPVEGRKINWMKAGILEADRVLTVSPYYAEELISGIARGCE | 335 |
| 130 | 136755 | 272 | QLNLPDRFKSSFDFIDGYDKPVEGRKINWMKAGILQADKVLTVSPYYAEELISGEARGCE | 331 |
| 131 | 118652407 | 272 | QLNLPDRFKSSFDFIDGYDKPVEGRKINWMKAGILQADKVLTVSPYYAEELISGEARGCE | 331 |
| 132 | 4760582 | 274 | QLNLPDRFKSSFDFIDGYDKPVEGRKINWMKAGILQADKVLTVSPYYAEELISGEARGCE | 333 |
| 133 | 11037536 | 274 | QLNLPDRFKSSFDFIDGYDKPVEGRKINWMKAGILQADKVLTVSPYYAEELISGEARGCE | 333 |
| 134 | 6624287 | 273 | QLNLPDRFKSSFDFIDGYDKPVEGRKINWMKAGILQADKVLTVSPYYAEELISGEARGCE | 332 |
| 135 | 6624283 | 274 | QLNLPDRFKSSFDFIDGYDKPVEGRKINWMKAGILQADKVLTVSPYYAEELISGEARGCE | 333 |
| 136 | 4760584 | 273 | QLNLPDRFKSSFDFIDGYDKPVEGRKINWMKAGILQADKVLTVSPYYAEELISGEARGCE | 332 |
| 137 | 6318538 | 274 | QLNLPDRFKSSFDFIDGYDKPVEGRKINWMKAGILQADKVLTVSPYYAEELISGEARGCE | 333 |
| 138 | 6318540 | 274 | QLNLPDRFKSSFDFIDGYDKPVEGRKINWMKAGILQADKVLTVSPYYAEELISGEARGCE | 333 |
| 139 | 17736918 | 203 | QLNLPDRFKSSFDFIDGYDKPVEGRKINWMKAGILQADKVLTVSPYYAEELISGEARGCE | 262 |
| 140 | 6624285 | 273 | QLNLPDRFKSSFDFIDGYDKPVEGRKINWMKAGILQADKVLTVSPYYAEELISGEARGCE | 332 |
| 141 | 6624281 | 273 | QLNLPDRFKSSFDFIDGYDKPVEGRKINWMKAGILQADKVLTVSPYYAEELISGEARGCE | 332 |
| 142 | 4760580 | 273 | QLNLPDRFKSSFDFIDGYDKPVEGRKINWMKAGILQADKVLTVSPYYAEELISGEARGCE | 332 |
| 143 | 136765 | 284 | QLNLPDRFKSSFDFIDGYDKPVEGRKINWMKAGILQADKVLTVSPYYAEELISGEARGCE | 343 |
| 144 | 4588609 | 273 | QLNLPDRFKSSFDFIDGYDKPVEGRKINWMKAGILQADKVLTVSPYYAEELISGEARGCE | 332 |
| 145 | 4588607 | 234 | QLNLPDRFKSSFDFIDGYDKPVEGRKINWMKAGILQADKVLTVSPYYAEELISGEARGCE | 293 |
| 146 | 6136121 | 278 | LLNLPDQFKSSFDFFDGYEKPVKGRKINWMKAGILESDRVVTVSPYYAKELVSGPDKGVE | 337 |
| 147 | 15637079 | 278 | LLNLPDQFKSSFDFFDGYEKPVKGRKINWMKAGILESDRVVTVSPYYAKELVSGPDKGVE | 337 |
| 148 | 3832512 | 277 | LLNLPDQFKSSFDFFDGYEKPVKGRKINWMKAGILESDRVVTVSPYYAKELVSGPDKGVE | 336 |
| 149 | 2833381 | 278 | LLNLPDQFKSSFDFFDGYEKPVKGRKINWMKAGILESDRVVTVSPYYAKELVSGPDKGVE | 337 |
| 150 | 12003285 | 274 | KLNLPDQLKSSFDFMDGYEKPVKGRKINWMKAGIIESDRVLTVSPYYANELVSGPDKGVE | 333 |
| 151 | 228210 | 277 | LLNLPDQFKSSFDFFDGYEKPVKGRKINWMKAGILESDRVVTVSPYYAKELVSGPDKGVE | 336 |
| 152 | 15626365 | 283 | LLNLPDQFKSSFDFFDGYEKPVKGRKINWMKAGILESDRVVTVSPYYAKELVSGPDKGVE | 342 |
| 153 | 267196 | 277 | LLNLPDQFKSSFDFFDGYEKPVKGRKINWMKAGILESDRVVTVSPYYAKELVSGPDKGVE | 336 |
| 154 | 2833388 | 278 | RLNLPDQFKSSFDFFDGYEKPVKGRKINWMKAGILESDRVVTVSPYYAKELVSGPDKGVE | 337 |
| 155 | 602594 | 277 | LLNLPDQFKSSFDFFDGYEKPVKGRKINWMKAGILESDRVVTVSPYYAKELVSGPDKGVE | 336 |
| 156 | 15223331 | 280 | LLNLPDQFKSSFDFFDGYEKPVKGRKINWMKAGILESDRVVTVSPYYAKELVSGPDKGVE | 339 |
| 157 | 5441242 | 276 | LLNLPDQFKSSFDFFDGYEKPVKGRKINWMKAGILESDRVVTVSPYYAKELVSGPDKGVE | 335 |
| 158 | 18139611 | 277 | LLNLPDQFKSSFDFFDGYEKPVKGRKINWMKAGILESDRVVTVSPYYAKELVSGPDKGVE | 336 |
| 159 | 2833383 | 273 | LLNLPDQFKSSFDFFDGYEKPVKGRKINWMKAGILESDRVVTVSPYYAKELVSGPDKGVE | 332 |
| 160 | 6492245 | 268 | LLNLPDQFKSSFDFFDGYEKPVKGRKINWMKAGILESDRVVTVSPYYAKELVSGPDKGVE | 327 |
| 161 | 9587307 | 181 | LLNLPDQFKSSFDFFDGYEKPVKGRKINWMKAGILESDRVVTVSPYYAKELVSGPDKGVE | 240 |
| 162 | 9587337 | 181 | LLNLPDQFKSSFDFFDGYEKPVKGRKINWMKAGILESDRVVTVSPYYAKELVSGPDKGVE | 240 |
| 163 | 9587327 | 181 | LLNLPDQFKSSFDFFDGYEKPVKGRKINWMKAGILESDRVVTVSPYYAKELVSGPDKGVE | 240 |
| 164 | 9587333 | 181 | LLNLPDQFKSSFDFFDGYEKPVKGRKINWMKAGILESDRVVTVSPYYAKELVSGPDKGVE | 240 |
| 165 | 9587329 | 181 | LLNLPDQFKSSFDFFDGYEKPVKGRKINWMKAGILESDRVVTVSPYYAKELVSGPDKGVE | 240 |
| 166 | 9587325 | 181 | LLNLPDQFKSSFDFFDGYEKPVKGRKINWMKAGILESDRVVTVSPYYAKELVSGPDKGVE | 240 |
| 121 | MAIZE GESS | 333 | LDNIMRLTGI-TGIVNGMDVSEWDPSRDKYIAVKYDVSTAVEAKALNKEALQAEVGLPVD | 391 |
| 122 | 136757 | 333 | LDNIMRLTGI-TGIVNGMDVSEWDPSRDKYIAVKYDVSTAVEAKALNKEALQAEVGLPVD | 391 |
| 123 | 2833385 | 338 | LDNIMRLTGI-TGIVNGMDVSEWDPSRDKYIAVKYDVSTAVEAKALNKEALQAEVGLPVD | 396 |
| 124 | 136758 | 339 | LDNIMRLTGI-TGIVNGMDVSEWDPSRDKYIAVKYDVSTAVEAKALNKEALQAEVGLPVD | 397 |
| 125 | 2833382 | 339 | LDNIMRLTGI-TGIVNGMDVSEWDPSRDKYIAVKYDVSTAVEAKALNKEALQAEVGLPVD | 397 |
| 126 | 297424 | 339 | LDNIMRLTGI-TGIVNGMDVSEWDPSRDKYIAVKYDVSTAVEAKALNKEALQAEVGLPVD | 397 |
| 127 | 7798551 | 339 | LDNIMRLTGI-TGIVNGMDVSEWDPSRDKYIAVKYDVSTAVEAKALNKEALQAEVGLPVD | 397 |
| 128 | 82478 | 339 | LDNIMRLTGI-TGIVNGMDVSEWDPSRDKYIAVKYDVSTAVEAKALNKEALQAEVGLPVD | 397 |
| 129 | 297422 | 336 | LDNIMRLTGI-TGIVNGMDVSEWDPSRDKYIAVKYDVSTAVEAKALNKEALQAEVGLPVD | 394 |
| 130 | 136755 | 332 | LDNIMRLTGI-TGIVNGMDVSEWDPSRDKYIAVKYDVSTAVEAKALNKEALQAEVGLPVD | 390 |
| 131 | 18652407 | 332 | LDNIMRLTGI-TGIVNGMDVSEWDPSRDKYIAVKYDVSTAVEAKALNKEALQAEVGLPVD | 390 |
| 132 | 4760582 | 334 | LDNIMRLTGI-TGIVNGMDVSEWDPSRDKYIAVKYDVSTAVEAKALNKEALQAEVGLPVD | 392 |
| 133 | 11037536 | 334 | LDNIMRLTGI-TGIVNGMDVSEWDPSRDKYIAVKYDVSTAVEAKALNKEALQAEVGLPVD | 392 |
| 134 | 6624287 | 333 | LDNIMRLTGI-TGIVNGMDVSEWDPSRDKYIAVKYDVSTAVEAKALNKEALQAEVGLPVD | 391 |
| 135 | 662428 | 334 | LDNIMRLTGI-TGIVNGMDVSEWDPSRDKYIAVKYDVSTAVEAKALNKEALQAEVGLPVD | 392 |
| 136 | 4760584 | 333 | LDNIMRLTGI-TGIVNGMDVSEWDPSRDKYIAVKYDVSTAVEAKALNKEALQAEVGLPVD | 391 |
| 137 | 6318538 | 334 | LDNIMRLTGI-TGIVNGMDVSEWDPSRDKYIAVKYDVSTAVEAKALNKEALQAEVGLPVD | 392 |
| 138 | 6318540 | 334 | LDNIMRLTGI-TGIVNGMDVSEWDPSRDKYIAVKYDVSTAVEAKALNKEALQAEVGLPVD | 392 |
| 139 | 17736918 | 263 | LDNIMRLTGI-TGIVNGMDVSEWDPSRDKYIAVKYDVSTAVEAKALNKEALQAEVGLPVD | 321 |
| 140 | 6624285 | 333 | LDNIMRLTGI-TGIVNGMDVSEWDPSRDKYIAVKYDVSTAVEAKALNKEALQAEVGLPVD | 391 |
| 141 | 6624281 | 333 | LDNIMRLTGI-TGIVNGMDVSEWDPSRDKYIAVKYDVSTAVEAKALNKEALQAEVGLPVD | 391 |
| 142 | 4760580 | 333 | LDNIMRLTGI-TGIVNGMDVSEWDPSRDKYIAVKYDVSTAVEAKALNKEALQAEVGLPVD | 391 |
| 143 | 136765 | 344 | LDNIMRLTGI-TGIVNGMDVSEWDPSRDKYIAVKYDVSTAVEAKALNKEALQAEVGLPVD | 402 |
| 144 | 4588609 | 333 | LDNIMRLTGI-TGIVNGMDVSEWDPSRDKYIAVKYDVSTAVEAKALNKEALQAEVGLPVD | 391 |
| 145 | 4588607 | 294 | LDNIMRLTGI-TGIVNGMDVSEWDPSRDKYIAVKYDVSTAVEAKALNKEALQAEVGLPVD | 352 |
| 146 | 6136121 | 338 | LDNVIAKTSI-TGIVNGMDVSEWDPSRDKYIAVKYDVSTAVEAKALNKEALQAEVGLPVD | 396 |
| 147 | 15637079 | 338 | LDNHIRDCGI-TGIVNGMDVSEWDPSRDKYIAVKYDVSTAVEAKALNKEALQAEVGLPVD | 396 |
| 148 | 3832512 | 337 | LDNIIRSIGI-TGIVNGMDVSEWDPSRDKYIAVKYDVSTAVEAKALNKEALQAEVGLPVD | 395 |
| 149 | 2833381 | 338 | LDNHIRDCGI-TGIVNGMDVSEWDPSRDKYIAVKYDVSTAVEAKALNKEALQAEVGLPVD | 396 |
| 150 | 12003285 | 334 | LDNILRKCTV-TGIVNGMDTQEWNPATDKYIDNHYDITTDGKPLLKEALQAEVGLPVD | 392 |
| 151 | 228210 | 337 | LDSVLRKTCI-TGIVNGMDTQEWNPATDKYIDNHYDITTDGKPLLKEALQAEVGLPVD | 395 |
| 152 | 15626365 | 343 | LDNILRRVGV-TGIVNGMDTQEWNPATDKYIDNHYDITTDGKPLLKEALQAEVGLPVD | 401 |
| 153 | 267196 | 337 | LDSVLRKTCI-TGIVNGMDTQEWNPATDKYIDNHYDITTDGKPLLKEALQAEVGLPVD | 395 |
| 154 | 2833388 | 338 | LDNFIRKTGI-AGIINGMDVQEWNPVTDKYIDIHYDATTVMDAKPLLQAEVGLPVD | 396 |
| 155 | 602594 | 337 | LDSVLRKTCI-TGIVNGMDTQEWNPATDKYIDNHYDITTDGKPLLKEALQAEVGLPVD | 395 |
| 156 | 15223331 | 340 | LHKYLRMKTV-SGIINGMDVQEWNPSTDKYIDIKYDITTVTDAKPLIKEALQAAVGLPVD | 398 |
| 157 | 5441242 | 336 | LDNIIRKTGV-AGIVNGMDIREWSPKTDKFIDIHFDTTSVKEAKFLLQAEVGLPVN | 394 |
| 158 | 18139611 | 337 | LDGILRTKPLETGIVNGMDVYEWNPATDQYISVKYDATTVTEARALNKEMLQAEVGLPVD | 396 |
| 159 | 2833383 | 333 | LDNIIRSTGI-IGIVNGMDNREWSPQTDRYIDETTVTEAKPLLKGTLQAEIGLPVD | 391 |
| 160 | 6492245 | 328 | LDGVLRAKPLETGIVNGMDVVDWNPATDKYISVKYNATAKEILQAEVGLPVD | 387 |
| 161 | 9587307 | 241 | LDNIIRKTGI-QFGIVNGMVQEWNPLTDKYTTVKYDASTVADAKPLLKEALQAEVGLPVD | 299 |
| 162 | 9587337 | 241 | LDNIIRKTGI-QFGIVNGMVQEWNPLTDKYTTVKYDASTVADAKPLLKEALQAEVGLPVD | 299 |
| 163 | 9587327 | 241 | LDNIIRKTGI-QFGIVNGMVQEWNPLTDKYTTVKYDASTVADAKPLLKEALQAEVGLPVD | 299 |
| 164 | 9587333 | 241 | LDNIIRKTGI-QFGIVNGMVQEWNPLTDKYTTVKYDASTVADAKPLLKEALQAEVGLPVD | 299 |
| 167 | 150549 | 221 | NIMRLTGI-TGIVNGMDVSEWDPSRDKYIAVKYDVSTAVEAKALNKEALQAEVGLPVD | 57 |
| 165 | 9587329 | 241 | NIMRLTGI-TGIVNGMDVSEWDPSRDKYIAVKYDVSTAVEAKALNKEALQAEVGLPVD | 299 |
| 168 | 15054954 | 1 | NIMRLTGI-TGIVNGMDVSEWDPSRDKYIAVKYDVSTAVEAKALNKEALQAEVGLPVD | 57 |
| 169 | 15054970 | 1 | NIMRLTGI-TGIVNGMDVSEWDPSRDKYIAVKYDVSTAVEAKALNKEALQAEVGLPVD | 57 |
| 170 | 15054956 | 1 | NIMRLTGI-TGIVNGMDVSEWDPSRDKYIAVKYDVSTAVEAKALNKEALQAEVGLPVD | 57 |
| 171 | 15054960 | 1 | NIMRLTGI-TGIVNGMDVSEWDPSRDKYIAVKYDVSTAVEAKALNKEALQAEVGLPVD | 57 |
| 166 | 958732524 | 1 | LDNIIRKTGI-RGIVNGMDVQEWNPLTDKYTIVKYDASTVTDAKPLLKEASQAEVGLFVD | 299 |

TABLE X
Maize (GBSS) “GLYTR” Domain and it's Alignments with other proteins

| SEQ | Accession | a.a | | a.a. |
|---|---|---|---|---|
| Id.No. | Number | # | (start) “GLYTR” Sequence (ending) | # |
| 172 | MAIZE GBSS | 392 | RNIPLVAFIGRLEEQKGPDVMAAAIPQLMEMVEDVQIVLLGTGKKKFERMLMSAEEKFPG | 451 |
| 173 | 136757 | 392 | RNIPLVAFIGRLEEQKGPDVMAAAIPQLMEMVEDVQIVLLGTGKKKFERMLMSAEEKFPG | 451 |
| 174 | 2833385 | 397 | RKIPLVAFIGRLEEQKGPDVMAAAIPQLME--EDVQIVLLGTGKKKFERMLMSAEEKFPG | 454 |
| 175 | 136758 | 398 | RKIPLVAFIGRLEEQKGPDVMAAAIPQLMQ--EDVQIVLLGTGKKKFERMLMSAEEKFPG | 455 |
| 176 | 2833382 | 398 | RKIPLVAFIGRLEEQKGPDVMAAAIPQLMQ--EDVQIVLLGTGKKKFERMLMSAEEKFPG | 455 |
| 177 | 297424 | 398 | RKIPLVAFIGRLEEQKGPDVMAAAIPQLMQ--EDVQIVLLGTGKKKFERMLMSAEEKFPG | 455 |
| 178 | 7798551 | 398 | RKIPLVAFIGRLEEQKGPDVMAAAIPQLMQ--EDVQIVLLGTGKKKFERMLMSAEEKFPG | 455 |
| 179 | 82478 | 398 | RKIPLIAFIGRLEEQKGPDVMAAAIPELMQ--EDVQIVLLGTGKKKFEKLLKSMEEKYPG | 455 |
| 180 | 297422 | 395 | RKVPLIAFIGRLEEQKGPDVMAAAIPELMQ--ENVQIVLLGTGKKKFEKLLKSMEEKYPG | 452 |
| 181 | 136755 | 391 | RKVFLVAFIGRLEEQKGPDVMIAAIPEILKE-EDVQIILLGTGKKKFEKLLKSMEEKFPG | 449 |
| 182 | 18652407 | 391 | RKVFLVAFIGRLEEQKGPDVMIAAIPEILKE-EDVQIILLGTGKKKFEKLLKSMEEKFPG | 449 |
| 183 | 4760582 | 393 | RKVFLVAFIGRLEEQKGPDVMIAAIPEILKE-EDVQIILLGTGKKKFEKLLKSMEEKFPD | 451 |
| 184 | 11037536 | 393 | RKVFLVAFIGRLEEQKGPDVMIAAIPEILKE-EDVQIILLGTGKKKFEKLLKSMEEKFPS | 451 |
| 185 | 6624287 | 392 | RKVFLVAFIGRLEEQKGPDVMIAAIPEILKE-EDVQIILLGTGKKKFEKLLKSMEEKFPN | 450 |
| 186 | 6624283 | 393 | RKVFLVAFIGRLEEQKGPDVMIAAIPEILKE-EDVQIILLGTGKKKFEKLLKSMEEKFPS | 451 |
| 187 | 4760584 | 392 | RKVFLVAFIGRLEEQKGPDVMIAAIPEILKE-EDVQIILLGTGKKKFEKLLKSMEEKFPS | 450 |
| 188 | 6318538 | 393 | RKVFLVAFIGRLEEQKGPDVMIAAIPEILKE-EDVQIILLGTGKKKFEKLLKSMEEKFPS | 451 |
| 189 | 6318540 | 393 | RKVFLVAFIGRLEEQKGPDVMIAAIPEILKE-EDVQIILLGTGKKKFEKLLKSMEEKFPS | 451 |
| 190 | 17736918 | 322 | RKVFLVAFIGRLEEQKGPDVMIAAIPEILKE-EDVQIILLGTGKKKFEKLLKSMEEKFPS | 380 |
| 191 | 6624285 | 392 | RKVFLVAFIGRLEEQKGPDVMIAAIPEILKE-EDVQIILLGTGKKKFEKLLKSMEEKFPT | 450 |
| 192 | 6624281 | 392 | RKVFLVAFIGRLEEQKGPDVMIAAIPEILKE-EDVQIILLGTGKKKFEKLLKSMEEKFPT | 450 |
| 193 | 4760580 | 392 | RKVFLVAFIGRLEEQKGPDVMIAAIPEILKE-EDVQIILLGTGKKKFEKLLKSMEEKFPT | 450 |
| 194 | 136765 | 403 | RKVFLVAFIGRLEEQKGPDVMIAAIPEILKE-EDVQIILLGTGKKKFEKLLKSMEEKFPT | 461 |
| 195 | 4588609 | 392 | RKVFLVAFIGRLEEQKGPDVMIAAIPEILKE-EDVQIILLGTGKKKFEKLLKSMEEKFPS | 450 |
| 196 | 4588607 | 353 | RKVFLVAFIGRLEEQKGPDVMIAAIPEILKE-EDVQIILLGTGKKKFEKLLKSMEEKFPT | 411 |
| 197 | 6136121 | 397 | KNIPVIGFIGRLEEQKGSDILVAAISKFVGL--DVQIIILGTGKKKFEQQIQELEVLYPD | 454 |
| 198 | 15637079 | 397 | RNIPLIGFIGRLEEQKGSDILYAAISKFISM--DVQILILGTGKKKFEQQIEQLEVMYPD | 454 |
| 199 | 3832512 | 396 | RNIPVIGFIGRLEEQKGSDILVESIPKFID--QNVQIIVLGTGKKIMEKQIEQLEVTYPG | 453 |
| 200 | 2833381 | 397 | RNIPLIGFIGRLEEQKGSDILYAAISKFISM--DVQILILGTGKKKFEQQIEQLEVMYPD | 454 |
| 201 | 12003285 | 393 | PNVPLVGFIGRLEEQKGSDILVAALHKFIEM--DVQVVILGTGKKEFEKQIEQLEELYPG | 450 |
| 202 | 228210 | 396 | KKIPLIGFIGRLEEQKGSDILVAAIHKFIGL--DVQIVVLGTGKKEFEQEIEQLEVLYPG | 453 |
| 203 | 15626365 | 402 | KNVPLIAFIGRLEEQKGSDILVEAIPQFIK--ENVQIVALGTGKKEMEKQLQQLEISYPD | 459 |
| 204 | 267196 | 396 | KKIPLIGFIGRLEEQKGSDILVAAIHKFIGL--DVQIVVLGTGKKEFEQEIEQLEVLYPN | 453 |
| 205 | 2833388 | 397 | PNVPLIGFIGRLEEQKGSDIFVAAISQLVE--HNVQIVILGTGKKKFEKQIEHLEVLYPD | 454 |
| 206 | 602594 | 396 | KKVPLIGFIGRLEEQKGSDILVAAIHKFIGL--DVQIVVLGTGKKEFEQEIEQLEVLYPN | 453 |
| 207 | 15223331 | 399 | RDVPVIGFIGRLEEQKGSDILVEAISKFMGL--NVQMVILGTGKKKMEAQILELEEKFFG | 456 |
| 208 | 5441242 | 395 | RDIPLIGFIGRLEEQKGSDILVEAIPKFID--QNVQIIILGTGKKSMEKQIEQLEEIYPE | 452 |
| 209 | 18139611 | 397 | SSIPLIVFVGRLEEQKGSDILIAAIPEFVE--GNVQIIVLGTGKKKMEEELILLEVKYPN | 454 |
| 210 | 2833383 | 392 | SSIPLIGFIGRLEEQKGSDILVEAIAKFAD--ENVQIVVLGTGKKIMEKQIEVLEEKYPG | 449 |
| 211 | 6492245 | 388 | SSIFVIVFIGRLEEQKGSDILIAAIPEFLE--ENVQIIVLGTGKKKMEEELMLLEAKYPQ | 445 |
| 212 | 9587307 | 300 | RDIPVIGFIGRLEE | 313 |
| 213 | 9587337 | 300 | RDIFVIGFIGRLEE | 313 |
| 214 | 9587327 | 300 | KDIPVIGFIGRLEE | 313 |
| 215 | 9587333 | 300 | KMIPVIGFIGRLEE | 313 |
| 216 | 15054922 | 58 | RNIPLVAFIGRLEEQKGPDVMAAAIPQLMEMVEDVQIVLLGTGKKKFEPMLMSAEEKFFG | 117 |
| 217 | 9587329 | 300 | RDIPVIGFIGRLEE | 313 |
| 218 | 15054954 | 58 | RNIPLVAFIGRLEEQKGPDVMAAAIPQLMEMVEDVQIVLLGTGKKKFERMLMSAEEKFPG | 117 |
| 219 | 15054970 | 58 | RNIPLVAFIGRLEEQKGPDVMAAAIPQLMDMVEDVQIVLLGTGKKKFERMLMSAEEKFPG | 117 |
| 220 | 15054956 | 58 | PNIPLVAEIGRLEEQKGPDVMAAAIPQLMEMVEDVQIVLLGTGKKKFERMLMSAEEKFPG | 117 |
| 221 | 15054960 | 58 | RNIPLVAFIGRLEEQKGPDVMAAAIPQLMEMVEDVQIVLLGTGKKKFERMLMSAEEKFPG | 117 |
| 222 | 9587325 | 300 | RDIPVIGFIGRLEE | 313 |
| 172 | MAIZE GBSS | 452 | KVRAVVKFNAALAHHIMAGADVLAVTSRFEPCGLIQLQGMRYGTPCACASTGGLVDTIIE | 511 |
| 173 | 136757 | 452 | KVRAVVKFNAALAHHIMAGADVLAVTSRFEPCGLIQLQGMRYGTPCACASTGGLVDTIIE | 511 |
| 174 | 2833385 | 455 | KVRAVVKFNAPLAHHIMAGADVLAVTSRFEPCGLIQLQGMRYGTPCACASTGGLVDTIIE | 514 |
| 175 | 136758 | 456 | KVRAVVKFNAPLAHLIMAGADVLAVPSRFEPCGLIQLQGMRYGTPCACASTGGLVDTVIE | 515 |
| 176 | 2833382 | 456 | KVRAVVKFNAPLAHLIMAGADVLAVPSRFEPCGLIQLQGMRYGTPCACASTGGLVDTVIE | 515 |
| 177 | 297424 | 456 | KVRAVVKFNAPLAHLIMAGADVLAVPSRFEPCGLIQLQGMRYGTPCACASTGGLVDTVIE | 515 |
| 178 | 7798551 | 456 | KVRAVVKFNAPLAHLIMAGADVLAVPSRFEPCGLIQLQGMRYGTPCACASTGGLVDTVIE | 515 |
| 179 | 82478 | 456 | KVRAVVKFNAPLAHLIMAGADVLAVPSRFEPCGLIQLQGMRYGTPCACASTGGLVDTVIE | 515 |
| 180 | 297422 | 453 | KVRAVVKFNAPLAHLIMAGADVLAVPSRFEPCGLIQLQGMRYGTPCACASTGGLVDTVIE | 512 |
| 181 | 136755 | 450 | KVRAVVRFNAPLAHQMMAGADLLAVTSRFEPCGLIQLQGMRYGTPCVCASTGGLVDTIVE | 509 |
| 182 | 18652407 | 450 | KVRAVVRFNAPLAHQMMAGADLLAVTSRFEPCGLIQLQGMRYGTPCVCASTGGLVDTIVE | 509 |
| 183 | 4760582 | 452 | KVRAVVRFNAPLAHQMMAGADVLAVTSRFEPCGLIQLQGMRYGTPCACASTGGLVDTIME | 511 |
| 184 | 11037536 | 452 | KVRAVVRFNAPLAHQMMAGADVLAVTSRFEPCGLIQLQGMRYGTPCACASTGGLVDTIME | 511 |
| 185 | 6624287 | 451 | KVRAVVRFNAPLAHQMMAGADVLAVTSRFEPCGLIQLQGMRYGTPCACASTGGLVDTIVE | 510 |
| 186 | 6624283 | 452 | KVRAVVRFNAPLAHQMMAGADVLAVTSRFEPCGLIQLQGMRYGTPCACASTGGLVDTIVE | 511 |
| 187 | 4760584 | 451 | KVPAVVRFNAPLAHQMMAGADVLAVTSRFEPCGLIQLQGMRYGTPCACASTGGLVDTIVE | 510 |
| 188 | 6318538 | 452 | KVRAVVRFNAPLAHQMMAGADVLAVTSRFEPCGLIQLQGMRYGTPCACASTGGLVDTIVE | 511 |
| 189 | 6318540 | 452 | KVRAVVRFNAPLAHQMMAGADVLAVTSRFEPCGLIQLQGMRYGTPCACASTGGLVDTIVE | 511 |
| 190 | 17736918 | 381 | KVRAVVRFNAPLAHQMMAGADVLAVTSRFEPCGLIQLQGMRYGTPCACASTGGLVDTIVE | 440 |
| 191 | 6624285 | 451 | KVRAVVRFNAPLAHQMMAGADVLAVTSRFEPCGLIQLQGMRYGTPCACASTGGLVDTIVE | 510 |
| 192 | 6624281 | 451 | KVRAVVRFNAPLAHQNMAGADVIAVTSRFEPCGLIQLQGMRYGTPCACASTGGLVDTIVE | 510 |
| 193 | 4760580 | 451 | KVWAVVRFNAPLAHQNMAGADVLAVTSRFEPCGLIQLQGMRYGTPCACASTGGLVDTIVE | 510 |
| 194 | 136765 | 462 | KVRAVVRFNAPLAHQMMAGADVLAVTSRFEPCGLIQLQGMRYGTPCACASTGGLVDTIVE | 521 |
| 195 | 4588609 | 451 | KVRAVVRFNAPLAHQMMAGADVLAVTSRFEPCGLIQLQGMRYGTPCACASTGGLVDTIVE | 510 |
| 196 | 4588607 | 412 | KVRAVVRFNAPLAHQMMAGADVLAVTSRFEPCGLIQLQGMRYGTPCACASTGGLVDTIVE | 471 |
| 197 | 6136121 | 455 | KARGVAKFNVPLAHMITAGADFMLVPSRFEPCGLIQLHANRYGTIPICASTGGLVDTVTE | 514 |
| 198 | 15637079 | 455 | KARGVAKFNVPLAHMITAGADFMLIPSRFEPCGLIQLHANRYGTPCICASTGGLVDTVKE | 514 |
| 199 | 3832512 | 454 | KAIGVAKFNSPLAHKIIAGADFIVIPSRFEPCGLVQLHAMPYGTVPIVSSTGGLVDTVKE | 513 |
| 200 | 2833381 | 455 | KARGVAKFNVPLAHMITAGADFMLIPSRFEPCGLIQLHAMPYGTPCICASTGGLVDTVKE | 514 |
| 201 | 12003285 | 451 | KAVGVAKFNVPLAHKITAGADFMLVPSRFEPCGLIQLHAMPYGTIPICASTGGLVDTVKE | 510 |
| 202 | 228210 | 454 | KVKGVAKFNVPLAHMITAGADFMLVPSRFEPCGLIQLHAMRYGTVPICASTGGLVDTVKE | 513 |
| 203 | 15626365 | 460 | KARGVAKFNVPLAHMMIAGADFILIPSRFEPCGLIQLQAMRYGTVPIVASTGGLVDTVKE | 519 |
| 204 | 267196 | 454 | KAKGVAKFNVPLAHMITAGADFMLVPSRFEPCGLIQLHAMRYGTVPICASTGGLVDTVKE | 513 |
| 205 | 2833388 | 455 | KARGVAKFNVPLAHMITAGADFMLVPSRFEPCGLIQLHAMRYGTVPIVASTGGLVDTVKE | 514 |
| 206 | 602594 | 454 | KAKGVAKFNVPLAHMITAGADFMLVPSRFEPCGLIQLHAMRYGTVPICASTGGLVDTVKE | 513 |
| 207 | 15223331 | 457 | KAVGVAKFNVPLAHMITAGADFIIVPSRFEPCGLIQLHAMRYGTVPIVASTGGLVDTVKD | 516 |
| 208 | 5441242 | 453 | KARGIAKFDGPLAHKIIAGSDFIMIPSRFEPCGLVQLHSMPYGTVPIVSSTGGLVDTVQE | 512 |
| 209 | 18139611 | 455 | TARGLAKFNVPLAHMMFAGADFIIVPSRFEPCGLIQLQGMRYGVVPICSSTGGLVDTVKE | 514 |
| 210 | 2833383 | 450 | KAIGITKFNSPLAHKIIAGADFIVIPSRFEPCGLVQLHAMPYGTVPIVSSTGGLVDTVKE | 509 |
| 211 | 6492245 | 446 | NARGIAKFNVPLAHMMFAGANFIIVPSRFEPCGLIQLQGMRYGVIPICSSTGGLVDTVSE | 505 |
| 216 | 15054922 | 118 | KVRAVVKFNAALAHHIMAGADVLAVTSRFEPCGLIQLQGMRYGTPCACASTGGLVDTIIE | 177 |
| 218 | 15054954 | 118 | KVRAVVKFNAALAHHIMAGADVLAVTSRFEPCGLIQLQGMRYGTPCACSSTGGLVDTIIE | 177 |
| 219 | 15054970 | 118 | KVRAVVKFNAALAHHINAGADVIAVTSRFEPCGLIQLQGMRYGTPCACASTGGLVDTIIE | 177 |
| 220 | 15054956 | 118 | KVRAVVKFNAALAHNIMAGADVIAVTSRFEPCGLIQLQGMRYGTPCACASTGGLVDTIIE | 177 |
| 221 | 15054960 | 118 | KVRAVVKFNAALAHHIMAGADVLAVTSRFEPCGLIQLQGMRYGTPCACASTGGLVDTIIE | 177 |
| 172 | MAIZE GBSS | 512 | GKTGFHMGRLSVDCNVVEPADVKKVATTLQRAIKVVGTPAYEEMVRNCMIQDLSWKGPAK | 571 |
| 173 | 136757 | 512 | GKTGFHMGRLSVDCNVVEPADVKKVATTLQRAIKVVGTPAYEEMVRNCMIQDLSWKGPAK | 571 |
| 174 | 2833385 | 515 | GKTGFHMGRLSVDCNVVEPADVKKVATTLKRAIKVVGTPAYEEMVKNCMIQDLSWKGPAK | 574 |
| 175 | 136758 | 516 | GKTGFHMGRLSVDCKVVEPSDVKKVAATLKRAIKVGTPAYEEMVVRNCMIQDLSWKGPAK | 575 |
| 176 | 2833382 | 516 | GKTGFHMGRLSVNCKVVEPSDVKKVAATLKRAIKVVGTPAYEEMVRNCMNQDLSWKGPAK | 575 |
| 177 | 297424 | 516 | GKTGFHMGRLSVDCKVVEPSDVKKVAATLKRAIKVVGTPAYEEMVRNCMNQDLSWKGPAK | 575 |
| 178 | 7798551 | 516 | GKTGFHMGRLSVDCKVVEPSDVKKVAATLKRAIKVVGTPAYEEMVRNCMNQDLSWKGPAK | 575 |
| 179 | 82478 | 516 | GKTGFHMGRLSVDGKVVEPSDVKKVAATLKRAIKVVGTPAYEEMVRNCMNQDLSWKGPAK | 575 |
| 180 | 297422 | 513 | GKTGFHMGRLSVDGKVVEPSDVQKVATTLKRAIKIVGTPAYNEMVRNCNNQDLSWKGPAK | 572 |
| 181 | 136755 | 510 | GKTGFHMGRLSVDCNVVEPADVKKVATTLKRAVKVVGTPAYQEMVKNCMIQDLSWKGPAK | 569 |
| 182 | 18652407 | 510 | GKTGFHMGRLSVDCNVVEPADVKKVATTLKRAVKVVGTPAYQEMVKNCMIQDLSWKGPAK | 569 |
| 183 | 4760582 | 512 | GKTGFHMGRLSVDCNVVEPADVKKVVTTLKRAVKVVGTPAYHEMVKNCMIQDLSWKGPAK | 571 |
| 184 | 11037536 | 512 | GKTGFHMGHLSVDCNVVEPADVKKVVTTLKRAVKVVGTPAYHEMVKNCMIQDLSWKGPAK | 571 |
| 185 | 6624287 | 511 | GKTGFHMGRLSVDCNVVEPADVKKVVTTLKRAVKVVGTPAYHGMVKNCMIQDLSWKGPAK | 570 |
| 186 | 6624283 | 512 | GKTGFHMGRLSVDCNVVEPADVKKVVTTLKRAVKVVGTPAYHEMVKNCMIQDLSWKGPAK | 571 |
| 187 | 4760584 | 511 | GKTGFHMGRLSVDCNVVEPADVKKVVTTLKRAVKVVGTPAYHEMVKNCMIQDLSWKGPAK | 570 |
| 188 | 6318538 | 512 | GKTGFHMGRLSVDCNVVEPADVKKVVTTLKRAVKVVGTPAYHEMVKNCMIQDLSWKGPAK | 571 |
| 189 | 6318540 | 512 | GKTGFHMGRLSVDCNVVEPADVKKVVTTLKRAVKVVGTPAYHEMVKNCMIQDLSWKGPAK | 571 |
| 190 | 17736918 | 441 | GKTGFHMGRLSVDCNVVEPADVKKVVTTLKRAVKVVGTPAYHEMVKNCMIQDLSWKGPAK | 500 |
| 191 | 6624285 | 511 | GKTGFHMGRLSVDCNVVEPADVKKVVTTLKRAVKVVGTPAYHEMVKNCMIQDLSWKGPAK | 570 |
| 192 | 6624281 | 511 | GKTGFHMGRLSVDCNVVEPADVKKVVTTLKRAVKVVGTPAYHEMVKNCMIQDLSWKGPAK | 570 |
| 193 | 4760580 | 511 | GKTGFHMGRLSVDCNVVEPADVKKVVTTLKRAVKVVGTPAYHEMVKNCMIQDLSWKGPAK | 570 |
| 194 | 136765 | 522 | GKTGFHMGRLSVDCNVVEPADVKKVVTTLKRAVKVVGTPAYHEMVKNCMIQDLSWKGPAK | 581 |
| 195 | 4588609 | 511 | GKTGFHMGRLSVDCNVVEPADVKKVVTTLKFAVKVVGTPAYHEMVKNCMIQDLSWKGPA | 569 |
| 196 | 4588607 | 472 | GKTGFHMGRLSVDCNVVEPADVKKVVTTLKRAVKVVGTPAYHEMVKNCMIQDLSWKGPAK | 531 |
| 197 | 6136121 | 515 | GFTGFHMGAFNVECATVDPADVQKIATTVERALAAYGSVAYKEMIQNCMAQDLSWKGPAK | 574 |
| 198 | 15637079 | 515 | GYTGFHMGAFNVDCETVDPEDVLKVITTVGRALAMYGTLAFTEMIKNCMSQELSWKGPAK | 574 |
| 199 | 3832512 | 514 | GYTGFHVGAFSVECEAVDPADVEKLATTVNRALKTYGTQALKEMILNCMAQDFSWKGPAK | 573 |
| 200 | 2833381 | 515 | GYTGFHMGAFNVDCETVDPEDVLKVITTVGRALAIYGTLAFTEMIKNCMSQELSWKGPAK | 574 |
| 201 | 12003285 | 511 | GFTGFHMGAFNVECDAVDPADVLKIVKTVGRALEVYGTPAFREMINNCMSLDLSWKGPAK | 570 |
| 202 | 228210 | 514 | GYTGFHMGAFNVECDVVDPADVLKIVTTVARALAVYGTLAFAEMIKNCMSEELSWKEPAK | 573 |
| 203 | 15626365 | 520 | GFTGFHMGSFNVKCDAVDPVDVDAIPKTVTKALGVYGTSAFAEMIKNCMAQELSWKGPAK | 579 |
| 204 | 267196 | 514 | GYTGFHMGAFNVECDVVDPADVLKIVTTVARALAVYGTLAFAEMIKNCMSEELSWKEPAK | 573 |
| 205 | 2833388 | 515 | GYTGFQMGALVECDKIDSAflVAAIVKTVARALGTYATAALREMILNCMAQDLSWKGPAR | 574 |
| 206 | 602594 | 514 | GYTGFHNGAFNVECDVVDPADVLKIVTTVARALAVYGTLAFAEMIKNCMSEELSWKEPAK | 573 |
| 207 | 15223331 | 517 | GYTGFHIGRFNVKCEVVDPDDVIATAKAVTRAVAVYGTSAMQEMVKNCMDQDFSWKGPAR | 576 |
| 208 | 5441242 | 513 | GFTGFHMGAFNVDCEAIDPADVEKIATTVRRALGTYGTVAMEKIIQNCMAQDFSWKGPAK | 572 |
| 209 | 18139611 | 515 | GVTGFHMGLFNVECETVDPVDVTAVASTVKRALKQYNTPAFQEMVQNCMAQDLSWKGPAK | 574 |
| 210 | 2833383 | 510 | GYTGFHAGPFDVECEDVDPDDVDKLAATVKRALKTYGTQAMKQIILNCMAQNFSWKKPAK | 569 |
| 211 | 6492245 | 506 | GVTGFHMGSFNVEFETVDPADVAAVASNVTRALKQYKTPSFHANVQNCMAQDLSWKGPAK | 565 |
| 212 | 15054922 | 178 | GKTGFHMGRLSVDCNVVEPADVKKVATTLQRAIKVVGTPAYEEMVFNCMIQDLSWKGPAK | 237 |
| 218 | 15054954 | 178 | GKTGFHMGRLSVDCNVVEPADVKKVATTLQRAIKVVGTPAYEEMVPNCMIQDLSWKGPAK | 237 |
| 219 | 15054970 | 178 | GKTGFHMGRLSVDCNVVEPADVKKVATTLQRAIKVVGTPAYEEMVRNCMIQDLSWKGPAK | 237 |
| 220 | 15054956 | 178 | GKTGFHMGRFSVDCNVVEPADVKKVATTLQRAIKVVGTPAYEEMVRNCMIQDLSWKGPAK | 237 |
| 221 | 15054960 | 178 | GKTGFHMGRLSVDCNVVEPADVKKVATTLQRAIKVVGTPVYEEMVRNCMIQDLSWKGPAK | 237 |

TABLE XI
Maize (GBSS) “CTEND” Domain and it's Alignments with other proteins

| SEQ Id.No. | Accession Number | a.a # | (start) “CTEND” Sequence (ending) | a.a. # |
|---|---|---|---|---|
| 223 | Maize GESS | 572 | NWENVLLSLXXXXXXXXXXXXXXXXLAKENVAAP | 605 |
| 224 | 136757 | 572 | NWENVLLSLGVAGGEPGVEGEEIAPLAKENVAAP | 605 |
| 225 | 2833385 | 575 | NWENVLLSLGVAGGEPGIEGEEIAPLAKENVAAP | 608 |
| 226 | 136758 | 576 | NWENVLLGLGVAGSAPGIEGDEIAPLAKENVAAP | 609 |
| 227 | 2833382 | 576 | NWENVLLGLGVAGSAPGIEGDEIAPLAKENVAAP | 609 |
| 228 | 297424 | 576 | NWENVLLGLGVAGSAPGIEGDEIAPLAKENVAAP | 609 |
| 229 | 7798551 | 576 | NWENVLLGLGVAGSAPGIEGDEIAPLAKENVAAP | 609 |
| 230 | 82478 | 576 | NWENVLLGLGVAGSAPGIEGDEIAPLAKENVAAP | 609 |
| 231 | 297422 | 573 | NWENVLLGLGVAGSEPGVEGEEIAPLAKENVAAP | 606 |
| 232 | 136755 | 570 | NWEDVLLELGVEGSEPGIVGEEIAPLAKENVAAP | 603 |
| 233 | 18652407 | 570 | NWEDVLLELGVEGSEPGIVGEEIAPLAMENVAAP | 603 |
| 234 | 4760582 | 572 | NWEDVLLELGVEGSEPGVIGEEIAPLAMENVAAP | 605 |
| 235 | 11037536 | 572 | NWEDVLLELGVEGSEPGVIGEEIAPLAMENVAAP | 605 |
| 236 | 6624287 | 571 | NWEDVLLELGVEGSEPGVIGEEIAPLAMENVAAP | 604 |
| 237 | 6624283 | 572 | NWEDVLLELGVEGSEPGVIGEEIAPLAMENVAAP | 605 |
| 238 | 4760584 | 571 | NWEDVLLELGVEGSEPGVIGEEIAPLAMENVAAP | 604 |
| 239 | 6318538 | 572 | NWEHThLELGVEGSEPGIVGEEIAPLAMENVAAP | 605 |
| 240 | 6318540 | 572 | NWEDVLLELGVEGSEPGVIGEEIAPLAMENVAAP | 605 |
| 241 | 17736918 | 501 | NWEDVLLELGVEGSEPGVIGEEIAPLANENVAAP | 534 |
| 242 | 6624285 | 571 | NWEDVLLELGVEGSEPGIVGEEIAPLALENVAAP | 604 |
| 243 | 6624281 | 571 | NWEDVLLELGVEGSEPGIVGEEIAPLALENVAAP | 604 |
| 244 | 4760580 | 571 | NWEDVLLELGVEGSEPGIVGEEIAPLALENVAAP | 604 |
| 245 | 136765 | 582 | NWEDVLLELGVEGSEPGIVGEEIAPLALENVAAP | 615 |
| 246 | 4588607 | 532 | NWEDVLLELGVEGSEPGIVGEEIAPLALENVAAP | 565 |
| 247 | 6136121 | 575 | NWEKMLLSLGVSGSEPGVDGEEIAPLAKENVATP | 608 |
| 248 | 15637079 | 575 | NWETVLLSLGVAGSEPGVEGDEIAPLAKENVATP | 608 |
| 249 | 3832512 | 574 | QWEQALLSLEVAGSEPGIDGEEVAPLAKENVATP | 607 |
| 250 | 2833381 | 575 | NWETVLLSLGVAGSEPGVEGEEIAPLAKENVATP | 608 |
| 251 | 12003285 | 571 | NWETVLLSLGVAGSEPGVEGDEIAPLAKENVATP | 604 |
| 252 | 228210 | 574 | KWETLLLGLGASGSEPGVEGEEIAPLAKENVATP | 607 |
| 253 | 15626365 | 580 | KWEEVLLNLGVPDSEPGIDGQEIAPQAKENVATP | 613 |
| 254 | 267196 | 574 | KWETLLLGLGASGSEPGVEGEEIAPLAKENVATP | 607 |
| 255 | 2833388 | 575 | MWEKMLLDLEVTGSEPGTEGEEIAPLAKENVPTP | 608 |
| 256 | 602594 | 574 | KWETLLLGLGASGSEPGVEGEEIAPLAKENVATP | 607 |
| 257 | 1522333 | 577 | LWEKVLLSLNVAGSEAGTEGEEIAPLAKENVATP | 610 |
| 258 | 5441242 | 573 | QWEKVLFSLDVGRSEAGIEGDEIAPLAKENVATP | 606 |
| 259 | 18139611 | 575 | KWEEVLLGLGVEGSQPGIEGEEVAPLAKENVATP | 608 |
| 260 | 2833383 | 570 | LWEKALLNLEVTGNVAGIDGDEIAPLAKENVATP | 603 |
| 261 | 6492245 | 566 | KWEEALLGLGVEGSQPGIEGEEIAPLAKQNVATP | 599 |
| 262 | 15054922 | 238 | NWENVLLSLGVAGGEPGVEGEEIAPLAKENVAAP | 271 |
| 263 | 15054954 | 238 | NWENVLLSLGVAGGEPGVEGEEIAPLAKENVAAP | 271 |
| 264 | 15054970 | 238 | NWENVLLSLGVAGGEPGVEGEEIAPLAKENVAAP | 271 |
| 265 | 15054956 | 238 | NWENVLLSLGVAGGEPGVEGEEIAPLAKENVAAP | 271 |
| 266 | 15054960 | 238 | NWENVLLSLGVAGGEPGVEGEEIAPLAKENVAAP | 271 |

TABLE XII
Identities of the Accession Numbers used in Table Numbers VIII-XI.

| Accession | Brief Description of sequences | | |
|---|---|---|---|
| Id. | producing significant alignments | score | E-value |
| gi\|136757\|sp\|P04713\|UGST MAIZE | Granule-bound glycogen [star . . . | 1125 | 0.0 |
| gi\|2833385\|sp\|Q43134\|UGST SORBI | Granule-bound glycogen [sta . . . | 1062 | 0.0 |
| gi\|136758\|sp\|P19395\|UGST ORYSA | Granule-bound glycogen [star . . . | 959 | 0.0 |
| gi\|2833382\|sp\|Q42968\|UGST ORYGL | Granule-bound glycogen [sta . . . | 957 | 0.0 |
| gi\|297424\|emb\|CAA46294.1\| | (X65183) glycogen (starch) syntha . . . | 956 | 0.0 |
| gi\|7798551\|gb\|AAC61675.2\| | (AF031162) granule-bound starch s . . . | 956 | 0.0 |
| gi\|82478\|pir\|JQ0703 | UDPglucose--starch glucosyltransferase . . . | 951 | 0.0 |
| gi\|297422\|emb\|CAA45472.1\| | (X64108) starch granule-bound sta . . . | 945 | 0.0 |
| gi\|136755\|sp\|P09842\|UGST HORVU | Granule-bound glycogen [star . . . | 944 | 0.0 |
| gi\|18652407\|gb\|AAL77109.1\|AF474373_6 | (AF474373) granule-bou . . . | 943 | 0.0 |
| gi\|4760582\|dbj\|BAA77351.1\| | (AB019623) starch synthase (GBSS . . . | 939 | 0.0 |
| gi\|11037536\|gb\|AAG27624.1\|AF286320_1 | (AF286320) granule bou . . . | 937 | 0.0 |
| gi\|6624287\|dbj\|BAA88512.1\| | (AB029064) starch synthase (GBSS . . . | 936 | 0.0 |
| gi\|6624283\|dbj\|BAA88510.1\| | (AB029062) starch synthase (GBSS . . . | 935 | 0.0 |
| gi\|4760584\|dbj\|BAA77352.1\| | (AB019624) starch synthase (GBSS . . . | 935 | 0.0 |
| gi\|6318538\|gb\|AAF06936.1\|AF110373_1 | (AF110373) granule-boun . . . | 932 | 0.0 |
| gi\|6318540\|gb\|AAF06937.1\|AF110374_1 | (AF110374) granule-boun . . . | 930 | 0.0 |
| gi\|17736918\|gb\|AAL41028.1\| | (AF250137) mutant granule bound . . . | 929 | 0.0 |
| gi\|6624285\|dbj\|BAA88511.1\| | (AB029063) starch synthase (GBSS . . . | 926 | 0.0 |
| gi\|6624281\|dbj\|BAA88509.1\| | (AB029061) starch synthase (GBSS . . . | 922 | 0.0 |
| gi\|4760580\|dbj\|BAA77350.1\| | (AB019622) starch synthase (GBSS . . . | 921 | 0.0 |
| gi\|136765\|sp\|P27736\|UGST WHEAT | Granule-bound glycogen [star . . . | 915 | 0.0 |
| gi\|4588609\|gb\|AAD26156.1\|AF113844_1 | (AF113844) granule-boun . . . | 910 | 0.0 |
| gi\|4588607\|gb\|AAD26155.1\|AF113843_1 | (AF113843) granule-boun . . . | 889 | 0.0 |
| gi\|6136121\|sp\|O82627\|UGST ANTMA | Granule-bound glycogen [sta . . . | 773 | 0.0 |
| gi\|15637079\|dbj\|BAB68126.1\| | (AB071604) granule-bound starch . . . | 771 | 0.0 |
| gi\|3832512\|gb\|AAC70779.1\| | (AF097922) granule-bound glycogen . . . | 765 | 0.0 |
| gi\|2833381\|sp\|Q42857\|UGST IPOBA | Granule-bound glycogen [sta . . . | 764 | 0.0 |
| gi\|12003285\|gb\|AAG43519.1\|AF210699_1 | (AF210699) granule-bou . . . | 762 | 0.0 |
| gi\|228210\|prf\|\|1718316A | granule-bound starch synthase [Sola . . . | 758 | 0.0 |
| gi\|15626365\|emb\|CAC69955.1\| | (AJ345045) granule-bound starch . . . | 756 | 0.0 |
| gi\|267196\|sp\|Q00775\|UGST SOLTU | Granule-bound glycogen [star . . . | 754 | 0.0 |
| gi\|2833388\|sp\|Q43784\|UGST MANES | Granule-bound glycogen [sta . . . | 754 | 0.0 |
| gi\|602594\|emb\|CAA58220.1\| | (X83220) starch (bacterial glycog . . . | 752 | 0.0 |
| gi\|15223331\|ref\|NP 174566.1\| | (NM_103023) starch synthase, p . . . | 742 | 0.0 |
| gi\|5441242\|dbj\|BAA82346.1\| | (AB029546) granule-bound starch . . . | 734 | 0.0 |
| gi\|18139611\|gb\|AAL58572.1\| | (AY069940) granule binding starc . . . | 729 | 0.0 |
| gi\|2833383\|sp\|Q43092\|UGST PEA | Granule-bound glycogen [starc . . . | 726 | 0.0 |
| gi\|6492245\|gb\|AAF14233.1\|AF109395_1 | (AF109395) granule-boun . . . | 716 | 0.0 |
| gi\|9587307\|gb\|AAF89255.1\|AF285980_1 | (AF285980) granule-boun . . . | 518 | e−146 |
| gi\|9587337\|gb\|AAF89270.1\|AF285995_1 | (AF285995) granule-boun . . . | 517 | e−145 |
| gi\|9587327\|gb\|AAF89265.1\|AF285990_1 | (AF285990) granule-boun . . . | 514 | e−145 |
| gi\|9587333\|gb\|AAF89268.1\|AF285993_1 | (AF285993) granule-boun . . . | 514 | e−144 |
| gi\|15054922\|gb\|AAK82769.1\|AF292500_1 | (AF292500) granule-bou . . . | 513 | e−144 |
| gi\|9587329\|gb\|AAF89266.1\|AF285991_1 | (AF285991) granule-boun . . . | 513 | e−144 |
| gi\|15054954\|gb\|AAK82785.1\|AF292516_1 | (AF292516) granule-bou . . . | 513 | e−144 |
| gi\|15054970\|gb\|AAK82793.1\|AF292524_1 | (AF292524) granule-bou . . . | 512 | e−144 |
| gi\|15054956\|gb\|AAK82786.1\|AF292517_1 | (AF292517) granule-bou . . . | 512 | e−144 |
| gi\|15054960\|gb\|AAK82788.1\|AF292519_1 | (AF292519) granule-bou . . . | 512 | e−144 |
| gi\|9587325\|gb\|AAF89264.1\|AF285989_1 | (AF285989) granule-boun . . . | 511 | e−144 |
| gi\|3493047\|gb\|AAD02981.1\| | (AF079261) granule-bound starch s . . . | 511 | e−144 |
| gi\|15054930\|gb\|AAK82773.1\|AF292504_1 | (AF292504) granule-bou . . . | 511 | e−144 |
| gi\|9587313\|gb\|AFF89258.1\|AF285983_1 | (AF285983) granule-boun . . . | 509 | e−143 |
| gi\|9587352\|gb\|AAF89276.1\|AF286003_1 | (AF286003) granule-boun . . . | 509 | e−143 |
| gi\|15054968\|gb\|AAK82792.1\|AF292523_1 | (AF292523) granule-bou . . . | 509 | e−143 |
| gi\|9587331\|gb\|AAF89267.1\|AF285992_1 | (AF285992) granule-boun . . . | 509 | e−143 |
| gi\|9587323\|gb\|AAF89263.1\|AF285988_1 | (AF285988) granule-boun . . . | 509 | e−143 |
| gi\|9587299\|gb\|AAF89251.1\|AF285976_1 | (AF285976) granule-boun . . . | 507 | e−142 |
| gi\|15054940\|gb\|AAK82778.1\|AF292509_1 | (AF292509) granule-bou . . . | 507 | e−142 |
| gi\|3493043\|gb\|AAD02979.1\| | (AF079259) granule-bound starch s . . . | 506 | e−142 |
| gi\|9587305\|gb\|AAF89254.1\|AF285979_1 | (AF285979) granule-boun . . . | 506 | e−142 |
| gi\|3493045\|gb\|AAD02980.1\| | (AF079260) granule-bound starch s . . . | 505 | e−142 |
| gi\|9587319\|gb\|AAF89261.1\|AF285986_1 | (AF285986) granule-boun . . . | 504 | e−142 |
| gi\|9587311\|gb\|AAF89257.1\|AF285982_1 | (AF285982) granule-boun . . . | 504 | e−141 |
| gi\|9587343\|gb\|AAF89273.1\|AF285998_1 | (AF285998) granule-boun . . . | 503 | e−141 |
| gi\|9587297\|gb\|AAF89250.1\|AF285975_1 | (AF285975) granule-boun . . . | 503 | e−141 |
| gi\|15054984\|gb\|AAK82800.1\|AF292531_1 | (AF292531) granule-bou . . . | 503 | e−141 |
| gi\|9587339\|gb\|AAF89271.1\|AF285996_1 | (AF285996) granule-boun . . . | 502 | e−141 |
| gi\|9587303\|gb\|AAF89253.1\|AF285978_1 | (AF285978) granule-boun . . . | 501 | e−141 |
| gi\|15054990\|gb\|AAK82803.1\|AF292534_1 | (AF292534) granule-bou . . . | 501 | e−141 |
| gi\|9587317\|gb\|AAF89260.1\|AF285985_1 | (AF285985) granule-boun . . . | 500 | e−140 |
| gi\|9587341\|gb\|AAF89272.1\|AF285997_1 | (AF285997) granule-boun . . . | 500 | e−140 |
| gi\|9587348\|gb\|AAF89274.1\| | (AF286001) granule-bound starch s . . . | 499 | e−140 |
| gi\|9587293\|gb\|AAF89248.1\|AF285973_1 | (AF285973) granule-boun . . . | 499 | e−140 |
| gi\|9587335\|gb\|AAF89269.1\|AF285994_1 | (AF285994) granule-boun . . . | 497 | e−139 |
| gi\|9587321\|gb\|AAF89262.1\|AF285987_1 | (AF285987) granule-boun . . . | 497 | e−139 |
| gi\|9587309\|gb\|AAF89256.1\|AF285981_1 | (AF285981) granule-boun . . . | 493 | e−138 |
| gi\|9587301\|gb\|AAF89252.1\|AF285977_1 | (AF285977) granule-boun . . . | 493 | e−138 |
| gi\|9587295\|gb\|AAF89249.1\|AF285974_1 | (AF285974) granule-boun . . . | 492 | e−138 |
| gi\|3493007\|gb\|AAD02961.1\| | (AF079241) granule-bound starch s . . . | 489 | e−137 |
| gi\|3493041\|gb\|AAD02978.1\| | (AF079258) granule-bound starch s . . . | 489 | e−137 |
| gi\|3493019\|gb\|AAD02967.1\| | (AF079247) granule-bound starch s . . . | 488 | e−137 |
| gi\|3493021\|gb\|AAD02968.1\| | (AF079248) granule-bound starch s . . . | 488 | e−137 |
| gi\|3493005\|gb\|AAD02960.1\| | (AF079240) granule-bound starch s . . . | 488 | e−136 |
| gi\|3493035\|gb\|AAD02975.1\| | (AF079255) granule-bound starch s . . . | 487 | e−136 |
| gi\|3492995\|gb\|AAD02955.1\| | (AF079235) granule-bound starch s . . . | 487 | e−136 |
| gi\|3493037\|gb\|AAD02976.1\| | (AF079256) granule-bound starch s . . . | 487 | e−136 |
| gi\|3493025\|gb\|AAD02970.1\| | (AF079250) granule-bound starch s . . . | 486 | e−136 |
| gi\|3493023\|gb\|AAD02969.1\| | (AF079249) granule-bound starch s . . . | 486 | e−136 |
| gi\|16716335\|gb\|AAC17969.3\| | (AF026420) granule-bound starch . . . | 485 | e−136 |
| gi\|3493011\|gb\|AAD02963.1\| | (AF079243) granule-bound starch s . . . | 485 | e−136 |
| gi\|13377473\|gb\|AAK20725.1\| | (AF318769) granule-bound starch . . . | 485 | e−136 |
| gi\|3493013\|gb\|AAD02964.1\| | (AF079244) granule-bound starch s . . . | 483 | e−135 |
| gi\|3493039\|gb\|AAD02977.1\| | (AF079257) granule-bound starch s . . . | 483 | e−135 |
| gi\|3493015\|gb\|AAD02965.1\| | (AF079245) granule-bound starch s . . . | 483 | e−135 |
| gi\|3493031\|gb\|AAD02973.1\| | (AF079253) granule-bound starch s . . . | 482 | e−135 |
| gi\|3493001\|gb\|AAD02958.1\| | (AF079238) granule-bound starch s . . . | 481 | e−135 |
| gi\|3493017\|gb\|AAD02966.1\| | (AF079246) granule-bound starch s . . . | 480 | e−134 |
| gi\|3493009\|gb\|AAD02962.1\| | (AF079242) granule-bound starch s . . . | 479 | e−134 |
| gi\|17432494\|gb\|AAL38185.1\| | (AY062271) granule-bound starch . . . | 479 | e−134 |
| gi\|13377475\|gb\|AAK20726.1\| | (AF318770) granule-bound starch . . . | 479 | e−134 |
| gi\|3493003\|gb\|AAD02959.1\| | (AF079239) granule-bound starch s . . . | 478 | e−133 |
| gi\|3493033\|gb\|AAD02974.1\| | (AF079254) granule-bound starch s . . . | 478 | e−133 |
| gi\|13774486\|gb\|AAK38882.1\| | (AF353520) granule-bound starch . . . | 477 | e−133 |
| gi\|3492999\|gb\|AAD02957.1\| | (AF079237) granule-bound starch s . . . | 475 | e−133 |
| gi\|3493101\|gb\|AAD03008.1\| | (AF079288) granule-bound starch s . . . | 475 | e−133 |
| gi\|3493109\|gb\|AAD03012.1\| | (AF079292) granule-bound starch s . . . | 474 | e−132 |
| gi\|3492997\|gb\|AAD02956.1\| | (AF079236) granule-bound starch s . . . | 474 | e−132 |
| gi\|3493027\|gb\|AAD02971.1\| | (AF079251) granule-bound starch s . . . | 474 | e−132 |
| gi\|3493029\|gb\|AAD02972.1\| | (AF079252) granule-bound starch s . . . | 468 | e−131 |
| gi\|3493107\|gb\|AAD03011.1\| | (AF079291) granule-bound starch s . . . | 468 | e−130 |
| gi\|3493117\|gb\|AAD03016.1\| | (AF079296) granule-bound starch s . . . | 464 | e−129 |
| gi\|12278453\|gb\|AAG48967.1\| | (AY010980) granule-bound starch . . . | 462 | e−129 |
| gi\|12278435\|gb\|AAG48958.1\| | (AY010971) granule-bound starch . . . | 461 | e−129 |
| gi\|12278421\|gb\|AAG48951.1\| | (AY010964) granule-bound starch . . . | 461 | e−129 |
| gi\|12278479\|gb\|AAG48980.1\| | (AY010993) granule-bound starch . . . | 461 | e−129 |
| gi\|12278423\|gb\|AAG48952.1\| | (AY010965) granule-bound starch . . . | 461 | e−128 |
| gi\|12278419\|gb\|AAG48950.1\| | (AY010963) granule-bound starch . . . | 461 | e−128 |
| gi\|3493081\|gb\|AAD02998.1\| | (AF079278) granule-bound starch s . . . | 461 | e−128 |
| gi\|3493071\|gb\|AAD02993.1\| | (AF079273) granule-bound starch s . . . | 460 | e−128 |
| gi\|12278471\|gb\|AAG48976.1\| | (AY010989) granule-bound starch . . . | 460 | e−128 |
| gi\|12278485\|gb\|AAG48983.1\| | (AY010996) granule-bound starch . . . | 460 | e−128 |
| gi\|3493087\|gb\|AAD03001.1\| | (AF079281) granule-bound starch s . . . | 460 | e−128 |
| gi\|12278437\|gb\|AAG48959.1\| | (AY010972) granule-bound starch . . . | 460 | e−128 |
| gi\|12278449\|gb\|AAG48965.1\| | (AY010978) granule-bound starch . . . | 459 | e−128 |
| gi\|12278495\|gb\|AAG48988.1\| | (AY011001) granule-bound starch . . . | 459 | e−128 |
| gi\|12278491\|gb\|AAG48986.1\| | (AY010999) granule-bound starch . . . | 459 | e−128 |
| gi\|13774484\|gb\|AAK38881.1\| | (AF353519) granule-bound starch . . . | 459 | e−128 |
| gi\|3493083\|gb\|AAD02999.1\| | (AF079279) granule-bound starch s . . . | 459 | e−128 |
| gi\|12278473\|gb\|AAG48977.1\| | (AY010990) granule-bound starch . . . | 459 | e−128 |
| gi\|12278481\|gb\|AAG48981.1\| | (AY010994) granule-bound starch . . . | 459 | e−128 |
| gi\|12278413\|gb\|AAG48947.1\| | (AY010960) granule-bound starch . . . | 459 | e−128 |
| gi\|3493075\|gb\|AAD02995.1\| | (AF079275) granule-bound starch s . . . | 458 | e−128 |
| gi\|12278429\|gb\|AAG48955.1\| | (AY010968) granule-bound starch . . . | 458 | e−128 |
| gi\|12278477\|gb\|AAG48979.1\| | (AY010992) granule-bound starch . . . | 458 | e−128 |
| gi\|12278463\|gb\|AAG48972.1\| | (AY010985) granule-bound starch . . . | 458 | e−128 |
| gi\|12278427\|gb\|AAG48954.1\| | (AY010967) granule-bound starch . . . | 458 | e−128 |
| gi\|12278469\|gb\|AAG48975.1\| | (AY010988) granule-bound starch . . . | 458 | e−128 |
| gi\|12278411\|gb\|AAG48946.1\| | (AY010959) granule-bound starch . . . | 458 | e−128 |
| gi\|13194734\|gb\|AAK15529.1\| | (AF331953) granule-bound starch . . . | 457 | e−127 |
| gi\|12278493\|gb\|AAG48987.1\| | (AY011000) granule-bound starch . . . | 457 | e−127 |
| gi\|12278431\|gb\|AAG48956.1\| | (AY010969) granule-bound starch . . . | 457 | e−127 |
| gi\|12278433\|gb\|AAG48957.1\| | (AY010970) granule-bound starch . . . | 457 | e−127 |
| gi\|12278497\|gb\|AAG48989.1\| | (AY011002) granule-bound starch . . . | 457 | e−127 |
| gi\|12278425\|gb\|AAG48953.1\| | (AY010966) granule-bound starch . . . | 457 | e−127 |
| gi\|12278443\|gb\|AAG48962.1\| | (AY010975) granule-bound starch . . . | 456 | e−127 |
| gi\|12278507\|gb\|AAG48994.1\| | (AY011007) granule-bound starch . . . | 456 | e−127 |
| gi\|13774480\|gb\|AAK38879.1\| | (AF353517) granule-bound starch . . . | 456 | e−127 |
| gi\|12278489\|gb\|AAG48985.1\| | (AY010998) granule-bound starch . . . | 456 | e−127 |
| gi\|3493095\|gb\|AAD03005.1\| | (AF079285) granule-bound starch s . . . | 456 | e−127 |
| gi\|12278457\|gb\|AAG48969.1\| | (AY010982) granule-bound starch . . . | 455 | e−127 |
| gi\|12278451\|gb\|AAG48966.1\| | (AY010979) granule-bound starch . . . | 455 | e−127 |
| gi\|3493093\|gb\|AAD03004.1\| | (AF079284) granule-bound starch s . . . | 455 | e−127 |
| gi\|3493085\|gb\|AAD03000.1\| | (AF079280) granule-bound starch s . . . | 455 | e−127 |
| gi\|3493091\|gb\|AAD03003.1\| | (AF079283) granule-bound starch s . . . | 455 | e−127 |
| gi\|13774482\|gb\|AAK38880.1\| | (AF353518) granule-bound starch . . . | 455 | e−127 |
| gi\|3493097\|gb\|AAD03006.1\| | (AF079286) granule-bound starch s . . . | 454 | e−127 |
| gi\|12278514\|gb\|AAG48997.1\| | (AY011011) granule-bound starch . . . | 454 | e−126 |
| gi\|3493111\|gb\|AAD03013.1\| | (AF079293) granule-bound starch s . . . | 454 | e−126 |
| gi\|3493051\|gb\|AAD02983.1\| | (AF079263) granule-bound starch s . . . | 454 | e−126 |
| gi\|3493073\|gb\|AAD02994.1\| | (AF079274) granule-bound starch s . . . | 454 | e−126 |
| gi\|3493113\|gb\|AAD03014.1\| | (AF079294) granule-bound starch s . . . | 454 | e−126 |
| gi\|12278509\|gb\|AAG48995.1\| | (AY011008) granule-bound starch . . . | 454 | e−126 |
| gi\|3493089\|gb\|AAD03002.1\| | (AF079282) granule-bound starch s . . . | 454 | e−126 |
| gi\|12278503\|gb\|AAG48992.1\| | (AY011005) granule-bound starch . . . | 454 | e−126 |
| gi\|3493115\|gb\|AAD03015.1\| | (AF079295) granule-bound starch s . . . | 454 | e−126 |
| gi\|12278487\|gb\|AAG48984.1\| | (AY010997) granule-bound starch . . . | 454 | e−126 |
| gi\|3493119\|gb\|AAD03017.1\| | (AF079297) granule-bound starch s . . . | 454 | e−126 |
| gi\|3493077\|gb\|AAD02996.1\| | (AF079276) granule-bound starch s . . . | 454 | e−126 |
| gi\|3493103\|gb\|AAD03009.1\| | (AF079289) granule-bound starch s . . . | 453 | e−126 |
| gi\|3493099\|gb\|AAD03007.1\| | (AF079287) granule-bound starch s . . . | 453 | e−126 |
| gi\|3493079\|gb\|AAD02997.1\| | (AF079277) granule-bound starch s . . . | 453 | e−126 |
| gi\|12278417\|gb\|AAG48949.1\| | (AY010962) granule-bound starch . . . | 452 | e−126 |
| gi\|3493065\|gb\|AAD02990.1\| | (AF079270) granule-bound starch s . . . | 452 | e−126 |
| gi\|3493067\|gb\|AAD02991.1\| | (AF079271) granule-bound starch s . . . | 452 | e−126 |
| gi\|12278511\|gb\|AAG48996.1\| | (AY011009) granule-bound starch . . . | 452 | e−126 |
| gi\|12278505\|gb\|AAG48993.1\| | (AY011006) granule-bound starch . . . | 452 | e−126 |
| gi\|3493049\|gb\|AAD02982.1\| | (AF079262) granule-bound starch s . . . | 452 | e−126 |
| gi\|12278501\|gb\|AAG48991.1\| | (AY011004) granule-bound starch . . . | 451 | e−126 |
| gi\|12278475\|gb\|AAG48978.1\| | (AY010991) granule-bound starch . . . | 451 | e−125 |
| gi\|12278499\|gb\|AAG48990.1\| | (AY011003) granule-bound starch . . . | 451 | e−125 |
| gi\|3493055\|gb\|AAD02985.1\| | (AF079265) granule-bound starch s . . . | 450 | e−125 |
| gi\|12278415\|gb\|AAG48948.1\| | (AY010961) granule-bound starch . . . | 449 | e−125 |
| gi\|3493069\|gb\|AAD02992.1\| | (AF079272) granule-bound starch s . . . | 448 | e−125 |
| gi\|13774488\|gb\|AAK38883.1\| | (AF353521) granule-bound starch . . . | 448 | e−125 |
| gi\|3493059\|gb\|AAD02987.1\| | (AF079267) granule-bound starch s . . . | 447 | e−124 |
| gi\|3493057\|gb\|AAD02986.1\| | (AF079266) granule-bound starch s . . . | 446 | e−124 |
| gi\|3493053\|gb\|AAD02984.1\| | (AF079264) granule-bound starch s . . . | 445 | e−124 |
| gi\|12278516\|gb\|AAG48998.1\| | (AY011012) granule-bound starch . . . | 444 | e−123 |
| gi\|3493105\|gb\|AAD03010.1\| | (AF079290) granule-bound starch s . . . | 443 | e−123 |
| gi\|3493121\|gb\|AAD03018.1\| | (AF079298) granule-bound starch s . . . | 440 | e−122 |
| gi\|3493063\|gb\|AAD02989.1\| | (AF079269) granule-bound starch s . . . | 440 | e−122 |
| gi\|3493061\|gb\|AAD02988.1\| | (AF079268) granule-bound starch s . . . | 419 | e−116 |
| gi\|17940636\|gb\|AAL49705.1\| | (AF445166) granule-bound starch . . . | 405 | e−112 |
| gi\|17940634\|gb\|AAL49704.1\| | (AF445165) granule-bound starch . . . | 404 | e−111 |
| gi\|17940622\|gb\|AAL49698.1\| | (AF445159) granule-bound starch . . . | 404 | e−111 |
| gi\|13375389\|gb\|AAK20309.1\| | (AF329738) granule bound starch . . . | 404 | e−111 |
| gi\|17940642\|gb\|AAL49707.1\| | (AF445170) granule-bound starch . . . | 404 | e−111 |
| gi\|17940630\|gb\|AAL49702.1\| | (AF445163) granule-bound starch . . . | 404 | e−111 |
| gi\|17940626\|gb\|AAL49700.1\| | (AF445161) granule-bound starch . . . | 403 | e−111 |
| gi\|17940659\|gb\|AAL49715.1\| | (AF445179) granule-bound starch . . . | 403 | e−111 |
| gi\|17940644\|gb\|AAL49708.1\| | (AF445171) granule-bound starch . . . | 403 | e−111 |
| gi\|17940651\|gb\|AAL49711.1\| | (AF445175) granule-bound starch . . . | 402 | e−111 |
| gi\|13375385\|gb\|AAK20307.1\| | (AF329736) granule bound starch . . . | 402 | e−111 |
| gi\|17940677\|gb\|AAL49723.1\| | (AF445189) granule-bound starch . . . | 402 | e−111 |
| gi\|13375387\|gb\|AAK20308.1\| | (AF329737) granule bound starch . . . | 402 | e−111 |
| gi\|17940655\|gb\|AAL49713.1\| | (AF445177) granule-bound starch . . . | 402 | e−111 |
| gi\|17940667\|gb\|AAL49719.1\| | (AF445183) granule-bound starch . . . | 402 | e−111 |
| gi\|13375399\|gb\|AAK20314.1\| | (AF329743) granule bound starch . . . | 402 | e−111 |
| gi\|17940628\|gb\|AAL49701.1\| | (AF445162) granule-bound starch . . . | 401 | e−111 |
| gi\|17940639\|gb\|AAL49706.1\| | (AF445168) granule-bound starch . . . | 401 | e−110 |
| gi\|17940679\|gb\|AAL49724.1\| | (AF445190) granule-bound starch . . . | 401 | e−110 |
| gi\|13375381\|gb\|AAK20305.1\| | (AF329734) granule bound starch . . . | 400 | e−110 |
| gi\|17940675\|gb\|AAL49722.1\| | (AF445188) granule-bound starch . . . | 400 | e−110 |
| gi\|17940624\|gb\|AAL49699.1\| | (AF445160) granule-bound starch . . . | 400 | e−110 |
| gi\|17940648\|gb\|AAL49710.1\| | (AF445173) granule-bound starch . . . | 400 | e−110 |
| gi\|17940620\|gb\|AAL49697.1\| | (AF445158) granule-bound starch . . . | 400 | e−110 |
| gi\|17940672\|gb\|AAL49721.1\| | (AF445186) granule-bound starch . . . | 400 | e−110 |
| gi\|17940653\|gb\|AAL49712.1\| | (AF445176) granule-bound starch . . . | 399 | e−110 |
| gi\|17940632\|gb\|AAL49703.1\| | (AF445164) granule-bound starch . . . | 399 | e−110 |
| gi\|17940661\|gb\|AAL49716.1\| | (AF445180) granule-bound starch . . . | 398 | e−110 |
| gi\|15983795\|gb\|AAL10494.1\| | (AY056803) At1g32900/F9L11_8 [Ar . . . | 386 | e−106 |
| gi\|553108\|gb\|AAA33918.1\| | (M55039) UDP-glucose starch glycos . . . | 379 | e−104 |
| gi\|13375405\|gb\|AAK20317.1\| | (AF329746) granule-bound starch . . . | 378 | e−103 |
| gi\|8778105\|gb\|AAF79205.1\|AF267643_1 | (AF267643) starch synth . . . | 377 | e−103 |
| gi\|6116748\|dbj\|BAA85761.1\| | (AB028026) granule-bound starch . . . | 377 | e−103 |
| gi\|1136128\|gb\|AAA84384.1\| | (U41446) starch synthase [Sorghum . . . | 355 | 7e−97 |
| gi\|7433860\|pir\|\|T07802 | ADPglucose - starch glucosyltransfera . . . | 329 | 5e−89 |
| gi\|7433861\|pir\|\|T07804 | ADPglucose - starch glucosyltransfera . . . | 328 | 1e−88 |
| gi\|2829792\|sp\|P93568\|UGS2_SOLTU | Soluble glycogen [starch] s . . . | 320 | 4e−86 |
| gi\|2833384\|sp\|Q43093\|UGS3_PEA | Glycogen [starch] synthase, c . . . | 314 | 2e−84 |
| gi\|2129898\|pir\|\|S61505 | UDPglucose - starch glucosyltransfera . . . | 314 | 2e−84 |
| gi\|7489695\|pir\|\|T06798 | probable starch synthase (EC 2.4.1. - . . . | 313 | 2e−84 |
| gi\|8708896\|gb\|AAC17970.2\| | (AF026421) soluble starch synthas . . . | 312 | 5e−84 |
| gi\|9369336\|emb\|CAB99210.1\| | (AJ292522) starch synthase I-2 [. . . | 311 | 2e−83 |
| gi\|6103327\|gb\|AAF03557.1\| | (AF091802) starch synthase I [Aeg . . . | 311 | 2e−83 |
| gi\|15237934\|ref\|NP_197818.1\| | (NM_122336) soluble starch syn . . . | 310 | 2e−83 |
| gi\|5880466\|gb\|AAD54661.1\| | (AF091803) starch synthase I [Tri . . . | 310 | 2e−83 |
| gi\|9369334\|emb\|CAB99209.1\| | (AJ292521) starch synthase I-1 [. . . | 310 | 3e−83 |
| gi\|7188796\|gb\|AAF37876.1\|AF234163_1 | (AF234163) starch synth . . . | 310 | 4e−83 |
| gi\|15232051\|ref\|NP_186767.1\| | (NM_110984) putative glycogen . . . | 309 | 6e−83 |
| gi\|6690399\|gb\|AAF24126.1\|AF121673_1 | (AF121673) soluble star . . . | 308 | 1e−82 |
| gi\|7489710\|pir\|\|T01208 | ADPglucose - starch glucosyltransfera . . . | 308 | 1e−82 |
| gi\|3192881\|gb\|AAC19119.1\| | (AF068834) starch synthase [Ipomo . . . | 307 | 3e−82 |
| gi\|16265834\|gb\|AAL16661.1\|AF419099_1 | (AF419099) putative so . . . | 304 | 2e−81 |
| gi\|2833387\|sp\|Q43654\|UGS2_WHEAT | Soluble glycogen [starch] s . . . | 302 | 5e−81 |
| gi\|7489712\|pir\|\|T01414 | ADPglucose--starch glucosyltransfera . . . | 300 | 2e−80 |
| gi\|2833377\|sp\|Q40739\|UGS2_ORYSA | Soluble glycogen [starch] s . . . | 300 | 3e−80 |
| gi\|1549232\|dbj\|BAA07396.1\| | (D38221) SSS1 [Oryza sativa] >gi . . . | 300 | 3e−80 |
| gi\|12019656\|gb\|AAD45815.2\| | (AF168786) soluble starch syntha . . . | 300 | 3e−80 |

SSI
Amino acid sequence of maize Soluble Starch Synthase I (SSI)

```
Accession: AF036891
                      NID: g2828011
                      Mol. wt. (calc) = 67843 Residues = 622
```

| | | |
|---|---|---|
| 1 | M A T P S A V G A A C L L L A R A A W P A A V G D R A R P R | SEQ. ID. No. 267 |
| 31 | R L Q R V L R R R C V A E L S R E G P A P R P L P P A L L A | |
| 61 | P P L V P G F L A P P A E P T G E P A S T P P P V P D A G L | |
| 91 | G D L G L E P E G I A E G S I D N T V V V A S E Q D S E I V | |
| 121 | V G K E Q A R A K V T Q S I V F V T G E A S P Y A K S G G L | |
| 151 | G D V C G S L P V A L A A R G H R V M V V M P R Y L N G T S | |
| 181 | D K N Y A N A F Y T E K H I R I P C F G G E H E V T F F H E | |
| 211 | Y R D S V D W V F V D H P S Y H R P G N L Y G D K F G A F G | |
| 241 | D N Q F R Y T L L C Y A A C E A P L I L E L G G Y I Y G Q N | |
| 271 | C M F V V N D W H A S L V P V L L A A K Y R P Y G V Y K D S | |
| 301 | R S I L V I H N L A H Q G V E P A S T Y P D L G L P P E W Y | |
| 331 | G A L E W V F P E W A R R H A L D K G E A V N F L K G A V V | |
| 361 | T A D R I V T V S K G Y S W E V T T A E G G Q G L N E L L S | |
| 391 | S R K S V L N G I V N G I D I N D W N P A T D K C I F C H Y | |
| 421 | S V D D L S G K A K C K G A L Q K E L G L P I R P D V P L I | |
| 451 | G F I G R L D Y Q K G I D L I Q L I I P D L M R E D V Q F V | |
| 481 | M L G S G D P E L E D W M R S T E S I F K D K F R G W V G F | |
| 511 | S V P V S H R I T A G C D I L L M P S R F E F C G L N Q L Y | |
| 541 | A M Q Y G T V P V V H A T G G L R D T V E N F N P F G E N G | |
| 571 | E Q G T G W A F A P L T T E N M F V D I A N C N I Y I Q G T | |
| 601 | Q V L L G R A N EA R H V K R L H V G P C R | |

TABLE XIII
Maize soluble starch synthase I (SSI)
Alignments with other similar proteins—Transit Peptide

| SEQ | Accession | a.a | | a.a. |
|---|---|---|---|---|
| Id.No. | Number | # | (start) Sequence ending | # |
| 268 | MAIZESSI | 1 | MATPSAVGAACLLLARAA-----WPAAVGDXXXXXXXXXXXXXXCVAELSREG--XXXXX | 53 |
| 269 | 7489712 | 1 | MATPSAVGAACLLLARAA-----WPAAVGDRARPRRLQRVLRRRCVAELSREG--PAPRP | 53 |
| 270 | 12019656 | 1 | MATPSAVGAACLVLARAAAGLGLGPGRGGDRARPRRFQRVVRRRCVAELSREGPAPTPRP | 60 |
| 271 | 1549232 | 34 | CVAELSRDG--GSAQR | 47 |
| 272 | 2833377 | 34 | CVAELSRDG--GSAHG | 47 |
| 273 | 5295947 | 34 | CVAELSRDG--GSAQR | 47 |
| 268 | MaizeSSI | 54 | XXXXXXXXXXXXXXXXXXXEPTGEPASTPPPVP--------------DAGLGDLG--LEP | 97 |
| 269 | 7489712 | 54 | LPPALLAPPLVPGFLAPPAEPTGEPASTPPPVP--------------DAGLGDLG--LEP | 97 |
| 270 | 12019656 | 61 | LPPALLAPPLVPAFLAPPSEPEGEPASTFPPLP--------------DAGLGDLG--LQP | 104 |
| 271 | 1549232 | 48 | PLAPAPLVKQPVL-------PTFLVPTSTPPAPTQSPAPAPTPPPLPDSGVGEIE--PDL | 98 |
| 272 | 2833377 | 48 | PLAPAPLVKQPVL-------PTFLVPTSTPPAPTQSPAPAPTPPPLPDSGVGEIE--PDL | 98 |
| 273 | 5295947 | 48 | PLAPAPLVKQPVL-------PTFLVPTSTPPAPTQSPAPAPTPPPLPDSGVGEIE--PDL | 98 |
| 274 | 9369336 | 79 | PAQSPAPTQPPLP--------------DAGVGELAPDLLL | 104 |
| 275 | 6103327 | 79 | PAQSPAPTQPPLP--------------DAGVGELAPDLLL | 104 |
| 276 | 9369334 | 79 | PAQSPAPTQPPLP--------------DAGVGELAPDLLL | 104 |
| 277 | 5880466 | 79 | PAQSPAPTQPPLP--------------DAGVGELAPDLLL | 104 |
| 278 | 7188796 | 75 | PAQSPAPTQPPLP--------------DAGVGELAPDLLL | 100 |
| 279 | 16265834 | 281 | EP | 282 |
| 280 | 2833381 | 25 | GLGQLA--LRS | 33 |
| 281 | 297424 | 31 | GFQG--LKP | 37 |
| 282 | 82478 | 31 | GFQG--LKP | 37 |
| 283 | 2833382 | 31 | GFQG--LKP | 37 |

TABLE XIV
Maize soluble starch synthase I (SSI)
“GLASS” Domain Alignments with other similar proteins

| SEQ | Accession | a.a | | a.a. |
|---|---|---|---|---|
| Id.No. | Number | # | (start) Sequence ending | # |
| 284 | MaizeSSI | 98 | EGIAEG-----SID--NTVVVASEQD--SEIVVGKEQA--R-----AKVT---------- | 131 |
| 285 | 7489712 | 98 | EGIAEG-----SID--NTVVVASEQD--SEIVVCKEQA--R-----AKVT---------- | 131 |
| 287 | 12019656 | 105 | EGIAEG-----SID--ETVVVASEQD--SEIVVGKEQA--R-----AKVT---------- | 138 |
| 288 | 1549232 | 99 | EGLTED-----SID--KTIFVASEQE--SEIMDVKEQA--Q-----AKVT---------- | 132 |
| 289 | 2833377 | 99 | EGLTED-----SID--KTIFVASEQE--SEIMDVKEQA--Q-----AKVT---------- | 132 |
| 290 | 5295947 | 99 | EGLTED-----SID--KTIFVASEQE--SEIMDVKEQA--Q-----AKVT---------- | 132 |
| 291 | 9369336 | 105 | EGIAED-----SID--SIIVAASEQD--SEIMDAKDQP--Q-----AKVT---------- | 138 |
| 292 | 6103327 | 105 | EGIAED-----SID--SIIVAASEQD--SEIMDANEQP--Q-----AKVT---------- | 138 |
| 293 | 9369334 | 105 | EGIAED-----SID--SIIVAASEQD--SEIMDANEQP--Q-----AKVT---------- | 138 |
| 294 | 5880466 | 105 | EGIAED-----SID--SIIVAASEQD--SEIMDANEQP--Q-----AKVT---------- | 138 |
| 295 | 7188796 | 101 | EGIAED-----SID--TIVVAASEQD--SEIMDANDQP--L-----AKVT---------- | 134 |
| 296 | 15237934 | 131 | VGIPSG--K-----AEVV---------- | 141 |
| 297 | 6690399 | 54 | VGIPSG--K-----AEVV---------- | 64 |
| 298 | 2829792 | 102 | VE--SHDIVANDRDDLSEDTEEMEET--P-----IKLT---------- | 130 |
| 299 | 15232051 | 268 | EK-----YVE--KTPDVASSET--NE--PGKDEE--KPPPLAGANV---------- | 300 |
| 300 | 8708896 | 65 | AEEEE--PPAVEKPPPL--A-----GPNV---------- | 84 |
| 301 | 15384987 | 126 | VSSADD--SENKESGPLA--G-----PNVM---------- | 146 |
| 302 | 15028467 | 183 | VSSADD--SENKESGPLA--G-----PNVM---------- | 203 |
| 303 | 16265834 | 283 | DAAEDG-----DDD--DDWADSDASD--SEIDQDDDSGPLA-----GENV---------- | 318 |
| 304 | 5441242 | 68 | GREKV--L-----EKIECG-------- | 79 |
| 305 | 12003285 | 54 | --NNVKVSAKRV--GQVPINKDRC--E-----TS-G---------- | 77 |
| 306 | 2833381 | 34 | QAVTHN-----GLRPVNKIDMLQLRT--SAPNLAKMEG--K-----MRVEWQAGTIVCKQ | 79 |
| 307 | 297424 | 38 | RSPAGGDATSLSVT--TSARATPKQQ--RSVQRGSRRF--P-----SVVVYATGAG---- | 82 |
| 308 | 82478 | 38 | RSPAGGDATSLSVT--TSARATPKQQ--RSVQRGSRRF--P-----SVVVYATGAG---- | 82 |
| 309 | 2833385 | 78 | -----ATAG---------- | 81 |
| 310 | 2833382 | 38 | RSPAGGDATSLSVT--TSARATPKQQ--RSVQRGSRRF--P-----SVVVYATGAG---- | 82 |
| 284 | (maize SSI) | 132 | --QSIVFVTGEASPYAKSGGLGDVCGSLPVALAARGHRVMVVMPRYLN-GT-SDKNYANA | 187 |
| 285 | 7489712 | 132 | --QSIVFVTGEASPYAKSGGLGDVCGSLPVALAARGHRVMVVMPRYLN-GT-SDKNYANA | 187 |
| 286 | 12019656 | 139 | --QSIVFVTGEASPYAKSGGLGDVCGSLPVALAARGHRVMVVMPRYLN-GT-SDKNYANA | 194 |
| 287 | 1549232 | 133 | --RSVVFVTGEASPYAKSGGLGDVCGSLPIALALRGNRVMVVMPRYMN-GA-LNKNFANA | 188 |
| 288 | 2833377 | 133 | --RSVVFVTGEASPYAKSGGLGDVCGSLPIALAIRGHRVMVVMPRYMN-GA-LNKNFANA | 188 |
| 289 | 5295947 | 133 | --RSVVFVTGEASPYAKSGGLGDVCGSLPIALALRGHRVMVVMPRYMN-GA-LNKNFANA | 188 |
| 290 | 9369336 | 139 | --RSIVFVTGEAAPYAKSGGLGDVCGSLPIALAARGHRVMVVMPRYLN-GS-SDKNYAKA | 194 |
| 291 | 6103327 | 139 | --RSIVFVTGEAAPYAKSGGLGDVCGSLPIALAARGHRVMVVMPRYLN-GS-SDKNYAKA | 194 |
| 292 | 9369334 | 139 | --RSIVFVTGEAAPYAKSGGLGDVCGSLPIALAARGHRVMVVMPRYLN-GS-SDKNYAKA | 194 |
| 293 | 5880466 | 139 | --RSIVFVTGEAAPYAKSGGLGDVCGSLPIALAARGHRVMVVMPRYLN-GS-SDKNYAKA | 194 |
| 294 | 7188796 | 135 | --RSIVFVTGEAAPYAKSGGLGDVCGSLPIALAARGHRVMVVMPRYLN-GT-SDKNYAKA | 190 |
| 295 | 2833387 | 1 | -- EAAPYAKSGGLGDVCGSLPIALAARGHRVMVVMPRYLN-GS-SDKNYAKA | 48 |
| 296 | 15237934 | 142 | --NNLVFVTSEAAPYSKTGGLGDVCGSLPIALAGRGHRVMVISPRYLN-GTAADKNYARA | 198 |
| 297 | 6690399 | 65 | --NNLVFVTSEAAPYSKTGGLGDVCGSLPIALAGRGHRVMVISPRYLN-GTAADKNYARA | 121 |
| 298 | 2829792 | 131 | --FNIIFVTAEAAPYSKTGGLGDVCGSLPMALAARGHRVMVVSPRYLNGGP-SDEKYANA | 187 |
| 299 | 15232051 | 301 | --MNVILVAAECAPFSKTGGLGDVAGALPKSLARRGHRVMVVVPRY--------AEYAEA | 350 |
| 311 | 3192881 | 140 | NVILVCAECAPWSKTGGLGDVAGALPKALARRGHRVMVVVPLY--------GNYAEP | 188 |
| 310 | 2833384 | 262 | NIILVSAECAPWSKTCGLGDVAGSLPKALARRGHRVMIVAPHY--------GNYAEA | 310 |
| 312 | 2129898 | 262 | NIILVSAECAPWSKTGGLGDVAGSLPKALARRGHRVMIVAPHY--------GNYAEA | 310 |
| 313 | 6467503 | 261 | NVILVAAECAPWSKTGGLGDVAGSLPKALAPRGHRVMVVAPRY--------GNYVEP | 309 |
| 314 | 14495348 | 259 | NIILVAAECAPWSKTGGLGDVAGALPKALARRGHRVMVVVPMY--------KNYAEP | 307 |
| 315 | 7489711 | 208 | NVVVVASECAPFCKTGGLGDVVGALPKALARRGHRVMVVIPRY--------GEYAEA | 256 |
| 300 | 8708896 | 85 | --NNVVMVGAECAPWSKTGGLGDVMAALPKALVRRGHRVMVVVPRY--------ENYDNA | 134 |
| 301 | 15384987 | 147 | --NVIV-VASECSPFCKTGGLGDVVGALPKALARRGHRVMVVIPRY--------GEYAEA | 195 |
| 302 | 15028467 | 204 | --NVIV-VASECSPFCKTGGLGDVVGALPKALARRGHRVMVVIPRY--------GEYAEA | 252 |
| 316 | 7489710 | 242 | NVIVVAAECSPWCKTGGLGDVVGALPKALARRGHRVMVVVPRY--------GDYVEA | 290 |
| 317 | 2833390 | 297 | NIILVASECAPWSKTGGLGDVAGALPKALARRGHRVMVVAPRYDN-YP-EPQDSG-- | 349 |
| 318 | 7489695 | 1 | NVVVVAAECSPWCKTGGLGDVAGALPKALAKRGHRVMVVVPRY--------GDYEEA | 49 |
| 319 | 8953573 | 308 | NVVVVAAECSPWCKTGGLGDVAGALPKALAKRGHRVMVVVPRY--------GDYEEA | 356 |
| 320 | 8953571 | 309 | NVVVVAAECSPWCKTGGLGDVAGALPKALAKRGHRVMVVVPRY--------GDYEEA | 357 |
| 321 | 7529653 | 309 | NVVVVAAECSPWCKTGGLGDVAGALPKALAKRGHRVMVVVPRY--------GDYEEA | 357 |
| 303 | 16265834 | 319 | --MNVIVVAAECSPWCKTGGLGDVAGALPKALARRGHRVMVVVPRY--------GDYAEA | 368 |
| 322 | 5825480 | 309 | NVVVVAAECSPWCKTGGLGDVAGALPKALAKRGHRVMVVVPRY--------GDYEEA | 357 |
| 323 | 2833388 | 83 | NLIFVGAEVGPWSKTGGLGDVLGGLPRAMAARGHRVMTVSPRY--------DQYKDA | 131 |
| 304 | 5441242 | 80 | --MNLIFVGAEVAPWSKTGGLGDVLGGLPSALAEHGHRVMTVSPRY--------DQYKDA | 129 |
| 305 | 12003285 | 78 | --MTLIFVSAECGPWSKTGGLGDVVGGLPPALAANRHRVMTVSPRY--------DQYKDA | 127 |
| 324 | 267196 | 82 | NLIFVGTEVGPWSKTGGLGDVLGGLPPALAARGHRVMTISPRY--------DQYKDA | 130 |
| 325 | 228210 | 82 | NLIFVGTEVGPWSKTGGLGDVLGGLPPALAARGHRVMTISPRY--------DQYKDA | 130 |
| 326 | 602594 | 82 | NLIFVGTEVGPWSKTGGLGDVLGGLPPALAARGURVMTISPRY--------DQYKDA | 130 |
| 327 | 16716335 | 61 | IVMVAAEVAPWSKTGGLGDVTGGLPIELVKRGHRVMTIAPRY--------DQYADA | 108 |
| 328 | 6136121 | 83 | NLVFVLAEVGPWSKTGGLGDVVGGLPPAMAGNGHRVMTVSPRY--------DQYKDA | 131 |
| 329 | 15223331 | 85 | SVIFIGAEVGPWSKTGGLGDVLGGLPPALAARGHRVMTICPRY--------DQYKDA | 133 |
| 330 | 15626365 | 88 | NLIFVGTEVAPWSKTGGLGDVLGGLPPALSANGHRVMTVTPRY--------DQYKDA | 136 |
| 331 | 3832512 | 82 | NLVFVGAEVGPWSKTGGLGDVLGGLPPALAGNGHRVMTVSPRY--------DQYKDA | 130 |
| 332 | 2833381 | 80 | QGMNLVFVGCEEGPWCKTGGLGDVLGGLPPAIAARGHRVMTVCPRY--------DQYKDA | 131 |
| 333 | 15637079 | 83 | NLVFVGCEVGPWCKTGGLGDVLGGLPPALAARGHRVMTVCPRY--------DQYKDA | 131 |
| 334 | 2833383 | 78 | SLVFVGAEVGPWSKTGGLGDVLGGLPPVLAGNGHRVMTVSPRY--------DQYKDA | 126 |
| 307 | 297424 | 83 | --MNVVFVGAEMAPWSKTGGLGDVLGGLPPAMAANGHRVMVISPRY--------DQYKDA | 132 |
| 335 | 82478 | 83 | --MNVVFVGAEMAPWSKTGGLGDVLGGLPPAMAANGHRVMVISPRY--------DQYKDA | 132 |
| 309 | 2833385 | 82 | --MNVVFVGAEMAPWSKTGGLGDVLGGLPPAMAANGHRVMVVSPRY--------DQYKDA | 131 |
| 336 | 2833382 | 83 | --MNVVFVGAEMAPWSKTGGLGDVLGGLPPAMAANGHRVMVISPRY--------DQYKDA | 132 |
| 284 | (maize SSI) | 188 | FYTEKHIRIPCFGGEHE-VTFFHEYRDSVDWVFV-DHPSY------H-R-P------G-N | 230 |
| 285 | 7489712 | 188 | FYTEKHIRIPCFGGEHE-VTFFHEYRDSVDWVFV-DHPSY------N-R-P------G-N | 230 |
| 286 | 12019656 | 195 | FYTEKHIRIFCFGGEHE-VTFFHEYRDSVDWVFV-DHPSY------H-R-P------G-N | 237 |
| 287 | 1549232 | 189 | FYTEKHIKIPCFGGEHE-VTFFHEYRDSVDWVFV-DHPSY------H-R-P------G-N | 231 |
| 288 | 2833377 | 189 | FYTEKHIKIPCFGGEHE-VTFFHEYRDSVDWVFV-DHPSY------H-R-P------G-N | 231 |
| 289 | 5295947 | 189 | FYTEKHIKIPCFGGEHE-VTFFHEYRDSVDWVFV-DHPSY------H-R-P------G-N | 231 |
| 290 | 9369336 | 195 | LYTAKHIKIPCFGGSHE-VTFFHEYRDNVDNVFV-DHPSY------H-R-P------G-S | 237 |
| 292 | 6103327 | 195 | LYTAKHIKIPCFGGSHE-VTFFHEYRDNVDWVFV-DHPSY------H-R-P------G-S | 237 |
| 292 | 9369334 | 195 | LYTGKHIKIPCFGGSHE-VTFFHEYRDNVDWVFV-DHPSY------H-R-P------G-S | 237 |
| 293 | 5880466 | 195 | LYTGKHIKIPCFGGSHE-VTFFHEYRDNVDWVFV-DHPSY------H-R-P------G-S | 237 |
| 294 | 7188796 | 191 | LYTGKUIKIPCFGGSHE-VTFFHEYRDNVDWVFV-DHPSY------H-R-P------G-S | 233 |
| 295 | 2833387 | 49 | LYTAKHIKIPCFGGSHE-VTFFHEYRDNVDWVFV-DHPSY------H-R-P------G-S | 91 |
| 296 | 15237934 | 199 | KDLGIRVTVNCFGGSQE-VSFYHEYRDGVDWVFV-DHKSY------H-R-P------G-N | 241 |
| 297 | 6690399 | 122 | KDLGIRVTVNCFGGSQE-VSFYHEHRDGVDWVFV-DHKSY------H-R-P------G-N | 164 |
| 298 | 2829792 | 188 | VDLDVRAIVHCFGDAQE-VAFYHEYRAGVDWVFV-DHSSY------C-R-P------G-T | 230 |
| 299 | 15232051 | 351 | KDLGVRKRYKVAGQDME-VMYFHAFIDGVDFVFI-DSPEFR-----H-L-S------N-N | 394 |
| 311 | 3192881 | 189 | QHTGVRKMFKIDGQDME-VNYFHAYIDNVDFVFI-DSPIFQ-----H-R-G------N-N | 232 |
| 310 | 2833384 | 311 | HDIGVRKRYKVAGQDME-VTYFHTYIDGVDIVFI-DSPIF------R-NLE------S-N | 354 |
| 312 | 2129898 | 311 | HDIGVRKRYKVAGQDME-VTYFHTYIDGVDIVFI-DSPIF------R-NLE------S-N | 354 |
| 313 | 6467503 | 310 | QDTGVRKRYKVDGQDFE-VSYFQAFIDGVDFVFI-DSPMF------R-H-I------G-N | 352 |
| 314 | 14495348 | 308 | QQLGEPRRYQVAGQDME-VIYYHAYIDGVDFVFI-DNPIF------H-H-V------E-N | 350 |
| 315 | 7489711 | 257 | RDLGVRRRYKVAGQDSE-VTYFHSYIDGVDFVFV-EAPPFR-----H-R-H------N-N | 300 |
| 300 | 8708896 | 135 | WETGIRKIYSVFNSNQE-VGYFHAFVDGVDYVFV-DHPTF------HGR-G------K-N | 178 |
| 301 | 15384987 | 196 | KDLGVRKRYRVAGQDSE-VSYFHAFIDGVDFVFL-EAPPFR-----H-R-H------N-D | 239 |
| 302 | 15028467 | 253 | KDLGVRKRYRVAGQDSE-VSYFHAFIDGVDFVFL-EAPPFR-----H-R-H------N-D | 296 |
| 316 | 7489710 | 291 | FDMGIRKYYKAAGQDLE-VNYFHAFIDGVDFVFI-DAPLFR-----H-R-Q------D-D | 334 |
| 317 | 2833390 | 350 | --VRKIYKVD--GQDVD-VTYFQALLMDCDFVFIHSHMFR------H-I-G------N-N | 389 |
| 318 | 7489695 | 50 | YDVGVRKYYKAAGQDME-VNYFHAYIDGVDFVFI-DAPLFR-----H-R-Q------E-D | 93 |
| 320 | 8953573 | 357 | YDVGVRKYYKAAGQDME-VNYFHAYIDGVDFVFI-DAPLFR-----H-R-Q------E-D | 400 |
| 320 | 8953571 | 358 | YDVGVRKYYKAAGQDME-VNYFHAYIDGVDFVFI-DAPLFR-----H-R-Q------E-D | 401 |
| 321 | 7529653 | 358 | YDVGVRKYYKAAGQDME-VNYFHAYIDGVDFVFI-DAPLFR-----H-R-Q------E-D | 401 |
| 303 | 16265834 | 369 | QDVGIRKYYKAAGQDLE-VKYFHAFIDGVDFVFI-DAPLFR-----H-R-Q------D-D | 412 |
| 322 | 5825480 | 358 | YDVGVRKYYKAAGQDME-VNYFHAYIDGVDFVFI-DAPLFR-----H-R-Q------E-D | 401 |
| 323 | 2833388 | 132 | WDTSVSVEIKIGDRIET-VRFFHSYKRGVDRVFV-DHPMF------L-E-KVWGKTGS-K | 180 |
| 304 | 5441242 | 130 | WDTNVTVEVKVADRIET-VRFFHCYKQGVDRVFV-DHPCF------L-E-KVWGKTGS-K | 178 |
| 305 | 12003285 | 128 | WDTSVVVEIQVGDKVET-VGFFHCYKRGVDRVFV-DHPLFLEKVWGK-T-K------S-K | 176 |
| 324 | 267196 | 131 | WDTSVAVEVKVGDSIEI-VRFFHCYKRGVDRVFV-DHPMF------L-E-KVWGKTGS-K | 179 |
| 325 | 228210 | 131 | WDTSVAVEVKVGDSIEI-VRFFHCYKRGVDRVFV-DHPMF------L-E-KVWGKTGS-K | 179 |
| 326 | 602594 | 131 | WDTSVAVEVKVGDSIEI-VRFFHCYKRGVDRVFV-DHPMF------L-E-KVWGKTGS-K | 179 |
| 327 | 16716335 | 109 | WDTSVVVDIM---GE-K-VRYFHSIKKGVHRVWI-DHPWFLAKVWGK-T-G------S-K | 153 |
| 328 | 6136121 | 132 | WDTSVVVEIKVGDSIET-VRFFHCYKRGVDRVFV-DHPIFLEKVWGK-T-K------S-K | 180 |
| 329 | 15223331 | 134 | WDTCVVVQIKVGDKVEN-VRFFHCYKRGVDRVFV-DHPIFLAKVVGK-T-G------S-K | 182 |
| 330 | 15626365 | 137 | WDTNVTIEVKVGDRTEK-VRFFHCFKRGVDRVFV-DHPIF------L-E-KVWGKTGT-K | 185 |
| 331 | 3832512 | 131 | WDTGVSVEIKVGDRFET-VRFFHCYKRGVDRVFV-DHPLFLEKVWGK-T-E------S-K | 179 |
| 332 | 2833381 | 132 | WETCVVVE-PQVGDRIEPVRFFHSYKRGVDRVFV-DHPMFLEKVW-G-K-T------G-S | 179 |
| 333 | 15637079 | 132 | WDTCVVVELQVGDRIEP-VRFFHSYKRGVDRVFV-DHPMFLEKVE-G-K-T------G-S | 179 |
| 334 | 2833383 | 127 | WDTNVLVEVKVGDKIET-VRFFHCYKRGVDRVFV-DHPLFLERVWGK-T-G------S-K | 175 |
| 307 | 297424 | 133 | WDTSVVAEIKVADRYER-VRFFHCYKRGVDRVFI-DHPSFLEKVW-G-K-T------GEK | 181 |
| 335 | 82478 | 133 | WDTSVVAEVKVADRYER-VRFFHCYKRGVDRVFI-DHPSFLEKVW-G-K-T------GEK | 181 |
| 309 | 2833385 | 132 | WDTSVVSEIKMGDGYET-VRFFHCYKRGVDRVFI-DHPLFLERVWGK-T-E------E-K | 180 |
| 310 | 2833382 | 133 | WDTSVVAEIKVADRYER-VRFFRCYKRGVDRVFI-DHPSFLEKVW-G-K-T------GEK | 181 |
| 284 | (Maize SSI) | 231 | -LYGDKFG-A-FGDNQFRYTLLCYAACEAPLILEL--GG--Y-----I-----YG-Q-NC | 271 |
| 285 | 7489712 | 231 | -LYGDKFG-A-FGDNQFRYTLLCYAACEAPLILEL--GG--Y-----I-----YG-Q-NC | 271 |
| 286 | 12019656 | 238 | -LYGDKFG-A-FGDNQFRYTLLCYAACEAPLVLEL--GG--Y-----I-----YG-Q-NC | 278 |
| 287 | 1549232 | 232 | -LYGDNFG-A-FGDNQFRYTLLCYAACEAPLILEL--GG--Y-----I-----YG-Q-KC | 272 |
| 288 | 2833377 | 232 | -LYGDNFG-A-FGDNQFRYTLLCYAACEAPLILEL--GG--Y-----I-----YG-Q-KC | 272 |
| 289 | 5295947 | 232 | -LYGDNFG-A-FGDNQFRYTLLCYAACEAPLILEL--GG--Y-----I-----YG-Q-KC | 272 |
| 290 | 9369336 | 238 | -LYGDNFG-A-FGDNQFRYTLLCYAACEAPLILEL--GG--Y-----I-----YG-Q-NC | 278 |
| 291 | 6103327 | 238 | -LYGDNFG-A-FGDNQFRYTLLCYAACEAPLILEL--GG--Y-----I-----YG-Q-NC | 278 |
| 292 | 9369334 | 238 | -LYGDNFG-A-FGDNQFRYTLLCYAACEAPLILEL--GG--Y-----I-----YG-Q-NC | 278 |
| 293 | 5880466 | 238 | -LYGDNFG-A-FGDNQFRYTLLCYAACEAPLILEL--GG--Y-----I-----YG-Q-NC | 278 |
| 294 | 7188796 | 234 | -LYGDNFG-A-FGDNQFRYTLLCYAACEAPLILEL--GG--Y-----I-----YG-Q-SC | 274 |
| 295 | 2833387 | 92 | -LYGDNFG-A-FGDNQFRYTLLCYAACEAPLILEL--GG--Y-----I-----YG-Q-NC | 132 |
| 296 | 15237934 | 242 | -PYGDSKG-A-FGDNQFRFTLLCHAACEAPLVLPL--GG--F-----T-----YG-E-KS | 282 |
| 297 | 6690399 | 165 | -PYGDSKG-A-FGDNQFRFTLLCHAACEAPLVLPL--GG--F-----T-----YG-E-KS | 205 |
| 298 | 2829792 | 231 | -PYGDIYG-A-FGDNQFRFTLLSHAACEAPLVLPL--GG--F-----T-----YG-E-KC | 271 |
| 299 | 15232051 | 395 | -IYG---G-N-RLDILKRMVLFCKAAVEVPWYVPC--GG--V-----C-----YG-DGNL | 433 |
| 311 | 3192881 | 233 | -IYG---G-N-RVDILKRMDLFCKAAIVVPWHVPC--GG--I-----C-----YG-DGNL | 271 |
| 310 | 2833384 | 355 | -IYG---G-N-RLDILRRMVLFCKAAVEVPWHVPC--GG--I-----C-----YG-DGNL | 393 |
| 312 | 2129898 | 355 | -IYG---G-N-RLDILRRMVLFCKAAVEVPWHVPC--GG--I-----C-----YG-DGNL | 393 |
| 313 | 6467503 | 353 | DIYG---G-N-RMDILKRMVLFCKAAVEVPWHVPC--GG--V-----C-----YG-DGNL | 392 |
| 314 | 14495348 | 351 | DIYG---G-D-RTDILKRMVLLCKAAIEVPWYVPC--GG--Y-----C-----YG-DGNL | 390 |
| 315 | 7489711 | 301 | -IYG---G-E-RLDILKRMILFCKAAVEVPWYAPC--GG--T-----V-----YG-DGNL | 339 |
| 300 | 8708896 | 179 | -IYG---G-E-RQEILFRCALLCKAALEAVWHVPC--GG--I-----T-----YGDD-NL | 217 |
| 301 | 15384987 | 240 | -IYG---G-E-RFDVLKRMILFCKAAVEVPWFAPC--GG--S-----I-----YG-DGNL | 278 |
| 302 | 15028467 | 297 | -IYG---G-E-RFDVLKRMILFCKAAVEVPWFAPC--GG--S-----I-----YG-DGNL | 335 |
| 316 | 7489710 | 335 | -IYG---G-S-RQEIMKRMILFCKVAVEVPWHVPC--GG--V-----C-----YG-DGNL | 373 |
| 317 | 2833390 | 390 | -IYG---G-N-RVDILKRMVLFCKAAIEVPWHVPC--GG--V-----C-----YG-DGNL | 428 |
| 318 | 7489695 | 94 | -IYG---G-S-RQEIMKRMVLFCIAVEVPVWHVPC--GG--V-----P-----YG-DGNL | 132 |
| 319 | 8953573 | 401 | -IYG---G-S-RQEIMKPMILFCKAAVEVPWHVPC--GG--V-----P-----YG-DGNL | 439 |
| 320 | 8953571 | 402 | -IYG---G-S-RQEIMKRMILFCKAAVEVPWHVPC--GG--V-----P-----YG-DGNL | 440 |
| 321 | 7529653 | 402 | -IYG---G-S-RQEIMKRMILFCKAAVEVPWHVPC--GG--V-----P-----YG-DGNL | 440 |
| 303 | 16265834 | 413 | -IYG---G-N-RQEIMKRMILFCKAAVEVPWHVPC--GG--V-----P-----YG-DGNL | 451 |
| 322 | 5825480 | 402 | -IYG---G-S-RQEIMKPMILFCKAAVEVPWHVPC--GG--V-----F-----YG-DGNL | 440 |
| 323 | 2833388 | 181 | -IYGPFAG-LDYQDNQLRFSLLCLAALEAPRVLNL--NS--S-----KNFSGPYG-E-EV | 227 |
| 304 | 5441242 | 179 | -LYGPSAG-VDYEDNQLRYSLLCQAALEAPRVLNL--NSNKY-----FSGP--YG-E-DV | 225 |
| 305 | 12003285 | 177 | -VYGPSAG-VDYEDNQLRFSLLSLAALEAFRVLNL--TSNKY-----FSGP--YG-E-DV | 223 |
| 324 | 267196 | 180 | -IYGPKAG-LDYLDNELRFSLLCQAALEAPKVLNLNSSN--Y-----FSGP--YG-E-DV | 226 |
| 325 | 228210 | 180 | -IYGPKAG-LDYLDNELRFSLLCQAALEAPKVLNLNSSN--Y-----FSGP--YG-E-DV | 226 |
| 326 | 602594 | 180 | -IYGPKAG-LDYLDNELRFSLLCQAALEAPKVLNLNSSN--Y-----FSGP--YG-E-DV | 226 |
| 327 | 16716335 | 154 | -LYGPRSG-ADYLDNHKRFALFCKAAIEAARVLPF--GP---------------G-E-DC | 192 |
| 328 | 6136121 | 181 | -IYGPNAG-TDYQDNQLRFSLLCQAALEAPRVLNLTSSK--Y-----FSGP--YG-E-DV | 227 |
| 329 | 15223331 | 183 | -IYGPITG-VDYNDNQLRFSLLCQAALEAPQVLNL--NSSKY-----FSGF--YG-E-DV | 229 |
| 330 | 15626365 | 186 | -LYGPAAGDD-YQDNQLRFSIFCQAALEAARVLNL--KSNKY-----FSGP--YG-E-DV | 232 |
| 331 | 3832512 | 180 | -LYGPKTG-VDYKDNQLRFSLLCQAALEAPRVLNL--NS--N-----KHFSGPYG-E-DV | 226 |
| 332 | 2833381 | 180 | MLYGPKAG-KDYKDNQLRFSLLCQAALEAPRVLNLNSSN--Y-----FSGP--YG-E-DV | 227 |
| 333 | 15637079 | 180 | MLYGPKAG-KDYKDNQLRFSLLCQAALEAPRVLNLNSSN--Y-----FSGP--YG-E-DV | 227 |
| 334 | 2833383 | 176 | -LYGPKTG-IDYRDNQLRFSLLCQAALEAPRVLNL--NSSKY-----FSGP--YG-E-DV | 222 |
| 307 | 297424 | 182 | -IYGPDTG-VDYKDNQMRFSLLCQAALEAPRILNL--NN--NPYFKGT-----YG-E-DV | 228 |
| 335 | 82478 | 182 | -IYGPDTG-VDYKDNQMRFSLLCQAALEAPRILNL--NN--NPYFKGT-----YG-E-DV | 228 |
| 309 | 2833385 | 181 | -KYGPDAG-TDYKDNQLRFSLLCQAALEAPRILSL--NNNPY-----FSGP--YG-E-DV | 227 |
| 310 | 2833382 | 182 | -IYGPDTG-VDYKDNQMRFSLLCQAALEAPRILNL--NN--NPYFKGT-----YG-E-DV | 228 |
| 284 | (maize 881) | 272 | MFVVNDWHASLVPVLLAAKYRPYGVYKDSRSILVIHNLAHQGVEPASTYPDLGLPPEWYG | 331 |
| 285 | 7489712 | 272 | MFVVNDWHASLVPVLLAAKYRPYGVYKDSRSILVIHNLAHQGVEPASTYPDLGLPPEQYG | 331 |
| 286 | 12019656 | 279 | MFVVNDWHASLVPVLLAAKYRPYGVYKDSRSILVIHNLAHQGVEPASTYPDLGLPPEQYG | 338 |
| 287 | 1549232 | 273 | MFVVNDWHASLVPVLLAAKYRPYGVYPDSRSILVIHNLAHQGVEPASTYPDLGLPPEQYG | 332 |
| 288 | 2833377 | 273 | MFVVNDWHASLVPVLLAAKYRPYGVYRDSRSILVIHNLAHQGVEPASTYPDLGLPPEQYG | 332 |
| 289 | 5295947 | 273 | MFVVNDWHASLVPVLLAAKYRPYGVYRDSRSILVIHNLAHQGVEPASTYPDLGLPPEQYG | 332 |
| 290 | 9369336 | 279 | MFVVNDWHASLVPVLLAAKYRPYGVYKDSRSILVIHNLAHQGVEPASTYPDLGLPPEWYG | 338 |
| 291 | 6103327 | 279 | MFVVNDWHASLVPVLLAAKYRPYGVYKDSRSILVIHNLAHQGVEPASTYPDLGLPPEWYG | 338 |
| 292 | 9369334 | 279 | MFVVNDWHASLVPVLLAAKYRPYGVYKDSRSILVIHNLAHQGVEPASTYPDLGLPPEWYG | 338 |
| 293 | 5880466 | 279 | MFVVNDWHASLVPVLLAAKYRPYGVYKDSRSILVIHNLAHQGVEPASTYPDLGLPPEWYG | 338 |
| 294 | 7188796 | 275 | MFVVNDWHASLVPVLLAAKYRPYGVYKDSRSILVIHNLAHQGVEPASTYPDLGLPPEWYG | 334 |
| 295 | 2833387 | 133 | MFVVNDWHASLVPVLLAAKYRPYGVYKDSRSILVIHNLAHQGVEPASTYPDLGLPPEWYG | 192 |
| 296 | 15237934 | 283 | LFLVNDWHAGLVPILLAAKYRPYGVYKDARSILIIHNLAHQGVEPAATYTNLGLPSEWYG | 342 |
| 297 | 6690399 | 206 | LFLVNDWHAGLVPILLAAKYRPYGVYKDARSILIIHNLAHQGVEPAATYTNLGLPSEWYG | 265 |
| 298 | 2829792 | 272 | LFLANDWHAALVPLLLAAKYRPYGVYKDARSIVAIHNIAHQGVEPAVTYNNLGLPPQWYG | 331 |
| 299 | 15232051 | 434 | AFIANDWHTALLPVYLKAYYPRDHGIMDYTRSVLVIHNIAHQGRGPVDFSYVDLPSHYLD | 493 |
| 311 | 3192881 | 272 | VFIANDWHTALLPVYLKAYFRDNGVMKFTRSVLVIHNIAHQGRGPMDDFSIVDLPAQYAD | 331 |
| 310 | 2833384 | 394 | VFIABDWHTALLPVYLKAYYRDHGLMNYTRSVLVIHNIAHQGRGPVEDFNTVDLSGNYLD | 453 |
| 312 | 2129898 | 394 | VFIABDWHTALLPVYLKAYYRDHGLMNYTRSVLVIHNIAHQGRGPVEDFNTVDLSGNYLD | 453 |
| 313 | 6467503 | 393 | AFIANDWHTALLPVYLKAYYRDNGLMQYTRSVLVIHNIAHQGRGPSGDFSYVGLPEHYI- | 451 |
| 314 | 14495348 | 391 | VFLANDWHTALLPVYLKAYYHDNGFMIYARSVLVIHNIAHQGRGPLDDFSYLDLPVDYMD | 450 |
| 315 | 7489711 | 340 | VFIANDWHTALLPVYLKAYYRDNGLMQYARSVLVIHNIAHQGRGPVDDFVNFDLPEHYID | 399 |
| 300 | 8708896 | 218 | CFIANDWHTALLPVYLQAHYRDYGEMTYARCAFVIHNMAHQGRGPFVESEHLELNEE--- | 274 |
| 301 | 15384987 | 279 | VFIANDWHTALLPVYLKAYYRDNGLMQYTRSVLVIHNIAHQGRGPVDDFATMDLPEHYID | 338 |
| 302 | 15028467 | 336 | VFIANDWHTALLPVCLKAYYRDNGLMQYTRSVLVIHNIAHQGRGPVDDFATMDLPEHYID | 395 |
| 316 | 7489710 | 374 | VFIANDWHTALLPVYLKAYYRDHGLMQYTRSVLVIHNIAHQGRGPVDEFPYMDLPEHYLQ | 433 |
| 317 | 2833390 | 429 | VFIANDWHTALLPAYLKAYYRDNGIMNYTRSVLVIHNIAHQGRGPLEDFSYVDLPPHYMD | 488 |
| 318 | 7489695 | 133 | VFIANDWHTALLPVYLKAYYRDHGLMQYTRSIMVIHNIAHQGRGPVDEFPFTELPEHYL- | 191 |
| 319 | 8953573 | 440 | VFIANDWHTALLPVYLKAYYPHGIJMQYTRSIMVIHNIAHQGRGPVDEFPFTELPEHYL- | 498 |
| 320 | 8953571 | 441 | VFIANDWHTALLPVYLKAYYRDHGLMQYTRSIMVIHNIAHQGRGPVDEFPFTELPEHYL- | 499 |
| 321 | 7529653 | 441 | VFIANDWNTALLPVYLKAYYRDHGLMQYTRSIMVIHNIAHQGRGPVDEFPFTELPEHYL- | 499 |
| 303 | 1626583 | 4452 | VFLANDWHTALLPVYLKAYYRDNGMMQYTRSVILVIHNAYQGRGPVDEFPYMELPEHYLD | 511 |
| 322 | 5825480 | 441 | VFIANDWHTALLPVYLKAYYRDHGLMQYTRSIMVIHINAHQGRGPVDEFPFTELPEHYL- | 499 |
| 323 | 2833388 | 228 | AFIANDWHTALLPCYLKAIYQPMGIYKHAKVAFCIHNIAYQGRFAFSDFPRLNLPDKEFS | 287 |
| 304 | 5441242 | 226 | IFVANDWHTALLPCYLKSMYQTRGVYRNTKVAFCIHNISYQGRHPFEDFPLLNLPNEYRS | 285 |
| 305 | 12003285 | 224 | VFVANDWHTAVLPCYLKTIYQPKGIYTNAKVVLCIHNIAYQGRFAFSDFYKLNLPDQLKS | 283 |
| 324 | 267196 | 227 | LFIANDWHTALIFCYLKSMYQSRGIYLNAKVAFCIHNIAYQGRFSFSDFPLLNLPDEFRG | 286 |
| 325 | 228210 | 227 | LFIANDWHTALIFCYLKSMYQSRGIYLNAKVAFCIHNIAYQGRFSFSDFPLLNLPDEFRG | 286 |
| 326 | 602594 | 227 | LFIANDWHTALIFCYLKSMYQSRGIYLNAKVAFCIHNIAYQGRFSFSDFPLLNLPDEFRG | 286 |
| 327 | 16716335 | 193 | VFVANDWHSALVPVLLKDEYQPKGQFTKAKSVLAIHNIAFQGRNWEEAFKDTKLPQAAFD | 252 |
| 328 | 6136121 | 228 | VFVANDWHTALLPCYLKSMYQSKGMYLHAKVAFCIHNIAYQGRFGSSDFCLLNLPDQFKS | 287 |
| 329 | 15223331 | 230 | VFVANDWNTALLPCYLKSMYQSRGVYMNAKVVFCIHNIAYQGRFAFDDYSLLNLPISFKS | 289 |
| 330 | 15626365 | 233 | IFVANDWHTALISCYMKSMYQSIGIFRNAKVVFCIHNIAYQGRFAFTDYSLLNLPDQFKS | 292 |
| 331 | 3832512 | 227 | VFVANDWHTALLPCYLKSLYKSKGIYKSAKVAFCIHNIAYQGRHAFSDLSLLNLPNEFRS | 286 |
| 332 | 2833381 | 228 | VFVANDWHTALLPCYLKTMYQSRGIYMNAKVAFCIHNIAYQGRFAFSDFSLLNLPDEYKG | 287 |
| 333 | 15637079 | 228 | VFVANDWHTALLPCYLKTMYQSRGIYMNAKVAFCIHNIAYQGRFAFSDFSLLNLPDEYKG | 287 |
| 332 | 2833383 | 223 | IFVANDWHSALIPCYLKSMYKSRGLYKNAKVAFCIHNIAYQGRNAFSDFSLLNLPDEFRS | 282 |
| 307 | 297424 | 229 | VFVCNDWHTGPLASYLKNNYQPNGIYFNAKVAFCIHNISYQGRFAFEDYPELNLSERFRS | 288 |
| 335 | 82478 | 229 | VFVCNDWHTGPLASYLKNNYQPNGIYPNAKVAFCIHNISYQGRFAFEDYPELNLSERFRS | 288 |
| 309 | 2833385 | 228 | VFVCNDWHTGPLSCYLKSNYQSNGIYKDAKTAFCIHNISYQGRFAFSDFPELNLPERFKS | 287 |
| 310 | 2833382 | 229 | VFVCNDWHTGPLASYLKSNYQPNGIYRNAKVAFCIHNISYQGRFAFEDYPELNLSERFRS | 288 |

TABLE XV
Maize soluble starch synthase I (SSI)
“LINKR” Domain Alignments with other similar proteins

| SEQ | Accession | a.a | | a.a. |
|---|---|---|---|---|
| Id.No. | Number | # | (start) Sequence ending | # |
| 336 | MaizeSSI | 332 | ALEW------VFPEWARRHALD---KG-------EAVNFLKGAVVTADRIVTVSKGYSWE | 375 |
| 337 | 7489712 | 332 | ALEW------VFPEWARRHALD---KG-------EAVNFLKGAVVTADRIVTVSKGYSWE | 375 |
| 338 | 12019656 | 339 | ALEW------VFPEWARRHALD---KG-------EAVNFLKGAVVTADRIVTVSKGYSWE | 382 |
| 339 | 1549232 | 333 | ALEW------VFPEWARRHALD---KG-------EAVNFLKGAVVTADRIVTVSQGYSWE | 376 |
| 340 | 2833377 | 333 | ALEW------VFPEWARRRALD---KG-------EAVNFLKGAVVTADRIVTVSQGYSWE | 376 |
| 341 | 5295947 | 333 | ALEW------VFPEWARRHALD---KG-------EAVNFLKGAVVTADRIVTVSQGYSWE | 376 |
| 342 | 9369336 | 339 | ALEW------VFPEWARREALD---KG-------EAVNFLKGAVVTADRIVTVSQGYSWE | 382 |
| 343 | 6103327 | 339 | ALEW------VFPEWARRRALD---KG-------EAVNFLKGAVVTADRIVTVSQGYSWE | 382 |
| 344 | 9369334 | 339 | ALEW------VFPEWARPRALD---KG-------EAVNFLKGAVVTADRIVTVSQGYSWE | 382 |
| 345 | 5880466 | 339 | ALEW------VFPEWARRHALD---KG-------EAVNFLKGAVVTADRIVTVSQGYSWE | 382 |
| 346 | 7188796 | 335 | ALEW------VFPEWARRHALD---KG-------EAVNFLKGAVVTADRIVTVSQGYSWE | 378 |
| 347 | 2833387 | 193 | ALEW------VFPEWARRHALD---KG-------EAVNFLKGAVVTADRIVTVSQGYSWE | 236 |
| 348 | 15237934 | 343 | AVGW------VFPTWARTHALD---TG-------EAVNVLKGAIVTSDRIITVSQGYAWE | 386 |
| 349 | 6690399 | 266 | AVGW------VFPTWARTHALD---TG-------EAVNVLKGAIVTSDRIITVSQGYAWE | 309 |
| 350 | 2829792 | 332 | AVEW------IFPTWAPAHALD---TG-------ETVNVLKGAIAVADRILTVSQGYSWE | 375 |
| 351 | 15232051 | 494 | SFKL------YDPV-----------GG-------EHFNIFAAGLKAADRVLTVSHGYSWE | 529 |
| 352 | 3192881 | 332 | LFKL------YDPV-----------GG-------DHFNIFAAGLKTADRVVTVSHGYAWE | 367 |
| 353 | 2833384 | 454 | LFKM------YDPV-----------GG-------EHFNIFAAGLKTADRIVTVSHGYAWE | 489 |
| 354 | 2129898 | 454 | LFKM------YDPV-----------GG-------EHFNIFAAGLKTADRIVTVSHGYAWE | 489 |
| 355 | 6467503 | 452 | -------------DLFKLHDFI---GG-------DHFNIFAPGLKVADRVVTVSHGYAWE | 488 |
| 356 | 14495348 | 451 | LFKL------YDPF-----------GG-------DHLNIFAAGIKAADRLLTVSHGYAWE | 486 |
| 357 | 7489711 | 400 | HF--------------KLYDNI---GG-------DHSNVFAAGLKTADRVVTVSNGYNWE | 435 |
| 358 | 8708896 | 275 | -----------YRERFRLYDPI---GG-------EHMNVMKAGLECAHRLVAVSKCYAWE | 313 |
| 359 | 15384987 | 339 | HFRL------YDPV-----------GG-------EHSNVFAAGLKMADRAVTVSHGYLWE | 374 |
| 360 | 15028467 | 396 | HFRL------YDPV-----------GG-------EHSNVFAAGLKMADPAVTVSHGYLWE | 431 |
| 361 | 7489710 | 434 | HFRL------YDPV-----------GG-------EHANIFAAGLKMADRVVTVSRGYLWE | 469 |
| 362 | 2833390 | 489 | PFKL------YDPV-----------GG-------EHFNIFAAGLKTADRVVTVSHGYSWE | 524 |
| 363 | 7489695 | 192 | -------------EHFRLYDPV---GG-------EHANYFAAGLKMADQVVVVSPGYLWE | 228 |
| 364 | 8953573 | 499 | -------------EHFRLYDPV---GG-------EHANYFAAGLKMADQVVVVSPGYLWE | 535 |
| 365 | 8953571 | 500 | -------------EHFRLYDFV---GG-------EHANYFAAGLKMADQVVVVSPGYLWE | 536 |
| 366 | 7529653 | 500 | -------------EHFRLYDPV---GG-------EHANYFAAGLKMADQVVVTSPGYLWE | 536 |
| 367 | 16265834 | 512 | HFKLY-------YDPV---------GG-------EHANIFGAGLKMADRVVVTSPGYLWE | 547 |
| 368 | 5825480 | 500 | -------------EHFRLYDPV---GG-------EHANYFAAGLKMADQVVVVSPGYLWE | 536 |
| 369 | 2833388 | 288 | SFDF------I----DGYEKPV---KG-------RKINWMKAGILESDRVLTVSPYYAQE | 327 |
| 370 | 5441242 | 286 | AFD--------FTDGHLKPV-----RG-------RKINWMKAAILESDLVLTVSPYYAKE | 325 |
| 371 | 12003285 | 284 | SFD--------FMDGYEKPV-----KG-------RKINWMKAGIIESDRVLTVSPYYAKE | 323 |
| 372 | 267196 | 287 | SFDF------I----DGYEKPV---KG-------RKINWMKAGILESHRVVTVSPYYAQE | 326 |
| 373 | 228210 | 287 | SFDF------I----DGYEKPV---KG-------RKINWMKAGILESHRVVTVSFYYAQE | 326 |
| 374 | 602594 | 287 | SFDF------I----DGYEKPV---KG-------RKINWMKAGILESHRVVTVSPYYAQE | 326 |
| 375 | 16716335 | 253 | KLAFSDGYAKVYTEATPMEEDE---KPPLTGKTYKKINWLKGGIIAADKLVTVSPNYATE | 309 |
| 376 | 6136121 | 288 | SFDF-------FDGYEKP---V---KG-------RKINWMKAGILESDRVVTVSPYYAQE | 327 |
| 377 | 15223331 | 290 | SFD--------FMDGYEKPV-----KG-------RKINWMKAAILEAHRVLTVSPYYAQE | 329 |
| 378 | 15626365 | 293 | SFDFLDGH--VKPIVGRK------------------INWMKAGIIESHRVLTVSPYYAQE | 332 |
| 379 | 3832512 | 287 | SFDF------I-------DGYDKPVKG-------RKINWNKAGVLESDRVFTVSPYYAKE | 326 |
| 380 | 2833381 | 288 | SFDF------I-------DGYDKPVKG-------RKINWMKAGIREADRVFTVSPNYAYE | 327 |
| 381 | 15637079 | 288 | SFDF------I-------DGYDKFVKG-------RKINWMKAGIREALRVFTVSPNYAKE | 327 |
| 382 | 2833383 | 283 | SFDF------I----DGYNKPC---EG-------RKINWMKAGILESDQVFTVSPHYAKE | 322 |
| 383 | 297424 | 289 | SFDF------I-------DGYDTPVEG-------RKINWMKAGILEADRVLTVSPYYAEE | 328 |
| 384 | 82478 | 289 | SFDF------I-------DGYDTPVEG-------RKINWMKAGILEADRVLTVSPYYAEE | 328 |
| 385 | 2833385 | 288 | SFDF------I----DGYEKPV---EG-------RKINWMKAGILEADRVLTVSPYYAEE | 327 |
| 386 | 2833382 | 289 | SFDF------I-------DGYDTPVEG-------RKINWNKAGILEADRVLTVSPYYAEE | 328 |

TABLE XVI
Maize soluble starch synthase I (SSI)
“GLYTR” Domain Alignments with other similar proteins

| SEQ | Accession | a.a | | a.a. |
|---|---|---|---|---|
| Id.No. | Number | # | (start) Sequence end | # |
| 387 | NaizeSSI | 376 | -VTT-A-EG-GQGLNELLSSRKSVLNGIVNGIDINDWNPATDKCIP-----CHY-SVDD- | 424 |
| 388 | 7489712 | 376 | -VTT-A-EG-GQGLNELLSSRKSVLNGIVNGIDINDWNPATDKCIP-----CHY-SVDD- | 424 |
| 389 | 12019656 | 383 | -VTT-A-EG-GQGLNELLSSRKSVLNGIVNGIDINDWNPATDKCIP-----CHY-SVDD- | 431 |
| 390 | 1549232 | 377 | -VTT-A-EG-GQGLNELLSSRKSVLNGIVNGIDINDWNPSTDKFLP-----YHY-SVDD- | 425 |
| 391 | 2833377 | 377 | -VTT-A-EG-GQGLNELLSSRKSVLNGIVNGIDINDWNPSTDKFLP-----YHY-SVDD- | 425 |
| 392 | 5295947 | 377 | -VTT-A-EG-GQGLNELLSSRKSVLNGIVNGIDINDWNPSTDKFLP-----YHY-SVDD- | 425 |
| 393 | 9369336 | 383 | -VTT-A-EG-GQGLNELLSSRKSVLNGIVNGIDINDWNPTTDKCLP-----HHY-SVDD- | 431 |
| 394 | 6103327 | 383 | -VTT-A-EG-GQGLNELLSSRKSVLNGIVNGIDINDWNPTTDKCLP-----HHY-SVDD- | 431 |
| 395 | 9369334 | 383 | -VTT-A-EG-GQGLNELLSSRKSVLNGIVNGIDINDWNPTTDKCLP-----HHY-SVDD- | 431 |
| 396 | 5880466 | 383 | -VTT-A-EG-GQGLNELLSSRKSVLNGIVNGIDINDWNPTTDKCLP-----HHY-SVDD- | 431 |
| 397 | 7188796 | 379 | -VTT-A-EG-GQGLNELLSSRKSVLNGIVNGIDINDWNPTTDKCLP-----HHY-SVDD- | 427 |
| 398 | 2833387 | 237 | -VTT-A-EG-GQGLNELLSSRKSVLNGIVNGIDINDWNPTTDKCLP-----HHY-SVDD- | 285 |
| 399 | 15237934 | 387 | -ITT-V-EG-GYGLQDLLSSRKSVINGITNGIDINEWNPSTDEHIP-----FHY-SADD- | 435 |
| 400 | 6690399 | 310 | -ITT-V-EG-GYGLQDLLSSRKSVINGITNGINVDEWNPSTDEHIP-----FHY-SADD- | 358 |
| 401 | 2829792 | 376 | -ITT-P-EG-GYGLHELLSSRQSVLNGITNGIDVNDWNPSTDEHIP-----SHY-SIND- | 424 |
| 402 | 15232051 | 530 | -VKT-L-EG-GWGLHNIINENDWKFRGIVNGIDTQEWNPEFDTYLHSDDY-TNY-SLEN- | 582 |
| 403 | 3192881 | 368 | -LKT-S-EG-GWGLNGIRNENEWKLQGIVNGIDIEEWNPQLDVYLKSDGY-ANY-SLDT- | 420 |
| 404 | 2833384 | 490 | -LKT-S-EG-GWGLHNIINESDWKFRGIVNGVDTKDWNPQFDAYLTSDGY-TNY-NLKT- | 542 |
| 405 | 2129898 | 490 | -LKT-S-EG-GWGLHNIINESDWKFRGIVNGVDTKLWNPQFDAYLTSDGY-TNY-NLKT- | 542 |
| 406 | 6467503 | 489 | -LKT-S-EG-GWGLHNIINENHWKLQGIVNGIDAKEWNPQFDIQLTSDGY-TNY-SLET- | 541 |
| 407 | 14495348 | 487 | -LKT-A-EG-GWGLHGIINESDWKFQGIVNGIDTTDWNPRCDIHLKSDGY-TNY-SLET- | 539 |
| 408 | 7489711 | 436 | -LKT-S-EG-GWGLNDIINQNDWKLQGIVNGIDMSEWNPAVD---------VHL-HSDD- | 480 |
| 409 | 8708896 | 314 | -CQT-V-EG-GWGLHEVIIKNNWKLRGIVNGIDYKEWNPICDEFLTTDGY-AHY-DVDT- | 366 |
| 410 | 15384987 | 375 | -IKT-M-DG-GWGLHEIINIIDWKLQGIVNGIDMAEWNPEVDEHLQSDGY-ANY-TFET- | 427 |
| 411 | 15028467 | 432 | -IKT-M-DG-GWGLHEIINENDWKLQGIVNGIDMAEWNPEVDEHLQSDGY-ANY-TFET- | 484 |
| 412 | 7489710 | 470 | -LKT-V-EG-GWGLHDIIRSNDWKINGIVNGIDHQEWNPKVDVHLRSDGY-TNY-SLET- | 522 |
| 413 | 2833390 | 525 | -LKT-S-QG-GWGLHQIINENDWKLQGIVNGIDTKEWNPELDNHLPRSDGYMNY-SLDT- | 578 |
| 414 | 7489695 | 229 | -LKT-V-EG-GWGLHDIIRQNDWKTRGIVNGIDNMEWNPEVDAHLKSDGY-TNF-SLRT- | 281 |
| 415 | 8953573 | 536 | -LKT-V-EG-GWGLHDIIRQNDWKTRGIVNGIDNMEWNPEVDAHLKSDGY-TNF-SLGT- | 588 |
| 416 | 8953571 | 537 | -LKT-V-EG-GWGLHDIIRQNDWKTRGIVNGIDNNEWNPEVDVHLKSDGY-TNF-SLST- | 589 |
| 417 | 7529653 | 537 | -LKT-V-EG-GWGLHDIIRQNDWKTRGIVNGIDNNEWNPEVDVHLKSDGY-TNF-SLRT- | 589 |
| 418 | 16265834 | 548 | -LKT-T-EG-GWGLHDIIRENDWKTRGIVGIDYREWNPEVDVHLQSDGYY-ANY-TVAS- | 600 |
| 419 | 5825480 | 537 | -LKT-V-EG-GWGLHDIIRQNDWTRGIVNGIDNMEWNPEVDVHLQSDGYY-TNF-SLGT- | 589 |
| 420 | 2833388 | 328 | -VIS-GVER-GVELDNFI--RKTGIAGIINGMDVQEWNPVTDKYID-----IHY-DATTV | 376 |
| 421 | 5441242 | 326 | -LVS-G-EDRGVELDNII--RKTGVAGIVNGMDIREWSPKTDKYID-----IHF-DTTS- | 373 |
| 422 | 12003285 | 324 | LVSG-P-DK-GVELDNIL--RKCTVTGIVNGMDTQEWNPATDKYID-----NHY-DITTV | 372 |
| 423 | 267196 | 327 | -LVS-AVDK-GVELDSVL--RKTCITGIVNGMDTQEWNPATDKYTD-----VKY-DITTV | 375 |
| 424 | 228210 | 327 | -LVS-AVDK-GVELDSVL--RKTCITGIVNGMDTQEWNPATDKYTD-----VKY-DITTV | 375 |
| 425 | 602594 | 327 | -LVS-AVDK-GVELDSVL--RKTCITGIVNGMDTQEWNPATDKYTD-----VKY-DITTV | 375 |
| 426 | 16716335 | 310 | -IAADA-AG-GVELDTVI--RAKGIEGIVNGMDIEEWNPKTDKFLS-----VPY-DQNS- | 357 |
| 427 | 6136121 | 328 | LVSG-A-EK-GVELDNVIA--KTSITGIVNGMDTQEWNPATDKHID-----TNY-DITTV | 376 |
| 428 | 15223331 | 330 | LISG-V-DR-GVELHKYL--RMKTVSGIINGMDVQEWNPSTDKYID-----IKY-DITT- | 377 |
| 429 | 15626365 | 333 | LVSG-P-DK-GVELDNIL--RRVGVTGIVNGMDVQEWNPSTDKYIS-----IKY-DASTV | 381 |
| 430 | 3832512 | 327 | -LVS-G-EDRGVELDNII--RSIGITGIVNGMDNREWSPQTDRYID-----VHY-DAST- | 374 |
| 431 | 2833381 | 328 | LVSC-V-SK-GVELDNHI--RDCGITGICNGMDTQEWNPATDKYLA-----VKY-DITTV | 376 |
| 432 | 15637079 | 328 | LVSC-V-SK-GVELDNHI--RDCGITGICNGMDTQEWNPATDKYLA-----VKY-DITTV | 376 |
| 433 | 2833383 | 323 | -LIS-G-EDRGVELDNII--RSTGIIGIVNGMDNREWSPQTDRYID-----VHY-NETT- | 370 |
| 434 | 297424 | 329 | -LIS-G-IARGCELDNIM--RLTGITGIVNGMDVSEWDPSKDKYIT-----AKYDATTA- | 377 |
| 435 | 82478 | 329 | -LIS-G-IARGCELDNIM--RLTGITGIVNGMDVSEWDPSKDKYIT-----AKYDATTA- | 377 |
| 436 | 2833385 | 328 | -LIS-G-IARGCELDNIM--RLTGITGIVNGMDVSEWDPSKDKYIA-----VKY-DVSTA | 376 |
| 437 | 2833382 | 329 | -LIS-G-IARGCELDNIM--RLTGITGIVNGMDVSEWDPSKDKYIT-----AKYDATTA- | 377 |
| 387 | Maize SSI | 425 | L---------S-GKAKCKGALQKELGLPIRPDVPLIGFIGRLDYQKGIDLIQLIIPDLM- | 473 |
| 388 | 7489712 | 425 | L---------S-GKAKCKGALQKELGLPIRPDVPLIGFIGRLDYQKGIDLIQLIIPDLM- | 473 |
| 389 | 12019656 | 432 | L---------S-GKAKCKSALQKELGLPIRPEVPLIGFIGRLDYQKGIDLIQLIIPHLM- | 480 |
| 390 | 1549232 | 426 | L---------S-GKAKCKAELQKELGLPIRPDVPLIGFIGRLDYQKGIDLIQLIIPDLM- | 474 |
| 391 | 2833377 | 426 | L---------S-GKAKCKAELQKELGLPIRPDVPLIGFIGRLDYQKGIDLIKLAIPDLM- | 474 |
| 392 | 5295947 | 426 | L---------S-GKAKCKAELQKELGLPIRPDVPLIGFIGRLDYQKGIDLIKLAIPDLM- | 474 |
| 393 | 9369336 | 432 | L---------S-GKAKCKAELQKELGLPVREDVPLIGFIGRLDYQKGIDLIKMAIPELM- | 480 |
| 394 | 6103327 | 432 | L---------S-GKAKCKAELQKELGLPVREDVPLIGFIGRLDYQKGIDLIKMAIPELM- | 480 |
| 395 | 9369334 | 432 | L---------S-GKAKCKAELQKELGLPVREDVPLIGFIGRLDYQKGIDLIKMAIPELM- | 480 |
| 396 | 5880466 | 432 | L---------S-GKAKCKAELQKELGLPVREDVPLIGFIGRLDYQKGIDLIKMAIPELM- | 480 |
| 397 | 7188796 | 428 | L---------S-GKAKCKAELQRELGLPVREDVPLIGFIGRLDYQKGIDLIKMAIPDLM- | 476 |
| 398 | 2833387 | 286 | L---------S-GKAKCKAELQKELGLPVREDVPLIGFIGRLDYQKGIDLIKMAIPELM- | 334 |
| 399 | 15237934 | 436 | V---------S-EKIKCKMALQKELGLPIRFECPMIGFIGRLDYQKGIDLIQTAGPDLM- | 484 |
| 400 | 6690399 | 359 | V---------S-EKIKCKMALQKELGLPIRPECPMIGFIGRLDYQKGIDLIQTAGPDLM- | 407 |
| 401 | 2829792 | 425 | L---------S-GKVQCKTDLQKELGLPIRPDCPLIGFIGRLDYQKGVDIILSAIFELM- | 473 |
| 402 | 15232051 | 583 | L---------HIGKPQCKAALQKYLGLFVRPDVPLIGFIGRLDHQKGVDLIAEAVPWMM- | 632 |
| 403 | 3192881 | 421 | LQ--------T-GKPQCKAALQKEMNLPVRDDVPLIGFIGRLDHQKGVDLIAEAIPWMM- | 470 |
| 404 | 2833384 | 543 | LQ--------T-GKRQCKAALQRELGLFVREDVPIISFIGRLDHQKGVDLIAEAIPWMM- | 592 |
| 405 | 2129898 | 543 | LQ--------T-GKRQCKAALQRELGLPVREDVPIISFIGRLDHQKGVDLIAEAIPWMM- | 592 |
| 406 | 6467503 | 542 | LD--------T-GKPQCKTALQNELRFAIPPDVPVIGFIGRLDYQKGVDLIAEAIPWMM- | 591 |
| 407 | 14495348 | 540 | VQ--------A-GKQQCKAALQKELGLPVRGDVFVIAFIGRLDHQKGVDLIAEAMPWIA- | 589 |
| 408 | 7489711 | 481 | YTNYTFETLDT-GKRQCKAALQRQLGLQVPDDVPLIGFIGRLDHQKGVDIIADAIHWIA- | 538 |
| 409 | 8708896 | 367 | L---------AEGKAKCKAALQKELGLPVDPDAPMLGFIGRLDYQKGVDLIRDNYDYIM- | 416 |
| 410 | 15384987 | 428 | LD--------T-GKKQCKEALQRQLGLQVRDDVPLIGFIGRLDHQKGVDIIGDAYPWIA- | 477 |
| 411 | 15028467 | 485 | LD--------T-GKKQCKEALQRQLGLQVRDDVPLIGFIGRLDHQKGVDIIGDPYPWIA- | 534 |
| 412 | 7489710 | 523 | LD--------A-GKRQCKAAJJQRELGLEVRDDVPLLGFIGRLDGQKGVDIIGDYPWIA- | 572 |
| 413 | 2833390 | 579 | LQ--------T-GKPQCKAALQKELGLPVRDDVPLIGFIGRLDPQKGVDLIAEAVPWDA- | 628 |
| 414 | 7489695 | 282 | LD--------S-GKRQCKEALQRELGLQVRLVPLLGFIGRLDGQKGVEIIAGDAMPWIV- | 331 |
| 415 | 8953573 | 589 | LD--------S-GKRQCKEALQRELGLQVRGDVPLLGFIGRLDGQKGVEIIADAMPWIV- | 638 |
| 416 | 8953571 | 590 | LD--------S-GKRQCKEALQRELGLQVRADVPLLGFIGRLDGQKGVEIIADAMPWIV- | 639 |
| 417 | 7529653 | 590 | LD--------S-GKRQCKEALQRELGLQVRADVPLLGFIGRLDGQKGVEIIADAMPWIV- | 639 |
| 418 | 16265834 | 601 | LD--------S-SKPRCKAALQRELGLEVPDDVPLIGFIGRLDGQKGVDIIGDAMPWIA- | 650 |
| 419 | 5825480 | 590 | LD--------S-GKRQCKEALQRELGLQVRNVPLLGFIGRLDGQKGVEIIADDAMPWIV- | 639 |
| 420 | 2833388 | 377 | M---------D-AKPLLKEALQAEVGLPVDRNVPLIGFIGRLEEQKGSDIFVAAISQLV- | 425 |
| 421 | 5441242 | 374 | VK--------E-AKFLLKEALQAEVGLPVNRDIFLIGFIGRLEEQKGSDILVEAIPKFI- | 423 |
| 422 | 12003285 | 373 | M---------D-GKPLLKEALQAEVGLPVDRNVPLVGFIGRLEEQKGSDILVPALHKFI- | 421 |
| 423 | 267196 | 376 | M---------D-AKPLLKEALQAAVGLPVDKKIPLIGFIGRLEEQKGSDILVAAIHKFI- | 424 |
| 424 | 228210 | 376 | M---------D-AKPLLKEAWQAAVGLPVDKKIPLIGFIGRLEEQKGSDILVAAIHKFI- | 424 |
| 425 | 602594 | 376 | M---------D-AKPLLKEALQAAVGLPVDKKVPLIGFIGRLEEQKGSDILVAAINKFI- | 424 |
| 426 | 16716335 | 358 | VY--------A-GKAAAKEALQAELGLPVDPTAPLFAFIGRLEEQKGVDIILAALPKIIA | 408 |
| 427 | 6136121 | 377 | M---------D-AKPLLKEALQAAVGLPVDKNIPVIGFIGRLEEQKGSDILVAAISKFV- | 425 |
| 428 | 15223331 | 378 | V---------TDAKPLIKEALQAAVGLPVDRDVPVIGFIGRLEEQKGSDILVEAISKFM- | 427 |
| 429 | 15626365 | 382 | L---------E-GKALLKEELQAEVGLPVDKNVPLIAFIGRLEEQKGSDILVEAIPQFI- | 430 |
| 430 | 3832512 | 375 | V---------TEAKAILKEALQAEVGLPVDRNIPVIGFIGRLEEQKGSDILVESIPKFI- | 424 |
| 431 | 2833381 | 377 | M---------Q-AKPLLKEALQAAVGLPVDRNIPLIGFIGRLEEQKGSDILYAAISKFI- | 425 |
| 432 | 15637079 | 377 | M---------Q-AKPLLKEALQAAVGLPVDRNIPLIGFIGRLEEQKGSDILYAAISKFI- | 425 |
| 433 | 2833383 | 371 | V---------TEAKPLLKGTLQAEIGLFVDSSIPLIGFIGRLEEQKGSDILVEAIAKFA- | 420 |
| 434 | 297424 | 378 | I---------E-AKALNKEALQAEAGLPVDRKIPLIAFIGRLEEQKGSDVMAAAIPELM- | 426 |
| 435 | 82478 | 378 | I---------E-AKALNKEALQAEAGLFVDRKIPLIAFIGRLEEQKGPDVMAAAIPELM- | 426 |
| 436 | 2833385 | 377 | V---------E-AKALNKEALQAEVGLFVDRKIPLVAFIGRLEEQKGPDVMMAAIPLLM- | 425 |
| 437 | 2833382 | 378 | I---------E-AKALNKEALQAEAGLPVDRKIPLIAFIGRLEEQKGPDVMAAAIPELM- | 426 |
| 387 | Maize SSI | 474 | REDVQFVMLGSGDPELEDWMRSTESIFKDKFRGWVGFSVPVSHRITAGCDILLMPSRFEP | 533 |
| 388 | 7489712 | 474 | REDVQFVMLGSGDPELEDWMRSTESIFKDKFRGWVGFSVPVSHRITAGCDILLMPSRFEP | 533 |
| 389 | 12019656 | 481 | RDDVQFVMLGSGDPELEDWMRSTESDFKDKFRGWVGFSVPVSHRITAGCDILLMPSRFEP | 540 |
| 390 | 1549232 | 475 | RDNIQFVMLGSGDPGFEGWMRSTESGYRDKFRGWVGFSVPVSNRITAGCDILLMPSRFEP | 534 |
| 391 | 2833377 | 475 | RDNIQFVMLGSGDPGFEGWMRSTESGYRDKFRGWVGFSVPVSHRITAGCDILLMPSRFEP | 534 |
| 392 | 5295947 | 475 | RDNIQFVMLGSGDPGFEGWMRSTESGYRDKFRGWVGFSVPVSHRITAGCDILLMPSRFEP | 534 |
| 393 | 9369336 | 481 | REDVQFVMLGSGDPIFEGWMRSTESSYKDKFRGWVGFSVPVSHRITAGCDILLMPSRFEP | 540 |
| 394 | 6103327 | 481 | REDVQFVMLGSGDPIFEGWMRSTESSYKDKFRGWVGFSVPVSHRITAGCDILLMPSRFEF | 540 |
| 395 | 9369334 | 481 | REDVQFVMLGSGDPIFEGNMRSTESSYKDKFRGNVGFSVPVSHRITAGCDILLMPSRFEP | 540 |
| 396 | 5880466 | 481 | REDVQFVMLGSGDPIFEGWMRSTESSYKDKFRGWVGFSVPVSHRITAGCDILLMFSRFEP | 540 |
| 397 | 7188796 | 477 | REDVQFVMLGSGDFVFEGWMRSTESSYKDKFRGWVGFSVPVSHRITAGCDILLMPSRFEP | 536 |
| 398 | 2833387 | 335 | REDVQFVMLGSGDPIFEGWMRSTESSYKDKFRGWVGFSVPVSHRITAGCDILLMPSRFEP | 394 |
| 399 | 15237934 | 485 | VDDIQFVMLGSGDPKYESWMRSMEETYRDKFRGWVGFNVPISHRITAGCDILLMPSRFEP | 544 |
| 400 | 6690399 | 408 | VDDIQFVMLGSGDFKYESWMRSMEETYRDKFRGWVGFNVFISHRITAGCDILLMPSRFEP | 467 |
| 401 | 2829792 | 474 | QNDVQVVMLGSGEKQYEDWMRHTENLFKDKFRAWVGFNVPVSHRITAGCDILLMPSRFEP | 533 |
| 402 | 15232051 | 633 | SQDVQLVMLGTGRPDLEEVLRQMEHQYRDKAAGWVGFSVKTAHRITAGADILLMPSRFEP | 692 |
| 403 | 3192881 | 471 | GQDVQLVMLGTGRPDLEQMLKQIEGQYGDKVRGWVGFSVKTAHRITAGADILLMPSRFEP | 530 |
| 404 | 2833384 | 593 | SHDVQLVMLGTGRADLEQMLKEFEAQHCDKIRSWVGFSVKIAHRITAGSDILLMPSRFEP | 652 |
| 405 | 2129898 | 593 | SHDVQLVMLGTGRADLEQMLKEFEAQHCDKIRSWVGFSVKMAHRITAGSDILLMPSRFEP | 652 |
| 406 | 6467503 | 592 | GQDVQLVMLGTGRQDLEEMLRQFENQHRDKVRGWVGFSVKTAHRITAGADILLMPSRFEP | 651 |
| 407 | 14495348 | 590 | GQDVQLIMLGTGRQDLEDTLRRLESQHYDRVRGWVGFSIRLAHRITAGADILLMPSRFEP | 649 |
| 408 | 7489711 | 539 | GQDVQLVMLGTGRADLEDMLRRFESSEHSDKVRAWVGFSVPLAHRITAGDILLMPSRFEP | 598 |
| 409 | 8708896 | 417 | GEKCQLVMLGSGRQDLEDALPLMENRNKNQCRGWVGFSNKMAHRITAAADILLMPSRFEP | 476 |
| 410 | 15384987 | 478 | GQDVQVVMLGTGRPDLEEMLRRFESEHNDKVRGWVGFSVLAHRITAAGADVLLMPSRFEP | 537 |
| 411 | 15028467 | 535 | GQDVQVVMLGTGRPDLEEMLRRFESEHNDKVRGWVGFSVQLAERITAGADVLLMPSRFEP | 594 |
| 412 | 7489710 | 573 | GQDVQLVMLGTGRADLERMLQHLEREHPNKVRGWVGFSVQLAERITAGADVLVMPSRFEP | 632 |
| 413 | 2833390 | 629 | GQDVQLVMLGTGRRDLEQMLRQFECQHNDKIRGWVGFSVKTSHRITAGADVLLMFSRFEP | 688 |
| 414 | 7489695 | 332 | SQDVQLVMLGTGRHDLESMLQHFEREHHDKIRGWVGFSVRLAHRITAGADVLLMPSRFEP | 391 |
| 415 | 8953573 | 639 | SQDVQLVMLGTGRHDLEGMLRLHFEREHHDKRGWVGFSVRLAHRITAGADMLLMPSRFEP | 698 |
| 416 | 8953571 | 640 | SQDVQLVMLGTGRHDLESMLRLFEREHHDKVRGWVGFSVRLAHRITAGADALLMPSRFEP | 699 |
| 417 | 7529653 | 640 | SQDVQLVMLGTGRHDLESMLQHFEREHHDKVRGWVGFSVRLAHRITAGALDALMPSRFEP | 699 |
| 418 | 16265834 | 651 | GQDVQLVLLGSGRRDLEVMLQRFEAQHNSKVRGWVGFSVKLAHRITAGADVLVMPSRFEP | 710 |
| 419 | 5825480 | 640 | SQDVQLVMLGTGRHDLESMLRHFEREHNDKVRGWVGFSVRLAHRITAGADALLMPSRFEP | 699 |
| 420 | 2833388 | 426 | EHNVQIVILGTGKKKFEKQIEHLEVIYPDKARGVAKFNVPLAHMITAGADFMLVPSRFEP | 485 |
| 421 | 5441242 | 424 | DQNVQIIILGTGKKSMEKQIEQLEEIYPEKARGIAKFDGPLAHKIIAGSDFIMIPSRFEP | 483 |
| 422 | 12003285 | 422 | EMDVQVVILGTGKKEFEKQIEQLEELYPGKAVGVAKFNVPLAHKITAGADFMLVPSRFEP | 481 |
| 423 | 267196 | 425 | GLDVQIVVLGTGKKEFEQEIEQLEVLYPNKAKGVAKFNVPLAHMITAGADFMLVPSRFEP | 484 |
| 424 | 228210 | 425 | GLDVQIVVLGTGKKEFEQEIEQLEVLYPGKVKGVAKFNVPLAHMITAGADFMLVPSRFEP | 484 |
| 425 | 602594 | 425 | GLDVQIVVLGTGKKEFEQEIEQLEVLYPNKAKGVAKFNVPLAHMITAGADFMLVPSRFEP | 484 |
| 426 | 16716335 | 409 | TPKVQIAILGTGKAAYEKLVNAIGTKYKGRAKGVVKFSAPLAHMLTAGADFNLVPSRFEP | 468 |
| 427 | 6136121 | 426 | GLDVQIIILGTGKKKFEQQIQELEVLYPDKARGVAKFNVPLAHMITAGADFMLVPSRFEP | 485 |
| 428 | 15223331 | 428 | GLNVQMVILGTGKKKMEAQILELEEKFPGKAVGVAKFNVPLAHMITAGADFIIVPSRFEP | 487 |
| 429 | 15626365 | 431 | KENVQIVALGTGKKEMEKQLQQLEISYPDKARGVAKFNVPLAHMMIAGADFILIPSRFEP | 490 |
| 430 | 3832512 | 425 | DQNVQIIVLGTGKKIMEKQILEQLEVTYPGKAGVAKFNSPLAHKIIAGADFIVIPSRFEP | 484 |
| 431 | 2833381 | 426 | SMDVQILILGTGKKKFEQQIEQLEVMYPDKARGVAKFNVPLAHMITAGADFMLIPSRFEP | 485 |
| 432 | 15637079 | 426 | SMDVQILILGTGKKKFEQQIEQLEVMYPDKARGVAKFNVPLAHMITAGADFMLIPSRFEP | 485 |
| 433 | 2833383 | 421 | DENVQIVVLGTGKKIMEKQIEVLEEKYPGKAIGITKFNSPLAHKIIAGADFIVIPSRFEP | 480 |
| 434 | 297424 | 427 | QEDVQIVLLGTGKKKFEKLLKSMEEKYPGKVRAVVKFNAPLAHLIMAGADVLAVPSRFEP | 486 |
| 435 | 82478 | 427 | QEDVQIVLLGTGKKKFEKILKSMEEKYPGKVRAVVKFNAPLAHLIMAGADVLAVPSRFEF | 486 |
| 436 | 2833385 | 426 | EEDIQIVLLGTGKKKFERALMSAEEKYPDKVPAVVKFNAALAHHIMAGADLLAVTSRFEP | 485 |
| 437 | 2833382 | 427 | QEDVQIVLLGTGKKKFEKLLKSMEEKYPGKVPAVVKFNAPLAHLIMAGIDVLAVFSRFEP | 486 |
| 387 | Maize SSI | 534 | CGLNQLYAMQYGTVPVVHATGGLRDTV-ENFNPF-GENG-EQGTGWAFAFLTTENMFVDI | 590 |
| 388 | 7489712 | 534 | CGLNQLYAMQYGTVPVVHATGGLRDTV-ENFNPF-GENG-EQGTGWAFAPLTTENMFVDI | 590 |
| 389 | 12019656 | 541 | CGLNQLYAMQYGTVPVVHATGGLRDTV-ENFNPF-GENG-EQGTGWAFAPLTTENMFVDI | 597 |
| 390 | 1549232 | 535 | CGLNQLYAMQYGTVPVVHGTGGLRDTV-ENFNPF-AEKG-EQGTGWAFSPLTIEKNAVGI | 591 |
| 391 | 2833377 | 535 | CGLNQLYAMQYGTVPVVUGTGGLRDTV-ENFNPF-AEKG-EQGTGWAFSPLTIEKNAVGI | 591 |
| 392 | 5295947 | 535 | CGLNQLYANQYGTVPVVHGTGGLRDTV-ENFNPF-AEKG-EQGTGWAFSPLTIEK---GI | 588 |
| 393 | 9369336 | 541 | CGLNQLYAMQYGTVPVVHGTGGLRDTV-ETFNPF-GAKG-EEGTGWAFSPLTVEKM | 593 |
| 394 | 6103327 | 541 | CGLNQLYAMQYGTVPVVHGTGGLRDTV-ETFNPF-GAKG-EEGTGWAFSPLTVDKM | 593 |
| 395 | 9369334 | 541 | CGLNQLYAMQYGTVPVVHGTGGLRDTV-ETFNPF-GAKG-EEGTGWAFSPLTVDKM | 593 |
| 396 | 5880466 | 541 | CGLNQLYAMQYGTVPVVHGTGGLRDTV-ETFNPF-GAKG-EEGTGWAFSPLTVDKM | 593 |
| 397 | 7188796 | 537 | CGLNQLYAMQYGTVPVVHGTGGLRDTV-ETFNPF-GAKG-EEGTGWAFSPLTVDKM | 589 |
| 398 | 2833387 | 395 | CGLNQLYAMQYGTVPVVHGTGGLRDTV-ETFNPF-GAKG-EEGTGWAFSPLTVDKM | 447 |
| 399 | 15237934 | 545 | CGLNQLYAMRYGTIPVVHGTGGLPDTV-ENFNPYAEGGA-GTGTGWVFTPLSKDSM | 598 |
| 400 | 6690399 | 468 | CGLNQLYAMRYGTIPVVHGTGGLRDTV-ENFNPYAEGGA-GAGTGWVFTPLSKDSM | 521 |
| 401 | 2829792 | 534 | CGLNQLYAMRYGTIPIVHSTGGLRDTV-KDFNPY-AQEGIGEGTGWTFSPLTSEKL | 587 |
| 402 | 15232051 | 693 | CGLNQLYAMNYGTIPVVHAVGGLRDTV-QQFDPY-SET----GLGWTFDSAEAGKLIHAL | 746 |
| 403 | 3192881 | 531 | CGLNQLYAMSYGTVPVVHAVGGLRDTV-QPFDPF-NES----GYGWTFGRAEANQLIDAL | 584 |
| 404 | 2833384 | 653 | CGLNQLYANSYGTVPVVHGVGGLRDTV-QPFNPF-DES----GVGWTFDRAEANKLMAAL | 706 |
| 405 | 2129898 | 653 | CGLNQLYANSYGTVPVVHGVGGLRDTV-QPFNPF-DES----GVGWTFDRAEANKLMAAL | 706 |
| 406 | 6467503 | 652 | CGLNQLYAPMYGTIPVVRAVGGLRDTV-QPFDPF-NES----GLGWTFDSAESHKLIHAL | 705 |
| 407 | 14495348 | 650 | CGLNQLYAMMYGTVPVVHAVGGLRDTV-EHYNPY-EES----GLGWTFEKAEARNLIDAL | 703 |
| 408 | 7489711 | 599 | CGLNQLYAMAYGTVPVVHAVGGLRDTV-APFDPF-NDT----GLGWTFDRAEANRMIDAL | 652 |
| 409 | 8708896 | 477 | CGLNQLYAMAYGTVPIVHSVGGLRDTV-KQYSPF--EN---VGTGWVF | 518 |
| 410 | 15384987 | 538 | CGLNQLYAMAYGTVFVVHAVGGLRDTV-APFDPF-ADT----GLGWTFDRAEANRMIDAL | 591 |
| 411 | 15028467 | 595 | CGLNQLYAMAYGTVFVVHAVGGLRDTV-APFDPF-ADT----GLGWTFDRAEANRMIDAL | 648 |
| 412 | 7489710 | 633 | CGLNQLYAMAYGTVPVVHAVGGLRDTV-APFDPF----G-DAGLGWTFDRAEANKLIEAL | 686 |
| 413 | 2833390 | 689 | CALNQLYAMAYGTVPVVHAVGGLRDTV-QPFDP | 720 |
| 414 | 7489695 | 392 | CGLNQLYAMAYGTVPVVHAVGGLRDTV-PPFDPF-NHS----GLGWTFDRAEAHKLIEAL | 445 |
| 415 | 8953573 | 699 | CGLNQLYAMAYGTVPVVHAVGGLRDTV-PPFDPF-NHS----GLGWTFDRAEAQKLIEAL | 752 |
| 416 | 8953571 | 700 | CGLNQLYAMAYGTVPVVHAVGGLRDTV-PPFDPF-NHS----GLGWTFDRAEAHKLIEAL | 753 |
| 417 | 7529653 | 700 | CGLNQLYAMAYGTVPVVHAVGGLRDTV-PPFDPF-NHS----GLGWTFDRAEAHKLIEAL | 753 |
| 418 | 16265834 | 711 | CGLNQLYAMAYGTVPVVHAVGGLRDTM-SAFDPF-EDT----GLGWTFDRAEFHKLIEAL | 764 |
| 419 | 5825480 | 700 | CGLNQLYAMAYGTVPVVHAVGGVRDTV-PPFDPF-NNS----GLGWTFDPAEAHKLIEAL | 753 |
| 420 | 2833388 | 486 | CGLIQLHAMRYGTVPIVASTGGLRDTV-K-----------EGYTGFQMGALHVECDKIDS | 533 |
| 421 | 5441242 | 484 | CGLVQLHSMPYGTVPIVSSTGGLVDTVQEGFTGF | 517 |
| 422 | 12003285 | 482 | CGLIQLHANRYGTIPICASTGGLVDTVKEGFTGF | 515 |
| 423 | 267196 | 485 | CGLIQLHAMRYGTVPICASTGGLVDTV-K | 512 |
| 424 | 228210 | 485 | CGLIQLHAMRYGTVPICASTGGLVDTV-K | 512 |
| 425 | 602594 | 485 | CGLIQLHAMRYGTVPICASTGGLVDTV-K | 512 |
| 426 | 16716335 | 469 | CGLIQLHANHYGTVPVVASTGGLVDTV-K | 496 |
| 427 | 6136121 | 486 | CGLIQLHAMRYGTIPICASTGGLVDTVTEGFTGF | 519 |
| 428 | 15223331 | 488 | CGLIQLHANRYGTVPIVASTGGLVDTV-KD | 516 |
| 429 | 15626365 | 491 | CGLIQLQAIVIRYGTVPIVASTGGLVDTVKEGFTGF | 524 |
| 430 | 3832512 | 485 | CGLVQLHANFYGTVPIVSSTGGLVDTV-K-----------EGYTGFHVGAFSVECEAVDP | 532 |
| 431 | 2833381 | 486 | CGLIQLHAMRYGTPCICASTGGLVDTV-K | 513 |
| 432 | 15637079 | 486 | CGLIQLHAMRYGTPCICASTGGLVDTV-K | 513 |
| 433 | 2833383 | 481 | CGLVQLHAMPYGTVPIVSSTGGLVDTV-K-----------EGYTGFHAGPFDVECEDVD | 527 |
| 434 | 297424 | 487 | CGLIQLQGMRYGTPCACASTGGLVDTV | 513 |
| 435 | 82478 | 487 | CGLIQLQGMRYGTACACASTGGLVDTV | 513 |
| 436 | 2833385 | 486 | CGLIQLQGMRYGTPCACASTGGLVDTI | 512 |
| 437 | 2833382 | 487 | CGLIQLQGMRYGTPCACASTGGLVDTV | 513 |

TABLE XVII
Maize soluble starch synthase I (SSI)
“CTEND” Domain Alignments
with other similar proteins

| SEQ | Acces- | | | a. |
|---|---|---|---|---|
| Id. | sion | a.a | | a. |
| No. | Number | # | (start) Sequence end | # |
| 438 | MaizeSSI | 591 | ANCNIYIQGTQVLLGRANEARHVKRLHVGPCR | 622 |
| 439 | 7489712 | 591 | ANCNIYIQGTQVLLGRANEARHVKRLHVGPCR | 622 |
| 440 | 12019656 | 598 | ANCNFDIQGAQIFLGRANEEGHVKRLIWGPCR | 629 |
| 441 | 1549232 | 592 | ADGNFDIQGTQVLLGGSNEARHVKRLYMGPCR | 623 |
| 442 | 2833377 | 592 | ADGNFDIQGTQVLLGGSNEARHVKRLYMGPCR | 623 |
| 443 | 5295947 | 589 | ADGNFDIQGTQVLLGGSNEARHVKRLYMGPCR | 620 |
| 444 | 15232051 | 747 | GNC | 749 |
| 445 | 3192881 | 585 | GNC | 587 |
| 446 | 2833384 | 707 | WNC | 709 |
| 447 | 2129898 | 707 | WNC | 709 |
| 448 | 6467503 | 706 | GHC | 708 |
| 449 | 14495348 | 704 | GHC | 706 |
| 450 | 7489711 | 653 | SHC | 655 |
| 451 | 15384987 | 592 | GHC | 594 |
| 452 | 15028467 | 649 | GHC | 651 |
| 453 | 7489710 | 687 | RHC | 689 |
| 454 | 7489695 | 446 | GHC | 448 |
| 455 | 8953573 | 753 | GHC | 755 |
| 456 | 8953571 | 754 | GHC | 756 |
| 457 | 7529653 | 754 | GHC | 756 |
| 458 | 16265834 | 765 | GHC | 767 |
| 459 | 5825480 | 754 | GHC | 756 |
| 460 | 2833388 | 534 | ADVAAIVKTVARALG | 548 |
| 461 | 3832512 | 533 | AD | 534 |

TABLE XVIII
Identities of the Accession Numbers used in Tables. XIV-XVII.

| Accession | Brief Description of sequences | | |
|---|---|---|---|
| Id. | producing significant alignments | score | E-value |
| gi\|7489712\|pir\|\|T01414 | ADPglucose - starch glucosyltransfera . . . | 1189 | 0.0 |
| gi\|12019656\|gb\|AAD45815.2\| | (AF168786) soluble starch syntha . . . | 1145 | 0.0 |
| gi\|1549232\|dbj\|BAA07396.1\| | (D38221) SSS1 [Oryza sativa] >gi . . . | 998 | 0.0 |
| gi\|2833377\|sp\|Q40739\|UGS2_ORYSA | Soluble glycogen [starch] s . . . | 996 | 0.0 |
| gi\|5295947\|dbj\|BAA81848.1\| | (AB026295) ESTs AU075322 (C11109) . . . | 992 | 0.0 |
| gi\|9369336\|emb\|CAB99210.1\| | (AJ292522) starch synthase I-2 [. . . | 953 | 0.0 |
| gi\|6103327\|gb\|AAF03557.1\| | (AF091802) starch synthase I [Aeg . . . | 953 | 0.0 |
| gi\|9369334\|emb\|CAB99209.1\| | (AJ292521) starch synthase I-1 [. . . | 952 | 0.0 |
| gi\|5880466\|gb\|AAD54661.1\| | (AF091803) starch synthase I [Tri . . . | 951 | 0.0 |
| gi\|7188796\|gb\|AAF37876.1\|AF234163_1 | (AF234163) starch synth . . . | 949 | 0.0 |
| gi\|2833387\|sp\|Q43654\|UGS2_WHEAT | Soluble glycogen [starch] s . . . | 884 | 0.0 |
| gi\|15237934\|ref\|NP 197818.1\| | (NM_122336) soluble starch syn . . . | 744 | 0.0 |
| gi\|6690399\|gb\|AAF24126.1\|AF121673_1 | (AF121673) soluble star . . . | 741 | 0.0 |
| gi\|2829792\|sp\|P93568\|UGS2_SOLTU | Soluble glycogen [starch] s . . . | 724 | 0.0 |
| gi\|15232051\|ref\|NP_186767.1\| | (NM_110984) putative glycogen . . . | 447 | e−124 |
| gi\|3192881\|gb\|AAC19119.1\| | (AF068834) starch synthase [Ipomo . . . | 443 | e−123 |
| gi\|2833384\|sp\|Q43093\|UGS3_PEA | Glycogen [starch] synthase, c . . . | 431 | e−120 |
| gi\|2129898\|pir\|\|S61505 | UDPglucose - starch glucosyltransfera . . . | 431 | e−119 |
| gi\|6467503\|gb\|AAF13168.1\|AF173900_1 | (AF173900) granule boun . . . | 430 | e−119 |
| gi\|14495348\|gb\|AAK64284.1\|AF383878_1 | (AF383878) soluble sta . . . | 426 | e−118 |
| gi\|7489711\|pir\|\|T01209 | ADPglucose - starch glucosyltransfera . . . | 421 | e−117 |
| gi\|8708896\|gb\|AAC17970.2\| | (AF026421) soluble starch synthas . . . | 419 | e−116 |
| gi\|15384987\|emb\|CAC59826.1\| | (AJ308110) soluble starch synth . . . | 419 | e−116 |
| gi\|15028467\|gb\|AAK81729.1\|AF395537_1 | (AF395537) soluble sta . . . | 416 | e−115 |
| gi\|7489710\|pir\|\|T01208 | ADPglucose - starch glucosyltransfera . . . | 414 | e−114 |
| gi\|2833390\|sp\|Q43847\|UGS3_SOLTU | Glycogen [starch] synthase, . . . | 410 | e−113 |
| gi\|7489695\|pir\|\|T06798 | probable starch synthase (EC 2.4.1.— . . . | 405 | e−112 |
| gi\|8953573\|emb\|CAB96627.1\| | (AJ269504) starch synthase IIa-3 . . . | 388 | e−107 |
| gi\|8953571\|emb\|CAB96626.1\| | (AJ269503) starch synthase IIa-2 . . . | 388 | e−107 |
| gi\|7529653\|emb\|CAB86618.1\| | (AJ269502) starch synthase IIa-1 . . . | 388 | e−107 |
| gi\|16265834\|gb\|AAL16661.1\|AF419099_1 | (AF419099) putative so . . . | 387 | e−106 |
| gi\|5825480\|gb\|AAD53263.1\|AF155217_1 | (AF155217) starch synth . . . | 387 | e−106 |
| gi\|2833388\|sp\|Q43784\|UGST_MANES | Granule-bound glycogen [sta . . . | 340 | 3e−92 |
| gi\|5441242\|dbj\|BAA82346.1\| | (AB029546) granule-bound starch . . . | 334 | 2e−90 |
| gi\|12003285\|gb\|AAG43519.1\| | AF210699_1 (AF210699) granule-bou . . . | 325 | 7e−88 |
| gi\|267196\|sp\|Q00775\|UGST_SOLTU | Granule-bound glycogen [star . . . | 325 | 8e−88 |
| gi\|228210\|prf\|\|1718316A | granule-bound starch synthase [Sola . . . | 324 | 2e−87 |
| gi\|602594\|emb\|CAA58220.1\| | (X83220) starch (bacterial glycog . . . | 324 | 2e−87 |
| gi\|16716335\|gb\|AAC17969.3\| | (AF026420) granule-bound starch . . . | 322 | 7e−87 |
| gi\|6136121\|sp\|O82627\|UGST_ANTMA | Granule-bound glycogen [sta . . . | 321 | 2e−86 |
| gi\|15223331\|ref\|NP_174566.1\| | (NM_103023) starch synthase, p . . . | 317 | 3e−85 |
| gi\|15626365\|emb\|CAC69955.1\| | (AJ345045) granule-bound starch . . . | 315 | 1e−84 |
| gi\|3832512\|gb\|AAC70779.1\| | (AF097922) granule-bound glycogen . . . | 313 | 4e−84 |
| gi\|2833381\|sp\|Q42857\|UGST_IPOBA | Granule-bound glycogen [sta . . . | 311 | 9e−84 |
| gi\|15637079\|dbj\|BAB68126.1\| | (AB071604) granule-bound starch . . . | 311 | 1e−83 |
| gi\|2833383\|sp\|Q43092\|UGST_PEA | Granule-bound glycogen [starc . . . | 310 | 4e−83 |
| gi\|297424\|emb\|CAA46294.1\| | (X65183) glycogen (starch) syntha . . . | 303 | 4e−81 |
| gi\|82478\|pir\|\|JQ0703 | UDPglucose - starch glucosyltransferase . . . | 303 | 5e−81 |
| gi\|2833385\|sp\|Q43134\|UGST_SORBI | Granule-bound glycogen [sta . . . | 303 | 5e−81 |
| gi\|2833382\|sp\|Q42968\|UGST_ORYGL | Granule-bound glycogen [sta . . . | 302 | 6e−81 |
| gi\|136758\|sp\|P19395\|UGST_ORYSA | Granule-bound glycogen [star . . . | 302 | 6e−81 |
| gi\|15900991\|ref\|NP_345595.1\| | (NC_003028) glycogen synthase . . . | 302 | 6e−81 |
| gi\|7798551\|gb\|AAC61675.2\| | (AF031162) granule-bound starch s . . . | 301 | 1e−80 |
| gi\|15903076\|ref\|NP_358626.1\| | (NC_003098) Glycogen synthase . . . | 301 | 1e−80 |
| gi\|136757\|sp\|P04713\|UGST_MAIZE | Granule-bound glycogen [star . . . | 300 | 2e−80 |
| gi\|4760582\|dbj\|BAA77351.1\| | (AB019623) starch synthase (GBSS . . . | 300 | 4e−80 |
| gi\|11037536\|gb\|AAG27624.1\|AF286320_1 | (AF286320) granule bou . . . | 300 | 4e−80 |
| gi\|6624287\|dbj\|BAA88512.1\| | (AB029064) starch synthase (GBSS . . . | 296 | 3e−79 |
| gi\|6492245\|gb\|AAF14233.1\|AF109395_1 | (AF109395) granule-boun . . . | 296 | 4e−79 |
| gi\|6624281\|dbj\|BAA88509.1\| | (AB029061) starch synthase (GBSS . . . | 296 | 4e−79 |
| gi\|297422\|emb\|CAA45472.1\| | (X64108) starch granule-bound sta . . . | 295 | 9e−79 |
| gi\|6624283\|dbj\|BAA88510.1\| | (AB029062) starch synthase (GBSS . . . | 294 | 1e−78 |
| gi\|6624285\|dbj\|BAA88511.1\| | (AB029063) starch synthase (GBSS . . . | 294 | 2e−78 |
| gi\|4588609\|gb\|AAD26156.1\|AF113844_1 | (AF113844) granule-boun . . . | 294 | 2e−78 |
| gi\|18652407\|gb\|AAL77109.1\|AF474373_6 | (AF474373) granule-bou . . . | 294 | 2e−78 |
| gi\|136755\|sp\|P09842\|UGST_HORVU | Granule-bound glycogen [star . . . | 293 | 3e−78 |
| gi\|4760584\|dbj\|BAA77352.1\| | (AB019624) starch synthase (GBSS . . . | 293 | 4e−78 |
| gi\|6318540\|gb\|AAF06937.1\|AF110374_1 | (AF110374) granule-boun . . . | 293 | 5e−78 |
| gi\|17736918\|gb\|AAL41028.1\| | (AF250137) mutant granule bound . . . | 292 | 7e−78 |
| gi\|6318538\|gb\|AAF06936.1\|AF110373_1 | (AF110373) granule-boun . . . | 290 | 4e−77 |
| gi\|4760580\|dbj\|BAA77350.1\| | (AB019622) starch synthase (GBSS . . . | 290 | 4e−77 |
| gi\|136765\|sp\|P27736\|UGST_WHEAT | Granule-bound glycogen [star . . . | 288 | 2e−76 |
| gi\|15672681\|ref\|NP_266855.1\| | (NC_002662) glycogen synthase . . . | 287 | 3e−76 |
| gi\|17366711\|sp\|Q9CHM9\|GLGA_LACLA | Glycogen synthase (Starch . . . | 286 | 4e−76 |
| gi\|18139611\|gb\|AAL58572.1\| | (AY069940) granule binding starc . . . | 285 | 1e−75 |
| gi\|16080147\|ref\|NP_390973.1\| | (NC_000964) starch (bacterial . . . | 281 | 1e−74 |
| gi\|4588607\|gb\|AAD26155.1\|AF113843_1 | (AF113843) granule-boun . . . | 278 | 1e−73 |
| gi\|7484400\|pir\| \|T07924 | probable starch synthase (EC 2.4.1.- . . . | 277 | 2e−73 |
| gi\|2811062\|sp\|O08328\|GLGA_BACST | Glycogen synthase (Starch [. . . | 270 | 3e−71 |
| gi\|18309046\|ref\|NP_560980.1\| | (NC_003366) glycogen synthase . . . | 263 | 5e−69 |
| gi\|15895507\|ref\|NP_348856.1\| | (NC_003030) Glycogen synthase, . . . | 256 | 6e−67 |
| gi\|15966599\|ref\|NP_386952.1\| | (NC_003047) PROBABLE GLYCOGEN . . . | 248 | 1e−64 |
| gi\|15643657\|ref\|NP_228703.1\| | (NC_000853) glycogen synthase . . . | 247 | 3e−64 |
| gi\|16766821\|ref\|NP_462436.1\| | (NC_003197) glycogen synthase . . . | 242 | 1e−62 |
| gi\|15613648\|ref\|NP_241951.1\| | (NC_002570) starch (bacterial . . . | 241 | 2e−62 |
| gi\|16762766\|ref\|NP_458383.1\| | (NC_003198) glycogen synthase . . . | 240 | 3e−62 |
| gi\|15641730\|ref\|NP_231362.1\| | (NC_002505) glycogen synthase . . . | 239 | 5e−62 |
| gi\|15803938\|ref\|NP_289974.1\| | (NC_002655) glycogen synthase . . . | 237 | 3e−61 |
| gi\|16124067\|ref\|NP_407380.1\| | (NC_003143) glycogen synthase . . . | 235 | 1e−60 |
| gi\|15890897\|ref\|NP_356569.1\| | (NC_003063) AGR_L_1562p [Agrob . . . | 228 | 1e−58 |
| gi\|16119514\|ref\|NP_396220.1\| | (NC_003064) AGR_pAT_410p [Agro . . . | 226 | 8e−58 |
| gi\|17938870\|ref\|NP_535658.1\| | (NC_003306) glycogen synthase . . . | 225 | 9e−58 |
| gi\|9587341\|gb\|AAF89272.1\|AF285997_1 | (AF285997) granule-boun . . . | 222 | 1e−56 |
| gi\|9587301\|gb\|AAF89252.1\|AF285977_1 | (AF285977) granule-boun . . . | 221 | 1e−56 |
| gi\|9587321\|gb\|AAF89262.1\|AF285987_1 | (AF285987) granule-boun . . . | 221 | 2e−56 |
| gi\|9587293\|gb\|AAF89248.1\|AF285973_1 | (AF285973) granule-boun . . . | 221 | 2e−56 |
| gi\|15606118\|ref\|NP_213495.1\| | (NC_000918) glycogen synthase . . . | 220 | 3e−56 |
| gi\|9587343\|gb\|AAF89273.1\|AF285998_1 | (AF285998) granule-boun . . . | 220 | 3e−56 |
| gi\|15602409\|ref\|NP_245481.1\| | (NC_002663) GlgA [Pasteurella . . . | 219 | 7e−56 |
| gi\|9587317\|gb\|AAF89260.1\|AF285985_1 | (AF285985) granule-boun . . . | 218 | 1e−55 |

Maize Soluble Starch Synthase IIa (SSIIa)

Accession: AF019296
NID: g2811133
Mol. wt. (calc) = 80156 Residues = 732

| | | |
|---|---|---|
| 1 | M S S A A V S S S S S T F F L A L A S A S P G G R R R A R V | SEQ. ID. No. 462 |
| 31 | G S S P F H T G A S L S F A F W A P P S P P R A P R D A A L | |
| 61 | V R A E A E A G G K D A P P E R S G D A A R L P R A R R N A | |
| 91 | V S K R R D P L Q P V G R Y G S A T G N T A R T G A A S C Q | |
| 121 | N A A L A D V E I K S I V A A P P T S I V K F P A P G Y R M | |
| 151 | I L P S G D I A P E T V L P A P K P L H E S P A V D G D S N | |
| 181 | G I A P P T V E P L V Q E A T W D F K K Y I G F D E P D E A | |
| 211 | K D D S R V G A D D A G S F E H Y G D N D S G P L A G E N V | |
| 241 | M N V I V V A A E C S P W C K T G G L G D V V G A L P K A L | |
| 271 | A R R G H R V M V V V P R Y G D Y V E A F D M G I R K Y Y K | |
| 301 | A A G Q D L E V N Y F H A F I D G V D F V F I D A P L F R H | |
| 331 | R Q D D I Y G G S R Q E I M K R M T L F C K V A V E V P W H | |
| 361 | V P C G G V C Y G D G N L V F I A N D W H T A L L P V Y L K | |
| 391 | A Y Y R D H G L M Q Y T R S V L V I H N I A H Q Q R G P V D | |
| 421 | E F P Y M D L P E H Y L Q H F E L Y D P V G G E H A N I F A | |
| 451 | A G L K M A D R V V T V S R G Y L W E L K T V E G G W G L H | |
| 481 | D I I R S N D W K I N G I V N G I D H Q E W N P K V D V H L | |
| 511 | R S D G Y T N Y S L E T L D A G K R Q C K A A L Q R E L G L | |
| 541 | E V R D D V P L L G F I G R L D G Q K G V D I I G D A M P W | |
| 571 | I A G Q D V Q L V M L G T G R A D L E R M L Q H L E R E H P | |
| 601 | N K V R G W V G F S V P M A H R I T A G A D V L V M P S R F | |
| 631 | E P C G L N Q L Y A M A Y G T V P V V H A V G G L R D T V A | |
| 661 | P F D P F G D A G L G W T F D R A E A N K L I E A L R H C L | |
| 691 | D T Y R K Y G E S W K S L Q A R G M S Q D L S W D H A A E L | |
| 721 | Y E D V L V K A K Y Q W | |

TABLE XIX
Maize soluble starch synthase IIa (SSIIa)
Alignments with other similar proteins—Transit Peptide

| SEQ | Accession | a.a | | a.a. |
|---|---|---|---|---|
| Id.No. | Number | # | (start) Sequence ending | # |
| 463 | MAIZE SSIIa | 34 | PFHTGXXXXXXXXXXXXXXXXXXXXXXXVRAEAEAGGKDAPPER--SGDAARLPRARRBAV | 91 |
| 464 | 7489710 | 34 | PFHTGASLSFAFWAPPSPPPRAPRDAALVRAEAEAGGKDAPPER--SGDAARLPRARRNAV | 91 |
| 465 | 8953573 | 101 | DAAEGGAPSPPAP--RQEDARLPSMNGMPV | 128 |
| 466 | 8953571 | 102 | DAAEGGAPAPPAP--RQDAARPPSMNGTPV | 129 |
| 467 | 5825480 | 102 | DAAEGGAPAPPAP--RQDAARPPSMNGTPV | 129 |
| 468 | 7529653 | 102 | DAAEGGGPSPPAA--RQDAARPPSMNGMPV | 129 |
| 469 | 16265834 | 78 | AAVERAGEDDDEEEEFSSGAWQPPRSRRGGV | 108 |
| 470 | 7489711 | 38 | --SGAELRLHWARRGPP | 52 |
| 471 | 7188796 | 21 | PAARATAC | 28 |
| 472 | 2833377 | 16 | APQVR--PGRRLRLQRVRRRCV | 35 |
| 473 | 1549232 | 16 | APQVR--PGRRLRLQRVRRRCV | 35 |
| 474 | 5295947 | 16 | APQVR--PGRRLRLQRVRRRCV | 35 |

TABLE XX
Maize soluble starch synthase ha (SSIIa)
“GLASS” Domain Alignments with other similar proteins

| | | | | | | |
|---|---|---|---|---|---|---|
| SEQ | Accession | a.a | | a.a. | | |
| Id.No. | Number | # | (start) | Sequence | end | # |

| | | | | |
|---|---|---|---|---|
| 475 | MaizeSSIIa | 92 | S---K-RRDPLQPVG-RYGSATGNTAR-------------------TG---A-------- | 116 |
| 476 | 7489710 | 92 | S---K-RRDPLQPVG-RYGSATGNTAR-------------------TG---A-------- | 116 |
| 477 | 8953573 | 129 | N---G-ENKSTGGGGATKDSGLPAPAR-------------------AP---Q-------- | 154 |
| 478 | 8953571 | 130 | N---G-ENKSTGGGGATKDSGLPAPAR-------------------AP---H-------- | 155 |
| 479 | 5825480 | 130 | N---G-ENKSTGGGGATKDSGLPAPAR-------------------AP---H-------- | 155 |
| 480 | 7529653 | 130 | N---G-ENKSTGGGGATKDSGLPTPAR-------------------AP---H-------- | 155 |
| 481 | 16265834 | 109 | GKVLK-RRGTVPPVG-RYGSG-GDAARVRGAAAPAPAPTQDAASSKNG---ALLSGRDDD | 162 |
| 482 | 15028467 | 67 | G-EDGVAKAKTKS-------------------AGSSKA-------- | 84 |
| 483 | 7489711 | 53 | Q---D-GAASVRAAA-APAGGESEEAA-------------------KS---S-------- | 77 |
| 484 | 5880466 | 42 | G-RY---VAELSR-------------------EG---P-------- | 53 |
| 485 | 9369334 | 42 | G-RY---VAELSR-------------------EG---P-------- | 53 |
| 486 | 6103327 | 42 | G-RY---VAELSR-------------------EG---P-------- | 53 |
| 487 | 7188796 | 29 | V---V-RARLRRVAR-GRYVA--ELSR-------------------EG---P-------- | 51 |
| 488 | 2833377 | 36 | A---ELSRDG--------GSAHGPLA---------------------------------- | 50 |
| 489 | 1549232 | 36 | A---E-------------------LSR-------------------DG---G-------- | 43 |
| 490 | 5295947 | 36 | A---E-------------------LSR-------------------DG---G-------- | 43 |
| 475 | MaizeSSIIa | 117 | CQNAALADVEIKSI------VAAP--PTSIVKFPAPGY--RMIL---PSGD--I---A-- | 158 |
| 476 | 7489710 | 117 | -AS--CQNAA ADVEIKSI-VAAP--PTSIVKFPAPGY--RMIL---PSGD--I---A-- | 158 |
| 477 | 8953573 | 155 | -PS--SQNRVPVNGENKAN-VASP--PTSIAEVAAPDP--AATI---SISD--K---A-- | 196 |
| 478 | 8953571 | 156 | -PS--TQNRVPVNGENKAN-VASP--PTSIAEVVAPDS--AATI---SISD--K---A-- | 197 |
| 479 | 5825480 | 156 | -PS--TQNRVPVNGENKAN-VASP--PTSIAEVVAPDS--AATI---SISD--K---A-- | 197 |
| 480 | 7529653 | 156 | -PS--TQNRAPVNGENKAN-VASP--PTSIAEAAASDS--AATI---SISD--K---A-- | 197 |
| 481 | 16265834 | 163 | TPA--SRNGSVVTGADKPA-AATP--PVTITKLPAPDS--PVIL---PSVD--K---PQP | 207 |
| 491 | 15384987 | 65 | ---A-- | 65 |
| 482 | 15028467 | 85 | -VA--VQGSTAKADHVEDS-VSSP-------KYVKPA----VAK---QNGE--VVSRA-- | 122 |
| 483 | 7489711 | 78 | -SS--SQAGAVQGSTAKAVDSASP--PNPLTSAPKQSQ--SAANQNGTSGG--S---S-- | 123 |
| 492 | 15232051 | 181 | ASVISSSP-VTSPQKPSDVATNGKPWS--SVVA---SSVDPPY---K-- | 218 |
| 484 | 5880466 | 54 | -AARPAQQQQL-----------AP--PL------VPGFLAPPPP---APAQ--S---P-- | 83 |
| 485 | 9369334 | 54 | -AARPAQQQQL-----------AP--PL------VPGFLAPPPP---APAQ--S---P-- | 83 |
| 486 | 6103327 | 54 | -AARPAQQQQL-----------AP--PL------VPGFLAPPPP---APAQ--S---P-- | 83 |
| 487 | 7188796 | 52 | -AA--RPAQQL-----------AP--PV------VPGFLAPPPP---APAQ--S---P-- | 79 |
| 488 | 2833377 | 51 | --------------------------PAPLVKQP--------VL---PTF---L---V-- | 65 |
| 489 | 1549232 | 44 | -SA--QRPLA----------------PAPLVKQP--------VL---PTF---L---V-- | 65 |
| 490 | 5295947 | 44 | -SA--QRPLA----------------PAPLVKQP--------VL---PTF---L---V-- | 65 |
| 475 | MaizeSSIIa | 159 | ------------------PETVLPAPK----------PLHE---S----------PAVDG | 177 |
| 476 | 7489710 | 159 | ------------------PETVLPAPK----------PLHE---S----------PAVDG | 177 |
| 477 | 8953573 | 197 | ------------------PESVVPAEKAPPSSGSNFVPSAS---A----------PGSDT | 225 |
| 478 | 8953571 | 198 | ------------------PESVVPAEK----------PPPS---SGSNFVVSASAPRLDI | 226 |
| 479 | 5825480 | 198 | ------------------PESVVPAEK----------PPPS---SGSNFVVSASAPRLDI | 226 |
| 480 | 7329653 | 198 | ------------------PESVVPA--------------EE---T----------PPSSG | 212 |
| 481 | 16265834 | 208 | EFVIPDATAPAPPPPGSNPRSSAPLPK----------PDNS---E----------FAEDK | 244 |
| 491 | 15384987 | 66 | ------------------TKSDAPVFK----------PKVD---PSV--------PASKA | 86 |
| 482 | 15028467 | 123 | ------------------TKSDAPVSK----------PKVD---PSV--------PASKA | 143 |
| 483 | 7489711 | 124 | ------------------ASTAAPVSG----------PKAD---H----------PSAPV | 142 |
| 492 | 15232051 | 219 | ------------------PSSVMTSPE----------KTSDPVTS----------PGKPS | 240 |
| 484 | 5880466 | 84 | ------------------APTQPPLPD----------AGVG---E----------LAPDL | 102 |
| 485 | 9369334 | 84 | ------------------APTQPPLPD----------AGVG---E----------LAPDL | 102 |
| 486 | 6103327 | 84 | ------------------APTQPPLPD----------AGVG---E----------LAPDL | 102 |
| 487 | 7188796 | 80 | ------------------APTQPPLPD----------AGVG---E----------LAPDL | 98 |
| 493 | 7489712 | 49 | PAPE----------PLPP---A----------LLAPP | 62 |
| 488 | 2833377 | 66 | ------------------PTSTPPAPTQSP-------APAP---T----------PPPLP | 87 |
| 489 | 1549232 | 66 | ------------------PTSTPPAPTQSP-------APAP---T----------PPPLP | 87 |
| 490 | 5295947 | 66 | ------------------PTSTPPAPTQSP-------APAP---T----------PPPLP | 87 |
| 475 | Maize SSIIa | 178 | ---DSN-------------------GI--A------P----------------------- | 184 |
| 476 | 7489710 | 178 | ---DSN-------------------GI--A------P----------------------- | 184 |
| 477 | 8953573 | 226 | ---VSDVELELKKGAVIVKEAPNPKAL--S------P----------------------- | 251 |
| 478 | 8953571 | 227 | ---DSDVEPELKKGAVIVEEAPNPKAL--S------P----------------------- | 252 |
| 479 | 5825480 | 227 | ---DSDVEPELKKGAVIVEEAPNPKAL--S------P----------------------- | 252 |
| 480 | 7529653 | 213 | SNFESS-------------------AS--A------PGSDTVSDVEQELKKGAVVVEEAP | 245 |
| 481 | 16265834 | 245 | ---SAK-------------------VVESA------PKPKATR----------------- | 259 |
| 491 | 15384987 | 87 | ---EAD-------------------GN--A------------------------------ | 92 |
| 482 | 15028467 | 144 | ---EAD-------------------GN--A------------------------------ | 149 |
| 483 | 7489711 | 143 | ---TKR-------------------EI--D------A----------------------- | 149 |
| 492 | 15232051 | 241 | ---KSR-------------------AG--AFWSDPLP----------------------- | 253 |
| 484 | 5880466 | 103 | ---LLE-------------------GI--A------E----------------------- | 109 |
| 485 | 9369334 | 103 | ---LLE-------------------GI--A------E----------------------- | 109 |
| 486 | 6103327 | 103 | ---LLE-------------------GI--A------E----------------------- | 109 |
| 487 | 7188796 | 99 | ---LLE-------------------GI--A------E----------------------- | 105 |
| 493 | 7489712 | 63 | ---LVP-------------------GF--L------A----------------------- | 69 |
| 488 | 2833377 | 88 | ---DSGV------------------GE--I------E----------------------- | 95 |
| 489 | 1549232 | 88 | ---DSGV------------------GE--I------E----------------------- | 95 |
| 490 | 5295947 | 88 | ---DSGV------------------GE--I------E----------------------- | 95 |
| 475 | MaizeSSIIa | 185 | ------PTVEPLVQEATWDFK--KYIGFDEP---DEAKDDSRVGADDAGSFEH-YGDN-- | 230 |
| 476 | 7489710 | 185 | ------PTVEPLVQEATWDFK--KYIGFDEP---DEAKDDSRVGADDAGSFEH-YGDN-- | 230 |
| 477 | 8953573 | 252 | ------PAA-PAVQQDLWDFK--KYIGFEEP---VEACDDGPAVALDAGSFEH-HQNH-- | 296 |
| 478 | 8953571 | 253 | ------PAA-PAVQEDLWDFK--KYIGFEEP---VEAKDDGWAVADDAGSFEH-HQNH-- | 297 |
| 479 | 5825480 | 253 | ------PAA-PAVQEDLWDFK--KYIGFEEP---VEAKDDGWAVADDAGSFEH-HQNH-- | 297 |
| 480 | 7529653 | 246 | KPKALSPPAAPAVQEDLWDFK--KYIGFEEP---VEAKDDGRAVADDAGSFEH-HQNH-- | 297 |
| 481 | 16265834 | 260 | ------SSPIPAVEEETWDFK--KYFDLNEPDAAEDGDDDDDWADSDASDSEI-DQDD-- | 308 |
| 491 | 15384987 | 93 | ---------QAVESKAALDKK--EDVGVAEP---LEAKADAGGDAGAVSSADD-SENK-- | 135 |
| 482 | 15028467 | 150 | ---------QAVESKAALDKK--EDVGVAEP---LEAKADAGGDAGAVSSADD-SENK-- | 192 |
| 483 | 7489711 | 150 | ------SAVKP--EPAGDDARPVESIGIAEP---VDAKADAAPATDAAASAPYDREDN-- | 196 |
| 494 | 2833384 | 217 | IRTSSLKFE--NFEGANEP--------SSKEVANEAENFES-GGEK-- | 251 |
| 495 | 2129898 | 217 | IRTSSLKFE--NFEGANEP--------SSKEVANEAENFES-GGEK-- | 251 |
| 496 | 15232051 | 254 | ------SYLTKAPQTSTMKTE--KYVE-KTP---DVASSETNEPGKD---------EE-- | 290 |
| 497 | 3192881 | 107 | DHR---ESSSKEIDVGTEDPVN-----EDL-- | 128 |
| 498 | 2833390 | 284 | DE-- | 285 |
| 499 | 14495348 | 247 | D-- | 247 |
| 484 | 5880466 | 110 | ------DSIDSIIVAASEQDS--EIMDANEQ---PQAK---------------------- | 136 |
| 485 | 9369334 | 110 | ------DSIDSIIVAASEQDS--EIMDANEQ---PQAK---------------------- | 136 |
| 486 | 6103327 | 110 | ------DSIDSIIVAASEQDS--EIMDANEQ---PQAK---------------------- | 136 |
| 487 | 7188796 | 106 | ------DSIDTIVVAAS--------------------EQDSEI------------MDA-- | 125 |
| 493 | 7489712 | 70 | ------PPAEPTGEPAS--------TPPPVP---DAGLGD--LGLEPEGIAEG-SIDNTV | 109 |
| 488 | 2833377 | 96 | ------PDLEGLTEDSI-DKT--IFVASEQE---SEIMDVKEQA---------------- | 127 |
| 489 | 1549232 | 96 | ------PDLEGLTEDSI-DKT--IFVASEQE---SEIMDVKEQA---------------- | 127 |
| 490 | 5295947 | 96 | ------PDLEGLTEDSI-DKT--IFVASEQE---SEIMDVKEQA---------------- | 127 |
| 500 | 6136121 | 54 | RNNAKQSRSLVK-KTDN-- | 69 |
| 501 | 15637079 | 65 | EN-- | 66 |
| 475 | Maize SSIIa | 231 | ------DS--GPLAGEN------V-----MNVIVVAAECSPWCKTGGLGDVVGALPKALA | 271 |
| 476 | 7489710 | 231 | ------DS--GPLAGEN------V-----MNVIVVAAECSPWCKTGGLGDVVGALPKALA | 271 |
| 477 | 8953573 | 297 | ------DS--GPLAGEN------V-----MNVVVVAAECSPWCKTGGLGDVAGALPKALA | 337 |
| 478 | 8953571 | 298 | ------DS--GPLAGEN------V-----MNVVVVAAECSPWCKTGGLGDVAGALPKALA | 338 |
| 479 | 5825480 | 298 | ------DS--GPLAGEN------V-----MNVVVVAAECSPWCKTGGLGDVAGALPKALA | 338 |
| 480 | 7529653 | 298 | ------DS--GPLAGEN------V-----MNVVVVAAECSPWCKTGGLGDVAGALPKALA | 338 |
| 481 | 16265834 | 309 | ------DS--GPLAGEN------V-----MNVIVVAAECSPWCKTGGLGDVAGALPKALA | 349 |
| 502 | 7489695 | 1 | NVVVVAAECSPWCKTGGLGDVAGAIPKALA | 30 |
| 491 | 15384987 | 136 | ------ES--GPLAGPN------V-----MNVIVVASECSPFCKTCGLGDVVGALPKALA | 176 |
| 482 | 15028467 | 193 | ------ES--GPLAGPN------V-----MNVIVVASECSPFCKTGGLGDVVGALPKALA | 233 |
| 483 | 7489711 | 197 | ------EP--GPLAGPN------V-----MNVVVVASECAPFCKTGGLGDVVGALPKALA | 237 |
| 494 | 2833384 | 252 | ------PP---PLAGTN------V-----MNIILVSAECAPWSKTGGLGDVAGSLPKALA | 291 |
| 495 | 2129898 | 252 | ------PP---PLAGTN------V-----MNIILVSAECAPWSKTGGLGDVAGSLPKALA | 291 |
| 503 | 6467503 | 253 | PLAGDN------V-----MNVILVAAECAPWSKTGGLGDVAGSLPKALA | 290 |
| 496 | 15232051 | 291 | ------KP--PPLAGAN------V-----MNVILVAAECAPFSKTGGLGDVAGALPKSLA | 331 |
| 497 | 3192881 | 129 | ------KP--PPLAGTN------V-----MNVILVCAECAPWSKTGGLGDVAGALPKALA | 169 |
| 498 | 2833390 | 286 | ------KP--PPLAGTN------V-----MNIILVASECAPWSKTGGLGDVAGALPKALA | 326 |
| 499 | 14495348 | 248 | ------DP--SASASVD------L-----INIILVAAECAPWSKTGGLGDVAGALPKALA | 288 |
| 504 | 8708896 | 78 | PLAGPN------V-----MNVVMVGAECAPWSKTGGLGDVMAALPKALV | 115 |
| 525 | 2833387 | 1 | EAAPYAKSGGLGDVCGSLPKALA | 23 |
| 492 | 15237934 | 139 | EV------V-----NNLVFVTSEAAPYSKTGGLGDVCGSLPIALA | 172 |
| 484 | 5880466 | 137 | V------T-----RSIVFVTGEAAPYAKSGGLGDVCGSLPIALA | 169 |
| 505 | 6690399 | 62 | EV------V-----NNLVFVTSEAAPYSKTGGLGDVCGSLPIALA | 95 |
| 485 | 9369334 | 137 | ----------------V------T-----RSIVFVTGEAAPYAKSGGLGDVCGSLPIALA | 169 |
| 486 | 6103327 | 137 | ----------------V------T-----RSIVFVTGEAAPYAKSGGLGDVCGSLPIALA | 169 |
| 487 | 7188796 | 126 | ------ND--QPLA-KV------T-----RSIVFVTGEAAPYAKSGGLGDVCGSLPIALA | 165 |
| 506 | 2829792 | 132 | NIIFVTAEAAPYSKTGGLGDVCGSLPMALA | 161 |
| 507 | 12019656 | 140 | SIVFVTGEASPYAKSGGLGDVCGSLPVALA | 169 |
| 493 | 7489712 | 110 | VVASEQDS--EIVVGKEQARAKVT-----QSIVFVTGEASPYAKSGGLGDVCGSLPVALA | 162 |
| 488 | 2833377 | 128 | -------------QAKV------T-----RSVVEVTGEASPYAKSGGLGDVCGSLPIALA | 163 |
| 489 | 1549232 | 128 | -------------QAKV------T-----RSVVFVTGEASPYAKSGGLGDVCGSLPIALA | 163 |
| 490 | 5295947 | 128 | -------------QAKV------T-----RSVVFVTGEASPYAKSGGLGDVCGSLPIALA | 163 |
| 500 | 6136121 | 70 | ------GSPLGKIICGT------G-----MNLVFVLAEVGPWSKTGGLGDVVGGLPPAMA | 112 |
| 508 | 5441242 | 80 | -----MNLIFVGAEVAPWSKTGGLGDVLGGLPSALA | 110 |
| 509 | 2833388 | 82 | -----MNLIFVGAEVGPWSKTGGLGDVLGGLPPALA | 112 |
| 510 | 3832512 | 79 | N------G-----MNLVFVGAEVGPWSKTGGLGDVLGGLPPALA | 111 |
| 511 | 15223331 | 75 | A--GKIVCEK------G-----MSVIFIGAEVGPWSKTGGLGDVLGGLPPALA | 114 |
| 512 | 2833383 | 77 | -----MSLVEVGAEVCPWSKTGGLGDVLGGLPPVLA | 107 |
| 513 | 15637079 | 67 | ------EG--GMAAGTI------VCKQQGMNLVFVGCEVGPWCKTGGLGDVLGGLPPALA | 112 |
| 514 | 267196 | 81 | -----MNLIFVGTEVGPWSKTGGLGDVLGGLPPALA | 111 |
| 515 | 228210 | 81 | -----MNLIFVGTEVGPWSKTGGLGDVLGGLPPALA | 111 |
| 516 | 602594 | 81 | -----MNLIFVGTEVGPWSKTGGLGDVLGGLPPALA | 111 |
| 517 | 283338 | 182 | -----MNLVFVGCEEGPWCKTGGLGDVLGGLPPALA | 112 |
| 518 | 1200328 | 578 | -----MTLIFVSAECGPWSKTGGLGDVVGGLPPALA | 108 |
| 519 | 1562636 | 587 | -----MNLIFVGTEVAPWSKTGGLGDVLGGLPPALS | 117 |
| 520 | 662428 | 173 | GSG------G-----NNLVFVGAEMAPWSKTGGLGDVLGGLPAAMA | 107 |
| 521 | 662428 | 773 | GSG------G-----MNLVFVGAEMAPWSKTGGLGDVLGGLPPAMA | 107 |
| 522 | 476058 | 473 | GEG------G-----MNLVFVGAEMAPWSKTGGLGDVLGGLPPAMA | 107 |
| 523 | 1813961 | 174 | PIVCSA------G-----MTIIFIATECHPWCKTGGLGDVLGGLPPALA | 111 |
| 524 | 17736918 | 3 | GSG------G-----MNLVFVGAEMAPWSKTGGLGDVLGGLPPAMA | 37 |
| 475 | Maize SSIIa | 272 | RRGHRVMVVVPRY-------GD-------YVEAFDMGIRKYYKAAGQDLEVNYFHAFIDG | 317 |
| 476 | 7489710 | 272 | RRGHRVMVVVPRY-------GD-------YVEAFDMGIRKYYKAAGQDLEVNYFHAFIDG | 317 |
| 477 | 8953573 | 338 | KRGHRVMVVVPRY-------GD-------YEEAYDVGVRKYYKAAGQDMEVNYFHAYIDG | 383 |
| 478 | 8953571 | 339 | KRGHRVMVVVPRY-------GD-------YEEAYDVGVRKYYKAAGQDMEVNYFHAYIDG | 384 |
| 479 | 5825480 | 339 | KRGNRVMVVVPRY-------GD-------YEEAYDVGVRKYYKAAGQDMEVNYFHAYIDG | 384 |
| 480 | 7529653 | 339 | KRGHRVMVVVPRY-------GD-------YEEAYDVGVRKYYKAAGQDMEVNYFHAYIDG | 384 |
| 482 | 16265834 | 350 | RRGHRVMVVVPRY-------GD-------YAEAQDVGIRKYYKAAGQDLEVKYFHAFIDG | 395 |
| 525 | 7489695 | 31 | KRGHRVMVVVPRY-------GD-------YEEAYDVGVRKYYKAAGQDMEVNYFHAYIDG | 76 |
| 491 | 15384987 | 177 | RRGHRVMVVIPRY-------GE-------YAEAKDLGVRKRYRVAGQDSEVSYFHAFIDG | 222 |
| 482 | 15028467 | 234 | RRGERVMVVIPRY-------GE-------YAEAKDLGVRKRYRVAGQDSEVSYFHAFIDG | 279 |
| 483 | 7489711 | 238 | RRGHRVMVVIPRY-------GE-------YAEARDLGVRRRYKVAGQDSEVTYFHSYIDG | 283 |
| 494 | 2833384 | 292 | RRGHRVMIVAPRY-------GN-------YAEANDIGVRKRYKVAGQDMEVTYFHTYIDG | 337 |
| 495 | 2129898 | 292 | RRGHRVMIVAPHY-------GN-------YAEANDIGVRKRYKVAGQDMEVTYFHTYIDG | 337 |
| 503 | 6467503 | 291 | RRGNRVMVVAPRY-------GN-------YVEFQDTGVRKRYKVDGQDFEVSYFQAFIDG | 336 |
| 496 | 15232051 | 332 | RRGERVMVVVPRY-------AB-------YAEAKDLGVRKRYKVAGQDMEVMYFHAFIDG | 377 |
| 497 | 3192881 | 170 | RRGHRVMVVVPLY-------GN-------YAEPQHTGVRKMFKIDGQDMEXNYFHAYIDN | 215 |
| 498 | 2833390 | 327 | RRGHRVMVVAPRY-------DN-------YPEPQDSGVRKIYKVDGQDVDVTYFQALLMD | 372 |
| 499 | 14495348 | 289 | RRGHRVMVVVPMY-------KM-------YAEPQQLGEPRRYQVAGQDMEVIYYHAYIDG | 334 |
| 504 | 8708896 | 116 | RRGHRVMVVVPRY-------EN-------YDNAWETGIRKIYSVFNSNQEVGYFHAFVDG | 161 |
| 525 | 2833387 | 24 | ARGHRVMVVMPRYLNGSSD-KN-------YAKALYTAKHIKIPCFGGSEEVTFFHEYRDN | 75 |
| 492 | 15237934 | 173 | GRGHRVMVISPRYLNGTAALKN-------YARAKDLGIRVTVNCFGGSQEVSFYNEYRDG | 225 |
| 484 | 5880466 | 170 | ARGHRVMVVMPRYLMGSSD-KN-------YAKALYTGKHIKIPCFGGSHEVTFFHEYRDN | 221 |
| 505 | 6690399 | 96 | GRGNRVMVISPRYLNGTAADKN-------YARAKDLGIRVTVNCFGGSQEVSFYHEHRDG | 148 |
| 485 | 9369334 | 170 | ARGMRVMVVMPRYLNGSSD-KN-------YAKALYTGKEIKIPCFGGSHEVTFFHEYRDN | 221 |
| 486 | 6103327 | 170 | ARGHRVMVVMPRYLNGSSD-KN-------YAKALYTAKHIKIPCFGGSHEVTFFNEYRDN | 221 |
| 487 | 7188796 | 166 | ARGHRVMVVMPRYLNGTSD-KN-------YAKALYTGKHIKIPCFGGSHEVTFFEEYRDN | 217 |
| 506 | 2829792 | 162 | ARGNRVMVVSPRY-------LNGGPSDEKYANAVDLDVRATVHCFGDAQEVAFYHEYRAG | 214 |
| 507 | 12019656 | 170 | ARGNRVMVVMPRYLNGTSD-KN-------YANAFYTEKMIRIPCFGGEHEVTFFHEYRDN | 221 |
| 493 | 7489712 | 163 | ARGHRVMVVMPRYLNGTSD-KN-------YANAFYTEKHIRIPCFGGEHEVTFFHEYRDS | 214 |
| 488 | 2833377 | 164 | LRGHRVMVVMPRYMNGALN-KN-------FANAFYTEKHIKIPCFGGENEVTFFHEYRDS | 215 |
| 489 | 1549232 | 164 | LRGHRVMVVMPRYMNGALN-KN-------FANAFYTEKXIKIPCFGGEHEVTFFMEYRDS | 215 |
| 490 | 5295947 | 164 | LRGHRVMVVMPRYMNGALN-KN-------FANAFYTEKHIKIFCFGGEHEVTFFHEYRDS | 215 |
| 500 | 6136121 | 113 | GNGHRVMTVSPRY-------DQ-------YKDAWDTSVVVEIKVGDSIETVRFFHCYKRG | 158 |
| 508 | 5441242 | 111 | EHGERVMTVSPRY-------DQ-------YKDAWDTNVTVEVKVADRIETVRFFHCYKQG | 156 |
| 509 | 2833388 | 113 | ARGHRVMTVSPRY-------DQ-------YKDAWDTSVSVEIKIGDRIETVRFFHSYKRG | 158 |
| 510 | 3832512 | 112 | GMGHRVMTVSPRY-------DQ-------YKDAWDTGVSVEIKVGDRFETVRFFWCYKRG | 157 |
| 511 | 15223331 | 115 | ARGHRVMTVCPRY-------DQ-------YKDAWDTCVVVQIKVGDKVENVRFFHCYKRG | 160 |
| 512 | 2833383 | 108 | GNGHRVMTVSPRY-------DQ-------YKDAWDTNVLVEVKVGDKIETVRFFHCYKRG | 153 |
| 513 | 15637079 | 113 | ARGHRVMTVCPRY-------DQ-------YKDAWDTCVVVEIKIGDRIEPVRFFHSYKRG | 158 |
| 514 | 267196 | 112 | ARGHRVMTTSPRY-------DQ-------YKDAWDTSVAVEVKVGDSIEIVRFFHCYKRG | 157 |
| 515 | 228210 | 112 | ARGHRVNTISPRY-------DQ-------YKDAWDTSVAVEVKVGDSIEIVRFFHCYKRG | 157 |
| 516 | 602594 | 112 | ARGHRVMTISPRY-------DQ-------YKDTWDTSVAVEVKVGDSIEIVRFFHCYKRG | 157 |
| 517 | 2833381 | 113 | ARGHRVMTVCPRY-------DQ-------YKDAWETCVVVEPQVGDRIEPVRFFHSYKRG | 158 |
| 518 | 12003285 | 109 | ANRHRVMTVSPRY-------DQ-------YKDAWDTSVVVEIQVGDKVETVGFFHCYKRG | 154 |
| 519 | 15626365 | 118 | ANGHRVMTVTPRY-------DQ-------YKDAWDTNVTIEVKVGDRTEKVRFFHCFKRG | 163 |
| 520 | 6624281 | 108 | ANGHRVMVISPRY-------DQ-------YKDAWDTSVISEIKVVDRYERVRYFHCYKRG | 153 |
| 521 | 6624287 | 108 | ANGHRVMVISPRY-------DQ-------YKDAWDTSVVSEIKVALEYERVRTFHCYKRG | 153 |
| 522 | 4760584 | 108 | ANGNRVMVISPRY-------DQ-------YKDAWDTSVVSEIKVALKYERVRYFHCYKRG | 153 |
| 523 | 18139611 | 112 | AMGHRVMTIVPRY-------DQ-------YKDAWDTNVLVEVNIGDRTETVRFFHCYKRG | 157 |
| 524 | 17736918 | 38 | ANGHRVMVISPRY-------DQ-------YKDAWDTSVVSEIKVVDKYERVRYFHCYKRG | 83 |
| 475 | Maize SSIIa | 318 | VDFVFIDAPLFRH---R-----QDDIY----G---G----SR----QEIMK--------- | 345 |
| 476 | 7489710 | 318 | VDFVFIDAPLFRH---R-----QDDIY----G---G----SR----QEIMK--------- | 345 |
| 477 | 8953573 | 384 | VDFVFIDAPLFRH---R-----QEDIY----G---G----SR----QEIMK--------- | 411 |
| 478 | 8953571 | 385 | VDFVFIDAPIFRH---R-----QEDIY----G---G----SR----QEIMK--------- | 412 |
| 479 | 5825480 | 385 | VDFVFIDAPLFRH---R-----QEDIY----G---G----SR----QEIMK--------- | 412 |
| 480 | 7529653 | 385 | VDFVFIDAPLFRH---R-----QEDIY----G---G----SR----QEIMK--------- | 412 |
| 481 | 16265834 | 396 | VDFVFIDAPLFRH---R-----QDDIY----G---G----NR----QEIMK--------- | 423 |
| 502 | 7489695 | 77 | VDFVFIDAPLFRH---R-----QEDIY----G---G----SR----QEIMK--------- | 104 |
| 491 | 15384987 | 223 | VDFVFLEAPPFRH---R-----HNDIY----G---G----ER----FDVLK--------- | 250 |
| 482 | 15028467 | 280 | VDFVFLEAPPFRH---R-----HNDIY----G---G----ER----FDVLK--------- | 307 |
| 483 | 7489711 | 284 | VDFVFVEAPPFRH---R-----HNNIY----G---G----ER----LDILR--------- | 311 |
| 494 | 2833384 | 338 | VDIVFIDSPIFPN---L-----ESNIY----G---G----NR----LDILR--------- | 365 |
| 495 | 2129898 | 338 | VDIVFIDSPIFPN---L-----ESNIY----G---G----NR----LDILR--------- | 365 |
| 503 | 6467503 | 337 | VDFVFIDSPMFRH---I-----GNDIY----G---G----NR----MDILK--------- | 364 |
| 496 | 15232051 | 378 | VDFVFIDSPEFRH---L-----SNNIY----G---G----NR----LDILK--------- | 405 |
| 497 | 3192881 | 216 | VDFVFIDSPIFQH---R-----GNNIY----G---G----NR----VDILK--------- | 243 |
| 498 | 2833390 | 373 | CDFVFIHSHMFRH---I-----GNNIY----G---G----NR----VDILK--------- | 400 |
| 499 | 14495348 | 335 | VDFVFIDNPIFEH---V-----ENDIY----G---G----DR----TDILK--------- | 362 |
| 504 | 8708896 | 162 | VDYVFVDHPTFHG---R-----GKNIY----G---G----ER----QEILF--------- | 189 |
| 488 | 2833387 | 76 | VDWVFVDHPSY-H---R-----PGSLY----GDNFG----AF----GDNQF--------- | 105 |
| 492 | 15237934 | 226 | VDWVFVDHKSY-H---R-----PGNPY----G---D----SK----GAFGDNQF------ | 255 |
| 484 | 5880466 | 222 | VDWVFVDHPSY-H---R-----PGSLY----GDNFG----AF----GDNQF--------- | 251 |
| 505 | 6690399 | 149 | VDWVFVDHKSY-H---R-----PGNPY----G---D----SK----GAFGDNQF------ | 178 |
| 485 | 9369334 | 222 | VDWVFVDHPSY-H---R-----PGSLY----GDNFG----AF----GDNQF--------- | 251 |
| 486 | 6103327 | 222 | VDWVFVDHPSY-H---R-----PGSLY----GDNFG----AF----GDNQF--------- | 251 |
| 487 | 7188796 | 218 | VDWVFVDHPSY-H---R-----PGSLY----GDNFG----AF----GDNQF--------- | 247 |
| 506 | 2829792 | 215 | VDWVFVDNSSYCRPGTP-----YGDIY----GAF-G----DN----Q---F--------- | 244 |
| 507 | 12019656 | 222 | VDWVFVDHPSY-H---R-----PGNLY----GDKFG----AF----GDNQF--------- | 251 |
| 493 | 7489712 | 215 | VDWVFVDNPSY-H---R----RPGNLY----GDKFG----AF----GDNQF--------- | 244 |
| 488 | 2833377 | 216 | VDWVFVDHPSY-H---R-----PGNLY----GDNFG----AF----GDNQF--------- | 245 |
| 489 | 1549232 | 216 | VDWVFVDHPSY-H---R-----PGNLY----GDNFG----AF----GDNQF--------- | 245 |
| 490 | 5295947 | 216 | VDWVFVDHPSY-N---R-----PGNLY----GDNFG----AF----GDNQF--------- | 245 |
| 500 | 6136121 | 159 | VDRVFVDHPIFLE---KVWGKTKSKIY----G---P----NAGTDYQDNQL--------- | 195 |
| 508 | 5441242 | 157 | VDRVFVDHPCFLE---KVWGKTGSKLY----G---P----SAGVDYEDNQL--------- | 193 |
| 509 | 2833388 | 159 | VDRVFVDHPMFLE---KVWGKTGSKIY----G---PRAGLDY----QDNQL--------- | 195 |
| 510 | 3832512 | 158 | VDRVFVDHPLFLE---KVWGKTESKLY----G---P----KTGVDYKNQL---------- | 194 |
| 511 | 15223331 | 161 | VDRVFVDHPIFLA---KVGKTGSKIY----G---PITGVDY----NDNQL---------- | 197 |
| 509 | 2833383 | 154 | VDRVFVDHPLFLE---RVWGKTGSKLY----G---P----KTGIDYRDNQL--------- | 190 |
| 513 | 15637079 | 159 | VDRVFVDHPMFLE---KVWGKTGSMLY----G---P----KA----GKDYKDNQL----- | 195 |
| 514 | 267196 | 158 | VDRVFVDHPMFLE---K--------VW----G---K----TG----SKIYGPKAGLDYLD | 191 |
| 515 | 228210 | 158 | VDRVFVDHPMFLE---K--------VW----G---K----TG----SKIYGPKAGLDYLD | 191 |
| 516 | 602594 | 158 | VDRVFVDHPMFLE---K--------VW----G---K----TG----SKIYGPKAGLDYLD | 191 |
| 517 | 2833381 | 159 | VDRVFVDHPMFLE---KVWGKTGSMLY----G---P----KA----GKDYKDNQL----- | 195 |
| 518 | 12003285 | 155 | VDRVFVDHPLFLE---KVWGKTKSKVY----G---P----SAGVDYEDNQL--------- | 191 |
| 519 | 15626365 | 164 | VDRVFVDHPIFLE---KVWGKTGTKLY----G---P----AAGDDYQDNQL--------- | 200 |
| 520 | 6624281 | 154 | VDRVFVDHPCFLE---KVRGKTKEKIYGPDAG---T----DY----EDNQQ--------- | 190 |
| 521 | 6624287 | 154 | VDRVFVDHPCELE---KVRGKTKEKIY----GPDAG---TD----YEDNQL--------- | 190 |
| 522 | 4760584 | 154 | VDRVFVDHPCFLE---KVRGKTKEKIYGPDAG---T----DY----EDNQQ--------- | 190 |
| 523 | 18139611 | 158 | VDRVFVDHPMFLE---KVWGKTGPKLY----G---F----TT----GDDYRDNQL----- | 194 |
| 524 | 17736918 | 84 | VDRVFVDHPCFLE---KVRGKTKEKIYGPDAG---T----DY----EDNQQ--------- | 120 |
| 475 | (Maize SSIIa) | 346 | ---RMILFCKVAVEVPW-----HVPCGGV-----CYGDG----NLVFIANDWHTALLPVY | 388 |
| 476 | 7489710 | 346 | ---RMILFCKVAVEVPW-----HVPCGGV-----CYGDG----NLVFIANDWHTALLPVY | 388 |
| 477 | 8953573 | 412 | ---RMILFCKAAVEVPW-----HVFCGGV-----PYGDG----NLVFIANDWHTALLPVY | 454 |
| 478 | 8953571 | 413 | ---RMILFCKAAVEVFW-----HVPCGGV-----PYGDG----NLVFIANDWHTALLPVY | 455 |
| 479 | 5825480 | 413 | ---RMILFCKAAVEVPW-----HVPCGGV-----PYGDG----NLVFIANDWHTALLPVY | 455 |
| 480 | 7529653 | 413 | ---RMILFCKAAVEVPW-----HVPCGGV-----PYGDG----NLVFIANDWHTALLPVY | 455 |
| 481 | 16265834 | 424 | ---RMILFCKAAVEVPW-----HVPCGGV-----PYGDG----NLVFLANDWHTALLPVY | 466 |
| 502 | 7489695 | 105 | ---RMILFCKAAVEVPW-----HVPCGGV-----PYGDG----NLVFIANDWHTALLPVY | 147 |
| 491 | 15384987 | 251 | ---RMILFCKAAVEVPW-----FAPCGGS-----IYGDG----NLVFIANDWHTALLPVY | 293 |
| 482 | 15028467 | 308 | ---RMILFCKAAVEVPW-----FAPCGGS-----IYGDG----NLVFIANDWHTALLPVC | 350 |
| 483 | 7489711 | 312 | ---RMILFCKAAVEVPW-----YAPCGGT-----VYGDG----NLVFIANDWHTALLPVY | 354 |
| 494 | 2833384 | 366 | ---RMVLFCKAAVEVPW-----HVPCGGI-----CYGDG----NLVFIANDWHTALLPVY | 408 |
| 495 | 2129898 | 366 | ---RMVLFCKAAVEVPW-----HVPCGGI-----CYGDG----NLVFIANDWHTALLPVY | 408 |
| 503 | 6467503 | 365 | ---RMVLFCKAAVEVPW-----HVPCGGV-----CYGDG----NLAFIANDWHTALLPVY | 407 |
| 492 | 15232051 | 406 | ---RMVLFCKAAVEVPW-----YVPCGCV-----CYGDG----NLAFIANDWHTALLPVY | 448 |
| 497 | 3192881 | 244 | ---RMDLFCKAAIVVPW-----HVPCGGI-----CYGDG----NLVFIANDWNTALLPVY | 286 |
| 498 | 2833390 | 401 | ---RMVLFCKAAIEVPW-----HVPCGGV-----CYGDG----NLVFIANDWHTALLPAY | 443 |
| 499 | 14495348 | 363 | ---RMVLLCKAAIEVPW-----YVPCGGY-----CYGDG----NLVFLANDWHTALLPVY | 405 |
| 504 | 8708896 | 190 | ---RCALLCKAALEAVW-----HVPCGGI-----TYGDD----NLCFIANDWHTALLPVY | 232 |
| 488 | 2833387 | 106 | ---RYTLLCYAACEAFL-----ILELGGY-----IYGQ-----NCMFVVNDWHASLVPVL | 147 |
| 492 | 15237934 | 256 | ---RFTLLCHAACEAPL-----VLFLGGF-----TYGEK----SL-FLVNDWHAGLVPIL | 297 |
| 484 | 5880466 | 252 | ---RYTLLCYAACEAPL-----ILELGGY-----IYGQ-----NCMFVVNDWHASLVPVL | 293 |
| 505 | 6690399 | 179 | ---RFTLLCHAACEAPL-----VLPLGGF-----TYGEK----SL-FLVNDWHAGLVPIL | 220 |
| 485 | 9369334 | 252 | ---RYTLLCYAACEAPL-----ILELGGY-----IYGQ-----NCMFVVNDWHASLVPVL | 293 |
| 486 | 6103327 | 252 | ---RYTLLCYAACEAPL-----ILELGGY-----IYGQ-----NCMFVVNDWHASLVPVL | 293 |
| 484 | 7188796 | 248 | ---RYTLLCYAACEAPL-----ILELGGY-----IYGQ-----SCMFVVNDWHASLVPVL | 289 |
| 506 | 2829792 | 245 | ---RFTLLSHAACEAPL-----VLPLGGF-----TYGEK----CL-FLANDWHAPLVPLL | 286 |
| 507 | 12019656 | 252 | ---RYTLLCYAACEAPL-----VLELGGY-----IYGQ-----NCMFVVNDWHASLVPVL | 293 |
| 493 | 7489712 | 245 | ---RYTLLCYAACEAPL-----ILELGGY-----IYGQ-----NCMFVVNDWHASLVPVL | 286 |
| 488 | 2833377 | 246 | ---RYTLLCYAACEAPL-----ILELGGY-----IYGQ-----KCMFVVNDWHASLVPVL | 287 |
| 489 | 1549232 | 246 | ---RYTLLCYAACEAPL-----ILELGGY-----IYGQ-----KCMFVVNDWHASLVPVL | 287 |
| 490 | 5295947 | 246 | ---RYTLLCYAACEAPL-----ILEEGGY-----IYGQ-----KCMFVVNDWHASLVPVL | 287 |
| 500 | 6136121 | 196 | ---RFSLLCQAALEAPR-----VLNLTSS-----KYFSGPYGEDVVFVANDWHTALLPCY | 242 |
| 508 | 5441242 | 194 | ---RYSLLCQAALEAPR-----VLNLNSN-----KYFSGPYGEDVIFVANDWHTALLPCY | 240 |
| 509 | 2833388 | 196 | ---RFSLLCLAALEAPR-----VLNLNSSKNFSGPYGE-----EVAFIANDWHTALLPCY | 242 |
| 510 | 3832512 | 195 | ---RFSLLCQAALEAPR-----VLNLNSN-----KHFSGPYGEDVVFVANDWHTALLPCY | 241 |
| 511 | 15223331 | 198 | ---RFSLLCQAALEAPQ-----VLNLNSS-----KYFSGPYGEDVVFVANDWHTALLPCY | 244 |
| 512 | 2833383 | 191 | ---RFSLLCQAALEAPR-----VLNLNSS-----KYFSGPYGEDVIFVANDWHSALIPCY | 237 |
| 513 | 15637079 | 196 | ---RFSLLCQAALEAPR-----VLNLNSS-----NYFSGPYGEDVVFVANDWHTALLPCY | 242 |
| 514 | 267196 | 192 | NELRFSLLCQAALEAPK-----VLNLNSS-----NYFSGPYGEDVLFIANDWHTALIPCY | 241 |
| 515 | 228210 | 192 | NELRFSLLCQAALEAPK-----VLNLNSS-----NYFSGPYGEDVLFIANDWHTALIPCY | 241 |
| 516 | 602594 | 192 | NELRFSLLCQAALEAPK-----VLNLNSS-----NYFSGPYGEDVLFIANDWHTALIPCY | 241 |
| 517 | 2833381 | 196 | ---RFSLLCQAALEAPR-----VLNLNSS-----KYFSGPYGEDVVFVANDWHTAWLPCY | 242 |
| 518 | 12003285 | 192 | ---RFSLLSLAALEAPP-----VLNLTSN-----KYFSGPYGEDVVFVANDWETAVLPCY | 238 |
| 519 | 15626365 | 201 | ---RFSIFCQAALEAAR-----VLNLKSN-----KYFSGPYGEDVIFVANDWHTALISCY | 247 |
| 520 | 6624281 | 191 | ---RFSLLCQAALEVPRILDLNNNPHFSG-----PYGE-----DVVFVCNDWHTGLLACY | 237 |
| 521 | 6624287 | 191 | ---RFSLLCQAALEAPR-----ILDLNNN-----PYFSGPYGEDVVFVCNDWHTGLLACY | 237 |
| 522 | 4760584 | 191 | ---RFSLLCQAALEVPR-----ILNLDNN-----PYFSGPYGEDVVFVCNDWHTGLLACY | 237 |
| 523 | 18139611 | 195 | ---RFCLLCLAALEAPR-----VLNLNNS-----EYFSGPYGENVVFVANDWHTGVLPCY | 241 |
| 524 | 17736918 | 121 | ---RFSLLCQAALEVPR-----ILNLDNN-----PYFSGPYGEDVVFVCNDWHTGLLACY | 167 |
| 475 | Maize SSIIa | 389 | LKAYYRDHGLMQYTRSVLVIHNIAHQGRGPVDEFPYMDLPEHYLQHFELYD----PV--- | 441 |
| 476 | 7489710 | 389 | LKAYYRDHGLMQYTRSVLVIHNIAHQGRGPVDEFPYMDLPEHYLQHFELYD----PV--- | 441 |
| 477 | 8953573 | 455 | LKAYYRDHGLMQYTRSVLVIHNIAHQGRGPVDEFPYMDLPEHYLQHFELYD----PV--- | 507 |
| 478 | 8953571 | 456 | LKAYYRDHGLMQYTRSVLVIHNIAHQGRGPVDEFPYMDLPEHYLQHFELYD----PV--- | 508 |
| 479 | 5825480 | 456 | LKAYYRDHGLMQYTRSVLVIHNIAHQGRGPVDEFPYMDLPEHYLQHFELYD----PV--- | 508 |
| 480 | 7529653 | 456 | LKAYYRDHGLMQYTRSVLVIHNIAHQGRGPVDEFPYMDLPEHYLQHFELYD----PV--- | 508 |
| 481 | 16265834 | 467 | LKAYYRDHGLMQYTRSVLVIHNIAHQGRGPVDEFPYMDLPEHYLQHFELYD----PV--- | 519 |
| 502 | 7489695 | 148 | LKAYYRDHGLMQYTRSVLVIHNIAHQGRGPVDEFPYMDLPEHYLQHFELYD----PV--- | 200 |
| 491 | 15384987 | 294 | LKAYYRDHGLMQYTRSVLVIHNIAHQGRGPVDEFPYMDLPEHYLQHFELYD----PV--- | 346 |
| 482 | 15028467 | 351 | LKAYYRDHGLMQYTRSVLVIHNIAHQGRGPVDEFPYMDLPEHYLQHFELYD----PV--- | 403 |
| 483 | 7489711 | 355 | LKAYYRDHGLMQYTRSVLVIHNIAHQGRGPVDEFPYMDLPEHYLQHFELYD----PV--- | 407 |
| 494 | 2833384 | 409 | LKAYYRDHGLMNYTRSVLVIHNIAHQGRGPVEDFNTVDLSGNYLDLFKMYD----PV--- | 461 |
| 495 | 2129898 | 409 | LKAYYRDHGLMNYTRSVLVIHNIAHQGRGPVEDFNTVDLSGNYLDLFKMYD----PV--- | 461 |
| 503 | 6467503 | 408 | LKAYYRDNGLMQYTRSVLVIHNIAHQGRGPSGDFSYVGLPEHYIDLFKLHD----PI--- | 460 |
| 492 | 15232051 | 449 | LKAYYRDHGIMKYTRSVLVIHNIAHQGRGPVDDFSYVDLPSHYLDSFKLYD----PV--- | 501 |
| 497 | 3192881 | 287 | LKAYFRDNGVMKFTRSVLVIHNIAHQGRGPMDDFSIVDLPAQUADLFKLYD----PV--- | 339 |
| 498 | 2833390 | 444 | LKAYYRDNGIMNYTRSVLVIHNIAHQGRGPLEDFSYVDLPPHYMDPFKLYD----PV--- | 496 |
| 499 | 14495348 | 406 | LKAYYHDNGFMIYARSVLVIHNIAHQGRGPLDDFSYLDLPVDYMDLFKLYD----PF--- | 458 |
| 504 | 8708896 | 233 | LQAHYRDYGEMTYARCAFVIHNMAHQGRGPFVESEHLELNEEYRERFRLYD----PI--- | 285 |
| 488 | 2833387 | 148 | LAAKYRPYGVYRDSRSTLVIHNLAHQGVEPASTYPDLGLPPEWYGALEWVF----PEWAR | 203 |
| 492 | 15237934 | 298 | LAAKYRPYGVYKDARSILIIHNLAHQGVEPAATYTNLGLPS------EWYG----AV--- | 344 |
| 484 | 5880466 | 294 | LAAKYRPYGVYRDSRSTLVIHNLAHQGVEPASTYPDLGLPPEWYGALEWVF----PEWAR | 349 |
| 505 | 6690399 | 221 | LAAKYRPYGVYKDARSILIIHNLAHQGVEPAATYTNLGLPS------EWYG----AV--- | 267 |
| 485 | 9369334 | 294 | LAAKYRPYGVYRDSRSTLVIHNLAHQGVEPASTYPDLGLPPEWYGALEWVF----PEWAR | 349 |
| 486 | 6103327 | 294 | LAAKYRPYGVYRDSRSTLVIHNLAHQGVEPASTYPDLGLPPEWYGALEWVF----PEWAR | 349 |
| 487 | 7188796 | 290 | LAAKYRPYGVYRDSRSTLVIHNLAHQGVEPASTYPDLGLPPEWYGALEWVF----PEWAR | 345 |
| 506 | 2829792 | 287 | LAAKYRPYGVYKDARSIVAIHNIAHQGVEPAVTYNNLGLPPQWYGAVEWIF----PTWAR | 342 |
| 526 | 7484400 | 4 | RGRGPFVESEHLELNEEYRERFRLYD----PI--- | 31 |
| 507 | 12019656 | 294 | LAAKYRPYGVYKDSRSILVIHNLAHQGVEPASTYPDLGLPPEWYGALEWVF----PEWAR | 349 |
| 493 | 7489712 | 287 | LAAKYRPYGVYKDSRSILVIHNLAHQGVEPASTYPDLGLPPEWYGALEWVF----PEWAR | 342 |
| 488 | 2833377 | 288 | LAAKYRPYGVYKDSRSILVIHNLAHQGVEPASTYPDLGLPPEWYGALEWVF----PEWAR | 343 |
| 489 | 1549232 | 288 | LAAKYRPYGVYKDSRSILVIHNLAHQGVEPASTYPDLGLPPEWYGALEWVF----PEWAR | 343 |
| 490 | 5295947 | 288 | LAAKYRPYGVYKDSRSILVIHNLAHQGVEPASTYPDLGLPPEWYGALEWVF----PEWAR | 343 |
| 500 | 6136121 | 243 | LKSMYQSKGMYLHAKVAFCIHNIAYQGRFGSSDFCLLNLPDQFKSSFDFFDGYEKPV--- | 299 |
| 508 | 5441242 | 241 | LKSMYQTRGVYRNTKVAFCIHNISYQGRHPFEDFPLLNLPNEYRSAFDFTDGHLKPV--- | 297 |
| 509 | 2833388 | 243 | LKAIYQPMGIYKHAKVAFCIHNIAYQGRFAFSDFFRLNLPDKFKSSFDFIDGYEKPV--- | 299 |
| 510 | 3832512 | 242 | LKSLYKSKGIYKSAKVAFCIHNIAYQGPYAFSDLSLLNLPNEFRSSFDFIDGYDKPV--- | 298 |
| 511 | 15223331 | 245 | LKSMYQSRGVYMNAKVVFCIHNIAYQGRFAFDDYSLLNLPISFKSSFDFMDGYEKPV--- | 301 |
| 512 | 2833383 | 238 | LKSMYKSRGLYKNAKVAFCIHNIAYQGRNAFSDFSLLNLPDEFRSSFDFIDGYNKPC--- | 294 |
| 513 | 15637079 | 243 | LKTMYQSRGIYNNAKVAFCIWNIAYQGRFAFSDFSLLNLPDEYKGSFDFIDGYDKPV--- | 299 |
| 514 | 267196 | 242 | LKSMYQSRGIYLNAKVAFCINNIAYQGRFSFSDFPLLNLPDEFRGSFDFIDGYEKPV--- | 298 |
| 515 | 228210 | 242 | LKSMYQSRGIYLNAKVAFCIHNIAYQGRFSFSDFPLLNLPDEFRGSFDFIDGYEKPV--- | 298 |
| 516 | 602594 | 242 | LKSMYQSRGIYLNAKVAFCIHNIAYQGRFSFSDFPLLNLPDEFRGSFDFIDGYEKPV--- | 298 |
| 517 | 2833381 | 243 | LKTMYQSRGIYMNAKVAFCIHNIAYQGRFAFSDFSLLNLPDEYKGSFDFIDGYDKPV--- | 299 |
| 518 | 12003285 | 239 | LKTIYQPKGIYTNAKVVLCIHNIAYQGRFAFSDFYKLNLPDQLKSSFDFMDGYEKPV--- | 295 |
| 519 | 15626365 | 248 | MKSMYQSIGIFRNAKVVFCIHNIAYQGRFAFTDYSLLNLPDQFKSSFDFLDQWIKPI--- | 304 |
| 520 | 6624281 | 238 | LKSNYQSNGIYRTAKVAFCIHNISYQGRFSFDDFAQLNLPDRFKSSFDFIDGYDKPV--- | 294 |
| 521 | 6624287 | 238 | LKSNYQSNGIYMTAKVAFCIHNISYQGRFSFDDFAQLNLPDRFKSSFDFIDGYDKPV--- | 294 |
| 522 | 4760584 | 238 | LKSNYQSNGIYRAAKVAFCIHNISYQGRFSFDDFAQLNLPDRFKSSFDFIDGYDKPV--- | 294 |
| 523 | 18139611 | 242 | LKSIYQAKGMYThAKVAFCIHNIAYQGRFAREDFELLNLPDSELPSFDFIDGHFKPV--- | 298 |
| 524 | 17736918 | 168 | LKSNYQSNGIYRAAKVAFCIHNISYQGRFSFDDFAQLNLPDRFKSSFDFIDGYDKPV--- | 224 |

TABLE XXI
Maize soluble starch synthase IIa (SSIIa)
“LINKR” Domain Alignments with other similar proteins

| SEQ | Accession | a.a | | a.a. |
|---|---|---|---|---|
| Id.No. | Number | # | (start) sequence end | # |
| 527 | MaizeSSIIa | 442 | ----G--------------GEHANIFAAGLKMADRVVTVSRGYLWE-L-KT-VEG-GWG | 478 |
| 528 | 7489710 | 442 | ----G--------------GEHANIFAAGLKMADRVVTVSRGYLWE-L-KT-VEG-GWG | 478 |
| 529 | 8953573 | 508 | ----G--------------GEHANYFAAGLKMADQVVTVSPGYLWE-L-KT-VEG-GWG | 544 |
| 530 | 8953571 | 509 | ----G--------------GEHANYFAAGLKMADQVVVVSPGYLWE-L-KT-VEG-GWG | 545 |
| 531 | 5825480 | 509 | ----G--------------GEHANYFAAGLKMADQVVVVSPGYLWE-L-KT-VEG-GWG | 545 |
| 532 | 7529653 | 509 | ----G--------------GEHANYFAAGLKMADQVVVVSPGYLWE-L-KT-VEG-GWG | 545 |
| 533 | 16265834 | 520 | ----G--------------GEHANIFGAGLKMADRVVTVSPGYLWE-L-KT-TEG-GWG | 556 |
| 534 | 7489695 | 201 | ----G--------------GEHANYFAAGLKMADQVVTVSPGYLWE-L-KT-VEG-GWG | 237 |
| 535 | 15384987 | 347 | ----G--------------GEHSNVFAAGLKMADRAVTVSHGYLWE-I-KT-MDG-GWG | 383 |
| 536 | 15028467 | 404 | ----G--------------GEHSNVFAAGLKMADRAVTVSHGYLWE-I-KT-MDG-GWG | 440 |
| 537 | 7489711 | 408 | ----G--------------GDHSNVFAAGLKTALRVVTVSNGYMWE-L-KT-SEG-GWG | 444 |
| 538 | 2833384 | 462 | ----G--------------GEHFNIFAAGLKTADRIVTVSHGYAWE-L-KT-SEG-GWG | 498 |
| 539 | 2129898 | 462 | ----G--------------GEHFNIFAAGLKTALRIVTVSHGYAWE-L-KT-SEG-GWG | 498 |
| 540 | 6467503 | 461 | ----G--------------GDHFNIFAPGLKVADRVVTVSHGYAWE-L-KT-SEG-GWG | 497 |
| 541 | 15232051 | 502 | ----G--------------GEHFNIFAAGLKAADRVLTVSHGYSWE-V-KT-LEG-GWG | 538 |
| 542 | 3192881 | 340 | ----G--------------GDHFNIFAAGLKTADRVVTVSHGYAWE-L-KT-SEG-GWG | 376 |
| 543 | 2833390 | 497 | ----G--------------GEHFNIFAAGLKTADRVVTVSHGYSWE-L-KT-SQG-GWG | 533 |
| 544 | 14495348 | 459 | ----G--------------GDHLNIFAAGIKAADRLLTVSHGYAWE-L-KT-AEG-GWG | 495 |
| 545 | 8708896 | 286 | ----G--------------GEHMNVMKAGLECAHRLVAVSKCYAWE-C-QT-VEG-GWG | 322 |
| 546 | 2833387 | 204 | RHALDK-------------GEAVNFLKGAVVTADRIVTVSQGYSWE-V-TT-AEG-GQG | 245 |
| 547 | 15237934 | 345 | -----GWVFPTWARTHALDTGEAVNLKGAIVTSDRIITVSQGYAWE-I-TT-VEG-GYG | 395 |
| 548 | 5880466 | 350 | RHALDK-------------GEAVNFLKGAVVTADRIVTVSQGYSWE-V-TT-AEG-GQG | 391 |
| 549 | 6690399 | 268 | -----GWVFPTWARTHALDTGEAVNLKGAIVTSDRIITVSQGYAWE-I-TT-VEG-GYG | 318 |
| 550 | 9369334 | 350 | RHALDK-------------GEAVNFLKGAVVTADRIVTVSQGY8WE-V-TT-AEG-GQG | 391 |
| 551 | 6103327 | 350 | RHALDK-------------GEAVNFLKGAVVTADRIVTVSQGYSWE-V-TT-AEG-GQG | 391 |
| 552 | 7188796 | 346 | RHALDK-------------GEAVNFLKGAVVTADRIVTVSQGYSWE-V-TT-AEG-GQG | 387 |
| 553 | 2829792 | 343 | AHALDT-------------GETVNVLKGAIAVADRILTVSQGYSWE-I-TT-PEG-GYG | 384 |
| 554 | 7484400 | 32 | ----G--------------GEHMNVMKAGLECAHRLVAVSKCYAWE-C-QT-VEG-GWG | 68 |
| 555 | 12019656 | 350 | RHALDK-------------GEAVNFLKGAVVTAHPLVTVSKGYSWE-V-TT-AEG-GQG | 391 |
| 556 | 7489712 | 343 | RHALDK-------------GEAVNFLKGAVVTADRIVTVSKGYSWE-V-TT-AEG-GQG | 384 |
| 557 | 2833377 | 344 | RHALDK-------------GEAVNFLKGAVVTADRIVTVSQGYSWE-V-TT-AEG-GQG | 385 |
| 558 | 1549232 | 344 | RHALDK-------------GEAVNFLKGAVVTADRIVTVSQGYSWE-V-TT-AEG-GQG | 385 |
| 559 | 5295947 | 344 | RHALDK-------------GEAVNFLKGAVVTADRIVTVSQGYSWE-V-TT-AEG-GQG | 385 |
| 560 | 6136121 | 300 | -----K-------------GRKINWMKAGILESDRVVTVSPYYAME-L-VSGAEK-GVE | 337 |
| 561 | 5441242 | 298 | -----R-------------GRKINWMKAAILESDLVLTVSPYYAKE-L-VS-GEDRGVE | 335 |
| 562 | 2833388 | 300 | -----K-------------GRKINWMKAGILESDRVLTVSPYYAQEVI-SG-VER-GVE | 337 |
| 563 | 3832512 | 299 | -----K-------------GRKINWMKAGVLESDRVFTVSPYYAKE-L-VS-GEDRGVE | 336 |
| 564 | 15223331 | 302 | -----K-------------GRKINWMKAAILEAHRVLTVSPYYAQE-L-ISGVDR-GVE | 339 |
| 565 | 2833383 | 295 | -----E-------------GKKINWMKAGILESDQVFTVSPHYAKE-L-IS-GEDRGVE | 332 |
| 566 | 15637079 | 300 | -----K-------------GRKINWMKAGIREADRVFTVSPNYAKE-L-VSCVSK-GVE | 337 |
| 567 | 267196 | 299 | -----K-------------GRKINWMKAGILESHRVVTVSPYYAQE-LVSA-VDK-GVE | 336 |
| 568 | 228210 | 299 | -----K-------------GRKINWMKAGILESHRVVTVSPYYAQE-LVSA-VDK-GVE | 336 |
| 569 | 602594 | 299 | -----K-------------GRKINWMKAGILESHRVVTVSPYYAQE-L-VSAVDK-GVE | 336 |
| 570 | 2833381 | 300 | -----K-------------GRKINWMKAGIREADRVFTVSPNYAKE-L-VSCVSK-GVE | 337 |
| 571 | 12003285 | 296 | -----K-------------GRKINWMKAGIIESDRVLTVSPYYAKE-L-VSGPDK-GVE | 333 |
| 572 | 15626365 | 305 | -----V-------------GRKINWMkAGIIESHRVLTVSPYYAQE-L-VSGPDK-GVE | 342 |
| 573 | 6624281 | 295 | -----E-------------GRKINWMKAGILQADKVLTVSPYYAEE-L-IS-GEARGCG | 332 |
| 574 | 6624287 | 295 | -----E-------------GRKINWMKAGILQADKVLTVSPYYAEE-L-IS-GEARGCE | 332 |
| 575 | 4760584 | 295 | -----E-------------GRKINWMKAGILQADKVLTVSPYYAEE-L-IS-GEARGCE | 332 |
| 576 | 18139611 | 299 | -----V-------------GRKINWMKAGITECDLVMTVSPHYVKE-L-ASGPDK-GVE | 336 |
| 577 | 17736918 | 225 | -----E-------------GRKINWMKAGILQADKVLTVSPYYAEE-L-IS-GETRGCE | 262 |
| 527 | Maize SSIIa | 479 | LHDIIRSNDWKINGIVNGIDHQEWNPKVDVHL-RSDGYTN--YSLETLDAGKRQCKAALQ | 535 |
| 528 | 7489710 | 479 | LHDIIRSNDWKINGIVNGIDHQEWNPKVDVHL-RSDGYTN--YSLETLDAGKRQCKAALQ | 535 |
| 529 | 8953573 | 545 | LHDIIRQNDWKTRGIVNGIDNMEWNPEVDVHL-KSDGYTN--FSLGTLDSGKRQCKEALQ | 601 |
| 530 | 8953571 | 546 | LHDIIRQNDWKTRGIVNGIDNMEWNPEVDVHL-QSDGYTN--FSLSTLDSGKRQCKEALQ | 602 |
| 531 | 5825480 | 546 | LHDIIRQNDWKTRGIVNGIDNMEWNPEVDVHL-KSDGYTN--FSLGTLDSGKRQCKEALQ | 602 |
| 532 | 7529653 | 546 | LHDIIRQNDWKTRGIVNGIDNMEWNPEVDVHL-KSDGYTN--FSLGTLDSGKRQCKEALQ | 602 |
| 533 | 16265834 | 557 | LHDIIRENDWKMNGIVNGIDYREWNPEVDVHL-QSDGYAN--YTVASLDSSKPRCKAALQ | 613 |
| 534 | 7489695 | 238 | LHDIIRQNDWKTRGIVNGIDNMEWNPEVDVHL-KSDGYTN--FSLGTLDSGKRQCKEALQ | 294 |
| 535 | 15384987 | 384 | LHEIINHNDWKLQGIVNGIDMAEWNPEVDEHL-QSDGYAN--YTFETLDTGKKQCKEALQ | 440 |
| 536 | 15028467 | 441 | LHEIINHNDWKLQGIVNGIDMAEWNPEVDEHL-QSDGYAN--YTFETLDTGKKQCKEALQ | 497 |
| 537 | 7489711 | 445 | LHDIINQNDWKLQGIVNGIDMSEWNPAVDVHL-HSDDYTN--YTFETLDTGKRQCKAALQ | 501 |
| 538 | 2833384 | 499 | LHNIINESDWKFRGIVNGVDTKDWNPQFDAYL-TSDGYTN--YNLKTLQTGKRQCKAALQ | 555 |
| 539 | 2129898 | 499 | LHNIINESDWKFRGIVNGVDTKDWNPQFDAYL-TSDGYTN--YNLKTLQTGKRQCKAALQ | 555 |
| 540 | 6467503 | 498 | LHNIINENHWKLQGIVNGIDAKEWNPQFDIQL-TSDGYTN--YSLETLDTGKPQCKTALQ | 554 |
| 541 | 15232051 | 539 | LHNIINENDWKFRGIVNGIDTQEWNPEFDTYL-HSDDYTN--YSLENLHIGKPQCKAALQ | 595 |
| 542 | 3192881 | 377 | LNGIRNENEWKLQGIVNGIDIEEWNPQLDVYL-KSDGYAN--YSLDTLQTGKPQCKAALQ | 433 |
| 543 | 2833390 | 534 | LHQIINENDWKLQGIVNGIDTKEWNPELDVHLPRSDGYMN--YSLDTLQTGKPQCKAALQ | 591 |
| 544 | 14495348 | 496 | LHGIINESDWKFQGIVNGIDTTDWNPRCDIHL-KSDGYTN--YSLETVQAGKQQCKAALQ | 552 |
| 545 | 8708896 | 323 | LHEVIKVNNWKLRGIVNGIDYKEWNPICDEFL-TTDGYAH--YDVDTLAEGKAKCKAALQ | 379 |
| 546 | 2833387 | 246 | LNELLSSRKSVLNGIVNGIDINDWNPTTDKCL-PH----H--YSVDDL-SGKAKCKAELQ | 297 |
| 547 | 15237934 | 396 | LQDLLSSRKSVINGITNGINVDEWNFSTDEHI-P----FH--YSADDV-SEKIKCKNALQ | 447 |
| 548 | 5880466 | 392 | LNELLSSRKSVLNGIVNGIDINDWNPTTDKCL-PH----H--YSVDDL-SGKAKCKAELQ | 443 |
| 549 | 6690399 | 319 | LQDLLSSRKSVINGITNGINVDEWNPSTDEHI-P----FH--YSADDV-SEKIKCKAELQ | 370 |
| 550 | 9369334 | 392 | LNELLSSRKSVLNGIVNGIDINDWNPTTDKCL-PH----H--YSVDDL-SGKAKCKAELQ | 443 |
| 551 | 6103327 | 392 | LNELLSSRKSVLNGIVNGIDINDWNPTTDKCL-PH----H--YSVDDL-SGKAKCKAELQ | 443 |
| 552 | 7188796 | 388 | LNELLSSRKSVLNGIVNGIDINDWNPTTDKCL-PH----H--YSVDDL-SGKAKCKAELQ | 439 |
| 553 | 2829792 | 385 | LHELLSSRQSVLNGITNGIDVNDWNPSTDEHI-AS----H--YSINDL-SGKVQCKTDLQ | 436 |
| 554 | 7484400 | 69 | LHEVIKVNNWKLRGIVNGIDYKEWNPICDEFL-TTDGYAH--YDVDTLAEGKAKCKAALQ | 125 |
| 555 | 12019656 | 392 | LNELLSSRKSVLNGIVNGIDINDWNPATDKCI-P----CH--YSVDDL-SGKAKCKGALQ | 443 |
| 556 | 7489712 | 385 | LNELLSSRKSVLNGIVNGIDINDWNPATDKCI-P----CH--YSVDDL-SGKAKCKGALQ | 436 |
| 557 | 2833377 | 386 | LNELLSSRKSVLNGIVNGIDINDWNPSTDKFL-P----YH--YSVDDL-SGKAKCKAELQ | 437 |
| 558 | 1549232 | 386 | LNELLSSRKSVLNGIVNGIDINDWNPSTDKFL-P----YH--YSVDDL-SGKAKCKAELQ | 437 |
| 559 | 5295947 | 386 | LNELLSSRKSVLNGIVNGIDINDWNPSTDKFL-P----YH--YSVDDL-SGKAKCKAELQ | 437 |
| 560 | 6136121 | 338 | LDNVIAKT--SITGIVNGMDTQEWNPATDKHI-D----TN--YDITTVMDAKPLLKEALQ | 388 |
| 561 | 5441242 | 336 | LDNIIRKTG--VAGIVNGMDIREWSPKTDKFI-D----IH--FDTTSVKEAKFLLKEALQ | 386 |
| 562 | 2833388 | 338 | LDNFIRKTG--IAGIIHGMDVQEWNPVTDKYI-D----IH--YDATTVMDAKPLLKEALQ | 388 |
| 563 | 3832512 | 337 | LDNIIRS--IGITGIVNGMDNREWSPQTDRYI-D----VH--YDASTVTEAKAILKEALQ | 387 |
| 564 | 15223331 | 340 | LHKYLRMK--TVSGIINGMDVQEWNPSTDKYI-D----IK--YDITTVTDAKPLIKEALQ | 390 |
| 565 | 2833383 | 333 | LDNIIRSTG--IIGIVNGMDNREWSPQTDRYI-D----VH--YNETTVTEAKPLLKGTLQ | 383 |
| 566 | 15637079 | 338 | LDNHIR--DCGITGICNGMLTQEWNPATDKYL-A----VK--YDITTVMQAKPLLKEALQ | 388 |
| 567 | 267196 | 337 | LDSVLRKT--CITGIVNGMDTQEWNPA-------TDKYTDVKYDITTVMDAKPLLKEALQ | 387 |
| 568 | 228210 | 337 | LDSVLRKT--CITGIVNGMDTQEWNPA-------TDKYTDVKYDITTVMDAKPLLKEALQ | 387 |
| 569 | 602594 | 337 | LDSVLRKT--CITGIVNGMDTQEWNPA-------TDKYTDVKYDITTVMDAKPLLKEALQ | 387 |
| 570 | 2833381 | 338 | LDNHIR--DCGITGICNGMDTQEWNPATDKYL-A----VK--YDITTVMQAKPLLKEALQ | 388 |
| 571 | 12003285 | 334 | LDNILRK--CTVTGIVNGMDTQEWNPATDKYI-DN----H--YDITTVMDGKPLLKEALQ | 384 |
| 572 | 15626365 | 343 | LDNILRRVG--VTGIVNGMDVQEWNPSTDKYI-S----IK--YDASTVLEGKALLKEELQ | 393 |
| 573 | 6624281 | 333 | LDNIMRLTG--ITGIVNGMDVSEWDPIKDKFL-T----VN--YDVTTALEGKALNKEALQ | 383 |
| 574 | 6624287 | 333 | LDNIMRLTG--ITGIVNGMLVSEWDPTKDKFL-A----VN--YDVTTALEGKALNKEALQ | 383 |
| 575 | 4760584 | 333 | LDNIMRLTG--ITGIVNGMDVSEWDPTKDKFL-A----VN--YDITTALEGKALNKEALQ | 383 |
| 576 | 18139611 | 337 | LDGILRTKPLE-TGIVNGMDVYEWNPATDQYI-S----VK--YDATTVTEARALNKEMLQ | 388 |
| 577 | 17736918 | 263 | LDNIMRLTG--ITGIVNGNDVSEWDPTKDKFL-A----VN--YDITTALEGKALNKEALQ | 313 |

TABLE XXII
Maize soluble starch synthase IIa (SSIIa)
“GLYTR” Domain Alignments with other similar proteins

| SEQ | Accession | a.a | | a.a. |
|---|---|---|---|---|
| Id.No. | Number | # | (start) Sequence end | # |
| 578 | MaizeSSIIa | 536 | RELGLEVRDDVPLLGFIGRLDGQKGVDIIGDAMPWI-AG-QDVQLVMLGTGRADLERMLQ | 593 |
| 579 | 7489710 | 536 | RELGLEVRDDVPLLGFIGRLDGQKGVDIIGDAMPWI-AG-QDVQLVMLGTGRADLERMLQ | 593 |
| 580 | 8953571 | 602 | RELGLQVRGDVPLLGFIGRLDGQKGVEIIADAMPWI-VS-QDVQLVMLGTGPHDLEGMLR | 659 |
| 581 | 8953571 | 603 | RELGLQVRADVPLLGFIGRLDGQKGVEIIADAMPWI-VS-QDVQLVMLGTGRHDLESMLR | 660 |
| 582 | 5825480 | 603 | RELGLQVRADVPLLGFIGRLDGQKGVEIIADAMPWI-VS-QDVQLVMLGTGRHDLESMLR | 660 |
| 583 | 7529653 | 603 | RELGLQVRADVPLLGFIGRLDGQKGVEIIADAMPWI-VS-QDVQLVMLGTGRHDLESMLQ | 660 |
| 584 | 16265834 | 614 | RELGLERVDDVPLIGFIGRLDGQKGVDIIGDAMPWI-AG-QDVQLVLLGSGRRDLEVMLQ | 671 |
| 585 | 7489695 | 295 | RELGLQVRADVPLLGFIGRLDGQKGVEIIADAMPWI-VS-QDVQLVMLGTGRHDLESMLQ | 352 |
| 586 | 15384987 | 441 | RQLGLQVRDDVPLIGFIGRLDHQKGVDIIGDAMPWI-AG-QDVQVVMLGTGRPDLEEMLR | 498 |
| 587 | 15028467 | 498 | RQLGLQVRDDVPLIGFIGRLDHQKGVDIIGDAMPWI-AG-QDVQVVMLGTGRPDLEEMLR | 555 |
| 588 | 7489711 | 502 | RQLGLQVRDDVPLIGFIGRLDHQKGVDIIADAIHWI-AG-QDVQLVMLGTGRADLEDMLR | 559 |
| 589 | 2833384 | 556 | RELGLPVREDVPIISFIGRLDHQKGVDLIAEAIPWM-MS-HDVQLVMLGTGRADLEQMLK | 613 |
| 590 | 2129898 | 556 | RELGLPVREDVPIISFIGRLDHQKGVDLIAEAIPWM-MS-HDVQLVMLGTGRADLEQMLK | 613 |
| 591 | 6467503 | 555 | NELRFAIPPDVPVIGFIGRLDYQKGVDLIAEAIPWM-VG-QDVQLVMLGTGRQDLEEMLR | 612 |
| 592 | 15232051 | 596 | KELGLPVRPDVPLIGFIGRLDHQKGVDLIAEAVPWM-MS-QDVQLVMLGTGRPDLEEVLR | 653 |
| 593 | 3192881 | 434 | KEMNLPVRDDVPLIGFIGRLDHQKGVDLIAEAIPWM-MG-QDVQLVMLGTGRPDLEQMLK | 491 |
| 594 | 2833390 | 592 | KELGLPVRDDVPLIGFIGRLDPQKGVDLIAEAVPWM-MG-QDVQLVMLGTGRPDLEQMLR | 649 |
| 595 | 14495348 | 553 | KELGLPVRGDVPVIAFIGRLDHQKGVDLIAEAMPWI-AG-QDVQLIMLGTGRQDLEDTLR | 610 |
| 596 | 8708896 | 380 | KELGLPVDPDAPMLGFIGRLDYQKGVDLIRDNYDYI-MG-EKCQLVMLGSGRQDLEDALR | 437 |
| 597 | 2833387 | 298 | KELGLPVREDVPLIGFIGRLDYQKGIDLIKMAIPEL-MR-EDVQFVMLGSGDPIFEGWMR | 355 |
| 598 | 15237934 | 448 | KELGLPIRPECPMIGFIGRLDYQKGIDLIQTAGPDL-MV-DDIQFVMLGSGDPKYESWMR | 505 |
| 599 | 5880466 | 444 | KELGLPVREDVPLIGFIGRLDYQKGIDLIKMAIPEL-MR-EDVQFVMLGSGDPIFEGWMR | 501 |
| 600 | 6690399 | 371 | KELGLPIRPECPMIGFIGRLDYQKGIDLIQTAGPDL-MV-DDIQFVMLGSGDPKYESWMR | 428 |
| 601 | 9369334 | 444 | KELGLPVREDVPLIGFIGRLDYQKGIDLIKMAIPEL-MR-EDVQFVMLGSGDPIFEGWMR | 501 |
| 602 | 6103327 | 444 | KELGLPVREDVPLIGFIGRLDYQKGIDLIKMAIPEL-MR-EDVQFVMLGSGDPIFEGWMR | 501 |
| 603 | 7188796 | 440 | RELGLPVREDVPLIGFIGRLDYQKGIDLIKMAIPDL-MR-EDVQFVMLGSGDPVFEGWMR | 497 |
| 604 | 2829792 | 437 | KELGLPIRPDCPLIGFIGRLDYQKGVDIILSAIPEL-MQ-NDVQVVMLGSGEKQYEDWMR | 494 |
| 605 | 7484400 | 126 | KELGLPVDPDAPMLGFIGRLDYQKGVDLIRDNYDYI-MG-EKCQLVMLGSGRQDLEDALR | 183 |
| 606 | 12019656 | 444 | KELGLPIRPEVPLIGFIGRLDYQKGIDLIQLIIPHL-MR-DDVQFVMLGSGDPELEDWMR | 501 |
| 607 | 7489712 | 437 | KELGLPIRPDVPLIGFIGRLDYQKGIDLIQLIIPDL-MR-EDVQFVMLGSGDPELEDWMR | 494 |
| 608 | 2833377 | 438 | KELGLPIRPDVPLIGFIGRLDYQKGIDLIKLAIPDL-MR-DNIQFVMLGSGDPGFEGWMR | 495 |
| 609 | 1549232 | 438 | KELGLPIRPDVPLIGFIGRLDYQKGIDLIKLAIPDL-MR-DNIQFVMLGSGDPGFEGWMR | 495 |
| 610 | 5295947 | 438 | KELGLPIRPDVPLIGFIGRLDYQKGIDLIKLAIPDL-MR-DNIQFVMLGSGDPGFEGWMR | 495 |
| 611 | 6136121 | 389 | AAVGLPVDKNIPVIGFIGRLEEQKGSDILVAAISKF-VG-LDVQIIILGTGKKKFEQQIQ | 446 |
| 612 | 5441242 | 387 | AEVGLPVNRDIPLIGFIGRLEEQKGSDILVEAIPKF-ID-QNVQIIILGTGKKSMEKQIE | 444 |
| 613 | 2833388 | 389 | AEVGLPVDRNVPLIGFIGRLEEQKGSDIFVAAISQL-VE-HNVQIVILGTGKKKFEKQIE | 446 |
| 614 | 3832512 | 388 | AEVGLPVDRNIPVIGFIGRLEEQKGSDILVESIPKF-ID-QNVQIIVLGTGKKIMEKQIE | 445 |
| 615 | 15223331 | 391 | AAVGLPVDRDVPVIGFIGRLEEQKGSDILVEAISKF-MG-LNVQMVILGTGKKKMEAQIL | 448 |
| 616 | 2833383 | 384 | AEIGLPVDSSIPLIGFIGRLEEQKGSDILVEAIAKF-AD-ENVQIVVLGTGKKIMEKQIE | 441 |
| 617 | 15637079 | 389 | AAVGLPVDRNIFLIGFIGRLEEQKGSDILYAAISKF-IS-MDVQILILGTGKKKFEQQIE | 446 |
| 618 | 297196 | 388 | AAVGLPVDKKIPLIGFIGRLEEQKGSDILVAAIHKF-IG-LDVQIVVLGTGKKEFEQEIE | 445 |
| 619 | 228210 | 388 | AAVGLPVDKKIPLIGFIGRLEEQKGSDILVAAIHKF-IG-LDVQIVVLGTGKKEFEQEIE | 445 |
| 620 | 602594 | 388 | AAVGLPVDKKVPLIGFIGRLEEQKGSDILVAAIHKF-IG-LDVQIVVLGTGKKEFEQEIE | 445 |
| 621 | 2833381 | 389 | AAVGLPVDRNIPLIGFIGRLEEQKGSDILYAAISKF-IS-MDVQILILGTGKKKFEQQIE | 446 |
| 622 | 12003285 | 385 | AEVGLPVDP VPLVGFIGRLEEQKGSDILVAALHKF-IE-MDVQVVILGTGKKEFEKQIE | 442 |
| 623 | 15626365 | 394 | AEVGLPVDKNVPLIAFIGRLEEQKGSDILVEAIPQF-IK-ENVQIVALGTGKKEMEKQLQ | 451 |
| 624 | 6624281 | 384 | AEVGLPVDRKVPLVAFIGRLEEQKGPDVMIAAIPEI-VKEEDVQIVLLGTGKKKFERLLK | 442 |
| 625 | 6624287 | 384 | AEVGLPVDRKVPLVAFIGRLEEQKGPDVMIAAIPEILKE-EDVQIVLLGTGKKKFERLLK | 442 |
| 626 | 4760584 | 384 | AEVGLPVDRKVPLVAFIGRLEEQKGPDVMIAAIPEILKE-EDVQIVLLGTGKKKFERLLK | 442 |
| 627 | 18139611 | 389 | AEVGLPVDSSIPLIVFVGRLEEQKGSDILIAAIPEF-VE-GNVQIIVLGTGKKKMEEELI | 446 |
| 628 | 17736918 | 314 | AEVGLPVDRKVPLVAFIGRLEEQKGPDVMIAAIPEILKE-EDVQIVLLGTGKKKFERLLK | 372 |
| 578 | MaizeSSIIa | 594 | HLEREHPNKVRGWVGFSVPMAHRITAGADVLVMPSRFEPCGLNQLYAMAYGTVPVVHAVG | 653 |
| 579 | 7489710 | 594 | HLEREHPNKVRGWVGFSVPMAHRITAGADVLVMPSRFEPCGLNQLYAMAYGTVPVVHAVG | 653 |
| 580 | 8953571 | 660 | HFEREHHDKVRGWVGFSVRLAHRITAGADALLMPSRFEPCGLNQLYAMAYGTVPVVHAVG | 719 |
| 581 | 8953571 | 661 | HFEREHHDKVRGWVGFSVRLAHRITAGADALLMPSRFEPCGLNQLYAMAYGTVPVVHAVG | 720 |
| 582 | 5825480 | 661 | HFEREHHDKVRGWVGFSVRLAHRITAGADALLMPSRFEPCGLNQLYAMAYGTVPVVHAVG | 720 |
| 583 | 7529653 | 661 | HFEREHHDKVRGWVGFSVRLAHRITAGADALLMPSRFEPCGLNQLYAMAYGTVPVVHAVG | 720 |
| 584 | 16265834 | 672 | RFEAQHNSKVRGWVGFSVKMAHRITAGADVLVMPSRFEPCGLNQLYAMAYGTVPVVHAVG | 731 |
| 585 | 7489695 | 353 | HFEREHHDKVRGWVGFSVRLAHRITAGADALLMPSRFEPCGLNQLYAMAYGTVPVVHAVG | 412 |
| 586 | 15384987 | 499 | RFESEHNDKVRGWVGFSVQLAHRITAGADVLLMPSRFEPCGLNQLYAMAYGTVPVVHAVG | 558 |
| 587 | 15028467 | 556 | RFESEHNDKVRGWVGFSVQLAHRITAGADVLLMPSRFEPCGLNQLYAMAYSTVPVVHAVG | 615 |
| 588 | 7489711 | 560 | RFESEHSDKVRAWVGFSVPLAHRITAGADILLMPSRFEPCGLNQLYAMAYGTVPVVHAVG | 619 |
| 589 | 2833384 | 614 | EFEAQHCDKIRSWVGFSVKMAHRITAGSDILLMPSRFEPCGLNQLYAMSYGTVPVVHGVG | 673 |
| 590 | 2129898 | 614 | EFEAQHCDKIRSWVGFSVKMAHRITAGSDILLMPSRFEPCGLNQLYAMSYGTVPVVHGVG | 673 |
| 591 | 6467503 | 613 | QFENQHRDKVRGWVGFSVKTAHRITAGADILLMPSRFEPCGLNQLYAMMYGTIPVVHAVG | 672 |
| 592 | 15232051 | 654 | QMEHQYRDKARGWVGFSVKTAHRITAGADILLMPSRFEPCGLNQLYAMNYGTIPVVHAVG | 713 |
| 593 | 3192881 | 492 | QIEGQYGDKVRGWVGFSVKTAHRITAGADILLMPSRFEPCGLNQLYAMSYGTVPVVHAVG | 551 |
| 594 | 2833390 | 650 | QFECQHNDKIRGWVGFSVKTSHRITAGADILLMPSRFEPCALNQLYAMKYGTIPVVHAVG | 709 |
| 595 | 14495348 | 611 | RLESQHYDRVRGWVGFSIRLAHRMTAGADILLMPSRFEPCGLNQLYAMMYGTVPVVHAVG | 670 |
| 596 | 8708896 | 438 | DMENRNKNQCRGWVGFSNKMAHRITAAADILLMPSRFEPCGLNQLYAMAYGTVPIVHSVG | 497 |
| 597 | 2833387 | 356 | STESSYKDKFRGWVGFSVPVSHRITAGCDILLMPSRFEPCGLNQLYAMQYGTVPVVHGTG | 415 |
| 598 | 15237934 | 506 | SMEETYRDKFRGWVGFNVPISHRITAGCDILLMPSRFEPCGLNQLYAMRYGTIPVVHGTG | 565 |
| 599 | 5880466 | 502 | STESSYKDKFRGWVGFSVPVSHRITAGCDILLMPSRFEPCGLNQLYAMQYGTVPVVHGTG | 561 |
| 600 | 6690399 | 429 | SMEETYRDKFRGWVGFNVPISHRITAGCDILLMPSRFEPCGLNQLYAMRYGTIPVVHGTG | 488 |
| 601 | 9369334 | 502 | STESSYKDKFRGWVGFSVPVSHRITAGCDILLMPSRFEPCGLNQLYAMQYGTVPVVHGTG | 561 |
| 602 | 6103327 | 502 | STESSYKDKFRGWVGFSVPVSHRITAGCDILLMPSRFEPCGLNQLYAMQYGTVPVVHGTG | 561 |
| 603 | 7188796 | 498 | STESSYKDKFRGWVGFSVPVSHRITAGCDILLMPSRFEPCGLNQLYAMQYGTVPVVHGTG | 557 |
| 604 | 2829792 | 495 | HTENLFKDKFRAWVGFNVPVSHRITAGCDILLMPSRFEPCGLNQLYAMRYGTIPIVHSTG | 554 |
| 605 | 7484400 | 184 | DMENRNKNQCRGWVGFSNKMAHRITAAADILLMPSRFEPCGLNQLYAMAYGTVPIVHSVG | 243 |
| 606 | 12019656 | 502 | STESDFKDKFRGWVGFSVPVSHRITAGCDILLMPSRFEPCGLNQLYAMQYGTVPVVHATG | 561 |
| 607 | 7489712 | 495 | STESIFKDKFRGWVGFSVPVSHRITAGCDILLMPSRFEPCGLNQLYAMQYGTVPVVHATG | 554 |
| 608 | 2833377 | 496 | STESGYRDKFRGWVGFSVPVSHRITAGCDILLMPSRFEPCGLNQLYAMQYGTVPVVHGTG | 555 |
| 609 | 1549232 | 496 | STESGYRDKFRGWVGFSVPVSHRITAGCDILLMPSRFEPCGLNQLYAMQYGTVPVVHGTG | 555 |
| 610 | 5295947 | 496 | STESGYRDKFRGWVGFSVPVSHRITAGCDILLMPSRFEPCGLNQLYAMQYGTVPVVHGTG | 555 |
| 611 | 6136121 | 447 | ELEVLYPDKARGVAKFNVPLAHMITAGADFMLVPSRFEPCGLIQLHAMRYGTIPICASTG | 506 |
| 612 | 5441242 | 445 | QLEEIYPEKARGIAKFDGPLAHKIIAGSDFIMIPSRFEPCGLVQLHSMPYGTVPIVSSTG | 504 |
| 613 | 2833388 | 447 | HLEVLYPDKARGVAKFNVPLAHMITAGADFMLVPSRFEPCGLIQLHAMRYGTVPIVASTG | 506 |
| 614 | 3832512 | 446 | QLEVTYPGKAIGVAKFNSPLAHKIIAGADFIVIPSRFEPCGLVQLHAMPYGTVPIVSSTG | 505 |
| 615 | 15223331 | 449 | ELEEKFPGKAVGVAKFNVPLAHMITAGADFIIVPSRFEPCGLIQLHAMRYGTVPIVASTG | 508 |
| 616 | 2833383 | 442 | VLEEKYPGKAIGITKFNSPLAHKIIAGADFIVIPSRFEPCGLVQLHAMPYGTVPIVSSTG | 501 |
| 617 | 15637079 | 447 | QLEVMYPDKARGVAKFNVPLAHMITAGADFMLIPSRFEPCGLIQLHAMRYGTPCICASTG | 506 |
| 618 | 297196 | 446 | QLEVLYPNKAKGVAKFNVPLAHMITAGADFMLVPSRFEPCGLIQLHAMRYGTVPICASTG | 505 |
| 619 | 228210 | 446 | QLEVLYPGKVKGVAKFNVPLAHMITAGADFMLVPSRFEPCGLIQLHAMRYGTVPICASTG | 505 |
| 620 | 602594 | 446 | QLEVLYPNKAKGVAKFNVPLAHMITAGADFMLVPSRFEPCGLIQLHAMRYGTVPICASTG | 505 |
| 621 | 2833381 | 447 | QLEVMYPDKARGVAKFNVPLAHMITAGADFMLIPSRFEPCGLIQLHAMRYGTPCICASTG | 506 |
| 622 | 12003285 | 443 | QLEELYPGKAVGVAKFNVPLAHKITAGADFMLVPSRFEPCGLIQLHAMRYGTIPICASTG | 502 |
| 623 | 15626365 | 452 | QLEISYPDKARGVAKFNVPLAHMMIAGADFILIPSRFEPCGLIQLQAMRYGTVPIVASTG | 511 |
| 624 | 6624281 | 443 | SVEEKFPTKVRAVVRFNAPLAHQMMAGADVLAVTSRFEPCGLIQLQGMRYGTPCACASTG | 502 |
| 625 | 6624287 | 443 | SVEEKFPNKVRAVVRFNAPLAHQMMAGADVLAVTSRFEPCGLIQLQGMRYGTPCACASTG | 502 |
| 626 | 4760584 | 443 | SIEEKFPSKVRAVVRFNAPLAHQMMAGADVLAVTSRFEPCGLIQLQGMRYGTPCACASTG | 502 |
| 627 | 18139611 | 447 | LLEVKYPNTARGLAKFNVPLAHMMFAGADFIIVPSRFEPCGLIQLQGMRYGVVPICSSTG | 506 |
| 628 | 17736918 | 373 | SIEEKFPSKVRAVVRFNAPLAHQMMAGADVLAVTSRFEPCGLIQLQGMRYGTPCACASTG | 432 |
| 578 | MaizeSSIIa | 654 | GLRDTV---------AP-FDPF----GDAGL----G---W--------TFDR---AEANK | 681 |
| 579 | 7489710 | 654 | GLRDTV---------AP-FDPF----GDAGL----G---W--------TFDR---AEANK | 681 |
| 580 | 8953571 | 720 | GLRDTV---------PP-FDPF----NHSGL----G---W--------TFDR---AEAQK | 747 |
| 581 | 8953571 | 721 | GLRDTV---------PP-FDPE----NHSGL----G---W--------TFDR---AEAHK | 748 |
| 582 | 5825480 | 721 | GVRDTV---------PP-FDPF----NHSGL----G---W--------TFDR---AEAHK | 748 |
| 583 | 7529653 | 721 | GLRDTV---------PP-FDPF----NHSGL----G---W--------TFDR---AEAHK | 748 |
| 584 | 16265834 | 732 | GLRDTM---------SA-FDPF----EDTGL----G---W--------TFDR---AEPHK | 759 |
| 585 | 7489695 | 413 | GLRDTV---------PP-FDPF----NHSGL----G---W--------TFDR---AEAHK | 440 |
| 586 | 15384987 | 559 | GLRDTV---------AP-FDPF----ADTGL----G---W--------TFDR---AEANR | 586 |
| 587 | 15028467 | 616 | GLRDTV---------AP-FDPF----ADTGL----G---W--------TFDR---AEANR | 643 |
| 588 | 7489711 | 620 | GLRDTV---------AP-FDPF----NDTGL----G---W--------TFDR---AEANR | 647 |
| 589 | 2833384 | 674 | GLRDTV---------QP-FNPF----DESGV----G---W--------TFDR---AEANK | 701 |
| 590 | 2129898 | 674 | GLRDTV---------QP-FNPF----DESGV----G---W--------TFDR---AEANK | 701 |
| 591 | 6467503 | 673 | GLRDTV---------QP-FDPF----NESGL----G---W--------TFDS---AESHK | 700 |
| 592 | 15232051 | 714 | GLRDTV---------QQ-FDPY----SETGL----G---W--------TFDS---AEAGK | 741 |
| 593 | 3192881 | 552 | GLRDTV---------QP-FDPF----NESGY----G---W--------TFGR---ARANQ | 579 |
| 594 | 2833390 | 710 | GLRDTV---------QP-FDPL----MSQDW----G---G--------PSDR---AEASQ | 737 |
| 595 | 14495348 | 671 | GLRDTV---------EH-YNPY----EESGL----G---W--------TFEK---AEANR | 698 |
| 596 | 8708896 | 498 | GLRDTV---------KQ-YSPF----ENVGT----G---W--------VFER---AEANK | 525 |
| 597 | 2833387 | 416 | GLRDTV---------ET-FNPFGAK-GEEGT----G---W--------AFSP---LTVDK | 446 |
| 598 | 15237934 | 566 | GLRDTV---------EN-FNPYAEGGAGTGT----G---W--------VFTP---LSKDS | 597 |
| 599 | 5880466 | 562 | GLRDTV---------ET-FNPFGAK-GEEGT----G---W--------AFSP---LTVDK | 592 |
| 600 | 6690399 | 489 | GLRDTV---------EN-FNFYAEGGAGAGT----c---W--------VFTP---LSKDS | 520 |
| 601 | 9369334 | 562 | GLRDTV---------ET-FNPFGAK-GEEGT----G---W--------AFSP---LTVDK | 592 |
| 602 | 6103327 | 562 | GLRDTV---------ET-FNPFGAK-GEEGT----G---W--------AFSP---LTVDK | 592 |
| 603 | 7188796 | 558 | GLRDTV---------ET-FNPFGAK-GEEGT----G---W--------AFSP---LTVEK | 588 |
| 604 | 2829792 | 555 | GLRDTV---------KD-FNPY----AQEGIGEGTG---W--------TFSP---LTSEK | 586 |
| 605 | 7484400 | 244 | GLRDTV---------KQ-YSPF----ENVGT----G---W--------VFER---AEANK | 271 |
| 606 | 12019656 | 562 | GLRDTV---------EN-FNPFGEN-GEQGT----G---W--------AFAP---LTTEN | 592 |
| 607 | 7489712 | 555 | GLRDTV---------EN-FNPFGEN-GEQGT----G---W--------AFAP---LTTEN | 585 |
| 608 | 2833377 | 556 | GLRDTV---------EN-FNPFAEK-GEQGT----G---W--------AF | 579 |
| 609 | 1549232 | 556 | GLRDTV---------EN-FNPFAEK-GEQGT----G---W--------AF | 579 |
| 610 | 5295947 | 556 | GLRDTV---------EN-FNPFAEK-GEQGT----G---W--------AF | 579 |
| 611 | 6136121 | 507 | GLVDTV---------TEGFTGF----HMGAF----NVECA--------TVDP---ADVQK | 538 |
| 612 | 5441242 | 505 | GLVDTV---------QEGFTGF----HMGAF----NVDCE--------AIDP---ADVEK | 536 |
| 613 | 2833388 | 507 | GLVDTV---------KEGYTGF----QMGAL----H---V--------ECDKIDSADVAA | 538 |
| 614 | 3832512 | 506 | GLVDTV---------KEGYTGF----HVGAF----SVECE--------AVDP---ADVEK | 537 |
| 615 | 15223331 | 509 | GLVDTV---------KDGYTGF----HIGRF----N---V--------KCEV---VDPDD | 537 |
| 616 | 2833383 | 502 | GLVDTVKEGYTGFHAGP-FDVE----CE--------------------DVDP---DDVDK | 533 |
| 617 | 15637079 | 507 | GLVDTV---------KEGYTGF----HMGAF----NVDCE--------TVDP---EDVLK | 538 |
| 618 | 297196 | 506 | GLVDTV---------KEGYTGF----HMGAF----N---VECD-----VVDP---ADVLK | 537 |
| 619 | 228210 | 506 | GLVDTV---------KEGYTGF----HMGAF----N---VECD-----VVDP---ADVLK | 537 |
| 620 | 602594 | 506 | GLVDTV---------KEGYTGF----HMGAF----N---VECD-----VVDP---ADVLK | 537 |
| 621 | 2833381 | 507 | GLVDTV---------KEGYTGF----HMGAF----NVDCE--------TVDP---EDVLK | 538 |
| 622 | 12003285 | 503 | GLVDTV---------KEGFTGF----HMGAF----N---VECD-----AVDP---ADVLK | 534 |
| 623 | 15626365 | 512 | GLVDTV---------KEGFTGF----HMGSF----N---V--------KCDA---VDPVD | 540 |
| 624 | 6624281 | 503 | GLVDTI---------VE---------GKTGF----H---MGRLSVDCNVVEP---ADVKK | 534 |
| 625 | 6624287 | 503 | GLVDTI---------VE---------GKTGF----H---MGRLSVDCNVVEP---ADVKK | 534 |
| 626 | 4760584 | 503 | GLVDTI---------VE---------GKTGF----H---MGRLSVDCNVVEP---ADVKK | 534 |
| 627 | 18139611 | 507 | GLVDTV---------KE-GVTG----FHMGLFNVEC---E--------TVDP---VDVTA | 538 |
| 628 | 17736918 | 433 | GLVDTI---------VE---------GKTGF----H---MGRLSVDCNVVEP---ADVKK | 464 |

TABLE XXIII
Maize soluble starch synthase IIa (SSIIa)
“GLYTR” Domain Alignments with other similar proteins

| SEQ | Accession | a.a | | a.a. |
|---|---|---|---|---|
| Id.No. | Number | # | (start) Sequence end | # |
| 629 | MaizeSSIIa | 682 | LIEALRHCLDTYRKYGES-WKSLQARGMSQDLSWDHAAELYEDVLVKAKYQW | 732 |
| 630 | 7489710 | 682 | LIEALRHCLDTYRKYGES-WKSLQARGMSQDLSWDHAAELYEDVLVKAKYQW | 732 |
| 631 | 8953571 | 748 | LIEALGHCLRTYRDYKES-WRGLQERGMSQDFSWEHAAKLYEDVLVKAKYQW | 798 |
| 632 | 8953571 | 749 | LIEALGHCLRTYRDYKES-WRGLQERGMSQDFSWEHAAKLYEDVLLKAKYQW | 799 |
| 633 | 5825480 | 749 | LIEALGHCLRTYRDYKES-WRGLQERGMSQDFSWEHAAKLYEDVLLKAKYQW | 799 |
| 634 | 7529653 | 749 | LIEALGHCLRTYRDFKES-WRALQERGMSQDFSWEHAAKLYEDVLVKAKYQW | 799 |
| 635 | 16265834 | 760 | LIEALGHCLETYRKYKES-WRGLQVRGMSQDLSWDHAAELYEEVLVKAKYQW | 810 |
| 636 | 7489695 | 441 | LIEALGHCLRTYRDFKES-WRALQERGMSQDFSWEHAAKLYEDVLVKAKYQW | 491 |
| 637 | 15384987 | 587 | MIDALGHCLNTYRNYKES-WRGLQARGMAQDLSWDHAAELYEDVLVKAKYQW | 637 |
| 638 | 15028467 | 644 | MIDALGHCLNTYRNYKES-WRGLQARGMAQDLSWDHAAELYEDVLVKAKYQW | 694 |
| 639 | 7489711 | 648 | MIDALSHCLTTYRNYKES-WRACRARGMAEDLSWDHAAVLYEDVLVKAKYQW | 698 |
| 640 | 2833384 | 702 | LMAALWNCLLTYKDYKKS-WEGIQERGMSQDLSWDNAAQQYEEVLVAAKYQW | 752 |
| 641 | 2129898 | 702 | LMAALWNCLLTYKDYKKS-WEGIQERGMSQDLSWDNAAQQYEEVLVAAKYQW | 752 |
| 642 | 6467503 | 701 | LIHALGNCLLTYREYKKS-WEGLQRRGMTPNLSWDHAAEKYEETLVAAKYQW | 751 |
| 643 | 15232051 | 742 | LIHALGNCLLTYREYKES-WEGLQRRGMTQDLSWDNAAEKYEEVLVAAKYHW | 792 |
| 644 | 3192881 | 580 | LIDALGNCLLTYRQYKQS-WEGLQRRGMMQDLSWDHAAEKYEEVLVAAKYQW | 630 |
| 645 | 2833390 | 738 | LIPRIRNCLLTYREYKKS-WEGIQTRCMTQDLSWDNAAQNYEEVLIAAKYQW | 788 |
| 646 | 14495348 | 699 | LIDALGHCLNTYRNYRTS-WEGLQKRGMMQDLSWDNAAKLYEEVLLAAKYQW | 749 |
| 647 | 8708896 | 526 | LRESINNALYTYRQFRDS-FRGIQRRGMEQDLTWDNAASIYEEVLVAAKYQW | 576 |
| 648 | 2833387 | 447 | MLWALRTAMSTFRENKPS-WEGLMKRGMTKDHTWDHA | 482 |
| 649 | 15237934 | 598 | MVSALRLAAATYREYKQS-WEGLMRRGMTRNYSWENAAVQYEQV-----FQW | 643 |
| 650 | 5880466 | 593 | MLWALRTAMSTFREHKPS-WEGLMKRGMTKDHTWDHAAEQYEQI | 635 |
| 651 | 6690399 | 521 | MVSALRLAAATYREYKQS-WEGLMRRGMTRNYSWENAAVQYEQV-----FQW | 566 |
| 652 | 9369334 | 593 | MLWALRTAMSTFREHKPS-WEGLMKRGMTKDHTWDHAAEQYEQI | 635 |
| 653 | 6103327 | 593 | MLWALRTAMSTFREHKPS-WEGLMKRGMTKDHTWDHAAEQYEQI | 635 |
| 654 | 7188796 | 589 | MLWALRTAISTFREHKPS-WEGLMKRGMTKDHTWDHAAEQYEQI | 631 |
| 655 | 2829792 | 587 | LLDTLKLAIGTYTEHKSS-WEGLMRRGMGRDYSWENAAIQYEQVFTWA | 633 |
| 656 | 7484400 | 272 | LRESINNALYTYRQFRDS-FRGIQRRGMEQDLTWDNAASIYEEVLVAAKYQW | 322 |
| 657 | 12019656 | 593 | MFVDIANC | 600 |
| 658 | 7489712 | 586 | MFVDIANC | 593 |
| 659 | 6136121 | 539 | IATTVERALAAYGSV--A-YKEMIQNCMAQDLSWKGPAKNWEKMLL | 581 |
| 660 | 5441242 | 537 | IATTVRRALGTYGTV--A-MEKIIQNCMAQDFSWKGPAKQWEKVL | 578 |
| 661 | 2833388 | 539 | IVKTVARALGTYAT--AA-LREMILNCMAQDLSWKGPARMWEKMLL | 581 |
| 662 | 3832512 | 538 | LATTVNRALKTYGT--QA-LKEMILNCMAQDFSWKGPAKQWEQALL | 580 |
| 663 | 15223331 | 538 | VIATAKAVTRAVAVYGTSAMQEMVKNCMDQDFSWKGPARLWEKVLL | 583 |
| 664 | 2833383 | 534 | LAATVKRALKTYGT--QA-MKQIILNCMAQNFSWKKPAKIWEKALL | 576 |
| 665 | 15637079 | 539 | VITTVGRALAMYGTL--A-FTEMIKNCMSQELSWKGPAKNWETVLL | 581 |
| 666 | 297196 | 538 | IVTTVARALAVYGTL--A-FAEMIKNCMSEELSWKEPAKKWETLLL | 580 |
| 667 | 228210 | 538 | IVTTVARALAVYGTL--A-FAEMIKNCMSEELSWKEPAKKWETLLL | 580 |
| 668 | 602594 | 538 | IVTTVARALAVYGTL--A-FAEMIKNCMSEELSWKEPAKKWETLLL | 580 |
| 669 | 2833381 | 539 | VITTVGRALAIYGTL--A-FTEMIKNCMSQELSWKGPAKNWETVLL | 581 |
| 670 | 12003285 | 535 | IVKTVGRALEVYGT--PA-FREMINNCMSLDLSWKGPAKNWETVLL | 577 |
| 671 | 15626365 | 541 | VDAIPKTVTKALGVYGTSAFAEMIKNCMAQELSWKGPAKYWEEVLL | 586 |
| 672 | 6624281 | 535 | VVTTLKRAVKVVGT--PA-YHEMVKNCMIQDLSWKGPAKNWEDVLLE | 578 |
| 673 | 6624287 | 535 | VVTTLKRAVKVVGT--PA-YHGMVKNCMIQDLSWKGPAKNWEDVLLE | 578 |
| 674 | 4760584 | 535 | VVTTLKRAVKVVGT--PA-YHEMVKNCMIQDLSWKGPAKNWEDVLLE | 578 |
| 675 | 18139611 | 539 | VASTVKRALKQYNT--PA-FQEMVQNCMAQDLSWKGPAKKWEEVLL | 581 |
| 676 | 17736918 | 465 | VVTTLKRAVKVVGT--PA-YHEMVKNCMIQDLSWKGPAKNWEDVLLE | 508 |

TABLE XXIV
Identities of the Accession Numbers used in Tables. XIX-XXIV.

| Accession | Brief Description of sequences | score | |
|---|---|---|---|
| Id. | producing significant alignments | (bits) | E-value |
| gi\|7489710\|pir\| \|T01208 | ADPglucose--starch glucosyltransfera . . . | 1389 | 0.0 |
| gi\|8953573\|emb\|CAB96627.1 (AJ269504) | starch synthase IIa-3 . . . | 976 | 0.0 |
| gi\|8953571\|emb\|CAB96626.1 (AJ269503) | starch synthase IIa-2 . . . | 976 | 0.0 |
| gi\|5825480\|gb\|AAD53263.1\|AF155217_1 (AF155217) | starch synth . . . | 975 | 0.0 |
| gi\|7529653\|emb\|CAB86618.1\| (AJ269502) | starch synthase IIa-1 . . . | 970 | 0.0 |
| gi\|16265834\|gb\|AAL16661.1\|AF419099_1 (AF419099) | putative so . . . | 968 | 0.0 |
| gi\|7489695\|pir\| \|T06798 | probable starch synthase (EC 2.4.1.- . . . | 910 | 0.0 |
| gi\|15384987\|emb\|CAC59826.1\| (AJ308110) | soluble starch synth . . . | 902 | 0.0 |
| gi\|15028467\|gb\|AAK81729.1\|AF395537_1 (AF395537) | soluble sta . . . | 892 | 0.0 |
| gi\|7489711\|pir\| \|T01209 | ADPglucose--starch glucosyltransfera . . . | 863 | 0.0 |
| gi\|2833384\|sp\|Q43093\|UGS3 PEA | Glycogen [starch] synthase, c . . . | 836 | 0.0 |
| gi\|2129898\|pir\| \|S61505 | UDPglucose--starch glucosyltransfera . . . | 835 | 0.0 |
| gi\|6467503\|gb\|AAF13168.1\|AF173900_1 (AF173900) | granule boun . . . | 832 | 0.0 |
| gi\|115232051\|ref\|(NP 186767.1\| (NM_110984) | putative glycogen . . . | 826 | 0.0 |
| gi\|3192881\|gb\|AAC19119.1\| (AF068834) | starch synthase [Ipomo . . . | 813 | 0.0 |
| gi\|2833390\|sp\|Q43847\|UGS3 SOLTU | Glycogen [starch] synthase, . . . | 788 | 0.0 |
| gi\|14495348\|gb\|AAK64284.1\|AF383878_1 (AF383878) | soluble sta . . . | 786 | 0.0 |
| gi\|8708896\|gb\|AAC17970.21 (AF026421) | soluble starch synthas . . . | 692 | 0.0 |
| gi\|2833387\|sp\|Q43654\|UGS2 WHEAT | Soluble glycogen [starch] s . . . | 448 | e−125 |
| gi\|15237934\|ref\|NP 197818.1\| (NM_122336) | soluble starch syn . . . | 446 | e−124 |
| gi\|5880466\|gb\|AAd54661.1\| (AF091803) | starch synthase I [Tri . . . | 444 | e−123 |
| gi\|6690399\|gb\|AAF24126.1\|AF121673_1 (AF121673) | soluble star . . . | 444 | e−123 |
| gi\|9369334\|emb\|CAB99209.1\| (AJ292521) | starch synthase I-1 [ . . . | 444 | e−123 |
| gi\|6103327\|gb\|AAF03557.1\| (AF091802) | starch synthase I [Aeg . . . | 442 | e−123 |
| gi\|7188796\|gb\|AAF37876.1\|AF234163_1 (AF234163) | starch synth . . . | 441 | e−122 |
| gi\|2829792\|sp\|P93568\|UGS2 SOLTU | Soluble glycogen [starch] s . . . | 440 | e−122 |
| gi\|7484400\|pir\| \|T07924 | probable starch synthase (EC 2.4.1.- . . . | 428 | e−119 |
| gi\|12019656\|gb\|AAd45815.2\| (AF168786) | soluble starch syntha . . . | 412 | e−114 |
| gi\|7489712\|pir\| \|T01414 | AdPglucose--starch glucosyltransfera . . . | 407 | e−112 |
| gi\|2833377\|sp\|Q40739\|UGS2 ORYSA | Soluble glycogen [starch] s . . . | 391 | e−107 |
| gi\|1549232\|dbj\|BAA07396.1\| (D38221) | SSS1 [Oryza sativa] >gi . . . | 390 | e−107 |
| gi\|5295947\|dbj\|BAA81848.1\| (AB026295) | ESTs AU075322 (C11109) . . . | 390 | e−107 |
| gi\|6136121\|sp\|082627\|UGST ANTMA | Granule-bound glycogen [sta . . . | 350 | 3e−95 |
| gi\|5441242\|dbj\|BAA82346.1\| (AB029546) | granule-bound starch . . . | 344 | 2e−93 |
| gi\|2833388\|sp\|Q43784\|UGST MANES | Granule-bound glycogen [sta . . . | 342 | 9e−93 |
| gi\|3832512\|gb\|AAC70779.1\| (AF097922) | granule-bound glycogen . . . | 338 | 9e−92 |
| gi\|15223331\|ref\|Np_174566.1\| (NM_103023) | starch synthase, p . . . | 337 | 4e−91 |
| gi\|2833383\|sp\|Q43092\|UGST PEA | Granule-bound glycogen [starc . . . | 334 | 2e−90 |
| gi\|15637079\|dbj\|BA968126.1\| (AB071604) | granule-bound starch . . . | 333 | 5e−90 |
| gi\|2671961\|sp\|Q00775\|UST SOLTU | Granule-bound glycogen [star . . . | 332 | 1e−89 |
| gi\|228210\|prf\| \|11718316A | granule-bound starch synthase [Sola . . . | 330 | 2e−89 |
| gi\|602594\|emb\|CAA58220.1\| (X83220) | starch (bacterial glycog . . . | 330 | 2e−89 |
| gi\|2833381\|sp\|Q42857\|UGST IPOBA | Granule-bound glycogen [sta . . . | 327 | 2e−88 |
| gi\|12003285\|9b\|AAG43519.1\|AP210699_1 (AF210699) | granule-bou . . . | 327 | 2e−88 |
| gi\|15626365\|emb\|CAC69955.1\| (AJ345045) | granule-bound starch . . . | 327 | 3e−88 |
| gi\|6624281\|dbj\|BAA88509.1\| (AB029061) | starch synthase (GBSS . . . | 320 | 4e−86 |
| gi\|6624287\|dbj\|BAA88512.1\| (A3029064) | starch synthase (GBSS . . . | 319 | 7e−86 |
| gi\|4760584\|dbj\|BAA77352.1\| (AB019624) | starch synthase (GBSS . . . | 318 | 1e−85 |
| gi\|18139611\|gb\|AAL58572.1\| (AY069940) | granule binding starc . . . | 318 | 1e−85 |
| gi\|17736918\|gb\|AA41028.1\| (AF2S0137) | mutant granule bound . . . | 318 | 1e−85 |
| gi\|6318538\|gb\|AAF06936.1\|AF110373_1 (AF110373) | granule-boun . . . | 318 | 2e−85 |
| gi\|6624285\|dbj\|BAA88511.1\| (AB029063) | starch synthase (GBSS . . . | 317 | 4e−85 |
| gi\|6318540\|gb\|AAF06937.1\|AF110374_1 (AF110374) | granule-boun . . . | 316 | 5e−85 |
| gi\|4760582\|dbj\|BAA77351.1\| (AB019623) | starch synthase (GBSS . . . | 316 | 5e−85 |
| gi\|11037536\|gb\|AAG27624.1\|AF286320_1 (AF286320) | granule bou . . . | 316 | 6e−85 |
| gi\|136765\|sp\|P27736\|UGST WHEAT | Granule-bound glycogen [star . . . | 314 | 2e−84 |
| gi\|4760580\|dbj\|BAA77350.1\| (AB019622) | starch synthase (GESS . . . | 313 | 4e−84 |
| gi\|136755\|sp\|P09842\|UGST HORVU | Granule-bound glycogen [star . . . | 313 | 4e−84 |
| gi\|18652407\|gb\|AAL77109.1\|AF474373_6 | (AF474373) granule-bon . . . | 313 | 5e−84 |
| gi\|2833385\|sp\|Q43134\|UGST SORBI | Granule-bound glycogen [sta . . . | 313 | 5e−84 |
| gi\|6492245\|gb\|AAF14233.1\|AF109395_1 (AF109395) | granule-boun . . . | 312 | 8e−84 |
| gi\|6624283\|dbj\|BAA88510.11 (AB029062) | starch synthase (GBSS . . . | 311 | 1e−83 |
| gi\|4588609\|gb\|AAD26156.1\|AF113844_1 (AF113844) | granule-boun . . . | 311 | 2e−83 |
| gi\|16716335\|gb\|AAC17969.3\| (AF026420) | granule-bound starch . . . | 309 | 7e−83 |
| gi\|82478\|pir\| \|JQ0703 | UDPglucose--starch glucosyltransferasa . . . | 308 | 9e−83 |
| gi\|2833382\|sp\|Q42968\|UGST ORYGL | Granule-bound glycogen [sta . . . | 307 | 2e−82 |
| gi\|297424\|emb\|CAA46294.1\|(X65183) | glycogen (starch) syntha . . . | 306 | 5e−82 |
| gi\|136757\|sp\|P047l3 UGST MAIZE | Granule-bound glycogen [star . . . | 306 | 5e−82 |
| gi\|7798551\|gb\|AAC61675.2\| (AF031162) | granule-bound starch s . . . | 306 | 7e−82 |
| gi\|136758\|sp\|P19395\|UGST ORYSA | Granule-bound glycogen [star . . . | 305 | 8e−82 |
| gi\|297422\|emb\|CAA45472.1\| (X64108) | starch granule-hound sta . . . | 301 | 2e−80 |
| gi\|4588607\|gb\|AAD26155.1\|AF113843_1 (AF113843) | granule-boun . . . | 293 | 4e−78 |
| gi\|15643657\|ref\|NP_228703.1\| (NC_000853) | glycogen synthase . . . | 284 | 2e−75 |
| gi\|15900991\|ref\|NP_345595.1\| (NC_003028) | glycogen synthase . . . | 283 | 4e−75 |
| gi\|15903076\|ref\|NP_358626.1\| (NC_003098) | Glycogen synthase . . . | 283 | 5e−75 |
| gi\|15672681\|ref\|NP_266855.1\| (NC_002662) | glycogen synthase . . . | 273 | 4e−72 |
| gi\|17366711\|sp\|Q9CEM9\|GLGA LACLA | Glycogen synthase (Starch . . . | 272 | 8e−72 |
| gi\|18309046\|ref\|NP_560980.1\| (NC_003366) | glycogen synthase . . . | 264 | 2e−69 |
| gi\|16080147\|ref\|NP_390973.1\| (NC_000964) | starch (bacterial . . . | 263 | 6e−69 |
| gi\|15895507\|ref\|NP_348856.1\| (NC_003030) | Glycogen synthase, . . . | 261 | 2e−68 |
| gi\|2811062\|sp\|008328\|GLGA BACST | Glycogen synthase (Starch [ . . . | 257 | 2e−67 |
| gi\|17229371\|ref\|NP_485919.1\| (NC_003272) | glycogen synthase . . . | 251 | 2e−65 |
| gi\|15613648\|ref\|NP_241951.1\| (NC_002570) | starch (bacterial . . . | 248 | 1e−64 |
| gi\|1641730\|ref\|NP_231362.1\| (NC_002505) | glycogen synthase . . . | 234 | 2e−60 |
| gi\|16766821\|ref\|NP_462436.1\| (NC_003197) | glycogen synthase . . . | 233 | 5e−60 |
| gi\|16331219\|ref\|NP_441947.1\| (NC_000911) | glycogen synthase . . . | 231 | 2e−59 |
| gi\|15620537\|gb\|AAL03921.1\|U30252_9 (U30252) | GlgA [Synechoco . . . | 231 | 3e−59 |
| gi\|16762766\|ref\|NP_458383.1\| (NC_003198) | glycogen synthase . . . | 231 | 3e−59 |
| gi\|17938870\|ref\|NP_535658.1\| (NC_003306) | glycogen synthase . . . | 222 | 1e−56 |
| gi\|16119514\|ref\|NP_396220.1\| (NC_003064) | AGR_pAT_410p [Agro . . . | 221 | 1e−56 |
| gi\|16124067\|ref\|NP_407380.1\| (NC_003143) | glycogen synthase . . . | 219 | 6e−56 |
| gi\|6116748\|dbj\|BAA85761.1\| (AB028026) | granule-bound starch . . . | 219 | 7e−56 |
| gi\|15803938\|\|ref NP_289974.1\| (NC_002655) | glycogen synthase . . . | 219 | 1e−55 |
| gi\|15966599\|ref\|NP_386952.1\| (NC_003047) | PROBABLE GLYCOGEN . . . | 217 | 4e−55 |
| gi\|15890897\|ref\|NP_356569.1\| (NC_003063) | AGR_L_1562p [Agrob . . . | 216 | 8e−55 |
| gi\|13476305\|ref\|NP_107875.1\| (NC_002678) | glycogen synthase . . . | 215 | 2e−54 |
| gi\|14279432\|gb\|AAK58595.1\|AF268959_2 (AF268969) | glycogen sy . . . | 214 | 4e−54 |
| gi\|17366749\|sp\|Q9EUT5\|GLGA_RHITR | Glycogen synthase (Starch . . . | 213 | 5e−54 |
| gi\|15717885\|gb\|AAK97773.1\| (AY044844) | starch synthase isofo . . . | 213 | 7e−54 |
| gi\|4582783\|emb\|CAB40375.1\| (AJ006752) | starch synthase, isof . . . | 213 | 7e−54 |
| gi\|16329217\|ref\|NP_439945.1\| (NC_000911) | glycogen (starch) . . . | 211 | 2e−53 |
| gi\|17227527\|ref\|NP_484075.1\| (NC_003272) | glycogen (starch) . . . | 211 | 3e−53 |
| gi\|9587293\|gb\|AAF89248.1\|AF285973_1 (A9285973) | granule-boun . . . | 209 | 8e−53 |
| gi\|9587321\|gb\|AAF89262.1\|AF285987_1 (AF285987) | granule-boun . . . | 209 | 1e−52 |
| gi\|8901183\|gb\|AAC17971.2\| (AF026422) | soluble starch synthas . . . | 208 | 1e−52 |
| gi\|15602409\|ref\|NP_245481.1\| (NC_002663) | GlgA [Pasteurella . . . | 208 | 2e−52 |
| gi\|9587317\|gb\|AAF89260.1\|AF285985_1 (AF285985) | granule-boun . . . | 207 | 5e−52 |
| gi\|9587301\|gb\|AAF89252.1\|AF285977_1 (AF285977) | granule-boun . . . | 206 | 8e−52 |
| gi\|15605118\|ref\|NP_213495.1\| (NC_000918) | glycogen synthase . . . | 205 | 2e−51 |
| gi\|146139\|gb\|AAA23870.1\| (J02616) | glycogen synthase (EC 2.4 . . . | 204 | 2e−51 |
| gi\|16265159\|ref\|NP_437951.1\| (NC_003078) | putative glycogen . . . | 204 | 2e−51 |
| gi\|9587329\|gb\|AAF89266.1\|AF28599_1 (AF285991) | granula-boun . . . | 204 | 3e−51 |
| gi\|9587352\|gb\|AAF89276.1\|AF286003_1 (AF286003) | granula-boun . . . | 203 | 7e−51 |
| gi\|9587311\|gb\|AAF89257.1\|AF285982_1 (AF285982) | granule-boun . . . | 199 | 1e−49 |
| gi\|9587305\|gb\|AAF89254.1\|AF285979 1 (AF285979) | granule-boun . . . | 198 | 1e−49 |
| gi\|9587343\|gb\|AAF89273.1\|A9285998 1 (AF285998) | granule-boun . . . | 198 | 2e−49 |
| gi\|17548463\|ref\|NP_521803.1\| (NC_003296) | PROBABLE GLYCOGEN . . . | 198 | 2e−49 |
| gi\|18461221\|dbj\|BAb84418.1\| (AP003292) | putative starch synt . . . | 197 | 2e−49 |
| gi\|9587337\|gb\|AAF89270.1\|AF285995_1 (AF285995) | granule-boun . . . | 197 | 3e−49 |
| gi\|16273270\|ref\|NP_439511.1\| (NC_000907) | glycogen synthase . . . | 196 | 5e−49 |
| gi\|9587348\|gb\|AAF89274.1\| (AF286001) | granule-bound starch s . . . | 196 | 5e−49 |
| gi\|9587341\|gb\|AAF89272.1\|AF285997 1(AF285997) | granule-boun . . . | 196 | 6e−49 |
| gi\|9587323\|gb\|AAF89263.1\|AF285988 1(AF285988) | granule-boun . . . | 196 | 7e−49 |
| gi\|15236819\|ref\|NP_193558.1\| (NM 117934) | starch synthase-li . . . | 194 | 2e−48 |
| gi\|9587307\|gb\|AA989255.1\|AF285980_1 (AF285980) | granule-boun . . . | 194 | 2e−48 |
| gi\|9587327\|gb\|AAF89265.1\|AF285990_1 (AF285990) | granule-boun . . . | 194 | 3e−48 |
| gi\|9587319\|gb\|AAF89261.1\|AF285986_1 (AF285986) | granule-boun . . . | 194 | 4e−48 |
| gi\|15983795\|gb\|AAL10494.1\| (AY056803) | Atlg32900/F9L11_8 [Ar . . . | 193 | 5e−48 |
| gi\|9587313\|gb\|AAF89258.1\|AF285983_1 (AF285983) | granule-boun . . . | 193 | 7e−48 |
| gi\|9587297\|gb\|AAF89250.1\|AF285975_1 (AR285975) | granule-boun . . . | 193 | 7e−48 |
| gi\|9587309\|gb\|AAF89256.1\|AF285981_1 (AF285981) | granule-boun . . . | 192 | 8e−48 |
| gi\|9587299\|gb\|AAF89251.1\|AF285976_1 (AF285976) | granule-boun . . . | 192 | 8e−48 |
| gi\|9587333\|gb\|AAF89268.1\|AF285993_1 (AF285993) | granule-boun . . . | 192 | 9e−48 |
| gi\|9587325\|gb\|AAF89264.1\|AF285989_1 (AF285989) | granule-boun . . . | 191 | 2e−47 |
| gi\|5834407\|gb\|AAD53959.1\|AF181035_3 (AF181035) | glycogen syn . . . | 191 | 3e−47 |
| gi\|17367070\|sp\|Q9RNH6\|GLGA RHOSH | Glycogen synthase (Starch . . . | 191 | 3e−47 |
| gi\|15221083\|ref\|NP_172637.1\| (NM_101044) | putative glycogen . . . | 190 | 4e−47 |
| gi\|15597361\|ref\|NP_250855.1\| (NC_002516) | probable glycogen . . . | 189 | 1e−46 |
| gi\|9587331\|gb\|AAF89267.1\|AF285992_1 (AF285992) | granule-boun . . . | 189 | 1e−46 |
| gi\|7489826\|pir\| \|T01265 | starch synthase DULL1 - maize >gi\|30 . . . | 188 | 2e−46 |
| gi\|9587303\|gb\|AAF89253.1\|AF285978_1 (AF285978) | granule-boun . . . | 188 | 2e−46 |
| gi\|4582789\|emb\|CAB40374.1\| (AJ225088) | Starch synthase isofo . . . | 188 | 2e−46 |
| gi\|9587339\|gb\|AAF89271.1\|AF285996_1 (AP285996) | granula-boun . . . | 187 | 4e−46 |
| gi\|9587335\|gb\|AAF89269.1\|AF285994_1 (AF285994) | granule-boun . . . | 187 | 4e−46 |
| gi\|9587295\|gb\|AAF89249.1\|AF285974_1 (AF285974) | granule-boun . . . | 186 | 6e−46 |
| gi\|15805621\|ref\|NP_294317.1\| (NC_001263) | glycogen synthase . . . | 184 | 4e−45 |
| gi\|17367076\|sp\|Q9RWS1\|GLGA DEIRA | Glycogen synthase (Starch . . . | 183 | 6e−45 |
| gi\|7489274\|pir\| \|T07663 | soluble starch synthase (EC 2.4.1.-) . . . | 183 | 7e−45 |
| gi\|2833389\|sp\|Q43846\|UGS4 SOLTU | Soluble glycogen [starch]s . . . | 181 | 3e−44 |
| gi\|9502145\|gb\|AAF88000.1\| (AF258609) | starch synthase III [A . . . | 179 | 8e−44 |
| gi\|9502143\|gb\|AAF87999.1\|AF258608_1 (AF258608) | starch synth . . . | 177 | 2e−43 |
| gi\|7484399\|pir\| \|T07926 | probable starch synthase (EC 2.4.1.- . . . | 177 | 3e−43 |
| gi\|17366350\|sp\|P58395\|GLGA THECA | Glycogen synthase (Starch . . . | 176 | 5e−43 |
| gi\|17646328\|gb\|AAL40942.1 AF432915_1 (AF432915) | putative st . . . | 175 | 1e−42 |
| gi\|12278503\|gb\|AAG48992.1\| (AY011005) | granule-bound starch . . . | 170 | 5e−41 |
| gi\|3493007\|gb\|AAD02961.1\| (AF079241) | granule-bound starch s . . . | 170 | 6e−41 |
| gi\|12278507\|gb\|AAG48994.1\| (AY011007) | granule-bound starch . . . | 170 | 6e−41 |
| gi\|3493065\|gb\|AAD02990.1\| (AF079270) | granule-bound starch s . . . | 169 | 8e−41 |
| gi\|3493055\|gb\|AAD02985.1\| (AF079265) | granule-bound starch s . . . | 169 | 1e−40 |
| gi\|3493079\|gb\|AAD02997.1\| (AF079277) | granule-bound starch s . . . | 169 | 1e−40 |
| gi\|12278417\|gb\|AAG48949.1\| (AY010962) | granule-bound starch . . . | 169 | 1e−40 |
| gi\|3493057\|gb\|AAD02986.1\| (AF079266) | granule-bound starch s . . . | 169 | 1e−40 |
| gi\|3493061\|gb\|AAD02988.1\| (AF079268) | granule-bound starch s . . . | 169 | 1e−40 |
| gi\|12278505\|gb\|AAG48993.1\| (AY011006) | granule-bound starch . . . | 169 | 1e−40 |
| gi\|3493059\|gb\|AAD02987.1\| (AF079267) | granule-bound starch s . . . | 169 | 1e−40 |
| gi\|3493107\|gb\|AAD03011.1\| (AF079291) | granule-bound starch s . . . | 168 | 2e−40 |
| gi\|3493049\|gb\|AAD02982.1\| (AF079262) | granule-bound starch s . . . | 168 | 2e−40 |
| gi\|12278487\|gb\|AAG48984.1\| (AY010997) | granule-bound starch . . . | 168 | 2e−40 |
| gi\|3493089\|gb\|AAD03002.1\| (AF079282) | granule-bound starch . . . | 168 | 2e−40 |
| gi\|12278473\|gb\|AAG48977.1\| (AY010990) | granule-bound starch . . . | 168 | 2e−40 |
| gi\|3493051\|gb\|AAD02983.1\| (AF079263) | granule-bound starch s . . . | 167 | 2e−40 |
| gi\|12278427\|gb\|AAG48954.1\| (AY010967) | granule-bound starch . . . | 167 | 3e−40 |
| gi\|12278497\|gb\|AAG48989.1\| (AY011002) | granule-bound starch . . . | 167 | 3e−40 |
| gi\|3493005\|gb\|AAD02960.1\| (AF079240) | granule-bound starch s . . . | 167 | 3e−40 |
| gi\|12278463\|gb\|AAG48972.1\| (AY010985) | granule-bound starch . . . | 167 | 3e−40 |
| gi\|12278509\|gb\|AAG48995.1\| (AYO11008) | granule-bound starch . . . | 167 | 3e−40 |
| gi\|3493093\|gb\|AAD03004.1\| (AF079284) | granule-bound starch s . . . | 167 | 3e−40 |
| gi\|3493073\|gb\|AAD02994.1\| (AF079274) | granule-bound starch s . . . | 167 | 4e−40 |
| gi\|12278471\|gb\|AAG48976.1\| (AY010989) | granule-bound starch . . . | 167 | 4e−40 |
| gi\|3493019\|gb\|AAD02967.1\| (AF079247) | granule-bound starch s . . . | 167 | 5e−40 |
| gi\|12278453\|gb\|AAG48967.1\| (AY010980) | granule-bound starch . . . | 167 | 5e−40 |
| gi\|12278491\|gb\|AAG48986.1\| (AY010999) | granule-bound starch . . . | 166 | 6e−40 |
| gi\|12278451\|gb\|AAG48966.1\| (AY010979) | granule-bound starch . . . | 166 | 6e−40 |
| gi\|3493077\|gb\|AAD02996.1\| (AF079276) | granule-bound starch s . . . | 166 | 7e−40 |
| gi\|3493067\|gb\|AAD02991.1\| (AF079271) | granule-bound starch s . . . | 166 | 7e−40 |
| gi\|3493097\|gb\|AAD03006.1\| (AF079286) | granule-bound starch s . . . | 166 | 7e−40 |
| gi\|12278514\|gb\|AAG48997.1\| (AY011011) | granule-bound starch . . . | 166 | 7e−40 |
| gi\|3493121\|gb\|AAD03018.1\| (AF079298) | granule-bound starch s . . . | 166 | 8e−40 |
| gi\|3493091\|gb\|AAD03003.1\| (AF079283) | granule-bound starch s . . . | 166 | 8e−40 |
| gi\|12278481\|gb\|AAG48981.1\| (AY010994) | granule-bound starch . . . | 166 | 8e−40 |
| gi\|3493083\|gb\|AAD02999.1\| (AF079279) | granule-bound starch s . . . | 166 | 8e−40 |
| gi\|3493111\|gb\|AAD03013.1\| (AF079293) | granule-bound starch s . . . | 166 | 8e−40 |
| gi\|12278457\|gb\|AAG48969.1\| (AY010982) | granule-bound starch . . . | 166 | 9e−40 |
| gi\|13774482\|gb\|AAK38880.1\| (AF353518) | granule-bound starch . . . | 166 | 9e−40 |
| gi\|12278479\|gb\|AAG48980.1\| (AY010993) | granule-bound starch . . . | 166 | 9e−40 |
| gi\|3493095\|gb\|AAD03005.1\| (AF079285) | granule-bound starch s . . . | 166 | 1e−39 |
| gi\|3493081\|gb\|AAD02998.1\| (AF079278) | granule-bound starch s . . . | 166 | 1e−39 |
| gi\|112278429\|gb\|AA048955.1\| (AY010968) | granule-bound starch . . . | 166 | 1e−39 |
| gi\|3493087\|gb\|AAD03001.1\| (AF079281) | granule-bound starch s . . . | 166 | 1e−39 |
| gi\|12278485\|gb\|AAG48983.1\| (AY010996) | granule-bound starch . . . | 166 | 1e−39 |
| gi\|3493025\|gb\|AAD02970.1\| (AF079250) | granule-bound starch s . . . | 165 | 1e−39 |
| gi\|13774486\|gb\|AAK38882.1\| (AF353520) | granule-bound starch . . . | 165 | 1e−39 |
| gi\|3493117\|gb\|AAD03016.1\| (AF079296) | granule-bound starch s . . . | 165 | 1e−39 |
| gi\|12278425\|gb\|AAG48953.1\| (AY010966) | granule-bound starch . . . | 165 | 2e−39 |
| gi\|3493031\|gb\|AAD02973.1\| (AF079253) | granule-bound starch s . . . | 164 | 2e−39 |
| gi\|12278499\|gb\|AAG48990.1\| (AY011003) | granule-bound starch . . . | 164 | 3e−39 |
| gi\|12278443\|gb\|AAG48962.1\| (AY010975) | granule-bound starch . . . | 164 | 3e−39 |
| gi\|12278511\|gb\|AAG48996.1\| (AY011009) | granule-bound starch . . . | 164 | 3e−39 |
| gi\|12278495\|gb\|AAG48988.1\| (AY011001) | granule-bound starch . . . | 164 | 3e−39 |
| gi\|3493099\|gb\|AAD03007.1\| (AF079287) | granule-bound starch s . . . | 164 | 3e−39 |
| gi\|12278411\|gb\|AAG48946.1\| (AY010959) | granule-bound starch . . . | 164 | 3e−39 |
| gi\|13377473\|gb\|AAK20725.1\| (AF318769) | granule-bound starch . . . | 164 | 3e−39 |
| gi\|12278501\|gb\|AAG48991.1\| (AY011004) | granule-bound starch . . . | 164 | 3e−39 |
| gi\|12278449\|gb\|AAG48965.1\| (AY010978) | granule-bound starch . . . | 164 | 4e−39 |
| gi\|3493103\|gb\|AAD03009.1\| (AF079289) | granule-bound starch s . . . | 164 | 4e−39 |
| gi\|12278477\|gb\|AAG48979.1\| (AY010992) | granule-bound starch . . . | 164 | 4e−39 |
| gi\|12278423\|gb\|AAG48952.1\| (AY010965) | granule-bound starch . . . | 164 | 4e−39 |
| gi\|122784211\|gb\|AAG48951.1\| (AY010964) | granule-bound starch . . . | 164 | 4e−39 |
| gi\|12278489\|gb\|AAG48985.1\| (AY010998) | granule-bound starch . . . | 164 | 4e−39 |
| gi\|13774484\|gb\|AAK38881.1\| (AF353519) | granule-bound starch . . . | 164 | 4e−39 |
| gi\|3493023\|gb\|AAD02969.1\| (AF079249) | granule-bound starch s . . . | 164 | 4e−39 |
| gi\|13377475\|gb\|AAK20726.1\| (AF318770) | granule-bound starch . . . | 164 | 4e−39 |
| gi\|12278435\|gb\|AAG48985.1\| (AY010971) | granule-bound starch . . . | 164 | 4e−39 |
| gi\|12278469\|gb\|AAG48975.1\| (AY010988) | granule-bound starch . . . | 163 | 5e−39 |
| gi\|3493101\|gb\|AAD03008.1\| (AF079288) | granule-bound starch s . . . | 163 | 5e−39 |
| gi\|3493113\|gb\|AAD03014.1\| (AF079294) | granule-bound starch s . . . | 163 | 5e−39 |
| gi\|12278419\|gb\|AAg48950.1\| (AY010963) | granule-bound starch . . . | 163 | 6e−39 |
| gi\|3493071\|gb\|AAD02993.1\| (AF079273) | granule-bound starch s . . . | 163 | 6e−39 |
| gi\|3493053\|gb\|AAD02984.1\| (AF079264) | granule-bound starch s . . . | 163 | 6e−39 |
| gi\|3493075\|gb\|AAD02995.1\| (AF079275) | granule-bound starch s . . . | 163 | 7e−39 |
| gi\|12278475\|gb\|AAG48978.1\| (AY010991) | granule-bound starch . . . | 162 | 8e−39 |
| gi\|3493041\|gb\|AAG42978.1\| (AF079258) | granule-bound starch s . . . | 162 | 9e−39 |
| gi\|12278433\|gb\|AAG48957.1\| (AY010970) | granule-bound starch . . . | 162 | 9e−39 |
| gi\|3493037\|gb\|AAD02976.1\| (AF079256) | granule-bound starch s . . . | 162 | 1e−38 |
| gi\|3493017\|gb\|AAD02966.1\| (AF079246) | granule-bound starch s . . . | 162 | 1e−38 |
| gi\|3493015\|gb\|AAD02965.1\| (AF079245) | granule-bound starch s . . . | 162 | 1e−38 |
| gi\|12278437\|gb\|AAG48959.1\| (AY010972) | granule-bound starch . . . | 162 | 1e−38 |
| gi\|3493021\|gb\|AAD02968.1\| (AF079248) | granule-bound starch s . . . | 162 | 1e−38 |
| gi\|3493069\|gb\|AAD02992.1\| (AF079272) | granule-hound starch s . . . | 162 | 1e−38 |
| gi\|3493027\|gb\|AAD02971.1\| (AF079251) | granule-bound starch s . . . | 161 | 2e−38 |
| gi\|3493013\|gb\|AAD02964.1\| (AF079244) | granule-bound starch s . . . | 161 | 2e−38 |
| gi\|3493011\|gb\|AAD02963.1\| (AF079243) | granule-bound starch s . . . | 161 | 2e−38 |
| gi\|12278493\|gb\|AAG48987.1\| (AY011000) | granule-bound starch . . . | 161 | 2e−38 |
| gi\|12278413\|gb\|AAG48947.1\| (AY010960) | granule-bound starch . . . | 161 | 2e−38 |
| gi\|3493085\|gb\|AAD03000.1\| (AF079280) | granule-bound starch s . . . | 161 | 3e−38 |
| gi\|3493009\|gb\|AAD02962.1\| (AF079242) | granule-bound starch s . . . | 161 | 3e−38 |
| gi\|3493035\|gb\|AAD02975.1\| (AF079255) | granule-bound starch s . . . | 160 | 4e−38 |
| gi\|3493063\|gb\|AAD02989.1\| (AF079269) | granule-bound starch s . . . | 160 | 4e−38 |
| gi\|12278431\|gb\|AAG48956.1\| (AY010969) | granule-bound starch . . . | 160 | 4e−38 |
| gi\|3492999\|gb\|AAD02957.1\| (AF079237) | granule-bound starch s . . . | 160 | 5e−38 |

SSIIb

```
Accession: AF019297
            NID: g2655030
                      Mol. wt. (calc) = 75458 Residues = 698
```

SEQ.ID. No. 677

| | |
|---|---|
| 1 | M P G A I S S S S S A F L L P V A S S S P R R R R G S V G A |
| 31 | A L R S Y G Y S G A E L R L H W A R R G P P Q D G A A S V R |
| 61 | A A A A P A G G E S E E A A K S S S S S Q A G A V Q G S T A |
| 91 | K A V D S A S P P N P L T S A P K Q S Q S A A M Q N G T S G |
| 121 | G S S A S T A A P V S G P K A D H P S A P V T K R E I D A S |
| 151 | A V K P E P A G D D A R P V E S I G I A E P V D A K A D A A |
| 181 | P A T D A A A S A P Y D R E D N E P G P L A G P N V M N V V |
| 211 | V V A S E C A P F C K T G G L C D V V G A L P K A L A R R G |
| 241 | H R V M V V I P R Y G E Y A E A R D L G V R R R Y K V A G Q |
| 271 | D S E V T Y F H S Y I D G V D F V F V E A P P F R H R H N N |
| 301 | I Y C G E R L D I L K R M I L F C K A A V E V P W Y A P C G |
| 331 | G T V Y G D G N L V F I A N D W H T A L L P V Y L K A Y Y R |
| 361 | D N G L M Q Y A R S V L V I H N I A H Q G R G P V D D F V N |
| 391 | F D L P E H Y I D H F K L Y D N I G G D H S N V F A A G L K |
| 421 | T A D R V V T V S N G Y M W E L K T S E G G W G L H D I I N |
| 451 | Q N D W K L Q G I V N G I D M S E W N P A V D V H L H S D D |
| 481 | Y T N Y T F E T L D T G K R Q C K A A L Q R Q L G L Q V R D |
| 511 | D V P L I G F I G R L D H Q K G V D I I A D A I H W I A G Q |
| 541 | D V Q L V M L G T G R A D L E D M L R R F E S E H S D K V R |
| 571 | A W V C F S V P L A H R I T A G A D I L L M P S R F E P C G |
| 601 | L N Q L Y A M A Y G T V P V V H A V G G L R D T V A P F D P |
| 631 | F N D T G L G W T F D R A E A N R M I D A L S H C L T T Y R |
| 661 | N Y K E S W R A C R A R G M A E D L S W D H A A V L Y E D V |
| 691 | L V K A K Y Q W |

TABLE XXV
Maize soluble starch synthase IIb (SSIIb)
Alignments with other similar proteins-Transit Peptide

| SEQ | Accession | a.a | | a.a. |
|---|---|---|---|---|
| Id.No. | Number | # | (start) Sequence ending | # |
| 678 | MAIZE SSIIb | 1 | MPGAISSSSSAFL-LXXXXXXXXXXXXXXXXALRSYGYSGAELRLHWARRGPPQDXXXXX | 59 |
| 679 | 7489711 | 1 | MPGAISSSSSAFL-LPVASSSPRRRRGSVGAALRSYGYSGAELRLHWARRGPPQDGAASV | 59 |
| 680 | 15028467 | 1 | MSGAIASSPAATLFLAGSSSSSPRRRRSRVSGVWWHLYGGTGLRLHWERRGLVRDGAVVC | 60 |
| Cont . . . d | | | | |
| 678 | Maize SSIIb | 61 | XXXXXXXXXXXXXXXXXXXXXXXXXXXXXXXKAVDSASPPNPLTSAPKQSQSA-AMQNXX | 118 |
| 679 | 7489711 | 60 | RAAAAPAGGESEEAAKSSSSSQAGAVQGSTAKAVDSASPPNPLTSAPKQSQSA-AMQNGT | 118 |
| 680 | 15028467 | 61 | SASAAGGEDGVAKAKTKSAGSSKAVAVQGSTAKADHVEDS---VSSPKYVKPAVAKQNGE | 117 |
| 681 | 15384987 | 45 | SSPKSVKPAVAKQNGE | 60 |
| Cont . . . d | | | | |
| 678 | Maize SSIIb | 119 | XXXXXXXXXXPVSGPKADHPSAPVTKREID--ASAVKPEPAGDDARPVESIGIXXXXXXX | 176 |
| 679 | 7489711 | 119 | SGGSSASTAAPVSGPKADHPSAPVTKREID--ASAVKPEPAGDDARPVESIGIAEPVDAK | 176 |
| 680 | 15028467 | 118 | VVSRATKSDAPVSKPKVD-PSVPASKAEADGNAQAVESKAALDKK---EDVGVAEPLEAK | 173 |
| 681 | 15384987 | 61 | VVSRATKSDAPVPKPKVD-PSVPASKAEADGNAQAVESKAALDKK---EDVGVAEPLEAK | 116 |

TABLE XXVI
Maize soluble starch synthase IIb (SSIIb)
“GLASS” Domain Alignments with other similar proteins

| SEQ | Accession | a.a | | a.a. |
|---|---|---|---|---|
| Id.No. | Number | # | (start Sequence end | # |
| 682 | MaizeSSIIb | 177 | XXXXXXXXXXXXXXYDREDNEPGPLAGPNV-----MNVVVVASECAPFCKTGGLGDVVGA | 231 |
| 683 | 7489711 | 177 | ADAAPATDAAASAPYDREDNEPGPLAGPNV-----MNVVVVASECAPFCKTGGLGDVVGA | 231 |
| 684 | 15028467 | 174 | ADAGGDAGAVSSAD-DSENKESGPLAGPNV-----MNVIVVASECSPFCKTGGLGDVVGA | 227 |
| 685 | 15384987 | 117 | ADAGGDAGAVSSAD-DSENKESGPLAGPNV-----MNVIVVASECSPFCKTGGLGDVVGA | 170 |
| 686 | 7489710 | 229 | DNDSGPLAGENV-----MNVIVVAAECSPWCKTGGLGDVVGA | 265 |
| 687 | 16265834 | 306 | QDDDSGPLAGENV-----MNVIVVAAECSPWCKTGGLGDVAGA | 343 |
| 688 | 15232051 | 287 | KDEEKPPPLAGANV-----MNVILVAAECAPFSKTGGLGDVAGA | 325 |
| 689 | 7489695 | 1 | NVVVVAAECSPWCKTGGLGDVAGA | 24 |
| 690 | 6467503 | 247 | EDMKPPPLAGDNV-----MNVILVAAECAPWSKTGGLGDVAGS | 284 |
| 691 | 2833384 | 245 | FESGGEKPPPLAGTNV-----MNIILVSAECAPWSKTGGLGDVAGS | 285 |
| 692 | 2129898 | 245 | FESGGEKPPPLAGTNV-----MNIILVSAECAPWSKTGGLGDVAGS | 285 |
| 693 | 3192881 | 126 | EDLKPPPLAGTNV-----MNVILVCAECAPWSKTGGLGDVAGA | 163 |
| 694 | 8953571 | 294 | QNHDSGPLAGENV-----MNVVVVAAECSPWCKTGGLGDVAGA | 331 |
| 695 | 7529653 | 295 | QNHDSGPLAGENV-----MNVVVVAAECSPWCKTGGLGDVAGA | 332 |
| 696 | 8953571 | 295 | QNHDSGPLAGENV-----MNVVVVAAECSPWCKTGGLGDVAGA | 332 |
| 697 | 5825480 | 295 | QNHDSGPLAGENV-----MNVVVVAAECSPWCKTGGLGDVAGA | 332 |
| 698 | 14495348 | 247 | DDPSASASVDL-----INIILVAAECAPWSKTGGLGDVAGA | 282 |
| 699 | 2833390 | 284 | DEKPPPLAGTNV-----MNIILVASECAPWSKTGGLGDVAGA | 320 |
| 700 | 8708896 | 75 | KPPPLAGPNV-----MNVVMVGAECAPWSKTGGLGDVMAA | 109 |
| 701 | 5880466 | 140 | SIVFVTGEAAPYAKSGGLGDVCGS | 163 |
| 702 | 9369334 | 140 | SIVFVTGEAAPYAKSGGLGDVCGS | 163 |
| 703 | 15237934 | 141 | V-----NNLVFVTSEAAPYSKTGGLGDVCGS | 166 |
| 704 | 7188796 | 136 | SIVFVTGEAAPYAKSGGLGDVCGS | 159 |
| 705 | 6103327 | 140 | SIVFVTGEAAPYAKSGGLGDVCGS | 163 |
| 706 | 9369336 | 140 | SIVFVTGEAAPYAKSGGLGDVCGS | 163 |
| 707 | 6690399 | 64 | V-----NNLVFVTSEAAPYSKTGGLGDVCGS | 89 |
| 708 | 2829792 | 118 | DTEEMEETPIK--LT-----FNIIFVTAEAAPYSKTGGLGDVCGS | 155 |
| 709 | 2833387 | 1 | EAAPYAKSGGLGDVCGS | 17 |
| 710 | 12019656 | 140 | SIVFVTGEASPYAKSGGLGDVCGS | 163 |
| 711 | 7489712 | 133 | SIVFVTGEASPYAKSGGLGDVCGS | 156 |
| 712 | 5295947 | 134 | SVVFVTGEASPYAKSGGLGDVCGS | 157 |
| 713 | 1549232 | 134 | SVVFVTGEASPYAKSGGLGDVCGS | 157 |
| 714 | 2833377 | 134 | SVVFVTGEASPYAKSGGLGDVCGS | 157 |
| 715 | 6136121 | 82 | -----MNLVFVLAEVGPWSKTGGLGDVVGG | 106 |
| 716 | 228210 | 81 | -----MNLIFVGTEVGPWSKTGGLGDVLGG | 105 |
| 717 | 297196 | 81 | -----MNLIFVGTEVGPWSKTGGLGDVLGG | 105 |
| 718 | 2833388 | 82 | -----MNLIFVGAEVGPWSKTGGLGDVLGG | 106 |
| 719 | 15637079 | 65 | ENEGGMAAGTIVCKQQGMNLVFVGCEVGPWCKTGGLGDVLGG | 106 |
| 720 | 602594 | 81 | -----MNLIFVGTEVGPWSKTGGLGDVLGG | 105 |
| 721 | 2833381 | 82 | -----MNLVFVGCEEGPWCKTGGLGDVLGG | 106 |
| 722 | 5441242 | 80 | -----MNLIFVGAEVAPWSKTGGLGDVLGG | 104 |
| 723 | 15223331 | 84 | -----MSVIFIGAEVGPWSKTGGLGDVLGG | 108 |
| 724 | 12003285 | 78 | -----MTLIFVSAECGPWSKTGGLGDVVGG | 102 |
| 725 | 3832512 | 79 | NG-----MNLVFVGAEVGPWSKTGGLGDVLGG | 105 |
| 726 | 15626365 | 87 | -----MNLIFVGTEVAPWSKTGGLGDVLGG | 111 |
| 727 | 2833383 | 77 | -----MSLVFVGAEVGPWSKTGGLGDVLGG | 101 |
| 728 | 6624281 | 73 | GSGG-----MNLVFVGAEMAPWSKTGGLGDVLGG | 101 |
| 729 | 17736918 | 3 | GSGG-----MNLVFVGAEMAPWSKTGGLGDVLGG | 31 |
| 730 | 4760584 | 73 | GSGG-----MNLVFVGAEMAPWSKTGGLGDVLGG | 101 |
| 731 | 6492245 | 63 | PAPIVCSTG-----MPIIFVATEVHPWCKTGGLGDVVGG | 96 |
| 682 | MaizeSSIIb | 232 | LPKALARRGHRVMVVIPRY-------GEYAEARDLG----VRRRYKVA--GQDSEVTYFH | 278 |
| 683 | 7489711 | 232 | LPKALARRGHRVMVVIPRY-------GEYAEARDLG----VRRRYKVA--GQDSEVTYFH | 278 |
| 684 | 15028467 | 228 | LPKALARRGHRVMVVIPRY-------GEYAEAKDLG----VRKRYRVA--GQDSEVSYFH | 274 |
| 685 | 15384987 | 171 | LPKALARRGHRVMVVIPRY-------GEYAEAKDLG----VRKRYRVA--GQDSEVSYFH | 217 |
| 686 | 7489710 | 266 | LPKALARRGHRVMVVVPRY-------GDYVEAFDMG----IRKYYKAA--GQDLEVNYFH | 312 |
| 687 | 16265834 | 344 | LPKALARRGHRVMVVVPRY-------GDYAEAQDVG----IRKYYKAA--GQDLEVKYFH | 390 |
| 688 | 15232051 | 326 | LPKSLARRGNRVMVVVPRY-------AEYAEAKDLG----VRKRYKVA--GQDMEVMYFH | 372 |
| 689 | 7489695 | 25 | LPKALAKRGHRVMVVVPRY-------GDYEEAYDVG----VRKYYKAA--GQDMEVNYFH | 71 |
| 690 | 6467503 | 285 | LPKALARRGHRVMVVAPRY-------GNYVEPQDTG----VRKRYKVD--GQDFEVSYFQ | 331 |
| 691 | 2833384 | 286 | LPKALARRGHRVMIVAPHY-------GNYAEAHDIG----VRKRYKVA--GQDMEVTYFH | 332 |
| 692 | 2129898 | 286 | LPKALARRGHRVMIVAPHY-------GNYAEAHDIG----VRKRYKVA--GQDMEVTYFH | 332 |
| 693 | 3192881 | 164 | LPKALARRGHRVMVVVPLY-------GNYAEPQHTG----VRKMFKID--GQDMEVNYFH | 210 |
| 694 | 8953571 | 332 | LPKALAKRGHRVMVVVPRY-------GDYEEAYDVG----VRKYYKAA--GQDMEVNYFH | 378 |
| 695 | 7529653 | 333 | LPKALAKRGHRVMVVVPRY-------GDYEEAYDVG----VRKYYKAA--GQDMEVNYFH | 379 |
| 696 | 8953571 | 333 | LPKALAKRGHRVMVVVPRY-------GDYEEAYDVG----VRKYYKAA--GQDMEVNYFH | 379 |
| 697 | 5825480 | 333 | LPKALAKRGHRVMVVVPRY-------GDYEEAYDVG----VRKYYKAA--GQDMEVNYFH | 379 |
| 698 | 14495348 | 283 | LPKALARRGURVMVVVPMY-------KNYAEPQQLG----EPRRYQVA--GQDMEVIYYH | 329 |
| 699 | 2833390 | 321 | LFKALARRGHRVMVVAPRY-------DNYPEPQDSG----VRKIYKVD--GQDVDVTYFQ | 367 |
| 700 | 8708896 | 110 | LPKALVRRGHRVMVVVPRY-------ENYDNAWETG----IRKIYSVF--NSNQEVGYFH | 156 |
| 701 | 5880466 | 164 | LPIALAARGHRVMVVMPRYLNGSSD-KNYAKALYTG----KHIKIPCF--GGSHEVTFFH | 216 |
| 702 | 9369334 | 164 | LPIALAARGHRVMVVMPRYLNGSSD-KNYAKALYTG----KHIKIPCF--GGSHEVTFFH | 216 |
| 703 | 15237934 | 167 | LPIALAGRGHRVMVISPRYLNGTAADKNYARAKDLG----IRVTVNCF--GGSQEVSFYH | 220 |
| 704 | 7188796 | 160 | LPIALAARGHRVMVVMPRYLNGTSD-KNYAKALYTG----KHIKIPCF--GGSHEVTFFH | 212 |
| 705 | 6103327 | 164 | LPIALAARGHRVMVVMPRYLNGSSD-KNYAKALYTA----KHIKIPCF--GGSHEVTFFH | 216 |
| 706 | 9369336 | 164 | LPIALAARGHRVMVVMPRYLNGSSD-KNYAKALYTA----YHIKIPCF--GGSHEVTFFH | 216 |
| 707 | 6690399 | 90 | LPIALAGRGHRVMVISPRYLNGTAADKNYAPAKDLG----IRVTVNCF--GGSQEVSFYH | 143 |
| 708 | 2829792 | 156 | LPMALAARGHRVMVVSPRYLNGGPSDEKYANAVDLD----VRATVHCF--GDAQEVAFYH | 209 |
| 709 | 2833387 | 18 | LPIALAARGHRVMVVMPRYLNGSSD-KNYAKALYTA----KHIKIPCF--GGSHEVTFFH | 70 |
| 710 | 12019656 | 164 | LPVALAARGHRVMVVMPRYLNGTSD-KNYANAFYTE----KHIRIPCF--GGEHEVTFFH | 216 |
| 711 | 7489712 | 157 | LPVALAARGHRVMVVMPRYLNGTSD-KNYANAFYTE----KHIRIPCF--GGEHEVTFFH | 209 |
| 712 | 5295947 | 158 | LPIALALRGHRVMVVMPRY-------MNGALNKNFANAFYTEKHIKIPCFGGEHEVTFFH | 210 |
| 713 | 1549232 | 158 | LPIALALRGHRVMVVMPRY-------MNGALNKNFANAFYTEKHIKIPCFGGEHEVTFFH | 210 |
| 714 | 2833377 | 158 | LPIALALRGHRVMVVMPRY-------MNGALNKNFANAFYTEKHIKIPCFGGEUEVTFFH | 210 |
| 715 | 6136121 | 107 | LPPAMAGNGHRVMTVSPRY-------DQYKDAWDTS----VVVEIKVG--DSIETVRFFH | 153 |
| 716 | 228210 | 106 | LPPALAARGHRVMTISPRY-------DQYKDAWDTS----VAVEVKVG--DSIEIVRFFH | 152 |
| 717 | 297196 | 106 | LPPALAARGHRVMTISPRY-------DQYKDAWDTS----VAVEVKVG--DSIEIVRFFH | 152 |
| 718 | 2833388 | 107 | LPPAMAARGHRVMTVSPRY-------DQYKDAWDTS----VSVEIKIG--DRIETVRFFH | 153 |
| 719 | 15637079 | 107 | LPPALAARGHRVMTVCPRY-------DQYKDAWDTC----VVVELQVG--DRIEPVRFFH | 153 |
| 720 | 602594 | 106 | LPPALAARGHRVMTISPRY-------DQYKDTWDTS----VAVEVKVG--DSIEIVRFFH | 152 |
| 721 | 2833381 | 107 | LPPALAARGHRVMTVCPRY-------DQYKDAWETC----VVVEPQVG--DRIEPVRFFH | 153 |
| 722 | 5441242 | 105 | LPSALAEHGHRVMTVSPRY-------DQYKDAWDTN----VTVEVKVA--DRIETVRFFH | 151 |
| 723 | 15223331 | 109 | LPPALAARGHRVMTICPRY-------DQYKDAWDTC----VVVQIKVG--DKVENVRFFH | 155 |
| 724 | 12003285 | 103 | LPPALAANRHRVMTVSPRY-------DQYKDAWDTS----VVVEIQVG--DKVETVGFFH | 149 |
| 725 | 3832512 | 106 | LPPALAGNGHRVMTVSPRY-------DQYKDAWDTG----VSVEIKVG--DRFETVRFFH | 152 |
| 726 | 15626365 | 112 | LPPALSANGHRVMTVTPRY-------DQYKDAWDTN----VTIEVKVG--DRTEKVRFFH | 158 |
| 727 | 2833383 | 102 | LPPVLAGNGHRVMTVSPRY-------DQYKDAWDTN----VLVEVKVG--DKIETVRFFH | 148 |
| 728 | 6624281 | 102 | LPAAMAANGHRVMVISPRY-------DQYKDAWDTS----VISEIKVV--DRYERVRYFH | 148 |
| 729 | 17736918 | 32 | LPPAMAANGHRVMVISPRY-------DQYKDAWDTS----VVSEIKVV--DKYERVRYFH | 78 |
| 730 | 4760584 | 102 | LPPAMAANGHRVMVISPRY-------DQYKDAWDTS----VVSEIKVV--DKYERVRYFH | 148 |
| 731 | 6492245 | 97 | LPPALAAMGHRVMTIAPRY-------DQYKDTWDTN----VLVEVIVG--DRTETVRFFH | 143 |
| 682 | MaizeSSIIb | 279 | SYIDGVDFVFVEAPPFRH---R---------HNNIY----G---G----ERLDILKRMIL | 315 |
| 683 | 7489711 | 279 | SYIDGVDFVFVEAPPFRH---R---------HNNIY----G---G----ERLDILKRMIL | 315 |
| 684 | 15028467 | 275 | AFIDGVDFVFLEAPPFRH---R---------HNDIY----G---G----ERFDVLKRMIL | 311 |
| 685 | 15384987 | 218 | AFIDGVDFVFLEAPPFRH---R---------HNDIY----G---G----ERFDVLKRMIL | 254 |
| 686 | 7489710 | 313 | AFIDGVDFVFIDAPLFRH---R---------QDDIY----G---G----SRQEIMKRMIL | 349 |
| 687 | 16265834 | 391 | AFIDGVDFVFIDAPLFRH---R---------QDDIY----G---G----NRQEIMKRMIL | 427 |
| 688 | 15232051 | 373 | AFIDGVDFVFIDSPEFRH---L---------SNNIY----G---G----NRLDILKRMVL | 409 |
| 689 | 7489695 | 72 | AYIDGVDFVFIDAPLFRH---R---------QEDIY----G---G----SRQEIMKRMIL | 108 |
| 690 | 6467503 | 332 | AFIDGVDFVFIDSPMFRH---I---------GNDIY----G---G----NRMDILKRMVL | 368 |
| 691 | 2833384 | 333 | TYIDGVDIVFIDSPIFRN---L---------ESNIY----G---G----NRLDILRRMVL | 369 |
| 692 | 2129898 | 333 | TYIDGVDIVFIDSPIFRN---L---------ESNIY----G---G----NRLDILRRMVL | 369 |
| 693 | 3192881 | 211 | AYIDNVDFVFIDSPIFQH---R---------GNNIY----G---G----NRVDILKRMDL | 247 |
| 694 | 8953571 | 379 | AYIDGVDFVFIDAPLFRH---R---------QEDIY----G---G----SRQEIMKRMIL | 415 |
| 695 | 7529653 | 380 | AYIDGVDFVFIDAPLFRH---R---------QEDIY----G---G----SRQEIMKRMIL | 416 |
| 696 | 8953571 | 380 | AYIDGVDFVFIDAPIFRH---R---------QEDIY----G---G----SRQEIMKRMIL | 416 |
| 697 | 5825480 | 380 | AYIDGVDFVFIDAPLFRH---R---------QEDIY----G---G----SRQEIMKRMIL | 416 |
| 698 | 14495348 | 330 | AYIDGVDFVFIDNPIFHH---V---------ENDIY----G---G----DRTDILKRMVL | 366 |
| 699 | 2833390 | 368 | ALLMDCDFVFIHSHMFRH---I---------GNNIY----G---G----NRVDILKRMVL | 404 |
| 700 | 8708896 | 157 | AFVDGVDYVFVDHPTFHG---R---------GKNIY----G---G----ERQEILFRCAL | 193 |
| 701 | 5880466 | 217 | EYRDNVDWVFVDHPSY-H---R---------PGSLY----GDNFG----AFGDNQFRYTL | 255 |
| 702 | 9369334 | 217 | EYRDNVDWVFVDHPSY-H---R---------PGSLY----GDNFG----AFGDNQFRYTL | 255 |
| 703 | 15237934 | 221 | EYRDGVDWVFVDHKSY-H---R---------PGNPY----GDSKG----AFGDNQFRFTL | 259 |
| 704 | 7188796 | 213 | EYRDNVDWVFVDHPSY-H---R---------PGSLY----GDNFG----AFGDNQFRYTL | 251 |
| 705 | 6103327 | 217 | EYRDNVDWVFVDHPSY-H---R---------PGSLY----GDNFG----AFGDNQFRYTL | 255 |
| 706 | 9369336 | 217 | EYRDNVDWVFVDHPSY-H---R---------PGSLY----GDNFG----AFGDNQFRYTL | 255 |
| 707 | 6690399 | 144 | EHRDGVDWVFVDHKSY-H---R---------PGNPY----GDSKG----AFGDNQFRFTL | 182 |
| 708 | 2829792 | 210 | EYRAGVDWVFVDHSSYCRPGTP---------YGDIY----G---A----FG-DNQFRFTL | 248 |
| 709 | 2833387 | 71 | EYRDNVDWVFVDUPSY-H---R---------PGSLY----GDNFG----AFGDNQFRYTL | 109 |
| 710 | 12019656 | 217 | EYRDSVDWVFVDHPSY-H---R---------PGNLY----GDKFG----AFGDNQFRYTL | 255 |
| 711 | 7489712 | 210 | EYRDSVDWVFVDNPSY-H---R---------PGNLY----GDKFG----AFGDNQFRYTL | 248 |
| 712 | 5295947 | 211 | EYRDSVDWVFVDHPSY-H---R---------PGNLY----GDNFG----AFGDNQFRYTL | 249 |
| 713 | 1549232 | 211 | EYRDSVDWVFVDNPSY-H---R---------PGNLY----GDNFC----AFGDNQFRYTL | 249 |
| 714 | 2833377 | 211 | EYRDSVDWVFVDHPSY-H---R---------PGNLY----GDNFG----AFGDNQFRYTL | 249 |
| 715 | 6136121 | 154 | CYKRGVDRVFVDHPIFLE---KVWGKT----KSKIYGPNAG---T----DYQDNQLRFSL | 199 |
| 716 | 228210 | 153 | CYKRGVDRVFVDHPMFLE---KVWGKT----GSKIY----G---PKAGLDYLDNELRFSL | 198 |
| 717 | 297196 | 153 | CYKRGVDRVFVDHPMFLE---KVWGKT----GSKIY----G---PKAGLDYLDNELRFSL | 198 |
| 718 | 2833388 | 154 | SYKRGVDRVFVDHPMFLE---KVWGKT----GSKIY----G---PRAGLDYQDNQLRFSL | 199 |
| 719 | 15637079 | 154 | SYKRGVDRVFVDHPMFLE---KVWGKTGSMLYGPKA----G---K----DYKDNQLRFSL | 199 |
| 720 | 602594 | 153 | CYKRGVDRVFVDHPMFLE---KVWGKT----GSKIY----G---PKAGLDYLDNELRFSL | 198 |
| 721 | 2833381 | 154 | SYKRGVDRVFVDHPMFLE---KVWGKTGSMLYGPKA----G---K----DYKDNQLRFSL | 199 |
| 722 | 5441242 | 152 | CYKQGVDRVFVDHPCFLE---KVWGKT----GSKLY----G---PSAGVDYEDNQLRYSL | 197 |
| 723 | 15223331 | 156 | CYKRGVDRVFVDHPIFLA---KVVGKTCSKIYGPIT----G---V----DYNDNQLRFSL | 201 |
| 724 | 12003285 | 150 | CYKRGVDRVFVDHPLFLE---KVWGKT----KSKVY----G---PSAGVDYEDNQLRFSL | 195 |
| 725 | 3832512 | 153 | CYKRGVDRVFVDHPLFLE---KVWGKT----ESKLY----G---PKTGVDYKDNQLRFSL | 198 |
| 726 | 15626365 | 159 | CFKRGVDRVFVDHPIFLE---KVWGKTGTKLYGPAA----G---D----DYQDNQLRFSI | 204 |
| 727 | 2833383 | 149 | CYKRGVDRVFVDHPLFLE---RVWGKT----GSKLY----G---PKTGIDYRDNQLRFSL | 194 |
| 728 | 6624281 | 149 | CYKRGVDRVFVDHPCFLE---KVRGKT----KEKIYGPDAG---T----DYEDNQQRFSL | 194 |
| 729 | 17736918 | 79 | CYKRGVDRVFVDHPCFLE---KVRGKT----KEKIYGPDAG---T----DYEDNQQRFSL | 124 |
| 730 | 4760584 | 149 | CYKRGVDRVFVDHPCFLE---KVRGKT----KEKIYGPDAG---T----DYEDNQQRFSL | 194 |
| 731 | 6492245 | 144 | CYKRGVDRVFVDHPMFLE---KVWGKTGSKLYGPTT----G---T----DFRDNQLRFCL | 189 |
| 682 | MaizeSSIIb | 316 | FCKAAVEVPWYA-----PCGGTV-----YGDG----NLVFIANDWHTALLPVYLKAYYRD | 361 |
| 683 | 7489711 | 316 | FCKAAVEVPWYA-----PCGGTV-----YGDG----NLVFIANDWHTALLPVYLKAYYRD | 361 |
| 684 | 15028467 | 312 | FCKAAVEVPWFA-----PCGGSI-----YGDG----NLVFIANDWHTALLPVCLKAYYRD | 357 |
| 685 | 15384987 | 255 | FCKAAVEVPWFA-----PCGGSI-----YGDG----NLVFIANDWHTALLPVYLKAYYRD | 300 |
| 686 | 7489710 | 350 | FCKVAVEVPWHV-----PCGGVC-----YGDG----NLVFIANDWHTALLPVYLKAYYRD | 395 |
| 687 | 16265834 | 428 | FCKAAVEVPWHV-----PCGGVP-----YGDG----NLVFLANDWHTALLPVYLKAYYRD | 473 |
| 688 | 15232051 | 410 | FCKAAVEVPWYV-----PCGGVC-----YGDG----NLAFIANDWHTALLPVYLKAYYRD | 455 |
| 689 | 7489695 | 109 | FCKAAVEVPWHV-----PCGGVP-----YGDG----NLVFIANDWHTALLPVYLKAYYRD | 154 |
| 690 | 6467503 | 369 | FCKAAVEVPWHV-----PCGGVC-----YGDG----NLAFIANDWHTALLPVYLKAYYRD | 414 |
| 691 | 2833384 | 370 | FCKAAVEVPWHV-----PCGGIC-----YGDG----NLVFIANDWHTALLPVYLKAYYRD | 415 |
| 692 | 2129898 | 370 | FCKAAVEVPWHV-----PCGGIC-----YGDG----NLVFIANDWHTALLPVYLKAYYRD | 415 |
| 693 | 3192881 | 248 | FCKAAIVVPWHV-----PCGGIC-----YGDG----NLVFIANDWHTALLPVYLKAYFRD | 293 |
| 694 | 8953573 | 416 | FCKAAVEVPWHV-----PCGGVP-----YGDG----NLVFIANDWHTALLPVYLKAYYRD | 461 |
| 695 | 7529653 | 417 | FCKAAVEVPWHV-----PCGGVP-----YGDG----NLVFIANDWHTALLPVYLKAYYRD | 462 |
| 696 | 8953571 | 417 | FCKAAVEVPWHV-----PCGGVP-----YGDG----NLVFIANDWHTALLPVYLKAYYRD | 462 |
| 697 | 5825480 | 417 | FCKAAVEVPWHV-----PCGGVP-----YGDG----NLVFIANDWHTALLPVYLKAYYRD | 462 |
| 698 | 14495348 | 367 | LCKAAIEVPWYV-----PCGGYC-----YGDG----NLVFLANDWHTALLPVYLKAYYHD | 412 |
| 699 | 2833390 | 405 | FCKAAIEVPWHV-----PCGGVC-----YGDG----NLVFIANDWHTALLPAYLKAYYRD | 450 |
| 700 | 8708896 | 194 | LCKAALEAVWHV-----PCGGIT-----YGDD----NLCFIANDWHTALLPVYLQAHYRD | 239 |
| 701 | 5880466 | 256 | LCYAACEAPLIL-----ELGGYI-----YGQ-----NCMFVVNDWHASLVPVLLAAKYRP | 300 |
| 702 | 9369334 | 256 | LCYAACEAPLIL-----ELGGYI-----YGQ-----NCMFVVNDWHASLVPVLLAAKYRP | 300 |
| 703 | 15237934 | 260 | LCHAACEAPLVL-----PLGGFT-----YGEK----SL-FLVNDWHAGLVPILLAAKYRP | 304 |
| 704 | 7188796 | 252 | LCYAACEAPLIL-----ELGGYI-----YGQ-----SCMFVVNDWHASLVPVLLAAKYRP | 296 |
| 705 | 6103327 | 256 | LCYAACEAPLIL-----ELGGYI-----YGQ-----NCMFVVNDWHASLVPVLLAAKYRP | 300 |
| 706 | 9369336 | 256 | LCYAACEAPLIL-----ELGGYI-----YGQ-----NCMFVVNDWHASLVPVLLAAKYRP | 300 |
| 707 | 6690399 | 183 | LCHAACEAPLVL-----PLGGFT-----YGEK----SL-FLVNDWHAGLVPILLAAKYRP | 227 |
| 708 | 2829792 | 249 | LSHAACEAPLVL-----PLGGFT-----YGEK----CL-FLANDWHAALVPLLLAAKYRP | 293 |
| 709 | 2833387 | 110 | LCYAACEAPLIL-----ELGGYI-----YGQ-----NCMFVVNDWHASLVPVLLAAKYRP | 154 |
| 710 | 12019656 | 256 | LCYAACEAPLVL-----ELGGYI-----YGQ-----NCMFVVNDWHASLVPVLLAAKYRP | 300 |
| 711 | 7489712 | 249 | LCYAACEAPLIL-----ELGGYI-----YGQ-----NCMFVVNDWHASLVPVLLAAKYRP | 293 |
| 712 | 5295947 | 250 | LCYAACEAPLIL-----ELGGYI-----YGQ-----KCMFVVNDWHASLVPVLLAAKYRP | 294 |
| 713 | 1549232 | 250 | LCYAACEAPLIL-----ELGGYI-----YGQ-----KCMFVVNDWHASLVPVLLAAKYRP | 294 |
| 714 | 2833377 | 250 | LCYAACEAPLIL-----ELGGYI-----YGQ-----KCMFVVNDWHASLVPVLLAAKYRP | 294 |
| 715 | 6136121 | 200 | LCQAALEAPRVL-----NLTSSK-----YFSGPYGEDVVFVANDWHTALLPCYLKSMYQS | 249 |
| 716 | 228210 | 199 | LCQAALEAPKVL-----NLNSSN-----YFSGPYGEDVLFIANDWHTALIPCYLKSMYQS | 248 |
| 717 | 297196 | 199 | LCQAALEAPKVL-----NLNSSN-----YFSGPYGEDVLFIANDWHTALIPCYLKSMYQS | 248 |
| 718 | 2833388 | 200 | LCLAALEAPRVL-----NLNSSKNFSGPYGE-----EVAFIANDWHTALLPCYLKAIYQP | 249 |
| 719 | 15637079 | 200 | LCQAALEAPRVL-----NLNSSN-----YFSGPYGEDVVFVANDWHTALLPCYLKTMYQS | 249 |
| 720 | 602594 | 199 | LCQAALEAPKVL-----NLSSSN-----YFSGPYGEDVLFIANDWHTALIPCYLKSMYQS | 248 |
| 721 | 2833381 | 200 | LCQAALEAPRVL-----NLNSSK-----YFSGPYGEDVVFVANDWHTALLPCYLKTMYQS | 249 |
| 722 | 5441242 | 198 | LCQAALEAPRVL-----NLNSNK-----YFSGPYGEDVIFVANDWHTALLPCYLKSMYQT | 247 |
| 723 | 15223331 | 202 | LCQAALEAPQVL-----NLNSSK-----YFSGPYGEDVVFVANDWHTALLPCYLKSMYQS | 251 |
| 724 | 12003285 | 196 | LSLAALEAPRVL-----NLTSNK-----YFSGPYGEDVVFVANDWHTAVLPCYLKTIYQP | 245 |
| 725 | 3832512 | 199 | LCQAALEAPRVL-----NLNSNK-----HFSGPYGEDVVFVANDWHTALLPCYLKSLYKS | 248 |
| 726 | 15626365 | 205 | FCQAALEAARVL-----NLKSNK-----YFSGPYGEDVIFVANDWHTALISCYMKSMYQS | 254 |
| 727 | 2833383 | 195 | LCQAALEAPRVL-----NLNSSK-----YFSGPYGEDVIFVANDWHSALIPCYLKSMYKS | 244 |
| 728 | 6624281 | 195 | LCQAALEVPRILDLNNNPHFSGP-----YGE-----DVVFVCNDWHTGLLACYLKSNYQS | 244 |
| 729 | 17736918 | 125 | LCQAALEVPRIL-----NLDNNP-----YFSGPYGEDVVFVCNDWHTGLLACYLKSNYQS | 174 |
| 730 | 4760584 | 195 | LCQAALEVPRIL-----NLDNNP-----YFSGPYGEDVVFVCNDWHTGLLACYLKSNYQS | 244 |
| 731 | 6492245 | 190 | LCLAALEAPRVL-----NLNNSE-----YFSGPYGENVVFVANDWHTAVLPCYLKSMYKQ | 239 |
| 682 | MaizeSSIIb | 362 | NGLMQYARSVLVIHNIAHQGRGPVDDFVNFDLPEHYIDHF--------KLYDN----IG- | 408 |
| 683 | 7489711 | 362 | NGLMQYARSVLVIHNIAHQGRGPVDDFVNFDLPEHYIDHF--------KLYDN----IG- | 408 |
| 684 | 15028467 | 358 | NGLMQYTRSVLVIHNIAHQGRGPVDDFATMDLPEHYIDHF--------RLYDP----VG- | 404 |
| 685 | 15384987 | 301 | NGLMQYTRSVLVIHNIAHQGRGPVDDFATMDLPEHYIDHF--------RLYDP----VG- | 347 |
| 686 | 7489710 | 396 | HGLMQYTRSVLVIHNIAHQGRGPVDEFPYMDLPEHYLQRF--------ELYDP----VG- | 442 |
| 687 | 16265834 | 474 | NGMMQYTRSVLVIHNIAYQGRGPVDEFPYMELPEHYLDHF--------KLYDP----VG- | 520 |
| 688 | 15232051 | 456 | HGIMKYTRSVLVIHNIAHQGRGPVDDFSYVDLPSHYLDSF--------KLYDP----VG- | 502 |
| 689 | 7489695 | 155 | HGLMQYTRSIMVIHNIAHQGRGPVDEFPFTELPEHYLEHF--------RLYDP----VG- | 201 |
| 690 | 6467503 | 415 | NGLMQYTRSVLVIHNIAHQGRGPSGDFSYVGLPEHYIDLF--------KLHDP----IG- | 461 |
| 691 | 2833384 | 416 | HGLMNYTRSVLVIHNIAHQGRGPVEDFNTVDLSGNYLDLF--------KMYDP----VG- | 462 |
| 692 | 2129898 | 416 | HGLMNYTRSVLVIHNIAHQGRGPVEDFNTVDLSGNYLDLF--------KMYDP----VG- | 462 |
| 693 | 3192881 | 294 | NGVMKFTRSVLVIHNIAHQGRGPMDDFSIVDLPAQYADLF--------KLYDP----VG- | 340 |
| 694 | 8953573 | 462 | HGLMQYTRSIMVIHNIAHQGRGFVDEFPFTELPEHYLEHF--------RLYDP----VG- | 508 |
| 695 | 7529653 | 463 | HGLMQYTRSIMVIHNIAHQGRGPVDEFPFTELPEHYLEHF--------RLYDP----VG- | 509 |
| 696 | 8953571 | 463 | HGLMQYTRSIMVIHNIAHQGRGPVDEFPFTELPEHYLEHF--------RLYDP----VG- | 509 |
| 697 | 5825480 | 463 | HGLMQYTRSIMVIHNIAHQGRGPVDEFPFTELPEHYLEHF--------RLYDP----VG- | 509 |
| 698 | 14495348 | 413 | NGFMIYARSVLVIHNIAHQGRGPLDDFSYLDLPVDYMDLF--------KLYDP----FG- | 459 |
| 699 | 2833390 | 451 | NGIMNYTRSVLVIHNIAHQGRGPLEDFSYVDLPPHYMDPF--------KLYDF----VG- | 497 |
| 700 | 8708896 | 240 | YGEMTYARCAFVIHNMAHQGRGPFVESEHLELNEEYRERF--------RLYDP----IG- | 286 |
| 701 | 5880466 | 301 | YGVYRDSRSTLVIHNLAHQGLEPASTYPDLGLPPEWYGALEWVFPEWARRHAL----DK- | 355 |
| 702 | 9369334 | 301 | YGVYRDSRSTLVIHNLAHQGVEPASTYPDLGLPFEWYGALEWVFPEWARRHAL----DK- | 355 |
| 703 | 15237934 | 305 | YGVYKDARSILIIHNLAHQGVEPAATYTNLGLPSEW--------------YGA----VGW | 346 |
| 704 | 7188796 | 297 | YGVYRDSRSTLVIHNLAHQGVEPASTYPDLGLPPEWYGALEWVFPEWARRHAL----DK- | 351 |
| 705 | 6103327 | 301 | YGVYRDSRSTLVIHNLAHQGVEPASTYPDLGLPPEWYGALEWVFPEWARRHAL----DK- | 355 |
| 706 | 9369336 | 301 | YGVYRDSRSTLVIHNLAHQGVEPASTYPDLGLPPEWYGALEWVFPEWARRHAL----DK- | 355 |
| 707 | 6690399 | 228 | YGVYKDARSILIIHNLAHQGVEPAATYTNLGLPSEW--------------YGA----VGW | 269 |
| 708 | 2829792 | 294 | YGVYKDARSIVAIHNIAHQGVEPAVTYNNLGLPPQWYGAVEWIFPTWARAHAL----DT- | 348 |
| 709 | 2833387 | 155 | YGVYRDSRSTLVIHNLAHQGVEPASTYPDLGLPPEWYGALEWVFPEWARRHAL----DK- | 209 |
| 710 | 12019656 | 301 | YGVYKDSRSILVIHNLAHQGVEPASTYPDLGLPPEWYGALEWVFPEWARRHAL----DK- | 355 |
| 711 | 7489712 | 294 | YGVYKDSRSILVIHNLAHQGVEPASTYPDLGLPPEWYGALEWVFPEWARRHAL----DK- | 348 |
| 732 | 7484400 | 4 | RGRGPFVESEHLELNEEYRERF--------RLYDP----IG- | 32 |
| 712 | 5295947 | 295 | YGVYRDARSVLVIHNLAHQGVEPASTYPDLGLPPEWYGALEWVFPEWARRHAL----DK- | 349 |
| 713 | 1549232 | 295 | YGVYRDARSVLVIHNLAHQGVEPASTYPDLGLPPEWYGALEWVFPEWARRHAL----DK- | 349 |
| 714 | 2833377 | 295 | YGVYRDARSVLVIHNLAHQGVEPASTYPDLGLPPEWYGALEWVFPEWARRHAL----DK- | 349 |
| 715 | 6136121 | 250 | KGMYLHAKVAFCIHNIAYQGRFGSSDFCLLNLPDQFKSSF--------DFFDGYEKPVK- | 300 |
| 716 | 228210 | 249 | RGIYLNAKVAFCIHNIAYQGRFSFSDFPLLNLPDEFRGSF--------DFIDGYEKPVK- | 299 |
| 717 | 297196 | 249 | RGIYLNAKVAFCIHNIAYQGRFSFSDFPLLNLPDEFRGSF--------DFIDGYEKPVK- | 299 |
| 718 | 2833388 | 250 | MGIYKHAKVAFCIHNIAYQGRFAFSDFPRLNLPDKFKSSF--------DFIDGYEKPVK- | 300 |
| 719 | 15637079 | 250 | RGIYMNAKVAFCIHNIAYQGRFAFSDFSLLNLPDEYKGSF--------DFIDGYDKPVK- | 300 |
| 720 | 602594 | 249 | RGIYLNAKVAFCIHNIAYQGRFSFSDFPLLNLPDEFRGSF--------DFIDGYEKPVK- | 299 |
| 721 | 2833381 | 250 | RGIYMNAKVAFCIHNIAYQGRFAFSDFSLLNLPDEYKGSF--------DFIDGYDKPVK- | 300 |
| 722 | 5441242 | 248 | RGVYRNTKVAFCIHNISYQGRHPFEDFPLLNLPNEYRSAF--------DFTDGHLKPVR- | 298 |
| 723 | 15223331 | 252 | RGVYMNAKVVFCIHNIAYQGRFAFDDYSLLNLPISFKSSF--------DFMDGYEKPVK- | 302 |
| 724 | 12003285 | 246 | KGIYTNAKVVLCIHNIAYQGRFAFSDFYKLNLPDQLKSSF--------DFMDGYEKPVK- | 296 |
| 725 | 3832512 | 249 | KGIYKSAKVAFCIHNIAYQGRHAFSDLSLLNLPNEFRSSF--------DFIDGYDKPVK- | 299 |
| 726 | 15626365 | 255 | IGIFRNAKVVFCIHNIAYQGRFAFTDYSLLNLPDQFKSSF--------DFLDGHVKPIV- | 305 |
| 727 | 2833383 | 245 | RGLYKNAKVAFCIHNIAYQGRNAFSDFSLLNLPDEFRSSF--------DFIDG----YNK | 292 |
| 728 | 6624281 | 245 | NGIYRTAKVAFCIHNISYQGRFSFDDFAQLNLPDRFKSSF--------DFIDGYDKPVE- | 295 |
| 729 | 17736918 | 175 | NGIYRAAKVAFCIHNISYQGRFSFDDFAQLNLPDRFKSSF--------DFIDGYDKPVE- | 225 |
| 730 | 4760584 | 245 | NGIYRAAKVAFCIHNISYQGRFSFDDFAQLNLPDRFKSSF--------DFIDGYDKPVE- | 295 |
| 731 | 6492245 | 240 | NGIYVNAKVAFCIHNIAYQGRFPRVDFELLNLPESFMPSF--------DFVDGHVKPVV- | 290 |

TABLE XXVII
Maize soluble starch synthase IIb (SSIIb)
“LINKR” Domain Alignments with other similar proteins

| SEQ | Accession | a.a | | a.a. |
|---|---|---|---|---|
| Id.No. | Number | # | (start) Sequence end | # |
| 733 | MaizeSSIIb | 409 | -------------GDHSNVFAAGLKTADRVVTVSNGYMWELKT-S-EG-GWGLHDIINQN | 452 |
| 734 | 7489711@ | 409 | -------------GDHSNVFAAGLKTADRVVTVSNGYMWELKT-S-EG-GWGLHDIINQN | 452 |
| 735 | 15028467@ | 405 | -------------GEHSNVFAAGLKMADRAVTVSHGYLWEIKT-M-DG-GWGLHEIINHN | 448 |
| 736 | 15384987@ | 348 | -------------GEHSNVFAAGLKMADRAVTVSHGYLWEIKT-M-DG-GWGLHEIINHN | 391 |
| 737 | 7489710@ | 443 | -------------GEHANIFAAGLKMADRVVTVSRGYLWELKT-V-EG-GWGLHDIIRSN | 486 |
| 738 | 16265834@ | 521 | -------------GEHANIFGAGLKMADRVVTVSPGYLWELKT-T-EG-GWGLHDIIREN | 564 |
| 739 | 15232051@ | 503 | -------------GEHFNIFAAGLKAADRVLTVSHGYSWEVKT-L-EG-GWGLHNIINEN | 546 |
| 740 | 7489695@ | 202 | -------------GEHANYFAAGLKMADQVVVVSPGYLWELKT-V-EG-GWGLHDIIRQN | 245 |
| 741 | 6467503@ | 462 | -------------GDHFNIFAPGLKVADRVVTVSHGYAWELKT-S-EG-GWGLHNIINEN | 505 |
| 742 | 2833384@ | 463 | -------------GEHFNIFAAGLKTADRIVTVSHGYAWELKT-S-EG-GWGLHNIINES | 506 |
| 743 | 2129898@ | 463 | -------------GEHFNIFAAGLKTADRIVTVSHGYAWELKT-S-EG-GWGLHNIINES | 506 |
| 744 | 2192881@ | 341 | -------------GDHFNIFAAGLKTADRVVTVSHGYAWELKT-S-EG-GWGLNGIRNEN | 384 |
| 745 | 8953573@ | 509 | -------------GEHANYFAAGLKMADQVVVVSPGYLWELKT-V-EG-GWGLHDIIRQN | 552 |
| 746 | 7529653@ | 510 | -------------GEHANYFAAGLKMADQVVVVSPGYLWELKT-V-EG-GWGLHDIIRQN | 553 |
| 747 | 8953571@ | 510 | -------------GEHANYFAAGLKMADQVVVVSPGYLWELKT-V-EG-GWGLHDIIRQN | 553 |
| 748 | 5825480@ | 510 | -------------GEHANYFAAGLKMADQVVVVSPGYLWELKT-V-EG-GWGLHDIIRQN | 553 |
| 749 | 14495348@ | 460 | -------------GDHLNIFAAGIKAADRLLTVSHGYAWELKT-A-EG-GWGLHGIINES | 503 |
| 750 | 2833390@ | 498 | -------------GEHFNIFAAGLKTADRVVTVSHGYSWELKT-S-QG-GWGLHQIINEN | 541 |
| 751 | 8708896@ | 287 | -------------GEHMNVMKAGLECAHRLVAVSKCYAWECQT-V-EG-GWGLHEVIKVN | 330 |
| 752 | 5880466@ | 356 | -------------GEAVNFLKGAVVTADRIVTVSQGYSWEVTT-A-EG-GQGLNELLSSR | 399 |
| 753 | 9369334@ | 356 | -------------GEAVNFLKGAVVTADRIVTVSQGYSWEVTT-A-EG-GQGLNELLSSR | 399 |
| 754 | 15237934@ | 347 | VFPTWARTHALDTGEAVNVLKGAIVTSDRIITVSQGYAWEITT-V-EG-GYGLQDLLSSR | 403 |
| 755 | 7188796@ | 352 | -------------GEAVNFLKGAVVTADRIVTVSQGYSWEVTT-A-EG-GQGLNELLSSR | 395 |
| 756 | 6103327@ | 356 | -------------GEAVNFLKGAVVTADRIVTVSQGYSWEVTT-A-EG-GQGLNELLSSR | 399 |
| 757 | 9369336@ | 356 | -------------GEAVNFLKGAVVTADRIVTVSQGYSWEVTT-A-EG-GQGLNELLSSR | 399 |
| 758 | 6690399@ | 270 | VFPTWARTHALDTGEAVNVLKGAIVTSDRIITVSQGYAWEITT-V-EG-GYGLQDLLSSR | 326 |
| 759 | 2829792@ | 349 | -------------GETVNVLKGAIAVADRILTVSQGYSWEITT-P-EG-GYGLHELLSSR | 392 |
| 760 | 2833387@ | 210 | -------------GEAVNFLKGAVVTADRIVTVSQGYSWEVTT-A-EG-GQGLNELLSSR | 253 |
| 761 | 12019656@ | 356 | -------------GEAVNFLKGAVVTADRIVTVSKGYSWEVTT-A-EG-GQGLNELLSSR | 399 |
| 762 | 7489712@ | 349 | -------------GEAVNFLKGAVVTADRIVTVSKGYSWEVTT-A-EG-GQGLNELLSSR | 392 |
| 763 | 7484400@ | 33 | -------------GEHMNVMKAGLECAHRLVAVSKCYAWECQT-V-EG-GWGLHEVIKVN | 76 |
| 764 | 5295947@ | 350 | -------------GEAVNFLKGAVVTADRIVTVSQGYSWEVTT-A-EG-GQGLNELLSSR | 393 |
| 765 | 1549232@ | 350 | -------------GEAVNFLKGAVVTADRIVTVSQGYSWEVTT-A-EG-GQGLNELLSSR | 393 |
| 766 | 2833377@ | 350 | -------------GEAVNFLKGAVVTADRIVTVSQGYSWEVTT-A-EG-GQGLNELLSSR | 393 |
| 767 | 6136121@ | 301 | -------------GRKINWMKAGILESDRVVTVSPYYAMELVSGA-EK-GVELDNVIAKT | 345 |
| 768 | 228210@ | 300 | -------------GRKINWMKAGILESHRVVTVSPYYAQELVS-AVDK-GVELDSVLRKT | 344 |
| 769 | 297196@ | 300 | -------------GRKINWMKAGILESHRVVTVSPYYAQELVS-AVDK-GVELDSVLRKT | 344 |
| 770 | 2833388@ | 301 | -------------GRKINWMKAGILESDRVLTVSPYYAQEVIS-GVER-GVELDNFIRKT | 345 |
| 771 | 15637079@ | 301 | -------------GRKINWMKAGIREADRVFTVSPNYAKELVS-CVSK-GVELDNHI--R | 343 |
| 772 | 602594@ | 300 | -------------GRKINWMKAGILESHRVVTVSPYYAQELVS-AVDK-GVELDSVLRKT | 344 |
| 773 | 2833381@ | 301 | -------------GRKINWMKAGIREADRVFTVSPNYAKELVS-CVSK-GVELDNHI--R | 343 |
| 774 | 5441242@ | 299 | -------------GRKINWMKAAILESDLVLTVSPYYARELVS-G-EDRGVELDNIIRKT | 343 |
| 775 | 15223331@ | 303 | -------------GRKINWMKAAILEAHRVLTVSPYYAQELIS-GVDR-GVELHKYLRMK | 347 |
| 776 | 12003285@ | 297 | -------------GRKINWMKAGIIESDRVLTVSPYYANELVS-GPDK-GVELDNILRK- | 340 |
| 777 | 3832512@ | 300 | -------------GRKINWMKAGVLESDRVFTVSPYYAKELVS-G-EDRGVELDNIIRS- | 343 |
| 778 | 15626365@ | 306 | -------------GRKINWMKAGIIESHRVLTVSPYYAQELVS-GPDK-GVELDNILRRV | 350 |
| 779 | 2833383@ | 293 | PCE----------GKKINWMKAGILESDQVFTVSPHYAKELIS-G-EDRGVELDNIIRST | 340 |
| 780 | 6624281@ | 296 | -------------GRKINWMKAGILQADKVLTVSPYYAEELIS-G-EARGCGLDNIMRLT | 340 |
| 781 | 17736918@ | 226 | -------------GRKINWMKAGILQADKVLTVSPYYAEELIS-G-ETRGCELDNIMRLT | 270 |
| 782 | 4760584@ | 296 | -------------GRKINWMKAGILQADKVLTVSPYYAEELIS-G-EARGCELDNIMRLT | 340 |
| 783 | 6492245@ | 291 | -------------GRKINWMKAGITECDVVLTVSPHYVKELTS-GPEK-GVELDGVLRAK | 335 |
| 733 | MaizeSSIIb | 453 | DWKLQGIVNGIDMSEWNPAVD----VHL----HSDDYTN--YTFETLDTGKRQCKAALQR | 502 |
| 734 | 7489711@ | 453 | DWKLQGIVNGIDMSEWNPAVD----VHL----HSDDYTN--YTFETLDTGKRQCKAALQR | 502 |
| 735 | 15028467@ | 449 | DWKLQGIVNGIDMAEWNPEVD----EHL----QSDGYAN--YTFETLDTGKKQCKEALQR | 498 |
| 736 | 15384987@ | 392 | DWKLQGIVNGIDMAEWNPEVD----EHL----QSDGYAN--YTFETLDTGKKQCKEALQR | 441 |
| 737 | 7489710@ | 487 | DWKINGIVNGIDHQEWNPKVD----VHL----RSDGYTN--YSLETLDAGKRQCKAALQR | 536 |
| 738 | 16265834@ | 565 | DWKMNGIVNGIDYREWNPEVD----VHL----QSDGYAN--YTVASLDSSKPRCKAALQR | 614 |
| 739 | 15232051@ | 547 | DWKFRGIVNGIDTQEWNPEFD----TYL----HSDDYTN--YSLENLHIGKPQCKAALQK | 596 |
| 740 | 7489695@ | 246 | DWKTRGIVNGIDNMEWNPEVD----AHL----KSDGYTN--FSLRTLDSGKRQCKEALQR | 295 |
| 741 | 6467503@ | 506 | HWKLQGIVNGIDAKEWNPQFD----IQL----TSDGYTN--YSLETLDTGKPQCKTALQN | 555 |
| 742 | 2833384@ | 507 | DWKFRGIVNGVDTKDWNPQFD----AYL----TSDGYTN--YNLKTLQTGKRQCKAALQR | 556 |
| 743 | 2129898@ | 507 | DWKFRGIVNGVDTKDWNPQFD----AYL----TSDGYTN--YNLKTLQTGKRQCKAALQR | 556 |
| 744 | 3192881@ | 385 | EWKLQGIVNGIDIEEWNPQLD----VYL----KSDGYAE--YSLDTLQTGKPQCKAALQK | 434 |
| 745 | 8953573@ | 553 | DWKTRGIVEGIDNMEWNPEVD----VHL----KSDGYTN--FSLGTLDSGKRQCKEALQR | 602 |
| 746 | 7529653@ | 554 | DWKTRGIVNGIDNMEWNPEVD----AHL----KSDGYTN--FSLRTLDSGKRQCKEALQR | 603 |
| 747 | 8953571@ | 554 | DWKTRGIVNGIDNMEWNPEVD----VHL----QSDGYTN--FSLSTLDSGKRQCKEALQR | 603 |
| 748 | 5825480@ | 554 | DWKTRGIVNGIDNMEWNPEVD----VHL----KSDGYTN--FSLGTLDSGKRQCKEALQR | 603 |
| 749 | 14495348@ | 504 | DWKFQGIVNGIDTTDWNPRCD----IHL----KSDGYTN--YSLETVQAGKQQCKAALQK | 553 |
| 750 | 2833390@ | 542 | DWKLQGIVNGIDTKEWNPELD----VHLP---RSDGYMN--YSLDTLQTGKPQCKAALQK | 592 |
| 751 | 8708896@ | 331 | NWKLRGIVNGIDYKEWNPICD----EFL----TTDGYAH--YDVDTLAEGKAKCKAALQK | 380 |
| 752 | 5880466@ | 400 | KSVLNGIVNGIDINDWNPTTD----KCL----PE----H--YSVDDL-SGKAKCKAELQK | 444 |
| 753 | 9369334@ | 400 | KSVLNGIVNGIDINDWNPTTD----KCL----PH----H--YSVDDL-SGKAKCKAELQK | 444 |
| 754 | 15237934@ | 404 | KSVINGITNGINVDEWNPSTD----EHIPFHYSADDVSE-----------KIKCKMALQK | 448 |
| 755 | 7188796@ | 396 | KSVLNGIVNGIDINDWNPTTD----KCL----PH----H--YSVDDL-SGKAKCKAELQR | 440 |
| 756 | 6103327@ | 400 | KSVLNGIVNGIDINDWNPTTD----KCL----PH----H--YSVDDL-SGKAKCKAELQK | 444 |
| 757 | 9369336@ | 400 | KSVLNGIVNGIDINDWNPTTD----KCL----PH----H--YSVDDL-SGKAKCKAELQK | 444 |
| 758 | 6690399@ | 327 | KSVINGITNGINVDEWNPSTD----EHIPFHYSADDVSE-----------KIKCKMALQK | 371 |
| 759 | 2829792@ | 393 | QSVLNGITNGIDVNDWNPSTD----EHI----AS----H--YSINDL-SGKVQCKTDLQK | 437 |
| 760 | 2833387@ | 254 | KSVLNGIVNGIDINDWNPTTD----KCL----PH----H--YSVDDL-SGKAKCKAELQK | 298 |
| 761 | 12019656@ | 400 | KSVLNGIVNGIDINDWNPATDKCIPCHY----SVDDL-----------SGKAKCKSALQK | 444 |
| 762 | 7489712@ | 393 | KSVLNGIVNGIDINDWNPATDKCIPCHY----SVDDL-----------SGKAKCKGALQK | 437 |
| 763 | 7484400@ | 77 | NWKLRGIVNGIDYKEWNPICD----EFL----TTDGYAH--YDVDTLAEGKAKCKAALQK | 126 |
| 764 | 5295947@ | 394 | KSVLNGIVNGIDINDWNPSTD----KFL----P----YH--YSVDDL-SGKAKCKAELQK | 438 |
| 765 | 1549232@ | 394 | KSVLNGIVNGIDINDWNPSTD----KFL----P----YH--YSVDDL-SGKAKCKAELQK | 438 |
| 766 | 2833377@ | 394 | KSVLNGIVNGIDINDWNPSTD----KFL----P----YH--YSVDDL-SGKAKCKAELQK | 438 |
| 767 | 6136121@ | 346 | --SITGIVNGMDTQEWNPATD----KHI----D----TN--YDITTVMDAKPLLKEALQA | 389 |
| 768 | 228210@ | 345 | --CITGIVNGMDTQEWNPA--------------TDKYTDVKYDITTVMDAKPLLKEALQA | 388 |
| 769 | 297196@ | 345 | --CITGIVNGMDTQEWNPA--------------TDKYTDVKYDITTVMDAKPLLKEALQA | 388 |
| 770 | 2833388@ | 346 | G--IAGIINGMDVQEWNPVTD----KYI----D----IH--YDATTVMDAKPLLKEALQA | 389 |
| 771 | 15637079@ | 344 | DCGITGICNGMDTQEWNPATD----KYL----A----VK--YDITTVEQAKPLLKEALQA | 389 |
| 772 | 602594@ | 345 | --CITGIVNGMDTQEWNPA--------------TDKYTDVKYDITTVMDAKPLLKEALQA | 388 |
| 773 | 2833381@ | 344 | DCGITGICNGMDTQEWNPATD----KYL----A----VK--YDITTVMQAKPLLKEALQA | 389 |
| 774 | 5441242@ | 344 | G--VAGIVNGMDIREWSPKTDKF--IDI----HFDT--------TSVKEAKFLLKEALQA | 387 |
| 775 | 15223331@ | 348 | --TVSGIINGMDVQEWNPSTD----KYI----D----IK--YDITTVTDAKPLIKEALQA | 391 |
| 776 | 12003285@ | 341 | -CTVTGIVNGMDTQEWNPATD----KYI----DN----H--YDITTVMDGKPLLKEALQA | 385 |
| 777 | 3832512@ | 344 | -IGITGIVNGMDNREWSPQTD----RYI----D----VH--YDASTVTEAKAILKEALQA | 388 |
| 778 | 15626365@ | 351 | G--VTGIVNGMDVQEWNPSTD----KYI----S----IK--YDASTVLEGKALLKEELQA | 394 |
| 779 | 2833383@ | 341 | G--IIGIVNGMDNREWSPQTD----RYI----D----VH--YNETTVTEAKPLLKGTLQA | 384 |
| 780 | 6624281@ | 341 | G--ITGIVNGMDVSEWDPIKD----KFL--------TVN--YDVTTALEGKALNKEALQA | 384 |
| 781 | 17736918@ | 271 | G--ITGIVNGMDVSEWDPTKD----KFL----A----VN--YDITTALEGKALNKEALQA | 314 |
| 782 | 4760584@ | 341 | G--ITGIVNGMDVSEWDPTKD----KFL----A----VN--YDITTALEGKALNKEALQA | 384 |
| 783 | 6492245@ | 336 | PLE-TGIVNGMDVVDWNPATD----KYI----S----VK--YNATTVAEARALNKEILQA | 380 |

TABLE XXVIII
Maize soluble starch synthase IIb (SSIIb)
“GLYTR” Domain Alignments with other similar Proteins

| SEQ | Accession | a.a | | a.a. |
|---|---|---|---|---|
| Id.No. | Number | # | (start) Sequence end | # |
| 784 | MaizeSSIIa | 503 | QLGLQVRDDVPLIGFIGRLDHQKGVDIIADAIHWI-AG-QDVQLVMLGTGRADLEDMLRR | 560 |
| 785 | 7489711 | 503 | QLGLQVRDDVPLIGFIGRLDHQKGVDIIADAIHWI-AG-QDVQLVMLGTGRADLEDMLRR | 560 |
| 786 | 15028467 | 499 | QLGLQVRDDVPLIGFIGRLDHQKGVDIIGDAMPWI-AG-QDVQVVMLGTGRPDLEEMLRR | 556 |
| 787 | 15384987 | 442 | QLGLQVRDDVPLIGFIGRLDHQKGVDIIGDAMPWI-AG-QDVQVVMLGTGRPDLEEMLRR | 499 |
| 788 | 7489710 | 537 | ELGLEVRDDVPLLGFIGRLDGQKGVDIIGDAMPWI-AG-QDVQLVMLGTGRADLERMLQH | 594 |
| 789 | 16265834 | 615 | ELGLEVRDDVPLIGFIGRLDGQKGVDIIGDAMPWI-AG-QDVQLVLLGSGRRDLEVMLQR | 672 |
| 790 | 15232051 | 597 | ELGLPVRPDVPLIGFIGRLDHQKGVDLIAEAVPWM-MS-QDVQLVMLGTGRPDLEEVLRQ | 654 |
| 791 | 7489695 | 296 | ELGLQVRADVPLLGFIGRLDGQKGVEIIADAMPWI-VS-QDVQLVMLGTGRHDLESMLQH | 353 |
| 792 | 6467503 | 556 | ELRFAIPPDVPVIGFIGRLDYQKGVDLIAEAIPWM-VG-QDVQLVMLGTGRQDLEEMLRQ | 613 |
| 793 | 2833384 | 557 | ELGLPVREDVPIISFIGRLDHQKGVDLIAEAIPWM-MS-HDVQLVMLGTGRADLEQMLKE | 614 |
| 794 | 2129898 | 557 | ELGLPVREDVPIISFIGRLDHQKGVDLIAEAIPWM-MS-HDVQLVMLGTGRADLEQMLKE | 614 |
| 795 | 3192881 | 435 | EMNLPVRDDVPLIGFIGRLDHQKGVDLIAEAIPWM-MG-QDVQLVMLGTGRPDLEQMLKQ | 492 |
| 796 | 8953573 | 603 | ELGLQVRGDVPLLGFIGRLDGQKGVEIIADAMPWI-VS-QDVQLVMLGTGRHDLEGMLRH | 660 |
| 797 | 7529653 | 604 | ELGLQVRADVPLLGFIGRLDGQKGVEIIADAMPWI-VS-QDVQLVMLGTGRHDLESMLQH | 661 |
| 798 | 8953571 | 604 | ELGLQVRADVPLLGFIGRLDGQKGVEIIADAMPWI-VS-QDVQLVMLGTGRHDLESMLRH | 661 |
| 799 | 5825480 | 604 | ELGLQVRADVPLLGFIGRLDGQKGVEIIADAMPWI-VS-QDVQLVMLGTGRHDLESMLRH | 661 |
| 800 | 14495348 | 554 | ELGLPVRGDVPVIAFIGRLDHQKGVDLIAEAMPWI-AG-QDVQLIMLGTGRQDLEDTLRR | 611 |
| 801 | 2833390 | 593 | ELGLPVRDDVPLIGFIGRLDPQKGVDLIAEAVPWM-MG-QDVQLVMLGTGRRDLEQMLRQ | 650 |
| 802 | 8708896 | 381 | ELGLPVDPDAPMLGFIGRLDYQKGVDLIRDNYDYI-MG-EKCQLVMLGSGRQDLEDALRD | 438 |
| 803 | 5880466 | 445 | ELGLPVREDVPLIGFIGRLDYQKGIDLIKMAIPEL-MR-EDVQFVMLGSGDPIFEGWMRS | 502 |
| 804 | 9369334 | 445 | ELGLPVREDVPLIGFIGRLDYQKGIDLIKMAIPEL-MR-EDVQFVMLGSGDPIFEGWMRS | 502 |
| 805 | 15237934 | 449 | ELGLPIRPECPMIGFIGRLDYQKGIDLIQTAGPDL-MV-DDIQFVMLGSGDPKYESWMRS | 506 |
| 806 | 7188796 | 441 | ELGLPVREDVPLIGFIGRLDYQKGIDLIKMAIPDL-MR-EDVQFVMLGSGDPVFEGWMRS | 498 |
| 807 | 6103327 | 445 | ELGLPVREDVPLIGFIGRLDYQKGIDLIKMAIPEL-MR-EDVQFVMLGSGDPIFEGWMRS | 502 |
| 808 | 9369336 | 445 | ELGLPVREDVPLIGFIGRLDYQKGIDLIKMAIPEL-MR-EDVQFVMLGSGDPIFEGWMRS | 502 |
| 809 | 6690399 | 372 | ELGLPIRPECPMIGFIGRLDYQKGIDLIQTAGPDL-MV-DDIQFVMLGSGDPKYESWMRS | 429 |
| 810 | 2829792 | 438 | ELGLPIRPDCPLIGFIGRLDYQKGVDIILSAIPEL-MQ-NDVQVVMLGSGEKQYEDWMRH | 495 |
| 811 | 2833387 | 299 | ELGLPVREDVPLIGFIGRLDYQKGIDLIKMAIPEL-MR-EDVQFVMLGSGDPIFEGWMRS | 356 |
| 812 | 12019656 | 445 | ELGLPIRPEVPLIGFIGRLDYQKGIDLIQLIIPHL-MR-DDVQFVMLGSGDPELEDWMRS | 502 |
| 813 | 7489712 | 438 | ELGLPIRPDVFLIGFIGRLDYQKGIDLIQLIIPDL-MR-EDVQFVMLGSGDPELEDWMRS | 495 |
| 814 | 7484400 | 127 | ELGLPVDPDAPMLGFIGRLDYQKGVDLIRDNYDYI-MG-EKCQLVMLGSGRQDLEDALRD | 184 |
| 815 | 5295947 | 439 | ELGLPIRPDVPLIGFIGRLDYQKGIDLIKLAIPDL-MR-DNIQFVMLGSGDPGFEGWMRS | 496 |
| 816 | 1549232 | 439 | ELGLPIRPDVPLIGFIGRLDYQKGIDLIKLAIPDL-MR-DNIQFVMLGSGDPGFEGWMRS | 496 |
| 817 | 2833377 | 439 | ELGLPIRPDVPLIGFIGRLDYQKGIDLIKLAIPDL-MR-DNIQFVMLGSGDPGFEGWMRS | 496 |
| 818 | 6136121 | 390 | AVGLPVDKNIPVIGFIGRLEEQKGSDILVAAISKF-VG-LDVQIIILGTGKKKFEQQIQE | 447 |
| 819 | 228210 | 389 | AVGLPVDKKIPLIGFIGRLEEQKGSDILVAAIHKF-IG-LDVQIVVLGTGKKEFEQEIEQ | 446 |
| 820 | 297196 | 389 | AVGLPVDKKIPLIGFIGRLEEQKGSDILVAAIHKF-IG-LDVQIVVLGTGKKEFEQEIEQ | 446 |
| 821 | 2833388 | 390 | EVGLPVDRNVPLIGFIGRLEEQKGSDIFVAAISQL-VE-HNVQIVILGTGKKKFEKQIEH | 447 |
| 822 | 15637079 | 390 | AVGLPVDRNIPLIGFIGRLEEQKGSDILYAAISKF-IS-MDVQILILGTGKKKFEQQIEQ | 447 |
| 823 | 602594 | 389 | AVGLPVDKKVPLIGFIGRLEEQKGSDILVAAIHKF-IG-LDVQIVVLGTGKKEFEQEIEQ | 446 |
| 824 | 2833381 | 390 | AVGLPVDRNIPLIGFIGRLEEQKGSDILYAAISKF-IS-MDVQILILGTGKKKFEQQIEQ | 447 |
| 825 | 5441242 | 388 | EVGLPVNRDIPLIGFIGRLEEQKGSDILVEAIPKF-ID-QNVQIIILGTGKKSMEKQIEQ | 445 |
| 826 | 15223331 | 392 | AVGLPVDRDVPVIGFIGRLEEQKGSDILVEAISKF-MG-LNVQMVILGTGKKKMEAQILE | 449 |
| 827 | 12003285 | 386 | EVGLPVDRNVPLVGFIGRLEEQKGSDILVAALHKF-IE-MDVQVVILGTGKKEFEKQIEQ | 443 |
| 828 | 3832512 | 389 | EVGLPVDRNIPVIGFIGRLEEQKGSDILVESIPKF-ID-QNVQIIVLGTGKKIMEKQIEQ | 446 |
| 829 | 15626365 | 395 | EVGLPVDKNVPLIAFIGRLEEQKGSDILVEAIPQF-IK-ENVQIVALGTGKKEMEKQLQQ | 452 |
| 830 | 2833383 | 385 | EIGLPVDSSIPLIGFIGRLEEQKGSDILVEAIAKF-AD-ENVQIVVLGTGKKIMEKQIEV | 442 |
| 831 | 6624281 | 385 | EVGLPVDRKVPLVAFIGRLEEQKGPDVMIAAIPEI-VKEEDVQIVLLGTGKKKFERLLKS | 443 |
| 832 | 17736918 | 315 | EVGLPVDRKVPLVAFIGRLEEQKGPDVMIAAIPEILKE-EDVQIVLLGTGKKKFERLLKS | 373 |
| 833 | 4760584 | 385 | EVGLPVDRKVPLVAFIGRLEEQKGPDVMIAAIPEILKE-EDVQIVLLGTGKKKFERLLKS | 443 |
| 834 | 6492245 | 381 | EVGLPVDSSIPVIVFIGRLEEQKGSDILIAAIPEF-LE-ENVQIIVLGTGKKKMEEELML | 438 |
| 784 | MaizeSSIIb | 561 | FESEHSDKVRAWVGFSVPLAHRITAGADILLMPSRFEPCGLNQLYAMAYGTVPVVHAVGG | 620 |
| 785 | 7489711 | 561 | FESEHSDKVRAWVGFSVPLAHRITAGADILLMPSRFEPCGLNQLYAMAYGTVPVVHAVGG | 620 |
| 786 | 15028467 | 557 | FESEHNDKVRGWVGFSVQLAHRITAGADVLLMPSRFEPCGLNQLYAMAYSTVPVVHAVGG | 616 |
| 787 | 15384987 | 500 | FESEHNDKVRGWVGFSVQLAHRITAGADVLLMPSRFEPCGLNQLYAMAYGTVPVVHAVGG | 559 |
| 788 | 7489710 | 595 | LEREHPNKVRGWVGFSVPMAHRITAGADVLVMPSRFEPCGLNQLYAMAYGTVPVVHAVGG | 654 |
| 789 | 16265834 | 673 | FEAQHNSKVRGWVGFSVKMAHRITAGADVLVMPSRFEPCGLNQLYAMAYGTVPVVHAVGG | 732 |
| 790 | 15232051 | 655 | MEHQYRDKARGWVGFSVKTAHRITAGADILLMPSRFEPCGLNQLYAMNYGTIPVVHAVGG | 714 |
| 791 | 7489695 | 354 | FEREHHDKVRGWVGFSVRLAHRITAGADALLMPSRFEPCGLNQLYAMAYGTVPVVHAVGG | 413 |
| 792 | 6467503 | 614 | FENQHRDKVRGWVGFSVKTAHRITAGADILLMPSRFEPCGLNQLYAMMYGTIPVVHAVGG | 673 |
| 793 | 2833384 | 615 | FEAQHCDKIRSWVGFSVKMAHRITAGSDILLMPSRFEPCGLNQLYAMSYGTVPVVHGVGG | 674 |
| 794 | 2129898 | 615 | FEAQHCDKIRSWVGFSVKMAHRITAGSDILLMPSRFEPCGLNQLYAMSYGTVPVVHGVGG | 674 |
| 795 | 3192881 | 493 | IEGQYGDKVRGWVGFSVKTAHRITAGALILLMPSRFEPCGLNQLYAMSYGTVPVVHAVGG | 552 |
| 796 | 8953573 | 661 | FEREHHDKVRGWVGFSVRLAHRITAGADALLMPSRFEPCGLNQLYAMAYGTVPVVHAVGG | 720 |
| 797 | 7529653 | 662 | FEREHHDKVRGWVGFSVRLAHRITAGADALLMPSRFEPCGLNQLYAMAYGTVPVVHAVGG | 721 |
| 798 | 8953571 | 662 | FEREHHDKVRGWVGFSVRLAHRITAGADALLMPSRFEPCGLNQLYAMAYGTVPVVHAVGG | 721 |
| 799 | 5825480 | 662 | FEREHHDKVRGWVGFSVRLAHRITAGADALLMPSRFEPCGLNQLYAMAYGTVPVVHAVGG | 721 |
| 800 | 14495348 | 612 | LESQHYDRVRGWVGFSIRLAHRMTAGADILLMPSRFEPCGLNQLYAMMYGTVPVVHAVGG | 671 |
| 801 | 2833390 | 651 | FECQHNDKIRGWVGFSVKTSHRITAGADILLMPSRFEPCALNQLYAMKYGTIPVVHAVGG | 710 |
| 802 | 8708896 | 439 | MENRNKNQCRGWVGFSNKMAHRITAAADILLMPSRFEPCGLNQLYAMAYGTVPIVHSVGG | 498 |
| 803 | 5880466 | 503 | TESSYKDKFRGWVGFSVPVSHRITAGCDILLMPSRFEPCGLNQLYAMQYGTVPVVHGTGG | 562 |
| 804 | 9369334 | 503 | TESSYKDKFRGWVGFSVPVSHRITAGCDILLMPSRFEPCGLNQLYAMQYGTVPVVHGTGG | 562 |
| 805 | 15237934 | 507 | MEETYRDKFRGWVGFNVPISHRITAGCDILLMPSRFEPCGLNQLYAMRYGTIPVVHGTGG | 566 |
| 806 | 7188796 | 499 | TESSYKDKFRGWVGFSVPVSHRITAGCDILLMPSRFEPCGLNQLYAMQYGTVPVVHGTGG | 558 |
| 807 | 6103327 | 503 | TESSYKDKFRGWVGFSVPVSHRITAGCDILLMPSRFEPCGLNQLYAMQYGTVPVVHGTGG | 562 |
| 808 | 9369336 | 503 | TESSYKDKFRGWVGFSVPVSHRITAGCDILLMPSRFEPCGLNQLYAMQYGTVPVVHGTGG | 562 |
| 809 | 6690399 | 430 | MEETYRDKFRGWVGFNVPISHRITAGCDILLMPSRFEPCGLNQLYAMRYGTIPVVHGTGG | 489 |
| 810 | 2829792 | 496 | TENLFKDKFRAWVGFNVPVSHRITAGCDILLMPSRFEPCGLNQLYAMRYGTIPIVHSTGG | 555 |
| 811 | 2833387 | 357 | TESSYKDKFRGWVGFSVPVSHRITAGCDILLMPSRFEPCGLNQLYAMQYGTVPVVHGTGG | 416 |
| 812 | 12019656 | 503 | TESDFKDKFRGWVGFSVPVSHRITAGCDILLMPSRFEPCGLNQLYAMQYGTVPVVHATGG | 562 |
| 813 | 7489712 | 496 | TESIFKDKFRGWVGFSVPVSHRITAGCDILLMPSRFEPCGLNQLYAMQYGTVPVVHATGG | 555 |
| 814 | 7484400 | 185 | MENRNKNQCRGWVGFSNKMAHRITAAADILLMPSRFEPCGLNQLYAMAYGTVPIVHSVGG | 244 |
| 815 | 5295947 | 497 | TESGYRDKFRGWVGFSVPVSHRITAGCDILLMPSRFEPCGLNQLYAMQYGTVPVVHGTGG | 556 |
| 816 | 1549232 | 497 | TESGYRDKFRGWVGFSVPVSHRITAGCDILLMPSRFEPCGLNQLYAMQYGTVPVVHGTGG | 556 |
| 817 | 2833377 | 497 | TESGYRDKFRGWVGFSVPVSHRITAGCDILLMPSRFEPCGLNQLYAMQYGTVPVVHGTGG | 556 |
| 818 | 6136121 | 448 | LEVLYPDKARGVAKFNVPLAHMITAGADFMLVPSRFEPCGLIQLHAMRYGTIPICASTGG | 507 |
| 819 | 228210 | 447 | LEVLYPGKVKGVAKFNVPLAHMITAGADFMLVPSRFEPCGLIQLHAMRYGTVPICASTGG | 506 |
| 820 | 297196 | 447 | LEVLYPNKAKGVAKFNVPLAHMITAGADFMLVPSRFEPCGLIQLHAMRYGTVPICASTGG | 506 |
| 821 | 2833388 | 448 | LEVLYPDKARGVAKFNVPLAHMITAGADFMLVPSRFEPCGLIQLHAMRYGTVPIVASTGG | 507 |
| 822 | 15637079 | 448 | LEVMYPDKARGVAKFNVPLAHMITAGADFMLIPSRFEPCGLIQLHAMRYGTPCICASTGG | 507 |
| 823 | 602594 | 447 | LEVLYPNKAKGVAKFNVPLAHMITAGADFMLVPSRFEPCGLIQLHAMRYGTVPICASTGG | 506 |
| 824 | 2833381 | 448 | LEVMYPDKARGVAKFNVPLAHMITAGADFMLIPSRFEPCGLIQLHAMRYGTPCICASTGG | 507 |
| 825 | 5441242 | 446 | LEEIYPEKARGIAKFDGPLAHKIIAGSDFIMIPSRFEPCGLVQLHSMPYGTVPIVSSTGG | 505 |
| 826 | 15223331 | 450 | LEEKFPGKAVGVAKFNVPLATMITAGADFIIVPSRFEPCGLIQLHAMRYGTVPIVASTGG | 509 |
| 827 | 12003285 | 444 | LEELYPGKAVGVAKFNVPLAHKITAGADFMLVPSRFEPCGLIQLHAMRYGTIPICASTGG | 503 |
| 828 | 3832512 | 447 | LEVTYPGKAIGVAKFNSPLAHKIIAGADFIVIPSRFEPCGLVQLHAMPYGTVPIVSSTGG | 506 |
| 829 | 15626365 | 453 | LEISYPDKARGVAKFNVPLAHMMIAGADFILIPSRFEPCGLIQLQAMRYGTVPIVASTGG | 512 |
| 830 | 2833383 | 443 | LEEKYPGKAIGITKFNSPLAHKIIAGADFIVIPSRFEPCGLVQLHAMPYGTVPIVSSTGG | 502 |
| 831 | 6624281 | 444 | VEEKFPTKVRAVVRFNAPLAHQMMAGADVLAVTSRFEPCGLIQLQGMRYGTPCACASTGG | 503 |
| 832 | 17736918 | 374 | IEEKFPSKVRAVVRFNAPLAHQMMAGADVLAVTSRFEPCGLIQLQGMRYGTPCACASTGG | 433 |
| 833 | 4760584 | 444 | IEEKFPSKVRAVVRFNAPLAHQMMAGADVLAVTSRFEPCGLIQLQGMRYGTPCACASTGG | 503 |
| 834 | 6492245 | 439 | LEAKYPQNARGIAKFNVPLAHMMFAGANFIIVPSRFEPCGLIQLQGMRYGVIPICSSTGG | 498 |
| 784 | MaizeSSIIb | 621 | LRDTV---------AP-FDPF----NDT---GL----G---W--------TFDR---AEA | 645 |
| 785 | 7489711 | 621 | LRDTV---------AP-FDPF----NDT---GL----G---W--------TFDR---AEA | 645 |
| 786 | 15028467 | 617 | LRDTV---------AP-FDPF----ADT---GL----G---W--------TFDR---AEA | 641 |
| 787 | 15384987 | 560 | LRDTV---------AP-FDPF----ADT---GL----G---W--------TFDR---AEA | 584 |
| 788 | 7489710 | 655 | LRDTV---------AP-FDPF----GDA---GL----G---W--------TFDR---AEA | 679 |
| 789 | 16265834 | 733 | LRDTM---------SA-FDPF----EDT---GL----G---W--------TFDR---AEP | 757 |
| 790 | 15232051 | 715 | LRDTV---------QQ-FDPY----SET---GL----G---W--------TFDS---AEA | 739 |
| 791 | 7489695 | 414 | LRDTV---------PP-FDPF----NHS---GL----G---W--------TFDR---AEA | 438 |
| 792 | 6467503 | 674 | LRDTV---------QP-FDPF----NES---GL----G---W--------TFDS---AES | 698 |
| 793 | 2833384 | 675 | LRDTV---------QP-FNPF----DES---GV----G---W--------TFDR---AEA | 699 |
| 794 | 2129898 | 675 | LRDTV---------QP-FNPF----DES---GV----G---W--------TFDR---AEA | 699 |
| 795 | 3192881 | 553 | LRDTV---------QP-FDPF----NES---GY----G---W--------TFGR---AEA | 577 |
| 796 | 8953573 | 721 | LRDTV---------PP-FDPF----NHS---GL----G---W--------TFDR---AEA | 745 |
| 797 | 7529653 | 722 | LRDTV---------PP-FDPF----NHS---GL----G---W--------TFDR---AEA | 746 |
| 798 | 8953571 | 722 | LRDTV---------PP-FDPF----NHS---GL----G---W--------TFDR---AEA | 746 |
| 799 | 5825480 | 722 | VRDTV---------PP-FDPF----NHS---GL----G---W--------TFDR---AEA | 746 |
| 800 | 14495348 | 672 | LRDTV---------EH-YNPY----EES---GL----G---W--------TFEK---AEA | 696 |
| 801 | 2833390 | 711 | LRDTV---------QP-FDPL----MSQ---DW----G---G--------PSDR---AEA | 735 |
| 802 | 8708896 | 499 | LRDTV---------KQ-YSPF----ENV---GT----G---W--------VFER---AEA | 523 |
| 803 | 5880466 | 563 | LRDTV---------ET-FNPFGAK-GEE---GT----G---W--------AFSP---LTV | 590 |
| 804 | 9369334 | 563 | LRDTV---------ET-FNPFGAK-GEE---GT----G---W--------AFSF---LTV | 590 |
| 805 | 15237934 | 567 | LRDTV---------EN-FNPYAEGGAGT---GT----G---W--------VFTP---LSK | 595 |
| 806 | 7188796 | 559 | LRDTV---------ET-FNPFGAK-GEE---GT----G---W--------AFSP---LTV | 586 |
| 807 | 6103327 | 563 | LRDTV---------ET-FNPFGAK-GEE---GT----G---W--------AFSP---LTV | 590 |
| 808 | 9369336 | 563 | LRDTV---------ET-FNPFGAK-GEE---GT----G---W--------AFSP---LTV | 590 |
| 809 | 6690399 | 490 | LRDTV---------EN-FNPYAEGGAGA---GT----G---W--------VFTP---LSK | 518 |
| 810 | 2829792 | 556 | LRDTV---------KD-FNPY----AQE---GIGEGTG---W--------TFSP---LTS | 584 |
| 811 | 2833387 | 417 | LRDTV---------ET-FNPFGAK-GEE---GT----G---W--------AFSP---LTV | 444 |
| 812 | 12019656 | 563 | LRDTV---------EN-FNPF----GENGEQGT----G---W--------AFAP---LTT | 590 |
| 813 | 7489712 | 556 | LRDTV---------EN-FNPF----GENGEQGT----G---W--------AFAP---LTT | 583 |
| 814 | 7484400 | 245 | LRDTV---------KQ-YSPF----ENV---GT----G---W--------VFER---AEA | 269 |
| 815 | 5295947 | 557 | LRDTV---------EN-FNPFAEK-GEQ---GT----G---W--------AF | 579 |
| 816 | 1549232 | 557 | LRDTV---------EN-FNPFAEK-GEQ---GT----G---W--------AF | 579 |
| 817 | 2833377 | 557 | LRDTV---------EN-FNPFAEK-GEQ---GT----G---W--------AF | 579 |
| 818 | 6136121 | 508 | LVDTV---------TEGFTGF----HMG---AF----NVECA--------TVDP---ADV | 536 |
| 819 | 228210 | 507 | LVDTV---------K---EGY----TGF---HM----G---AFNVECD--VVDP---ADV | 535 |
| 820 | 297196 | 507 | LVDTV---------K---EGY----TGF---HM----G---AFNVECD--VVDP---ADV | 535 |
| 821 | 2833388 | 508 | LVDTV---------KEGYTGF----QMG---AL----H---V--------ECDKIDSADV | 536 |
| 822 | 15637079 | 508 | LVDTV---------K---EGY----TGF---HM----G---AFNVDCE--TVDP---EDV | 536 |
| 823 | 602594 | 507 | LVDTV---------K---EGY----TGF---HM----G---AFNVECD--VVDP---ADV | 535 |
| 824 | 2833381 | 508 | LVDTV---------K---EGY----TGF---HM----G---AFNVDCE--TVDP---EDV | 536 |
| 825 | 5441242 | 506 | LVDTV---------Q---EGF----TGF---HM----G---AFNVDCE--AIDP---ADV | 534 |
| 826 | 15223331 | 510 | LVDTV---------K---DGY----TGF---HI----G---RFNVKCE--VVDP---DDV | 538 |
| 827 | 12003285 | 504 | LVDTV---------K---EGF----TGF---HM----G---AFNVECD--AVDP---ADV | 532 |
| 828 | 3832512 | 507 | LVDTV---------KEGYTGF----HVG---AF----SVECE--------AVDP---ADV | 535 |
| 829 | 15626365 | 513 | LVDTV---------K---EGF----TGF---HM----G---SFNVKCD--AVDP---VDV | 541 |
| 830 | 2833383 | 503 | LVDTVKEGYTGFHAGP-FDVE----CE-----------------------DVDP---DDV | 531 |
| 831 | 6824281 | 504 | LVDTI---------VE---------GKT---GF----H---MGRLSVDCNVVEP---ADV | 532 |
| 832 | 17736918 | 434 | LVDTI---------VE---------GKT---GF----H---MGRLSVDCNVVEP---ADV | 462 |
| 833 | 4760584 | 504 | LVDTI---------VE---------GKT---GF----H---MGRLSVDCNVVEP---ADV | 532 |
| 834 | 6492245 | 499 | LVDTV---------SEGVTGF----HMG---SF----N---V--------EFET---VDP | 524 |

TABLE XXIX
Maize soluble starch synthase IIb (SSIIb)
“CTEND” Domain Alignments with other similar proteins

| SEQ | Accession | a.a | | a.a. |
|---|---|---|---|---|
| Id.No. | Number | # | (start) Sequence end | # |
| 835 | MaizeSSIIb | 646 | NRMIDALSHCLTTY--RN--YKE-SWRACRARGMAEDLSWDHAAVLYEDVLVKAKYQW | 698 |
| 836 | 7489711 | 646 | NRMIDALSHCLTTY--RN--YKE-SWRACRARGMAEDLSNDHAAVLYEDVLVKAKYQW | 698 |
| 837 | 15028467 | 642 | NRMIDALGHCLNTY--RN--YKE-SWRGLQARGMAQDLSWDRAAELYEDVLVKAKYQW | 694 |
| 838 | 15384987 | 585 | NRMIDALGHCLNTY--RN--YKE-SWRGLQARGMAQDLSWDHAAELYEDVLVKAKYQW | 637 |
| 839 | 7489710 | 680 | NKLIEALRHCLDTY--RK--YGE-SWKSLQARGMSQDLSWDHAAELYEDVLVKAKYQW | 732 |
| 840 | 16265834 | 758 | HKLIEALGHCLETY--RK--YKE-SWRGLQVRGMSQDLSWDHAAELYEEVLVKAKYQW | 810 |
| 841 | 15232051 | 740 | GKLIHALGNCLLTY--RE--YKE-SWEGLQRRGMTQDLSWDNAAEKYEEVLVAAKYHW | 792 |
| 842 | 7489695 | 439 | HKLIEALGHCLRTY--RD--FKE-SWRALQERGMSQDFSWEHAAKLYEDVLVKAKYQW | 491 |
| 843 | 6467503 | 699 | HKLIHALGNCLLTY--RE--YKK-SWEGLQRRGMTPNLSWDHAAEKYEETLVAAKYQW | 751 |
| 844 | 2833384 | 700 | NKLMAALWNCLLTY--KD--YKK-SWEGIQERGMSQDLSWDNAAQQYEEVLVAAKYQW | 752 |
| 845 | 2129898 | 700 | NKLMAALWNCLLTY--KD--YKK-SWEGIQERGMSQDLSWDNAAQQYEEVLVAAKYQW | 752 |
| 846 | 3192881 | 578 | NQLIDALGNCLLTY--RQ--YKQ-SWEGLQRRGMMQDLSWDHAAEKYEEVLVAAKYQW | 630 |
| 847 | 8953573 | 746 | QKLIEALGHCLRTY--RD--YKE-SWRGLQERGMSQDFSWEHAAKLYEDVLVKAKYQW | 798 |
| 848 | 7529653 | 747 | HKLIEALGHCLRTY--RD--FKE-SWRALQERGMSQDFSWEHAAKLYEDVLVKAKYQW | 799 |
| 849 | 8953571 | 747 | HKLIEALGHCLRTY--RD--YKE-SWRGLQERGMSQDFSWEHAAKLYEDVLLKAKYQW | 799 |
| 850 | 5825480 | 747 | HKLIEALGHCLRTY--RD--YKE-SWRGLQERGMSQDFSWEHAAKLYEDVLLKAKYQW | 799 |
| 851 | 14495348 | 697 | NRLIDALGHCLNTY--RN--YRT-SWEGLQKRGMMQDLSWDNAAKLYEEVLLAAKYQW | 749 |
| 852 | 2833390 | 736 | SQLIPRIRNCLLTY--RE--YKK-SWEGIQTRCMTQDLSWDNAAQNYEEVLIAAKYQW | 788 |
| 853 | 8708896 | 524 | NKLRESINNALYTY--RQ--FRD-SFRGIQRRGMEQDLTWDNAASIYEEVLVAAKYQW | 576 |
| 854 | 5880466 | 591 | DKMLWALRTAMSTF--RE--HKP-SWEGLMKRGMTKDHTWDHAAEQYEQI | 635 |
| 855 | 9369334 | 591 | DKMLWALRTAMSTF--RE--HKP-SWEGLMKRGMTKDHTWDHAAEQYEQI | 635 |
| 856 | 15237934 | 596 | DSMVSALRLAAATY--RE--YKQ-SWEGLMRRGMTRNYSWENAAVQYEQV-----FQW | 643 |
| 857 | 7188796 | 587 | EKMLWALRTAISTF--RE--HKP-SWEGLMKRGMTKDHTWDHAAEQYEQI | 631 |
| 858 | 6103327 | 591 | DKMLWALRTAMSTF--RE--HKP-SWEGLMKRGMTKDHTWDHAAEQYEQI | 635 |
| 859 | 9369336 | 591 | DKMLWALRTANSTF--RE--HKP-SWEGLMKRGMTKDHTWDRAAEQYEQI | 635 |
| 860 | 6690399 | 519 | DSMVSALRLAAATY--RE--YKQ-SWEGLMRRGMTRNYSWENAAVQYEQV-----FQW | 566 |
| 861 | 2829792 | 585 | EKLLDTLKLAIGTY--TE--HKS-SWEGLMRRGMGRDYSWENAAIQYEQVFTWA | 633 |
| 862 | 2833387 | 445 | DKMLWALRTAMSTF--RE--HKP-SWEGLMKRGMTKDHTWDHA | 482 |
| 863 | 12019656 | 591 | ENMFVDIANC | 600 |
| 864 | 7489712 | 584 | ENMFVDIANC | 593 |
| 865 | 7484400 | 270 | NKLRESINNALYTY--RQ--FRD-SFRGIQRRGMEQDLTWDNAASIYEEVLVAAKYQW | 322 |
| 866 | 6136121 | 537 | QKIATTVERALAAY--GSVAYKE-MIQNC----MAQDLSWKGPAKNWEKMLL | 581 |
| 867 | 228210 | 536 | LKIVTTVAPALAVY--GTLAFAE-MIKNC----MSEELSWKEPAKKWETLLL | 580 |
| 868 | 267196 | 536 | LKIVTTVARALAVY--GTLAFAE-MIKNC----MSEELSWKEPAKKWETLLL | 580 |
| 869 | 2833388 | 537 | AAIVKTVAPALGTY--AT----A-ALREMILNCMAQDLSWKGPARMWEKMLL | 581 |
| 870 | 15637079 | 537 | LKVITTVGRALAMY--GTLAFTE-MIKNC----MSQELSWKGPAKNWETVLL | 581 |
| 871 | 602594 | 536 | LKIVTTVARALAVY--GTLAFAE-MIKNC----MSEELSWKEPAKKWETLLL | 580 |
| 872 | 2833381 | 537 | LKVITTVGRALAIY--GTLAFTE-MIKNC----MSQELSWKGPAKNWETVLL | 581 |
| 873 | 5441242 | 535 | EKIATTVRRALGTY--GT--V---AMEKIIQNCMAQDFSWKGPAKQWEKVL | 578 |
| 874 | 15223331 | 539 | IATAKPNTRAVAVYGTSA--MQE-MVKNC----MDQDFSWKGPARLWEKVLL | 583 |
| 875 | 12003285 | 533 | LKIVKTVGRALEVY--GTPAFRE-MINNC----MSLDLSWKGPAKNWETVLL | 577 |
| 876 | 3832512 | 536 | EKLATTVNRALKTYGTQA--LKE-MILNC----MAQDFSWKGPAKQWEQALL | 580 |
| 877 | 15626365 | 542 | DAIPKTVTKALGVYGTSA--FAE-MIKNC----MAQELSWKGPAKKWEEVLL | 586 |
| 878 | 2833383 | 532 | DKLAATVKRALKTY--GT----Q-AMKQIILNCMAQNFSWKKPAKLWEKALL | 576 |
| 879 | 6624281 | 533 | KKVVTTLKRAVKVV--GTPAYHE-MVKNC----MIQDLSWKGPAKNWEDVLLE | 578 |
| 880 | 17736918 | 463 | KKVVTTLKRAVKVV--GTPAYHE-MVKNC----MIQDLSWKGPAKNWEDVLLE | 508 |
| 881 | 4760584 | 533 | KKVVTTLKRAVKVV--GTPAYHE-MVKNC----MIQDLSWKGPAKNWEDVLLE | 578 |
| 882 | 6492245 | 525 | ADVAAVASNVTRAL--KQ--YKTPSFHAMVQNCMAQDLSWKGPAKKWEEALL | 572 |

TABLE XXX
Identities of the Accession Numbers used in Tables. XXV-XXIX.

| Accession | Brief Description of sequences | score | |
|---|---|---|---|
| Id. | producing significant alignments | (bits) | E−value |
| gi\|7489711\|pir\|\|T01209 | ADPglucose - starch glucosyltransfera . . . | 1240 | 0.0 h |
| gi\|15028467\|gb\|AAK81729.1\|AF395537_1 | (AF395537) soluble sta . . . | 972 | 0.0 |
| gi\|15384987\|emb\|CAC59826.1\| | (AJ308110) soluble starch synth . . . | 968 | 0.0 |
| gi\|7489710\|pir\|\|T01208 | ADPglucose - starch glucosyltransfera . . . | 869 | 0.0 |
| gi\|16265834\|gb\|AAL16661.1\|AF419099_1 | (AF419099) putative so . . . | 847 | 0.0 |
| gi\|15232051\|ref\|NP_186767.1\| | (NM_110984) putative glycogen . . . | 837 | 0.0 |
| gi\|7489695\|pir\|\|T06798 | probable starch synthase (EC 2.4.1.— . . . | 833 | 0.0 |
| gi\|6467503\|gb\|AAF13168.1\|AF173900_1 | (AF173900) granule boun . . . | 832 | 0.0 |
| gi\|2833384\|sp\|Q43093\|UGS3_PEA | Glycogen [starch] synthase, c . . . | 832 | 0.0 |
| gi\|2129898\|pir\|\|S61505 | UDPglucose - starch glucosyltransfera . . . | 831 | 0.0 |
| gi\|3192881\|gb\|AAC19119.1\| | (AF068834) starch synthase [Ipomo . . . | 823 | 0.0 |
| gi\|8953573\|emb\|CAB96627.1\| | (AJ269504) starch synthase IIa-3 . . . | 814 | 0.0 |
| gi\|7529653\|emb\|CAB86618.1\| | (AJ269502) starch synthase IIa-1 . . . | 812 | 0.0 |
| gi\|8953571\|emb\|CAB96626.1\| | (AJ269503) starch synthase IIa-2 . . . | 811 | 0.0 |
| gi\|5825480\|gb\|AAD53263.1\|AF155217_1 | (AF155217) starch synth . . . | 809 | 0.0 |
| gi\|14495348\|gb\|AAK64284.1\|AF383878_1 | (AF383878) soluble sta . . . | 802 | 0.0 |
| gi\|2833390\|sp\|Q43847\|UGS3_SOLTU | Glycogen [starch] synthase, . . . | 785 | 0.0 |
| gi\|8708896\|gb\|AAC17970.2\| | (AF026421) soluble starch synthas . . . | 669 | 0.0 |
| gi\|5880466\|gb\|AAD54661.1\| | (AF091803) starch synthase I [Tri . . . | 456 | e−127 |
| gi\|9369334\|emb\|CAB99209.1\| | (AJ292521) starch synthase I-1 [. . . | 456 | e−127 |
| gi\|15237934\|ref\|NP_197818.1\| | (NM_122336) soluble starch syn . . . | 455 | e−127 |
| gi\|7188796\|gb\|AAF37876.1\|AF234163_1 | (AF234163) starch synth . . . | 454 | e−126 |
| gi\|6103327\|gb\|AAF03557.1\| | (AF091802) starch synthase I [Aeg . . . | 454 | e−126 |
| gi\|9369336\|emb\|CAB99210.1\| | (AJ292522) starch synthase I-2 [. . . | 454 | e−126 |
| gi\|6690399\|gb\|AAF24126.1\|AF121673_1 | (AF121673) soluble star . . . | 453 | e−126 |
| gi\|2829792\|sp\|P93568\|UGS2_SOLTU | Soluble glycogen [starch] s . . . | 449 | e−125 |
| gi\|2833387\|sp\|Q43654\|UGS2_WHEAT | Soluble glycogen [starch] s . . . | 448 | e−125 |
| gi\|12019656\|gb\|AAD45815.2\| | (AF168786) soluble starch syntha . . . | 423 | e−117 |
| gi\|7489712\|pir\|\|T01414 | ADPglucose - starch glucosyltransfera . . . | 422 | e−117 |
| gi\|7484400\|pir\|\|T07924 | probable starch synthase (EC 2.4.1.— . . . | 413 | e−114 |
| gi\|5295947\|dbj\|BAA81848.1\| | (AB026295) ESTs AU075322(C11109) . . . | 412 | e−114 |
| gi\|1549232\|dbj\|BAA07396.1\| | (D38221) SSS1 [Oryza sativa] >gi . . . | 412 | e−114 |
| gi\|2833377\|sp\|Q40739\|UGS2_QRYSA | Soluble glycogen [starch] s . . . | 411 | e−113 |
| gi\|6136121\|sp\|O82627\|UGST_ANTMA | Granule-bound glycogen [sta . . . | 346 | 4e−94 |
| gi\|228210\|prf\|\|1718316A | granule-bound starch synthase [Sola . . . | 327 | 2e−88 |
| gi\|267196\|sp\|Q00775\|UGST_SOLTU | Granule-bound glycogen [star . . . | 327 | 3e−88 |
| gi\|2833388\|sp\|Q43784\|UGST_MANES | Granule-bound glycogen [sta . . . | 327 | 3e−88 |
| gi\|15637079\|dbj\|BAB68126.1\| | (AB071604) granule-bound starch . . . | 326 | 5e−88 |
| gi\|602594\|emb\|CAA58220.1\| | (X83220) starch (bacterial glycog . . . | 326 | 6e−88 |
| gi\|2833381\|sp\|Q42857\|UGST_IPOBA | Granule-bound glycogen [sta . . . | 320 | 2e−86 |
| gi\|5441242\|dbj\|BAA82346.1\| | (AB029546) granule-bound starch . . . | 318 | 9e−86 |
| gi\|15223331\|ref\|NP_174566.1\| | (NM_103023) starch synthase, p . . . | 318 | 1e−85 |
| gi\|12003285\|gb\|AAG43519.1\|AF210699_1 | (AF210699) granule-bou . . . | 316 | 6e−85 |
| gi\|3832512\|gb\|AAC70779.1\| | (AF097922) granule-bound glycogen . . . | 314 | 2e−84 |
| gi\|15626365\|emb\|CAC69955.1\| | (AJ345045) granule-bound starch . . . | 313 | 3e−84 |
| gi\|2833383\|sp\|Q43092\|UGST_PEA | Granule-bound glycogen [starc . . . | 312 | 1e−83 |
| gi\|6624281\|dbj\|BAA88509.1\| | (AB029061) starch synthase (GBSS . . . | 311 | 1e−83 |
| gi\|17736918\|gb\|AAL41028.1\| | (AF250137) mutant granule bound . . . | 311 | 2e−83 |
| gi\|4760584\|dbj\|BAA77352.1\| | (AB019624) starch synthase (GBSS . . . | 311 | 2e−83 |
| gi\|6492245\|gb\|AAF14233.1\|AF109395_1 | (AF109395) granule-boun . . . | 309 | 5e−83 |
| gi\|6624285\|dbj\|BAA88511.1\| | (AB029063) starch synthase (GBSS . . . | 308 | 1e−82 |
| gi\|11037536\|gb\|AAG27624.1\|AF286320_1 | (AF286320) granule bou . . . | 308 | 1e−82 |
| gi\|4760582\|dbj\|BAA77351.1\| | (AB019623) starch synthase (GBSS . . . | 308 | 1e−82 |
| gi\|6318540\|gb\|AAF06937.1\|AF110374_1 | (AF110374) granule-boun . . . | 308 | 1e−82 |
| gi\|6318538\|gb\|AAF06936.1\|AF110373_1 | (AF110373) granule-boun . . . | 307 | 2e−82 |
| gi\|6624287\|dbj\|BAA88512.1\| | (AB029064) starch synthase (GBSS . . . | 306 | 3e−82 |
| gi\|18652407\|gb\|AAL77109.1\|AF474373_6 | (AF474373) granule-bou . . . | 306 | 3e−82 |
| gi\|136755\|sp\|P09842\|UGST_HORVU | Granule-bound glycogen [star . . . | 306 | 4e−82 |
| gi\|18139611\|gb\|AAL58572.1\| | (AY069940) granule binding starc . . . | 305 | 8e−82 |
| gi\|4760580\|dbj\|BAA77350.1\| | (AB019622) starch synthase (GBSS . . . | 305 | 1e−81 |
| gi\|4588609\|gb\|AAD26156.1\|AF113844_1 | (AF113844) granule-boun . . . | 305 | 2e−81 |
| gi\|136765\|sp\|P27736\|UGST_WHEAT | Granule-bound glycogen [star . . . | 304 | 2e−81 |
| gi\|6624283\|dbj\|BAA88510.1\| | (AB029062) starch synthase (GBSS . . . | 303 | 3e−81 |
| gi\|16716335\|gb\|AAC17969.3\| | (AF026420) granule-bound starch . . . | 300 | 3e−80 |
| gi\|82478\|pir\|\|JQ0703 | UDPglucose - starch glucosyltransferase . . . | 299 | 6e−80 |
| gi\|2833385\|sp\|Q43134\|UGST_SORBI | Granule-bound glycogen [sta . . . | 298 | 1e−79 |
| gi\|15900991\|ref\|NP_345595.1\| | (NC_003028) glycogen synthase . . . | 298 | 1e−79 |
| gi\|15903076\|ref\|NP_358626.1\| | (NC_003098) Glycogen synthase . . . | 298 | 2e−79 |
| gi\|2833382\|sp\|Q42968\|UGST_ORYGL | Granule-bound glycogen [sta . . . | 297 | 2e−79 |
| gi\|297424\|emb\|CAA46294.1\| | (X65183) glycogen (starch) syntha . . . | 296 | 3e−79 |
| gi\|136758\|sp\|P19395\|UGST_ORYSA | Granule-bound glycogen [star . . . | 296 | 5e−79 |
| gi\|7798551\|gb\|AAC61675.2\| | (AF031162) granule-bound starch s . . . | 296 | 5e−79 |
| gi\|297422\|emb\|CAA45472.1\| | (X64108) starch granule-bound sta . . . | 292 | 1e−77 |
| gi\|4588607\|gb\|AAD26155.1\|AF113843_1 | (AF113843) granule-boun . . . | 289 | 9e−77 |
| gi\|136757\|sp\|P04713\|UGST_MAIZE | Granule-bound glycogen [star . . . | 288 | 1e−76 |
| gi\|15643657\|ref\|NP_228703.1\| | (NC_000853) glycogen synthase . . . | 285 | 9e−76 |
| gi\|16080147\|ref\|NP_390973.1\| | (NC_000964) starch (bacterial . . . | 276 | 7e−73 |
| gi\|15672681\|ref\|NP_266855.1\| | (NC_002662) glycogen synthase . . . | 273 | 6e−72 |
| gi\|17366711\|sp\|Q9CHM9\|GLGA_LACLA | Glycogen synthase (Starch . . . | 273 | 6e−72 |
| gi\|2811062\|sp\|O08328\|GLGA_BACST | Glycogen synthase (Starch [. . . | 267 | 2e−70 |
| gi\|18309046\|ref\|NP_560980.1\| | (NC_003366) glycogen synthase . . . | 264 | 2e−69 |
| gi\|15613648\|ref\|NP_241951.1\| | (NC_002570) starch (bacterial . . . | 258 | 1e−67 |
| gi\|15895507\|ref\|NP_348856.1\| | (NC_003030) Glycogen synthase, . . . | 255 | 1e−66 |
| gi\|17229371\|ref\|NP_485919.1\| | (NC_003272) glycogen synthase . . . | 246 | 6e−64 |
| gi\|15641730\|ref\|NP_231362.1\| | (NC_002505) glycogen synthase . . . | 236 | 6e−61 |
| gi\|15620537\|gb\|AAL03921.1\|U30252_9 | (U30252) GlgA [Synechoco . . . | 235 | 1e−60 |
| gi\|16331219\|ref\|NP_441947.1\| | (NC_000911) glycogen synthase . . . | 235 | 1e−60 |
| gi\|16766821\|ref\|NP_462436.1\| | (NC_003197) glycogen synthase . . . | 229 | 7e−59 |
| gi\|16762766\|ref\|NP_458383.1\| | (NC_003198) glycogen synthase . . . | 228 | 2e−58 |
| gi\|4582783\|emb\|CAB40375.1\| | (AJ006752) starch synthase, isof . . . | 226 | 4e−58 |
| gi\|17938870\|ref\|NP_535658.1\| | (NC_003306) glycogen synthase . . . | 225 | 2e−57 |
| gi\|16119514\|ref\|NP_396220.1\| | (NC_003064) AGR_pAT_410p [Agro . . . | 224 | 2e−57 |
| gi\|16124067\|ref\|NP_407380.1\| | (NC_003143) glycogen synthase . . . | 223 | 6e−57 |
| gi\|15717885\|gb\|AAK97773.1\| | (AY044844) starch synthase isofo . . . | 222 | 1e−56 |
| gi\|15803938\|ref\|NP_289974.1\| | (NC_002655) glycogen synthase . . . | 220 | 3e−56 |
| gi\|15890897\|ref\|NP_356569.1\| | (NC_003063) AGR_L_1562p [Agrob . . . | 219 | 9e−56 |
| gi\|15236819\|ref\|NP_193558.1\| | (NM_117934) starch synthase-li . . . | 216 | 8e−55 |
| gi\|17227527\|ref\|NP_484075.1\| | (NC_003272) glycogen (starch) . . . | 215 | 1e−54 |
| gi\|13476305\|ref\|NP_107875.1\| | (NC_002678) glycogen synthase . . . | 214 | 3e−54 |
| gi\|6116748\|dbj\|BAA85761.1\| | (AB028026) granule-bound starch . . . | 213 | 6e−54 |
| gi\|16329217\|ref\|NP_439945.1\| | (NC_000911) glycogen (starch) . . . | 212 | 1e−53 |
| gi\|9587321\|gb\|AAF89262.1\|AF285987_1 | (AF285987) granule-boun . . . | 211 | 2e−53 |
| gi\|15602409\|ref\|NP_245481.1\| | (NC_002663) GlgA [Pasteurella . . . | 211 | 2e−53 |
| gi\|9587293\|gb\|AAF89248.1\|AF285973_1 | (AF285973) granule-boun . . . | 211 | 2e−53 |
| gi\|14279432\|gb\|AAK58596.1\|AF268969_2 | (AF268969) glycogen sy . . . | 211 | 3e−53 |
| gi\|7489826\|pir\|\|T01265 | starch synthase DULL1 - maize >gi\|30 . . . | 210 | 5e−53 |
| gi\|17366749\|sp\|Q9EUT5\|GLGA_RHITR | Glycogen synthase (Starch . . . | 209 | 6e−53 |
| gi\|4582789\|emb\|CAB40374.1\| | (AJ225088) Starch synthase isofo . . . | 209 | 1e−52 |
| gi\|15966599\|ref\|NP_386952.1\| | (NC_003047) PROBABLE GLYCOGEN . . . | 208 | 2e−52 |
| gi\|9587317\|gb\|AAF89260.1\|AF285985_1 | (AF285985) granule-boun . . . | 207 | 2e−52 |
| gi\|15606118\|ref\|NP_213495.1\| | (NC_000918) glycogen synthase . . . | 207 | 4e−52 |
| gi\|9587301\|gb\|AAF89252.1\|AF285977_1 | (AF285977) granule-boun . . . | 206 | 5e−52 |
| gi\|146139\|gb\|AAA23870.1\| | (J02616) glycogen synthase (EC 2.4 . . . | 206 | 7e−52 |
| gi\|15221083\|ref\|NP_172637.1\| | (NM_101044) putative glycogen . . . | 205 | 2e−51 |
| gi\|18461221\|dbj\|BAB84418.1\| | (AP003292) putative starch synt . . . | 204 | 2e−51 |
| gi\|9587329\|gb\|AAF89266.1\|AF285991_1 | (AF285991) granule-boun . . . | 203 | 6e−51 |
| gi\|16265159\|ref\|NP_437951.1\| | (NC_003078) putative glycogen . . . | 202 | 9e−51 |
| gi\|9587352\|gb\|AAF89276.1\|AF286003_1 | (AF286003) granule-boun . . . | 202 | 1e−50 |
| gi\|7489274\|pir\|\|T07663 | soluble starch synthase (EC 2.4.1.—) . . . | 201 | 3e−50 |
| gi\|9587309\|gb\|AAF89256.1\|AF285981_1 | (AF285981) granule-boun . . . | 200 | 4e−50 |
| gi\|17548463\|ref\|NP_521803.1\| | (NC_003296) PROBABLE GLYCOGEN . . . | 200 | 5e−50 |
| gi\|9587343\|gb\|AAF89273.1\|AF285998_1 | (AF285998) granule-boun . . . | 200 | 5e−50 |
| gi\|9587311\|gb\|AAF89257.1\|AF285982_1 | (AF285982) granule-boun . . . | 199 | 6e−50 |
| gi\|2833389\|sp\|Q43846\|UGS4_SOLTU | Soluble glycogen [starch] s . . . | 199 | 1e−49 |
| gi\|17646328\|gb\|AAL40942.1\|AF432915_1 | (AF432915) putative st . . . | 198 | 1e−49 |
| gi\|9587337\|gb\|AAF89270.1\|AF285995_1 | (AF285995) granule-boun . . . | 198 | 2e−49 |
| gi\|9587341\|gb\|AAF89272.1\|AF285997_1 | (AF285997) granule-boun . . . | 197 | 3e−49 |
| gi\|9587305\|gb\|AAF89254.1\|AF285979_1 | (AF285979) granule-boun . . . | 197 | 3e−49 |
| gi\|16273270\|ref\|NP_439511.1\| | (NC_000907) glycogen synthase . . . | 197 | 4e−49 |
| gi\|9587348\|gb\|AAF89274.1\| | (AF286001) granule-bound starch s . . . | 197 | 4e−49 |
| gi\|9587319\|gb\|AAF89261.1\|AF285986_1 | (AF285986) granule-boun . . . | 196 | 6e−49 |
| gi\|8901183\|gb\|AAC17971.2\| | (AF026422) soluble starch synthas . . . | 195 | 1e−48 |
| gi\|9587307\|gb\|AAF89255.1\|AF285980_1 | (AF285980) granule-boun . . . | 194 | 2e−48 |
| gi\|9587323\|gb\|AAF89263.1\|AF285988_1 | (AF285988) granule-boun . . . | 194 | 2e−48 |
| gi\|9587313\|gb\|AAF89258.1\|AF285983_1 | (AF285983) granule-boun . . . | 193 | 6e−48 |
| gi\|9587299\|gb\|AAF89251.1\|AF285976_1 | (AF285976) granule-boun . . . | 191 | 2e−47 |
| gi\|9587327\|gb\|AAF89265.1\|AF285990_1 | (AF285990) granule-boun . . . | 191 | 2e−47 |
| gi\|9587325\|gb\|AAF89264.1\|AF285989_1 | (AF285989) granule-boun . . . | 190 | 4e−47 |
| gi\|9587297\|gb\|AAF89250.1\|AF285975_1 | (AF285975) granule-boun . . . | 190 | 5e−47 |
| gi\|17367070\|sp\|Q9RNH6\|GLGA_RHOSH | Glycogen synthase (Starch . . . | 190 | 5e−47 |
| gi\|5834407\|gb\|AAD53959.1\|AF181035_3 | (AF181035) glycogen syn . . . | 190 | 5e−47 |
| gi\|9587295\|gb\|AAF89249.1\|AF285974_1 | (AF285974) granule-boun . . . | 188 | 2e−46 |
| gi\|9587333\|gb\|AAF89268.1\|AF285993_1 | (AF285993) granule-boun . . . | 187 | 3e−46 |
| gi\|9502145\|gb\|AAF88000.1\| | (AF258609) starch synthase III [A . . . | 187 | 4e−46 |
| gi\|15597361\|ref\|NP_250855.1\| | (NC_002516) probable glycogen . . . | 186 | 6e−46 |
| gi\|9587339\|gb\|AAF89271.1\|AF285996_1 | (AF285996) granule-boun . . . | 186 | 7e−46 |
| gi\|9502143\|gb\|AAF87999.1\|AF258608_1 | (AF258608) starch synth . . . | 186 | 7e−46 |
| gi\|9587303\|gb\|AAF89253.1\|AF285978_1 | (AF285978) granule-boun . . . | 186 | 7e−46 |
| gi\|9587335\|gb\|AAF89269.1\|AF285994_1 | (AF285994) granule-boun . . . | 185 | 2e−45 |
| gi\|9587331\|gb\|AAF89267.1\|AF285992_1 | (AF285992) granule-boun . . . | 182 | 9e−45 |
| gi\|17366350\|sp\|P58395\|GLGA_THECA | Glycogen synthase (Starch . . . | 180 | 4e−44 |
| gi\|15805621\|ref\|NP_294317.1\| | (NC_001263) glycogen synthase . . . | 180 | 5e−44 |
| gi\|15983795\|gb\|AAL10494.1\| | (AY056803) Atlg32900/F9L11_8 [Ar . . . | 179 | 9e−44 |
| gi\|17367076\|sp\|Q9RWS1\|GLGA_DEIRA | Glycogen synthase (Starch . . . | 177 | 5e−43 |
| gi\|15605532\|ref\|NP_220318.1\| | (NC_000117) Glycogen Synthase . . . | 170 | 6e−41 |
| gi\|3493107\|gb\|AAD03011.1\| | (AF079291) granule-bound starch s . . . | 167 | 4e−40 |
| gi\|3493007\|gb\|AAD02961.1\| | (AF079241) granule-bound starch s . . . | 166 | 6e−40 |
| gi\|3493103\|gb\|AAD03009.1\| | (AF079289) granule-bound starch s . . . | 166 | 6e−40 |
| gi\|7484399\|pir\|T07926 | probable starch synthase (EC 2.4.1.— . . . | 165 | 1e−39 |
| gi\|12278417\|gb\|AAG48949.1\| | (AY010962) granule-bound starch . . . | 164 | 2e−39 |
| gi\|3493005\|gb\|AAD02960.1\| | (AF079240) granule-bound starch s . . . | 164 | 2e−39 |
| gi\|3493111\|gb\|AAD03013.1\| | (AF079293) granule-bound starch s . . . | 164 | 3e−39 |
| gi\|15834801\|ref\|NP_296560.1\| | (NC_002620) glycogen synthase . . . | 164 | 3e−39 |
| gi\|12278503\|gb\|AAG48992.1\| | (AY011005) granule-bound starch . . . | 164 | 3e−39 |
| gi\|12278507\|gb\|AAG48994.1\| | (AY011007) granule-bound starch . . . | 164 | 4e−39 |
| gi\|3493117\|gb\|AAD03016.1\| | (AF079296) granule-bound starch s . . . | 163 | 5e−39 |
| gi\|3493079\|gb\|AAD02997.1\| | (AF079277) granule-bound starch s . . . | 163 | 5e−39 |
| gi\|12278509\|gb\|AAG48995.1\| | (AY011008) granule-bound starch . . . | 163 | 6e−39 |
| gi\|3493089\|gb\|AAD03002.1\| | (AF079282) granule-bound starch s . . . | 163 | 6e−39 |
| gi\|3493065\|gb\|AAD02990.1\| | (AF079270) granule-bound starch s . . . | 163 | 6e−39 |
| gi\|3493059\|gb\|AAD02987.1\| | (AF079267) granule-bound starch s . . . | 163 | 7e−39 |
| gi\|3493093\|gb\|AAD03004.1\| | (AF079284) granule-bound starch s . . . | 162 | 9e−39 |
| gi\|12278497\|gb\|AAG48989.1\| | (AY011002) granule-bound starch . . . | 162 | 9e−39 |
| gi\|3493113\|gb\|AAD03014.1\| | (AF079294) granule-bound starch s . . . | 162 | 9e−39 |
| gi\|12278505\|gb\|AAG48993.1\| | (AY011006) granule-bound starch . . . | 162 | 9e−39 |
| gi\|3493101\|gb\|AAD03008.1\| | (AF079288) granule-bound starch s . . . | 162 | 1e−38 |
| gi\|12278487\|gb\|AAG48984.1\| | (AY010997) granule-bound starch . . . | 162 | 1e−38 |
| gi\|3493061\|gb\|AAD02988.1\| | (AF079268) granule-bound starch s . . . | 162 | 1e−38 |
| gi\|3493049\|gb\|AAD02982.1\| | (AF079262) granule-bound starch s . . . | 162 | 1e−38 |
| gi\|3493019\|gb\|AAD02967.1\| | (AF079247) granule-bound starch s . . . | 162 | 1e−38 |
| gi\|12278514\|gb\|AAG48997.1\| | (AY011011) granule-bound starch . . . | 162 | 1e−38 |
| gi\|12278473\|gb\|AAG48977.1\| | (AY010990) granule-bound starch . . . | 162 | 2e−38 |
| gi\|3493121\|gb\|AAD03018.1\| | (AF079298) granule-bound starch s . . . | 162 | 2e−38 |
| gi\|12278481\|gb\|AAG48981.1\| | (AY010994) granule-bound starch . . . | 162 | 2e−38 |
| gi\|3493025\|gb\|AAD02970.1\| | (AF079250) granule-bound starch s . . . | 161 | 2e−38 |
| gi\|3493091\|gb\|AAD03003.1\| | (AF079283) granule-bound starch s . . . | 161 | 2e−38 |
| gi\|3493031\|gb\|AAD02973.1\| | (AF079253) granule-bound starch s . . . | 161 | 2e−38 |
| gi\|12278471\|gb\|AAG48976.1\| | (AY010989) granule-bound starch . . . | 161 | 2e−38 |
| gi\|12278463\|gb\|AAG48972.1\| | (AY010985) granule-bound starch . . . | 161 | 2e−38 |
| gi\|3493067\|gb\|AAD02991.1\| | (AF079271) granule-bound starch s . . . | 161 | 2e−38 |
| gi\|3493051\|gb\|AAD02983.1\| | (AF079263) granule-bound starch s . . . | 161 | 2e−38 |
| gi\|13774480\|gb\|AAK38879.1\| | (AF353517) granule-bound starch . . . | 161 | 3e−38 |
| gi\|12278453\|gb\|AAG48967.1\| | (AY010980) granule-bound starch . . . | 161 | 3e−38 |
| gi\|12278449\|gb\|AAG48965.1\| | (AY010978) granule-bound starch . . . | 161 | 3e−38 |
| gi\|12278451\|gb\|AAG48966.1\| | (AY010979) granule-bound starch . . . | 160 | 3e−38 |
| gi\|13774484\|gb\|AAK38881.1\| | (AF353519) granule-bound starch . . . | 160 | 3e−38 |
| gi\|12278427\|gb\|AAG48954.1\| | (AY010967) granule-bound starch . . . | 160 | 3e−38 |
| gi\|13774486\|gb\|AAK38882.1\| | (AF353520) granule-bound starch . . . | 160 | 3e−38 |
| gi\|12278491\|gb\|AAG48986.1\| | (AY010999) granule-bound starch . . . | 160 | 3e−38 |
| gi\|13774482\|gb\|AAK38880.1\| | (AF353518) granule-bound starch . . . | 160 | 4e−38 |
| gi\|13377473\|gb\|AAK20725.1\| | (AF318769) granule-bound starch . . . | 160 | 4e−38 |
| gi\|3493011\|gb\|AAD02963.1\| | (AF079243) granule-bound starch s . . . | 160 | 4e−38 |
| gi\|3493073\|gb\|AAD02994.1\| | (AF079274) granule-bound starch s . . . | 160 | 4e−38 |
| gi\|12278479\|gb\|AAG48980.1\| | (AY010993) granule-bound starch . . . | 160 | 5e−38 |
| gi\|3493057\|gb\|AAD02986.1\| | (AF079266) granule-bound starch s . . . | 160 | 6e−38 |
| gi\|3493087\|gb\|AAD03001.1\| | (AF079281) granule-bound starch s . . . | 160 | 6e−38 |
| gi\|3493023\|gb\|AAD02969.1\| | (AF079249) granule-bound starch s . . . | 160 | 6e−38 |
| gi\|3493041\|gb\|AAD02978.1\| | (AF079258) granule-bound starch s . . . | 160 | 6e−38 |
| gi\|3493109\|gb\|AAD03012.1\| | (AF079292) granule-bound starch s . . . | 160 | 6e−38 |
| gi\|3493081\|gb\|AAD02998.1\| | (AF079278) granule-bound starch s . . . | 160 | 6e−38 |
| gi\|3493021\|gb\|AAD02968.1\| | (AF079248) granule-bound starch s . . . | 160 | 6e−38 |
| gi\|12278457\|gb\|AAG48969.1\| | (AY010982) granule-bound starch . . . | 159 | 6e−38 |
| gi\|3493055\|gb\|AAD02985.1\| | (AF079265) granule-bound starch s . . . | 159 | 7e−38 |
| gi\|12278477\|gb\|AAG48979.1\| | (AY010992) granule-bound starch . . . | 159 | 7e−38 |
| gi\|12278499\|gb\|AAG48990.1\| | (AY011003) granule-bound starch . . . | 159 | 7e−38 |
| gi\|12278489\|gb\|AAG48985.1\| | (AY010998) granule-bound starch . . . | 159 | 7e−38 |
| gi\|12278511\|gb\|AAG48996.1\| | (AY011009) granule-bound starch . . . | 159 | 8e−38 |
| gi\|13774488\|gb\|AAK38883.1\| | (AF353521) granule-bound starch . . . | 159 | 8e−38 |
| gi\|12278485\|gb\|AAG48983.1\| | (AY010996) granule-bound starch . . . | 159 | 8e−38 |
| gi\|3493095\|gb\|AAD03005.1\| | (AF079285) granule-bound starch s . . . | 159 | 8e−38 |
| gi\|12278429\|gb\|AAG48955.1\| | (AY010968) granule-bound starch . . . | 159 | 8e−38 |
| gi\|3493077\|gb\|AAD02996.1\| | (AF079276) granule-bound starch s . . . | 159 | 8e−38 |
| gi\|3493009\|gb\|AAD02962.1\| | (AF079242) granule-bound starch s . . . | 159 | 8e−38 |
| gi\|3493027\|gb\|AAD02971.1\| | (AF079251) granule-bound starch s . . . | 159 | 1e−37 |
| gi\|12278413\|gb\|AAG48947.1\| | (AY010960) granule-bound starch . . . | 159 | 1e−37 |
| gi\|12278443\|gb\|AAG48962.1\| | (AY010975) granule-bound starch . . . | 159 | 1e−37 |
| gi\|3493097\|gb\|AAD03006.1\| | (AF079286) granule-bound starch s . . . | 159 | 1e−37 |
| gi\|3493013\|gb\|AAD02964.1\| | (AF079244) granule-bound starch s . . . | 159 | 1e−37 |
| gi\|3493015\|gb\|AAD02965.1\| | (AF079245) granule-bound starch s . . . | 159 | 1e−37 |
| gi\|3493099\|gb\|AAD03007.1\| | (AF079287) granule-bound starch s . . . | 159 | 1e−37 |
| gi\|3493083\|gb\|AAD02999.1\| | (AF079279) granule-bound starch s . . . | 159 | 1e−37 |
| gi\|13377475\|gb\|AAK20726.1\| | (AF318770) granule-bound starch . . . | 158 | 1e−37 |
| gi\|3493115\|gb\|AAD03015.1\| | (AF079295) granule-bound starch s . . . | 158 | 2e−37 |
| gi\|3493017\|gb\|AAD02966.1\| | (AF079246) granule-bound starch s . . . | 158 | 2e−37 |
| gi\|12278423\|gb\|AAG48952.1\| | (AY010965) granule-bound starch . . . | 158 | 2e−37 |
| gi\|12278425\|gb\|AAG48953.1\| | (AY010966) granule-bound starch . . . | 158 | 2e−37 |
| gi\|12278411\|gb\|AAG48946.1\| | (AY010959) granule-bound starch . . . | 158 | 2e−37 |
| gi\|12278435\|gb\|AAG48958.1\| | (AY010971) granule-bound starch . . . | 158 | 2e−37 |
| gi\|3493035\|gb\|AAD02975.1\| | (AF079255) granule-bound starch s . . . | 158 | 2e−37 |
| gi\|12278495\|gb\|AAG48988.1\| | (AY011001) granule-bound starch . . . | 158 | 2e−37 |
| gi\|12278501\|gb\|AAG48991.1\| | (AY011004) granule-bound starch . . . | 158 | 2e−37 |
| gi\|12278419\|gb\|AAG48950.1\| | (AY010963) granule-bound starch . . . | 158 | 2e−37 |
| gi\|12278421\|gb\|AAG48951.1\| | (AY010964) granule-bound starch . . . | 158 | 2e−37 |
| gi\|3493037\|gb\|AAD02976.1\| | (AF079256) granule-bound starch s . . . | 158 | 2e−37 |
| gi\|3493071\|gb\|AAD02993.1\| | (AF079273) granule-bound starch s . . . | 157 | 2e−37 |
| gi\|12278469\|gb\|AAG48975.1\| | (AY010988) granule-bound starch . . . | 157 | 3e−37 |
| gi\|3493003\|gb\|AAD02959.1\| | (AF079239) granule-bound starch s . . . | 157 | 3e−37 |
| gi\|3493001\|gb\|AAD02958.1\| | (AF079238) granule-bound starch s . . . | 157 | 3e−37 |
| gi\|13194734\|gb\|AAK15529.1\| | (AF331953) granule-bound starch . . . | 157 | 3e−37 |
| gi\|3493075\|gb\|AAD02995.1\| | (AF079275) granule-bound starch s . . . | 157 | 3e−37 |

Du-1

```
Accession: AF023159
            NID: g3057119
    Mol. wt. (calc) = 188101 Residues = 1674
```

Seq. ID. No. 883

| | |
|---|---|
| 1 | M E M V L R S Q S P L C L R S G P V L I F R P T V A G G G G |
| 31 | G T Q S L L R T T R F A R R R V I R C V V A S P G C P N R K |
| 61 | S R T A S P N V K V A A Y S N Y A P R L L V E S S S K K S E |
| 91 | H H D S S R H R E E T I D T Y N G L S G S D A A E L T S N R |
| 121 | D V E I E V D L Q H I S E E E L P G K V S I N A S L G E M E |
| 151 | T V D E A E V E E D K F E V D T S G I V L R N V A V R E V D |
| 181 | P K D E H N A K D V F V V D S S G T A P D N A A V E E V V D |
| 211 | E A E V E E D M V D V D I L G L D L N N A T I E E I D L M E |
| 241 | E A L L E N F D V D S P G N A S S G R T Y G G V D E L G E L |
| 271 | P S T S V D C I A I N G K R R S L K P K P L P I V R F Q E Q |
| 301 | E Q I V L S I V D E E G L I A S S C E E G Q P V V D Y D K Q |
| 331 | E E N S T A F D E Q K Q L T D D F P E E G I S I V H F P E P |
| 361 | N N D I V G S S K F L E Q K Q E L D G S Y K Q D R S T T G L |
| 391 | H E Q D Q S V V S S H G Q D K S I V G V P Q Q I Q Y N D Q S |
| 421 | I A G S H R Q D Q S I A G A P E Q I Q S V A G Y I K P N Q S |
| 451 | I V G S C K Q H E L I I P E P K K I E S I I S Y N E I D Q S |
| 481 | I V G S H K Q D K S V V S V P E Q I Q S I V S H S K P N Q S |
| 511 | T V D S Y R Q A E S I I G V P E K V Q S I T S Y D K L D Q S |
| 541 | I V G S L K Q D E P I I S V P E K I Q S I V H Y T K P N Q S |
| 571 | I V G L P K Q Q Q S I V H I V E P K Q S I D G F P K Q D L S |
| 601 | I V G I S N E F Q T K Q L A T V G T H D G L L M K G V E A K |
| 631 | E T S Q K T E G D T L Q A T F N V D N L S Q K Q E G L T K E |
| 661 | A D E I T I I E K I N D E D L V M I E E Q K S I A M N E E Q |
| 691 | T I V T E E D I P M A K V E I G I D K A K F L H L L S E E E |
| 721 | S S W D E N E V G I I E A D E Q Y E V D E T S M S T E Q D I |
| 751 | Q E S P N D D L D P Q A L W S M L Q E L A E K N Y S L G N K |
| 781 | L F T Y P D V L K A D S T I D L Y F N R D L S A V A N E P D |
| 811 | V L I K G A F N G W K W R F F T E K L H K S E L A G D W W C |
| 841 | C K L Y I P K Q A Y R M D F V F F N G H T V Y E N N N N N D |
| 871 | F V I Q I E S T M D E N L F E D F L A E E K Q R E L E N L A |
| 901 | N E E A E R R R Q T D E Q R R M E E E R A A D K A D R V Q A |
| 931 | K V E V E T K K N K L C N V L G L A R A P V D N L W Y I E P |
| 961 | I T T G Q E A T V R L Y Y N I N S R P L V H S T E I W M H G |
| 991 | G Y N N W I D G L S F A E R L V H H H D K D C D W W F A D V |
| 1021 | V V P E R T Y V L D W V F A D G P P G S A R N Y D N N G G H |
| 1051 | D F H A T L P N N M T E E E Y W M E E E Q R I Y T R L Q Q E |
| 1081 | R R E R E E A I K R K A E R N A K M K A E M K E K T M R M F |
| 1111 | L V S Q K H I V Y T E P L E I H A G T T I D V L Y N P S N T |
| 1141 | V L T G K P E V W F R C S F N R W M Y P G G V L P P Q K M V |
| 1171 | Q A E N G S H L K A T V Y V P R D A Y M M D F V F S E S E E |
| 1201 | G G I Y D N R N G L D Y H I P V F G S I A K E P P M H I V H |
| 1231 | I A V E M A P I A K V G G L G D V V T S L S R A V Q D L G H |
| 1261 | N V E V I L P K Y G C L N L S N V K N L Q I H Q S F S W G G |
| 1291 | S E I N V W R G L V E G L C V Y F L E P Q N G M F G V G Y V |
| 1321 | Y G R D D D R R F G F F C R S A L E F L L Q S G S S P N I I |
| 1351 | H C H D W S S A P V A W L H K E N Y A K S S L A N A R V V F |
| 1381 | T I H N L E F G A H H I G K A M R Y C D K A T T V S N T Y S |
| 1411 | K E V S G H G A I V P H L G K F Y G I L N G I D P D I W D P |
| 1441 | Y N D N F I P V H Y T C E N V V E G K R A A K R A L Q Q K F |
| 1471 | G L Q Q I D V P V V G I V T R L T A Q K G I H L I K H A I H |
| 1501 | R T L E R N G Q V V L L G S A P D S R I Q A D F V N L A N T |
| 1531 | L H G V N H G Q V R L S L T Y D E P L S H L I Y A G S D F I |
| 1561 | L V P S I F E P C G L T Q L V A M R Y G T I P I V H K T G G |
| 1591 | L F D T V F D V D N D K E R A R D R G L E P N G F S F D G A |
| 1621 | D S N G V D Y A L N R A I S A W F D A R S W F H S L C K R V |
| 1651 | M E Q D W S W N R P A L D Y I E L Y R S A S K L |

TABLE XXXI
Maize soluble starch synthase IIb (SSIIb)
Alignments with other similar proteins-Transit Peptide

| SEQ | Accession | a.a | | a.a. |
|---|---|---|---|---|
| Id.No. | Number | # | (start) Sequence ending | # |
| 884 | MAIZE SSIIb | 1 | GGIYDNRNGLDYHIPVFGSIAKEP----- P-------MHIVHIAVEMAPIAKVGGLGD | 46 |
| 885 | 7489826 | 1201 | GGIYDNRNGLDYHIPVFGSIAKEP---- -P-------MHIVHIAVEMAPIAKVGGLGD | 1246 |
| 886 | 9502145 | 1139 | GIYDNRNGMDYHIPVSDSIETEN-------Y-------MRIIHIAVEMAPVAKVGGLGD | 1183 |
| 887 | 9502143 | 1156 | GIYDNRNGMDYHIPVSDSIETEN-------Y-------MRIIHIAVEMAPVAKVGGLGD | 1200 |
| 888 | 17646328 | 743 | GGIYDNRNGMDYHSPVTDSVAKEP------P-------MHIVHIAVEMAPIAKVGGLGD | 788 |
| 889 | 4582789 | 672 | GGVFDNKFGMDYHIPVFGGIVKEP------P-------MHIVHIAVEMAPIAKVGGLGD | 717 |
| 890 | 7489274 | 755 | GGIFDNKSGMDYHIPVFGGVAKEP------P-------MHIVHIAVEMAPIAKVGGLGD | 800 |
| 891 | 2833389 | 755 | GGIFDNKSGMDYHIPVFGGVAKEP------P-------MHIVHIAVEMAPIAKVGGLGD | 800 |
| 892 | 15221083 | 552 | GGIFDNKNGLDYHLPVVGGISKEP-------P-------LHIVHIAVEMAPIAKVGGLGD | 597 |
| 893 | 4582783 | 370 | SSATSP-------G-------LYVIHIAAEMAPVAKVGGLGD | 397 |
| 894 | 15717885 | 421 | -------LHIAHIAAEMAPVAKVGGLAD | 441 |
| 895 | 15236819 | 545 | -------LYVVHIAAEMAPVAKVGGLGD | 565 |
| 896 | 18461221 | 348 | VLQVGGLAD | 356 |
| 897 | 8901183 | 129 | MPLQGGAAEAPPVERDGNP-------MHIIHITAEMAPIAKVGGLGD | 168 |
| 898 | 17227527 | 1 | -------MYIVQIASECAPVIKAGGLGD | 21 |
| 899 | 16329217 | 1 | -------MYIVQIASECAPVIKAGGLGD | 21 |
| 900 | 2811062 | 1 | -------MKVLFAVSECAPFAKSGGLAD | 21 |
| 901 | 18309046 | 15 | EANPFIKTGGLGD | 27 |
| 902 | 15903076 | 1 | -------MKILFVAAEGAPFSKTGGLGD | 21 |
| 903 | 17229371 | 1 | -------MRILFVAAEAAPIAKVGGMGD | 21 |
| 904 | 15900991 | 1 | -------MKILFVAAEGAPFSKTGGLGD | 21 |
| 905 | 16124067 | 1 | -------MRVLHVCSELFPLLKTGGLAD | 21 |
| 906 | 16080147 | 1 | -------MKILFAVSECTPFVKSGGLAD | 21 |
| 907 | 16273270 | 1 | -------MKILHVCSELYPLLKTGGLAD | 21 |
| 908 | 15602409 | 1 | -------MKVLHACSELYPLLKTGGLAD | 21 |
| 909 | 15672681 | 2 | KER-------K-------MKVLFASSECAPFFKTGGLGD | 26 |
| 910 | 17938870 | 22 | -------MRILAVTAEMFPFVKTGGLAD | 42 |
| 911 | 16119514 | 1 | -------MRILAVTAEMFPFVKTGGLAD | 21 |
| 912 | 15613648 | 1 | -------MNVLHVASECNPFFKTGGLAD | 21 |
| 913 | 16331219 | 1 | -------MKILFVAAEVSPLAKVGGMGD | 21 |
| 914 | 17366711 | 1 | -------MKVLFASSECAPFFKTGGLGD | 21 |
| 915 | 15805621 | 28 | -------P-------MRVLHLASEVFPFSRSGGLGD | 49 |
| 916 | 15620537 | 1 | -------MRILFVAAECAPFAKVGGMGD | 21 |
| 917 | 15641730 | 12 | EVQGLVKSGGLAD | 24 |
| 918 | 7489711 | 199 | GPLAGPN-------V-------MNVVVVASECAPFCKTGGLGD | 227 |
| 919 | 15605532 | 1 | -------MKIIHTAIEFAPVIKAGGLGD | 21 |
| 920 | 15803938 | 1 | -------MQVLHVCSEMFPLLKTGGLAD | 21 |
| 921 | 17367076 | 1 | -------MRVLHLASEVFPFSRSGGLGD | 21 |
| 922 | 16766821 | 1 | -------MQVLHVCSEMFPLLKTGGLAD | 21 |
| 923 | 16762766 | 1 | -------MQVLHVCSEMFPLLKTGGLAD | 21 |
| 924 | 15834801 | 1 | -------MKITHTAIEFAPVIKAGGLGD | 21 |
| 925 | 15966599 | 1 | -------MNILSVASEVYPLVKTGGLAD | 21 |
| 926 | 15618857 | 1 | -------MRIVQVAVEFTPIVKVGGLGD | 21 |
| 927 | 17366350 | 2 | -------IRVLHLAPEAYPLAKVGGLAD | 22 |
| 928 | 15384987 | 138 | GPLAGPN-------V-------MNVIVVASECSPFCKTGGLGD | 166 |
| 929 | 2129898 | 245 | FESGGEKP-------PPLAGTNVMNIILVSAECAPWSKTGGLGD | 281 |
| 930 | 2833384 | 245 | FESGGEKP-------PPLAGTNVMNIILVSAECAPWSKTGGLGD | 281 |
| 931 | 15028467 | 195 | GPLAGPN-------V-------MNVIVVASECSPFCKTGGLGD | 223 |
| 932 | 15643657 | 1 | -------MKVVFVSYEVFPFAKVGGLAD | 21 |

TABLE XXXII
Maize soluble starch synthase III (Du I)
“GLASS” Domain Alignments with other similar proteins

| SEQ | Accession | a.a | | a.a. |
|---|---|---|---|---|
| Id.No. | Number | # | (start) Sequence end | # |
| 933 | MaizeDuI | 47 | VVTSLSRAVQDLGH--NVEVIL--PKY----GCL------N--LSNV-------KNL--- | 80 |
| 934 | 7489826 | 1247 | VVTSLSRAVQDLGH--NVEVIL--PKY----GCL------N--LSNV-------KNL--- | 1280 |
| 935 | 9502145 | 1184 | VVTSLSRAVQDLGH--TVEVIL--PKY----DCL------N--QSSV-------KDL--- | 1217 |
| 936 | 9502143 | 1201 | VVTSLSRAIQDLGH--TVEVIL--PKY----DCL------N--QSSV-------KDL--- | 1234 |
| 937 | 17646328 | 789 | VVTSLSRAVQDLGH--NVEVIL--PKY----DCL------N--LSNV-------KDL--- | 822 |
| 938 | 4582789 | 718 | VVTSLSRAVQDLNH--NVDIIL--PKY----DCL------N--LSNV-------KDL--- | 751 |
| 939 | 7489274 | 801 | VVTSLSRAVQDLNH--NVDIIL--PKY----DCL------K--MNNV-------KDF--- | 834 |
| 940 | 2833389 | 801 | VVTSLSRAVQDLNH--NVDIIL--PKY----DCL------K--MNNV-------KDF--- | 834 |
| 941 | 15221083 | 598 | VVTSLSRAVQELNH--NVDIVF--PKY----DCI------K--HNFV-------KDL--- | 631 |
| 942 | 4582783 | 398 | VISGLSKALQKKGH--LVEIIL--PKY----DCM------Q--YDRI-------GDL--- | 431 |
| 943 | 15717885 | 442 | VISGLGKALQKKGH--LVEIIL--PKY----DCM------Q--VDQV-------SNL--- | 475 |
| 944 | 15236819 | 566 | VVAGLGKALQRKGH--LVEIIL--PKY----DCM------Q--YDRV-------RDL--- | 599 |
| 945 | 18461221 | 357 | VVAGLGKALQTKGH--LVEIVL--PKY----DCM------Q--LDQI-------TNL--- | 390 |
| 946 | 8901183 | 169 | VVTGLAKAALARGH--FVTVML--PFY----ECL------P--KDQI-------EGLKHE | 205 |
| 947 | 17227527 | 22 | VVYGLSRELEIRGN--CVELIL--PKY----DCM------R--YDHV-------WGL--- | 55 |
| 948 | 16329217 | 22 | VIYGLSRELELRGH--CVELIL--PMY----DCM------R--YDHIWGLHDAYRNL--- | 62 |
| 949 | 2811062 | 22 | VAGALPKELRRLGI--DARVML--PKY----ETIAPEWKKK--MKKV-------AEL--- | 61 |
| 950 | 18309046 | 28 | VMGALPKELKRKGI--DARVIL--PKY----SAI------K--GELL-------DKL--- | 61 |
| 951 | 15903076 | 22 | VIGALPKSLVKAGH--EVAVIL--PYY----DMVEAEFGNQ--IEDV-------LEF--- | 61 |
| 952 | 17229371 | 22 | VVGALPKVLRKMGH--DVRIFL--PYY----GFL------P-------------DKM--- | 51 |
| 953 | 15900991 | 22 | VIGALPKSLVKAGH--EVAVIL--PYY----DMVEAKFGNQ--IEDV-------LHF--- | 61 |
| 954 | 16124067 | 22 | VIGALPAAQLAEGA--DVRIIL--PAF----PDL------R--RGIP-------ETV--- | 55 |
| 955 | 16080147 | 22 | VAGALPKALARLGN--EVAVML--PKY----SQI------P--EPWK-------KEN--- | 55 |
| 956 | 16273270 | 22 | VLGALPQAQNQIGL--DARFLL--PAY----PAI------T---TGI-------PNT--- | 54 |
| 957 | 15602409 | 22 | VMGALPFAQKEIGI--DARIVL--PAY----PAI------K---AGI-------PET--- | 54 |
| 958 | 15672681 | 27 | VAGALPKELAKKSEIDSVAVIL--PYF----KNE------M--KEEY-------RSL--- | 62 |
| 959 | 17938870 | 43 | ATSALPQALERKGA--DVRTLL--PLY----RGL------TPLVRGR-------KPV--- | 78 |
| 960 | 16119514 | 22 | ATSALPQALERKGA--DVRTLL--PLY----RGL------TPLVRGR-------KPV--- | 57 |
| 961 | 15613648 | 22 | VLGSLPKALIKQGI--NVSVIL--PKY----GHL------S--DEWQ-------NQL--- | 55 |
| 962 | 16331219 | 22 | VVGSLPKVLHQLGH--DVRVFM--PYY----GFI------G-------------DKI--- | 51 |
| 963 | 17366711 | 22 | VAGALPKELAKKSEIDSVAVIL--PYF----KNE------M--KEEY-------RSL--- | 57 |
| 964 | 15805621 | 50 | VLGALPAVQARLGE--DAEVTVLSPWY----ASL-----------QG-------EPQ--- | 82 |
| 965 | 15620537 | 22 | VVGSLPKVLKASAH--DVGIFM--RYY----GFL------------T-------SKL--- | 51 |
| 966 | 15641730 | 25 | VAKALPQALKALHQ--QVAIAL--PAY----RSV------P--GKED-------AEL--- | 58 |
| 967 | 7489711 | 228 | VVGALPKALARRGH--RVMVVI--PRY----G--------E--YAEA-------RDL--- | 259 |
| 968 | 15605532 | 22 | ALYGLAKALA-ANH--TTEVVI--PLY----PKLF-----T--LPKE-------QDL--- | 55 |
| 969 | 15803938 | 22 | VIGALPAAQIADGV--DARVLL--PAF----PDI------R---RGV-------TDA--- | 54 |
| 970 | 17367076 | 22 | VLGALPAVQARLGE--DAEVTVLSPWY----ASL-----------QG-------EPQ--- | 54 |
| 971 | 16766821 | 22 | VIGALPAAQIADGV--DVRVLL--PGF----PDI------R--RGIP-------DAH--- | 55 |
| 972 | 16762766 | 22 | VIGALPAAQIADGV--DVRVLL--PGF----PDI------R--RGIP-------DAH--- | 55 |
| 973 | 15834801 | 22 | ALYGLAKALA-VNH--TTEVVI--PLYPKLFTSL------H--EQDL-------FAT--- | 58 |
| 974 | 15966599 | 22 | VVGALPSALLPHGV--RTRTLV--PGY----PSV------L--KKLK-------KKK--- | 55 |
| 975 | 15618857 | 22 | AVASLSKELAK-QN--DVEVLL--PHY----PLI------S--KFSS-------SQV--- | 54 |
| 976 | 17366350 | 23 | VVGALPKALRPLGV--EAHVLL--PWH----GGL------E--ARRV-------GEV--- | 56 |
| 977 | 15384987 | 167 | VVGALPKALARRGH--RVMVVI--PRY----G--------E--YAEA-------KDL--- | 198 |
| 978 | 2129898 | 282 | VAGSLPKALARRGH--RVMIVA--PHY----G--------N--YAEA-------HDI--- | 313 |
| 979 | 2833384 | 282 | VAGSLPKALARRGH--RVMIVA--PHY----G--------N--YAEA-------HDI--- | 313 |
| 980 | 15028467 | 224 | VVGALPKALARRGH--RVMVVI--PRY----G--------E--YAEA-------KDL--- | 255 |
| 981 | 15643657 | 22 | VAGTLPKYLKKHGV--DVTIVM--PKH----RIV------E---KNA-------EKFGYE | 57 |
| 933 | maizeDu1 | 81 | ---Q-----I-H----Q----SF-----S-----W---G----G----S------EI--N | 94 |
| 934 | 7489826 | 1281 | ---Q-----I-H----Q----SF-----S-----W---G----G----S------EI--N | 1294 |
| 935 | 9502145 | 1218 | ---H-----L-Y----Q----SF-----S-----W---G----G----T------EI--K | 1231 |
| 936 | 9502143 | 1235 | ---H-----L-Y----Q----SF-----S-----W---G----G----T------EI--K | 1248 |
| 937 | 17646328 | 823 | ---H-----Y-R----Q----SF-----T-----W---G----N----T------EI--K | 836 |
| 938 | 4582789 | 752 | ---Q-----F-H----K----SY-----F-----W---S----G----T------EI--K | 765 |
| 939 | 7489274 | 835 | ---R-----F-H----K----NY-----F-----W---G----G----T------EI--K | 848 |
| 940 | 2833389 | 835 | ---H-----F-H----K----NY-----F-----W---G----G----T------EI--K | 848 |
| 941 | 15221083 | 632 | ---Q-----F-N----R----SY-----H-----W---G----G----T------EI--K | 645 |
| 942 | 4582783 | 432 | ---RALDVVI-E----S----YF-----D-----G---Q----L----F------KN--K | 450 |
| 943 | 15717885 | 476 | ---K-----V-LDVLVQ----SY-----F-----E---G----N----M------FN--N | 493 |
| 944 | 15236819 | 600 | ---RALDTVV-E----S----YF-----D-----G---K----L----Y------KM--K | 618 |
| 945 | 18461221 | 391 | ---KVLDVVI-Q----S----YF-----D-----G---N----L----F------SN--N | 409 |
| 946 | 8901183 | 206 | CDIE-----V-P----K----GY-----H-----W---D----G----EIRVGPLKT--S | 228 |
| 947 | 17227527 | 56 | -----------H----E----AYLNLWVP-----W---F----G----A------AI--H | 72 |
| 948 | 16329217 | 63 | ---E-----V------------------P-----W---Y----G----S------SIFCD | 74 |
| 949 | 2811062 | 62 | ---I-----V-----------PV-----G-----W---R----R----Q------YC--G | 73 |
| 950 | 18309046 | 62 | ---S-----F-K----K----WFMVPV-G-----W---R----N----Q------YC--G | 79 |
| 952 | 15903076 | 62 | ---E-----V-----------SV-----G-----W---R----R----Q------YC--G | 73 |
| 952 | 17229371 | 52 | ---E-----I-P----K----DP-----I-----W---K----GYAMFQ------DF--T | 69 |
| 953 | 15900991 | 62 | ---E-----V-----------SV-----G-----W---R----R----Q------YC--G | 73 |
| 954 | 16124067 | 56 | ---L-----V-R----E----ID-----T-----F---A----G-----------RV--A | 68 |
| 955 | 16080147 | 56 | ---K-----K-Q----AECTVAV-----G-----W---R----Q----Q------YC--G | 73 |
| 956 | 16273270 | 55 | ---Q-----V-V----A----EF-----D-----N---F----A----G------HV--V | 68 |
| 957 | 15602409 | 55 | ---T-----V-V----S----EF-----D-----N---F----A----G------HI--V | 68 |
| 958 | 15672681 | 63 | ---L-----K-D----E----FY-----DFVDVGW---R----H----E------YV--G | 81 |
| 959 | 17938870 | 79 | ---L-----V-A----N----IL-----E-------------------Q------PV--S | 89 |
| 960 | 16119514 | 58 | ---L-----V-A----N----IL-----E-------------------Q------PV--S | 68 |
| 961 | 15613648 | 56 | ---T-----L-K----K----SF-----T-----V---S----V----T------WR--N | 69 |
| 962 | 16331219 | 52 | ---D-----V-P----K----EP-----V-----W---K----G---------------- | 61 |
| 963 | 17366711 | 58 | ---L-----K-D----E----FY-----DFVDVGW---R----H----E------YV--G | 76 |
| 964 | 15805621 | 83 | ---E-----L-W----R----GE-----A-----W---G----E---------------Q | 93 |
| 965 | 15620537 | 52 | ---D-----IPA----E----PI-----W-----W---G----Y----A------MF--N | 66 |
| 966 | 15641730 | 59 | ---V-----L-E----T----EL-----TH----W---P----H----T------QY--R | 73 |
| 967 | 74897112 | 60 | ---G-----V-R----R----RY-----K-----V---A----GQD--S------EV--T | 275 |
| 968 | 15605532 | 56 | ---C-----S-I----Q----KL-----S-----YFFAG----E----Q------EA--T | 72 |
| 969 | 15803938 | 55 | ---Q-----V-V----S----RR-----D-----T---F----A----G------HI--T | 68 |
| 970 | 17367076 | 55 | ---E-----L-W----R----GE-----A-----W---G----E---------------Q | 65 |
| 971 | 16766821 | 56 | ---V-----V-S----R----RD-----T-----F---A----G-----------KI--S | 68 |
| 972 | 16762766 | 56 | ---V-----V-S----R----RD-----T-----F---A----G-----------KI--S | 68 |
| 973 | 15834801 | 59 | ---Q-----K-I----P----YF-----F-----A---G----E----Q------EA--T | 72 |
| 974 | 15966599 | 56 | ---p-----V-G----R----FD-----N-----L---F----G----H------PA--T | 69 |
| 975 | 15618857 | 55 | ---L-----S-E----R----SF-----Y-----YEFLG----K----Q------QA--S | 71 |
| 976 | 17366350 | 57 | ---------------------AF-----A-----F---F----G----R------EE--R | 66 |
| 977 | 15384987 | 199 | ---G-----V-R----K----RY-----R-----V---A----GQD--S------EV--S | 214 |
| 978 | 2129898 | 314 | ---G-----V-R----K----RY-----K-----V---A----G----QDM----EV--T | 329 |
| 979 | 2833384 | 314 | ---G-----V-R----K----RY-----K-----V---A----G----QDM----EV--T | 329 |
| 980 | 15028467 | 256 | ---G-----V-R----K----RY-----R-----V---A----GQD--S------EV--S | 271 |
| 981 | 15643657 | 58 | IK-K-----V-A----E----SL-----S-----V---SHVKTD----Q------KF--D | 77 |
| 933 | Maize Du1 | 95 | ---VWRG-LV----EG--LCV--Y--F--LE-----P-----------Q---NG---M-- | 114 |
| 934 | 7489826 | 1295 | ---VWRG-LV----EG--LCV--Y--F--LE-----P-----------Q---NG---M-- | 1314 |
| 935 | 9502145 | 1232 | ---VWVG-RV----ED--LTV--Y--F--LE-----P-----------Q---NG---M-- | 1251 |
| 936 | 9502143 | 1249 | ---VWVG-RV----ED--LTV--Y--F--LE-----P-----------Q---NG---M-- | 1268 |
| 937 | 17646328 | 837 | ---VWFG-KV----ED--VPV--Y--F--LE-----P-----------Q---NG---M-- | 856 |
| 938 | 4582789 | 766 | ---VWHG-KV----EG--LSV--Y--F--LE-----P-----------Q---NG---LF- | 786 |
| 939 | 7489274 | 849 | ---VWFG-KV----EG--LSV--Y--F--LE-----P-----------Q---NG---LF- | 869 |
| 940 | 2833389 | 849 | ---VWFG-KV----EG--LSV--Y--F--LE-----P-----------Q---NG---LF- | 869 |
| 941 | 15221083 | 646 | ---VWHG-KV----EG--LSV--Y--F--LD-----P-----------Q---NG---LF- | 666 |
| 942 | 4582783 | 451 | ---IWVG-TV----EG--LPV--Y--F--IE-----P-----------H---HPGKFF-- | 473 |
| 943 | 15717885 | 494 | K--IWTG-TV----EG--LPV--Y--F--IE-----P-----------Q---HPAMFF-- | 517 |
| 944 | 15236819 | 619 | ---IWIG-TV----EG--LPV--H--F--IE-----P-----------Q---HPSKFF-- | 641 |
| 945 | 18461221 | 410 | ---VWTG-TV----EG--LPV--Y--F--IE-----P-----------Q---HPSKFF-- | 432 |
| 946 | 8901183 | 229 | ---VFWG-RV----QG--CPV--Y--L--IK-----P-----------A---DD---TNC | 250 |
| 947 | 17227527 | 73 | CT-VYCG-WV----HG--RVC--F--F--IE-----P-----------HSEDNF---F-- | 97 |
| 948 | 16329217 | 75 | ---VFCG-WV----HG--RLC--F--F--IQ-----P-----------KSSDNF---F-- | 97 |
| 949 | 2811062 | 74 | ---VEEL-RH----DG--VIY--Y--F--ID-----N-----------E---YY---F-- | 93 |
| 950 | 18309046 | 80 | ---VYQC-EY----DE--VIY--Y--L--LD-----S-----------E---FY---F-- | 99 |
| 951 | 15903076 | 74 | ---IKKT-VL----NG--VTF--Y--F--ID-----N-----------Q---YY---F-- | 93 |
| 952 | 17229371 | 70 | ---VHEA-VL----PG--TDVP-L--Y--LF-----G-----------H---PA---F-- | 90 |
| 953 | 15900991 | 74 | ---IKKT-VL----NG--VTF--Y--F--ID-----N-----------Q---YY---F-- | 93 |
| 954 | 16124067 | 69 | ---LRYG-HY----RG--IGI--Y--L--ID-----APALYDRAGSPYH---DA---S-- | 99 |
| 955 | 16080147 | 74 | ---IEHM-AE----ND--VNY--Y--F--ID-----N-----------E---YY---F-- | 93 |
| 956 | 16273270 | 69 | ---LRYG-EY----NG--VGI--Y--L--ID-----A-----------P---HL---Y-- | 88 |
| 957 | 15602409 | 69 | ---LRYG-EY----KG--LGV--Y--L--IDA----P-----------H---LY---Q-- | 89 |
| 958 | 15672681 | 82 | ---VKSL-EK----EG--VKY--Y--F--LD-----N-----------E---HY---F-- | 101 |
| 959 | 17938870 | 90 | ---VYYV-VS----DG--HKL--L--L--LE-----A-----------A---SL---F-- | 109 |
| 960 | 16119514 | 69 | ---VYYV-VS----DG--HKL--L--L--LE-----A-----------A---SL---F-- | 88 |
| 961 | 15613648 | 70 | ---QYCG-LEQFVDQG--VTY--Y--F--ID-----N-----------E---YY---F-- | 93 |
| 962 | 16331219 | 62 | -----EA-MF----QQ--FAV--YQSY--L------P-----------D---TK---I-- | 80 |
| 963 | 17366711 | 77 | ---VKSL-EK----EG--VKY--Y--F--LD-----N-----------E---HY---F-- | 96 |
| 964 | 15805621 | 94 | ---AWLG-EL----RQ--DGV--R--Y--LF-----L-----------G---LN---E-- | 113 |
| 965 | 15620537 | 67 | HFAVYET-QL----PG--SDVP-L--Y--LM-----G-----------H---PA---9-- | 90 |
| 966 | 15641730 | 74 | ---VLKR-DL----DG--VPI--Y--L--IDCPAYFD-----------R---PA---L-- | 98 |
| 967 | 7489711 | 276 | ---YFHS-YI----DG--VDF--V--F--VE-----APPF--------R---HR---H-- | 298 |
| 968 | 15605532 | 73 | ---AFSY-FY----EG--IKVTLF--K--LD-----T-----------Q---PE---L-- | 94 |
| 969 | 15803938 | 69 | ---LLFG-HY----NG--VGI--Y--L--ID-----A-----------P---H------- | 86 |
| 970 | 17367076 | 66 | ---AWLG-EL----RQ--DGV--R--Y--LF-----L-----------G---LN---E-- | 85 |
| 971 | 16766821 | 69 | ---LLFG-HY----NG--VGI--Y--L--IDA----P-----------H---LY---E-- | 89 |
| 972 | 16762766 | 69 | ---LLWG-HY----NG--VGI--Y--L--IDA----P-----------H---LY---S-- | 89 |
| 973 | 15834801 | 73 | ---AFSY-FY----EG--IKV--T--LLKLD-----S-----------Q---PE---L-- | 94 |
| 974 | 15966599 | 70 | ---VLAA-EV----NG--VDL--L--V--LD-----Q-----------P---AL---Y-- | 89 |
| 975 | 15618857 | 72 | ---AISY-SY----EGLTLTI--I--T--LD-----S-----------Q---IE---L-- | 93 |
| 976 | 17366350 | 67 | ---APLGERV----EG--GVR--F--L--LL-----G-----------V---EG---F-- | 87 |
| 977 | 15384987 | 215 | ---YFHA-FI----DG--VDF--V--F--LE-----APPF--------R---HR---H-- | 237 |
| 978 | 2129898 | 330 | ---YFHT-YI----DG--VDI--V--F--ID-----SPIF--------R---NL---E-- | 352 |
| 979 | 2833384 | 330 | ---YFHT-YI----DG--VDI--V--F--ID-----SPIF--------R---NL---E-- | 352 |
| 980 | 15028467 | 272 | ---YFHA-FI----DG--VDF--V--F--LE-----APPF--------R---HR---H-- | 294 |
| 981 | 15643657 | 78 | ---IYES-VL----PG--SDVKTY--F--VA-----N-----------D---YY---F-- | 99 |
| 933 | maizeDu-1 | 115 | ----XXXXXXXXXXXXXXXXXXCR---------SA--------L--------E---FL-L | 141 |
| 934 | 7489826 | 1315 | ----FGVGYVYGRDDDRRFGFFCR---------SA--------L--------E---FL-L | 1341 |
| 935 | 9502145 | 1252 | ----FGVGCVYGRNDDRRFGFFCH---------SA--------L--------E---FI-L | 1278 |
| 936 | 9502143 | 1269 | ----FGVGCVYGRNDDRRFGFFCH---------SA--------L--------E---FI-L | 1295 |
| 937 | 17646328 | 857 | ----FWVGCVYGRNDESRFGFFCH---------SA--------L--------E---FL-R | 883 |
| 938 | 4582789 | 787 | ----NVGCVYGRANDAERFGFFCH---------AA--------L--------E---FL-L | 813 |
| 939 | 7489274 | 870 | ----SKGCVYGCSNDGERFGFFCR---------AA--------L--------E---FL-L | 896 |
| 940 | 2833389 | 870 | ----SKGCVYGCSNDGERFGFFCH---------AA--------L--------E---FL-L | 896 |
| 941 | 15221083 | 667 | ----QRGCVYGCADDAGRFGFFCH---------AA--------L--------E---FL-L | 693 |
| 942 | 4582783 | 474 | ----WRGDYYGAHDDFRRFSYFSR---------AA--------L--------E---FL-L | 500 |
| 943 | 15717885 | 518 | ----SRAQYYGEHDDFKRFSYFSR---------AA--------L--------E---LL-Y | 544 |
| 944 | 15236819 | 642 | ----WRGQFYGEQDDFRRFSYFSR---------AA--------L--------E---LL-L | 668 |
| 945 | 18461221 | 433 | ----WRAQYYGEHDDFKRYSYFSR---------AA--------L--------E---LL-Y | 459 |
| 946 | 8901183 | 251 | NIFRGGRIYGGSYNEMEAYLYFCR---------AC--------L--------E---YL-N | 281 |
| 947 | 17227527 | 98 | ----NRGCYYGCDDDDMRFAFFSK---------AA--------L--------E---FL-H | 124 |
| 948 | 16329217 | 98 | ----NRGHYYGALDDHMRFAFFSK---------AA--------M--------E---FL-L | 124 |
| 949 | 2811062 | 94 | ----KRPQLYGHYDDGERFAYFCR---------AV--------L--------E---VL-P | 120 |
| 982 | 7484399 | 62 | CR---------AC--------L--------E---YLNV | 71 |
| 950 | 18309046 | 100 | ----HRNGLYGEGDDGERFAFFDR---------AV--------L--------E---TL-K | 126 |
| 951 | 15903076 | 94 | ----FRGHVYGDFDDGERFAFFQL---------AA--------I--------E---AM-E | 120 |
| 952 | 17229371 | 91 | ----TPRRIYSGDDEDWRFTLFSN---------GA--------A--------E---FC-W | 117 |
| 953 | 15900991 | 94 | ----FRGHVYGDFDDGERFAFFQL---------AA--------I--------E---AM-E | 120 |
| 954 | 16124067 | 100 | ----LYAYSDNYLRFALLGWMACE---------LA--------C--------G------L | 124 |
| 955 | 16080147 | 94 | ----NRDSLYGHYDDGERFAFFSR---------AV--------L--------E---AA-K | 120 |
| 956 | 16273270 | 89 | ----GREGNPYHDAYYNDYGDNYKRFALLGWVGAE--------L--------A---TG-L | 124 |
| 957 | 15602409 | 90 | ----REGNPYHDQWYNDYADNYKR---------FALLGWVAAEL--------A---TG-L | 124 |
| 958 | 15672681 | 102 | ----GRGQLYGYGDDGERFAFFDL---------AL--------C--------Q---LL-E | 128 |
| 959 | 17938870 | 110 | ----DRDGHPYGVKGEPFADNDLR---------FA--------VLSKVAA--E---IA-L | 142 |
| 960 | 16119514 | 89 | ----DRDGHPYGVKGEPFADNDLR---------FA--------VLSKVAA--E---IA-L | 121 |
| 961 | 15613648 | 94 | ----KRERLYGYLDEAERFTFFNH---------AV--------L--------S---SL-P | 120 |
| 962 | 16331219 | 81 | ----PLYLFGHPAFDSRRIYGGDD---------EA--------W--------R---FT-F | 107 |
| 963 | 17366711 | 97 | ----GRGQLYGYGDDGERFAFFDL---------AL--------C--------Q---LL-E | 123 |
| 964 | 15805621 | 114 | ----FQRPGLYHPDDVERFCAFGR---------AA--------L--------P---AL-D | 140 |
| 965 | 15620537 | 91 | ----DPHRIYSGEDEDWRFTFFAN---------GA--------A--------E---FS-W | 117 |
| 966 | 15641730 | 99 | ----YAENNQAYADNGERFGFFSA---------AC--------L--------D---VL-P | 125 |
| 967 | 7489711 | 299 | ----NNIYGGERLDILKRMILFCK---------AA--------V--------E---VP-W | 325 |
| 968 | 15605532 | 95 | ----FENAETIYTSDDAFRFCAFS---------AA--------A--------A---SY-I | 121 |
| 969 | 15803938 | 87 | -----LYDRPGSPYHDTNLFAYTD---------NV--------L--------R---FA-L | 112 |
| 970 | 17367076 | 86 | ----FQRPGLYHPDDVERFCAFGR---------AA--------L--------P---AL-D | 112 |
| 971 | 16766821 | 90 | ----RPGSPYHDTNLYAYTDNVLR---------FA--------LLGWVGCEMA---CG-L | 124 |
| 972 | 16762766 | 90 | ----RPGSPYHDTNLYAYIDNVLR---------FA--------LLGWVGCEMA---CG-L | 124 |
| 973 | 15834801 | 95 | ----FEEAETIYTNDDAFRFCAFS---------AA--------A--------A---AY-I | 121 |
| 974 | 15966599 | 90 | ----ARDGGPYLDSTGRDYPDNFR---------RF--------A--------A---LS-L | 116 |
| 975 | 15618857 | 94 | ----FSTTSVYSENNVVRFSAFAA---------AA--------A--------A---Y--L | 119 |
| 976 | 17366350 | 88 | ----GRERVYGYPDDAERYLRFAL---------AA--------K--------E---V--- | 112 |
| 977 | 15384987 | 238 | ----NDIYGGERFDVLKRMILFCK---------AA--------V--------EVPWFA-P | 267 |
| 978 | 2129898 | 353 | ----SNIYGGNRLDILRRMVLFCK---------AA--------V--------E---VP-W | 379 |
| 979 | 2833384 | 353 | ----SNIYGGNRLDILRRMVLFCK---------AA--------V--------E---VP-W | 379 |
| 980 | 15028467 | 295 | ----NDIYGGERFDVLKRMILFCK---------AA--------V--------EVPWFA-P | 324 |
| 981 | 15643657 | 100 | ----SAEDVYAGPDLGEQAIFFCA---------AT--------L--------D---LV-K | 126 |
| 933 | MaizeDu1 | 142 | --------QS-------GS----------S---------PNIIH-CNDWSS--A--PV-A | 161 |
| 934 | 7489826 | 1342 | --------QS-------GS----------S---------PNIIH-CHDWSS--A--PV-A | 1361 |
| 935 | 9502145 | 1279 | --------QN-------EF----------S---------PHIIH-CHDWSS--A--PV-A | 1298 |
| 936 | 9502143 | 1296 | --------QN-------EF----------S---------PHIIH-CHDWSS--A--PV-A | 1315 |
| 937 | 17646328 | 884 | --------QN-------GS----------S---------PDIIH-CHDWSS--A--PV-A | 903 |
| 938 | 4582789 | 814 | --------QN-------GS----------H---------PDIIH-CHDWSS--A--PV-A | 833 |
| 939 | 7489274 | 897 | --------QG-------GF----------S---------PDIIH-CHDWSS--A--PV-A | 916 |
| 940 | 2833389 | 897 | --------QG-------GF----------S---------PDIIH-CHDWSS--A--PV-A | 916 |
| 942 | 15221083 | 694 | --------QG-------GF----------H---------PDILH-CHDWSS--A--PV-S | 713 |
| 942 | 4582783 | 501 | --------QA-------GK----------K---------PDIIH-CHDWQT--A--FI-A | 520 |
| 943 | 15717885 | 545 | --------QS-------GK----------K---------VDIIH-CHDWQT--A--FV-A | 564 |
| 944 | 15236819 | 669 | --------QS-------GK----------K---------PDIIH-CHDWQT--A--FV-A | 688 |
| 945 | 18461221 | 460 | --------QS-------GK----------K---------IDIIH-CHDWQT--A--FV-A | 479 |
| 946 | 8901183 | 282 | --------VS-------QQ----------N---------PHVLQ-LHDWHA--A--AA-S | 301 |
| 947 | 17227527 | 125 | --------QS-------NK----------R---------PDIIH-CHDWQT--G--LI-P | 144 |
| 948 | 16329217 | 125 | --------RS-------NK----------R---------PDIIH-CHDWQT--G--LV-P | 144 |
| 949 | 2811062 | 121 | --------EI-------QF----------Q---------PDVIH-CHDWHT--G--MV-P | 140 |
| 982 | 7484399 | 72 | --------SQ-------QQ----------N---------PHVLQ-LHDWHA--A--AA-S | 91 |
| 950 | 18309046 | 127 | --------EI-------DW----------C---------PDIIH-CNDWQT--G--MI-P | 146 |
| 951 | 15903076 | 121 | --------RI-------DF----------I---------PDLLH-VHDYHT--A--MI-P | 140 |
| 952 | 17229371 | 118 | --------NY-------W-----------K---------PDIIH-CHDWHT--G--MIPV | 137 |
| 953 | 15900991 | 121 | --------RI-------DF----------I---------PDLLH-VHDYHT--A--MI-P | 140 |
| 954 | 16124067 | 125 | --------DG-------YW----------R---------PEVVH-AHDWHA--G--LT-C | 144 |
| 955 | 16080147 | 121 | --------VV-------NV----------Q---------ADIVH-THDWHT--A--MV-N | 140 |
| 956 | 16273270 | 125 | --------DS-------WW----------R---------AEVVH-AHDWHA--GL-CV-A | 145 |
| 957 | 15602409 | 125 | --------DS-------WW----------H---------ADVVH-AHDWHAGLA--SA-Y | 146 |
| 958 | 15672681 | 129 | --------KL-------DF----------I---------PDVLH-VNDWQT--A--MV-P | 148 |
| 959 | 17938870 | 143 | --------GAID-----GW----------Q---------PDVVH-VHDWHA--A--LT-C | 164 |
| 960 | 16119514 | 122 | --------GAID-----GW----------Q---------PDVVH-VHDWHA--A--LT-C | 143 |
| 961 | 15613648 | 121 | --------FL-------DE----------S---------PDLIH-CHDWQS--G--LI-P | 140 |
| 962 | 16331219 | 108 | --------FS-------NG----------AAEFAWNHWKPEIIH-CHDWHT--G--MIPV | 137 |
| 963 | 17366711 | 124 | --------KL-------DF----------I---------PDVLH-VNDWQT--A--MV-P | 143 |
| 964 | 15805621 | 141 | --------AV-------GV----------T---------PDVLH-GHDWQA--G--LV-V | 160 |
| 965 | 15620537 | 118 | --------NY-------W-----------K---------PQVIH-CHDWHT--G--MIPV | 137 |
| 966 | 15641730 | 126 | --------KL-------GI----------Q---------PDIIH-ANDWHT--G--LV-P | 145 |
| 967 | 7489711 | 326 | --------YAPCGGTVYGD----------G---------NLVFI-ANDWHT--A--LL-P | 352 |
| 968 | 15605532 | 122 | --------QK--------E----------G---------ANIVH-LHDWHT--G--LV-A | 140 |
| 969 | 15803938 | 113 | LGWVGAEMAS-------GL----------DPFWR-----PDVVH-AHDWHAGLA--PA-Y | 146 |
| 970 | 17367076 | 113 | --------AV-------GV----------T---------PDVLH-GHDWQA--G--LV-V | 132 |
| 971 | 16766821 | 125 | --------DP-------FW----------R---------PDVVH-AHDWHAGLA--PA-Y | 146 |
| 972 | 16762766 | 125 | --------DP-------FW----------R---------PDVVH-AHDWHAGLA--PA-Y | 146 |
| 973 | 15834801 | 122 | --------QK--------E----------G---------ADIVH-LHDWHV--G--LV-A | 140 |
| 974 | 15966599 | 117 | --------AA-------AEIAGNGIIPNWK---------PDIVH-VHDWQT--ALTPV-- | 147 |
| 975 | 15618857 | 120 | --------QE-------AD----------P---------ADIVH-LHDWHV--G--LL-A | 139 |
| 976 | 17366350 | 113 | -----------------AR----------G---------YDLVH-AHDWTA--A--LL-A | 130 |
| 977 | 15384987 | 268 | --------CG-------GSIYG-------D---------GNLVFIANDWHT--A--LL-P | 291 |
| 978 | 2129898 | 380 | --------HVPCGGICYGD----------G---------NLVFI-ANDWHT--A--LL-P | 406 |
| 979 | 2833384 | 380 | --------HVPCGGICYGD----------G---------NLVFI-ANDWHT--A--LL-P | 406 |
| 980 | 15028467 | 325 | --------CG-------GSIYG-------D---------GNLVFIANDWHT--A--LL-P | 348 |
| 981 | 15643657 | 127 | --------HL-------DL----------K---------PDIVH-VNDWQT--A--LI-P | 146 |
| 933 | Maize Du1 | 162 | WLHKENYA-KSS-L-A-N-ARVV---------------FTIHN----------------- | 184 |
| 934 | 7489826 | 1362 | WLHKENYA-KSS-L-A-N-ARVV---------------FTIHN----------------- | 1384 |
| 935 | 9502145 | 1299 | WLYKEHYS-QSR-M-A-S-TRVV---------------FTIHN----------------- | 1321 |
| 936 | 9502143 | 1316 | WLYKEHYS-QSR-M-A-S-TRVV---------------FTIHN----------------- | 1338 |
| 937 | 17646328 | 904 | WLFKEQYA-QNG-L-S-N-GRVV---------------FTIHN----------------- | 926 |
| 938 | 4582789 | 834 | WLFKEQYT-HYG-L-S-K-ARVV---------------FTIHN----------------- | 856 |
| 939 | 7489274 | 917 | WLFKEQYT-HYG-L-S-K-SRIV---------------FTIHN----------------- | 939 |
| 940 | 2833389 | 917 | WLFKEQYT-HYG-L-S-K-SRIV---------------FTIHN----------------- | 939 |
| 941 | 15221083 | 714 | WLFKDHYT-QYG-L-I-K-TRIV---------------FTIHN----------------- | 736 |
| 942 | 4582783 | 521 | PLYWDVYA-FKG-L-N-S-ARIC---------------FTCHN----------------- | 543 |
| 943 | 15717885 | 565 | PLYWDVYA-NLG-F-N-S-ARIC---------------FTCHN----------------- | 587 |
| 944 | 15236819 | 689 | PLYWDLYA-PKG-L-D-S-ARIC---------------FTCHN----------------- | 711 |
| 945 | 18461221 | 480 | PLYWDIYA-TRG-F-S-S-ARIC---------------FTCHN----------------- | 502 |
| 946 | 8901183 | 302 | MLYWDVYN-PNG-F-S-R-TRLM---------------LTIHN----------------- | 324 |
| 947 | 17227527 | 145 | VMLYEIYK-YHG-M-D-T-QRVC---------------YTIHN----------------- | 167 |
| 948 | 16329217 | 145 | VLLYEIYR-FHG-M-D-H-QRVC---------------YTIHN----------------- | 167 |
| 949 | 2811062 | 141 | FLLREQYR-HEL-FYV-D-MRTV---------------FTIHN----------------- | 164 |
| 982 | 7484399 | 92 | MLYWDVYN-PNG-F-S-R-TRLM---------------LTIHN----------------- | 114 |
| 950 | 18309046 | 147 | VLHKLEYS-KDP-FYK-N-IKTV---------------TSIHN----------------- | 170 |
| 951 | 15903076 | 141 | FLLKEKYRWIQA-Y-E-D-IETV---------------LTIHN----------------- | 164 |
| 952 | 17229371 | 138 | WMNQS----------P-D-ITTV---------------FTIHN----------------- | 153 |
| 953 | 15900991 | 141 | FLLKEKYRWIQA-Y-E-D-IETV---------------LTIHN----------------- | 164 |
| 954 | 16124067 | 145 | -----AYL-AAR-G-R-P-ARSV---------------FTVHN----------------- | 162 |
| 955 | 16080147 | 141 | YLLKEEYR-KHP-FYE-R-MKSV---------------LTIHN----------------- | 164 |
| 956 | 16273270 | 146 | YLFNKGKP-----------AKSV---------------FTIHN----------------- | 162 |
| 957 | 15602409 | 147 | LFNKGRPA-KS-----------V---------------FTIHN----------------- | 162 |
| 958 | 15672681 | 149 | FLLKEKYNWIKA-Y-E-K-IKTV---------------LTIHN----------------- | 172 |
| 959 | 17938870 | 165 | V-----YL-ADS-T-P-S-VASV---------------LTLHN----------------- | 182 |
| 960 | 16119514 | 144 | V-----YL-ADS-T-P-S-VASV---------------LTLHN----------------- | 161 |
| 961 | 15613648 | 141 | AYMKTGSV-ENP-V-P-----TV---------------FTIHNLRYQGAFPPDVFREL-- | 175 |
| 962 | 16331219 | 138 | WMHQS----------P-D-IATV---------------FTIHN----------------- | 153 |
| 963 | 17366711 | 144 | FLLKEKYNWIKA-Y-E-K-IKTV---------------LTIHN----------------- | 167 |
| 964 | 15805621 | 161 | -------A-HAR-L-R-G-LRTA---------------FSVHN----------------- | 176 |
| 965 | 15620537 | 138 | WMHQS----------P-D-ISTV---------------FTIHN----------------- | 153 |
| 966 | 15641730 | 146 | FLLKTRYR-YDSFF-E-Q-VKSV---------------LTVHNAIFKGIFSYHQLEVIPE | 186 |
| 967 | 7489711 | 353 | VYLKAYYR-DNG-L-M-QYARSV---------------LVIHN----------------- | 376 |
| 968 | 15605532 | 141 | GLLKQQPC--SQ-L-----QKIV---------------LTLHN----------------- | 160 |
| 969 | 15803938 | 147 | LAARGRPA-KS-----------V---------------FTVHN----------------- | 162 |
| 970 | 17367076 | 133 | -------A-HAR-L-R-G-LRTA---------------FSVHN----------------- | 148 |
| 971 | 16766821 | 147 | LAARGRPA-KS-----------V---------------FTVHN----------------- | 162 |
| 972 | 16762766 | 147 | LAARGRPA-KS-----------V---------------FTVHN----------------- | 162 |
| 973 | 15834801 | 141 | GLLKQQPC-PQL-------QKIV---------------LTLHN----------------- | 160 |
| 974 | 15966599 | 148 | ------YM-RFG-P-APD-LPTV---------------MTIHN----------------- | 165 |
| 975 | 15618857 | 140 | GLLKNPL----N-P-V-H-SKIV---------------FTIHN----------------- | 159 |
| 976 | 17366350 | 131 | LYAPTVYT-IHN-L-A-H-QGLVDPGLFFSWTGLPWSLFHMEA----------------- | 168 |
| 977 | 15384987 | 292 | VYLKAYYR-DNG-L-M-QYTRSV---------------LVIHNIAHQGRGPVDDFAT--- | 329 |
| 978 | 2129898 | 407 | VYLKAYYR-DHG-L-M-NYTRSV---------------LVIHN----------------- | 430 |
| 979 | 2833384 | 407 | VYLKAYYR-DHG-L-M-NYTRSV---------------LVIHN----------------- | 430 |
| 980 | 15028467 | 349 | VCLKAYYR-DNG-L-M-QYTRSV---------------LVIHNIAHQGRGPVDDFAT--- | 386 |
| 981 | 15643657 | 147 | VYLKTVYR-DDPYF-S-R-TATV---------------LTIHN----------------- | 170 |
| 933 | maizeDu1 | 185 | --------L-----------------E--------------------------------- | 186 |
| 934 | 7489826 | 1385 | --------L-----------------E--------------------------------- | 1386 |
| 935 | 9502145 | 1322 | --------L-----------------E--------------------------------- | 1323 |
| 936 | 9502143 | 1339 | --------L-----------------E--------------------------------- | 1340 |
| 937 | 17646328 | 927 | --------L-----------------E--------------------------------- | 928 |
| 938 | 4582789 | 857 | --------L-----------------E--------------------------------- | 858 |
| 939 | 7489274 | 940 | --------L-----------------E--------------------------------- | 941 |
| 940 | 2833389 | 940 | --------L-----------------E--------------------------------- | 941 |
| 941 | 15221083 | 737 | --------L-----------------E--------------------------------- | 738 |
| 942 | 4582783 | 544 | --------L-----------------E--------------------------------- | 545 |
| 943 | 15717885 | 588 | --------L-----------------E--------------------------------- | 589 |
| 944 | 15236819 | 712 | --------L-----------------E--------------------------------- | 713 |
| 945 | 18461221 | 503 | --------L-----------------E--------------------------------- | 504 |
| 946 | 8901183 | 325 | --------L-----------------D--------------------------------- | 326 |
| 947 | 17227527 | 168 | --------F-----------------KHQGIGGVKTLWATGLNREAYYFQDDKLRDDHNP | 202 |
| 948 | 16329217 | 168 | --------F-----------------E--------------------------------- | 169 |
| 949 | 2811062 | 165 | --------L-----------------E--------------------------------- | 166 |
| 982 | 7484399 | 115 | --------L-----------------E--------------------------------- | 116 |
| 950 | 18309046 | 171 | --------L-----------------E--------------------------------- | 172 |
| 951 | 15903076 | 165 | --------L-----------------E--------------------------------- | 166 |
| 952 | 17229371 | 154 | --------L-----------------E--------------------------------- | 155 |
| 953 | 15900991 | 165 | --------L-----------------E--------------------------------- | 166 |
| 954 | 16124067 | 163 | --------L-----------------AYQGL----------------------------- | 168 |
| 955 | 16080147 | 165 | --------L-----------------E--------------------------------- | 166 |
| 956 | 16273270 | 163 | --------LAYQG-------------Q--------------------------------- | 168 |
| 957 | 15602409 | 163 | --------LAYQG-------------Q--------------------------------- | 168 |
| 958 | 15672681 | 173 | --------I-----------------E--------------------------------- | 174 |
| 959 | 17938870 | 183 | --------L-----------------A--------------------------------- | 184 |
| 960 | 16119514 | 162 | --------L-----------------A--------------------------------- | 163 |
| 961 | 15613648 | 176 | --------L-----------------H--------------------------------- | 177 |
| 962 | 16331219 | 154 | --------L-----------------A--------------------------------- | 155 |
| 963 | 17366711 | 168 | --------I-----------------E--------------------------------- | 169 |
| 964 | 15805621 | 177 | --------L-----------------QYQGRWNLHEAAGWTGLPDWTFGPDGVEF----- | 206 |
| 965 | 15620537 | 154 | --------L-----------------A--------------------------------- | 155 |
| 966 | 15641730 | 187 | LNLSGMEFL-----------------Q--------------------------------- | 196 |
| 967 | 7489711 | 377 | --------IAHQGRGPVDDFVNF---D--------------------------------- | 392 |
| 968 | 15605532 | 161 | --------F-----------------G--------------------------------- | 162 |
| 969 | 15803938 | 163 | --------L-----------------AYQGM----------------------------- | 168 |
| 970 | 17367076 | 149 | --------L-----------------QYQGRWNLHEAAGWTGLPDWTFGPDGVEF----- | 178 |
| 971 | 16766821 | 163 | --------L-----------------A--------------------------------- | 164 |
| 972 | 16762766 | 163 | --------L-----------------A--------------------------------- | 164 |
| 973 | 15834801 | 161 | --------FGYRGYTTREVLEASSLNE--------------------------------- | 179 |
| 974 | 15966599 | 166 | --------IAFQG-------------Q--------------------------------- | 171 |
| 975 | 15618857 | 160 | --------FGYRGYCSTQLLAASQIDD--------------------------------- | 178 |
| 976 | 17366350 | 169 | --------L-----------------E--------------------------------- | 170 |
| 977 | 15384987 | 330 | --------M-----------------D--------------------------------- | 331 |
| 978 | 2129898 | 431 | --------I-----------------AHQGRGPVEDFNTVDLSGNYLDLFKMYDP----- | 460 |
| 979 | 2833384 | 431 | --------I-----------------AHQGRGPVEDFNTVDLSCNYLDLFKMYDP----- | 460 |
| 980 | 15028467 | 387 | --------M-----------------D--------------------------------- | 388 |
| 981 | 15643657 | 171 | --------L-----------------G--------------------------------- | 172 |

TABLE XXXIII
Maize soluble starch synthase III (Du I)
“LINKR” Domain Alignments with other similar proteins

| SEQ | Accession | a.a | | a.a. |
|---|---|---|---|---|
| Id.No. | Number | # | (start) Sequence end | # |
| 983 | MaizeDuI | 187 | F-----------------------------GA---------------------------- | 189 |
| 984 | 7489826 | 1387 | F-----------------------------GA---------------------------- | 1389 |
| 985 | 9502145 | 1324 | F-----------------------------GA---------------------------- | 1326 |
| 986 | 9502143 | 1341 | F-----------------------------GA---------------------------- | 1343 |
| 987 | 17646328 | 929 | F-----------------------------GA---------------------------- | 931 |
| 988 | 4582789 | 859 | F-----------------------------GA---------------------------- | 861 |
| 989 | 7489274 | 942 | F-----------------------------GA---------------------------- | 944 |
| 990 | 2833389 | 942 | F-----------------------------GA---------------------------- | 944 |
| 991 | 15221083 | 739 | F-----------------------------GA---------------------------- | 741 |
| 992 | 4582783 | 546 | YQGTAGASELEACGLDSHQLNRPDRMQDN-SA---------------------------- | 576 |
| 993 | 15717885 | 590 | YQGTAPARDLAWCGL---------------DV---------------------------- | 606 |
| 994 | 15236819 | 714 | YQGTA-------------------------SA---------------------------- | 720 |
| 995 | 18461221 | 505 | Y-----------------------------QG---------------------------- | 507 |
| 996 | 8901183 | 327 | N-----------------------------TG---------------------------- | 329 |
| 997 | 17227527 | 203 | F-----------------------------AL---------------------------- | 205 |
| 998 | 16329217 | 170 | HQGIA-------------------------GANIL------------------------- | 179 |
| 999 | 2811062 | 167 | F-----------------------------QGLFPRGILEDLLNLDGRYFTVDHLEFYGC | 197 |
| 1000 | 7484399 | 117 | N-----------------------------TG---------------------------- | 119 |
| 1001 | 18309046 | 173 | FQGNFSADVLPELFGYDYEPVRNGSLEFYGGM---------------------------- | 204 |
| 1002 | 15903076 | 167 | F-----------------------------QG---------------------------- | 169 |
| 1003 | 17229371 | 156 | YQGPWRWYLDKITWCPWYMQ----------GH---------------------------- | 177 |
| 1004 | 15900991 | 167 | F-----------------------------QG---------------------------- | 169 |
| 1005 | 16124067 | 169 | F-----------------------------SA---------------------------- | 171 |
| 1006 | 16080147 | 167 | F-----------------------------QGIFPPDVT--------------------- | 176 |
| 1007 | 16273270 | 169 | F-----------------------------SY---------------------------- | 171 |
| 1008 | 15602409 | 169 | F-----------------------------AY---------------------------- | 171 |
| 1009 | 15672681 | 175 | F-----------------------------QG---------------------------- | 177 |
| 1010 | 17938870 | 185 | FQ----------------------------GQ---------------------------- | 188 |
| 1011 | 16119514 | 164 | FQ----------------------------GQ---------------------------- | 167 |
| 1012 | 15613648 | 178 | F-----------------------------AP---------------------------- | 180 |
| 1013 | 16331219 | 156 | YQGPWRGLLETMTWCPWYMQ----------GD---------------------------- | 177 |
| 1014 | 17366711 | 170 | F-----------------------------QG---------------------------- | 172 |
| 1015 | 15805621 | 207 | F-----------------------------GD---------------------------- | 209 |
| 1016 | 15620537 | 156 | YQGPWRWKLEKITWCPWYMQ----------GD---------------------------- | 177 |
| 1017 | 15641730 | 197 | Y-----------------------------GH---------------------------- | 199 |
| 1018 | 7489711 | 393 | L-----------------------------PE---------------------------- | 395 |
| 1019 | 15605532 | 163 | Y-----------------------------RG---------------------------- | 165 |
| 1020 | 15803938 | 169 | F-----------------------------YA---------------------------- | 171 |
| 1021 | 17367076 | 179 | F-----------------------------GD---------------------------- | 181 |
| 1022 | 16766821 | 165 | Y-----------------------------QG---------------------------- | 167 |
| 1023 | 16762766 | 165 | Y-----------------------------QG---------------------------- | 167 |
| 1024 | 15834801 | 180 | F-----------------------------YL---------------------------- | 182 |
| 1025 | 15966599 | 172 | F-----------------------------GA---------------------------- | 174 |
| 1026 | 15618857 | 179 | F-----------------------------HL---------------------------- | 181 |
| 1027 | 17366350 | 171 | FY----------------------------GR---------------------------- | 174 |
| 1028 | 15384987 | 332 | L-----------------------------PE---------------------------- | 334 |
| 1029 | 2129898 | 461 | V-----------------------------GG---------------------------- | 463 |
| 1030 | 2833384 | 461 | V-----------------------------GG---------------------------- | 463 |
| 1031 | 15028467 | 389 | L-----------------------------PE---------------------------- | 391 |
| 1032 | 15643657 | 173 | YQGVFDPKYLSFAGLPDYVYTID-------GL---------------------------- | 197 |
| 983 | maizeDu1 | 190 | -H------------------------H-----------------------------I--- | 192 |
| 984 | 7489826 | 1390 | -H------------------------H-----------------------------I--- | 1392 |
| 985 | 9502145 | 1327 | -H------------------------Y-----------------------------I--- | 1329 |
| 986 | 9502143 | 1344 | -H------------------------Y-----------------------------I--- | 1346 |
| 987 | 17646328 | 932 | -H------------------------H-----------------------------I--- | 934 |
| 988 | 4582789 | 862 | -N------------------------L-----------------------------I--- | 864 |
| 989 | 7489274 | 945 | -D------------------------L-----------------------------I--- | 947 |
| 990 | 2833389 | 945 | -D------------------------L-----------------------------I--- | 947 |
| 991 | 15221083 | 742 | -N------------------------A-----------------------------I--- | 744 |
| 992 | 4582783 | 577 | -H------------------------NRVNS-------------------------V--- | 583 |
| 993 | 15717885 | 607 | -E------------------------H-----------------------------L--- | 609 |
| 994 | 15236819 | 721 | -S------------------------E-----------------------------L--- | 723 |
| 995 | 18461221 | 508 | -T------------------------A-----------------------------P--- | 510 |
| 996 | 8901183 | 330 | -ETRQDEFFFTGVPGENFA-------T-----------------------------I--- | 349 |
| 997 | 17227527 | 206 | -N------------------------Y-----------------------------M--- | 208 |
| 998 | 16329217 | 180 | -H------------------------A-----------------------------T--- | 182 |
| 999 | 2811062 | 198 | VS------------------------F-----------------------------M--- | 201 |
| 1000 | 7484399 | 120 | -ETRQDEFFFTGVPGENFA-------T-----------------------------I--- | 139 |
| 1001 | 18309046 | 205 | -S------------------------F-----------------------------M--- | 207 |
| 1002 | 15903076 | 170 | -Q------------------------FSEGMLGDLFGVGFERYADGTLRWNNCLNWM--- | 201 |
| 1003 | 17229371 | 178 | -N------------------------T-----------------------------M--- | 180 |
| 1004 | 15900991 | 170 | -Q------------------------FSEGMLGDLFGVGFERYADGTLRWNNCLNWM--- | 201 |
| 1005 | 16124067 | 172 | -D------------------------H-----------------------------L--- | 174 |
| 1006 | 16080147 | 177 | -H------------------------DL----------------------------L--- | 180 |
| 1007 | 16273270 | 172 | -H------------------------HLYEIGLPTGMFHVEGLELFGQISY-----L--- | 198 |
| 1008 | 15602409 | 172 | -H------------------------HLVEIGLPAGMFHVDGLELHGQISY-----L--- | 198 |
| 1009 | 15672681 | 178 | -L------------------------M-----------------------------H--- | 180 |
| 1010 | 17938870 | 189 | -Y------------------------P-----------------------------L--- | 191 |
| 1011 | 16119514 | 168 | -Y------------------------P-----------------------------L--- | 170 |
| 1012 | 15613648 | 181 | -E------------------------HFAGLEMDGAINF-----------------L--- | 195 |
| 1013 | 16331219 | 178 | -N------------------------V-----------------------------M--- | 180 |
| 1014 | 17366711 | 173 | -L------------------------M-----------------------------H--- | 175 |
| 1015 | 15805621 | 210 | -L------------------------N-----------------------------L--- | 212 |
| 1016 | 15620537 | 178 | -S------------------------T-----------------------------M--- | 180 |
| 1017 | 15641730 | 200 | -D------------------------H-----------------------------V--- | 202 |
| 1018 | 7489711 | 396 | -H------------------------Y-----------------------------IDHF | 401 |
| 1019 | 15605532 | 166 | -Y------------------------TTREILEASSLNEFYISQYQLFRDPQTCV-L--- | 196 |
| 1020 | 15803938 | 172 | -H------------------------H-----------------------------M--- | 174 |
| 1021 | 17367076 | 182 | -L------------------------N-----------------------------L--- | 184 |
| 1022 | 16766821 | 168 | -M------------------------F-----------------------------Y--- | 170 |
| 1023 | 16762766 | 168 | -M------------------------F-----------------------------Y--- | 170 |
| 1024 | 15834801 | 183 | -S------------------------HYQLFRDPQTCV------------------L--- | 196 |
| 1025 | 15966599 | 175 | -SVFPELALPPDAFSTQFVEYYGDVGF-----------------------------L--- | 201 |
| 1026 | 15618857 | 182 | -S------------------------HYQLFRDPQTSV------------------L--- | 195 |
| 1027 | 17366350 | 175 | -V------------------------N-----------------------------L--- | 177 |
| 1028 | 15384987 | 335 | -H------------------------Y-----------------------------IDHF | 340 |
| 1029 | 2129898 | 464 | -E------------------------HFNI--------------------------F--- | 469 |
| 1030 | 2833384 | 464 | -E------------------------HFNI--------------------------F--- | 469 |
| 1031 | 15028467 | 392 | -H------------------------Y-----------------------------IDF- | 397 |
| 1032 | 15643657 | 198 | -E------------------------F-----------------------------Y--- | 200 |
| 983 | maize DuI | 193 | --------------G-------------------K------------------------- | 194 |
| 984 | 7489826 | 1393 | --------------G-------------------K------------------------- | 1394 |
| 985 | 9502145 | 1330 | --------------G-------------------K------------------------- | 1331 |
| 986 | 9502143 | 1347 | --------------G-------------------K------------------------- | 1348 |
| 987 | 17646328 | 935 | --------------G-------------------K------------------------- | 936 |
| 988 | 4582789 | 865 | --------------G-------------------K------------------------- | 866 |
| 989 | 7489274 | 948 | --------------G-------------------R------------------------- | 949 |
| 990 | 2833389 | 948 | --------------G-------------------R------------------------- | 949 |
| 991 | 15221083 | 745 | --------------G-------------------K------------------------- | 746 |
| 992 | 4582783 | 584 | --------------K-------------------G------------------------- | 585 |
| 993 | 15717885 | 610 | --------------D-------------------RPDRMRDNSHGRINAVKG-------- | 628 |
| 994 | 15236819 | 724 | --------------G-------------------SCGLDVNQLNRPDRMQDHSSGDRVNP | 750 |
| 995 | 18461221 | 511 | --------------A-------------------P------------------------- | 512 |
| 996 | 8901183 | 350 | --------------D-------------------K------------------------- | 351 |
| 997 | 17227527 | 209 | --------------K-------------------G------------------------- | 210 |
| 998 | 16329217 | 183 | --------------GLNNDSYYFSYDRLQDNFNPN------------------------- | 203 |
| 999 | 2811062 | 202 | --------------K-------------------G------------------------- | 203 |
| 1000 | 7484399 | 140 | --------------D-------------------K------------------------- | 141 |
| 1001 | 18309046 | 208 | --------------K-------------------G------------------------- | 209 |
| 1002 | 15903076 | 202 | --------------K-------------------A------------------------- | 203 |
| 1003 | 17229371 | 181 | --------------A-------------------A------------------------- | 182 |
| 1004 | 15900991 | 202 | --------------K-------------------A------------------------- | 203 |
| 1005 | 16124067 | 175 | --------------S-------------------ELHLPAEFFQIYGLEFYGQISYLKA- | 200 |
| 1006 | 16080147 | 181 | --------------G-------------------L------------------------- | 182 |
| 1007 | 16273270 | 199 | --------------K-------------------S------------------------- | 200 |
| 1008 | 15602409 | 199 | --------------K-------------------A------------------------- | 200 |
| 1009 | 15672681 | 181 | --------------G-------------------D------------------------- | 182 |
| 1010 | 17938870 | 192 | --------------E-------------------R------------------------- | 193 |
| 1011 | 16119514 | 171 | --------------E-------------------R------------------------- | 172 |
| 1012 | 15613648 | 196 | --------------K-------------------G------------------------- | 197 |
| 1013 | 16331219 | 181 | --------------A-------------------A------------------------- | 182 |
| 1014 | 17366711 | 176 | --------------G-------------------D------------------------- | 177 |
| 1015 | 15805621 | 213 | --------------M-------------------K------------------------- | 214 |
| 1016 | 15620537 | 181 | --------------A-------------------A------------------------- | 182 |
| 1017 | 15641730 | 203 | --------------SMLR----------------A------------------------- | 207 |
| 1018 | 7489711 | 402 | KLYDNIGGDHSNVFA-------------------A------------------------- | 417 |
| 1019 | 15605532 | 197 | --------------L-------------------K------------------------- | 198 |
| 1020 | 15803938 | 175 | --------------N-------------------DIQLPWSFFNIHGLEFNGQISFLKA- | 200 |
| 1021 | 17367076 | 185 | --------------M-------------------K------------------------- | 186 |
| 1022 | 16766821 | 171 | --------------A-------------------K------------------------- | 172 |
| 1023 | 16762766 | 171 | --------------A-------------------K------------------------- | 172 |
| 1024 | 15834801 | 197 | --------------L-------------------K------------------------- | 198 |
| 1025 | 15966599 | 202 | --------------K-------------------G------------------------- | 203 |
| 1026 | 15618857 | 196 | --------------M-------------------K------------------------- | 197 |
| 1027 | 17366350 | 178 | --------------M-------------------KG------------------------ | 180 |
| 1028 | 15384987 | 341 | RLYDPVGGEHSNVFA-------------------A------------------------- | 356 |
| 1029 | 2129898 | 470 | --------------A-------------------A------------------------- | 471 |
| 1030 | 2833384 | 470 | --------------A-------------------A------------------------- | 471 |
| 1031 | 15028467 | 398 | RLYDPVGGEHSNVFA-------------------A------------------------- | 413 |
| 1032 | 15643657 | 201 | --------------G-------------------QLNFLKG------------------- | 208 |
| 983 | maize-Du1 | 195 | ---A--------------MR-----------------------------Y---------- | 198 |
| 984 | 7489826 | 1395 | ---A--------------MR-----------------------------Y---------- | 1398 |
| 985 | 9502145 | 1332 | ---A--------------MT-----------------------------Y---------- | 1335 |
| 986 | 9502143 | 1349 | ---A--------------MT-----------------------------Y---------- | 1352 |
| 987 | 17646328 | 937 | ---A--------------MA-----------------------------R---------- | 940 |
| 988 | 4582789 | 867 | ---A--------------MA-----------------------------Y---------- | 870 |
| 989 | 7489274 | 950 | ---A--------------MT-----------------------------N---------- | 953 |
| 990 | 2833389 | 950 | ---A--------------MT-----------------------------N---------- | 953 |
| 991 | 15221083 | 747 | ---A--------------MT-----------------------------F---------- | 750 |
| 992 | 4582783 | 586 | ---A--------------VV-----------------------------Y---------- | 589 |
| 993 | 15717885 | 629 | ---A--------------VV-----------------------------Y---------- | 632 |
| 994 | 15236819 | 751 | VKGA--------------II-----------------------------F---------- | 757 |
| 995 | 18461221 | 513 | ---D--------------LS-----------------------------Y---------- | 516 |
| 996 | 8901183 | 352 | ---A--------------LDERTIGHNPERLNLMKGGIV----------Y---------- | 374 |
| 997 | 17227527 | 211 | ---G--------------IV-----------------------------Y---------- | 214 |
| 998 | 16329217 | 204 | ---A--------------IN-----------------------------F---------- | 207 |
| 999 | 2811062 | 204 | ---A--------------LV-----------------------------A---------- | 207 |
| 1000 | 7484399 | 142 | ---A--------------LDERTIGHNPERLNLMKGGIV----------Y---------- | 164 |
| 1001 | 18309046 | 210 | ---A--------------IN-----------------------------Y---------- | 213 |
| 1002 | 15903076 | 204 | ---G--------------IL-----------------------------Y---------- | 207 |
| 1003 | 17229371 | 183 | ---A--------------VQ-----------------------------F---------- | 186 |
| 1004 | 15900991 | 204 | ---G--------------IL-----------------------------Y---------- | 207 |
| 1005 | 16124067 | 201 | ---G--------------LF-----------------------------F---------- | 204 |
| 1006 | 16080147 | 183 | ---E--------------MD-----------------------------HFHYERLECNG | 196 |
| 1007 | 16273270 | 201 | ---G--------------LF-----------------------------Y---------- | 204 |
| 1008 | 15602409 | 201 | ---G--------------LY-----------------------------Y---------- | 204 |
| 1009 | 15672681 | 183 | ---A--------------LSELFGMGMERYFEGVVRHNGMLNMLKTGILY---------- | 215 |
| 1010 | 17938870 | 194 | ---AGMLGLPSHLCTVDCLE-----------------------------Y---------- | 211 |
| 1011 | 16119514 | 173 | ---AGMLGLPSHLCTVDCLE-----------------------------Y---------- | 190 |
| 1012 | 15613648 | 198 | ---A--------------LV-----------------------------H---------- | 201 |
| 1013 | 16331219 | 183 | ---A--------------IQ-----------------------------F---------- | 186 |
| 1013 | 17366711 | 178 | ---A--------------LSELFGMGMERYFEGVVRHNGMLNMLKTGILY---------- | 210 |
| 1014 | 15805621 | 215 | ---AG-------------LN-----------------------------F---------- | 219 |
| 1015 | 15620537 | 183 | ---A--------------LL-----------------------------Y---------- | 186 |
| 1016 | 15641730 | 208 | ---G--------------IA-----------------------------F---------- | 211 |
| 1017 | 7489711 | 418 | ---G--------------LK-----------------------------T---------- | 421 |
| 1018 | 15605532 | 199 | ---G--------------AL-----------------------------Y---------- | 202 |
| 1019 | 15803938 | 201 | ---G--------------LY-----------------------------Y---------- | 204 |
| 1020 | 17367076 | 187 | ---AG-------------LN-----------------------------F---------- | 191 |
| 1021 | 16766821 | 173 | ---H--------------MDDIELPWSFFNMHGLEFNGQLSFLKAGLY-Y---------- | 204 |
| 1022 | 16762766 | 173 | ---H--------------MDDIELPWSFFNMHGLEFNGQLSFLKAGLY-Y---------- | 204 |
| 1023 | 15834801 | 199 | ---G--------------AL-----------------------------Y---------- | 202 |
| 1024 | 15966599 | 204 | ---G--------------LQ-----------------------------T---------- | 207 |
| 1025 | 15618857 | 198 | ---G--------------AL-----------------------------Y---------- | 201 |
| 1026 | 17366350 | 181 | ---G--------------IV-----------------------------F---------- | 184 |
| 1027 | 15384987 | 357 | ---G--------------LK-----------------------------M---------- | 360 |
| 1028 | 2129898 | 472 | ---G--------------LK-----------------------------T---------- | 475 |
| 1029 | 2833384 | 472 | ---G--------------LK-----------------------------T---------- | 475 |
| 1030 | 15028467 | 414 | ---G--------------LK-----------------------------M---------- | 417 |
| 1031 | 15643657 | 209 | ---G--------------IV-----------------------------F---------- | 212 |
| 983 | Maizedu1 | 199 | -----------C--------------DK-------------A-----------------T | 203 |
| 984 | 7489826 | 1399 | -----------C--------------DK-------------A-----------------T | 1403 |
| 985 | 9502145 | 1336 | -----------C--------------DK-------------A-----------------T | 1340 |
| 986 | 9502143 | 1353 | -----------C--------------DK-------------A-----------------T | 1357 |
| 987 | 17646328 | 941 | -----------C--------------DK-------------A-----------------T | 945 |
| 988 | 4582789 | 871 | -----------A--------------DK-------------A-----------------T | 875 |
| 989 | 7489274 | 954 | -----------A--------------DK-------------A-----------------T | 958 |
| 990 | 2833389 | 954 | -----------A--------------DK-------------A-----------------T | 958 |
| 991 | 15221083 | 751 | -----------A--------------DK-------------A-----------------T | 755 |
| 992 | 4582783 | 590 | -----------S--------------NI-------------V-----------------T | 594 |
| 993 | 15717885 | 633 | -----------S--------------NI-------------V-----------------T | 637 |
| 994 | 15236819 | 758 | -----------S--------------NI-------------V-----------------T | 762 |
| 995 | 18461221 | 517 | -----------CGLDVEQLDRPDRMQDN-------------AHGRINVAKGGIVYSNIVT | 552 |
| 996 | 8901183 | 375 | -----------C--------------NA-------------V-----------------T | 379 |
| 997 | 17227527 | 215 | -----------S--------------NA-------------V-----------------T | 219 |
| 998 | 16329217 | 208 | -----------M--------------KG-------------G-----------------I | 212 |
| 999 | 2811062 | 208 | -----------S--------------DL-------------I-----------------T | 212 |
| 1000 | 7484399 | 165 | -----------C--------------NA-------------V-----------------T | 169 |
| 1001 | 18309046 | 214 | -----------S--------------DR-------------I-----------------L | 218 |
| 1002 | 15903076 | 208 | -----------A--------------NR-------------V-----------------S | 212 |
| 1003 | 17229371 | 187 | -----------A--------------DR-------------V-----------------N | 191 |
| 1004 | 15900991 | 208 | -----------A--------------NR-------------V-----------------S | 212 |
| 1005 | 16124067 | 205 | -----------A--------------DH-------------V-----------------T | 209 |
| 1006 | 16080147 | 197 | FVNFMKAGIIAA--------------DH-------------V-----------------T | 212 |
| 1007 | 16273270 | 205 | -----------S--------------DA-------------S-----------------T | 209 |
| 1008 | 15602409 | 205 | -----------S--------------DA-------------V-----------------T | 209 |
| 1009 | 15672681 | 216 | -----------A--------------DR-------------V-----------------N | 220 |
| 1010 | 17938870 | 212 | -----------Y--------------DDMSFLKGGLTTASAV-----------------T | 229 |
| 1011 | 16119514 | 191 | -----------Y--------------DDMSFLKGGLTTASAV-----------------T | 208 |
| 1012 | 15613648 | 202 | -----------S--------------DR-------------V-----------------T | 206 |
| 1013 | 16331219 | 187 | -----------A--------------NT-------------V-----------------T | 191 |
| 1014 | 17366711 | 211 | -----------A--------------DR-------------V-----------------N | 215 |
| 1015 | 15805621 | 220 | -----------A--------------GH-------------V-----------------T | 224 |
| 1016 | 15620537 | 187 | -----------A--------------DR-------------V-----------------N | 191 |
| 1017 | 15641730 | 212 | -----------A--------------DK-------------V-----------------N | 216 |
| 1018 | 7489711 | 422 | -----------A--------------DR-------------V-----------------V | 426 |
| 1019 | 15605532 | 203 | -----------CS-------------DF-------------V-----------------T | 208 |
| 1020 | 15803938 | 205 | -----------A--------------DH-------------I-----------------T | 209 |
| 1021 | 17367076 | 192 | -----------A--------------GH-------------V-----------------T | 196 |
| 1022 | 16766821 | 205 | -----------A--------------DH-------------I-----------------T | 209 |
| 1023 | 16762766 | 205 | -----------A--------------DH-------------I-----------------T | 209 |
| 1024 | 15834801 | 203 | -----------CS-------------DF-------------V-----------------T | 208 |
| 1025 | 15966599 | 208 | -----------A--------------SA-------------I-----------------T | 212 |
| 1026 | 15618857 | 202 | -----------CS-------------DY-------------I-----------------T | 207 |
| 1027 | 17366350 | 185 | -----------A--------------RR-------------V-----------------T | 189 |
| 1028 | 15384987 | 361 | -----------A--------------DR-------------A-----------------V | 365 |
| 1029 | 2129898 | 476 | -----------A--------------DR-------------I-----------------V | 480 |
| 1030 | 2833384 | 476 | -----------A--------------DR-------------I-----------------V | 480 |
| 1031 | 15028467 | 418 | -----------A--------------DR-------------A-----------------V | 422 |
| 1032 | 15643657 | 213 | -----------S--------------DV-------------I-----------------N | 217 |
| 983 | maize DU1 | 204 | TVSNTYSKE----V-------S--------G-------H-------G-----A------- | 218 |
| 984 | 7489826 | 1404 | TVSNTYSKE----V-------S--------G-------H-------G-----A------- | 1418 |
| 985 | 9502145 | 1341 | TVSPTYSRD----V-------A--------G-------H-------G-----A------- | 1355 |
| 986 | 9502143 | 1358 | TVSPTYSRD----V-------A--------G-------H-------G-----A------- | 1372 |
| 987 | 17646328 | 946 | TVSYTYSRE----V-------S--------G-------H-------G-----A------- | 960 |
| 988 | 4582789 | 876 | TVSPTYSRE----I-------A--------G-------N-------H-----A------- | 890 |
| 989 | 7489274 | 959 | TVSPTYSQE----V-------S--------G-------N-------P-----V------- | 973 |
| 990 | 2833389 | 959 | TVSPTYSQE----V-------S--------G-------N-------P-----V------- | 973 |
| 991 | 15221083 | 756 | TVSPTYAKE----V-------A--------G-------N-------S-----V------- | 770 |
| 992 | 4582783 | 595 | TVSPTYAQE----V-------R--------TAEGGKGLH-------S-----T------- | 616 |
| 993 | 15717885 | 638 | TVSPTYALE----V-------R--------S-------E-------G-----GRGLQDT- | 658 |
| 994 | 15236819 | 763 | TVSPTYAQE----V-------R--------TAEGGKGLH-------S-----T------- | 784 |
| 995 | 18461221 | 553 | TVSPTYALE----V-------R--------S-------E-------G-----GRGLQDT- | 573 |
| 996 | 8901183 | 380 | TVSPTYANE----V-------L--------N-------G-------G-----AAGWLRST | 401 |
| 997 | 17227527 | 220 | TVSPNHALE----A-------QYTDVGCGLG-------H-------------T------- | 241 |
| 998 | 16329217 | 213 | VYSN-YVNT----V-------S--------P-------H-------H-----AWEARFSD | 233 |
| 999 | 2811062 | 213 | TVSPTYKEE----I-------Q--------TAYYGERLD-------G-----L------- | 234 |
| 1000 | 7484399 | 170 | TVSPTYANE----V-------L--------N-------G-------G-----AAGWLRST | 191 |
| 1001 | 18309046 | 219 | TVSETYAKE----I---------------------------------------------- | 229 |
| 1002 | 15903076 | 213 | TVSPSYAHE----IMT-----S--------Q-------F-------G-----C------- | 229 |
| 1003 | 17229371 | 192 | TVSPTYAEQ----I-------KTP------A-------Y-------G-----E------- | 208 |
| 1004 | 15900991 | 213 | TVSPSYAHE----IMT-----S--------Q-------F-------G-----C------- | 229 |
| 1005 | 16124067 | 210 | TVSPTYAKE----I-------TQPAF----G-------Y-------GMEGLLQ------- | 233 |
| 1006 | 16080147 | 213 | TVSPTYRNE----I---------------------------------------------- | 222 |
| 1007 | 16273270 | 210 | AVSPTYAQE----I-------TTPEFAY--G-------L-------Q-----G------- | 230 |
| 1008 | 15602409 | 210 | AVSPTYAKE----I-------TTPEF----G-------Y-------GLQGLLT------- | 233 |
| 1009 | 15672681 | 221 | TVSPTYAKE----I-------QTSEFGC--G-------L-------E-----S------- | 241 |
| 1010 | 17938870 | 230 | TVSPTYARE----ILTPEMGMG--------M-------H-------G-----V------- | 251 |
| 1011 | 16119514 | 209 | TVSPTYARE----ILTPEMGMG--------M-------H-------G-----V------- | 230 |
| 1012 | 15613648 | 207 | TVSPTYAQE----I-------Q--------TPAFGEGLH-------G-----L------- | 228 |
| 1013 | 16331219 | 192 | TVSPTYAQQ----I-------QTP------A-------Y-------G-----E------- | 208 |
| 1014 | 17366711 | 216 | TVSPTYAKE----I-------QTSEFGC--G-------L-------E-----S------- | 236 |
| 1015 | 15805621 | 225 | TVSPTYAQE----I-------TTPQY----G-------E-------GLEGLLV------- | 248 |
| 1016 | 15620537 | 192 | TVSPTYAQQ----I-------QTP------T-------Y-------G-----E------- | 208 |
| 1017 | 15641730 | 217 | AVSPNYAAELLTPL-------G--------A-------H-------G-----L------- | 235 |
| 1018 | 7489711 | 427 | TVSNGYMWE----L-------K--------TSEGGWGLH-------D-----I------- | 448 |
| 1019 | 15605532 | 209 | TVSPTYAKE----ILEDYSDYE--------I-------H-------D-----A------- | 230 |
| 1020 | 15803938 | 210 | AVSPTYARE----I-------TEPQFAY--G-------M-------E-----G------- | 230 |
| 1021 | 17367076 | 197 | TVSPTYAQE----I-------TTPQY----G-------E-------GLEGLLV------- | 220 |
| 1022 | 16766821 | 210 | AVSPTYARE----I-------TEPQFAY--G-------M-------E-----G------- | 230 |
| 1023 | 16762766 | 210 | AVSPTYARE----I-------TEPQFAY--G-------M-------E-----G------- | 230 |
| 1024 | 15834801 | 209 | TVSPTYAKE----ILQDYSDYE--------I-------H-------D-----A------- | 230 |
| 1025 | 15966599 | 213 | TVSPSYAQE----ILTPEFGMG--------L-------D-------G-----L------- | 234 |
| 1026 | 15618857 | 208 | TVSLTYVQE----I-------I--------NDYSDYELH-------D-----A------- | 229 |
| 1027 | 17366350 | 190 | TVRPSYAEE----I-------Q--------TPEFGMGLD-------G-----V------- | 211 |
| 1028 | 15384987 | 366 | TVSHCYLWE----I-------KTMDGGW--G-------L-------H-----E------- | 386 |
| 1029 | 2129898 | 481 | TVSHCYAWE----L-------K--------TSEGGWGLH-------N-----I------- | 502 |
| 1030 | 2833384 | 481 | TVSHCYAWE----L-------K--------TSEGGWGLH-------N-----I------- | 502 |
| 1031 | 15028467 | 423 | TVSHGYLWE----I-------KTMDGGW--G-------L-------H-----E------- | 443 |
| 1032 | 15643657 | 218 | TVSPTYAEE----I-------Q--------T-------EEYGEKLDG-----V------- | 239 |
| 983 | maize Du1 | 219 | --------I---V--PHL-----G-K------------FYGILNGIDPDIWDP------Y | 241 |
| 984 | 7489826 | 1419 | --------I---V--PHL-----C-K------------FYGILNGIDPDIWDP------Y | 1441 |
| 985 | 9502145 | 1356 | --------I---A--PHR-----E-K------------FYGILNGIDPDIWDP------Y | 1378 |
| 986 | 9502143 | 1373 | --------I---A--PHR-----F-K------------FYGILNGIDPDIWDP------Y | 1395 |
| 987 | 17646328 | 961 | --------I---A--PHF-----S-K------------FHGIRNGIDPDIWDP------Y | 983 |
| 988 | 4582789 | 891 | --------V---A--THL-----H-K------------FHGIINGIDPDIWDP------F | 913 |
| 989 | 7489274 | 974 | --------I---A--PHL-----H-K------------FHGIVNGIDPDIWDP------L | 996 |
| 990 | 2833389 | 974 | --------I---A--PHL-----H-K------------FHGIVNGIDPDIWDP------L | 996 |
| 991 | 15221083 | 771 | --------I---S--AHL-----Y-K------------FHGIINGIDPDIWDP------Y | 793 |
| 1033 | 7489827 | 1 | DP------Y | 3 |
| 992 | 4582783 | 617 | --------L---S--THS-----K-K------------FIGILNGIDTDIWNP------A | 639 |
| 993 | 15717885 | 659 | --------L---K--VHS-----R-K------------FLGILNGIDTDTWNP------C | 681 |
| 994 | 15236819 | 785 | --------L---N--FHS-----K-K------------FIGILNGIDTDSWNP------A | 807 |
| 995 | 18461221 | 574 | --------L---K--MHS-----R-K------------FVGILNGIDTGTWNP------S | 596 |
| 996 | 8901183 | 402 | F-------A---R--PELR----S-K------------FHGILNGIDCEEWNP------A | 426 |
| 997 | 17227527 | 242 | --------L---Y--LHK-----E-K------------FSGVLNGIDYDFWNP------E | 264 |
| 998 | 16329217 | 234 | ISCGLGHTL---E--IHQ-----Q-K------------FGGILNGLDYEVWNP------E | 264 |
| 999 | 2811062 | 235 | --------L---R--ARR-----D-D------------LLGILNGIDDEFYNP------E | 257 |
| 1000 | 7484399 | 192 | F-------A---R--PELR----S-K------------FHGILNGIDCEEWNP------A | 216 |
| 1001 | 18309046 | 230 | ------------T--PYF-----G-ENLDGLLRERGYALKGIVNGIDYDEFNP------S | 263 |
| 1002 | 15903076 | 230 | --------N---L--DHILKMESG-K------------VSGIVNGIDADLYNP------Q | 257 |
| 1003 | 17229371 | 209 | --------K---I--EGLLSFISG-K------------LSGIVNGIDTEVYDP------A | 236 |
| 1004 | 15900991 | 230 | --------N---L--DQILKMESG-K------------VSGIVNGIDADLYNP------Q | 257 |
| 1005 | 16124067 | 234 | --------A---L--ARQ-----G-R------------LTGILNGVDSDIWDP------Q | 256 |
| 1006 | 16080147 | 223 | --------M---T--PYY-----G-EQLEQVLQYREDDVTGILNGIDDTFYQP------K | 257 |
| 1007 | 16273270 | 231 | --------L---L--SGLKAQ--G-R------------LVGILNGVDENIWHP------N | 256 |
| 1008 | 15602409 | 234 | --------T---L--NSQ-----G-K------------LVGILNGVDDQIWHP------N | 256 |
| 1009 | 15672681 | 242 | --------I---L--QYVD----G-K------------VSGILNGIDYDIYNP------E | 265 |
| 1010 | 17938870 | 252 | --------L---A--RRR-----G-D------------LRGIVNGVDHDVWNP------A | 274 |
| 1011 | 16119514 | 231 | --------L---A--RRR-----G-D------------LRGIVNGVDHDVWNP------A | 253 |
| 1012 | 15613648 | 229 | --------L---H--QER-----G-K------------TRGILNGIDLEDFDP------K | 251 |
| 1013 | 16331219 | 209 | --------K---L--EGLLSYLSG-N------------LVGILNGIDTEIYNP------A | 236 |
| 1014 | 17366711 | 237 | --------I---L--QYVD----G-K------------VSGILNGIDYDIYNP------E | 260 |
| 1015 | 15805621 | 249 | --------R---L--THE-----G-R------------LSGILNGLDQDRWNP------R | 271 |
| 1016 | 15620537 | 209 | --------K---L--EGLLSFISG-K------------LSGILNGIDVDSYNP------A | 236 |
| 1017 | 15641730 | 236 | --------VDDFV--RRA-----R-D------------LHGIVNGCDYSEWNP------R | 261 |
| 1018 | 7489711 | 449 | --------I---N--QND-----W-K------------LQGIVNGIDMSEWNPAVDVHLH | 477 |
| 1019 | 15605532 | 231 | --------I---T--ARQ-----H-H------------LRGILNGIDTTIWGP------E | 253 |
| 1020 | 15803938 | 231 | --------L---L--QQRHRE--G-R------------LSGVLNGVDEKIWSP------E | 256 |
| 1021 | 17367076 | 221 | --------R---L--THE-----C-R------------LSGILNGLDQDRWNP------R | 243 |
| 1022 | 16766821 | 231 | --------L---LRQHHLE----G-R------------LSGILNGVDEKIWNF------E | 256 |
| 1023 | 16762766 | 231 | --------L---LHQRHLE----G-R------------LSGILNGVDEKIWNP------E | 256 |
| 1024 | 15834801 | 231 | --------I---T--ARQ-----H-H------------LKGILNGIDYTILDP------E | 253 |
| 1025 | 15966599 | 235 | --------L---S--SRV-----A-D------------LTGIVNGIDGETWDP------Q | 257 |
| 1026 | 15618857 | 230 | --------I---L--ARN-----S-V------------FSGIINGIDEDVWNP------K | 252 |
| 1027 | 17366350 | 212 | --------L---R--RHA-----G-K------------LRGILNGLDTEVFDP------G | 234 |
| 1028 | 15384987 | 387 | --------I---I--NHN-----DWK------------LQGIVNGIDMAEWNP------E | 410 |
| 1029 | 2129898 | 503 | --------I---N--ESD-----W-K------------FRGIVNGVDTKDWNP------Q | 525 |
| 1030 | 2833384 | 503 | --------I---N--ESD-----W-K------------FHGIVNGVDTKDWNP------Q | 525 |
| 1031 | 15028467 | 444 | --------I---I--NHN-----DWK------------LQGIVNGIDMAEWNP------E | 467 |
| 1032 | 15643657 | 240 | --------L---R--MHS-----K-D------------LYGILNGIDYELYNP------A | 262 |
| 983 | MaizeDu1 | 242 | NDNFIP----VHYT------CE------NVVEGKHAAKRA-LQQKFGL-Q-QI--D--V- | 277 |
| 984 | 7489826 | 1442 | NDNFIP----VHYT------CE------NVVEGKHAAKRA-LQQKFGL-Q-QI--D--V- | 1477 |
| 985 | 9502145 | 1379 | TDNFIP----VFYT------CE------NVVEGKPAAKHA-LQQKFGL-Q-QT--D--V- | 1414 |
| 986 | 9502143 | 1396 | TDNFIP----VPYT------CE------NVVEGKHAAKRA-LQQKFGL-Q-QT--D--V- | 1431 |
| 987 | 17646328 | 984 | SDNFIP----VHYT------SE------NVVEGKSAAKKA-LQQRLGL-Q-QT--D--T- | 1019 |
| 988 | 4582789 | 914 | NDNSIP----VPYT------AE------NVVEGKRASKEA-LQQKLGL-K-KA--D--L- | 949 |
| 989 | 7489274 | 997 | NDKFIP----IPYT------SE------NVVEGKTAAKEA-LQHKLGL-K-QA--D--L- | 1032 |
| 990 | 2833389 | 997 | NDKFIP----IPYT------SE------NVVEGKTAAKEA-LQHKLGL-K-QA--D--L- | 1032 |
| 991 | 15221083 | 794 | NDNFIF----VPYT------SE------NVVEGKHAAKEE-LQNHLGL-K-SA--D--F- | 829 |
| 1033 | 7489827 | 4 | NDNFIP----VHYT------CE------NVVEGKHAAKRA-LQQKFGL-Q-QI--D--V- | 39 |
| 992 | 4582783 | 640 | TDPFLQ----VQYN------A-------NDLQGKSENKEA-LRRNLGL-S-SA--D--VR | 675 |
| 993 | 15717885 | 682 | TDRYLK----VQYN------AK------D-LQGKAANKAA-LREQLNL-A-SAYPS--Q- | 718 |
| 994 | 15236819 | 808 | TDPFLK----AQFN------AK------D-LQGKEENKMA-LRKQLGL-s-SA--E--SR | 843 |
| 995 | 18461221 | 597 | TDRFLA----VQYS------AT------D-LQGKAANKAF-LRKQLGL-Y-SE--D--AS | 632 |
| 996 | 8901183 | 427 | TDALLP----ANFD------AD------RPA-GKALCKEF-LQKGLGL---EV--DPRK- | 462 |
| 997 | 17227527 | 265 | IDRHIP----DNYS-------Q------DDFEQKLYNKKA-LRERLLL-Q-AA--D--K- | 299 |
| 998 | 16329217 | 265 | IDPLLA----SNFS------VK------TFGD-KAKNKQA-LRERLLL-E-TD--DK-K- | 300 |
| 999 | 2811062 | 258 | ADPFLT----ATYS------V-------HTRERKQLNKRA-LQRQFGL-P-EWD-D--V- | 293 |
| 1000 | 7484399 | 217 | TDALLP----ANFD------AD------RPA-GKALCKEF-LQKGLGL---EV--DPRK- | 252 |
| 1001 | 18309046 | 264 | KDSLIA----KNFS------VK-------TIEDKVLNKLA-LQKELGL-PINP--D--I- | 299 |
| 1002 | 15903076 | 258 | TDALLD----YHFN------QE------D-LSGKAKNKAK-LQERVGL-PVRA--D--V- | 293 |
| 1003 | 17229371 | 237 | NDKYIA----QTFT------AD-------TLDKRKANKIA-LQEEVGL-EVNS--N--A- | 272 |
| 1004 | 15900991 | 258 | TDALLD----YHFN------QE------D-LSGKAKNKAK-LQERVGL-PVRA--D--V- | 293 |
| 1005 | 16124067 | 257 | SDTLLP----TRYD------AE------N-LQAKAINKTH-LQTAMGL-Q-LA--EN-K- | 292 |
| 1006 | 16080147 | 258 | SDPYIE----AQYD------SG------DLA-CKLENKTK-LQQRMGLPE-KN--D--I- | 293 |
| 1007 | 16273270 | 257 | VDQYIP----HHYK------LK-------YMAGKKKNKAE-LQAYFNL-P-QD--E--S- | 291 |
| 1008 | 15602409 | 257 | HDAYIE----HEYK------LK-------AMTGKRKNKEA-LQAYFNLPQ-DP--D--A- | 292 |
| 1009 | 15672681 | 266 | NDILIP----YHFS-------E------EELSGKGQMKAE-LQKRTGL-PLNP--N--V- | 301 |
| 1010 | 17938870 | 275 | TDPYIL----ANFT------AA------TATR-RSLNKYA-LLQALGL-A-PT--Q--G- | 309 |
| 1011 | 16119514 | 254 | TDPYIL----ANFT------AA------TATR-RSLNKYA-LLQALGL-A-PT--Q--G- | 288 |
| 1012 | 15613648 | 252 | TDPHVT----YPYK-------H------NQME-KRKNKQV-IQRLFELPE-RK--D--I- | 286 |
| 1013 | 16331219 | 237 | EDRFIS----NVFD------AD------S-LDKRVKNKIA-IQEETGL-EINR--N--A- | 272 |
| 1014 | 17366711 | 261 | NDILIP----YHFS-------E------EELSGKGQMKAE-LQKRTGL-PLNP--N--V- | 296 |
| 1015 | 15805621 | 272 | TDPDIA----AY----------------SDPAGKAGAVKA-LRQEFGL-D-----D--A- | 301 |
| 1016 | 15620537 | 237 | TDRGXV----ANYD------RD------TLI-ARLNNRLA-LQKEMGL-EVNP--D--R- | 272 |
| 1017 | 15641730 | 262 | TDHYLP----ATYSDE----PE------SMRKGKALCKTA-LQEELHL-P-VT--D--V- | 299 |
| 1018 | 7489711 | 478 | SDDY------TNYT------FE------TLDTGKRQCKAA-LQRQLGL-QVRD--D--V- | 512 |
| 1019 | 15605532 | 254 | TDPNLA----KNYT------KELFETPSIFFEAKAENKKA-LYERLGL-S-LE--H--S- | 295 |
| 1020 | 15803938 | 257 | TDLLLA----SRYT-------R------DTLEDKAENKRQ-LQIAMGL-K-VD--DK-V- | 292 |
| 1021 | 17367076 | 244 | TDPDIA----AY----------------SDPAGKAGAVKA-LRQEFGL-D-----D--A- | 273 |
| 1022 | 16766821 | 257 | SDLLLA----SRYT-------R------DTLEEKAENKRQ-LQIAMGL-K-VN--DK-V- | 292 |
| 1023 | 16762766 | 257 | SDLLLA----SRYT-------R------DTLEEKAENKRQ-LQIAMGL-K-VN--DK-V- | 292 |
| 1024 | 15834801 | 254 | TDPHLA----KNYSKVLFEDPK------AFFEAKAENKKA-LYETLGL-S-LD--K--S- | 295 |
| 1025 | 15966599 | 258 | TDPHIP----AHYG-------P------GTLKRRAGNRKA-LEERFGL-E-KG--P--G- | 292 |
| 1026 | 15618857 | 253 | TDPALA----VQYD------ASLLSEP-DVLFTKKEENRAVLYEKLGI-S-SD--Y--F- | 294 |
| 1027 | 17366350 | 235 | KDPYLP----APYT------RE------D-PSGKARAKEV-FRERTGL-R-P-------- | 266 |
| 1028 | 15384987 | 411 | VDEHLQSDGYANYT------FE------TLDTGKKQCKEA-LQRQLGL-QVRD--D--V- | 451 |
| 1029 | 2129898 | 526 | FDAYLTSDGYTNYN------LK------TLQTGKRQCKAA-LQRELGL-PVRE--D--V- | 566 |
| 1030 | 2833384 | 526 | FDAYLTSDGYTNYN------LK------TLQTGKRQCKAA-LQRELGL-PVRE--D--V- | 566 |
| 1031 | 15028467 | 468 | VDEHLQSDGYANYT------FE------TLDTGKKQCKEA-LQRQLGL-QVRD--D--V- | 508 |
| 1032 | 15643657 | 263 | TDRYIY----VNYD------V-------NRLELKWENKVK-LQEELGL-PVNK--E--T- | 298 |

TABLE XXXIV
Maize soluble starch synthase III (Du I)
“GLYTR” Domain Alignments with other similar proteins

| SEQ | Accession | a.a | | a.a. |
|---|---|---|---|---|
| Id.No. | Number | # | (start) Sequence end | # |
| 1034 | MaizeDu1 | 278 | -PVVGI-VTRLTAQKGIHLIKH-AIHRTLE-RNG-QVVLLGS-A-PDSRIQADFVNLANT | 330 |
| 1035 | 7489826 | 1478 | -PVVGI-VTRLTAQKGIHLIKH-AIHRTLE-RNG-QVVLLGS-A-PDSRIQADFVNLANT | 1530 |
| 1036 | 9502145 | 1415 | -PIVGI-ITRLTAQKGIHLIKH-AIHRTLE-SNG-QVVLLGS-A-PDHRIQGDFCRLADA | 1467 |
| 1037 | 9502143 | 1432 | -PIVGI-ITRLTAQKGIHLIKH-AIHRTLE-SNG-HVVLLGS-A-PDHRIQGDFCRLADA | 1484 |
| 1038 | 17646328 | 1020 | -PVVGI-ISRLTVQKGIHLIKH-AIYRTLE-RNG-QVVLLGS-A-PDHRIQGDFTNLASK | 1072 |
| 1039 | 4582789 | 950 | -PLVGV-ITRLTHQKGIHLIKH-AIWRTLE-RGG-QVVLLGS-A-PDHRIQNDFVNLANQ | 1002 |
| 1040 | 7489274 | 1033 | -PLVGI-ITRLTHQKGIHLIKH-AIWRTLE-RNG-QVVLLGS-A-PDPRVQNDFVNLANQ | 1085 |
| 1041 | 2833389 | 1033 | -PLVGI-ITRLTHQKGIHLIKH-AIWRTLE-RNG-QVVLLGS-A-PDPRVQNNFVNLANQ | 1085 |
| 1042 | 15221083 | 830 | -PVVGI-ITRLTHQKGIHLIKH-AIWRTLE-RNG-QVVLLGS-A-PDPRIQNDFVNLANQ | 882 |
| 1043 | 7489827 | 40 | -PVVGI-VTRLTAQKGIHLIKH-AIHRTLE-RNG-QVVLLGS-A-PDARIQADFVNLANK | 92 |
| 1044 | 4582783 | 676 | RPLVGC-ITRLVPQKGVHLIRH-AIYLTLE-LGG-QFVLLGS-S-PVPHIQREFEGIAN- | 728 |
| 1045 | 15717885 | 719 | -PLVGC-ITRLVAQKGVHLIRH-AIYKTAE-LGG-QFVLLGS-S-PVPEIQREFEGIAD- | 770 |
| 1046 | 15236819 | 844 | RPLVGC-ITRLVPQKGVHLIRH-AIYRTLE-LGG-QFVLLGS-S-PVPHIQREFEGIEQQ | 897 |
| 1047 | 18461221 | 633 | QPLVAC-ITRLVPQKGLHLIRH-AIYKTAE-LGG-QFVLLGS-S-PVPHIQREFEGVADQ | 686 |
| 1048 | 8901183 | 463 | -PLVAV-VSRLVPQKGIHLIKA-ALFRTVE-KGG-QFVLLGS-G-HSD---PAFRQLADQ | 512 |
| 1049 | 17227527 | 300 | -PIIAY-IGRLDNQKGVHLVHH-AIYHALN-KGA-QFVLLGS-A-TEAGINAHFRHEKQF | 352 |
| 1050 | 16329217 | 301 | -PMLCF-IGRLDGQKGVHLVHH-SIYYALS-QGA-QFVLLGS-A-TEPNLSKWFWHEKQH | 353 |
| 1051 | 2811062 | 294 | -PLIAM-VTRMTAQKGLDLVTC-VFHEMMS-EDM-QLVVLGT-G--DWRFEQFFSQMA-- | 343 |
| 1052 | 7484399 | 253 | -PLVAV-VSRLVPQKGIHLIKA-ALFRTVE-KGG-QFVLLGS-G-HSD---PAFRQLADG | 302 |
| 1053 | 18309046 | 300 | -PMISI-VSRLTNQKGCDLIVN-IANRLLQ-RNV-QLVILGT-G------DYNYENHFKG | 347 |
| 1054 | 15903076 | 294 | -PLVGI-VSRLTRQKGFDVVVE-SLHHILQ-EDV-QIVLLGT-G--DPAFEGAFSWFAQ- | 344 |
| 1055 | 17229371 | 273 | -FLIGM-VTRLVEQKGLDLVIQ-MLDRFMAYTDA-QFVLLGT-G--DRYYETQMWQLASR | 325 |
| 1056 | 15900991 | 294 | -PLVGI-VSRLTRQKGFDVVVE-SLHHILQ-EDV-QIVLLGT-G--DPAFEGAFSWFAQ- | 344 |
| 1057 | 16124067 | 293 | -PIFAV-VSRLTVQKGLDLVLE-ALPELLA-LGG-QLVVLGS-G--DATLQEAFLAAA-- | 342 |
| 1058 | 16080147 | 294 | -PLISM-VTRLTKQKGLDLVRR-IMHELLE-EQDIQLVVLGT-G------EREFEDYFRY | 342 |
| 1059 | 16273270 | 292 | -ALAFVMVTRLTEQKGVDLLIE-SADEIVK-QGG-QLMILGSGA-P--HLEQGIRELAER | 344 |
| 1060 | 15602409 | 293 | -LLFVM-VTRLTEQKGVDLLID-SAEEIVK-QGG-QLTILGS-G--SPHLEAGILHLAQQ | 344 |
| 1061 | 15672681 | 302 | -PLIGM-VSRLTNQKGFDLVLS-QLEKVLE-ENV-QIVLLGT-GFPE--IEEGFRYFSQK | 353 |
| 1062 | 17938870 | 310 | -PVFGV-VSRLTWQKGIDLLPH-VVPLIIE-RKG-RLIVHGE-G--DTALEDSLQALAKR | 361 |
| 1063 | 16119514 | 289 | -PVFGV-VSRLTWQKGIDLLPH-VVPLIIE-RKG-RLIVHGE-G--DTALEDSLQALAKR | 340 |
| 1064 | 15613648 | 287 | -PLIAM-VSRLVEEKGVPLLTQIAGELVTT-ENV-QLAILGT-G--DPSLEDQLHHLA-S | 338 |
| 1065 | 16331219 | 273 | -MVVGI-VARLVEQKGIDLVIQ-ILDRFMSYTDS-QLIILGT-G--DRHYETQLWQMASR | 325 |
| 1066 | 17366711 | 297 | -PLIGM-VSRLTNQKGFDLVLS-QLEKVLE-ENV-QIVLLGT-GFPE--IEEGFRYFSQK | 348 |
| 1067 | 15805621 | 302 | -PILAT-VSRLADQKGMDLLIT-ALPE-LV-RDW-NVVVLGG-G--DPLLEAALTGWA-- | 350 |
| 1068 | 15620537 | 273 | -FLIGF-VARLVEQKGIDLLLQ-ILDRFLSYSDA-QFVVLGT-G--ERYYETQLWELATR | 325 |
| 1069 | 15641730 | 300 | -PLFGM-VCRLTHQKGFHYLLP-ILEQFLR-NNV-QVVIVGT-G------EPEVAARLNK | 347 |
| 1070 | 7489711 | 513 | -PLIGF-IGRLDHQKGVDIIAD-AIHWIAG-QDV-QLVMLGT-G------RADLEDMLRR | 560 |
| 1071 | 15605532 | 296 | -PCVCI-ISRIAEQKGPHFMKQ-AILHALE-NAY-TLIIIGT-C-YGNQLHEEFANLQES | 348 |
| 1072 | 15803938 | 293 | -PLFAV-VSRLTSQKGLDLVLE-ALPGLLE-QGG-GLALLG--A-GDPVLQEGFLAAAAE | 344 |
| 1073 | 17367076 | 274 | -PILAT-VSRLADQKGMDLLIT-ALPE-LV-RDW-NVVVLGG-G--DPLLEAALTGWA-- | 322 |
| 1074 | 16766821 | 293 | -PLFAV-VSRLTNQKGLDLVLE-ALPGLLE-QGG-QLALLG--A-GDPVLQEGFLAAA-- | 342 |
| 1075 | 16762766 | 293 | -PLFAV-VSRLTNQKGLDLVLE-ALPGLLE-QGG-QLALLG--A-GDPVLQEGFLAAA-- | 342 |
| 1076 | 15834801 | 296 | -PCMCI-ISRIAEQKGPEFMKQ-AILHALE-NAY-TLIIIGT-C-YGGQIHKEFSNLQES | 348 |
| 1077 | 15966599 | 293 | -PIFCV-ISRLTWQKGMDLVAE-AADDIVA-LGG-KLVVLGS-G--DPALESALMAAASR | 344 |
| 1078 | 15618857 | 295 | -PLICV-ISRIVEEKGPEFMKE-IILHAME-HSY-AFILIGT-S-QNEVLLNEFRNLQDC | 347 |
| 1079 | 17366350 | 267 | -PVLAY-VGRLDYQKGLDLVLK-ALPRLLE-MGF-RLYVQGV-G--DGGLQEAFLRAEEE | 318 |
| 1080 | 15384987 | 452 | -PLIGF-IGRLDHQKGVDIIGD-AMPWIAG-QDV-QVVMLGT-G------RPDLEEMLRR | 499 |
| 1081 | 2129898 | 567 | -PIISF-IGRLDHQKGVDLIAE-AIPWMMS-HDV-QLVMLGT-G------RADLEQMLKE | 614 |
| 1082 | 2833384 | 567 | -PIISF-IGRLDHQKGVDLIAE-AIPWMMS-HDV-QLVMLGT-G------RADLEQMLKE | 614 |
| 1083 | 15028467 | 509 | -PLIGF-IGRLDHQKGVDIIGD-AMPWIAG-QDV-QVVMLGT-G------RPDLEEMLRR | 556 |
| 1084 | 15643657 | 299 | -AVAGL-ISRLVPQKGLDLLVD-VMDYLTL-FDL-QIVVLGT-G-DEQ-----YENAFRK | 346 |
| 1034 | Maize DU1 | 331 | LHGVNHGQVRLSLTYDEPLSHLIYAGSDFILVPSIFEPCGLTQLVAMRYGTIPIVRKTGG | 390 |
| 1035 | 7489826 | 1531 | LHGVNHGQVRLSLTYDEPLSHLIYAGSDFILVPSIFEPCGLTQLVAMRYGTIPIVRKTGG | 1590 |
| 1036 | 9502145 | 1468 | LHGVYHGRVKLVLTYDEPLSHLIYAGSDFIIVPSIFEPCGLTQLVAMRYGSIPIVRKTGG | 1527 |
| 1037 | 9502143 | 1485 | LHGVYHGRVKLVLTYDEPLSHLIYAGSDFIIVPSIFEPCGLTQLVAMRYGSIPIVRKTGG | 1544 |
| 1038 | 17646328 | 1073 | LHGEYHGRVKLCLTYDEPLSHLIYAGADFILVPSMFEPCGLTQLTAMRYGSIPIVRKTGG | 1132 |
| 1039 | 4582789 | 1003 | LHSSHNDRARLCLAYDEPLSHMIYAGADFILVPSIFEPCGLTQLTAMRYGSIPIVRKTGG | 1062 |
| 1040 | 7489274 | 1086 | LHSKYNDRARLCLTYDEPLSHLIYAGADFILVPSIFEPCGLTQLTAMRYGSIPVVRKTGG | 1145 |
| 1041 | 2833389 | 1086 | LHSKYNDRARLCLTYDEPLSHLIYAGADFILVPSIFEPCGLTQLTAMRYGSIPVVRKTGG | 1145 |
| 1042 | 15221083 | 883 | LHSSHGDRARLVLTYDEPLSHLIYAGADFILVPSIFEPCGLTQLIAMRYGAVPVVRKTGG | 942 |
| 1043 | 7489827 | 93 | LHGVNHGQCRLSLTYDEPLSHLIYAGSDFILVPSIFEPCGLTHLVAMRYGTIPIVRKTGG | 152 |
| 1044 | 4582783 | 729 | -HFQNHDHIRLILKYDESLSHAIYAASDMFIIPSIFEPCGLTQMISMRYGAIPIARKTGG | 787 |
| 1045 | 15717885 | 771 | -HFQNNNNIRLILKYDDALSHCIYAASDMFIVPSIFEPCGLTQMIAMRYGSVPIVRKTGG | 829 |
| 1046 | 15236819 | 898 | FK--SHDHVRLLLKYDEALSHTIYAASDLFIIPSIFEPCGLTQMIAMRYGSIPIARKTGG | 955 |
| 1047 | 18461221 | 687 | FQKNNN--IRLILKYDEALSHCIYAASDMFIIPSMFEPCGLTQMIAMRYGSVPIVRQTGG | 744 |
| 1048 | 8901183 | 513 | QFK-DHPNCRLKIMYSERLAHMIYAAADVVVVPSMFEPCGLTQMIALRYGAVPLVRRTGG | 571 |
| 1049 | 17227527 | 353 | LN--SNPDVHLELGFNEELSHLIYAGADMIVVPSNYEPCGLTQMIGLKYGTVPIVRGVGG | 410 |
| 1050 | 16329217 | 354 | LN--DNPNVHLELGFDEELAHLIYGAADIIVVPSNYEPCGLTQMIGLRYGAVPVVRGVGG | 411 |
| 1051 | 2811062 | 344 | --AAYPGKVGVYIGFHEPLAHQIYAGADLFLIPSLFEPCGLSQMIALRYGTIPIVRETGG | 401 |
| 1052 | 7484399 | 303 | QFK-DHPNCRLKIMYSERLAHMIYAAADVVVVPSMFEPCGLTQMIALRYGAVPLVRRTGG | 361 |
| 1053 | 18309046 | 348 | LQELYPTKVSANIKFDNGLAHRIYASSDIFLMPSLFEPCGLGQLIALRYGAIPIVRETGG | 407 |
| 1054 | 15903076 | 345 | ---IYPDKLSTNITFDVKLAQEIYAACDLFLMPSRFEPCGLSQMMAMRYGTLPLVHEVGG | 401 |
| 1055 | 17229371 | 326 | Y----PGRMATYLLYNDALSRRIYAGTDAFLMPSRFEPCGISQMMALRYGSIPIVRRTGG | 381 |
| 1056 | 15900991 | 345 | ---IYPDKLSTNITFDVKLAQEIYAACDLFLMPSRFEPCGLSQMMAMRYGTLPLVHEVGG | 401 |
| 1057 | 16124067 | 343 | --AEHSGQVGVQIGYHEAFSHRIIAGSDVILVPSRFEPCGLTQLYGLKYGTLPLVRHTGG | 400 |
| 1058 | 16080147 | 343 | AEFAFHEKCRAYIGFDEPLAHQIYAGSDMFLMPSKFEPCGLGQLIALQYGAIPIVRETGG | 402 |
| 1059 | 16273270 | 345 | Y----PQNIAVKIGYDEALSHLMVAGGDVILVPSRFEPCGLTQLYGLQYGTLPLVRKTGG | 400 |
| 1060 | 15602409 | 345 | Y----PHQIAVKIGYDEALSHLMIAGGDVILVPSRFEPCGLTQLYGLKYGTLPLVRATGG | 400 |
| 1061 | 15672681 | 354 | Y----PDKLSANIAFDIQFAQEIYAGSDFFLMPSAFEPCGLSQMIAMRYGTLPIVHEIGG | 409 |
| 1062 | 17938870 | 362 | YPEL----VCAHIGYDERLAHMIQAGSDFIIQPSRFEPCGLTQLYLARYGALPIVSRTGG | 417 |
| 1063 | 16119514 | 341 | YPEL----VCAHIGYDERLAHMIQAGSDFIIQPSRFEPCGLTQLYALRYGALPIVSRTGG | 396 |
| 1064 | 15613648 | 339 | LHP---HQISFKCVFAEPLARKLYAGADLFIMPSRFEPCGLSQMISLRYETVPIVRETGG | 395 |
| 1065 | 16331219 | 326 | F----PGRMAVQLLHNDALSRRVYAGADVFLMPSRFEPCGLSQLMAMRYGCIPIVRRTGG | 381 |
| 1066 | 17366711 | 349 | Y----PDKLSANIAFDIQFAQEIYAGSDFFLMPSAFEPCGLSQMIAMRYGTLPIVHEIGG | 404 |
| 1067 | 15805621 | 351 | ----NHPRVAFASGMNEPLAHRIYAGAHAFAMPSRFEPCGLSQLIAMRYGTLPIVRETGG | 406 |
| 1068 | 15620537 | 326 | Y----PGRMSTYLMYDEGLSRRIYAGSDAFLVPSRFEPCGITQMLALRYGSVPIVRRTGG | 381 |
| 1069 | 15641730 | 348 | IAHYHRAKFAFVETYSERLAHWVEAGSDFFLMPSEFEACGLNQIYSMAYGTLPIVREVGG | 407 |
| 1070 | 7489711 | 561 | FESEHSDKVRAWVGFSVPLAHRITAGADILLMPSRFEPCGLNQLYAMAYGTVPVVHAVGG | 620 |
| 1071 | 15605532 | 349 | L--ANSPDVRILLTYSDVLARQIFAAADMICIPSMFEPCGLTQMIGMRYGTVPLVRATGG | 406 |
| 1072 | 15803938 | 345 | Y----PGQVGVQIGYHEAFSHRIMGGADVILVPSRFEPCGLTQLYGLKYGTLPLVRRTGG | 400 |
| 1073 | 17367076 | 323 | ----NHPRVAFASGMNEPLAHRIYAGAHAFAMPSRFEPCGLSQLIAMRYGTLPIVRETGG | 378 |
| 1074 | 16766821 | 343 | --AEHPGQVGVQIGYHEAFSHRIMGGADVILVPSRFEPCGLTQLYGLKYGTLPLVRRTGG | 400 |
| 1075 | 16762766 | 343 | --AEHPGQAGVQIGYHEAFSHRIMGGADVILVPSRFEPCGLTQLYGLKYGTLPLVRRTGG | 400 |
| 1076 | 15834801 | 349 | L--ADSPNVRILLTYSDVLARQIFAAADMICIPSMFEPCGLTQMIGMRYGTVPVVRATGG | 406 |
| 1077 | 15966599 | 345 | ----NRGHIGMVTGYDEPLSHLMQAGADAILIPSRFEPCGLTQLYGLRYGCVPVVARTGG | 400 |
| 1078 | 15618857 | 348 | L--ASSPNIRLILDFNDPLARLTYAAADMICIPSHREACGLTQLIAMRYGTVPLVRKTGG | 405 |
| 1079 | 17366350 | 319 | ----NPEGVRFLPAYDEAMARLAYAGAEAVLVPSRFEPCGLVQMIASRYGTPPVARAVGG | 374 |
| 1080 | 15384987 | 500 | FESEHNDKVRGWVGFSVQLAHRITAGADVLLMPSRFEPCGLNQLYAMAYGTVPVVHAVGG | 559 |
| 1081 | 2129898 | 615 | FEAQHCDKIRSWVGFSVKMAHRITAGSDILLMPSRFEPCGLNQLYAMSYGTVPVVHGVGG | 674 |
| 1082 | 2833384 | 615 | FEAQHCDKIRSWVGFSVKMAHRITAGSDILLMPSRFEPCGLNQLYAMSYGTVPVVHGVGG | 674 |
| 1083 | 15028467 | 557 | FESEHNDKVRGWVGFSVQLAHRITAGADVLLMPSRFEPCGLNQLYAMAYSTVPVVHAVGG | 616 |
| 1084 | 15643657 | 347 | FQERYPDKVSANIKFDVELAQKIYAGADIFLMPSRYEPCGLGQMFSMRYGTIPVVRYTGG | 406 |
| 1034 | MaizeDu1 | 391 | LFDTV--FD--VDNDKERARDRGLE---P-----N-GFS-FDGA-DS------------- | 422 |
| 1035 | 7489826 | 1591 | LFDTV--FD--VDNDKERARDRGLE---P-----N-GFS-FDGA-DS------------- | 1622 |
| 1036 | 9502145 | 1528 | LYDTV--FD--VDNDKDRARSLGLE---P-----N-GFS-FDGA-DS------------- | 1559 |
| 1037 | 9502143 | 1545 | LHDTV--FD--VDNDKDRARSLGLE---P-----N-GFS-FDGA-DS------------- | 1576 |
| 1038 | 17646328 | 1133 | LYDTV--FD--VDDDKDRAREQGLE---P-----N-GFS-FEGA-DS------------- | 1164 |
| 1039 | 4582789 | 1063 | LYDTV--FD--VDNDKDRAQVQGLE---P-----N-GFS-FDGA-DA------------- | 1094 |
| 1040 | 7489274 | 1146 | LYDTV--FD--VDHDKERAQQCGLE---P-----N-GFS-FDGA-DA------------- | 1177 |
| 1041 | 2833389 | 1146 | LYDTV--FD--VDHDKERAQQCGLE---P-----N-GFS-FDGA-DA------------- | 1177 |
| 1042 | 15221083 | 943 | LFDTV--FD--VDHDKERAQAQVLE---P-----N-GFS-FDGA-DA------------- | 974 |
| 1043 | 7489827 | 153 | LFDTV--FD--VDNDKERARDRGLE---P-----N-GFS-FDGA-DS------------- | 184 |
| 1044 | 4582783 | 788 | LNDSV--FD--VDDDTIPSQFR------------N-GFT-FLNA-DE------------- | 815 |
| 1045 | 15717885 | 830 | LNDSV--FD--FDDETIPMEVR------------N-GFT-FVKA-DE------------- | 857 |
| 1046 | 15236819 | 956 | LNDSV--FD--IDDDTIPTQFQ------------N-GFT-FQTA-DEQLKIGMEIYLVWF | 996 |
| 1047 | 18461221 | 745 | LCDSV--FD--FDDETIPVELR------------N-GFT-FART-DE------------- | 772 |
| 1048 | 8901183 | 572 | LADTV--FD--VDGP---AGGPAQP---R-----N-GFV-FDGS-DD------------- | 600 |
| 1049 | 17227527 | 411 | LVNTV--FD--RDYDQNLPPEK------R-----N-GYV-FYQS-DN------------- | 439 |
| 1050 | 16329217 | 412 | LVNTV--FD--RDYDQNHPPEK------R-----N-GFV-FYQP-DE------------- | 440 |
| 1051 | 2811062 | 402 | LNDTV-----------QSYNEITKE---G-----N-GFS-FTNF-NA------------- | 426 |
| 1052 | 7484399 | 362 | LADTV--FD--VDGP---AGGPAQP---R-----N-GFV-FDGS-DD------------- | 390 |
| 1053 | 18309046 | 408 | LKDTI--HS--YN------KYTGI----G-----N-GFS-FTNY-NH------------- | 432 |
| 1054 | 15903076 | 402 | LRDTV----------------RAFN---PIEGSGT-GFS-FDNL-SP------------- | 426 |
| 1055 | 17229371 | 382 | LVDTV--SH--HDPINEAG---------------T-GYC-FDRY-EP------------- | 406 |
| 1056 | 15900991 | 402 | LRDTV----------------RAFN---PIEGSGT-GFS-FDNL-SP------------- | 426 |
| 1057 | 16124067 | 401 | LADTV------VDCALENLADG--S---A-----S-GFV-FNEC-EA------------- | 428 |
| 1058 | 16080147 | 403 | LYDTV--RA--YQEEEGTG---------------N-GFT-FSAF-NA------------- | 427 |
| 1059 | 16273270 | 401 | LADTV--VD--STSESIKAR----T---A-----T-GFV-FESA-TP------------- | 428 |
| 1060 | 15602409 | 401 | LADTV--VNATVENIKSRL---------A-----T-GFV-FEQA-NR------------- | 428 |
| 1061 | 15672681 | 410 | LKDTV--IP--FNPISK-------E---G-----T-GFG-FVDF-EG------------- | 434 |
| 1062 | 17938870 | 418 | LAETI--ID---------ANDAAIEAGVA-----T-GFQ-FEPA-NE------------- | 445 |
| 1063 | 16119514 | 397 | LAETI--ID---------ANDAAIEAGVA-----T-GFQ-FEPA-NE------------- | 424 |
| 1064 | 15613648 | 396 | LYDTI--QS--YNEEIG-------E---G-----N-GFS-FTHY-NA------------- | 420 |
| 1065 | 16331219 | 382 | LVDTV--SF--YDPINEAG---------------T-GYC-FDRY-EP------------- | 406 |
| 1066 | 17366711 | 405 | LKDTV--IP--FNPISK-------E---G-----T-GFG-FVDF-EG------------- | 429 |
| 1067 | 15805621 | 407 | LADTV-------------------P---P-----EVGFR-FADA-TA------------- | 424 |
| 1068 | 15620537 | 382 | LVDTV--FH--HD-------PRHAE---G-----N-GYC-FDRY-EP------------- | 406 |
| 1069 | 15641730 | 408 | LKDTV--ND--YDKFPERA---------------T-GFG-YQEP-TP------------- | 432 |
| 1070 | 7489711 | 621 | LRDTVAPFD--PFNDT------GL------------GWT-FDRA-EA------------- | 645 |
| 1071 | 15605532 | 407 | LADTV--AN-------------GI----------N-GFSFFNPH-DF------------- | 426 |
| 1072 | 15803938 | 401 | LADTV--SD--C--SLENLAD-GV----A-----S-GFV-FEDS-NA------------- | 428 |
| 1073 | 17367076 | 379 | LADTV-------------------P---P-----EVGFR-FADA-TA------------- | 396 |
| 1074 | 16766821 | 401 | LADTV------SDSSLENLAD-GI----A-----S-GFV-FEDS-NA------------- | 428 |
| 1075 | 16762766 | 401 | LADTV------SDSSLENLAD-GI----A-----S-GFV-FEDS-NA------------- | 428 |
| 1076 | 15824801 | 407 | LADTV--TD-------------GV----------N-GFS-FSNPHDF------------- | 426 |
| 1077 | 15966599 | 401 | LTDTI--ID--ANEAALSAKC-------A-----T-GFH-FLPV-TT------------- | 428 |
| 1078 | 15618857 | 406 | LADTV--IP-------------GV----------N-GFTFFDTN-NF------------- | 425 |
| 1079 | 17366350 | 375 | LKDTV--ED-------GRA-----------------GVL-FETY-HP------------- | 393 |
| 1080 | 15384987 | 560 | LRDTV--AP--FDP----FADTGL------------GWT-FDRA-EA------------- | 584 |
| 1081 | 2129898 | 675 | LRDTV--QP--FNPFDESGV----------------GWT-FDRA-EA------------- | 699 |
| 1082 | 2833384 | 675 | LRDTV--QP--FNPFDESGV----------------GWT-FDRA-EA------------- | 699 |
| 1083 | 15028467 | 617 | LRDTV--AP--FDP----FADTGL------------GWT-FDRA-EA------------- | 641 |
| 1084 | 15643657 | 407 | LADTV--KE--YD-------PQSME---G-----T-GFG-FKKY-DS------------- | 431 |

TABLE XXXV
Maize soluble starch synthase III (Du I)
“CTEND” Domain Alignments with other similar proteins

| SEQ | Accession | a.a | | a.a. |
|---|---|---|---|---|
| Id. No. | Number | # | (start) Sequence end | # |
| 1085 | MaizeDu1 | 423 | ---------------NGVDYALNRAISAW--F-DARSWFHSLCKRVMEQDWSWNRPALDY | 464 |
| 1086 | 7489826 | 1623 | ---------------NGVDYALNRAISAW--F-DARSWFHSLCKRVMEQDWSWNRPALDY | 1664 |
| 1087 | 9502145 | 1560 | ---------------NGVDYALNRAIGAW--F-DARSWFHSLCKRVMEQDWSWNRPALDY | 1601 |
| 1088 | 9502143 | 1577 | ---------------NGVDYALNRAIGAW--F-DARSWFHSLCKRVMEQDWSWNRPALDY | 1618 |
| 1089 | 17646328 | 1065 | ---------------NGVDYALDRAITTW--Y-DARSWFHSLCKRVMEQDWTWNRPALDY | 1206 |
| 1090 | 4582789 | 1095 | ---------------GGVDYALNRAISAW--Y-DGREWFNTLCKTVMEQDWSWNRPALDY | 1136 |
| 1091 | 7489274 | 1178 | ---------------GGVDYALNRALSAW--Y-DGRDWFNSLCKQVMEQDWSWNRPALDY | 1219 |
| 1092 | 2833389 | 1178 | ---------------GGVDYALNRALSAW--Y-DGRDWFNSLCKQVMEQDWSWNRPALDY | 1219 |
| 1093 | 15221083 | 975 | ---------------PGVDYALNRAISAW--Y-DGREWFNSLCKTVMEQDWSWNRPALEY | 1016 |
| 1094 | 7489827 | 185 | ---------------NGVDYALNRAISAW--F-DARSWFHSLCKRVMEQDWSWNRPALDY | 226 |
| 1095 | 4582783 | 816 | ---------------KGINDALVRAINLF--TNDPKSW-KQLVQKDMNIDFSWDSSAAQY | 857 |
| 1096 | 15717885 | 858 | ---------------QGLSSAMERAFNCY--T-RKPEVWKQLVQKDMTIDFSWDTSASQY | 899 |
| 1097 | 15236819 | 997 | SFTCPSLAEKGNVKKQGFNYALERAFNHY--K-KDEEKWMRLVEKVMSIDFSWGSSATQY | 1053 |
| 1098 | 18461221 | 773 | ---------------QDLSSCLERAFSYY--S-RKPMVWKQLVQKDMQIDFSWDSPASQY | 814 |
| 1099 | 8901183 | 601 | ---------------GALHGALDRALTLY--T-TQPERWAALQQDNMRLDVSWGKSAKSY | 642 |
| 1100 | 17227527 | 440 | ---------------QALESAMNRAIDLW--Y-QSPEQFQQLAIQGMKYDYSWNNPGTEY | 481 |
| 1101 | 16329217 | 441 | ---------------YALETALSRAIALY--K-DDPVAFKTLALQGMAYDYSWNKPGLQY | 482 |
| 1102 | 2811062 | 427 | ---------------HDMLYTIRRALSFY--R-QPSVW-EQLTERAMRGDYSWRRSANQY | 467 |
| 1103 | 7484399 | 391 | ---------------GALHGALDRALTLY--T-TQPERWAALQQDNMRLDVSWGKSAKSY | 432 |
| 1104 | 18309046 | 433 | ---------------NDLMHVIELALETY--D-DKEIW-RSLIIQAMDSDNSWNKSAEKY | 473 |
| 1105 | 15903076 | 427 | ---------------YWLNWTFQTALDLY--R-NHPDIWRNLQKQAMESDFSWDTACKSY | 468 |
| 1106 | 17229371 | 407 | ---------------LDLFTCM---IRAWEGF-RYKPQWQELQKRGMSQDFSWYKSAKEY | 447 |
| 1107 | 15900991 | 427 | ---------------YWLNWTFQTALDLY--R-NHPDIWRNLQKQAMESDFSWDTACKSY | 468 |
| 1108 | 16124067 | 429 | ---------------QALVKAIRRAFVLW--S-RPKHWRH-VQRHAMRLDFGWQLAAVDY | 469 |
| 1109 | 16080147 | 428 | ---------------HDLKFTIERALSFY--C-QQDVW-KSIVKTAMNADYSWGKSAKEY | 468 |
| 1110 | 16273270 | 429 | ---------------EALRHCLQRAFALW--Q-KPRAW-AMVRTDAMEQDFSWRKAAEQY | 469 |
| 1111 | 15602409 | 429 | ---------------EALRQALVNAFALW--Q-KQRLWF-TVRSVAMEQDFSWQISATGY | 469 |
| 1112 | 15672681 | 435 | ---------------QILVETINRALEVY--G-KEPEVLNKMVLSAMSKDFSWGTKAQQY | 476 |
| 1113 | 17938870 | 446 | ---------------DDLRAALERAISAY--N-D-RELFRRLQTQAMQANFSWDKSAAQY | 486 |
| 1114 | 16119514 | 425 | ---------------DDLRAALERAISAY--N-D-RELFRRLQTQAMQANFSWDKSAAQY | 465 |
| 1115 | 15613648 | 421 | ---------------HDFLYTIKRALRFY--R-TEKEW-ENLLLNIYSSEVGWDVSAKQY | 461 |
| 1116 | 16331219 | 407 | ---------------LDCFTAMVRAWEGF--R-FKADW-QKLQQRAMRADFSWYRSAGEY | 447 |
| 1117 | 17366711 | 430 | ---------------QILVETINRALEVY--G-KEPEVLNKMVLSAMSKDFSWGTKAQQY | 471 |
| 1118 | 15805621 | 425 | ---------------PAFLQACREAQAAF--Q-DPAQW-QTRATRAMSLDFSWDGPARQY | 465 |
| 1119 | 15620537 | 407 | ---------------LDLYTCLVRAWESY--Q-YQPQW-QKLQQRGMAVDLSWKQSGIAY | 447 |
| 1120 | 15641730 | 433 | ---------------EALLITMQRALLFY--L-QQPEEMLKVQQRAMQQNFSWEESAQEY | 474 |
| 1121 | 7489711 | 646 | ---------------NRMIDALSHCLTTY--R-NYKESWRACRARGMAEDLSWDHAAVLY | 687 |
| 1122 | 15605532 | 427 | ---------------YEFRNMLSEAVTTY--R-TNHDKWQHIVRACLDFSSDLETAANKY | 468 |
| 1123 | 15803938 | 429 | ---------------WSLLRAIRRAFVLW--S-RPSLW-RFVQRQAMAMDFSWQVAAKSY | 469 |
| 1124 | 17367076 | 397 | ---------------PAFLQACREAQAAF--Q-DPAQW-QTRATRAMSLDFSWDGPARQY | 437 |
| 1125 | 16766821 | 429 | ---------------WSLLRAIRRAFVLW--S-RPSLW-RFVQRQAMAMDFSWQVAAKSY | 469 |
| 1126 | 16762766 | 429 | ---------------WSLLRAIRRAFVLW--S-RPSLW-RFVQRQAMAMDFSWQVAAKSY | 469 |
| 1127 | 15834801 | 427 | ---------------HEFRNMLSKAIATY--R-DDQDKWQQIVRSCLEFSSDLETAANKY | 468 |
| 1128 | 15966599 | 429 | ---------------DGLRLAIRRVLRAY--N-EPKLW-ARLQYQGMKSDVSWAKSAERY | 469 |
| 1129 | 15618857 | 426 | ---------------NEFRAMLSNAVTTY--R-QEPDVWLNLIESGMLRASGLDAMAKHY | 467 |
| 1130 | 17366350 | 394 | ---------------EGLLYGVLRLF---------RLGAEEMGLRAMEKDFSWEGPARAY | 429 |
| 1131 | 15384987 | 585 | ---------------NRMIDALGHCLNTY--R-NYKESWRGLQARGMAQDLSWDHAA--- | 623 |
| 1132 | 2129898 | 700 | ---------------NKLMAALWNCLLTY--K-DYKKSWEGIQERGMSQDLSWDNAAQQY | 741 |
| 1133 | 2833384 | 700 | ---------------NKLMAALWNCLLTY--K-DYKKSWEGIQERGMSQDLSWDNAAQQY | 741 |
| 1134 | 15028467 | 642 | ---------------NRMIDALGHCLNTY--R-NYKESWRGLQARGMAQDLSWDHAA--- | 680 |
| 1135 | 15643657 | 432 | ---------------AHLLKAVSKALHFY--Y-REKDHWRRIMTNAMNTDLSWDRSAKEY | 473 |
| 1085 | MaizeDu1 | 465 | IELY-RSASKL | 474 |
| 1086 | 7489826 | 1665 | IELY-RSASKL | 1674 |
| 1087 | 9502145 | 1602 | IELY-HAARK | 1610 |
| 1088 | 9502143 | 1619 | IELY-HAARK | 1627 |
| 1089 | 17646328 | 1207 | MELY-HSARK | 1215 |
| 1090 | 4582789 | 1137 | LELY-HAACKL | 1146 |
| 1091 | 7489274 | 1220 | LELY-HAARKL | 1229 |
| 1092 | 2833389 | 1220 | LELY-HAARKL | 1229 |
| 1093 | 15221083 | 1017 | LELY-HSARK | 1025 |
| 1094 | 7489827 | 227 | IELY-RSASQI | 236 |
| 1095 | 4582783 | 858 | EELY | 861 |
| 1096 | 15717885 | 900 | EDIY-Q | 904 |
| 1097 | 15236819 | 1054 | EELYTRSVSR | 1063 |
| 1098 | 18461221 | 815 | ENLY-QSA | 821 |
| 1099 | 8901183 | 643 | VDVY-RSIS | 650 |
| 1100 | 17227527 | 482 | LNIY | 485 |
| 1101 | 16329217 | 483 | VEAY | 486 |
| 1102 | 2811062 | 468 | KQAY-EQLIK | 476 |
| 1103 | 7484399 | 433 | VDVY-RSIS | 440 |
| 1104 | 18309046 | 474 | KELY-EELIK | 482 |
| 1105 | 15903076 | 469 | LDLY-HS | 474 |
| 1106 | 17229371 | 448 | DKLY-RS | 453 |
| 1107 | 15900991 | 469 | LDLY-HS | 474 |
| 1108 | 16124067 | 470 | LSLY-R | 474 |
| 1109 | 16080147 | 469 | QRIF-EQVTR | 477 |
| 1110 | 16273270 | 470 | RTLY | 473 |
| 1111 | 15602409 | 470 | HALY-Q | 474 |
| 1112 | 15672681 | 477 | IELY-Q | 481 |
| 1113 | 17938870 | 487 | MALF-ES 492 | |
| 1114 | 16119514 | 466 | MALF-ES | 471 |
| 1115 | 15613648 | 462 | TALY | 465 |
| 1116 | 16331219 | 448 | IKVY-K | 452 |
| 1117 | 17366711 | 472 | IELY-Q | 476 |
| 1118 | 15805621 | 466 | LELY-R | 470 |
| 1119 | 15620537 | 448 | KQLY-AEA | 454 |
| 1120 | 15641730 | 475 | MKMY-RLA | 481 |
| 1122 | 15605532 | 469 | LEIY-K | 473 |
| 1123 | 15803938 | 470 | RELY | 473 |
| 1124 | 17367076 | 438 | LELY-R | 442 |
| 1125 | 16766821 | 470 | RELY | 473 |
| 1126 | 16762766 | 470 | RELY | 473 |
| 1127 | 15834801 | 469 | LEIY-Q | 473 |
| 1128 | 15966599 | 470 | VSLY | 473 |
| 1129 | 15618857 | 468 | VNLY-QS 473 | |
| 1130 | 17366350 | 430 | REVY-REA | 436 |
| 1131 | 15384987 | 624 | -ELY | 626 |
| 1132 | 2129898 | 742 | EEVL-VAA | 748 |
| 1133 | 2833384 | 742 | EEVL-VAA | 748 |
| 1134 | 15028467 | 681 | -ELY | 683 |
| 1135 | 15643657 | 474 | VDLY-KKA | 480 |

TABLE XXXVI
Identities of the Accession Numbers used in Tables. XXXI-XXXIV.

| Accession | Brief Description of sequences | score | |
|---|---|---|---|
| Id. | producing significant alignments | (bits) | E-value |
| gi\|7489826\|pir\|\|T01265 | starch synthase DULL1 - maize >gi\|30 . . . | 969 | 0.0 |
| gi\|9502145\|gb\|AAF88000.1\| | (AF258609) starch synthase III [A . . . | 815 | 0.0 |
| gi\|9502143\|gb\|AAF87999.1\|AF258608_1 | (AF258608) starch synth . . . | 811 | 0.0 |
| gi\|17646328\|gb\|AAL40942.1\|AF432915_1 | (AF432915) putative st . . . | 808 | 0.0 |
| gi\|4582789\|emb\|CAB40374.1\| | (AJ225088) Starch synthase isofo . . . | 758 | 0.0 |
| gi\|7489274\|pir\|T07663 | soluble starch synthase (EC 2.4.1. - ) . . . | 752 | 0.0 |
| gi\|2833389\|sp\|Q43846\|UGS4_SOLTU | Soluble glycogen [starch] s . . . | 749 | 0.0 |
| gi\|15221083\|ref\|NP_172637.1\| | (NM_101044) putative glycogen . . . | 734 | 0.0 |
| gi\|7489827\|pir\|T01266 | starch synthase DULL1 - maize (fragm . . . | 495 | e−139 |
| gi\|4582783\|emb\|CAB40375.1\| | (AJ006752) starch synthase, isof . . . | 394 | e−108 |
| gi\|15717885\|gb\|AAK97773.1\| | (AY044844) starch synthase isofo . . . | 363 | 2e−99 |
| gi\|15236819\|ref\|NP_193558.1\| | (NM_117934) starch synthase-li . . . | 361 | 1e−98 |
| gi\|18461221\|dbj\|BAB84418.1\| | (AP003292) putative starch synt . . . | 337 | 2e−91 |
| gi\|8901183\|gb\|AAC17971.2\| | (AF026422) soluble starch synthas . . . | 303 | 4e−81 |
| gi\|17227527\|ref\|NP_484075.1\| | (NC_003272) glycogen (starch) . . . | 300 | 2e−80 |
| gi\|16329217\|ref\|NP_439945.1\| | (NC_000911) glycogen (starch) . . . | 265 | 9e−70 |
| gi\|2811062\|sp\|O08328\|GLGA_BACST | Glycogen synthase (Starch [. . . | 249 | 6e−65 |
| gi\|7484399\|pir\|\|T07926 | probable starch synthase (EC 2.4.1.- . . . | 232 | 5e−60 |
| gi\|18309046\|ref\|NP_560980.1\| | (NC_003366) glycogen synthase . . . | 226 | 5e−58 |
| gi\|15903076\|ref\|NP_358626.1\| | (NC_003098) Glycogen synthase . . . | 224 | 2e−57 |
| gi\|17229371\|ref\|NP_485919.1\| | (NC_003272) glycogen synthase . . . | 222 | 8e−57 |
| gi\|15900991\|ref\|NP_345595.1\| | (NC_003028) glycogen synthase . . . | 221 | 9e−57 |
| gi\|16124067\|ref\|NP_407380.1\| | (NC_003143) glycogen synthase . . . | 220 | 2e−56 |
| gi\|16080147\|ref\|NP_390973.1\| | (NC_000964) starch (bacterial . . . | 215 | 6e−55 |
| gi\|16273270\|ref\|NP_439511.1\| | (NC_000907) glycogen synthase . . . | 214 | 1e−54 |
| gi\|15602409\|ref\|NP_245481.1\| | (NC_002663) GlgA [Pasteurella . . . | 214 | 1e−54 |
| gi\|15672681\|ref\|NP_266855.1\| | (NC_002662) glycogen synthase . . . | 212 | 8e−54 |
| gi\|17938870\|ref\|NP_535658.1\| | (NC_003306) glycogen synthase . . . | 211 | 1e−53 |
| gi\|16119514\|ref\|NP_396220.1\| | (NC_003064) AGR_pAT_410p [Agro . . . | 211 | 1e−53 |
| gi\|15613648\|ref\|NP_241951.1\| | (NC_002570) starch (bacterial . . . | 209 | 4e−53 |
| gi\|16331219\|ref\|NP_441947.1\| | (NC_000911) glycogen synthase . . . | 209 | 4e−53 |
| gi\|17366711\|sp\|Q9CHM9\|GLGA_LACLA | Glycogen synthase (Starch . . . | 209 | 5e−53 |
| gi\|15805621\|ref\|NP_294317.1\| | (NC_001263) glycogen synthase . . . | 200 | 3e−50 |
| gi\|15620537\|gb\|AAL03921.1\|U30252_9 | (U30252) GlgA [Synechoco . . . | 199 | 4e−50 |
| gi\|15641730\|ref\|NP_231362.1\| | (NC_002505) glycogen synthase . . . | 199 | 5e−50 |
| gi\|7489711\|pir\|T01209 | ADPglucose - starch glucosyltransfera . . . | 197 | 2e−49 |
| gi\|15605532\|ref\|NP_220318.1\| | (NC_000117) Glycogen Synthase . . . | 197 | 2e−49 |
| gi\|15803938\|ref\|NP_289974.1\| | (NC_002655) glycogen synthase . . . | 197 | 2e−49 |
| gi\|17367076\|sp\|Q9RWS1\|GLGA_DEIRA | Glycogen synthase (Starch . . . | 197 | 3e−49 |
| gi\|16766821\|ref\|NP_462436.1\| | (NC_003197) glycogen synthase . . . | 196 | 6e−49 |
| gi\|16762766\|ref\|NP_458383.1\| | (NC_003198) glycogen synthase . . . | 194 | 2e−48 |
| gi\|15834801\|ref\|NP_296560.1\| | (NC_002620) glycogen synthase . . . | 191 | 2e−47 |
| gi\|15966599\|ref\|NP_386952.1\| | (NC_003047) PROBABLE GLYCOGEN . . . | 189 | 4e−47 |
| gi\|15618857\|ref\|NP_225143.1\| | (NC_000922) Glycogen Synthase . . . | 189 | 5e−47 |
| gi\|17366350\|sp\|P58395\|GLGA_THECA | Glycogen synthase (Starch . . . | 187 | 1e−46 |
| gi\|15384987\|emb\|CAC59826.1\| | (AJ308110) soluble starch synth . . . | 187 | 2e−46 |
| gi\|2129898\|pir\|\|S61505 | UDPglucose - starch glucosyltransfera . . . | 185 | 9e−46 |
| gi\|2833384\|sp\|Q43093\|UGS3_PEA | Glycogen [starch] synthase, c . . . | 185 | 1e−45 |
| gi\|15028467\|gb\|AAK81729.1\|AF395537_1 | (AF395537) soluble sta . . . | 185 | 1e−45 |
| gi\|15643657\|ref\|NP_228703.1\| | (NC_000853) glycogen synthase . . . | 184 | 2e−45 |
| gi\|15232051\|ref\|NP_186767.1\| | (NM_110984) putative glycogen . . . | 181 | 1e−44 |
| gi\|13476305\|ref\|NP_107875.1\| | (NC_002678) glycogen synthase . . . | 181 | 2e−44 |
| gi\|7489710\|pir\|\|T01208 | ADPglucose - starch glucosyltransfera . . . | 179 | 6e−44 |
| gi\|146139\|gb\|AAA23870.1\| | (J02616) glycogen synthase (EC 2.4 . . . | 179 | 6e−44 |
| gi\|14279432\|gb\|AAK58596.1\|AF268969_2 | (AF268969) glycogen sy . . . | 179 | 7e−44 |
| gi\|15890897\|ref\|NP_356569.1\| | (NC_003063) AGR_L_1562p [Agrob . . . | 176 | 4e−43 |
| gi\|17366749\|sp\|Q9EUT5\|GLGA_RHITR | Glycogen synthase (Starch . . . | 175 | 7e−43 |
| gi\|16265159\|ref\|NP_437951.1\| | (NC_003078) putative glycogen . . . | 175 | 1e−42 |
| gi\|15895507\|ref\|NP_348856.1\| | (NC_003030) Glycogen synthase, . . . | 175 | 1e−42 |
| gi\|14495348\|gb\|AAK64284.1\|AF383878_1 | (AF383878) soluble sta . . . | 173 | 4e−42 |
| gi\|15606118\|ref\|NP_213495.1\| | (NC_000918) glycogen synthase . . . | 173 | 4e−42 |
| gi\|7489695\|pir\|\|T06798 | probable starch synthase (EC 2.4.1.- . . . | 170 | 3e−41 |
| gi\|16265834\|gb\|AAL16661.1\|AF419099_1 | (AF419099) putative so . . . | 169 | 6e−41 |
| gi\|8708896\|gb\|AAC17970.2\| | (AF026421) soluble starch synthas . . . | 167 | 2e−40 |
| gi\|6467503\|gb\|AAF13168.1\|AF173900_1 | (AF173900) granule boun . . . | 166 | 3e−40 |
| gi\|7529653\|emb\|CAB86618.1\| | (AJ269502) starch synthase IIa-1 . . . | 162 | 5e−39 |
| gi\|3192881\|gb\|AAC19119.1\| | (AF068834) starch synthase [Ipomo . . . | 162 | 6e−39 |
| gi\|8953571\|emb\|CAB96626.1\| | (AJ269503) starch synthase IIa-2 . . . | 161 | 2e−38 |
| gi\|8953573\|emb\|CAB96627.1\| | (AJ269504) starch synthase IIa-3 . . . | 159 | 4e−38 |
| gi\|5825480\|gb\|AAD53263.1\|AF155217_1 | (AF155217) starch synth . . . | 159 | 6e−38 |
| gi\|15237934\|ref\|NP_197818.1\| | (NM_122336) soluble starch syn . . . | 159 | 8e−38 |
| gi\|2833390\|sp\|Q43847\|UGS3_SOLTU | Glycogen [starch] synthase, . . . | 158 | 1e−37 |
| gi\|6690399\|gb\|AAF24126.1\|AF121673_1 | (AF121673) soluble star . . . | 157 | 3e−37 |
| gi\|1549232\|dbj\|BAA07396.1\| | (D38221) SSS1 [Oryza sativa] >gi . . . | 156 | 4e−37 |
| gi\|5295947\|dbj\|BAA81848.1\| | (AB026295) ESTs AU075322(C11109) . . . | 156 | 4e−37 |
| gi\|2833377\|sp\|Q40739\|UGS2_ORYSA | Soluble glycogen [starch] s . . . | 156 | 4e−37 |
| gi\|5834407\|gb\|AAD53959.1\|AF181035_3 | (AF181035) glycogen syn . . . | 154 | 2e−36 |
| gi\|17367070\|sp\|QPRNH6\|GLGA_RHOSH | Glycogen synthase (Starch . . . | 154 | 2e−36 |
| gi\|82478\|pir\|\|JQ0703 | UDPglucose - starch glucosyltransferase . . . | 154 | 2e−36 |
| gi\|15223331\|ref\|NP_174566.1\| | (NM_103023) starch synthase, p . . . | 152 | 8e−36 |
| gi\|2833382\|sp\|Q42968\|UGST_ORYGL | Granule-bound glycogen [sta . . . | 152 | 8e−36 |
| gi\|15983795\|gb\|AAL10494.1\| | (AY056803) At1g32900/F9L11_8 [Ar . . . | 150 | 2e−35 |
| gi\|7489712\|pir\|\|T01414 | ADPglucose - starch glucosyltransfera . . . | 150 | 2e−35 |
| gi\|297424\|emb\|CAA46294.1\| | (X65183) glycogen (starch) syntha . . . | 150 | 3e−35 |
| gi\|136758\|sp\|P19395\|UGST_ORYSA | Granule-bound glycogen [star . . . | 150 | 3e−35 |
| gi\|7798551\|gb\|AAC61675.2\| | (AF031162) granule-bound starch s . . . | 150 | 3e−35 |
| gi\|15451566\|gb\|AAK98690.1\|AC069158_2 | (AC069158) Putative st . . . | 148 | 1e−34 |
| gi\|12019656\|gb\|AAD45815.2\| | (AF168786) soluble starch syntha . . . | 148 | 1e−34 |
| gi\|5441242\|dbj\|BAA82346.1\| | (AB029546) granule-bound starch . . . | 147 | 3e−34 |
| gi\|6318538\|gb\|AAF06936.1\|AF110373_1 | (AF110373) granule-boun . . . | 146 | 5e−34 |
| gi\|6318540\|gb\|AAF06937.1\|AF110374_1 | (AF110374) granule-boun . . . | 145 | 7e−34 |
| gi\|6624287\|dbj\|BAA88512.1\| | (AB029064) starch synthase (GBSS . . . | 145 | 1e−33 |
| gi\|4760582\|dbj\|BAA77351.1\| | (AB019623) starch Synthase (GBSS . . . | 145 | 1e−33 |
| gi\|4760584\|dbj\|BAA77352.1\| | (AB019624) starch synthase (GBSS . . . | 144 | 2e−33 |
| gi\|15626365\|emb\|CAC69955.1\| | (AJ345045) granule-bound starch . . . | 144 | 2e−33 |
| gi\|15597361\|ref\|NP_250855.1\| | (NC_002516) probable glycogen . . . | 144 | 2e−33 |
| gi\|4588609\|gb\|AAD26156.1\|AF113844_1 | (AF113844) granule-boun . . . | 144 | 2e−33 |
| gi\|7484400\|pir\|\|T07924 | probable starch Synthase (EC 2.4.1.—. . . | 144 | 3e−33 |
| gi\|6624285\|dbj\|BAA88511.1\| | (AB029063) starch synthase (GBSS . . . | 144 | 3e−33 |
| gi\|6624281\|dbj\|BAA88509.1\| | (AB029061) starch synthase (GBSS . . . | 144 | 3e−33 |
| gi\|17736918\|gb\|AAL41028.1\| | (AF250137) mutant granule bound . . . | 144 | 3e−33 |
| gi\|11037536\|gb\|AAG27624.1\|AF286320_1 | (AF286320) granule bou . . . | 142 | 6e−33 |
| gi\|136755\|sp\|P09842\|UGST_HORVU | Granule-bound glycogen [star . . . | 140 | 2e−32 |
| gi\|18652407\|gb\|AAL77109.1\|AF474373_6 | (AF474373) granule-bou . . . | 140 | 2e−32 |
| gi\|4760580\|dbj\|BAA77350.1\| | (AB019622) starch synthase (GBSS . . . | 140 | 3e−32 |
| gi\|136765\|sp\|P27736\|UGST_WHEAT | Granule-bound glycogen [star . . . | 139 | 5e−32 |
| gi\|6624283\|dbj\|BAA88510.1\| | (AB029062) starch synthase (GBSS . . . | 139 | 7e−32 |
| gi\|228210\|prf\|\|1718316A | granule-bound starch synthase [Sola . . . | 138 | 1e−31 |
| gi\|4588607\|gb\|AAD26155.1\|AF113843_1 | (AF113843) granule-boun . . . | 138 | 1e−31 |
| gi\|16716335\|gb\|AAC17969.3\| | (AF026420) granule-bound starch . . . | 137 | 3e−31 |
| gi\|3493111\|gb\|AAD03013.1\| | (AF079293) granule-bound starch s . . . | 135 | 1e−30 |
| gi\|3493079\|gb\|AAD02997.1\| | (AF079277) granule-bound starch s . . . | 135 | 1e−30 |
| gi\|602594\|emb\|CAA58220.1\| | (X83220) starch (bacterial glycog . . . | 135 | 1e−30 |
| gi\|12003285\|gb\|AAG43519.1\|AF210699_1 | (AF210699) granule-bou . . . | 135 | 1e−30 |
| gi\|267196\|sp\|Q00775\|UGST_SOLTU | Granule-bound glycogen [star . . . | 134 | 2e−30 |
| gi\|3493113\|gb\|AAD03014.1\| | (AF079294) granule-bound starch s . . . | 134 | 2e−30 |
| gi\|3493115\|gb\|AAD03015.1\| | (AF079295) granule-bound starch s . . . | 132 | 7e−30 |
| gi\|136757\|sp\|P04713\|UGST_MAIZE | Granule-bound glycogen [star . . . | 132 | 9e−30 |
| gi\|3493119\|gb\|AAD03017.1\| | (AF079297) granule-bound starch s . . . | 131 | 1e−29 |
| gi\|3493055\|gb\|AAD02985.1\| | (AF079265) granule-bound starch s . . . | 131 | 1e−29 |
| gi\|12278481\|gb\|AAG48981.1\| | (AY010994) granule-bound starch . . . | 131 | 1e−29 |
| gi\|3493107\|gb\|AAD03011.1\| | (AF079291) granule-bound starch s . . . | 131 | 1e−29 |
| gi\|12278501\|gb\|AAG48991.1\| | (AY011004) granule-bound starch . . . | 131 | 2e−29 |
| gi\|2833383\|sp\|Q43092\|UGST_PEA | Granule-bound glycogen [starc . . . | 130 | 2e−29 |
| gi\|12278491\|gb\|AAG48986.1\| | (AY010999) granule-bound starch . . . | 130 | 2e−29 |
| gi\|3493121\|gb\|AAD03018.1\| | (AF079298) granule-bound starch s . . . | 130 | 4e−29 |
| gi\|12278477\|gb\|AAG48979.1\| | (AY010992) granule-bound starch . . . | 129 | 5e−29 |
| gi\|12278479\|gb\|AAG48980.1\| | (AY010993) granule-bound starch . . . | 129 | 6e−29 |
| gi\|12278485\|gb\|AAG48983.1\| | (AY010996) granule-bound starch . . . | 129 | 6e−29 |
| gi\|3493057\|gb\|AAD02986.1\| | (AF079266) granule-bound starch s . . . | 129 | 8e−29 |
| gi\|12278489\|gb\|AAG48985.1\| | (AY010998) granule-bound starch . . . | 129 | 9e−29 |
| gi\|12278495\|gb\|AAG48988.1\| | (AY011001) granule-bound starch . . . | 129 | 9e−29 |
| gi\|3493081\|gb\|AAD02998.1\| | (AF079278) granule-bound starch s . . . | 129 | 9e−29 |
| gi\|3493075\|gb\|AAD02995.1\| | (AF079275) granule-bound starch s . . . | 129 | 1e−28 |
| gi\|3493005\|gb\|AAD02960.1\| | (AF079240) granule-bound starch s . . . | 128 | 1e−28 |
| gi\|3493085\|gb\|AAD03000.1\| | (AF079280) granule-bound starch s . . . | 128 | 1e−28 |
| gi\|12278417\|gb\|AAG48949.1\| | (AY010962) granule-bound starch . . . | 128 | 2e−28 |
| gi\|3493027\|gb\|AAD02971.1\| | (AF079251) granule-bound starch s . . . | 128 | 2e−28 |
| gi\|2833381\|sp\|Q42857\|UGST_IPOBA | Granule-bound glycogen [sta . . . | 127 | 2e−28 |
| gi\|3493077\|gb\|AAD02996.1\| | (AF079276) granule-bound starch s . . . | 127 | 2e−28 |
| gi\|12278473\|gb\|AAG48977.1\| | (AY010990) granule-bound starch . . . | 127 | 2e−28 |
| gi\|12278487\|gb\|AAG48984.1\| | (AY010997) granule-bound starch . . . | 127 | 2e−28 |
| gi\|12278509\|gb\|AAG48995.1\| | (AY011008) granule-bound starch . . . | 127 | 2e−28 |
| gi\|3493089\|gb\|AAD03002.1\| | (AF079282) granule-bound starch s . . . | 127 | 2e−28 |
| gi\|12278514\|gb\|AAG48997.1\| | (AY011011) granule-bound starch . . . | 127 | 2e−28 |
| gi\|3493087\|gb\|AAD03001.1\| | (AF079281) granule-bound starch s . . . | 127 | 3e−28 |
| gi\|3493065\|gb\|AAD02990.1\| | (AF079270) granule-bound starch s . . . | 127 | 3e−28 |
| gi\|3493093\|gb\|AAD03004.1\| | (AF079284) granule-bound starch s . . . | 127 | 3e−28 |
| gi\|12278415\|gb\|AAG48948.1\| | (AY010961) granule-bound starch . . . | 127 | 3e−28 |
| gi\|3493095\|gb\|AAD03005.1\| | (AF079285) granule-bound starch s . . . | 127 | 3e−28 |
| gi\|12278463\|gb\|AAG48972.1\| | (AY010985) granule-bound starch . . . | 127 | 3e−28 |
| gi\|3493051\|gb\|AAD02983.1\| | (AF079263) granule-bound starch s . . . | 127 | 3e−28 |
| gi\|3493013\|gb\|AAD02964.1\| | (AF079244) granule-bound starch s . . . | 127 | 3e−28 |
| gi\|3493099\|gb\|AAD03007.1\| | (AF079287) granule-bound starch s . . . | 126 | 4e−28 |
| gi\|3493049\|gb\|AAD02982.1\| | (AF079262) granule-bound starch s . . . | 126 | 4e−28 |
| gi\|3493097\|gb\|AAD03006.1\| | (AF079286) granule-bound starch s . . . | 126 | 4e−28 |
| gi\|3493031\|gb\|AAD02973.1\| | (AF079253) granule-bound starch s . . . | 126 | 4e−28 |
| gi\|3493007\|gb\|AAD02961.1\| | (AF079241) granule-bound starch s . . . | 126 | 4e−28 |
| gi\|12278421\|gb\|AAG48951.1\| | (AY010964) granule-bound starch . . . | 126 | 4e−28 |
| gi\|3493091\|gb\|AAD03003.1\| | (AF079283) granule-bound starch s . . . | 126 | 4e−28 |
| gi\|3493059\|gb\|AAD02987.1\| | (AF079267) granule-bound starch s . . . | 126 | 4e−28 |
| gi\|12278453\|gb\|AAG48967.1\| | (AY010980) granule-bound starch . . . | 126 | 4e−28 |
| gi\|12278451\|gb\|AAG48966.1\| | (AY010979) granule-bound starch . . . | 126 | 4e−28 |
| gi\|13377473\|gb\|AAK20725.1\| | (AF318769) granule-bound starch . . . | 126 | 4e−28 |
| gi\|3493019\|gb\|AAD02967.1\| | (AF079247) granule-bound starch s . . . | 126 | 5e−28 |
| gi\|12278443\|gb\|AAG48962.1\| | (AY010975) granule-bound starch . . . | 126 | 6e−28 |
| gi\|12278471\|gb\|AAG48976.1\| | (AY010989) granule-bound starch . . . | 125 | 7e−28 |
| gi\|3493067\|gb\|AAD02991.1\| | (AF079271) granule-bound starch s . . . | 125 | 7e−28 |
| gi\|13774484\|gb\|AAK38881.1\| | (AF353519) granule-bound starch . . . | 125 | 7e−28 |
| gi\|12278493\|gb\|AAG48987.1\| | (AY011000) granule-bound starch . . . | 125 | 8e−28 |
| gi\|3493103\|gb\|AAD03009.1\| | (AF079289) granule-bound starch s . . . | 125 | 8e−28 |
| gi\|12278435\|gb\|AAG48958.1\| | (AY010971) granule-bound starch . . . | 125 | 9e−28 |
| gi\|12278423\|gb\|AAG48952.1\| | (AY010965) granule-bound starch . . . | 125 | 1e−27 |
| gi\|13194734\|gb\|AAK15529.1\| | (AF331953) granule-bound starch . . . | 125 | 1e−27 |
| gi\|3493071\|gb\|AAD02993.1\| | (AF079273) granule-bound starch s . . . | 125 | 1e−27 |
| gi\|12278419\|gb\|AAG48950.1\| | (AY010963) granule-bound starch . . . | 125 | 1e−27 |
| gi\|12278429\|gb\|AAG48955.1\| | (AY010968) granule-bound starch . . . | 124 | 2e−27 |
| gi\|17940620\|gb\|AAL49697.1\| | (AF445158) granule-bound starch . . . | 124 | 2e−27 |
| gi\|12278457\|gb\|AAG48969.1\| | (AY010982) granule-bound starch . . . | 124 | 2e−27 |
| gi\|3493061\|gb\|AAD02988.1\| | (AF079268) granule-bound starch s . . . | 124 | 2e−27 |
| gi\|3493069\|gb\|AAD02992.1\| | (AF079272) granule-bound starch s . . . | 124 | 2e−27 |
| gi\|12278449\|gb\|AAG48965.1\| | (AY010978) granule-bound starch . . . | 124 | 2e−27 |
| gi\|3493025\|gb\|AAD02970.1\| | (AF079250) granule-bound starch s . . . | 124 | 2e−27 |
| gi\|12278507\|gb\|AAG48994.1\| | (AY011007) granule-bound starch . . . | 124 | 2e−27 |
| gi\|12278511\|gb\|AAG48996.1\| | (AY011009) granule-bound starch . . . | 124 | 2e−27 |
| gi\|3493073\|gb\|AAD02994.1\| | (AF079274) granule-bound starch s . . . | 124 | 2e−27 |
| gi\|3493037\|gb\|AAD02976.1\| | (AF079256) granule-bound starch s . . . | 124 | 2e−27 |
| gi\|3493117\|gb\|AAD03016.1\| | (AF079296) granule-bound starch s . . . | 124 | 2e−27 |
| gi\|12278427\|gb\|AAG48954.1\| | (AY010967) granule-bound starch . . . | 124 | 2e−27 |
| gi\|12278425\|gb\|AAG48953.1\| | (AY010966) granule-bound starch . . . | 124 | 2e−27 |
| gi\|3493035\|gb\|AAD02975.1\| | (AF079255) granule-bound starch s . . . | 124 | 2e−27 |
| gi\|12278497\|gb\|AAG48989.1\| | (AY011002) granule-bound starch . . . | 124 | 2e−27 |
| gi\|3493021\|gb\|AAD02968.1\| | (AF079248) granule-bound starch s . . . | 124 | 2e−27 |
| gi\|3492999\|gb\|AAD02957.1\| | (AF079237) granule-bound starch s . . . | 124 | 3e−27 |
| gi\|3493041\|gb\|AAD02978.1\| | (AF079258) granule-bound starch s . . . | 124 | 3e−27 |
| gi\|13774482\|gb\|AAK38880.1\| | (AF353518) granule-bound starch . . . | 124 | 3e−27 |
| gi\|3493023\|gb\|AAD02969.1\| | (AF079249) granule-bound starch s . . . | 124 | 3e−27 |
| gi\|12278437\|gb\|AAG48959.1\| | (AY010972) granule-bound starch . . . | 123 | 3e−27 |
| gi\|3493083\|gb\|AAD02999.1\| | (AF079279) granule-bound starch s . . . | 123 | 3e−27 |
| gi\|3493101\|gb\|AAD03008.1\| | (AF079288) granule-bound starch s . . . | 123 | 4e−27 |
| gi\|12278475\|gb\|AAG48978.1\| | (AY010991) granule-bound starch . . . | 123 | 4e−27 |
| gi\|12278469\|gb\|AAG48975.1\| | (AY010988) granule-bound starch . . . | 123 | 4e−27 |
| gi\|13774486\|gb\|AAK38882.1\| | (AF353520) granule-bound starch . . . | 123 | 4e−27 |
| gi\|3493015\|gb\|AAD02965.1\| | (AF079245) granule-bound starch s . . . | 123 | 4e−27 |
| gi\|12278411\|gb\|AAG48946.1\| | (AY010959) granule-bound starch . . . | 123 | 5e−27 |
| gi\|12278503\|gb\|AAG48992.1\| | (AY011005) granule-bound starch . . . | 123 | 5e−27 |
| gi\|12278505\|gb\|AAG48993.1\| | (AY011006) granule-bound starch . . . | 123 | 5e−27 |
| gi\|12278413\|gb\|AAG48947.1\| | (AY010960) granule-bound starch . . . | 123 | 5e−27 |
| gi\|13377475\|gb\|AAK20726.1\| | (AF318770) granule-bound starch . . . | 123 | 5e−27 |
| gi\|3493011\|gb\|AAD02963.1\| | (AF079243) granule-bound starch s . . . | 122 | 6e−27 |
| gi\|3493001\|gb\|AAD02958.1\| | (AF079238) granule-bound starch s . . . | 122 | 6e−27 |
| gi\|12278431\|gb\|AAG48956.1\| | (AY010969) granule-bound starch . . . | 122 | 7e−27 |
| gi\|3493053\|gb\|AAD02984.1\| | (AF079264) granule-bound starch s . . . | 122 | 7e−27 |
| gi\|13774480\|gb\|AAK38879.1\| | (AF353517) granule-bound starch . . . | 122 | 7e−27 |
| gi\|17940628\|gb\|AAL49701.1\| | (AF445162) granule-bound starch . . . | 122 | 8e−27 |
| gi\|3493047\|gb\|AAD02981.1\| | (AF079261) granule-bound starch s . . . | 122 | 9e−27 |
| gi\|15054954\|gb\|AAK82785.1\|AF292516_1 | (AF292516) granule-bou . . . | 122 | 9e−27 |
| gi\|3493003\|gb\|AAD02959.1\| | (AF079239) granule-bound starch s . . . | 122 | 9e−27 |
| gi\|12278433\|gb\|AAG48957.1\| | (AY010970) granule-bound starch . . . | 122 | 9e−27 |
| gi\|3493009\|gb\|AAD02962.1\| | (AF079242) granule-bound starch s . . . | 122 | 1e−26 |
| gi\|15054956\|gb\|AAK82786.1\|AF292517_1 | (AF292517) granule-bou . . . | 122 | 1e−26 |
| gi\|3493043\|gb\|AAD02979.1\| | (AF079259) granule-bound starch s . . . | 122 | 1e−26 |
| gi\|15054922\|gb\|AAK82769.1\|AF292500_1 | (AF292500) granule-bou . . . | 122 | 1e−26 |
| gi\|15054984\|gb\|AAK82800.1\|AF292531_1 | (AF292531) granule-bou . . . | 122 | 1e−26 |
| gi\|17940655\|gb\|AAL49713.1\| | (AF445177) granule-bound starch . . . | 122 | 1e−26 |
| gi\|15054990\|gb\|AAK82803.1\|AF292534_1 | (AF292534) granule-bou . . . | 122 | 1e−26 |
| gi\|15054930\|gb\|AAK82773.1\|AF292504_1 | (AF292504) granule-bou . . . | 122 | 1e−26 |
| gi\|15054960\|gb\|AAK82788.1\|AF292519_1 | (AF292519) granule-bou . . . | 122 | 1e−26 |
| gi\|12278499\|gb\|AAG48990.1\| | (AY011003) granule-bound starch . . . | 121 | 1e−26 |
| gi\|15054968\|gb\|AAK82792.1\|AF292523_1 | (AF292523) granule-bou . . . | 121 | 1e−26 |
| gi\|3492995\|gb\|AAD02955.1\| | (AF079235) granule-bound starch s . . . | 121 | 1e−26 |
| gi\|17432494\|gb\|AAL38185.1\| | (AY062271) granule-bound starch . . . | 121 | 1e−26 |
| gi\|17940659\|gb\|AAL49715.1\| | (AF445179) granule-bound starch . . . | 121 | 2e−26 |
| gi\|3493017\|gb\|AAD02966.1\| | (AF079246) granule-bound starch s . . . | 121 | 2e−26 |
| gi\|17940626\|gb\|AAL49700.1\| | (AF445161) granule-bound starch . . . | 121 | 2e−26 |
| gi\|17940622\|gb\|AAL49698.1\| | (AF445159) granule-bound starch . . . | 121 | 2e−26 |
| gi\|3493033\|gb\|AAD02974.1\| | (AF079254) granule-bound starch s . . . | 120 | 2e−26 |
| gi\|17940636\|gb\|AAL49705.1\| | (AF445166) granule-bound starch . . . | 120 | 2e−26 |
| gi\|17940639\|gb\|AAL49706.1\| | (AF445168) granule-bound starch . . . | 120 | 2e−26 |
| gi\|15054970\|gb\|AAK82793.1\|AF292524_1 | (AF292524) granule-bou . . . | 120 | 3e−26 |
| gi\|3493063\|gb\|AAD02989.1\| | (AF079269) granule-bound starch s . . . | 120 | 3e−26 |
| gi\|3493045\|gb\|AAD02980.1\| | (AF079260) granule-bound starch s . . . | 120 | 4e−26 |
| gi\|17940634\|gb\|AAL49704.1\| | (AF445165) granule-bound starch . . . | 120 | 4e−26 |
| gi\|3493039\|gb\|AAD02977.1\| | (AF079257) granule-bound starch s . . . | 120 | 5e−26 |
| gi\|17940630\|gb\|AAL49702.1\| | (AF445163) granule-bound starch . . . | 119 | 5e−26 |
| gi\|17940632\|gb\|AAL49703.1\| | (AF445164) granule-bound starch . . . | 119 | 6e−26 |
| gi\|17940648\|gb\|AAL49710.1\| | (AF445173) granule-bound starch . . . | 119 | 7e−26 |
| gi\|17940624\|gb\|AAL49699.1\| | (AF445160) granule-bound starch . . . | 119 | 8e−26 |
| gi\|17940677\|gb\|AAL49723.1\| | (AF445189) granule-bound starch . . . | 118 | 2e−25 |
| gi\|17940644\|gb\|AAL49708.1\| | (AF445171) granule-bound starch . . . | 118 | 2e−25 |

| Glycosyl transferase family group1 (pfam 00534)domain (“GLYTR”) of maize | | |
|---|---|---|
| GBSS | | |
| NKEALQAEVGLPVDRNIPLVAFIGRLEEQ | | Seq ID. No. 1136 |
| KGPDVMAAAIPQLMEMVEDVQIVLLGTGKKKFERMLMSAEEKFPGKVRAVVKFNAALA | | |
| HHIMAGADVLAVTSRFEPCGLIQLQGMRYGTPCACASTGGLVDTIIEGKTGFHMGRLS | | |
| VDCNVVEPADVKKVATTLQRAIK | | |
| Glycosyl transferase family group1 (pfam 00534)domain (“GLYTR”) of maize | | |
| SSI | | |
| LPIRPDVPLIGFIGRLDYQKGIDLIQLIIPDLMREDVQFVMLGSGDPELEDWMRSTESIFKDKFRGWV | | Seq ID. No. 1137 |
| GFSVPVSHRITAGCDILLMPSRFEPCGLNQLYAMQYGTVPVVHATGGLRDTVENFNPF | | |
| GENGEQGTGWAFAPLTTENMFVDIANCNIYIQGTQVLLGRANEARHVKRLHVGPCR | | |
| Glycosyl transferase family group1 (pfam 00534)domain (GLYTR) of maize | | |
| SSIIa | | |
| KAALQRELGLEVRDDVPLLGFIGRLDGQKGVDIIGDAMPWIAGQDVQLVMLGTGRADLERMLQHLEREH | | Seq ID. No. 1138 |
| PNKVRGWVGFSVPMAHRITAGADVLVMPSRFEPCGLNQLYAMAYGTVPVVHAVGGLRDTVAPFDPFGD | | |
| AGLGWTFDRAEANKLIEALR | | |
| Glycosyl transferase family group1 (pfam 00534)domain (GLYTR) of maize | | |
| SSIIb | | |
| QVRDDVPLIGFIGRLDHQKGVDIIADAIHWIAGQDVQLVMLGTGRADLEDMLRRFESEHSDKVRAWVGFS | | Seq ID. No. 1139 |
| VPLAHRITAGADILLMPSRFEPCGLNQLYAMAYGTVPVVHAVGGLRDTVAPFDPFNDTGLGWTFDRAEA | | |
| NRMID | | |
| Glycosyl transferase family group1 (pfam 00534)domain (GLYTR) of maize | | |
| DU1 | | |
| PVV GIVTRLTAQK GIHLIKHAIHRTLERNGQVV LLGSAPDSRI QADFVNLANT LHGVNHGQVR | | Seq ID. No. 1140 |
| LSLTYDEPLS HLIYAGSDFILVPSIFEPCG LTQLVAMRYG TIPIVRKTGG LFDTVFDVDN | | |
| Nucleotide sequence of maize GBSS | | |
| 1 | CAGCGACCTA TTACACAGCC CGCTCGGGCC CGCGACGTCG GGACACATCT TCTTCCCCCT | Seq ID. No. 1141 |
| 61 | TTTGGTGAAG CTCTGCTCGC AGCTGTCCGG CTCCTTGGAC GTTCGTGTGG CAGATTCATC | |
| 121 | TGTTGTCTCG TCTCCTGTGC TTCCTGGGTA GCTTGTGTAG TGGAGCTGAC ATGGTCTGAG | |
| 181 | CAGGCTTAAA ATTTGCTCGT AGACGAGGAG TACCAGCACA GCACGTTGCG GATTTCTCTG | |
| 241 | CCTGTGAAGT GCAACGTCTA GGATTGTCAC ACGCCTTGGT CGCGTCGCGT CGCGTCGCGT | |
| 301 | CGATGCGGTG GTGAGCAGAG CAGCAACAGC TGGGCGGCCC AACGTTGGCT TCCGTGTCTT | |
| 361 | CGTCGTACGT ACGCGCGCGC CGGGGACACG CAGCAGAGAG CGGAGAGCGAGCCGTGCACG | |
| 421 | GGGAGGTGGT GTGGAAGTGG AGCCGCGCGC CCGGCCGCCC GCGCCCGGTGGGCAACCCAA | |
| 481 | AAGTACCCAC GACAAGCGAA GGCGCCAAAG CGATCCAAGC TCCGGAACGC AACAGCATGC | |
| 541 | GTCGCGTCGG AGAGCCAGCC ACAAGCAGCC GAGAACCGAA CCGGTGGGCG ACGCGTCATG | |
| 601 | GGACGGACGC GGGCGACGCT TCCAAACGGG CCACGTACGC CGGCGTGTGC GTGCGTGCAG | |
| 661 | ACGACAAGCC AAGGCGAGGC AGCCCCCGAT CGGGAAAGCG TTTTGGGCGC GAGCGCTGGC | |
| 721 | GTGCGGGTCA GTCGCTGGTG CGCAGTGCCG GGGGGAACGG GTATCGTGGG GGGCGCGGGC | |
| 781 | GGAGGAGAGC GTGGCGAGGG CCGAGAGCAG CGCGCGGCCG GGTCACGCAA CGCGCCCCAC | |
| 841 | GTACTGCCCT CCCCCTCCGC GCGCGCTAGA AATACCGAGG CCTGGACCGG GGGGGGGCCC | |
| 901 | CGTCACATCC ATCCATCGAC CGATCGATCG CCACAGCCAA CACCACCCGC CGAGGCGACG | |
| 961 | CGACAGCCGC CAGGAGGAAG GAATAAACTC ACTGCCAGCC AGTGAAGGGG GAGAAGTGTA | |
| 1021 | CTGCTCCGTC GACCAGTGCG CGCACCGCCC GGCAGGGCTG CTCATCTCGT CGACGACCAG | |
| 1081 | GTTCTGTTCC GTTCCGATCC GATCCGATCC TGTCCTTGAG TTTCGTCCAG ATCCTGGCGC | |
| 1141 | GTATCTGCGT GTTTGATGAT CCAGGTTCTT CGAACCTAAA TCTGTCCGTG CACACGTCTT | |
| 1201 | TTCTCTCTCT CCTACGCAGT GGATTAATCG GCATGGCGGC TCTGGCCACG TCGCAGCTCG | |
| 1261 | TCGCAACGCG CGCCGGCCTG GGCGTCCCGG ACGCGTCCAC GTTCCGCCGC GGCGCCGCGC | |
| 1321 | AGGGCCTGAG GGGGGCCCGG GCGTCGGCGG CGGCGGACAC GCTCAGCATG CGGACCAGCG | |
| 1381 | CGCGCGCGGC GCCCAGGCAC CAGCAGCAGG CGCGCCGCGG GGGCAGGTTC CCGTCGCTCG | |
| 1441 | TCGTGTGCGC CAGCGCCGGC ATGAACGTCG TCTTCGTCGG CGCCGAGATG GCGCCGTGGA | |
| 1501 | GCAAGACCGG CGGCCTCGGC GACGTCCTCG GCGGCCTGCC GCCGGCCATG GCCGTAAGCG | |
| 1561 | CGCGCACCGA GACATGCATC CGTTGGATCG CGTCTTCTTC GTGCTCTTGC CGCGTGCATG | |
| 1621 | ATGCATGTGT TTCCTCCTGG CTTGTGTTCG TGTATGTGAC GTGTTTGTTC GGGCATGCAT | |
| 1681 | GCAGGCGAAC GGGCACCGTG TCATGGTCGT CTCTCCCCGC TACGACCAGT ACAAGGACGC | |
| 1741 | CTGGGACACC AGCGTCGTGT CCGAGGTACG GCCACCGAGA CCAGATTCAG ATCACAGTCA | |
| 1801 | CACACACCGT CATATGAACC TTTCTCTGCT CTGATGCCTG CAACTGCAAA TGCATGCAGA | |
| 1861 | TCAAGATGGG AGACGGGTAC GAGACGGTCA GGTTCTTCCA CTGCTACAAG CGCGGAGTGG | |
| 1921 | ACCGCGTGTT CGTTGACCAC CCACTGTTCC TGGAGAGGGT GAGACGAGAT CTGATCACTC | |
| 1981 | GATACGCAAT TACCACCCCA TTGTAAGCAG TTACAGTGAG CTTTTTTTCC CCCCGGCCTG | |
| 2041 | GTCGCTGGTT TCAGGTTTGG GGAAAGACCG AGGAGAAGAT CTACGGGCCT GTCGCTGGAA | |
| 2101 | CGGACTACAG GGACAACCAG CTGCGGTTCA GCCTGCTATG CCAGGTCAGG ATGGCTTGGT | |
| 2161 | ACTACAACTT CATATCATCT GTATGCAGCA GTATACACTG ATGAGAAATG CATGCTGTTC | |
| 2221 | TGCAGGCAGC ACTTGAAGCT CCAAGGATCC TGAGCCTCAA CAACAACCCA TACTTCTCCG | |
| 2281 | GACCATACGG TAAGAGTTGC AGTCTTCGTA TATATATCTG TTGAGCTCGA GAATCTTCAC | |
| 2341 | AGGAAGCGGC CCATCAGACG GACTGTCATT TTACACTGAC TACTGCTGCT GCTCTTCGTC | |
| 2401 | CATCCATACA AGGGGAGGAC GTCGTGTTCG TCTGCAACGA CTGGCACACC GGCCCTCTCT | |
| 2461 | CGTGCTACCT CAAGAGCAAC TACCAGTCCC ACGGCATCTA CAGGGACGCA AAGGTTGCCT | |
| 2521 | TCTCTGAACT GAACAACGCC GTTTTCGTTC TCCATGCTCG TATATACCTC GTCTGGTAGT | |
| 2581 | GGTGGTGCTT CTCTGAGAAA CTAACTGAAA CTGACTGCAT GTCTGTCTGA CCATCTTCAC | |
| 2641 | GTACTACCAG ACCGCTTTCT GCATCCACAA CATCTCCTAC CAGGGCCGGT TCGCCTTCTC | |
| 2701 | CGACTACCCG GAGCTGAACC TCCCGGAGAG ATTCAAGTCG TCCTTCGATT TCATCGACGG | |
| 2761 | GTCTGTTTTC CTGCGTGCAT GTGAACATTC ATGAATGGTA ACCCACAACT GTTCGCGTCC | |
| 2821 | TGCTGGTTCA TTATCTGACC TGATTGCATT ATTGCAGCTA CGAGAAGCCC GTGGAAGGCC | |
| 2881 | GGAAGATCAA CTGGATGAAG GCCGGGATCC TCGAGGCCGA CAGGGTCCTC ACCGTCAGCC | |
| 2941 | CCTACTACGC CGAGGAGCTC ATCTCCGGCA TCGCCAGGGG CTGCGAGCTC GACAACATCA | |
| 3001 | TGCGCCTCAC CGGCATCACC GGCATCGTCA ACGGCATGGA CGTCAGCGAG TGGGACCCCA | |
| 3061 | GCAGGGACAA GTACATCGCC GTGAAGTACG ACGTGTCGAC GGTGAGCTGG CTAGCTCTGA | |
| 3121 | TTCTGCTGCC TGGTCCTCCT GCTCATCATG CTGGTTCGGT ACTGACGCGG CAAGTGTACG | |
| 3181 | TACGTGCGTG CGACGGTGGT GTCCGGTTCA GGCCGTGGAG GCCAAGGCGC TGAACAAGGA | |
| 3241 | GGCGCTGCAG GCGGAGGTCG GGCTCCCGGT GGACCGGAAC ATCCCGCTGG TGGCGTTCAT | |
| 3301 | CGGCAGGCTG GAAGAGCAGA AGGGCCCCGA CGTCATGGCG GCCGCCATCC CGCAGCTCAT | |
| 3361 | GGAGATGGTG GAGGACGTGC AGATCGTTCT GCTGGTACGT GTGCGCCGGC CGCCACCCGG | |
| 3421 | CTACTACATG CGTGTATCGT TCGTTCTACT GGAACATGCG TGTGAGCAAC GCGATGGATA | |
| 3481 | ATGCTGCAGG GCACGGGCAA GAAGAAGTTC GAGCGCATGC TCATGAGCGC CGAGGAGAAG | |
| 3541 | TTCCCAGGCA AGGTGCGCGC CGTGGTCAAG TTCAACGCGG CGCTGGCGCA CCACATCATG | |
| 3601 | GCCGGCGCCG ACGTGCTCGC CGTCACCAGC CGCTTCGAGC CCTGCGGCCT CATCCAGCTG | |
| 3661 | CAGGGGATGC GATACGGAAC GGTACGAGAG AAAAAAAAAA TCCTGAATCC TGACGAGAGG | |
| 3721 | GACAGAGACA GATTATGAAT GCTTCATCGA TTTGAATTGA TTGATCGATG TCTCCCGCTG | |
| 3781 | CGACTCTTGC AGCCCTGCGC CTGCGCGTCC ACCGGTGGAC TCGTCGACAC CATCATCGAA | |
| 3841 | GGCAAGACCG GGTTCCACAT GGGCCGCCTC AGCGTCGACG TAAGCCTAGC TCTGCCATGT | |
| 3901 | TCTTTCTTCT TTCTTTCTGT ATGTATGTAT GAATCAGCAC CGCCGTTCTT GTTTCGTCGT | |
| 3961 | CGTCCTCTCT TCCCAGTGTA ACGTCGTGGA GCCGGCGGAC GTCAAGAAGG TGGCCACCAC | |
| 4021 | ATTGCAGCGC GCCATCAAGG TGGTCGGCAC GCCGGCGTAC GAGGAGATGG TGAGGAACTG | |
| 4081 | CATGATCCAG GATCTCTCCT GGAAGGTACG TACGCCCGCC CCGCCCCGCC CCGCCAGAGC | |
| 4141 | AGAGCGCCAA GATCGACCGA TCGACCGACC ACACGTACGC GCCTCGCTCC TGTCGCTGAC | |
| 4201 | CGTGGTTTAA TTTGCGAAAT GCGCAGGGCC CTGCCAAGAA CTGGGAGAAC GTGCTGCTCA | |
| 4261 | GCCTCGGGGT CGCCGGCGGC GAGCCAGGGG TCGAAGGCGA GGAGATCGCG CCGCTCGCCA | |
| 4321 | AGGAGAACGT GGCCGCGCCC TGAAGAGTTC GGCCTGCAGG GCCCCTGATC TCGCGCGTGG | |
| 4381 | TGCAAAGATG TTGGGACATC TTCTTATATA TGCTGTTTCG TTTATGTGAT ATGGACAAGT | |
| 4441 | ATGTGTAGCT GCTTGCTTGT GCTAGTGTAA TGTAGTGTAG TGGTGGCCAG TGGCACAACC | |
| 4501 | TAATAAGCGC ATGAACTAAT TGCTTGCGTG TGTAGTTAAG TACCGATCGG TAATTTTATA | |
| 4561 | TTGCGAGTAA ATAAATGGAC CTGTAGTGGT GGAGTAAATA ATCCCTGCTG TTCGGTGTTC | |
| 4621 | TTATCGCTCC TCGTATAGAT ATTATATAGA GTACATTTTT CTCTCTCTGA ATCCTACGTT | |
| 4681 | TGTGAAATTT CTATATCATT ACTGTAAAAT TTCTGCGTTC CAAAAGAGAC CATAGCCTAT | |
| 4741 | CTTTGGCCCT GTTTGTTTCG GCTTCTGGCA GCTTCTGGCC ACCAAAAGCT GCTGCGGACT | |
| Nucleotide sequence of maize SSI | | |
| 1 | GAATTCGCGG CCGCCTTATT TCTGGTTGGC CACATACATC ATCCAAAAAA CTTTATTATT | Seq ID. No. 1142 |
| 61 | GAATTACAAC TAATAAGCAA TCTAAAAGAG GGCACCACCA ATGATGTGTT GTTGGTAGGA | |
| 121 | GGCCGCTGGG TCTGTCAAAG CAAGITGGAC AAAGGGCAAC AATTGTTGTA GTTGTAAGAG | |
| 181 | GGTTGCGGGG TTAGCCGCAA ACTGCTGGTA GAAAGGCAGC AACTGTTGCT GTGTCAAGAA | |
| 241 | GGAAGCACGG TTTGCTGCAG CTGTTGTGCC CTGATGGTTT GTACGAATGA CTGCACCAAA | |
| 301 | GATAGGGCTG GCGATTGTTG AAACAACAAG GGCGATAAAG GTATGTTGCT TGCTGCGATT | |
| 361 | GCTTGTTGAA GCCTATATGG TTGAAGAGCT GGGTTTTCAC ATATTGAAGC TATAATTGAT | |
| 421 | GGAAGGTATG GGGGAAGAAG GGAAGCTATA GGAGCTTGTG AGCATTGAGG GAAAATTGTC | |
| 481 | GCGTTAGCAA CACATGTAGA AAGAGCAAGG AGCATAAGGA GGGAAAATAT CTTGGTCGCC | |
| 541 | ATTGTTGCGC GCGATCCACG GCCCCCGCCC CCCGCGCTCC TGTCTGCTCT CCCTCTCCGC | |
| 601 | AATGGCGACG CCCTCGGCCG TGGGCGCCGC GTGCCTCCTC CTCGCGCGGG CCGCCTGGCC | |
| 661 | GGCCGCCGTC GGCGACCGGG CGCGCCCGCG GCGGCTCCAG CGCGTGCTGC GCCGCCGGTG | |
| 721 | CGTCGCGGAG CTGAGCAGGG AGGGGCCCGC GCCGCGCCCG CTGCCACCCG CGCTGCTGGC | |
| 781 | GCCCCCGCTC GTGCCCGGCT TCCTCGCGCC GCCGGCCGAG CCCACGGGTG AGCCGGCATC | |
| 841 | GACGCCGCCG CCCGTGCCCG ACGCCGGCCT GGGGGACCTC GGTCTCGAAG CTGAAGGGAT | |
| 901 | TGCTGAAGGT TCCATCGATA ACACAGTAGT TGTGGCAAGT GAGCAAGATT CTGAGATTGT | |
| 961 | GGTTGGAAAG GAGCAAGCTC GAGCTAAAGT AACACAAAGC ATTGTCTTTG TAACCGGCGA | |
| 1021 | AGCTTCTCCT TATGCAAAGT CTGGGGGTCT AGGAGATGTT TGTGGTTCAT TGCCAGTTGC | |
| 1081 | TCTTGCTGCT CGTGGTCACC GTGTGATGGT TGTAATGCCC AGATATTTAA ATGGTACCTC | |
| 1041 | CGATAAGAAT TATGCAAATG CATTTTACAC AGAAAAACAC ATTCGGATTC CATGCTTTGG | |
| 1201 | CGGTGAACAT GAAGTTACCT TCTTCCATGA GTATAGAGAT TCAGTTGACT GGGTGTTTGT | |
| 1261 | TGATCATCCC TCATATCACA GACCTGGAAA TTTATATGGA GATAAGTTTG GTGCTTTTGG | |
| 1321 | TGATAATCAG TTCAGATACA CACTCCTTTG CTATGCTGCA TGTGAGGCTC CTTTGATCCT | |
| 1381 | TGAATTGGGA GGATATATTT ATGGACAGAA TTGCATGTTT GTTGTCAATG ATTGGCATGC | |
| 1441 | CAGTCTAGTG CCAGTCCTTC TTGCTGCAAA ATATAGACCA TATGGTGTTT ATAAAGACTC | |
| 1501 | CCGCAGCATT CTTGTAATAC ATAATTTAGC ACATCAGGGT GTAGAGCCTG CAAGCACATA | |
| 1561 | TCCTGACCTT GGGTTGCCAC CTGAATGGTA TGGAGCTCTG GAGTGGGTAT TCCCTGAATG | |
| 1621 | GGCGAGGAGG CATGCCCTTG ACAAGGGTGA GGCAGTTAAT TTTTTGAAAG GTGCAGTTGT | |
| 1681 | GACAGCAGAT CGAATCGTGA CTGTCAGTAA GGGTTATTCG TGGGAGGTCA CAACTGCTGA | |
| 1741 | AGGTGGACAG GGCCTCAATG AGCTCTTAAG CTCCAGAAAG AGTGTATTAA ACGGAATTGT | |
| 1801 | AAATGGAATT GACATTAATG ATTGGAACCC TGCCACAGAC AAATGTATCC CCTGTCATTA | |
| 1861 | TTCTGTTGAT GACCTCTCTG GAAAGGCCAA ATGTAAAGGT GCATTGCAGA AGGAGCTGGG | |
| 1921 | TTTACCTATA AGGCCTGATG TTCCTCTGAT TGGCTTTATT GGAAGGTTGG ATTATCAGAA | |
| 1981 | AGGCATTGAT CTCATTCAAC TTATCATACC AGATCTCATG CGGGAAGATG TTCAATTTGT | |
| 2041 | CATGCTTGGA TCTGGTGACC CAGAGCTTGA AGATTGGATG AGATCTACAG AGTCGATCTT | |
| 2101 | CAAGGATAAA TTTCGTGGAT GGGTTGGATT TAGTGTTCCA GTTTCCCACC GAATAACTGC | |
| 2161 | CGGCTGCGAT ATATTGTTAA TGCCATCCAG ATTCGAACCT TGTGGTCTCA ATCAGCTATA | |
| 2221 | TGCTATGCAG TATGGCACAG TTCCTGTTGT CCATGCAACT GGGGGCCTTA GAGATACCGT | |
| 2281 | GGAGAACTTC AACCCTTTCG GTGAGAATGG AGAGCAGGGT ACAGGGTGGG CATTCGCACC | |
| 2341 | CCTAACCACA GAAAACATGT TTGTGGACAT TGCGAACTGC AATATCTACA TACAGGGAAC | |
| 2401 | ACAAGTCCTC CTGGGAAGGG CTAATGAAGC GAGGCATGTC AAAAGACTTC ACGTGGGACC | |
| 2461 | ATGCCGCTGA ACAATACGAA CAAATCTTCC AGTGGGCCTT CATCGGATCG ACCCGATGTT | |
| 2521 | CAATGGAAAA AAGGGACCAA AGTTGGTTGG TTCCTTGAAG ATTATCAGTT CATCATCCTA | |
| 2581 | TAGTAAGCTG AATGATGAAA GAAAACCCCT GTACATTACA TGGAAGGCAG ACCGGCTATT | |
| 2641 | GGCTCCATTG CTCCAATGTC TGCTTTGGCT GCCTTGCCTC GATGGACCGG ATGCAGTGAG | |
| 2701 | GAATCCAGNC GAACGACAGT TTTGAAGGAT AGGAAGGGGA GCTGGAAGCA GTCACGCAGG | |
| 2761 | CAGGCAAGCC TTCGCCGTTA ATTCATATGG AACAAGCTGG AGTCAGTTTC TGCTGTGCCA | |
| 2821 | CTCACTGTTT ACCTTAAGAT TATTACCTGT GTTGTTCTCC TTTGCTCGTT AGGGCTGATA | |
| 2881 | ACATAATGAC TCATTAAGAA TATAATTCAC TCTGCCTCGT TTTTAATCTT AAGTGAAGTC | |
| 2941 | GAGATCTACT TCGTCATTCC TTCCCCGTTT AAAAAAAAAA AAAAAAAAAA A | |
| Nucleotide sequence of maize SSIIa | | |
| 1 | GCGGCCGCTT TTTTTTAAAA TCTGTGGAGA AGGCTTTATT ATTAGCTCAT AATACTTTTG | Seq ID. No. 1143 |
| 61 | ATGCATCAAT AGGCCTCTCC CACCTCACAT TAGTCTCAAC ATTGACTTCA TATTATACAA | |
| 121 | CCCTAAGAAG GTTGATCGGC TGCAACAATT GAAATCTAAA GACTACGTCA AGCGCTTCCC | |
| 181 | TCTTGTGATA TCATCAGTTT GCTGGAACCT TGTAGCTTCC GCACTGGAAG CCATGCTCAA | |
| 241 | ATGGCCTCTT ACACTGCTCA GCAACATACA CAACCTTCTC CATGCACACA ACCTTCTCAG | |
| 301 | TTTCTTTGGT GACCTTGGAG CACAAGCAAG AAACGTTIGC CTTCTTGATG ACACCACAGC | |
| 361 | AAGCATCCGA AGGAGGGACC TTTGGATTAG CTGGGAACAT CACGTATGTC TTGCAGTCAC | |
| 421 | TTATCAGGCT TTGGAGGTCA TTCTCACAGT CTCCATCAGC ATGAGAGCCT ATGGCAAACA | |
| 481 | TAGCTGCTAC AAAGGAGAGG GCTAAGAACA GGCTCAACAG TTTTGCCATG TTTACTAGTT | |
| 541 | CTTGGGGGAC GCCTATTGAA AGCTGTCCGC TGCCCACCGC GTGCGCTGCG CTCCCGTGCG | |
| 601 | GCACTGCCGG CGGCGCGACC CCGCGCCCAT TGGACGAGCT TCCGCCCCGC CCCGATCGAT | |
| 661 | CCCGCCGCCA TGTCGTCGGC GGCCGTGTCG TCCTCTTCCT CCACCTTCTT CCTCGCGCTC | |
| 721 | GCCTCCGCCT CCCCCGGGGG CCGCAGGCGG GCTAGGGTCG GCTCCTCGCC GTTCCACACC | |
| 781 | GGCGCCAGCC TGAGTTTCGC GTTCTGGGCG CCACCGTCGC CGCCGCGCGC GCCCCGGGAC | |
| 841 | GCAGCGCTGG TGCGCGCTGA GGCTGAGGCC GGGGGCAAGG ACGCGCCGCC GGAGAGGAGC | |
| 901 | GGCGACGCCG CCAGGTTGCC CCGCGCTCGG CGCAATGCGG TCTCCAAACG GAGGGATCCT | |
| 961 | CTTCAGCCGG TCGGCCGGTA CGGCTCCGCG ACGGGAAACA CGGCCAGGAC CGGCGCCGCG | |
| 1021 | TCCTGCCAGA ACGCCGCATT GGCGGACGTT GAGATCAAGT CCATCGTCGC CGCGCCGCCG | |
| 1081 | ACGAGCATAG TGAAGTTCCC AGCGCCGGGC TACAGGATGA TCCTTCCCTC TGGGGACATA | |
| 1041 | GCGCCGGAGA CTGTCCTCCC AGCCCCGAAG CCACTGCATG AATCGCCTGC GGTTGACGGA | |
| 1201 | GATTCAAATG GAATTGCACC TCCTACAGTT GAGCCATTAG TACAGGAGGC CACTTGGGAT | |
| 1261 | TTCAAGAAAT ACATCGGTTT TGACGAGCCT GACGAAGCGA AGGATGATTC CAGGGTTGGT | |
| 1321 | GCAGATGATG CTGGTTCTTT TGAACATTAT GGGGACAATG ATTCTGGGCC TTTGGCCGGG | |
| 1381 | GAGAATGTTA TGAACGTGAT CGTGGTGGCT GCTGAATGTT CTCCATGGTG CAAAACAGGT | |
| 1441 | GGTCTTGGAG ATGTTGTGGG AGCTTTACCC AAGGCTTTAG CGAGAAGAGG ACATCGTGTT | |
| 1501 | ATGGTTGTGG TACCAAGGTA TGGGGACTAT GTGGAAGCCT TTGATATGGG AATCCGGAAA | |
| 1561 | TACTACAAAG CTGCAGGACA GGACCTAGAA GTGAACTATT TCCATGCATT TATTGATGGA | |
| 1621 | GTCGACTTTG TGTTCATTGA TGCCCCTCTT TTCCGGCACC GTCAAGATGA CATATATGGG | |
| 1681 | GGAAGTAGGC AGGAAATCAT GAAGCGCATG ATTTTGTTTT GCAAGGTTGC TGTTGAGGTT | |
| 1741 | CCTTGGCACG TTCCATGCGG TGGTGTGTGC TACGGAGATG GAAATTTGGT GTTCATTGCC | |
| 1801 | AATGATTGGC ACACTGCACT CCTGCCTGTT TATCTGAAGG CATATTACAG AGACCATGGG | |
| 1861 | TTAATGCAGT ACACTCGCTC CGTCCTCGTC ATACATAACA TCGCCCACCA GGGCCGTGGT | |
| 1921 | CCTGTAGATG AATTCCCGTA CATGGACTTG CCTGAACACT ACCTTCAACA TTTCGAGCTG | |
| 1981 | TACGATCCCG TCGGTGGCGA GCACGCCAAC ATCTTTGCCG CGGGTCTGAA GATGGCAGAC | |
| 2041 | CGGGTGGTGA CTGTCAGCCG CGGCTACCTG TGGGAGCTGA AGACAGTGGA AGGCGGCTGG | |
| 2101 | GGCCTCCACG ACATCATCCG TTCTAACGAC TGGAAGATCA ATGGCATCGT GAACGGCATC | |
| 2161 | GACCACCAGG AGTGGAACCC CAAGGTGGAC GTGCACCTGC GGTCGGACGG CTACACCAAC | |
| 2221 | TACTCCCTCG AGACACTCGA CGCTGGAAAG CGGCAGTGCA AGGCGGCCCT GCAGCGGGAG | |
| 2281 | CTGGGCCTGG AAGTGCGCGA CGACGTGCCG CTGCTCGGCT TCATCGGGCG TCTGGATGGA | |
| 2341 | CAGAAGGGCG TGGACATCAT CGGGGACGCG ATGCCGTGGA TCGCGGGGCA GGACGTGCAG | |
| 2401 | CTGGTGATGC TGGGCACCGG GCGCGCCGAC CTGGAACGAA TGCTGCAGCA CTTGGAGCGG | |
| 2461 | GAGCATCCCA ACAAGGTGCG CGGGTGGGTC GGGTTCTCGG TGCCTATGGC GCATCGCATC | |
| 2521 | ACGGCGGGCG CCGACGTGCT GGTGATGCCT TCCCGCTTCG AGCCCTGCGG GCTGAACCAG | |
| 2581 | CTCTACGCGA TGGCGTACGG CACCGTCCCT GTGGTGCACG CCGTGGGCGG GCTCAGGGAC | |
| 2641 | ACCGTGGCGC CGTTCGACCC GTTCGGCGAC GCCGGGCTCG GGTGGACTTT TGACCGCGCC | |
| 2701 | GAGGCCAACA AGCTGATCGA GGCGCTCAGG CACTGCCTCG ACACGTACCG GAAGTACGGG | |
| 2761 | GAGAGCTGGA AGAGTCTCCA GGCGCGCGGC ATGTCGCAGG ACCTCAGCTG GGACCACGCG | |
| 2821 | GCTGAGCTCT ACGAGGACGT CCTTGTCAAG GCCAAGTACC AGTGG | |
| Nucleotide sequence of maize SSIIb | | |
| 1 | GCGGCCGCCT GGTAGGCGCT GGTACAAGCG GAAGCAGCAG TAGCGTGAGG CATCCCCATG | Seq ID. No. 1144 |
| 61 | CCGGGGGCAA TCTCTTCCTC GTCGTCGGCT TTTCTCCTCC CCGTCGCGTC CTCCTCGCCG | |
| 121 | CGGCGCAGGC GGGGCAGTGT GGGTGCTGCT CTGCGCTCGT ACGGCTACAG CGGCGCGGAG | |
| 181 | CTGCGGTTGC ATTGGGCGCG GCGGGGCCCG CCTCAGGATG GAGCGGCGTC GGTACGCGCC | |
| 241 | GCAGCGGCAC CGGCCGGGGG CGAAAGCGAG GAGGCAGCGA AGAGCTCCTC CTGGTCCCAG | |
| 301 | GCGGGCGCTG TTCAGGGCAG CACGGCCAAG GCTGTGGATT CTGCTTCACC TCCCAATCCT | |
| 361 | TTGACATCTG CTCCGAAGCA AAGTCAGAGC GCTGCAATGC AAAACGGAAC GAGTGGGGGC | |
| 421 | AGCAGCGCGA GCACCGCCGC GCCGGTGTCC GGACCCAAAG CTGATCATCC ATCAGCTCCT | |
| 481 | GTCACCAAGA GAGAAATCGA TGCCAGTGCG GTGAAGCCAG AGCCCGCAGG TGATGATGCT | |
| 541 | AGACCGGTGG AAAGCATAGG CATCGCTGAA CCGGTGGATG CTAAGGCTGA TGCAGCTCCG | |
| 601 | GCTACAGATG CGGCGGCGAG TGCTCCTTAT GACAGGGAGG ATAATGAACC TGGCCCTTTG | |
| 661 | GCTGGGCCTA ATGTGATGAA CGTCGTCGTG GTGGCTTCTG AATGTGCTCC TTTCTGCAAG | |
| 721 | ACAGGTGGCC TTGGAGATGT CGTGGGTGCT TTGCCTAAGG CTCTGGCGAG GAGAGGACAC | |
| 781 | CGTGTTATGG TCGTGATACC AAGATATGGA GAGTATGCCG AAGCCCGGGA TTTAGGTGTA | |
| 841 | AGGAGACGTT ACAAGGTAGC TGGACAGGAT TCAGAAGTTA CTTATTTTCA CTCTTACATT | |
| 901 | GATGGAGTTG ATTTTGTATT CGTAGAAGCC CCTCCCTTCC GGCACCGGCA CAATAATATT | |
| 961 | TATGGGGGAG AAAGATTGGA TATTTTGAAG CGCATGATTT TGTTCTGCAA GGCCGCTGTT | |
| 1021 | GAGGTTCCAT GGTATGCTCC ATGTGGCGGT ACTGTCTATG GTGATGGCAA CTTAGTTTTC | |
| 1081 | ATTGCTAATG ATTGGCATAC CGCACTTCTG CCTGTCTATC TAAAGGCCTA TTACCGGGAC | |
| 1041 | AATGGTTTGA TGCAGTATGC TCGCTCTGTG CTTGTGATAC ACAACATTGC TCATCAGGGT | |
| 1201 | CGTGGCCCTG TAGACGACTT CGTCAATTTT GACTTGCCTG AACACTACAT CGACCACTTC | |
| 1261 | AAACTGTATG ACAACATTGG TGGGGATCAC AGCAACGTTT TTGCTGCGGG GCTGAAGACG | |
| 1321 | GCAGACCGGG TGGTGACCGT TAGCAATGGC TACATGTGGG AGCTGAAGAC TTCGGAAGGC | |
| 1381 | GGGTGGGGCC TCCACGACAT CATAAACCAG AACGACTGGA AGCTGCAGGG CATCGTGAAC | |
| 1441 | GGCATCGACA TGAGCGAGTG GAACCCCGCT GTGGACGTGC ACCTCCACTC CGACGACTAC | |
| 1501 | ACCAACTACA CGTTCGAGAC GCTGGACACC GGCAAGCGGC AGTGCAAGGC CGCCCTGCAG | |
| 1561 | CGGCAGCTGG GCCTGCAGGT CCGCGACGAC GTGCCACTGA TCGGGTTCAT CGGGCGGCTG | |
| 1621 | GACCACCAGA AGGGCGTGGA CATCATCGCC GACGCGATCC ACTGGATCGC GGGGCAGGAC | |
| 1681 | GTGCAGCTCG TGATGCTGGG CACCGGGCGG GCCGACCTGG AGGACATGCT GCGGCGGTTC | |
| 1741 | GAGTCGGAGC ACAGCGACAA GGTGCGCGCG TGGGTGGGGT TCTCGGTGCC CCTGGCGCAC | |
| 1801 | CGCATCACGG CGGGCGCGGA CATCCTGCTG ATGCCGTCGC GGTTCGAGCC GTGCGGGCTG | |
| 1861 | AACCAGCTCT ACGCCATGGC GTACGGGACC GTGCCCGTGG TGCACGCCGT GGGGGGGCTC | |
| 1921 | CGGGACACGG TGGCGCCGTT CGACCCGTTC AACGACACCG GGCTCGGGTG GACGTTCGAC | |
| 1981 | CGCGCGGAGG CGAACCGGAT GATCGACGCG CTCTCGCACT GCCTCACCAC GTACCGGAAC | |
| 2041 | TACAAGGAGA GCTGGCGCGC CTGCAGGGCG CGCGGCATGG CCGAGGACCT CAGCTGGGAC | |
| 2101 | CACGCCGCCG TGCTGTATGA GGACGTGCTC GTCAAGGCGA AGTACCAGTG GTGAGCGAAT | |
| 2161 | TAATTGGCGA CGCGACGCCG CTCCTGTCGC AGGACCTGGA CGTTATTTAG AAGGCTCTTC | |
| 2221 | TCCCTGGCGG CTTTGATGCG TGCGTCGCAT TTGCGCCGGG CGGACGGGCG ACGGTGGTTG | |
| 2281 | GCCTACCGCC TACGTCGGCT GCGTGCCCTG GGAATTTGGG CGGGCACGAT GATGCCACTG | |
| 2341 | GGCACCGGGC GCGGGGTAGT ATGATATGAA ACCGACGGCG ATGGAGATGA GGCGCATGGC | |
| 2401 | ATTTTCCCAC TGATAAATGG GGAGTTGTAT GCTACTTTAA TATCGCCACT CCTGTTAGTA | |
| 2461 | TTTATATTGA TGGCGGCCGC | |
| Nucleotide sequence of maize Du1 | | |
| 1 | GAATTCCCTA GTTCAGAGAA AGAAAGAAGT TGAGAATGAG AAGCAAGTGA GGCGCGTTTG | Seq ID. No. 1145 |
| 61 | CTGGGAAGTG GTTCTTGTGA GGTTTAGGAG TTCACCCTTT TTTTCTTCCC CTTCTAGAAA | |
| 121 | TGGAGATGGT CCTACGGTCG CAGAGCCCTC TCTGCCTTCG GAGTGGGCCG GTGCTCATTT | |
| 181 | TTCGACCAAC CGTCGCGGGC GGAGGAGGOG GCACTCAGTC TTTGTTGAGG ACTACCAGAT | |
| 241 | TTGCGAGAAG AAGGGTCATT CGATGCGTTG TAGCAAGTCC AGGTTGTCCT AATAGGAAAT | |
| 301 | CTAGGACAGC GTCTCCCAAC GTAAAAGTAG CTGCTTATAG CAACTATGCG CCAAGACTCC | |
| 361 | TCGTTGAGTC AAGCTCCAAG AAGAGCGAAC ACCATGATAG CAGCAGACAC CGTGAAGAAA | |
| 421 | CTATTGATAC ATACAATGQG CTGTCAGGTT CTGATGCAGC AGAATTGACA AGTAATAGAG | |
| 481 | ATGTAGAAAT TGAAGTGGAT TTGCAGCACA TTTCTGAGGA GGAATTGCCA GGAAAAGTAT | |
| 541 | CGATTAATGC ATCATTAGGA GAAATGGAAA CAGTGGATGA AGCTGAGGTC GAGGAGGATA | |
| 601 | AGTTTGAGGT AGATACCTCA GGAATTGTAT TGCGCAATGT TGCAGTTCGG GAAGTGGATC | |
| 661 | CAAAGGATGA ACATAATGCT AAAGATGTAT TTGTGGTAGA TTCGTCAGGA ACTGCACCAG | |
| 721 | ATAATGCTGC AGTGGAGGAA GTGGTAGATG AAGCTGAGGT TGAAGAGGAT ATGGTTGATG | |
| 781 | TGGATATCTT GGGACTTGAC TTGAATAATG CAACGATCGA GGAAATTGAT TTGATGGAAG | |
| 841 | AGGCTTTACT GGAGAACTTC GACGTGGATT CACCAGGCAA TGCTTCTAGT GGTCGAACCT | |
| 901 | ATGGGGGTGT GGATGAGTTG GGTGAGCTGC CTTCAACATC CGTGGATTGC ATCGCCATTA | |
| 961 | ACGGAAAACG TAGAAGTTTG AAGCCTAAGC CCTTGCCAAT TGTCAGGTTC CAGGAACAAG | |
| 1021 | AACAGATAGT TTTAAGCATT GTTGACGAAG AAGGGTTGAT TGCTAGTTCA TGTGAAGAAG | |
| 1081 | GCCAACCGGT GGTAGATTAC GATAAGCAAG AGGAAAACTC TACCGCTTTC GATGAACAGA | |
| 1041 | AGCAATTAAC TGATGATTTC CCTGAAGAAG GCATATCTAT AGTTCACTTC CCTGAGCCAA | |
| 1201 | ACAATGATAT TGTTGGATCC TCAAAATTCT TGGAGCAAAA ACAAGAATTG GATGGTTCTT | |
| 1261 | ATAAACAAGA TCGATCAACC ACTGGATTGC ATGAACAAGA TCAGTCTGTT GTTAGTTCAC | |
| 1321 | ACGGACAAGA TAAATCAATT GTTGGTGTGC CTCAGCAAAT CCAGTACAAT GATCAATCTA | |
| 1381 | TTGCTGGTTC TCATAGACAA GATCAATCAA TTGCCGGTGC ACCTGAGCAA ATCCAATCCG | |
| 1441 | TTGCTGGCTA TATAAAACCA AATCAATCTA TTGTTGGTTC TTGTAAACAA CATGAATTGA | |
| 1501 | TTATTCCTGA GCCTAAGAAA ATCGAATCCA TCATCAGTTA CAATGAAATA GATCAATCTA | |
| 1561 | TTGTTGGTTC TCACAAACAA GACAAATCTG TTGTTAGTGT GCCTGAGCAA ATCCAATCCA | |
| 1621 | TTGTTAGTCA CAGCAAACCA AATCAATCTA CTGTTGATTC TTATAGACAA GCTGAATCAA | |
| 1681 | TTATTGGTGT GCCTGAGAAA GTCCAATCCA TCACCAGTTA CGATAAACTA GACCAGTCCA | |
| 1741 | TTGTTGGTTC TCTTAAACAA GATGAGCCTA TTATTAGCGT GCCTGAGAAA ATCCAATCCA | |
| 1801 | TTGTCCATTA CACTAAACCA AATCAGTCTA TTGTTGGCTT GCCCAAACAA CAACAATCAA | |
| 1861 | TTGTTCATAT CGTTGAACCA AAACAGTCCA TAGATGGTTT CCCTAAACAA GATCTATCAA | |
| 1921 | TCGTTGGTAT CTCCAATGAG TTTCAAACAA AGCAACTGGC TACTGTTGGG ACTCATGATG | |
| 1981 | GATTGCTTAT GAAGGGTGTG GAAGCTAAGG AGACATCTCA AAAGACTGAA GGGGATACAC | |
| 2041 | TTCAGGCAAC GTTCAATGTC GACAACTTGT CACAGAAACA GGAAGGCTTA ACTAAAGAAG | |
| 2101 | CAGACGAGAT AACAATTATT GAGAAAATCA ATGATGAAGA CCTTGTGATG ATTGAAGAAC | |
| 2161 | AGAAAAGCAT AGCCATGAAT GAAGAACAGA CGATTGTTAC CGAAGAAGAC ATTCCAATGG | |
| 2221 | CTAAGGTTGA GATAGGAATT GACAAGGCCA AATTTTTACA TCTGCTTTCT GAAGAAGAGA | |
| 2281 | GTTCATGGGA TGAAAATGAA GTGGGAATAA TTGAGGCTGA TGAACAGTAT GAAGTCGATG | |
| 2341 | AGACATCTAT GTCCACTGAA CAAGATATCC AGGAATCACC TAATGATGAT TTGGATCCAC | |
| 2401 | AAGCACTATG GAGTATGCTT CAAGAGCTTG CTGAAAAAAA TTATTCGCTG GGAAACAAGT | |
| 2461 | TGTTTACTTA TCCAGATGTA TTGAAAGCTG ATTCAACAAT TGATCTCTAT TTCAATCGTG | |
| 2521 | ATCTATCAGC TGTGGCCAAT GAGCCTGATG TACTTATCAA AGGAGCATTC AATGGGTGGA | |
| 2581 | AGTGGAGATT TTTCACTGAA AAATTGCACA AGAGCGAGCT GGCAGGGGAC TGGTGGTGCT | |
| 2641 | GCAAACTATA CATTCCTAAG CAGGCATACA GAATGGACTT TGTGTTTTTT AACGGACACA | |
| 2701 | CGGTATATGA AAATAATAAC AATAATGATT TCGTGATACA AATAGAAAGC ACCATGGATG | |
| 2761 | AAAATTTATT TGAGGATTTC TTGGCTGAAG AAAAGCAACG AGAACTTGAG AACCTTGCAA | |
| 2821 | ATGAGGAAGC TGAAAGGAGG AGACAAACTG ATGAGCAGCG GCGAATGGAG GAAGAAAGGG | |
| 2881 | CCGCAGATAA AGCTGACAGG GTACAAGCCA AGGTTGAGGT AGAGACGAAG AAGAATAAAT | |
| 2941 | TGTGCAATGT ATTGGGTTTA GCCAGAGCTC CTGTTGATAA TTTATGGTAC ATTGAGCCCA | |
| 3001 | TCACGACTGG ACAAGAGGCT ACTGTCAGAT TGTATTATAA CATAAACTCA AGACCTCTAG | |
| 3061 | TTCACAGTAC TGAGATATGG ATGCATGGTG GCTATAACAA TTGGATTGAT GGACTCTCTT | |
| 3121 | TTGCTGAAAG GCTTGTTCAT CATCATGACA AAGATTGTGA TTGGTGGTTT GCAGATGTTG | |
| 3181 | TCGTGCCTGA AAGAACATAT GTATTGGACT GGGTTTTTGC TGACGGCCCA CCAGGGAGTG | |
| 3241 | CAAGGAATTA TGACAACAAT GGAGGACATG ATTTTCATGC TACCCTTCCA AATAACATGA | |
| 3301 | CTGAGGAAGA GTATTGGATG GAAGAAGAAC AAAGGATCTA TACAAGGCTT CAACAAGAGA | |
| 3361 | GGAGGGAAAG GGAGGAGGCT ATTAAAAGGA AGGCTGAGAG AAATGCAAAA ATGAAAGCTG | |
| 3421 | AGATGAAGGA AAAGACTATG AGAATGTTCC TGGTTTCTCA GAAACACATT GTTTACACCG | |
| 3481 | AACCACTTGA AATACATGCT GGAACTACTA TTGATGTGCT TTATAATCCT TCTAATACAG | |
| 3541 | TTCTAACTGG AAAGCCAGAG GTTTGGTTTC GATGTTCCTT TAATCGTTGG ATGTATCCAG | |
| 3601 | GTGGGGTGTT GCCACCTCAG AAGATGGTAC AAGCAGAAAA TGGTTCACAC CTAAAAGCAA | |
| 3661 | CAGTTTACGT TCCACGAGAT GCCTATATGA TGGACTTCGT TTTCTCGGAG TCAGAAGAAG | |
| 3721 | GTGGAATTTA TGATAACAGA AATGGGTTAG ACTATCATAT TCCTGTTTTT GGGTCAATTG | |
| 3781 | CAAAGGAACC ACCTATGCAC ATTGTCCACA TTGCTGTTGA GATGGCACCA ATCGCAAAGG | |
| 3841 | TTGGAGGTCT TGGTGATGTT GTCACTAGTC TTTCACGTGC TGTGCAAGAT TTAGGACACA | |
| 3901 | ATGTGGAGGT TATTCTTCCA AAGTACGGTT GCTTGAATCT AAGCAATGTC AAGAATCTAC | |
| 3961 | AAATCCATCA GAGTTTTTCT TGGGGTGGTT CTGAAATAAA TGTGTGGCGT GGACTAGTCG | |
| 4021 | AAGGCCTTTG TGTTTACTTC CTGGAACCTC AAAATGGGAT GTTTGGAGTC GGATATGTAT | |
| 4081 | ATGGCAGGGA CGATGACCGC CGATTTGGCT TCTTCTGTCG TTCTGCTCTA GAGTTTCTCC | |
| 4141 | TCCAAAGTGG ATCTTCTCCG AACATAATAC ATTGCCATGA TTGGTCAAGT GCTCCTGTTG | |
| 4201 | CCTGGCTACA CAAGGAAAAC TACGCGAAGT CTAGCTTGGC AAACGCACGG GTGGTATTCA | |
| 4261 | CCATCCACAA TCTTGAATTT GGAGCGCATC ATATTGGCAA AGCAATGAGA TATTGTGATA | |
| 4321 | AAGCAACAAC TGTCTCTAAT ACATATTCAA AGGAAGTGTC AGGTCATGGT GCCATAGTTC | |
| 4381 | CTCATCTTGG GAAATTCTAT GGCATTCTCA ATGGAATTGA TCCGGATATA TGGGATCCGT | |
| 4441 | ACAATGACAA CTTTATCCCG GTCCACTACA CTTGTGAGAA TGTGGTTGAA GGCAAGAGGG | |
| 4501 | CTGCTAAGAG GGCACTGCAG CAGAAGTTTG GGTTACAGCA AATCGATGTC CCCGTCGTAG | |
| 4561 | GAATCGTCAC TCGCCTGACA GCCCAAAAGG GGATCCACCT GATCAAGCAT GCGATTCACC | |
| 4621 | GTACACTCGA ACGGAACGGA CAGGTGGTTT TGCTTGGTTC AGCGCCGGAC TCTCGAATCC | |
| 4681 | AAGCTGATTT TGTCAACCTG GCGAATACGC TCCACGGCGT AAACCATGGG CAAGTGAGGC | |
| 4741 | TTTCCTTGAC CTACGACGAG CCTCTCTCGC ATCTGATATA CGCTGGCTCT GACTTCATTC | |
| 4801 | TGGTCCCATC TATATTTGAG CCTTGCGGCC TAACTCAGCT CGTCGCCATG CGGTATGGAA | |
| 4861 | CCATCCCGAT TGTCCGCAAG ACTGGAGGGC TCTTCGACAC TGTCTTCGAT GTGGACAATG | |
| 4921 | ACAAGGAACG AGCCCGAGAT CGAGGCCTTG AGCCCAACGG GTTTAGCTTT GACGGAGCTG | |
| 4981 | ATAGCAACGG TGTTGACTAC GCGCTGAACA GGGCGATCTC AGCTTGGTTC GATGCCCGGA | |
| 5041 | GCTGGTTCCA CTCCCTTTGC AAGAGAGTCA TGGAGGAGGA CTGGTCGTGG AACCGACCTG | |
| 5101 | CCCTCGACTA CATCGAGCTC TACCGTTCAG CGTCCAAATT GTAATAATCC AAACAACGGC | |
| 5161 | CAATGTAGTG TGTTGTCTGC AGGTCTCAGA TGCAGCCATT CAGCTTTTGC AGGTTCCTGG | |
| 5221 | GCATTGCTGT ACAGCCTCCT TGTCTTTAGT TAGCTCCATT CCCCGAGGAG CACAGTGCAA | |
| 5281 | TTTTTTATCC TCAGTTATTA TGCATAGATT GTCTCAGTAG AATGCTTTCT TCGGGCATGT | |
| 5341 | ATGTTTGTTT CCTCTGTTGT TGAATTCTGG TGTTAAGTCG CGTATAGGAA TCTACAGGAA | |
| 5401 | ATGAAAAAGT CCATTTCCTG CGTCAACCTT TTAGGGCTAC CATGCACATG AGACCTTTCA | |
| 5461 | AGTGCAAAGA ATATTAGGAC TAGACTACTA GTATGTGAAC TCTATTTTTC CAAGAGATTT | |
| 5521 | CAATTTTTCC AATGAAAAAT AAACTAATTT TTCTTGGAAA AATGGAAATC CCTTGGAAAA | |
| 5581 | ATGGGGTTCC CAAACTAGCC CGTAGAGTAT AGATCATAGA ATTGGTCTAG TGGTTCCTCG | |
| 5641 | AGAGAGAAAA AAACATAGAC TTTTCTTGTC ATATGCTTAT TTAAGTTTAT TTTGTACAAA | |
| 5701 | CTTTGAGAAC CTTCAAAAAC ACCCCAATGG CTGGTTAAGT GACCAGGGAA ATAAAGAGGA | |
| 5761 | TCTATAGGGA GGAATCCCCC GCCTCTCTCT CACAGATGTT GCCTAGCACC GGCCAGCCTC | |
| 5821 | ATCCGTCCAG TGGAATTAAG GTTGGTTGCG ACGACAGCCC ATCAATGGAA ACCAACCTCG | |
| 5881 | TGCCCCGTGC CGGGATCTAC CTTCCTTCCT CACCACCACG CCGATCTCAC CTTCCATAGG | |
| 5941 | AGCTTCCTAT GCACTGTTAC CTATTATAGG TACATGACAT TGTACATCTT TGTATGAACT | |
| 6001 | TACATCAATG CCAAAAATCC GGAATTC | |

| | |
|---|---|
| C-terminal end (“CTEND”) of maize GBSS; | |
| K V V G T P A Y E E M V R N C M I Q D L S W K G P A K N W E N V L L S L G V A | Seq ID. No. 1146 |
| G G E P G V E G E E I A P L A K E N V A A P | |
| C-terminal end (“CTEND”) of maize SSI; | |
| G E Q G T G W A F A P L T T E N M F V D I A N C N I Y I Q G T | Seq ID. No. 1147 |
| Q V L L G R A N E A R H V K R L H V G P C R | |
| C-terminal end (“CTEND”) of maize SSIIa; | |
| H C L D T Y R K Y G E S W K S L Q A R G M S Q D L S W D H A A E L | Seq ID. No. 1148 |
| Y E D V L V K A K Y Q W | |
| C-terminal end (“CTEND”) of maize SSIIb; | |
| N R M I D A L S H C L T T Y R N Y K E S W R A C R A R G M A E D L S W D H A A | Seq ID. No. 1149 |
| V L Y E D V L V K A K Y Q W | |
| C-terminal end (“CTEND”) of maize SSIII (Du-1); | |
| N D K E R A R D R G L E P N G F S F D G A D S N G V D Y A L N R A I S A W F D | Seq ID. No. 1150 |
| A R S W F H S L C K R V M E Q D W S W N R P A L D Y I E L Y R S A S K L | |

TABLE XXXVII
List of Glucan Transferases with Different Database Accession Identifications

| | |
|---|---|
| 1: | NP_385953 1. PHOSPHOGLYCEROL TRANSFERASE, CYCLIC BETA-1,2-GLUCAN MODIFICATION PROTEIN |
| | [Sinorhizobium meliloti] |
| | gi\|15965600\|ref\|NP_385953.1\|[15965600] |
| 2: | Q9SD76 |
| | Alpha-glucan phosphorylase, H isozyme (Starch phosphorylase H) |
| | gi\|14916634\|sp\|Q9SD76\|PHSH_ARATH[14916634] |
| 3: | Q9LKJ3 |
| | Alpha-glucan phosphorylase, H isozyme (Starch phosphorylase H) |
| | gi\|14916632\|sp\|Q9LKJ3\|PHSH_WHEAT[14916632] |
| 4: | Q9PKU9 |
| | 4-alpha-glucanotransferase (Amylomaltase) (Disproportionating enzyme) (D-enzyme) |
| | gi\|14194917\|sp\|Q9PKU9\|MALQ_CHLMU[14194917] |
| 5: | Q9Z1E4 |
| | Glycogen [starch] synthase, muscle |
| | gi\|12230199\|sp\|Q9Z1E4\|GYS1_MOUSE[12230199] |
| 6: | Q9VFC8 |
| | Putative glycogen [starch] synthase |
| | gi\|12230129\|sp\|Q9VFC8\|GYS_DROME[12230129] |
| 7: | Q9U2D9 |
| | Putative glycogen [starch] synthase |
| | gi\|12230128\|sp\|Q9U2D9\|GYS_CAEEL[12230128] |
| 8: | Q9Y704 |
| | Cell wall alpha-1,3-glucan synthase mok14 |
| | gi\|11386958\|sp\|Q9Y704\|MOKE_SCHPO[11386958] |
| 9: | P27472 |
| | Glycogen [starch] synthase, isoform 2 |
| | gi\|1717973\|sp\|P27472\|GYS2_YEAST[1717973] |
| 10: | P54840 |
| | Glycogen [starch] synthase, liver |
| | gi\|1717972\|sp\|P54840\|GYS2_HUMAN[1717972] |
| 11: | P54859 |
| | Glycogen [starch] synthase, brain |
| | gi\|1717968\|sp\|P54859\|GYS3_MOUSE[1717968] |
| 12: | P13807 |
| | Glycogen [starch] synthase, muscle |
| | gi\|1351366\|sp\|P13807\|GYS1_HUMAN[1351366] |
| 13: | P17625 |
| | Glycogen [starch] synthase, liver |
| | gi\|136764\|sp\|P17625\|GYS2_RAT[136764] |
| 14: | P23337 |
| | Glycogen [starch] synthase, isoform 1 |
| | gi\|136753\|sp\|P23337\|GYS1_YEAST[136753] |
| 15: | P04045 |
| | Alpha-1,4 glucan phosphorylase, L-1 isozyme, chloroplast precursor (Starch phosphorylase L-1) |
| | gi\|130173\|sp\|P04045\|PHS1_SOLTU[130173] |
| 16: | P27598 |
| | Alpha-1,4 glucan phosphorylase, L isozyme, chloroplast precursor (Starch phosphorylase L) |
| | gi\|130172\|sp\|P27598\|PHSL_IPOBA[130172] |
| 17: | O93869 |
| | Glycogen [starch] synthase |
| | gi\|12230121\|sp\|O93869\|GYS_NEUCR[12230121] |
| 18: | P80099 |
| | 4-alpha-glucanotransferase (Amylomaltase) (Disproportionating enzyme) (D-enzyme) |
| | gi\|6226644\|sp\|P80099\|MGTA_THEMA[6226644] |
| 19: | O87172 |
| | 4-alpha-glucanotransferase (Amylomaltase) (Disproportionating enzyme) (D-enzyme) |
| | gi\|6225654\|sp\|O87172\|MALQ_THEAQ[6225654] |
| 20: | P72785 |
| | 4-alpha-glucanotransferase (Amylomaltase) (Disproportionating enzyme) (D-enzyme) |
| | gi\|6225653\|sp\|P72785\|MALQ_SYNY3[6225653] |
| 21: | O53932 |
| | 4-alpha-glucanotransferase (Amylomaltase) (Disproportionating enzyme) (D-enzyme) |
| | gi\|6225652\|sp\|O53932\|MALQ_MYCTU[6225652] |
| 22: | O84089 |
| | 4-alpha-glucanotransferase (Amylomaltase) (Disproportionating enzyme) (D-enzyme) |
| | gi\|6225650\|sp\|O84089\|MALQ_CHLTR[6225650] |
| 23: | O66937 |
| | 4-alpha-glucanotransferase (Amylomaltase) (Disproportionating enzyme) (D-enzyme) |
| | gi\|6225647\|sp\|O66937\|MALQ_AQUAE[6225647] |
| 24: | P13031 |
| | Glycogen phosphorylase |
| | gi\|2851413\|sp\|P13031\|PHSG_ECOLI[2851413] |
| 25: | P53536 |
| | Alpha-1,4 glucan phosphorylase, L isozyme, chloroplast precursor (Starch phosphorylase L) |
| | gi\|2506470\|sp\|P53536\|PHSL_VICFA[2506470] |
| 26: | P53535 |
| | Alpha-1,4 glucan phosphorylase, L-2 isozyme, chloroplast precursor (Starch phosphorylase L-2) |
| | gi\|1730557\|sp\|P53535\|PHS2_SOLTU[1730557] |
| 27: | P52981 |
| | 1,4-alpha-glucan branching enzyme (Glycogen branching enzyme) |
| | gi\|1707936\|sp\|P52981\|GLGB_SYNY3[1707936] |
| 28: | Q10625 |
| | Probable 1,4-alpha-glucan branching enzyme (Glycogen branching enzyme) |
| | gi\|1707934\|sp\|Q10625\|GLGB_MYCTU[1707934] |
| 29: | P45176 |
| | 4-alpha-glucanotransferase (Amylomaltase) (Disproportionating enzyme) (D-enzyme) |
| | gi\|1170869\|sp\|P45176\|MALQ_HAEIN[1170869] |
| 30: | P15977 |
| | 4-alpha-glucanotransferase (Amylomaltase) (Disproportionating enzyme) (D-enzyme) |
| | gi\|1170868\|sp\|P15977\|MALQ_ECOLI[1170868] |
| 31: | P30924 |
| | 1,4-alpha-glucan branching enzyme (Starch branching enzyme) (Q-enzyme) |
| | gi\|1169912\|sp\|P30924\|GLGB_SOLTU[1169912] |
| 32: | P45177 |
| | 1,4-alpha-glucan branching enzyme (Glycogen branching enzyme) |
| | gi\|1169910\|sp\|P45177\|GLGB_HAEIN[1169910] |
| 33: | P38678 |
| | Glucan synthase-1 (1,3-beta-glucan synthase) (UDP-glucose-1,3-beta-D-glucan glucosyltransferase) |
| | gi\|729636\|sp\|P38678\|GS1_NEUCR[729636] |
| 34: | P39118 |
| | 1,4-alpha-glucan branching enzyme (Glycogen branching enzyme) |
| | gi\|729579\|sp\|P39118\|GLGB_BACSU[729579] |
| 35: | Q04446 |
| | 1,4-alpha-glucan branching enzyme (Glycogen branching enzyme) (Brancher enzyme) |
| | gi\|544388\|sp\|Q04446\|GLGB_HUMAN[544388] |
| 36: | Q06801 |
| | 4-alpha-glucanotransferase, chloroplast precursor (Amylomaltase) (Disproportionating enzyme) (D-enzyme) |
| | gi\|544184\|sp\|Q06801\|DPEP_SOLTU[544184] |
| 37: | Q06119 |
| | Diacylglycerol kinase (DAGK) (Diglyceride kinase) (DGK) |
| | gi\|462437\|sp\|Q06119\|KDGL_RHIME[462437] |
| 38: | P32434 |
| | Type I protein geranylgeranyltransferase beta subunit (Type I protein geranyl-geranyltransferase beta subunit) (GGTase-I-beta) (PGGT) |
| | gi\|416853\|sp\|P32434\|CWG2_SCHPO[416853] |
| 39: | P29851 |
| | 4-alpha-glucanotransferase (Amylomaltase) (Disproportionating enzyme) (D-enzyme) |
| | gi\|266497\|sp\|P29851\|MALQ_STRPN[266497] |
| 40: | P07762 |
| | 1,4-alpha-glucan branching enzyme (Glycogen branching enzyme) |
| | gi\|121296\|sp\|P07762\|GLGB_ECOLI[121296] |
| 41: | O32462 |
| | 4-alpha-glucanotransferase (Amylomaltase) (Disproportionating enzyme) (D-enzyme) |
| | gi\|18202015\|sp\|O32462\|MALQ_THELI[18202015] |
| 42: | O32450 |
| | 4-alpha-glucanotransferase (Amylomaltase) (Disproportionating enzyme) (D-enzyme) |
| | gi\|18202014\|sp\|O32450\|MALQ_PYRKO[18202014] |
| 43: | Q08047 |
| | 1,4-alpha-glucan branching enzyme IIB, chloroplast precursor (Starch branching enzyme IIB) (Q-enzyme) |
| | gi\|1169911\|sp\|Q08047\|GLGB_MAIZE[1169911] |
| 44: | Q9Z6V8 |
| | Glycogen synthase (Starch [bacterial glycogen] synthase) |
| | gi\|17367290\|sp\|Q9Z6V8\|GLGA_CHLPN[17367290] |
| 45: | Q9WZZ7 |
| | Glycogen synthase (Starch [bacterial glycogen] synthase) |
| | gi\|17367208\|sp\|Q9WZZ7\|GLGA_THEMA[17367208] |
| 46: | Q9RWS1 |
| | Glycogen synthase (Starch [bacterial glycogen] synthase) |
| | gi\|17367076\|sp\|Q9RWS1\|GLGA_DEIRA[17367076] |
| 47: | Q9RNH6 |
| | Glycogen synthase (Starch [bacterial glycogen] synthase) |
| | gi\|17367070\|sp\|Q9RNH6\|GLGA_RHOSH[17367070] |
| 48: | Q9PLC3 |
| | Glycogen synthase (Starch [bacterial glycogen] synthase) |
| | gi\|17367025\|sp\|Q9PLC3\|GLGA_CHLMU[17367025] |
| 49: | Q9KRB6 |
| | Glycogen synthase (Starch [bacterial glycogen] synthase) |
| | gi\|17366937\|sp\|Q9KRB6\|GLGA_VIBCH[17366937] |
| 50: | Q9KDX6 |
| | Glycogen synthase (Starch [bacterial glycogen] synthase) |
| | gi\|17366934\|sp\|Q9KDX6\|GLGA_BACHD[17366934] |
| 51: | Q9I1V0 |
| | Glycogen synthase (Starch [bacterial glycogen] synthase) |
| | gi\|17366864\|sp\|Q9I1V0\|GLGA_PSEAE[17366864] |
| 52: | Q9EUT5 |
| | Glycogen synthase (Starch [bacterial glycogen] synthase) |
| | gi\|17366749\|sp\|Q9EUT5\|GLGA_RHITR[17366749] |
| 53: | Q9CN91 |
| | Glycogen synthase (Starch [bacterial glycogen] synthase) |
| | gi\|17366721\|sp\|Q9CN91\|GLGA_PASMU[17366721] |
| 54: | Q9CHM9 |
| | Glycogen synthase (Starch [bacterial glycogen] synthase) |
| | gi\|17366711\|sp\|Q9CHM9\|GLGA_LACLA[17366711] |
| 55: | Q985P2 |
| | Glycogen synthase (Starch [bacterial glycogen] synthase) |
| | gi\|17366611\|sp\|Q985P2\|GLGA_RHILO[17366611] |
| 56: | Q97QS5 |
| | Glycogen synthase (Starch [bacterial glycogen] synthase) |
| | gi\|17366608\|sp\|Q97QS5\|GLGA_STRPN[17366608] |
| 57: | Q97GX6 |
| | Glycogen synthase (Starch [bacterial glycogen] synthase) |
| | gi\|17366595\|sp\|Q97GX6\|GLGA_CLOAB[17366595] |
| 58: | P72623 |
| | Probable glycogen synthase 2 (Starch [bacterial glycogen] synthase 2) |
| | gi\|17366372\|sp\|P72623\|GLG2_SYNY3[17366372] |
| 59: | P58395 |
| | Glycogen synthase (Starch [bacterial glycogen] synthase) |
| | gi\|17366350\|sp\|P58395\|GLGA_THECA[17366350] |
| 60: | P58394 |
| | Glycogen synthase 2 (Starch [bacterial glycogen] synthase 2) |
| | gi\|17366347\|sp\|P58394\|GLG2_RHIME[17366347] |
| 61: | P58393 |
| | Glycogen synthase 1 (Starch [bacterial glycogen] synthase 1) |
| | gi\|17366344\|sp\|P58393\|GLG1_RHIME[17366344] |
| 62: | O84804 |
| | Glycogen synthase (Starch [bacterial glycogen] synthase) |
| | gi\|17366221\|sp\|O84804\|GLGA_CHLTR[17366221] |
| 63: | O66935 |
| | Glycogen synthase (Starch [bacterial glycogen] synthase) |
| | gi\|17366197\|sp\|O66935\|GLGA_AQUAE[17366197] |
| 64: | P35573 |
| | Glycogen debranching enzyme (Glycogen debrancher) [Includes: 4-alpha-glucanotransferase (Oligo-1,4-1,4-glucantransferase); Amylo-alpha-1,6- |
| | glucosidase (Amylo-1,6-glucosidase) (Dextrin 6-alpha-D-glucosidase)] |
| | gi\|8928542\|sp\|P35573\|GDE_HUMAN[8928542] |
| 65: | Q59001 |
| | Probable glycogen synthase (Starch [bacterial glycogen] synthase) |
| | gi\|2842612\|sp\|Q59001\|GLGA_METJA[2842612] |
| 66: | P74521 |
| | Glycogen synthase (Starch [bacterial glycogen] synthase) |
| | gi\|2829618\|sp\|P74521\|GLGA_SYNY3[2829618] |
| 67: | O08328 |
| | Glycogen synthase (Starch [bacterial glycogen] synthase) |
| | gi\|2811062\|sp\|O08328\|GLGA_BACST[2811062] |
| 68: | P45179 |
| | Glycogen synthase (Starch [bacterial glycogen] synthase) |
| | gi\|1169909\|sp\|P45179\|GLGA_HAEIN[1169909] |
| 69: | P08323 |
| | Glycogen synthase (Starch [bacterial glycogen] synthase) |
| | gi\|1169908\|sp\|P08323\|GLGA_ECOLI[1169908] |
| 70: | P39125 |
| | Glycogen synthase (Starch [bacterial glycogen] synthase) |
| | gi\|729578\|sp\|P39125\|GLGA_BACSU[729578] |
| 71: | P39670 |
| | Glycogen synthase (Starch [bacterial glycogen] synthase) |
| | gi\|729577\|sp\|P39670\|GLGA_AGRTU[729577] |
| 72: | P35574 |
| | Glycogen debranching enzyme (Glycogen debrancher) [Includes: 4-alpha-glucanotransferase (Oligo-1,4-1,4-glucantransferase); Amylo-alpha-1,6- |
| | glucosidase (Amylo-1,6-glucosidase) (Dextrin 6-alpha-D-glucosidase)] |
| | gi\|544379\|sp\|P35574\|GDE_RABIT[544379] |
| 73: | P05416 |
| | Glycogen synthase (Starch [bacterial glycogen] synthase) (Fragment) |
| | gi\|121295\|sp\|P05416\|GLGA_SALTY[121295] |
| 74: | 1G5AA |
| | Chain A, Amylosucrase From Neisseria Polysaccharea |
| | gi\|16974797\|pdb\|1G5A\|A[16974797] |
| 75: | B26206 |
| | alpha-1,4-glucan-protein synthase (UDP-forming) (EC 2.4.1.112) - rabbit (fragment) |
| | gi\|89923\|pir\|\|B26206[89923] |
| 76: | P30538 |
| | 1,4-ALPHA-GLUCAN BRANCHING ENZYME (GLYCOGEN BRANCHING ENZYME) |
| | gi\|232168\|sp\|P30538\|GLGB_BACST[232168] |
| 77: | Q9UUL4 |
| | CELL WALL ALPHA-1,3-GLUCAN SYNTHASE MOK12 |
| | gi\|11386953\|sp\|Q9UUL4\|MOKC_SCHPO[11386953] |
| 78: | CAC46426 |
| | PHOSPHOGLYCEROL TRANSFERASE, CYCLIC BETA-1,2-GLUCAN MODIFICATION PROTEIN [Sinorhizobium meliloti] |
| | gi\|15074781\|emb\|CAC46426.1\|[15074781] |
| 79: | Q09854 |
| | CELL WALL ALPHA-1,3-GLUCAN SYNTHASE MOK11 |
| | gi\|12644399\|sp\|Q09854\|MOKB_SCHPO[12644399] |
| 80: | P13834 |
| | GLYCOGEN [STARCH] SYNTHASE, MUSCLE |
| | gi\|12644124\|sp\|P13834\|GYS1_RABIT[12644124] |
| 81: | P10249 |
| | SUCROSE PHOSPHORYLASE (SUCROSE GLUCOSYLTRANSFERASE) (GLUCOSYLTRANSFERASE-A) (GTF-A) |
| | gi\|121726\|sp\|P10249\|SUCP_STRMU[121726] |
| 82: | Q9Y719 |
| | CELL WALL ALPHA-1,3-GLUCAN SYNTHASE MOK13 |
| | gi\|12643961\|sp\|Q9Y719\|MOKD_SCHPO[12643961] |
| 83: | Q9USK8 |
| | CELL WALL ALPHA-1,3-GLUCAN SYNTHASE MOK1 |
| | gi\|12643908\|sp\|Q9USK8\|MOK1_SCHPO[12643908] |
| 84: | Q9Z8L2 |
| | 4-ALPHA-GLUCANOTRANSFERASE (AMYLOMALTASE) (DISPROPORTIONATING ENZYME) (D-ENZYME) |
| | gi\|6225648\|sp\|Q9Z8L2\|MALQ_CHLPN[6225648] |
| 85: | S41686 |
| | geranylgeranyltransferase type I (EC 2.5.1.-) beta chain - fission yeast (Schizosaccharomyces pombe) |
| | gi\|542213\|pir\|\|S41686[542213] |
| 86: | O86956 |
| | 4-ALPHA-GLUCANOTRANSFERASE (AMYLOMALTASE) (DISPROPORTIONATING ENZYME) (D-ENZYME) |
| | gi\|6225664\|sp\|O86956\|MGTA_THENE[6225664] |
| 87: | Q59266 |
| | 4-ALPHA-GLUCANOTRANSFERASE (AMYLOMALTASE) (DISPROPORTIONATING ENZYME) (D-ENZYME) |
| | gi\|6225651\|sp\|Q59266\|MALQ_CLOBU[6225651] |
| 88: | O34022 |
| | 4-ALPHA-GLUCANOTRANSFERASE (AMYLOMALTASE) (DISPROPORTIONATING ENZYME) (D-ENZYME) |
| | gi\|6225649\|sp\|O34022\|MALQ_CHLPS[6225649] |
| 89: | AAD37783 |
| | glucan synthase [Paracoccidioides brasiliensis] |
| | gi\|5007025\|gb\|AAD37783.1\|AF148715_1[5007025] |
| 90: | NQECA |
| | 1,4-alpha-glucan branching enzyme (EC 2.4.1.18) - Escherichia coli |
| | gi\|66573\|pir\|\|NQECA[66573] |
| 91: | P49331 |
| | GLUCOSYLTRANSFERASE-S PRECURSOR (GTF-S) (DEXTRANSUCRASE) (SUCROSE 6-GLUCOSYLTRANSFERASE) |
| | gi\|6166192\|sp\|P49331\|GTFD_STRMU[6166192] |
| 92: | P08987 |
| | GLUCOSYLTRANSFERASE-I PRECURSOR (GTF-I) (DEXTRANSUCRASE) (SUCROSE 6-GLUCOSYLTRANSFERASE) |
| | gi\|6166191\|sp\|P08987\|GTFB_STRMU[6166191] |
| 93: | P13470 |
| | GLUCOSYLTRANSFERASE-SI PRECURSOR (GTF-SI) (DEXTRANSUCRASE) (SUCROSE 6-GLUCOSYLTRANSFERASE) |
| | gi\|121728\|sp\|P13470\|GTFC_STRMU[121728] |
| 94: | P53537 |
| | ALPHA-GLUCAN PHOSPHORYLASE, H ISOZYME (STARCH PHOSPHORYLASE H) |
| | gi\|1730560\|sp\|P53537\|PHSH_VICFA[1730560] |
| 95: | P32811 |
| | ALPHA-GLUCAN PHOSPHORYLASE, H ISOZYME (STARCH PHOSPHORYLASE H) |
| | gi\|417488\|sp\|P32811\|PHSH_SOLTU[417488] |
| 96: | P52979 |
| | 1,4-ALPHA-GLUCAN BRANCHING ENZYME (GLYCOGEN BRANCHING ENZYME) |
| | gi\|3915711\|sp\|P52979\|GLGB_AGRTU[3915711] |
| 97: | Q04952 |
| | PUTATIVE 1,3-BETA-GLUCAN SYNTHASE COMPONENT (1,3-BETA-D-GLUCAN-UDP GLUCOSYLTRANSFERASE) |
| | gi\|2498415\|sp\|Q04952\|GLS3_YEAST[2498415] |
| 98: | P40989 |
| | 1,3-BETA-GLUCAN SYNTHASE COMPONENT GLS2 (1,3-BETA-D-GLUCAN-UDP GLUCOSYLTRANSFERASE) |
| | gi\|1707982\|sp\|P40989\|GLS2_YEAST[1707982] |
| 99: | P52980 |
| | 1,4-ALPHA-GLUCAN BRANCHING ENZYME (GLYCOGEN BRANCHING ENZYME) |
| | gi\|1707935\|sp\|P52980\|GLGB_STRAU[1707935] |
| 100: | P38631 |
| | 1,3-BETA-GLUCAN SYNTHASE COMPONENT GLS1 (1,3-BETA-D-GLUCAN-UDP GLUCOSYLTRANSFERASE) (CND1 PROTEIN) |
| | (CWN53 PROTEIN) (FKS1 PROTEIN) (PAPULACANDIN B SENSITIVITY PROTEIN 1) |
| | gi\|1346146\|sp\|P38631\|GLS1_YEAST[1346146] |
| 101: | P32775 |
| | 1,4-ALPHA-GLUCAN BRANCHING ENZYME (GLYCOGEN BRANCHING ENZYME) |
| | gi\|729580\|sp\|P32775\|GLGB_YEAST[729580] |
| 102: | Q01401 |
| | 1,4-ALPHA-GLUCAN BRANCHING ENZYME (STARCH BRANCHING ENZYME) (Q-ENZYME) |
| | gi\|399544\|sp\|Q01401\|GLGB_ORYSA[399544] |
| 103: | P30539 |
| | 1,4-ALPHA-GLUCAN BRANCHING ENZYME (GLYCOGEN BRANCHING ENZYME) |
| | gi\|232169\|sp\|P30539\|GLGB_BUTFI[232169] |
| 104: | P30537 |
| | 1,4-ALPHA-GLUCAN BRANCHING ENZYME (GLYCOGEN BRANCHING ENZYME) |
| | gi\|232167\|sp\|P30537\|GLGB_BACCL[232167] |
| 105: | P29336 |
| | GLUCOSYLTRANSFERASE-S PRECURSOR (GTF-S) (DEXTRANSUCRASE) (SUCROSE 6-GLUCOSYLTRANSFERASE) |
| | gi\|121729\|sp\|P29336\|GTFS_STRDO[121729] |
| 106: | P27470 |
| | GLUCOSYLTRANSFERASE-I PRECURSOR (GTF-I) (DEXTRANSUCRASE) (SUCROSE 6-GLUCOSYLTRANSFERASE) |
| | gi\|121725\|sp\|P27470\|GTF2_STRDO[121725] |
| 107: | P11001 |
| | GLUCOSYLTRANSFERASE-I PRECURSOR (GTF-I) (DEXTRANSUCRASE) (SUCROSE 6-GLUCOSYLTRANSFERASE) |
| | gi\|121724\|sp\|P11001\|GTF1_STRDO[121724] |
| 108: | P16954 |
| | 1,4-ALPHA-GLUCAN BRANCHING ENZYME (GLYCOGEN BRANCHING ENZYME) |
| | gi\|121297\|sp\|P16954\|GLGB_SYNP7[121297] |
| 109: | AAB41531 |
| | cyclic beta-1,2-glucan modification protein; phosphoglycerol transferase [Sinorhizobium meliloti] |
| | gi\|4733934\|gb\|AAB41531.2\|[4733934] |
| 110: | AAC62210 |
| | beta-(1-3)-glucosyl transferase [Bradyrhizobium japonicum] |
| | gi\|3687658\|gb\|AAC62210.1\|[3687658] |
| 111: | AAC61753 |
| | beta-1,3 glucan transferase Bg\|2p [Pichia jadinii] |
| | gi\|3661545\|gb\|AAC61753.1\|[3661545] |
| 112: | AAB46846 |
| | alpha-1,4-glucan orthophosphate glycosyl transferase; myophosphorylase; glycogen phosphorylase isozyme [Bos taurus] |
| | gi\|1836054\|gb\|AAB46846.1\|[1836054] |
| 113: | AAB03100 |
| | starch branching enzyme class II [Arabidopsis thaliana] |
| | gi\|726490\|gb\|AAB03100.1\|[726490] |
| 114: | AAB03099 |
| | starch branching enzyme class II [Arabidopsis thaliana] |
| | gi\|619939\|gb\|AAB03099.1\|[619939] |
| 115: | CAA78143 |
| | dimethylallyltransferase [Schizosaccharomyces pombe] |
| | gi\|396477\|emb\|CAA78143.1\|[396477] |
| 116: | AAA34632 |
| | 1,4-glucan-6-(1,4-glucano)-transferase [Saccharomyces cerevisiae] |
| | gi\|171569\|gb\|AAA34632.1\|[171569] |
| 117: | AAA23872 |
| | branching enzyme (EC 2.4.1.18) [Escherichia coli] |
| | gi\|146142\|gb\|AAA23872.1\|[146142] |
| 118: | AAA21151 |
| | beta-1,3 glucan transferase [Candida albicans] |
| | gi\|532776\|gb\|AAA21151.1\|[532776] |

We claim:

1. An isolated DNA molecule encoding a fusion protein consisting of four different functional domains selected from the group consisting of GLASS, LINKR, GLYTR, and CTEND which are operably linked to one another.

2. An isolated DNA molecule in claim 1, in which the GLASS domain comprises a GBSS GLASS.

3. The isolated DNA molecule in claim 2 wherein the GBSS GLASS comprises a GLASS of SEQ ID NO: 1

4. An isolated DNA molecule in claim 1, in which the GLASS domain comprises a SSI GLASS.

5. The isolated DNA molecule of claim 4 wherein the SSI GLASS comprises a GLASS of SEQ ID NO: 2

6. An isolated DNA molecule in claim 1, in which the GLASS domain comprises a SSII GLASS.

7. The isolated DNA molecule of claim 6 wherein the SSII GLASS comprises a GLASS of SEQ ID NOs. 3 and 4

8. An isolated DNA molecule in claim 1, in which the GLASS domain comprises a SSIII GLASS.

9. The isolated DNA molecule of claim 8 wherein the SSIII GLASS comprises a GLASS of SEQ ID NO:5

10. The isolated DNA molecule of claim 2, wherein said GBSS GLASS is a GLASS of a glucan producing organism.

11. The isolated DNA molecule of claim 4, wherein said SSI-GLASS is a GLASS domain of a glucan producing organism.

12. The isolated DNA molecule of claim 6, wherein said SSII-GLASS is a GLASS of a glucan producing organism.

13. The isolated DNA molecule of claim 8, wherein said SSIII-GLASS is a GLASS of a glucan producing organism.

14. The isolated DNA molecule of claim 2 wherein said GBSS-GLASS domain is at least 80% identical to a GLASS peptide of a glucan producing organism.

15. The isolated DNA molecule of claim 4 wherein said SSI-GLASS domain is at least 80% identical to a GLASS peptide of a glucan producing organism.

16. The isolated DNA molecule of claim 6 wherein said SSII-GLASS domain is at least 80% identical to a GLASS peptide of a glucan producing organism.

17. The isolated DNA molecule of claim 8 wherein said SSIII-GLASS domain is at least 80% identical to a GLASS peptide of a glucan producing organism.

18. An isolated DNA molecule of any of the claims in 2, 4, 6 or 8, in which the LINKR domain is a GBSS-LINKR, SSI-LINKR, SSII-LINKR or SSIII-LINKR.

19. The isolated DNA molecule of claim 18 wherein said LINKR comprises a LINKR sequence selected from SEQ ID NOs:121-171; 336-386;527-577; 733-783; and 983-1033.

20. An isolated DNA molecule of any of claims 2, 4, 6 or 8, in which the GLYTR domain is a GBSS-GLYTR, SSI-GLYTR, SSII-GLYTR or SSIII-GLYTR.

21. An isolated DNA molecule of claim 18, in which the GLYTR is a GBSS-GLYTR, SSI-GLYTR, SSII-GLYTR or SSfi-GLYTR.

22. The isolated DNA molecule of claim 20 wherein said GLYTR comprises a GLYTR sequence selected from SEQ ID NOs:1136, 1137, 1138, 1139, and 1140.

23. The isolated DNA molecule of claim 21 wherein said GLYTR comprises a GLYTR sequence selected from SEQ ID NOs: 172-222; 387-437; 578-628; 784-834; and 1034-1084.

24. An isolated DNA molecule of claims 2, 4, 6 or 8, in which the CTEND domain is a GBSS-CTEND, SSI-CTEND, SSII-CTEND or SSIII-CTEND.

25. An isolated DNA molecule of claim 18, in which the CTEND domain is a GBSS-CTEND, SSI-CTEND, SSII-CTEND or SSIII-CTEND.

26. An isolated DNA molecule of claim 20, in which the CTEND domain is a GBSS-CTEND, SSI-CTEND, SSII-CTEND or SSIII-CTEND.

27. The isolated DNA molecule of claim 24 wherein said CTEND sequence comprises a CTEND sequence selected from SEQ ID NOs: 1146, 1147, 1148, 1148, 1149 and 1150

28. The isolated DNA molecule of claim 25 wherein said CTEND sequence comprises a CTEND sequence selected from SEQ ID NOs:223-266; 438-461; 629-676; 835-882; and 1085-1135.

29. The isolated DNA molecule of claim 26 wherein said CTEND sequence comprises a CTEND sequence selected from SEQ ID NOs: 223-266; 438-461; 629-676; 835-882; and 1085-1135.

30. An isolated DNA molecule encoding a fusion peptide comprising a GBSS GLASS domain operably linked to a LINKR and a catalytic domain from a functional protein that synthesizes an α-1,4 glucan or an α-1,3 glucan, or an α-1,6 glucan, said fusion peptide being capable of modifying the glucan structure of a starch producing organism when starch is produced by said organism or part thereof in the presence of said fusion peptide.

31. The DNA molecule of claim 30 wherein said fusion peptide comprises a GLASS and/or a LINKR sequence of SEQ ID NOs:75-120; 284-335, 475-526; 682-732; 933-982 and/or 121-171, 336-386, 527-577, 733-783, and 983-1033.

32. An isolated DNA molecule encoding a polypeptide with glucan association properties of a maize GBSS enzyme capable of modification of starch metabolism in a plant or plant cell, said DNA comprising a molecule selected from the group consisting of:
(a) a DNA molecule encoding a protein domain having the amino acid SEQ ID No.1
(b) a DNA molecule comprising a corresponding nucleotide sequence from SEQ ID No.1141
(c) a DNA molecule comprising a nucleotide sequence differing from the sequence of the DNA molecules of (a) or (b) due to the degeneracy of the genetic code,
(d) a DNA molecule comprising a DNA sequence which hybridizes to any one of the DNA molecules of (a), (b) or (c) or fragment thereof, and which is equal to or more than 80% homologous or identical to the DNA molecule of (a), (b), or (c), or fragment thereof, wherein said DNA sequence encodes a polypeptide with Glucan Association Domain (Domain A) of a GBSS enzyme.

33. An isolated DNA molecule encoding a polypeptide with a glycosyl transferase function of a soluble or granule bound maize SS enzymes capable of modifying starch metabolism in a plant or plant cell, said DNA molecule being selected from the group consisting of:
(a) a DNA molecule encoding a protein domain comprising an amino acid of SEQ ID NOs:1, 2, 3, 4, and 149.
(b) a DNA molecule comprising the corresponding nucleotide sequence of SEQ ID NOs: 1141, 11142, 1143, 1144, and 1145,
(c) a DNA molecule comprising a nucleotide sequence differing from the sequence of the DNA molecules of (a) or (b) due to the degeneracy of the genetic code,
(d) a DNA molecule comprising a DNA sequence which hybridizes to any one of the DNA molecules of (a), (b) or (c), or fragment thereof, and which is equal to or more than 80% homologous or identical to the DNA molecule of (a), (b), or (c), or fragment thereof, wherein said DNA sequence encodes a polypeptide with a glycosyl transferase domain of a SS enzyme.

34. A recombinant DNA molecule comprising a DNA molecule of any of the above claims comprising a maize GBSS nucleotide coding region encoding for an amino acid sequence of SEQ. ID NOs. 101-146 fused with a corresponding coding region of a maize SS enzyme that encode for an amino acid sequence of SEQ. ID NOs: 35-74; 121-171; 172-222; 223-266; 268-283; 284-335; 336-386; 387-437; 438-461; 463-474; 475-526; 527-577; 578-628; 629-676; 678-681; 682-732; 733-834; 835-882; 884-932; 933-982; 1034-1084; and 1085-1135.

35. A recombinant DNA molecule comprising a DNA molecule of any of the above claims comprising a GLYTR, LINKR or CTEND domain DNA sequence selected from any one of SEQ ID NOs: 172-222; 387-437; 578-628; 784-834; 1034-1084, 121-171; 336-386; 527-577; 733-783; 983-1033 or 223-266; 438-461; 629-676; 835-882; 1085-1135 operably linked in any order with a corresponding DNA sequence that encodes for a glucan association domain from any one of SEQ ID NOs: 75-120; 284-335; 475-526; 682-732; 933-982.

36. A recombinant DNA molecule comprising a DNA molecule of any of the above claims comprising a DNA sequence differing from the sequence of any of the DNA molecules of SEQ ID NOs: 34-1150 due to the degeneracy of the genetic code, and/or protein or polypeptide originating from a different source, such as a plant species other than plant species such as maize, bacteria (e.g. E. Coli), Yeast, algae (Chlamydomonas), or fungus.

37. A recombinant DNA molecule comprising a DNA molecule of any of the above claims comprising a wherein the DNA sequence is selected from the group consisting of a coding region of a glucan association domain of SEQ ID NOs:75-120; 284-335; 475-526; 682-732; and 933-982 fused with a coding region of any glucan transferases listed in table XXXVII.

38. Method of expressing a starch synthase fusion proteins or polypeptides in a plant, in which the starch synthase protein or polypeptide domains are expressed as a fusion with a glucan association domain of granule bound starch synthase.

39 A method according to any one of the preceding claims, in which the protein or polypeptide is heterologous with respect to the plant in which the fusion is expressed.

40. Method according to any one of the preceding claims, comprising the steps of:
providing a genetic construct comprising at least one nucleotide sequence encoding the desired protein domain or polypeptide domain combined with at least one nucleotide sequence encoding a glucan association domain of GBSS, so that the construct encodes a fusion of the desired protein/polypeptide and at least one glucan association domain;
transforming a plant with said genetic construct;
expressing said genetic construct in the plant.

41. Method according to any one of the preceding claims in which the protein or polypeptide or recombinant protein or recombinant polypeptide is an enzyme.

42. Method according to claim 41, in which the enzyme is an enzyme that can interact and associate with starch or starch granules, or facilitate or be entrapped in starch or starch granules, and is capable of at least one of modifying, increasing, decreasing, altering or influencing starch structure or starch synthesis.

43. A vector comprising a DNA molecule according to any of the preceding claims.

44. A vector according to claim 43, wherein the DNA molecule is linked in sense orientation to DNA elements ensuring transcription of a translatable RNA in a prokaryotic or an eukaryotic cell.

45. A host cell comprising a vector according to claim 43.

46. A plant cell comprising a DNA molecule according to any one of the preceding claims linked to a heterologous promoter.

47. A plant comprising a plant cell according to claim 46.

48. The plant of claim 47, which is a cereal, such as maize, rice, wheat, barley, oats, or a root crop, such as potato, sweet potato, cassaya, yam, taro, or other starch producing plant, such as peas or banana.

49. A plant according to claim 47 wherein said plant contains or produces starch or starch granules in at least one of its parts, including its seeds, leaves, roots (tubers), tubers, stems, stalks, fruits, grains or flowers.

50. A plant according to claim 49 wherein said elements include a homologous or heterologous promoter specific for expression of said DNA molecule in said at least one of its parts.

51. A seed from the plant of claim 49, capable of expressing said recombinant molecule.

52. A modified starch derived from cells of a plant of any of the preceding claims.

53. Food or feed comprising a modified starch of claim 52.
